(19) United States
(12) Patent Application Publication
(10) Pub. No.: US 2024/0299536 A1
(43) Pub. Date: Sep. 12, 2024

(54) NANOPARTICLE COMPOSITIONS FOR SUSTAINED THERAPY

(71) Applicant: UTI Limited Partnership, Calgary (CA)

(72) Inventor: Pedro SANTAMARIA, Calgary (CA)

(21) Appl. No.: 18/645,030

(22) Filed: Apr. 24, 2024

Related U.S. Application Data
(62) Division of application No. 15/572,137, filed on Nov. 6, 2017, now Pat. No. 12,011,480.
(60) Provisional application No. 62/296,032, filed on Feb. 16, 2016.
(60) Provisional application No. 62/273,953, filed on Dec. 31, 2015.
(60) Provisional application No. 62/157,933, filed on May 6, 2015.

(51) Int. Cl. A61K 39/385; A61K 39/00; A61K 47/54; A61K 47/60; A61K 47/69

(52) U.S. Cl. CPC A61K 39/385; A61K 39/0008; A61K 47/545; A61K 47/60; A61K 47/6909; A61K 47/6929; A61K 47/6935; A61K 2039/57; A61K 2039/60; A61K 2039/627

(57) ABSTRACT

This disclosure provides compositions and methods for promoting the formation, expansion and recruitment of T_{R}1 cells and/or B cells in an antigen-specific manner and treating diseases and disorders in a subject in need thereof.

### CROSS-REFERENCE TO RELATED PATENT APPLICATIONS

This application is a divisional of U.S. application Ser. No. 15/572,137, filed Nov. 6, 2017, which is a national stage entry under 35 U.S.C. § 371 of International Application No. PCT/IB2016/000691, filed May 6, 2016, which claims priority under 35 U.S.C. § 119(e) to U.S. Provisional Application Nos. 62/157,933, 62/273,953, and 62/296,032, filed May 6, 2015, Dec. 31, 2015, and Feb. 16, 2016, respectively, the contents of each are incorporated herein by reference in their entireties.

### INCORPORATION OF SEQUENCE LISTING

The sequence listing that is contained in the file named “PTI0007-515D1-US,” which was created Apr. 24, 2024, and which has a file size of 464 kilobytes as measured in Microsoft Windows operating system, is filed electronically herewith and incorporated herein by reference.

### BACKGROUND

Throughout and within this disclosure are technical and patent publications, referenced by an identifying citation or by an Arabic number. The full bibliographic citation corresponding to the Arabic number is found in the specification, preceding the claims. The disclosures of all references cited herein are incorporated by reference into the present application to more fully describe the state of the art to which this disclosure pertains.

A wide variety of diseases implicate improper immune function in pathogenesis or exacerbation of symptoms. While a wide variety of immunotherapies exist, they are often coupled with off target effects due to lack of targeting specificity and/or adverse side effects.

Thus a need exists with respect to finding safe and effective therapies for these disorders. This disclosure satisfies this need and provides related advantages as well.

### SUMMARY OF THE DISCLOSURE

This disclosure relates to a nanomedicine, which in one aspect, is a complex comprising a nanoparticle core coupled to a plurality of disease-relevant antigen-MHC complexes (abbreviated herein as “pMHCs” or “pMHC complexes”), that are useful for expanding and differentiating T cell populations and treating disease when administered in an effective amount to a subject. The nanoparticle core comprises a variety of compositions or components, as describe in more detail herein. In some aspects, the nanoparticle core has a diameter selected from the group of from about 1 nm to about 100 nm; from about 1 nm to about 75 nm; from about 1 nm to about 50 nm; from about 1 nm to about 25 nm; from about 1 nm to about 25 nm; from about 5 nm to about 100 nm; from about 5 nm to about 50 nm; or from about 5 nm to about 25 nm, or from about 15 nm to about 25 nm, or about 20 nm. In some embodiments, the nanoparticles core has a diameter of from about 25 nm to about 60 nm, or from about 25 nm to about 50 nm, or from about 20 nm to about 40 nm, or from about 15 nm to about 50 nn, or from about 15 nm to about 40 nm, or from about 15 nm to about 35 nm, or from about 15 nm to about 30 nm, or from about 15 nm to about 25 nm, or alternatively about 15 nm, or about 20 nm, or about 25 nm, or about 30 nm, or about 35 nm, or about 40 nm.

In some aspects, the number of pMHCs per nanoparticle core (referred to herein as the “valency” of the nanoparticle complex) may range between about 1 pMHC complex to 1 nanoparticle core to about 6000 pMHC complexes to 1 nanoparticle core, or alternatively between about 10:1 to about 6000:1, or alternatively between about 11:1 to about 6000:1, or alternatively between about 12:1 to about 6000:1, or alternatively at least 2:1, or alternatively at least 8:1, or alternatively at least 9:1, or alternatively at least 10:1, or alternatively at least 11:1, or alternatively at least 12:1. In some aspects, the number of pMHCs per nanoparticle core is from about 10:1 to about 6000:1, or from about 20:1 to about 5500:1, or alternatively from about 10:1 to about 5000:1, or alternatively from about 10:1 to about 4000:1, or alternatively from about 10:1 to about 3500:1, or alternatively from about 10:1 to about 3000:1, or alternatively from about 10:1 to about 2500:1, or alternatively from about 10:1 to about 2000:1, or alternatively from about 10:1 to about 1500:1, or alternatively from about 10:1 to 1000:1, or alternatively from about 10:1 to about 500:1, or alternatively from about 10:1 to about 100:1, or alternatively from about 20:1 to about 50:1, or alternatively from about 25:1 to about 60:1; alternatively from about 30:1 to about 50:1, or alternatively from about 35:1 to about 45:1, or alternatively about 40:1.

In some aspects, the nanoparticle core has a defined valency per surface area of the core, also referred to herein as “density.” In these aspects, the pMHC density per nanoparticle is from about 0.025 pMHC/100 nm² to about 100 pMHC/100 nm² of the surface area of the nanoparticle core, or alternatively from about 0.406 pMHC/100 nm² to about 50 pMHC/100 nm²; or alternatively from about 0.05 pMHC/100 nm² to about 25 pMHC/100 nm². In certain aspects, the pMHC density per nanoparticle is from about 0.4 pMHC/100 nm² to about 25 pMHC/100 nm², or from about 0.4 pMHC/100 nm² to about 20 pMHC/100 nm², or from about 0.4 pMHC/100 nm² to about 15 pMHC/100 nm², or from about 0.4 pMHC/100 nm² to about 14 pMHC/100 nm², or from about 0.4 pMHC/100 nm² to about 13 pMHC/100 nm², or from about 0.4 pMHC/100 nm² to about 12 pMHC/100 nm², or from about 0.4 pMHC/100 nm² to about 11.6 pMHC/100 nm², or from about 0.4 pMHC/100 nm² to about 11.5 pMHC/100 nm², or from about 0.4 pMHC/100 nm² to about 11 pMHC/100 nm², or from about 0.4 pMHC/100 nm² to about 10 pMHC/100 nm², or from about 0.4 pMHC/100 nm² to about 9 pMHC/100 nm², or from about 0.4 pMHC/100 nm² to about 8 pMHC/100 nm², or from about 0.4 pMHC/100 nm² to about 7 pMHC/100 nm², or from about 0.4 pMHC/100 nm² to about 6 pMHC/100 nm², or from about 0.4 pMHC/100 nm² to about 5 pMHC/100 nm², or from about 0.4 pMHC/100 nm² to about 4 pMHC/100 nm², or from about 0.4 pMHC/100 nm² to about 3 pMHC/100 nm², or from about 0.4 pMHC/100 nm² to about 2.5 pMHC/100 nm², or from about 0.4 pMHC/100 nm² to about 2 pMHC/100 nm², or from about 0.4 pMHC/100 nm² to about 1.5 pMHC/100 nm².

In another aspect, the nanoparticle may have a pMHC density of from about 0.22 pMHC/100 nm² to about 10 pMHC/100 nm², or from about 0.22 pMHC/100 nm² to about 9 pMHC/100 nm², or from about 0.22 pMHC/100 nm² to about 8 pMHC/100 nm², or from about 0.22 pMHC/100 nm² to about 7 pMHC/100 nm², or from about 0.22 pMHC/100 nm² to about 6 pMHC/100 nm², or from about 0.22 pMHC/100 nm² to about 5 pMHC/100 nm², or from about 0.22 pMHC/100 nm² to about 4 pMHC/100 nm², or from about 0.22 pMHC/100 nm² to about 3 pMHC/100 nm², or from about 0.22 pMHC/100 nm² to about 2 pMHC/100 nm², or from about 0.22 pMHC/100 nm² to about 1.5 pMHC/100 nm². In some aspects, the nanoparticle has a pMHC density of from about 0.22 pMHC/100 nm² to about 10 pMHC/100 nm², or 0.24 pMHC/100 nm² to about 9 pMHC/100 nm², or from about 0.26 pMHC/100 nm² to about 8 pMHC/100 nm², or from about 0.28 pMHC/100 nm² to about 7 pMHC/100 nm², or from about 0.24 pMHC/100 nm² to about 4 pMHC/100 nm², or from about 0.5 pMHC/100 nm² to about 3 pMHC/100 nm², or from about 0.6 pMHC/100 nm² to about 1.5 pMHC/100 nm². In a further aspect, the nanoparticle has a pMHC density of from about 0.4 pMHC/100 nm² to about 1.3 pMHC/100 nm², or alternatively from about 0.5 pMHC/100 nm² to about 0.9 pMHC/100 nm², or alternatively from about 0.6 pMHC/100 nm² to about 0.8 pMHC/100 nm².

In some embodiments, the nanoparticle can have a pMHC density of from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 pMHC/100 nm². In specific embodiments, the nanoparticle can have a pMHC density of from about 0.4 pMHC/100 nm² to about 1.5 pMHC/100 nm² or from about 0.4 pMHC/100 nm² to about 6 pMHC/100 nm² or from about 0.4 pMHC/100 nm² to about 12 pMHC/100 nm².

In yet another aspect, the nanoparticle has a pMHC density as defined herein of from about 0.4 pMHC/100 nm² to about 1.3 pMHC/100 nm², or alternatively from about 0.5 pMHC/100 nm² to about 0.9 pMHC/100 nm², or alternatively from about 0.6 pMHC/100 nm² to about 0.8 pMHC/100 nm², and further wherein the nanoparticle core has a diameter from about from about 25 nm to about 60 nm, or from about 25 nm to about 50 nm, or from about 20 nm to about 40 nm, or from about 15 nm to about 50 nn, or from about 15 nm to about 40 nm, or from about 15 nm to about 35 nm, or from about 15 nm to about 30 nm, or from about 15 nm to about 25 nm, or alternatively about 15 nm, or about 20 nm, or about 25 nm, or about 30 nm, or about 35 nm, or about 40 nm.

In some aspects, the nanoparticle core further comprises a plurality of co-stimulatory molecules, co-stimulatory antibodies, inhibitory receptor-blocking antibodies, and/or a plurality of cytokines coupled to the nanoparticle core.

Thus, certain aspects of the disclosure relate to a complex comprising, or alternatively consisting essentially of, or yet further consisting of, nanoparticle cores coupled to a plurality of pMHC complexes, wherein the nanoparticles cores optionally further comprise, or further consist thereof, or alternatively further consist essentially of one or more co-stimulatory molecules and/or one or more cytokines coupled to the nanoparticle core. For these compositions containing a plurality of the complexes, the pMHC complexes on each nanoparticle core are the same or different from each other; and/or the MHC of the pMHC complexes on each nanoparticle core are the same or different from each other; and/or the cytokines on each nanoparticle core are the same or different from each other; and/or the costimulatory molecules on each nanoparticle core are the same or different from each other; and/or the diameters of the nanoparticle cores are the same or different from each other; and/or the valency of the pMHC complexes on each nanoparticle core are the same or different from each other; and/or the density of the pMHC complexes on each nanoparticle core are the same or different from each other; and/or the valency of the co-stimulatory molecules on each nanoparticle core are the same or different from each other; and/or the valency of the cytokines on each nanoparticle core are the same or different from each other.

In certain aspects, provided herein are compositions comprising a plurality of the complexes provided herein. In some embodiments, the compositions further comprise a carrier, optionally a pharmaceutical carrier. In some embodiments, the compositions provided herein may optionally comprise one or more nanoparticle cores coupled to one or more co-stimulatory molecules and/or cytokines. Accordingly, in some embodiments, the compositions comprise, or alternatively consist essentially of, or yet further consist of: 1) a plurality of nanoparticle cores coupled to a plurality of antigen-MHC complexes wherein at least one portion of the nanoparticle cores further comprises one or more co-stimulatory molecules and/or one or more cytokines and a second portion of the nanoparticle cores do not further comprise a co-stimulatory molecule and/or a cytokine, and 2) a plurality of nanoparticle cores coupled to one or more co-stimulatory molecules and/or cytokines.

Further aspects of the disclosure relate to specific disease-relevant antigens, MHCs, and combinations thereof optimized for the treatment or prevention of disease in human patients and animals.

This disclosure also provides compositions and methods of use for any of the above complexes or compositions, each of which is optionally combined with a carrier, for example a pharmaceutically acceptable carrier.

This disclosure also provides methods for differentiating or triggering T-regulatory type 1 (T_{R}1) cell formation in a pMHC dose independent manner. Applicant has discovered that the pMHC density on the nanoparticle core regulates the ability of pMHC on the nanoparticle core to trigger T_{R}1 cell formation in a dose-independent manner, while pMHC dose regulates the magnitude of T_{R}1 cell expansion in a pMHC density-independent manner. Applicant has observed that the pMHC density threshold and the independent effects of pMHC density versus dose on T_{R}1 cell formation versus expansion are unexpected findings that could not have been anticipated based on conventional immunological knowledge in the art. These methods require contacting (in vitro or in vivo) the cognate T cells with an effective amount of a pMHC-NPor a composition disclosed herein. In certain aspects, the density-dependent methods relate to an activated T cell or a memory T cell being differentiated into a IL-10 producing cognate T_{R}1 cell optionally having the marker CD49b and/or Lag3 and/or a B cell being differentiated into a regulatory B cell by contacting the activated T cell or the memory T cell with an effective amount of the complex or composition disclosed herein. In some embodiments, the differentiated T_{R}1 cell binds to a B cell, thereby differentiating the B cell into a regulatory B cell. In certain aspects of the methods, the contacting is performed in vitro or in vivo. In some embodiments, the pMHC-NP or composition containing a plurality of the pMHC-NPs have pMHC-NPs having an average nanoparticle core diameter of from about 25 nm to about 60 nm, or from about 25 nm to about 50 nm, or from about 20 nm to about 40 nm, or from about 15 nm to about 50 nn, or from about 15 nm to about 40 nm, or from about 15 nm to about 35 nm, or from about 15 nm to about 30 nm, or from about 15 nm to about 25 nm, or alternatively about 15 nm, or about 20 nm, or about 25 nm, or about 30 nm, or about 35 nm, or about 40 nm. In some aspects, the nanoparticle core further comprises an outer coating or layer, wherein the diameter of the core and outer layer have an average diameter of from about 30 nm to about 75 nm, or from about 30 nm to about 70 nm, or from about 30 nm to about 60 nm, or from about 30 nm to about 50 nm, or about 40 nm. In some aspects, the nanoparticle has an average pMHC density of from about 0.4 pMHC/100 nm² to about 12 pMHC/100 nm², or from about 0.4 pMHC/100 nm² to about 11.6 pMHC/100 nm², or from about 0.4 pMHC/100 nm² to about 11.5 pMHC/100 nm², or from about 0.4 pMHC/100 nm² to about 11 pMHC/100 nm², or from about 0.4 pMHC/100 nm² to about 10 pMHC/100 nm², or from about 0.4 pMHC/100 nm² to about 9 pMHC/100 nm², or from about 0.4 pMHC/100 nm² to about 8 pMHC/100 nm², or from about 0.4 pMHC/100 nm² to about 7 pMHC/100 nm², or from about 0.4 pMHC/100 nm² to about 6 pMHC/100 nm², or from about 0.4 pMHC/100 nm² to about 5 pMHC/100 nm², or from about 0.4 pMHC/100 nm² to about 4 pMHC/100 nm², or from about 0.4 pMHC/100 nm² to about 3 pMHC/100 nm², or from about 0.4 pMHC/100 nm² to about 2.5 pMHC/100 nm², or from about 0.4 pMHC/100 nm² to about 2 pMHC/100 nm², or from about 0.4 pMHC/100 nm² to about 1.5 pMHC/100 nm².

Further aspects of the disclosure relate to methods to treat or prevent the relevant disease or conditions as disclosed herein by administering an effective amount of a pMHC-NP as disclosed herein. Also disclosed are methods of detecting the presence and efficacy of treatment with the pMHC-NP complexes and compositions as disclosed herein.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1B show schematics of NP-complexes. FIG. 1A is a schematic of a single-chain pMHC-class I expression construct (top) and a representative flow cytometric profile of the binding of the corresponding pMHC tetramer (fluorochrome-labeled) to cognate CD8+ T-cells. FIG. 1B is a schematic showing the linkers and two dimensional structure of NP-complexes. As can be seen, one NP can contain the same antigen complexed to the nanoparticle core through various chemical linkers.

FIG. 2 shows the structure of a typical pMHC class II monomer (top) and a representative FACS profile of cognate CD4+ T-cells stained with the corresponding pMHC tetramer or left unstained.

FIG. 3 shows the chemical structure of Dendri-Graft Poly-L-Lysines Generation 3 (DGLs G3).

FIG. 4 shows the synthesis of G3 Dendri-Graft Poly-L-Lysines functionalized with PEG-Azido (DGLN).

FIG. 5 shows the synthesis of pMHC-DGLN.

FIG. 6 shows native and denaturing PAGE analysis of pMHC-DGLN conjugates.

FIG. 7 shows AFM analysis of V7CHO-DGLN.

FIGS. 8A-8B show that V7CHO-DGLN have powerful agonistic properties on cognate CD8+ T-cells.

FIGS. 9A-9N show pMHC-NPs relevant for T1D or EAE expand cognate disease-suppressing T_{R}1-like CD4⁺ T cells in vivo. FIGS. 9A and 9B show tetramerstaining profiles (FIG. 9A) and percentages of tetramer⁺CD4⁺ T cells (FIG. 9B). Data correspond to pre-diabetic NOD females treated for 5 weeks (blood: n=5, 8 and 6; spleen: n=5, 18 and 6, respectively). Tet, tetramer. FIG. 9C shows tetramer-staining of splenic CD4⁺ T cells from treated or untreated NOD Foxp3-eGFP mice. FIG. 9D shows the tetramer⁺CD4⁺ T cells of 2.5mi/IA^{g7}-NP-treated mice display a T_{R}1-like phenotype. FIG. 9E shows incidence of diabetes in T-cell-reconstituted NOD scid hosts transfused with CD4⁺ T cells from different donors±2.5mi/IA^{g7}-NPs (n=11, 5, 7 and 6 from top). FIG. 9F shows percentages of tetramer⁺CD4⁺ T cells in 2.5mi/IA^{g7}-NP-treated or untreated NOD scid hosts (n=4-5 per group). FIG. 9G shows incidence of disease reversal in diabetic mice treated with pMHC-NPs (n=9, 7, 7, 7 from top left to right), or IGRP₄₋₂₂ peptide (Burton, B. R. et al. (2014) Nature Commun. 5:4741-4747) and IGRP₄₋₂₂ peptide-NP (n=9). FIG. 9H shows percentage of tetramer⁺CD4⁺ T cells in diabetic mice at onset, in response to 2.5mi/IA^{g7}-NP therapy and age-matched non-diabetic controls (n=8, 6, 2 and 7 from left). FIG. 9I shows insulitis scores (n=6, 4, 3 and 6 from left). Bottom, representative images. FIGS. 9J-9M show C57BL/6 mice were immunized with pMOG₃₅₋₅₅. FIG. 9J shows EAE scores of mice treated from day 14 (n=4 each). FIG. 9K shows EAE scores of mice treated from day 21 (n=10, 7 and 3 from top). FIG. 9L shows percentage of tetramer⁺CD4⁺ T cells in spleen and blood of mice from FIGS. 9J and 9K (n=13, 14 and 5 from top). FIG. 9M shows representative flow profiles of CD4⁺ T cells from mice in FIGS. 9J and 9K. FIG. 9N shows representative microglial IBA1 stainings and relative rank scores in the cerebellum of mice from FIG. 9K (n=4-5). P values were calculated via Mann-Whitney U-test, log-rank (Mantel-Cox) test or two-way ANOVA. Error bars, s.e.m.

FIGS. 10A-10H show therapeutic effects are disease-specific and dependent on both pMHC and nanoparticles. FIGS. 10A-10F show C57BL/10.M HLA-DR4-IE transgenic mice immunized with bovine collagen. FIG. 10A, left, shows changes in joint swelling (top) and clinical scores (bottom) in response to uncoated NPs, pMHC-NPs, peptide s.c. (Burton, B. R. et al. (2014) Nature Commun. 5:4741-4747) or peptide-coated MPs i.v (Getts, D. R. et al. (2012) Nature Biotechnol. 30:1217-1224). Treatment was initiated when joint swelling reached 130% of baseline (data normalized to the initiation of treatment (100% value)) (n=4, 4, 4 and 8 from top). Right, percentage increase in joint swelling relative to pre-immunization baseline (100% value). FIG. 10B shows representative haematoxylin and eosin (first row) and O-safranin/fast-green/haematoxylin (second and third rows) knee joint staining images. Third row shows enlarged images of lacunae on the bone and meniscal articular surfaces. 1, panus formation; 2, cellular infiltration of the meniscus; 3, bone erosion; 4, proteoglycan depletion; 5, loss of chondrocyte/lacunnae. FIG. 10C shows average pathology scores (n=3-4 per group). FIG. 10D shows percentage of tetramer⁺CD4⁺ T cells. FIG. 10E shows representative flow cytometry profiles for T_{R}1 markers in mCII₂₅₉₋₂₇₃/DR4-NP-treated. FIGS. 10F-10H show C57BL/6 IAb^{null} HLA-DR4-IE-transgenic mice immunized with hPLP. FIG. 10F shows changes in EAE scores ((n=5, 4, 13 (4-9 per group), 5, 19 (4-5 per group, see also FIG. 9H), 4 and 5 from top). FIG. 10G shows percentage of tetramer⁺CD4⁺ T cells in the spleen of mice from FIG. 10F (n=4, 5, 4, 6, 15, 3 and 3 from left). FIG. 10H shows representative flow cytometry profiles for T_{R}1 markers. Data were compared using Mann-Whitney U-test or two-way ANOVA. Error bars, s.e.m.

FIGS. 11A-11H show disease reversal involves effects of T_{R}1 cytokines on cognate B cells and local CD11b+ cells, without compromising systemic immunity. FIG. 11A shows blood glucose levels in diabetic NOD mice treated with 2.5mi/IA^{g7}-NPs and blocking antibodies (n=8, 4, 6, 6, 5 and 4 from top to right). FIG. 11B shows expression of IL-10 (eGFP) and upregulation of CD5 and CD1d by eGFP⁻ 2.5mi-pulsed splenic B cells from NOD Il10^{GFP} donors in 2.5mi/IA^{g7}-NP-treated NOD hosts. FIG. 11C shows averaged results from FIG. 11B (n=4, 3, 3 and 7 from left). FIG. 11D shows incidence of diabetes in T-cell-reconstituted NOD scid hosts left alone or transfused with PLN CD19⁺ cells (n=7, 13 and 7 from top). FIG. 11E shows incidence of diabetes in T-cell-reconstituted NOD scid hosts transfused with CD19+ and/or CD4+ cells (n=7, 6, 3, 7, 8, 11 and 13 from top). FIG. 11F shows cytokine and chemokine profiles of PLN and MLN CD11b⁺ cells from 2.5mi/IA^{g7}-NP-treated NOD mice in response to LPS (n=3-4 each). FIG. 11G shows percentage of tetramer+CD4+ T cells in the spleens (left), and viral titres in the ovaries (right) of treated compared with untreated NOD mice 4 and 14 days after vaccinia virus infection (n=3 per group). FIG. 11H shows percentages of tetramer⁺CD4⁺ T cells in the spleens (left) and serum anti-dinitrophenyl (DNP) antibody titres (right) in treated and untreated NOD mice immunized with keyhole limpet haemocyanin (KLH)-DNP (n=3-5 per group). Data were compared using Mann-Whitney U-test, log-rank test or two-way ANOVA. Error bars, s.e.m.

FIGS. 12A-12G show the T_{R}1-like CD4⁺ T cells arising in response to pMHCII-NPs are derived from antigen-experienced precursors. FIG. 12A, Percentage of tetramer⁺CD4⁺ T cells in hyperglycaemic NOD G6pc2^{-/-} compared with NOD mice treated with IGRP₄₋₂₂/IA^{g7}-(n=4 and 7) or 2.5mi/IA^{g7}-NPs (n=6 and 9). FIG. 12B shows blood glucose levels in hyperglycaemic NOD G6pc2^{-/-} mice in response to pMHC-NP therapy (n=4-6 per group). FIG. 12C shows upregulation of T_{R}1 transcripts by anti-CD3/anti-CD28 mAb-activated eGFP-CD4+ T cells from BDC2.5 NOD Foxp3-eGFP mice in response to different in vitro stimuli (n=4 mice each). FIG. 12D shows changes in T_{R}1-relevant transcripts in naive or memory BDC2.5 CD4⁺ T cells in response to 2.5mi/IA^{g7}-NPs in vivo (n=6, 6, 5 and 4 from left). FIG. 12E shows LAG-3 and CD49b profiles (blue; compared with isotype control in red) of Thy^{1b+} cells from FIG. 12D. FIG. 12F shows proliferation of CFSE-labelled memory BDC2.5 CD4+ T cells in NOD.Thy1^{a} hosts in response to 2.5mi/IAg7-NPs. FIG. 12G shows incidence of diabetes in T-cell-reconstituted NOD scid hosts transfused with naive or memory BDC2.5 CD4⁺ T cells and treated with bi-weekly doses of 2.5mi/IA^{g7}-NPs (n=4 and 3) or left untreated (n=4 and 6). P values were calculated via Mann-Whitney U-test or log-rank (Mantel-Cox) tests. Error bars, s.e.m.

FIGS. 13A-13I show human T1D-relevant pMHC-NPs expand cognate T_{R}1-like CD4⁺ T cells in human PBMC-engrafted NSG hosts. FIG. 13A shows expansion of cognate CD4⁺ T cells by GAD_{555-567(557I)}/DR4-NPs (top) or PPI_{76-90(88S)}/DR4-NPs (bottom) in NSG mice engrafted with PBMCs from DR4⁺ T1D patients. FIG. 13B shows CD49b and LAG-3 marker expression on the sample at the bottom of FIG. 13A. FIG. 13C shows expansion of cognate T_{R}1-like CD4⁺ T cells in NSG mice engrafted with PBMCs from DR3+ T1D patients in response to IGRP₁₃₋₂₅/DR3-NP-therapy. FIG. 13D shows percentages (left) and numbers (right) of tetramer⁺CD4⁺ T cells in mice engrafted with T1D PBMCs in response to treatment (n for spleen and PLN per treatment=9/6, 7/6 and 14/1 from left legend). FIG. 13E shows expression of Il10 mRNA in IGRP₁₃₋₂₅/DR3 tetramer⁺CD4⁺ T cells from mice treated with IGRP₁₃₋₂₅/DR3-NPs (n=3 each). FIG. 13F shows the PLNs of responder mice contained increased numbers of lymphocytes compared to the other groups (n=6, 3, 4, 3 from top legend). FIGS. 13G and 13H show correlation between the absolute numbers of IGRP₁₃₋₂₅/DR3 tetramer⁺ cells in the PLNs (FIG. 13G) or spleen (FIG. 13H) and the percentage or absolute number of PLN or splenic B cells in IGRP₁₃₋₂₅/DR3-NP-treated mice (n=6 and 7). FIG. 13I shows secretion of IL-10 by LPS-stimulated CD19⁺ cells (ex vivo, for 24 h) isolated from the PLNs or spleens of hPBMC-engrafted NSG mice treated with IGRP₁₃₋₂₅/DR3-NPs (n=3 each). P values were calculated by Mann-Whitney U-test or Pearson correlation test. Error bars, s.e.m.

FIGS. 14A-14N show sustained expansion of cognate T_{R}1-like CD4⁺ T cells by pMHCII-NP therapy restores normal glucose homeostasis in diabetic NOD mice by suppressing antigen presentation and the activation of non-cognate autoreactive T cells in the PLNs and the progression of insulitis. FIG. 14A, top left, shows expansion of cognate CD4⁺ T cells by 2.5mi/IA^{g7}-NPs in anti-CD25 mAb-treated NOD Foxp3-eGFP mice. Data correspond to 8-week-old mice treated three times a week with 500 μg of a depleting anti-CD25 mAb i.p. or control anti-HPRN mAbs, followed by 10 doses of 2.5mi/IA^{g7}-NPs starting at 10 weeks of age (two doses per week; n=4 mice each). Bottom, the tetramer⁺CD4⁺ T cells from anti-CD25 mAb-treated mice express T_{R}1 markers. Right, percentage of circulating FOXP3⁺eGFP⁺CD4⁺ (top) and CD25⁺CD4⁺ cells (bottom). FIG. 14B shows tetramer⁺CD4⁺ T cells sorted from 2.5mi/IA^{g7}-NP-treated mice proliferate and produce IL-10 and, to a lesser extent IFNγ in response to stimulation with 2.5mi peptide-pulsed DCs (n=3 mice). FIG. 14C shows representative cell surface CD49b and LAG-3 profiles on tetramer⁺CD4⁺ T cells from BDC2.5 NOD Foxp3-eGFP mice compared with tetramer-CD4⁺ T cells from transgenic or wild-type NOD mice (n=4). FIG. 14D shows upregulation of CD49b and LAG-3 on anti-CD3/anti-CD28 mAb-activated BDC2.5 CD4⁺ T cells from BDC2.5 NOD Foxp3-eGFP mice in response to 2.5mi/IA^{g7}-NP (25 μg pMHC per ml) versus 2.5mi peptide (10 μg ml⁻¹) or 2.5mi/IA^{g7} monomers (25 μg pMHC per ml). FIG. 14E shows upregulation of eGFP (IL-10) in anti-CD3/anti-CD28 mAb-activated BDC2.5 CD4⁺ T cells from BDC2.5 NOD Il10^{GFP} mice in response to 2.5mi/IA^{g7}-NP as a function of CD49b and LAG-3 expression. FIG. 14F shows expression of eGFP (IL-10) in the CD4⁺ T cells of 2.5mi/IA^{g7}-NP-treated NOD Il10^{GFP} mice (2 doses per week for 5 weeks) as a function of CD49b and LAG-3 expression (left, representative profiles; right, eGFP MFI values) (n=8). FIG. 14G shows proliferation of CFSE-labelled 8.3-TCR-transgenic CD8⁺ T cells (IGRP₂₀₆₋₂₁₄/NRP-V7-specific) in response to 2.5 mi/NRP-V7-peptide-pulsed or unpulsed DCs in the presence of tetramer− or tetramer⁺ CD4⁺ T cells from 2.5mi/IA^{g7}-NP-treated mice and in the presence or absence of cytokineblocking mAbs, rat IgG (negative control) or 1-methyl-l-tryptophan (1-MT; an IDO inhibitor). Data correspond to average of proliferated cells in 3-7 experiments per condition. FIG. 14H shows changes in blood glucose levels of spontaneously hyperglycaemic (>11 mM) female NOD mice treated with 2.5mi/IA^{g7}-NP, IGRP₄₋₂₂/IA^{g7}-NP, IGRP₁₂₈₋₁₄₅/IA^{g7}-NP or HEL₁₄₋₂₂/IA^{g7}-NP (n=6-9 per group), IGRP₄₋₂₂ peptide or IGRP₄₋₂₂ peptide-NPs (n=9, 4-5 each). Mice received two doses per week until irreversibly hyperglycaemic or normoglycaemic for 4 consecutive weeks, at which point treatment was withdrawn. FIG. 14I shows incidence and timing of disease relapse in hyperglycaemic female NOD mice rendered stably normoglycaemic by treatment with 2.5mi/IA^{g7}-NP, IGRP₄₋₂₂/IA^{g7}-NP or IGRP₁₂₈₋₁₄₅/IA^{g7}-NPs upon treatment withdrawal (after 4 consecutive weeks of normoglycaemia). Data correspond to responder mice in FIG. 9G. FIG. 14J shows post-prandial serum insulin levels in pMHC-NP-treated mice that reverted to normoglycaemia until 50 weeks of age (n=6) versus newly diabetic (n=12) and non-diabetic age-matched untreated controls (n=10). FIG. 14K shows intra-peritoneal glucose tolerance tests (IPGTT) of the mice in FIG. 14H. FIG. 14L shows areas under the curve (AUC) in the IPGTTs shown in FIG. 14K. FIG. 14M shows IPGTT serum insulin levels corresponding to the mice in FIG. 14K. FIG. 14N shows proliferation of CFSE-labelled IGRP₂₀₆₋₂₁₄-reactive 8.3-CD8+ T cells in the PLNs compared with MLNs of 2.5mi/IA^{g7}-NP-treated mice that reverted to normoglycaemia until 50 weeks of age, non-diabetic age-matched untreated controls and newly diabetic mice. Left panels show representative FACS profiles. Right panel compares percentages of proliferated cells in the PLNs after subtraction of the background proliferation values in non-draining MLNs (n=6-8 mice per group). P values were calculated by Mann-Whitney U-test, log-rank (Mantel-Cox) test or two-way ANOVA. Data are averages±s.e.m.

FIGS. 15A-15H show nanoparticles coated with different T1D-relevant pMHCII complexes expand cognate T_{R}1-like CD4⁺ T cells in vivo to similar extent, regardless of epitope dominance or role of the target T-cell specificity in the disease process. FIG. 15A shows percentage of tetramer⁺CD4⁺ T cells in the PLN, MLN and bone marrow (BM) of 2.5mi/IA^{g7}-NP-treated mice that reverted to normoglycaemia until 50 weeks of age (n=5-6 mice per lymphoid organ) or relapsed (n=1-2) compared with newly diabetic (n=5-6) and non-diabetic age-matched untreated controls (n=4-6). FIG. 15B shows percentage of tetramer⁺CD4⁺ T cells in the splenic CD4⁺ T cells of 2.5mi/IA^{g7}-NP-treated mice that reverted to normoglycaemia until 50 weeks of age or of age-matched non-diabetic untreated mice, stained with two TID-relevant but non-cognate pMHCII tetramers (n=3-4 per group). FIG. 15C shows percentage of tetramer⁺CD4⁺ T cells in blood, spleen, PLN, MLN and bone marrow of IGRP₄₋₂₂/IA^{g7}-NP-treated mice that reverted to normoglycaemia until 50 weeks of age (n=5-6 mice per lymphoid organ) compared with newly diabetic (n=5-8) and non-diabetic age-matched untreated controls (n=4-6). FIG. 15D shows percentage of tetramer⁺CD4⁺ T cells in blood, spleen, PLN, MLN and bone marrow of IGRP₁₂₈₋₁₄₅/IA^{g7}-NP-treated mice that reverted to normoglycaemia until 50 weeks of age (n=5-7 mice per lymphoid organ) compared with newly diabetic (n=4-7) and non-diabetic age-matched untreated controls (n=5-7). FIG. 15E shows representative IGRP₄₋₂₂/IA^{g7}, IGRP₁₂₈₋₁₄₅/IA^{g7} and GPI₂₈₂₋₂₉₂/IA^{g7} tetramer staining profiles for splenic CD4⁺ T cells from IGRP₄₋₂₂/IA^{g7}-NP- and IGRP₁₂₈₋₁₄₅/IA^{g7}-NP-treated compared with untreated NOD mice. FIG. 15F shows percentages of blood CD4⁺ T cells of IGRP₄₋₂₂/IA^{g7}-NP- or IGR_{P128-145}/IA^{g7}-NP-cured, HEL₁₄₋₂₂/IA^{g7}-NP-treated and age-matched non-diabetic untreated mice stained with non-cognate pMHCII tetramers (n=3-7 per group). FIG. 15G shows the tetramer⁺CD4⁺ T cells of mice treated with IGRP₁₂₈₋₁₄₅/IA^{g7}-NP (top) and IGRP₄₋₂₂/IA^{g7}-NP (bottom) proliferate and produce IL-10 specifically in response to stimulation with IGRP₄₋₂₂ or IGRP₁₂₈₋₁₄₅-peptide-pulsed DCs, respectively (n=3 mice each), cpm, counts per minute. FIG. 15H shows percentages of IGRP₄₋₂₂/IA^{g7} tetramer⁺CD4⁺ T cells in blood, spleen, PLN, MLN and bone marrow of NOD mice at the onset of hyperglycaemia or upon treatment with IGRP₄₋₂₂/IA^{g7}-NPs, or IGRP₄₋₂₂ peptide or IGRP₄₋₂₂ peptide-coated nanoparticles (n=5-9 mice per organ). P values were calculated by Mann-Whitney U-test. Data are averages±s.e.m.

FIGS. 16A-16F show EAE-relevant pMHCII-NPs expand cognate IL-10-secreting T_{R}1-like CD4⁺ T cells and ameliorate established clinical and pathological signs of EAE. FIGS. 16A and 16B show changes in the average weights of C57BL/6 mice immunized with pMOG₃₅₋₅₅ and treated with pMOG₃₈₋₄₉/IA^{b}-NPs or uncoated nanoparticles starting on days 14 (FIG. 16A) or 21 (FIG. 16B) after immunization. FIG. 16C shows percentage of pMOG₃₈₋₄₉/IAb tetramer⁺CD4⁺ T cells in peripheral lymph nodes, bone marrow and central nervous system (CNS) of mice from FIGS. 16A and 16B. FIG. 16D shows the tetramer⁺CD4⁺ T cells of pMOG₃₈₋₄₉/IA^{b}-NP-treated mice proliferate and produce IL-10 and, to a lesser extent, IFNγ in response to stimulation with pMOG₃₈₋₄₉ peptide-pulsed DCs. FIG. 16E, left and middle, shows representative luxol fast blue (LFB)/H&E cerebellum staining images from untreated and treated mice from FIG. 16B showing presence of inflammatory foci and areas of demyelination (red arrows). Right, average number of inflammatory foci per section. Data corresponds to 4 untreated and 5 treated mice. FIG. 16F shows representative LFB/H&E-stained spinal cord sections from mice in FIG. 16B. Data were compared with Mann-Whitney U-test. Data are averages±s.e.m.

FIGS. 17A-17I show EAE- or CIA-relevant pMHCII-NPs expand cognate T_{R}1-like CD4⁺ T cells and ameliorate clinical and pathological signs of EAE or CIA in HLA-DR4-IE-transgenic C57BL/6 IAb^{null} or C57BL/10.M mice. FIG. 17A shows changes in the average EAE scores of HLA-DR4-IE-transgenic C57BL/6 IAb^{null} mice immunized with hPLP₁₇₅₋₁₉₂ or hMOG₉₇₋₁₀₈ and treated with hPLP₁₇₅₋₁₉₂/DR4-IE or hMOG₉₇₋₁₀₈/DR4-IE-NPs or uncoated nanoparticles starting on the day when mice reached a score of 1.5 (to synchronize the groups for disease activity) (n=3-4 per group). FIG. 17B shows percentage of tetramer⁺CD4⁺ T cells in spleen, blood, cervical and inguinal LNs and CNS of mice from FIG. 17A. Data correspond to 4 pMHC-NP-treated and 6 control-NP-treated mice. FIG. 17C shows changes in the average weights of HLA-DR4-IE-transgenic C57BL/6 IAb^{null} mice from FIG. 17A, immunized with hPLP₁₇₅₋₁₉₂ or hMOG₉₇₋₁₀₈ and treated with hPLP₁₇₅₋₁₉₂/DR4-IE-NPs, hMOG₉₇₋₁₀₈/DR4-IE-NPs or uncoated nanoparticles when the mice reached a score of 1.5. FIG. 17D shows LFB/H&E staining of the cerebellum of HLA-DR4-IE-transgenic C57BL/6 IAb^{null} mice from FIG. 17A showing reductions in inflammation and demyelination in mice treated with hPLP₁₇₅₋₁₉₂/DR4-IE or hMOG₉₇₋₁₀₈/DR4-IE-NPs compared with controls. FIG. 17E shows percentage of tetramer⁺CD4⁺ T cells in lymph nodes and bone marrow of the mice in FIG. 10A (C57BL/10.M HLA-DR4-IE mice immunized with bovine collagen) at the end of follow-up (10 doses, 5 weeks). FIG. 17F shows changes in the average weights of HLA-DR4-IE-transgenic C57BL/6 IAb^{null} mice immunized with hPLP₁₇₅₋₁₉₂ from FIG. 10F. FIG. 17G shows representative LFB/H&E staining of the cerebellum of HLA-DR4-IE-transgenic C57BL/6 IAb^{null} mice immunized with hPLP₁₇₅₋₁₉₂ and treated with hPLP₁₇₅₋₁₉₂/DR4-IE-NPs, hMOG₉₇₋₁₀₈/DR4-IE-NPs, hMOG₉₇₋₁₀₈ peptide i.v. or s.c. (8 μg per dose), hMOG₉₇₋₁₀₈/DR4-IE monomer (25 μg per dose), hMOG₉₇₋₁₀₈ peptide-NPs (using the molar equivalent of peptide delivered via pMHC-NPs; 0.68 μg per dose), or hMOG₉₇₋₁₀₈ peptide-MPs (15 μg peptide per dose) compared with mice left untreated or treated with uncoated NPs or MPs (at the same NP/MP number). FIG. 17H shows changes in the average EAE scores and body weights of HLA-DR4-IE-transgenic C57BL/6 IAb^{null} mice immunized with hPLP₁₇₅₋₁₉₂ in response to treatment with hMOG₉₇₋₁₀₈ peptide i.v. or s.c. (8 μg per dose16), hMOG₉₇₋₁₀₈/DR4-IE monomer (25 μg per dose), hMOG₉₇₋₁₀₈ peptide-NPs (0.68 μg peptide per dose), hMOG₉₇₋₁₀₈ peptide-MPs (15 μg peptide per dose (Getts, D. R. et al. (2012) Nature Biotechnol. 30:1217-1224)), or a single dose of hMOG₉₇₋₁₀₈ peptide-MPs (15 μg peptide (Getts, D. R. et al. (2012) Nature Biotechnol. 30:1217-1224)) compared with mice left untreated or treated with uncoated NPs or MPs (at the same NP/MP number) (n=4-5 per group). The cohort of mice treated with one dose had to be terminated after 2.5 weeks, owing to rapid progression of disease. FIG. 17I shows percentages of tetramer+CD4+ T cells in spleen, blood, cervical and inguinal LNs and bone marrow of mice from FIG. 17H (n=3-9 per group). Data were compared with Mann-Whitney U-test or two-way ANOVA. Data are averages±s.e.m.

FIGS. 18A-18Y show disease reversal by pMHC-NPs is driven by the T_{R}1 cytokines IL-21, IL-10 and TGF-β and involves several downstream cellular targets. FIG. 18A shows changes in blood glucose levels in diabetic NOD mice (>11 mM) treated with IGRP₄₋₂₂/IA^{g7}-NPs and blocking anti-IL-10, anti-IFNγ or anti-TGF-β mAbs or anti-HRPN rat-IgG (n=4-6 per group). FIGS. 18B and 18C show percentages of tetramer⁺CD4⁺ T cells in the spleens (FIG. 18B), and proliferation of CFSE-labelled 8.3-CD8⁺ T cells in the PLNs versus MLN of the mice from FIG. 11A at the end of follow up (FIG. 18C). FIG. 18D shows changes in blood glucose in hyperglycaemic NOD, NOD Il10^{-/-} and NOD Ifng^{-/-} mice (n=3-6 per group) in response to 2.5mi/IA^{g7}-NPs. FIGS. 18E and 18F show percentages of tetramer⁺CD4⁺ T cells in the spleens (FIG. 18E), and proliferation of CFSE-labelled 8.3-CD8⁺ T cells in the PLNs versus MLN of the mice from FIG. 18D at the end of follow up (FIG. 18F). FIG. 18G shows EAE scores of mice treated with pMHC-NPs and rat-IgG or blocking mAbs (n=4 per group). FIG. 18H shows LFB/H&E staining of the cerebellum of HLA-DR4-IE-transgenic C57BL/6 IAb^{null} mice from FIG. 18G, highlighting differences in inflammation and demyelination in mice treated with hPLP₁₇₅₋₁₉₂/DR4-IE-NPs and rat-IgG versus blocking anti-IL-10, anti-TGF-β or anti-IL-21R mAbs. FIG. 18I shows changes in the average body weights of HLA-DR4-IE-transgenic C57BL/6 IAb^{null} mice from FIG. 18G. FIG. 18J, Percentage of tetramer⁺CD4⁺ T cells in spleen, blood and inguinal LNs of mice from FIG. 18G (n=4 per group). FIGS. 18K and 18L show changes in the average EAE scores (FIG. 18K) and body weights (FIG. 18L) of C57BL/6 Il27r^{-/-} mice immunized with pMOG₃₅₋₅₅ and treated with pMOG₃₈₋₄₉/IA^{b}-NPs or uncoated nanoparticles starting on the day when mice reached a score of 1.5 (to synchronize the groups for disease activity) (n=7 and 4, respectively). FIG. 18M shows representative IBA1 and LFB/H&E stainings of the cerebellum and the corresponding relative rank scores of mice from FIG. 18K (n=3 and 4, respectively). FIG. 18N shows percentage of tetramer⁺CD4⁺ T cells in spleen, blood, inguinal LNs and bone marrow of mice from FIG. 18K (left), and representative CD49b and LAG-3 staining profiles of tetramer⁺ versus tetramer⁻ cells (right). FIG. 10 shows percentage of B220⁺ cells in the PLNs or MLNs of 2.5mi/IA^{g7}-NP- or HEL₁₄₋₂₂/IA^{g7}-NP-treated mice (n=4 per group). FIG. 18P shows correlation between the percentages of PLN and splenic B220⁺ cells and 2.5mi/IA^{g7} tetramer⁺CD4⁺ T cells in additional cohorts of mice treated with 2.5mi/IA^{g7}-NPs, over a range of total pMHC dose (0.75-25 μg of total pMHC) (n=24-28). FIG. 18Q, left, shows in vitro proliferation of CFSE-labelled BDC2.5 CD4⁺ T cells against 2.5mi or GPI₂₈₂₋₂₉₂ peptide-pulsed B cells purified from the PLNs or MLNs of untreated NOD mice or mice treated with 2.5mi/IA^{g7}-NPs (n=5-6 per group). Right, representative CFSE dilution profiles. Briefly, profiles show the extent of CFSE dilution in CFSE-labelled BDC2.5 CD4⁺ T cells cultured in the presence of 2.5mi or GPI₂₈₂₋₂₉₂ peptide-pulsed B cells purified from the PLNs or MLNs of untreated or 2.5mi/IA^{g7}-NP-treated NOD mice. FIG. 18R, PLN-derived B cells (105) from 2.5mi/IA^{g7}-NP-treated mice secrete IL-10 ex vivo in response to LPS (1 μg ml⁻¹). Data correspond to 6 pMHC-treated and 5 untreated NOD mice. FIGS. 18S and 18T, Changes in the percentages of 2.5mi (PKH26-labelled) compared with GPI₂₈₂₋₂₉₂ peptide-pulsed (CFSE-labelled) B cells (FIG. 18S) or DCs (FIG. 18T) 7 days after transfer (at 1:1 ratio) into untreated or 2.5mi/IA^{g7}-NP-treated NOD mice. Histograms show averaged ratios for each cell type and condition (n=3-4 mice per cell type and condition). FIG. 18U shows percentages of CD5⁺CD1d^{hi}eGFP⁺B220⁺ cells in mice treated as in FIG. 11B plus blocking Abs (n=4 each). FIG. 18V shows LPS-stimulated PLN B cells from NOD mice treated with 10 doses of 2.5mi/IA^{g7}-NPs suppress the proliferation of CFSE-labelled BDC2.5 CD4⁺ T cells by 2.5mi peptide-pulsed DCs in vitro, as compared to LPS-stimulated PLN B cells from untreated controls. FIG. 18X shows percentage of CD19⁺CD3⁻ cells in blood before and after 3 doses of 250 μg of anti-CD20 mAb (n=4). FIG. 18Y, 2.5 mi/IA^{g7}-NP-induced upregulation of IL-21 and IL-10 mRNA in memory eGFP⁻ BDC2.5 CD4⁺ T cells from BDC2.5-TCR-transgenic NOD Foxp3-eGFP donors in NOD Thy1^{a} hosts (n=5). P values were calculated by Pearson correlation, Mann-Whitney U-test or two-way ANOVA. Data are averages±s.e.m.

FIG. 19 shows effects of cytokine blockade or genetic deficiency on the cytokine profile of cognate CD4⁺ T cells expanded by 2.5mi/IA^{g7}-NPs. n=3 mice each. Data are averages±s.e.m.

FIGS. 20A and 20B show human T1D-relevant pMHCII-NPs, but not free peptide or peptide-coated nanoparticles or microparticles, expand cognate T_{R}1-like CD4⁺ T cells in human PBMC-engrafted NSG hosts. FIG. 20A shows FACS profiles (cognate versus control tetramer staining in hCD4⁺ T cells) of samples from mice identified as responders in Table 2. Numerical data on tetramer⁺ T cells are presented on Table 2. FIG. 20B shows representative FACS profiles (cognate versus control tetramer staining in splenic hCD4⁺ T cells) of human healthy control PBMC-engrafted NSG hosts treated with IGRP₁₃₋₂₅/DR3-NPs (left), or human T1D PBMC-engrafted NSG hosts treated with IGRP₁₃₋₂₅ peptide, IGRP₁₃₋₂₅ peptide-coated nanoparticles, IGRP₁₃₋₂₅ peptide-coated microparticles, or left untreated (right). See FIGS. 13A-13I legend for details.

FIG. 21 shows schematic of the proposed mode of operation of pMHCII-based nanomedicines. pMHCII-coated NPs (pMHC-NP, lacking costimulatory molecules) promote the differentiation of disease-primed (antigen-experienced) IFNγ-producing CD4⁺ T_{H}1-cells into memory T_{R}1-like CD4⁺ T cells followed by systemic expansion. This differentiation process (but not the subsequent expansion) requires both IFNγ and IL-10, whereas IL-27 is dispensable. The pMHC-NP-expanded (mono-specific) autoreactive T_{R}1-like CD4⁺ T cells then suppress other autoreactive T-cell responses by secreting IL-21, IL-10 and TGF-β, which act on local APCs (B cells, CD11c⁺ and CD11b⁺ cells) that have captured the cognate autoantigen and thus present cognate pMHCII complexes to the expanded T_{R}1-like cells. This interaction inhibits the proinflammatory function of the targeted APCs and blocks their ability to present other pMHC class I and class II complexes to non-pMHC-NP-cognate autoreactive T-cell specificities (note that the local APCs uptake both cognate and non-cognate autoantigens shed into the milieu simultaneously). Suppression of antigen-presentation requires IL-10 and TGF-β but not IFNγ or IL-21. Furthermore, cognate interactions between the pMHC-NP-expanded T_{R}1 CD4⁺ T cells and autoreactive B cells specific for the cognate autoantigen (able to display the cognate pMHCII complex on the surface) promotes their differentiation into B_{reg} cells in an IL-21-dependent manner, which contribute to promote local immunosuppression, likely by secreting IL-10. Suppression of antigen presentation selectively targets APCs displaying the cognate pMHC, but as local APCs that capture the cognate autoantigen also capture other autoantigens simultaneously, the autoregulatory CD4⁺ T cells expanded by pMHC-NPs blunt the presentation of other autoantigenic Pmhc complexes to a broad range of autoreactive T cells. This suppression is disease-specific and self-limiting.

FIGS. 22A-22C show autoregulatory T-cell expansion properties of pMHC class I and class II-coated PF-M NPs in vivo as a function of pMHC density and dose. (FIG. 22A) Percentages of 2.5mi/IA^{g7} tetramer+ cells in splenic CD4+ T-cells of 10 wk-old NOD mice treated with 10 doses (given over 5 wk) of preparations of 2.5mi/IA^{g7}-PF-M displaying different pMHC valencies. The x axis values correspond to the amounts of pMHC (in ug) given in each dose. Data correspond to net values of tetramer+ cells after subtraction of staining with a negative control tetramer (HEL₁₄₋₂₂/IA^{g7}). (FIG. 22B) T_{R}1 CD4⁺ Treg expansion potency of 10 doses of 2.5mi/IA^{g7}-PF-M vs. 2.5mi/IA^{g7}-SFP-Z NPs given over 5 wk. Data correspond to preparations carrying 22-45 pMHCs/NP. (FIG. 22C) Percentage increase in the mean fluorescence intensity of the TR1 cell marker CD49b on 2.5mi/IA^{g7} tetramer-positive cells expanded in vivo by different 2.5mi/IA^{g7}-NP preparations as a function of pMHC density. Such relationship did not exist when CD49b upregulation levels were plotted as a function of pMHC dose.

### BRIEF DESCRIPTION OF THE TABLES

Table 1. Functionalized PEG linkers.
Table 2. Codons.
Tables 3A and 3B. Transcriptional profile of pMHC-NP-expanded CD4+ T-cells. (A) QRT-PCR for a panel of 384 immunological markers in 2.5mi/IA^{g7} tetramer+versus tetramer− CD4+ T-cells sorted from NOD mice treated with 10 doses of 2.5mi/IA^{g7}-NPs from 10-15 wk of age (n=3 and 4 samples, respectively). The cells were stimulated in vitro with anti-CD3/anti-CD28 mAb-coated dynabeads before RNA collection. Panel summarizes the most significant differences. (B) QRT-PCR for 8 TR1-relevant markers, including markers that were not represented in the primer set used in (A). Data correspond to four additional 2.5mi/IA^{g7} tetramer+ and seven tetramer− CD4+ T-cell samples.
Table 4A, 4B, and 4C. Human T1D donors and outcome of pMHC-NP therapy in PBMC-engrafted NSG hosts.
Table 5 is an exemplary list of cancer-relevant antigens for use in this disclosure.
Table 6 is an exemplary list of diabetes-relevant antigens for use in this disclosure.
Table 7 is an exemplary list of multiple sclerosis-relevant antigens for use in this disclosure.
Table 8 is an exemplary list of Celiac Disease-relevant antigens for use in this disclosure.
Table 9 is an exemplary list of primary biliary cirrhosis-relevant antigens for use in this disclosure.
Table 10 is an exemplary list of pemphigus folliaceus-relevant antigens and pemphigus vulgaris-relevant antigens for use in this disclosure.
Table 11 is an exemplary list of neuromyelitis optica spectrum disorder-relevant antigens for use in this disclosure.
Table 12 is an exemplary list of allergic asthma-relevant antigens for use in this disclosure.
Table 13 is an exemplary list of inflammatory bowel disease-relevant antigens for use in this disclosure.
Table 14 is an exemplary list of systemic lupus erythematosus-relevant antigens for use in this disclosure.
Table 15 is an exemplary list of atherosclerosis-relevant antigens for use in this disclosure.
Table 16 is an exemplary list of chronic obstructive pulmonary disease-relevant antigens and emphysema-relevant antigens for use in this disclosure.
Table 17 is an exemplary list of psoriasis-relevant antigens for use in this disclosure.
Table 18 is an exemplary list of autoimmune hepatitis-relevant antigens for use in this disclosure.
Table 19 is an exemplary list of uveitis-relevant antigens for use in this disclosure.
Table 20 is an exemplary list of Sjogren Syndrome-relevant antigens for use in this disclosure.
Table 21 is an exemplary list of scleroderma-relevant antigens for use in this disclosure.
Table 22 is an exemplary list of anti-phospholipid syndrome-relevant antigens for use in this disclosure.
Table 23 is an exemplary list of ANCA-associated vasculitis-relevant antigens for use in this disclosure.
Table 24 is an exemplary list of Stiff Man Syndrome-relevant antigens for use in this disclosure.

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “an excipient” includes a plurality of excipients. The term “at least one” intends one or more.

Throughout this application, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 10%, 5%, or 1%.

### Definitions

Unless defined otherwise, all technical and scientific terms used herein have the same meaning as commonly understood by one of ordinary skill in the art to which this disclosure belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating or preventing multiple sclerosis. “Consisting of” shall mean excluding more than trace elements of other ingredients and substantial method steps. Embodiments defined by each of these transition terms are within the scope of this disclosure.

The terms “inhibiting,” “reducing,” or “prevention,” or any variation of these terms, when used in the claims and/or the specification includes any measurable decrease or complete inhibition to achieve a desired result.

By “biocompatible”, it is meant that the components of the delivery system will not cause tissue injury or injury to the human biological system. To impart biocompatibility, polymers and excipients that have had history of safe use in humans or with GRAS (Generally Accepted As Safe) status, will be used preferentially. By biocompatibility, it is meant that the ingredients and excipients used in the composition will ultimately be “bioabsorbed” or cleared by the body with no adverse effects to the body. For a composition to be biocompatible, and be regarded as non-toxic, it must not cause toxicity to cells. Similarly, the term “bioabsorbable” refers to nanoparticles made from materials that undergo bioabsorption in vivo over a period of time such that long term accumulation of the material in the patient is avoided. In a certain embodiment, the biocompatible nanoparticle is bioabsorbed over a period of less than 2 years, preferably less than 1 year and even more preferably less than 6 months. The rate of bioabsorption is related to the size of the particle, the material used, and other factors well recognized by the skilled artisan. A mixture of bioabsorbable, biocompatible materials can be used to form the nanoparticles used in this disclosure. In one embodiment, iron oxide and a biocompatible, bioabsorbable polymer can be combined. For example, iron oxide and PGLA can be combined to form a nanoparticle.

The term “dendrimer,” as used herein, refers to a repetitively branched molecule also referred to as an arborol or cascade molecule. With regards to nanoparticle synthesis, the term “dendrimer core” refers to the use of the dendrimer as the central component of a nanoparticle such that it forms the basis of the nanoparticle structure. In some embodiments, the nanoparticle core disclosed herein comprises a dendrimer.

The term “polymeric micelle,” as used herein, refers to an amphilic structure that comprises a hydrophobic core and a hydrophilic shell which can be prepared from block copolymers. With regards to nanoparticle synthesis, the term “polymeric micelle core refers to the use of the polymeric micelle as the central component of a nanoparticle such that it forms the basis of the nanoparticle structure. In some embodiments, the nanoparticle core disclosed herein comprises a polymeric micelle.

An antigen-MHC-nanoparticle complex (“NP-complex” or “complex” or pMHC-NP or “nanoparticle complex”) refers to presentation of a peptide, carbohydrate, lipid, or other antigenic segment, fragment, or epitope of an antigenic molecule or protein (i.e., self-peptide or autoantigen) on a surface, such as a nanoparticle core.

The “nanoparticle core” is the nanoparticle substrate that does or does not include layers or coatings. The nanoparticle complex comprises the core with at least the antigen-MHC complex coupled to the core.

“Density” when referring to pMHC per nanoparticle is calculated as the surface area of the nanoparticle core with or without outer layers, that can also include linkers. Surface area is the total available surface area of the construct used. In one aspect, when a PEG linker is used, this can increase the total diameter of the nanoparticle core by about 20 nm² of the nanoparticle which increases the surface area accordingly of the total available surface area of the nanoparticle. In other words, it is the final surface area of the nanoparticle without the addition of one or more of the pMHC, costimulatory molecules and/or cytokines.

“Antigen” as used herein refers to all, part, fragment, or segment of a molecule that can induce an immune response in a subject or an expansion of an immune cell, preferably a T or B cell.

The term “alkyl” refers to monovalent saturated aliphatic hydrocarbyl groups having from 1 to 10 carbon atoms (i.e., C₁-C₁₀ alkyl) or 1 to 6 carbon atoms (i.e., C₁-C₆ alkyl), or 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “alkoxy” refers to —O-alkyl.

A “mimic” is an analog of a given ligand or peptide, wherein the analog is substantially similar to the ligand. “Substantially similar” means that the analog has a binding profile similar to the ligand except the mimic has one or more functional groups or modifications that collectively accounts for less than about 50%, less than about 40%, less than about 30%, less than about 20%, less than about 10%, or less than about 5% of the molecular weight of the ligand.

“Immune cells” includes, e.g., white blood cells (leukocytes) that are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). As used herein, the term “B cell,” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface. As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3, including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses.

The term “effector T cells”, as used herein, refers to T cells that can specifically bind an antigen and mediate an immune response (effector function) without the need for further differentiation. Examples of effector T cells include CTLs, TH1 cells, TH2 cells, effector memory cells and T helper cells. In contrast to effector T cells, naïve T cells have not encountered their specific antigen:MHC complex, nor responded to it by proliferation and differentiation into an effector T cell. Effector T cells can be resting (in the GO phase of the cell cycle) or activated (proliferating).

The term “anti-pathogenic autoreactive T cell” refers to a T cell with anti-pathogenic properties (i.e., T cells that counteract an autoimmune disease such as MS, a MS-related disease or disorder, or pre-diabetes). These T cells can include anti-inflammatory T cells, central memory T cells, effector memory T cells, memory T cells, low-avidity T cells, T helper cells, autoregulatory T cells, cytotoxic T cells, natural killer T cells, regulatory T cells, TR1 cells, suppressor T cells, CD4+ T cells, CD8+ T cells and the like.

The term “anti-inflammatory T cell” refers to a T cell that promotes an anti-inflammatory response. The anti-inflammatory function of the T cell may be accomplished through production and/or secretion of anti-inflammatory proteins, cytokines, chemokines, and the like. Anti-inflammatory proteins are also intended to encompass anti-proliferative signals that suppress immune responses. Anti-inflammatory proteins include IL-4, IL-10, IL-13, IL-21, IL-23, IL-27, IFN-α, TGF-β, IL-Ira, G-CSF, and soluble receptors for TNF and IL-6.

The term “differentiated” refers to when a cell of a first type is induced into developing into a cell of a second type. In some embodiments, a cognate T cell is differentiated into a regulatory T_{R}1 cell. In some embodiments, an activated T cell is differentiated into a T_{R}1 cell. In some embodiments, a memory T cell is differentiated into a T_{R}1 cell. In some embodiments, a B cell is differentiated into a regulatory B cell.

As used herein, “knob-in-hole” refers to a polypeptidyl architecture requiring a protuberance (or “knob”) at an interface of a first polypeptide and a corresponding cavity (or a “hole”) at an interface of a second polypeptide, such that the protuberance can be positioned in the cavity so as to promote heteromultimer formation. Protuberances are constructed by replacing small amino acid side chains from the interface of the first polypeptide with larger side chains (e.g., phenylalanine or tyrosine). Cavities of identical or similar size to the protuberances are created in the interface of the second polypeptide by replacing large amino acid side chains with smaller ones (e.g., alanine or threonine). The protuberances and cavities can be made by synthetic means such as by altering the nucleic acid encoding the polypeptides or by peptide synthesis, using routine methods by one skilled in the art. In some embodiments, the interface of the first polypeptide is located on an Fc domain in the first polypeptide; and the interface of the second polypeptide is located on an Fc domain on the second polypeptide. Knob-in-hole heteromultimers and methods of their preparation and use are disclosed in U.S. Pat. Nos. 5,731,168; 5,807,706; 5,821,333; 7,642,228; 7,695,936; 8,216,805; and 8,679,785, all of which are incorporated by reference herein in their entirety

As used herein, “MHC-alpha-Fc/MHC-beta-Fc” refers to heterodimer comprising a first polypeptide and a second polypeptide, wherein the first polypeptide comprises an MHC class II α-chain and an antibody Fc domain; the second polypeptide comprises an MHC class II β-chain and an antibody Fc domain. A knob-in-hole MHC-alpha-Fc/MHC-beta-Fc further requires that the Fc domains of each polypeptide interface with one another through the complementary positioning of a protuberance on one Fc domain within the corresponding cavity on the other Fc domain.

The term “isolated” means separated from constituents, cellular and otherwise, in which the polynucleotide, peptide, polypeptide, protein, antibody, or fragment(s) thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragment(s) thereof, does not require “isolation” to distinguish it from its naturally occurring counterpart. In addition, a “concentrated”, “separated” or “diluted” polynucleotide, peptide, polypeptide, protein, antibody, or fragment(s) thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragment(s) thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as its glycosylation pattern. A mammalian cell, such as T-cell, is isolated if it is removed from the anatomical site from which it is found in an organism.

An “auto-reactive T cell” is a T cell that recognizes an “auto-antigen”, which is a molecule produced and contained by the same individual that contains the T cell.

A “pathogenic T cell” is a T cell that is harmful to a subject containing the T cell. Whereas, a non-pathogenic T cell is not substantially harmful to a subject, and an anti-pathogenic T cells reduces, ameliorates, inhibits, or negates the harm of a pathogenic T cell.

As used herein, the terms regulatory B-cells or B-regulatory cells (“B-regs”) intend those cells that are responsible for the anti-inflammatory effect, that is characterized by the expression of CD1d, CD5 and the secretion of IL-10. B-regs are also identified by expression of Tim-1 and can be induced through Tim-1 ligation to promote tolerance. The ability of being B-regs was shown to be driven by many stimulatory factors such as toll-like receptors, CD40-ligand and others. However, full characterization of B-regs is ongoing. B-regs also express high levels of CD25, CD86, and TGF-β. This subset of B cells is able to suppress Th1 proliferation, thus contributing to the maintenance of self-tolerance. The potentiation of B-reg function should become the aim of many immunomodulatory drugs, contributing to a better control of autoimmune diseases. See for example: ncbi.nlm.nih.gov/pubmed/23707422, last accessed on Oct. 31, 2013.

Type-1 T Regulatory (T_{R}1) cells are a subset of CD4+ T cells that have regulatory properties and are able to suppress antigen-specific immune responses in vitro and in vivo. These T_{R}1 cells are defined by their unique profile of cytokine production and make high levels of IL-10 and TGF-beta, but no IL-4 or IL-2. The IL-10 and TGF-beta produced by these cells mediate the inhibition of primary naive T cells in vitro. There is also evidence that T_{R} cells exist in vivo, and the presence of high IL-10-producing CD4(+) T cells in patients with severe combined immunodeficiency who have received allogeneic stem-cell transplants have been documented. T_{R}1 cells are involved in the regulation of peripheral tolerance and they could potentially be used as a cellular therapy to modulate immune responses in vivo. See for example: ncbi.nlm.nih.gov/pubmed/10887343, last accessed on Oct. 31, 2013.

T_{R}1 cells are defined by their ability to produce high levels of IL-10 and TGF-beta. Tr1 cells specific for a variety of antigens arise in vivo, but may also differentiate from naive CD4+ T cells in the presence of IL-10 in vitro. T_{R}1 cells have a low proliferative capacity, which can be overcome by IL-15. T_{R}1 cells suppress naive and memory T helper type 1 or 2 responses via production of IL-10 and TGF-beta. Further characterization of T_{R}1 cells at the molecular level will define their mechanisms of action and clarify their relationship with other subsets of Tr cells. The use of T_{R}1 cells to identify novel targets for the development of new therapeutic agents, and as a cellular therapy to modulate peripheral tolerance, can be foreseen. See for example, ncbi.nlm.nih.gov/pubmed/11722624, last accessed on Oct. 31, 2013.

An “an effective amount” is an amount sufficient to achieve the intended purpose, non-limiting examples of such include: initiation of the immune response, modulation of the immune response, suppression of an inflammatory response and modulation of T cell activity or T cell populations. In one aspect, the effective amount is one that functions to achieve a stated therapeutic purpose, e.g., a therapeutically effective amount. As described herein in detail, the effective amount, or dosage, depends on the purpose and the composition, and can be determined according to the present disclosure.

An effective amount of therapeutic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

An “MHC multimer” as the term is used herein means a complex of two or more, usually four, up to about fifty or more MHC monomers.

As used herein, a “multimer complex” refers to a complex between a target cell population and one or more pMHC complexes, wherein the MHC protein of the pMHC complex comprises multimeric form of the MHC protein. In some embodiments, the multimeric form of the MHC protein includes a dimer or a trimer.

As used herein, the phrase “immune response” or its equivalent “immunological response” refers to the development of a cell-mediated response (mediated by antigen-specific T cells or their secretion products). A cellular immune response is elicited by the presentation of polypeptide epitopes in association with Class I or Class II MHC molecules, to treat or prevent a viral infection, expand antigen-specific Breg cells, TC1, CD4⁺ T helper cells and/or CD8+ cytotoxic T cells and/or disease generated, autoregulatory T cell and B cell “memory” cells. The response may also involve activation of other components. In some aspects, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, such as but not limited to, B cells or antigen-presenting cells—non limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells.

By “nanosphere,” “NP,” or “nanoparticle” herein is meant a small discrete particle that is administered singularly or pluraly to a subject, cell specimen or tissue specimen as appropriate. In certain embodiments, the term “nanoparticle” as used herein includes any layers around the nanoparticle core. In certain embodiments, the nanoparticles are substantially spherical in shape. In certain embodiments, the nanoparticle is not a liposome or a viral particle. In further embodiments, the nanoparticle is comprised of any appropriate material, e.g., a solid, a solid core, a metal, a dendrimer, a polymeric micelle, a metal oxide, or a protein or fragment or combinations thereof. The term “substantially spherical,” as used herein, means that the shape of the particles does not deviate from a sphere by more than about 10%. Various known antigen or peptide complexes of the disclosure may be applied to the particles. The nanoparticles of this disclosure range in size from about 1 nm to about 1 μm and, preferably, from about 1 nm to about 500 nm or alternatively from about 1 nm to about 100 nm, or alternatively from about 1 nm to about 50 nm or alternatively from about 5 nm to about 100 nm, and in some aspects refers to the average or median diameter of a plurality of nanoparticles when a plurality of nanoparticles are intended. Smaller nanosize particles can be obtained, for example, by the process of fractionation whereby the larger particles are allowed to settle in an aqueous solution. The upper portion of the solution is then recovered by methods known to those of skill in the art. This upper portion is enriched in smaller size particles. The process can be repeated until a desired average size is generated. The term “nanostructure” is used generally to describe structures smaller than about 1 μm.

The terms “inflammatory response” and “inflammation” as used herein indicate the complex biological response of vascular tissues of an individual to harmful stimuli, such as pathogens, damaged cells, or irritants, and includes secretion of cytokines and, more particularly, of pro-inflammatory cytokines, i.e. cytokines which are produced predominantly by activated immune cells and are involved in the amplification of inflammatory reactions. Exemplary pro-inflammatory cytokines include but are not limited to IL-1, IL-6, IL-10, TNF-α, IL-17, IL21, IL23, IL27 and TGF-β. Exemplary inflammations include acute inflammation and chronic inflammation. Acute inflammation indicates a short-term process characterized by the classic signs of inflammation (swelling, redness, pain, heat, and loss of function) due to the infiltration of the tissues by plasma and leukocytes. An acute inflammation typically occurs as long as the injurious stimulus is present and ceases once the stimulus has been removed, broken down, or walled off by scarring (fibrosis). Chronic inflammation indicates a condition characterized by concurrent active inflammation, tissue destruction, and attempts at repair. Chronic inflammation is not characterized by the classic signs of acute inflammation listed above. Instead, chronically inflamed tissue is characterized by the infiltration of mononuclear immune cells (monocytes, macrophages, lymphocytes, and plasma cells), tissue destruction, and attempts at healing, which include angiogenesis and fibrosis. An inflammation can be inhibited in the sense of the present disclosure by affecting and in particular inhibiting any one of the events that form the complex biological response associated with an inflammation in an individual.

As used herein, “CD49b” or “cluster of differentiation 49b” is a protein that is an integrin alpha subunit and makes up about half of the alpha2beta1 integrin duplex. In humans, CD49b is encoded by the CD49 b gene. CD49b can be found on a wide variety of cell types, including T cells, natural killer cells, fibroblasts, and platelets. In some embodiments, the T cell includes a T_{R}1 cell. In some embodiments, the expression of CD49b identifies a T_{R}1 cell. Detection of a cell expressing CD49b can be identified using conventional techniques, such as the use of an anti-CD49b antibody, which are commercially available, e.g., from a vendor such as BioLegend.

As used herein, “Lag3” or “lymphocyte-activation gene 3” or “CD223” or “cluster of differentiation 223” is a protein that is encoded by the Lag3 gene and belongs to the immunoglobulin (Ig) superfamily. Lag 3 is a cell surface protein that is expressed in a variety of cell types, including T cells, natural killer cells, B cells, and plasmacytoid dendritic cells. In some embodiments, the T cell includes a T_{R}1 cell. In some embodiments, the expression of Lag3 identifies a T_{R}1 cell. Detection of a cell expressing Lag3 can be identified using conventional techniques, such as the use of an anti-Lag3 antibody, which are commercially available, e.g., from a vendor such as BioLegend.

As used herein, the term “disease-relevant” antigen intends an antigen or fragment thereof selected to treat a selected disease and is involved in the disease process. For example, a diabetes-relevant antigen is an antigen or fragment thereof that, when presented, produces an immune response that serves to treat diabetes; thus, a diabetes-relevant antigen producing such an effect is selected to treat diabetes. A multiple sclerosis (MS)-relevant antigen is selected to treat MS. A diabetes-relevant antigen would not be selected to treat MS. Similarly, an autoimmunity-related antigen is an antigen that is relevant to an autoimmune disease and would not be selected for the treatment of a disorder or disease other than autoimmunity, e.g., cancer. Non-limiting, exemplary disease-relevant antigens are disclosed herein and further, such antigens may be determined for a particular disease based on techniques, mechanisms, and methods documented in the literature.

“Autoimmune disease or disorder” includes diseases or disorders arising from and directed against an individual's own tissues or organs or manifestation thereof or a condition resulting there from. In one embodiment, it refers to a condition that results from, or is aggravated by, the production by T cells that are reactive with normal body tissues and antigens. Examples of autoimmune diseases or disorders include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica spectrum disorder (NMO, also known as Devic's Disease or Devic's Syndrome), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, Type I diabetes, Type II diabetes, latent autoimmune diabetes in adults (or Type 1.5 diabetes) Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, anti-phospholipid syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, acquired thrombocytopenic purpura, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, emphysema, alopecia areata, adipose tissue inflammation/diabetes type II, obesity associated adipose tissue inflammation/insulin resistance, and endometriosis.

In some embodiments, the autoimmune disorder or disease may include, but is not limited to, diabetes melitus Type I and Type II, pre-diabetes, transplantation rejection, multiple sclerosis, a multiple-sclerosis related disorder, premature ovarian failure, scleroderma, Sjogren's disease/syndrome, lupus, vitiligo, alopecia (baldness), polyglandular failure, Grave's disease, hypothyroidism, polymyosititis, pemphigus, Crohn's disease, colitis, autoimmune hepatitis, hypopituitarism, myocarditis, Addison's disease, autoimmune skin diseases, uveitis, pernicious anemia, hypoparathyroidism, and/or rheumatoid arthritis. Other indications of interest include, but are not limited to, asthma, allergic asthma, primary biliary cirrhosis, cirrhosis, Neuromyelitis Optica Spectrum Disorder (Devic's disease, opticospinal multiple scleroris (OSMS)), Pemphigus vulgaris, inflammatory bowel disease (IBD), arthritis, Rheumatoid arthritis, systemic lupus erythematosus (SLE), Celiac disease, psoriasis, autoimmune cardiomyopathy, idiopathic dilated cardiomyopathy (IDCM), a Myasthyenia Gravis, Uveitis, Ankylosing Spondylitis, Immune Mediated Myopathies, prostate cancer, anti-phospholipid syndrome (ANCA+), atherosclerosis, dermatomyositis, chronic obstructive pulmonary disease (COPD), emphysema, spinal cord injury, traumatic injury, a tobacco-induced lung destruction, ANCA-associated vasculitis, psoriasis, sclerosing cholangitis, primary sclerosing cholangitis, and diseases of the central and peripheral nervous systems.

In some embodiments, the autoimmune disorder or disease may include, but is not limited to, diabetes, multiple sclerosis, Celiac Disease, primary biliary cirrhosis, pemphigus, pemphigus folliaceus, pemphigus vulgaris, neuromyelitis optica spectrum disorder, arthritis (including rheumatoid arthritis), allergic asthma, inflammatory bowel disease (including Crohn's disease and ulcerative colitis), systemic lupus erythematosus, atherosclerosis, chronic obstructive pulmonary disease, emphysema, psoriasis, autoimmune hepatitis, uveitis, Sjogren's Syndrome, scleroderma, anti-phospholipid syndrome, ANCA-associated vasculitis, and Stiff Man Syndrome.

As used herein, the term “adipose tissue inflammation/diabetes type II” refers to the adipose tissue inflammation exhibited by a subject suffering from type II diabetes. The adipose tissue inflammation contributes to the development of insulin resistance in the subject.

As used herein, the term “obesity associated adipose tissue inflammation/insulin resistance” refers to the adipose tissue inflammation exhibited by a subject suffering from obesity. The adipose tissue inflammation contributes to the insulin resistance of the subject, thereby increasing the likelihood that the adipose tissue inflammation will result in the pathogensis of type II diabetes.

As used herein, the term “canonical sequence” refers to the protein sequence used as a reference for amino acid numbering in the absence of further guidance in the disclosure or the existing art. As is apparent to those of skill in the art, the termini of the antigenic fragments may vary with the reference sequence from which the fragment has been mapped to. Thus, it is to be understood unless specifically stated otherwise that the fragment identifiers are approximate termini.

As used herein, “PPI” refers to all isoforms, variants, and fragments thereof of a protein associated with the name “preproinsulin,” a biologically inactive precursor to the biologically active endocrine hormone insulin, or a biological equivalent thereof. The canonical sequence of the isoform PPI is 110 amino acids in length:

MALWMRLLPL LALLALWGPD PAAAFVNQHL CGSHLVEALY
LVCGERGFFY TPKTRREAED LQVGQVELGG GPGAGSLQPL
ALEGSLQKRG IVEQCCTSIC SLYQLENYCN.

As used herein, “IGRP” refers to all isoforms, variants, and fragments thereof of a protein associated with the name “islet-specific glucose-6-phosphatase catalytic subunit-related protein” or “Glucose-6-phosphatase-2” a major autoantigen for autoimmune type 1 diabetes, or a biological equivalent thereof. The canonical sequence of IGRP is 355 amino acids in length:

MDFLHRNGVLIIQHLQKDYRAYYTFLNFMSNVGDPRNIFFIYFPLCFQFN
QTVGTKMIWVAVIGDWLNLIFKWILFGHRPYWWVQETQIYPNHSSPCLEQ
FPTTCETGPGSPSGHAMGASCVWYVMVTAALSHTVCGMDKFSITLHRLTW
SFLWSVFWLIQISVCISRVFIATHFPHQVILGVIGGMLVAEAFEHTPGIQ
TASLGTYLKTNLFLFLFAVGFYLLLRVLNIDLLWSVPIAKKWCANPDWIH
IDTTPFAGLVRNLGVLFGLGFAINSEMFLLSCRGGNNYTLSFRLLCALTS
LTILQLYHFLQIPTHEEHLFYVLSFCKSASIPLTVVAFIPYSVHMLMKQS
GKKSQ.

As used herein, “GAD” refers to all isoforms, variants, and fragments thereof of a protein associated with the name “glutamic acid decarboxylase” a diabetes-associated antigen, or a biological equivalent thereof. GAD may optionally refer to GAD1, GAD2, GAD65, GAD67, or any other diabetes relevant glutamic acid decarboxylase. The canonical sequence of the isoform GAD2 is 585 amino acids in length and is disclosed herein below:

MASPGSGFWS FGSEDGSGDS ENPGTARAWC QVAQKFTGGI
GNKLCALLYG DAEKPAESGGSQPPRAAARK AACACDQKPC
SCSKVDVNYA FLHATDLLPA CDGERPTLAF
LQDVMNILLQYVVKSFDRST KVIDFHYPNE LLQEYNWELA
DQPQNLEEIL MHCQTTLKYA IKTGHPRYFNQLSTGLDMVG
LAADWLTSTA NTNMFTYEIA PVFVLLEYVT LKKMREIIGW
PGGSGDGIFSPGGAISNMYA MMIARFKMFP EVKEKGMAAL
PRLIAFTSEH SHFSLKKGAA ALGIGTDSVILIKCDERGKM
IPSDLERRIL EAKQKGFVPF LVSATAGTTV YGAFDPLLAV
ADICKKYKIWMHVDAAWGGG LLMSRKHKWK LSGVERANSV
TWNPHKMMGV PLQCSALLVR EEGLMQNCNQMHASYLFQQD
KHYDLSYDTG DKALQCGRHV DVFKLWLMWR AKGTTGFEAH
VDKCLELAEYLYNIIKNREG YEMVFDGKPQ HTNVCFWYIP
PSLRTLEDNE ERMSRLSKVA PVIKARMMEYGTTMVSYQPL
GDKVNFFRMV ISNPAATHQD IDFLIEEIER LGQDL.

As used herein “peripherin” refers to all isoforms, variants, and fragments thereof of a protein associated with that name, or a biological equivalent thereof. A non-limiting exemplary sequence of human peripherin associated with UniProt Reference No. P41219 is disclosed herein below:

MSHHPSGLRAGFSSTSYRRTFGPPPSLSPGAFSYSSSSRFSSSRLLGSAS
PSSSVRLGSFRSPRAGAGALLRLPSERLDFSMAEALNQEFLATRSNEKQE
LQELNDRFANFIEKVRFLEQQNAALRGELSQARGQEPARADQLCQQELRE
LRRELELLGRERDRVQVERDGLAEDLAALKQRLEEETRKREDAEHNLVLF
RKDVDDATLSRLELERKIESLMDEIEFLKKLHEEELRDLQVSVESQQVQQ
VEVEATVKPELTAALRDIRAQYESIAAKNLQEAEEWYKSKYADLSDAANR
NHEALRQAKQEMNESRRQIQSLTCEVDGLRGTNEALLRQLRELEEQFALE
AGGYQAGAARLEEELRQLKEEMARHLREYQELLNVKMALDIEIATYRKLL
EGEESRISVPVHSFASLNIKTTVPEVEPPQDSHSRKTVLIKTIETRNGEV
VTESQKEQRSELDKSSAHSY.

As used herein, “aGlia” refers to all isoforms, variants, and fragments thereof of a protein associated with the name “Alpha/beta-gliadin,” derived from a member of the wheat family or another celiac-related allergen, or a biological equivalent thereof. A non-limiting exemplary sequence of alpha-gliadin expressed in wheat associated with GenBank Accession No. CAA10257.1 is:

MKTFLILALLAIVATTATTAVRVPVPQLQPQNPSQQQPQEQVPLVQQQQF
LGQQQPFPPQQPYPQPQPFPSQQPYLQLQPFPQPQLPYSQPQPFRPQQPY
PQPQPQYSQPQQPISQQQQQQQQQQQQQQQQQQQQILQQILQQQLIPCMD
VVLQQHNIAHGRSQVLQQSTYQLLQELCCQHLWQIPEQSQCQAIHKVVHA
IILHQQQKQQQQPSSQVSFQQPLQQYPLGQGSFRPSQQNPQAQGSVQPQQ
LPQFEEIRNLALQTLPAMCNVYIPPYCTITPFGIFGTN.

Another non-limiting exemplary sequence of alpha-gliadin expressed in wheat is disclosed herein below:

VRVPVPQLQPQNPSQQQPQEQVPLVQQQQFLGQQQPFPPQQPYPQPQPFP
SQQPYLQLQPFPQPQLPYSQPQPFRPQQPYPQPQPQYSQPQQPISQQQQQ
QQQQQQQQQQQQQQQILQQILQQQLIPCMDVVLQQHNIAHGRSQVLQQST
YQLLQELCCQHLWQIPEQSQCQAIHKVVHAIILHQQQKQQQQPSSQVSFQ
QPLQQYPLGQGSFRPSQQNPQAQGSVQPQQLPQFEEIRNLALQTLPAMCN
VYIPPYCTITPFGIFGTN.

As used herein, “PDC-E2” refers to all isoforms, variants, and fragments thereof of a protein associated with the name “dihydrolipoamide S-acetyltransferase” or “DLAT,” an autoantigen of primary biliary cirrhosis, or a biological equivalent thereof. The canonical sequence of PDC-E2 is 647 amino acids in length and is disclosed herein below:

MWRVCARRAQ NVAPWAGLEA RWTALQEVPG TPRVTSRSGP
APARRNSVTT GYGGVRALCGWTPSSGATPR NRLLLQLLGS
PGRRYYSLPP HQKVPLPSLS PTMQAGTIAR WEKKEGDKIN
EGDLIAEVET DKATVGFESL EECYMAKILV AEGTRDVPIG
AIICITVGKP EDIEAFKNYTLDSSAAPTPQ AAPAPTPAAT
ASPPTPSAQA PGSSYPPHMQ VLLPALSPTM
TMGTVQRWEKKVGEKLSEGD LLAEIETDKA TIGFEVQEEG
YLAKILVPEG TRDVPLGTPL CIIVEKEADISAFADYRPTE
VTDLKPQVPP PTPPPVAAVP PTPQPLAPTP SAPCPATPAG
PKGRVFVSPLAKKLAVEKGI DLTQVKGTGP DGRITKKDID
SFVPSKVAPA PAAVVPPTGP GMAPVPTGVFTDIPISNIRR
VIAQRLMQSK QTIPHYYLSI DVNMGEVLLV RKELNKILEG
RSKISVNDFIIKASALACLK VPEANSSWMD TVIRQNHVVD
VSVAVSTPAG LITPIVFNAH IKGVETIANDVVSLATKARE
GKLQPHEFQG GTFTISNLGM FGIKNFSAII NPPQACILAI
GASEDKLVPADNEKGFDVAS MMSVTLSCDH RVVDGAVGAQ
WLAEFRKYLE KPITMLL.

As used herein, “Insulin” refers to all isoforms, variants, and fragments thereof of a protein associated with that name, or a biological equivalent thereof. A non-limiting exemplary sequence of human insulin associated with UniProt Reference No. P01308 is disclosed herein below:

MALWMRLLPLLALLALWGPDPAAAFVNQHLCGSHLVEALYLVCGERGFFY
TPKTRREAEDLQVGQVELGGGPGAGSLQPLALEGSLQKRGIVEQCCTSIC
SLYQLENYCN.

As used herein, “DG1EC2” refers to all isoforms, variants, and fragments thereof of a protein associated with the name “desmosomal glycoprotein 1,” or a biological equivalent thereof. The canonical sequence of DG1EC2 is 1054 amino acids in length and is disclosed herein below:

MNWHFLRTAT VLLIFLVVVE INSEFRIQVR DYNTKNGTIK
WHSIRRQKRE WIKFAAACREGEDNSKRNPI AKIHSDCAAN
QQVTYRISGV GIDQPPYGIF IINQKTGEIN
ITSIVDREITPFFIIYCRAL NSLGQDLERP LELRVRVLDI
NDNPPVFSMS TFVGQIEENS NANTLVMRLNATGADEPNNL
NSKIAFKIIR QEPSDSPMFI INRNTGEIRT MNNFLDREQY
SQYSLAVRGSDRDGGADGMS AECECNIKIL DVNDNIPYME
PSSHMVRIEE NALSQNLVEI RVIDLDEEFSANWMAVIFFI
SGNEGGWFDI EMNERTNVGI LKVIKPLDYE AVQNLQLSLG
VRNKADFHHSIMSQYKVTAT AISVTVLNVI EGSVFRPGSK
TYVVRSDMGQ NYKVGDFVAT DLDTGLASTTVRYVMGNNPA
NLLNVDSKTG VITLRNKVTM EQYEMLNGKY QGTILSIDDA
LQRTCTGTINIDLQGSGWEK DSEKVTSSQN SGSSTGDSSG
GTGGGGRENP SEGDTTTNTG GKTSTDYEDGETQTQSNNNH
QELGSNNLSD NVHFGPAGIG LLIMGFLVLG LVPFLLMCCD
CGGAPGAGAGFEPVPECSDG AIHSWAVEGP QPLPTDATTV
CVPPIPSNNA NVIECIDTSG VYTNEYGGREMQDLGGGERT
TGFELTEGVK TSGVPEICQE YSGTLRRNSM RECREGGLNM
NFMESYFCQKAYAYADEDEG RPSNDCLLIY DIEGVGSPAG
SVGCCSFIGE DLDDSFLDTL GPKFKKLADISLGKEVEPDP
SWPPESTEPI CPQQGTEPII GGHPPISPHF GTTTVISENT
YPSGPGVQHPMPIPDPLGYG NVTVTESYTT SGTLKPTVHV
HDNRHASNVV VTERVVGPIS GTDLHGMLEMPDLRDGSNVI.

As used herein, “DG3” refers to all isoforms, variants, and fragments thereof of a protein associated with the name “desmoglein 3”, or a biological equivalent thereof. A non-limiting exemplary sequence of human desmoglein 3 associated with UniProt Reference No. P32926-1 is disclosed herein below:

MMGLFPRTTGALAIFVVVILVHGELRIETKGQYDEEEMTMQQAKRRQKRE
WVKFAKPCREGEDNSKRNPIAKITSDYQATQKITYRISGVGIDQPPFGIF
VVDKNTGDINITAIVDREETPSFLITCRALNAQGLDVEKPLILTVKILDI
NDNPPVFSQQIFMGEIEENSASNSLVMILNATDADEPNHLNSKIAFKIVS
QEPAGTPMFLLSRNTGEVRTLTNSLDREQASSYRLVVSGADKDGEGLSTQ
CECNIKVKDVNDNFPMFRDSQYSARIEENILSSELLRFQVTDLDEEYTDN
WLAVYFFTSGNEGNWFEIQTDPRTNEGILKVVKALDYEQLQSVKLSIAVK
NKAEFHQSVISRYRVQSTPVTIQVINVREGIAFRPASKTFTVQKGISSKK
LVDYILGTYQAIDEDTNKAASNVKYVMGRNDGGYLMIDSKTAEIKFVKNM
NRDSTFIVNKTITAEVLAIDEYTGKTSTGTVYVRVPDFNDNCPTAVLEKD
AVCSSSPSVVVSARTLNNRYTGPYTFALEDQPVKLPAVWSITTLNATSAL
LRAQEQIPPGVYHISLVLTDSQNNRCEMPRSLTLEVCQCDNRGICGTSYP
TTSPGTRYGRPHSGRLGPAAIGLLLLGLLLLLLAPLLLLTCDCGAGSTGG
VTGGFIPVPDGSEGTIHQWGIEGAHPEDKEITNICVPPVTANGADFMESS
EVCTNTYARGTAVEGTSGMEMTTKLGAATESGGAAGFATGTVSGAASGFG
AATGVGICSSGQSGTMRTRHSTGGTNKDYADGAISMNFLDSYFSQKAFAC
AEEDDGQEANDCLLIYDNEGADATGSPVGSVGCCSFIADDLDDSFLDSLG
PKFKKLAEISLGVDGEGKEVQPPSKDSGYGIESCGHPIEVQQTGFVKCQT
LSGSQGASALSTSGSVQPAVSIPDPLQHGNYLVTETYSASGSLVQPSTAG
FDPLLTQNVIVTERVICPISSVPGNLAGPTQLRGSHTMLCTEDPCSRLI.

As used herein, “AQP4” refers to all isoforms, variants, and fragments thereof of a protein associated with the name “aquaporin 4,” which belongs to the aquaporin family of integral membrane proteins that conduct water through the cell membrane and is the primary autoimmune target of neuromyelitis optica spectrum disorder, or a biological equivalent thereof. The canonical sequence of AQP4 is 323 amino acids in length and is disclosed herein below:

MSDRPTARRWGKCGPLCTRENIMVAFKGVWTQAFWKAVTAEFLAMLIFVL
LSLGSTINWGGTEKPLPVDMVLISLCFGLSIATMVQCFGHISGGHINPAV
TVAMVCTRKISIAKSVFYIAAQCLGAIIGAGILYLVTPPSVVGGLGVTMV
HGNLTAGHGLLVELIITFQLVFTIFASCDSKRTDVTGSIALAIGFSVAIG
HLFAINYTGASMNPARSFGPAVIMGNWENHWIYWVGPIIGAVLAGGLYEY
VFCPDVEFKRRFKEAFSKAAQQTKGSYMEVEDNRSQVETDDLILKPGVVH
VIDVDRGEEKKGKDQSGEVLSSV.

As used herein, “PLP” refers to all isoforms, variants, and fragments thereof of a protein associated with the name “myelin proteolipid protein”, or a biological equivalent thereof. A non-limiting exemplary sequence of human myelin proteolipid protein associated with UniProt Reference No. P60201 is disclosed herein below:

MGLLECCARCLVGAPFASLVATGLCFFGVALFCGCGHEALTGTEKLIETY
FSKNYQDYEYLINVIHAFQYVIYGTASFFFLYGALLLAEGFYTTGAVRQI
FGDYKTTICGKGLSATVTGGQKGRGSRGQHQAHSLERVCHCLGKWLGHPD
KFVGITYALTVVWLLVFACSAVPVYIYFNTWTTCQSIAFPSKTSASIGSL
CADARMYGVLPWNAFPGKVCGSNLLSICKTAEFQMTFHLFIAAFVGAAAT
LVSLLTFMIAATYNFAVLKLMGRGTKF.

As used herein, “MOG” refers to all isoforms, variants, and fragments thereof of a protein associated with the name “Myelin Oligodendrocyte Glycoprotein,” or a biological equivalent thereof. A non-limiting exemplary sequence of human myelin oligodendrocyte glycoprotein associated with UniProt Reference No. 016653 is disclosed herein below:

MASLSRPSLPSCLCSFLLLLLLQVSSSYAGQFRVIGPRHPIRALVGDEVE
LPCRISPGKNATGMEVGWYRPPFSRVVHLYRNGKDQDGDQAPEYRGRTEL
LKDAIGEGKVTLRIRNVRFSDEGGFTCFFRDHSYQEEAAMELKVEDPFYW
VSPGVLVLLAVLPVLLLQITVGLIFLCLQYRLRGKLRAEIENLHRTFDPH
FLRVPCWKITLFVIVPVLGPLVALIICYNWLHRRLAGQFLEELRNPF.

As used herein “MBP” refers to all isoforms, variants, and fragments thereof of a protein associated with the name “myelin basic protein”, or a biological equivalent thereof. A non-limiting exemplary sequence of human myelin basic protein associated with UniProt Reference No. P02686 is disclosed herein below:

MGNHAGKRELNAEKASTNSETNRGESEKKRNLGELSRTTSEDNEVFGEAD
ANQNNGTSSQDTAVTDSKRTADPKNAWQDAHPADPGSRPHLIRLFSRDAP
GREDNTFKDRPSESDELQTIQEDSAATSESLDVMASQKRPSQRHGSKYLA
TASTMDHARHGFLPRHRDTGILDSIGRFFGGDRGAPKRGSGKDSHHPART
AHYGSLPQKSHGRTQDENPVVHFFKNIVTPRTPPPSQGKGRGLSLSRFSW
GAEGQRPGFGYGGRASDYKSAHKGFKGVDAQGTLSKIFKLGGRDSRSGSP
MARR.

As used herein, “CII” refers to all isoforms, variants, and fragments thereof of a protein associated with the name “native collagen type II”, a high molecular-weight fibrillar molecule implicated in chronic polyarthritis, or a biological equivalent thereof. A non-limiting exemplary consensus sequence of human collagen II is disclosed herein below:

MRGASVTVAAVRCGDVAGSCVDGRYNDKDVWKCRCVCDTGTVCDDCDVKD
CSGCCCTDATASGGKGKGGDKDVGKGGGAGGRGDRGDKGKGAGRGRDGGT
GNGGGGGGGNAAMAGGDKAGGAGVMGMGMGRGGAGAGGGNGGGVSGMGRG
GGKGDDGAGKGKAGRGGGARGGTGGVKGHRGYGDGAKGAGAGVKGSGSGN
GSGMGRGGRGRTGAGAAGARGNDGGAGGVGAGGGGAGAKGAGTGARGGAG
RGGTGSGAGASGNGTDGGAKGSAGAGAGAGGRGGGATGGKGTGGAGKGGK
GGAGGAGAGGKRGARGGGVGGGRGAGNRGGDGAGKGAGRGSGAGKGANGD
GRGGGARGTGRGDAGGKVGSGAGDGRGGGARGGVMGGKGANGGKAGKGGA
GRGGKDGTGAAGGAGAGRGGAGSGGGGGGGKGDGVGAGAGVGRGRGGRGS
GAGGRGGTGTDGKGASGAGGAGGGMGRGAAGAGKGDRGDVGKGGAGKDGG
RGTGGGAGANGKGVGGAGSAGARGAGRGTGGAGAGGADGGAKGGAGKGDA
GAGGSGAGGTGVTGKGARGAGGATGGAAGRVGGSNGNGGGSGKDGKGARG
DSGGRAGGGAGGKGGDDGSGAGGGAGRGVGGRGRGGGSGGKGAGASGDRG
GVGGTGAGGRGSGADGGRDGAAGVKGDRGTGAVGAGAGGSGAGTGKGDRG
AGAGMGSGAGARGGGRGDKGAGGRGKGHRGTGGGGSGDGASGAGSGRGGV
GSGKDGANGGGGRGRSGTGAGGNGGGGGDMSAAGGRKGDYMRADAAGGRH
DAVDATKSNNSRSGSRKNARTCRDKCHWKSGDYWDNGCTDAMKVCNMTGT
CVYNANVKKNWWSSKSKKKHWGTNGGHSYGDDNANTANVMTRSTGSNTYH
CKNSAYDAAGNKKAGSNDVRAGNSRTYTAKDGCTKHTGKWGKTVYRSKTS
RDAMDGGGVDGVC.

Another non-limiting exemplary sequence of murine collagen II is disclosed herein below:

MIRLGAPQSL VLLTLLIAAV LRCQGQDARK LGPKGQKGEP
GDIRDIIGPR GPPGPQGPAGEQGPRGDRGD KGEKGAPGPR
GRDGEPGTPG NPGPAGPPGP PGPPGLSAGN
FAAQMAGGYDEKAGGAQMGV MQGPMGPMGP RGPPGPAGAP
GPQGFQGNPG EPGEPGVSGP MGPRGPPGPAGKPGDDGEAG
KPGKSGERGL PGPQGARGFP GTPGLPGVKG HRGYPGLDGA
KGEAGAPGVKGESGSPGENG SPGPMGPRGL PGERGRTGPA
GAAGARGNDG QPGPAGPPGP VGPAGGPGFP GAPGAKGEAG
PTGARGPEGA QGSRGEPGNP GSPGPAGASG NPGTDGIPGA
KGSAGAPGIAGAPGFPGPRG PPGPQGATGP LGPKGQAGEP
GIAGFKGDQG PKGETGPAGP QGAPGPAGEEGKRGARGEPG
GAGPIGPPGE RGAPGNRGFP GQDGLAGPKG APGERGPSGL
TGPKGANGDPGRPGEPGLPG ARGLTGRPGD AGPQGKVGPS
GAPGEDGRPG PPGPQGARGQ PGVMGFPGPKGANGEPGKAG
EKGLAGAPGL RGLPGKDGET GAAGPPGPSG PAGERGEQGA
PGPSGFQGLP GPPGPPGEGG KQGDQGIPGE AGAPGLVGPR
GERGFPGERG SPGAQGLQGP RGLPGTPGTDGPKGAAGPDG
PPGAQGPPGL QGMPGERGAA GIAGPKGDRG DVGEKGPEGA
PGKDGGRGLTGPIGPPGPAG ANGEKGEVGP PGPSGSTGAR
GAPGERGETG PPGPAGFAGP PGADGQPGAKGDQGEAGQKG
DAGAPGPQGP SGAPGPQGPT GVTGPKGARG AQGPPGATGF
PGAAGRVGPPGANGNPGPAG PPGPAGKDGP KGVRGDSGPP
GRAGDPGLQG PAGAPGEKGE PGDDGPSGLD GPPGPQGLAG
QRGIVGLPGQ RGERGFPGLP GPSGEPGKQG APGASGDRGP
PGPVGPPGLTGPAGEPGREG SPGADGPPGR DGAAGVKGDR
GETGALGAPG APGPPGSPGP AGPTGKQGDRGEAGAQGPMG
PSGPAGARGI AGPQGPRGDK GESGEQGERG LKGHRGFTGL
QGLPGPPGPSGDQGASGPAG PSGPRGPPGP VGPSGKDGSN
GIPGPIGPPG PRGRSGETGP VGPPGSPGPPGPPGPPGPGI
DMSAFAGLGQ REKGPDPMQY MRADEADSTL RQHDVEVDAT
LKSLNNQIES IRSPDGSRKN PARTCQDLKL CHPEWKSGDY
WIDPNQGCTL DAMKVFCNME TGETCVYPNPATVPRKNWWS
SKSKEKKHIW FGETMNGGFH FSYGDGNLAP NTANVQMTFL
RLLSTEGSQNITYHCKNSIA YLDEAAGNLK KALLIQGSND
VEMRAEGNSR FTYTALKDGC TKHTGKWGKTVIEYRSQKTS
RLPIIDIAPM DIGGAEQEFG VDIGPVCFL.

As used herein, “DERP1” refers to all isoforms, variants, and fragments thereof of a protein associated with the name “Dermatophagoides pteronyssius p1” and known to cause an allergic reaction in humans, or a biological equivalent thereof. A non-limiting exemplary consensus sequence of DERP1 is disclosed herein below:

NEIAXAKIDLRQMRTVTPIXMQGGCGSCWALSGVAATESAYLAYGNXSLD
LAEQELVDCASQHGCHGDTIPRGIEYIQHNGVVQESYYRYVAREQSCRRP
NAQRFGISNYCQIYPPNVNKIREALAQTHSAIAVIIGIKDLDAFRHYDGR
TIIQRDNGYQPNYHAVNIVGYSNAQGVDYWIVRNSWDTNWGDNGYGYFAA
NIDLMMIEEYPYVVIL.

As used herein, “DERP2” refers to all isoforms, variants, and fragments thereof of a protein associated with the name “Dermatophagoides pteronyssius p2” and known to cause an allergic reaction in humans, or a biological equivalent thereof. A non-limiting exemplary consensus sequence of DERP2 is disclosed herein below:

MMYKILCLSLLVAAVARDQVDVKDCANHEIKKVLVPGCHGSEPCIIHRGK
PFQLEAVFEANQNTKTAKIEIKASIDGLEVDVPGIDPNACHYMKCPLVKG
QQYDIKYTWNVPKIAPKSENVVVTVKVMGDDGVLACAIATHAKIRD.

As used herein, “OVA” refers to all isoforms, variants, and fragments thereof of a protein associated with the name “ovalbumin” for use in generating allergic response in mice, or a biological equivalent thereof.

As used herein “BacInt” or “bacteroides integrase” refers to all isoforms, variants, and fragments thereof of a protein associated with that name, or a biological equivalent thereof. The canonical sequence of BacInt is 406 amino acids in length and disclosed herein below:

MDKIRYRLVYNRQNTLNRQGTALVQVEAYLNQRKIYLKTNVYLKPECWSR
EGAQVINHPQSNELNIMLYEYILYLQGIELGYWKRGIPATLSLLKDAVKK
KSAVNISFSTFAKSAIDNSDKKQSTKDNLHSTLAVLHDFRSGLDFKDLTY
TFLRDFEQYLREKGNAVNTIAKHMRQLRTLVNEAINQGYMHADAYPFRKY
KIKQEKGRHEFLTPDELKKLETVEVEEESMRHVLDAFLFCCYTGLRYSDF
CQLTPENFIRINGKRWLYFKSVKTGVEIRLPLHLLFESRALGILDRYPDI
GSFAALPCNSEVNKQLRKLAGLCGIKKRITYHVSRHTCATLLIHQGVAIT
TVQKLLGHTSVKTTQIYSEVLSSTIVRDLKNVQKGKRKVKMFPDKGLRTS
DFIDNR.

As used herein, “CBir,” “Fla-X,” and/or “Fla-2” refers to all isoforms, variants, and fragments thereof of a protein associated with of one or more bacterial flagellins implicated in colitis, or a biological equivalent thereof. A non-limiting exemplary sequence of Fla-X is disclosed herein below:

MVVQHNLRAMNSNRMLGITQGSLNKSTEKLSSGYKVNRAADDAAGLSISE
KMRKQIRGLSQASLNAEDGISAVQTAEGALTEVHDMLQRMNELAVKAANG
TNSTSDRQTIQDEVDQLLTEIDRVAETTKFNELYTLKGDEDKVTRYLSAH
DAGIEGTLTQGATNATFSMDQLKFGDTIMIAGREYHISGTKAEQAAIITA
SVKIGQQVTIDGIMYTCSSVSNADKFELKSEDLIAKLDTSSLSIMSVNGK
TYYGAGITDDRTVVSSIGAYKLIQKELGLASSIGADGATQASVNAGVDGK
TLMKPSFEGKWVFSIDKGSVQVREDIDFSLHVGADADMNNKIAVKIGALD
TKGLGIQGLNVKDTTGAAATYAIDSIADAVARISAQRSLLGAVQNRLEHT
INNLDNVVENTTAAESQIRDTDMATEMVKYSNNNVLAQAGQSMLAQSNQA
NQGVLQLLQ.

A non-limiting exemplary sequence of Fla-2 is disclosed herein below:

MVVQHNLRAMNSNRMLGITQGSLNKSTEKLSSGYKVNRAADDAAGLSISE
KMRKQIRGLSQASLNAEDGISAVQTAEGALTEVHDMLQRMNELAVKAANG
TNSTSDRQTIQDEVDQLLTEIDRVAETTKFNELYTLKGDEDKVTRYLSAH
DAGIEGTLTQGATNATFSMDQLKFGDTIMIAGREYHISGTQKQQGEIITS
SVKIGQQVTIDGIMYTCTATVSNADKFELTKDDLIAKLDTSSLSIMSVNG
KTYYGAGITDDRTVVSSIGAYKLIQKELGLASSIGADGSTQASVNAGVDG
KTLKKPSFEGKWVFSIDKGSVQVREDIDFSLHVGADADMNNKIAVKIGAL
DTKGLGIQGLNVKDTTGAAATYAIDSIADAVARISAQRSLLGAVQNRLEH
TINNLDNVVENTTAAESQIRDTDMATEMVKYSNNNVLAQAGQSMLAQSNQ
ANQGVLSLLG.

As used herein, “YIDX” refers to all isoforms, variants, and fragments thereof of a protein associated with that name, is of bacterial origin, and is implicated in immune related disease pathogenesis, or a biological equivalent thereof. A non-limiting exemplary sequence of YIDX is disclosed herein below:

MKLNFKGFFKAAGLFPLALMLSGCISYALVSHTAKGSSGKYQSQSDTITG
LSQAKDSNGTKGYVFVGESLDYLITDGADDIVKMLNDPALNRHNIQVADD
ARFVLNAGKKKFTGTISLYYYWNNEEEKALATHYGFACGVQHCTRSLENL
KGTIHEKNKNMDYSKVMAFYHPFKVRFYEYYSPRGIPDGVSAALLPVTVT
LDIITAPLQFLVVYAVNQ.

Another non-limiting exemplary sequence of YIDX is disclosed herein below:

MKLNFKGFFKAAGLFPLALMLSGCISYALVSHTAKGSSGKYQSQSDTITG
LSQAKDSNGTKGYVFVGESLDYLITDGADDIVKMLNDPALNRHNIQVADD
ARFVLNAGKKKFTGTISLYYYWNNEEEKALATHYGFACGVQHCTRSLENL
KGTIHEKNKNMDYSKVMAFYHPFKVRFYEYYSPRGIPDGVSAALLPVTVT
LDIITAPLQFLVVYAVNQ.

As used herein, “AChR” refers to all isoforms, variants, and fragments thereof of a protein associated with that name, or a biological equivalent thereof.

A non-limiting exemplary sequence of acetylcholine receptor associated with UniProt Reference No. Q13702-1 is disclosed herein below:

MGQDQTKQQIEKGLQLYQSNQTEKALQVWTKVLEKSSDLMGRFRVLGCLV
TAHSEMGRYKEMLKFAVVQIDTARELEDADFLLESYLNLARSNEKLCEFH
KTISYCKTCLGLPGTRAGAQLGGQVSLSMGNAFLGLSVFQKALESFEKAL
RYAHNNDDAMLECRVCCSLGSFYAQVKDYEKALFFPCKAAELVNNYGKGW
SLKYRAMSQYHMAVAYRLLGRLGSAMECCEESMKIALQHGDRPLQALCLL
CFADIHRSRGDLETAFPRYDSAMSIMTEIGNRLGQVQALLGVAKCWVARK
ALDKALDAIERAQDLAEEVGNKLSQLKLHCLSESIYRSKGLQRELRAHVV
RFHECVEETELYCGLCGESIGEKNSRLQALPCSHIFHLRCLQNNGTRSCP
NCRRSSMKPGFV.

A non-limiting exemplary sequence of acetylcholine receptor associated with UniProt Reference No. Q04844-1 is disclosed herein below:

MARAPLGVLLLLGLLGRGVGKNEELRLYHHLFNNYDPGSRPVREPEDTVT
ISLKVTLTNLISLNEKEETLTTSVWIGIDWQDYRLNYSKDDFGGIETLRV
PSELVWLPEIVLENNIDGQFGVAYDANVLVYEGGSVTWLPPAIYRSVCAV
EVTYFPFDWQNCSLIFRSQTYNAEEVEFTFAVDNDGKTINKIDIDTEAYT
ENGEWAIDFCPGVIRRHHGGATDGPGETDVIYSLIIRRKPLFYVINIIVP
CVLISGLVLLAYFLPAQAGGQKCTVSINVLLAQTVFLFLIAQKIPETSLS
VPLLGRFLIFVMVVATLIVMNCVIVLNVSQRTPTTHAMSPRLRHVLLELL
PRLLGSPPPPEAPRAASPPRRASSVGLLLRAEELILKKPRSELVFEGQRH
RQGTWTAAFCQSLGAAAPEVRCCVDAVNFVAESTRDQEATGEEVSDWVRM
GNALDNICFWAALVLFSVGSSLIFLGAYFNRVPDLPYAPCIQP.

A non-limiting exemplary sequence of acetylcholine receptor associated with UniProt Reference No. P02708-1 is disclosed herein below:

MEPWPLLLLFSLCSAGLVLGSEHETRLVAKLFKDYSSVVRPVEDHRQVVE
VTVGLQLIQLINVDEVNQIVTTNVRLKQGDMVDLPRPSCVTLGVPLFSHL
QNEQWVDYNLKWNPDDYGGVKKIHIPSEKIWRPDLVLYNNADGDFAIVKF
TKVLLQYTGHITWTPPAIFKSYCEIIVTHFPFDEQNCSMKLGTWTYDGSV
VAINPESDQPDLSNFMESGEWVIKESRGWKHSVTYSCCPDTPYLDITYHF
VMQRLPLYFIVNVIIPCLLFSFLTGLVFYLPTDSGEKMTLSISVLLSLTV
FLLVIVELIPSTSSAVPLIGKYMLFTMVFVIASIIITVIVINTHHRSPST
HVMPNWVRKVFIDTIPNIMFFSTMKRPSREKQDKKIFTEDIDISDISGKP
GPPPMGFHSPLIKHPEVKSAIEGIKYIAETMKSDQESNNAAAEWKYVAMV
MDHILLGVFMLVCIIGTLAVFAGRLIELNQQG.

A non-limiting exemplary sequence of acetylcholine receptor associated with UniProt Reference No. P07510-1 is disclosed herein below:

MHGGQGPLLLLLLLAVCLGAQGRNQEERLLADLMQNYDPNLRPAERDSDV
VNVSLKLTLTNLISLNEREEALTTNVWIEMQWCDYRLRWDPRDYEGLWVL
RVPSTMVWRPDIVLENNVDGVFEVALYCNVLVSPDGCIYWLPPAIFRSAC
SISVTYFPFDWQNCSLIFQSQTYSTNEIDLQLSQEDGQTIEWIFIDPEAF
TENGEWAIQHRPAKMLLDPAAPAQEAGHQKVVFYLLIQRKPLFYVINIIA
PCVLISSVAILIHFLPAKAGGQKCTVAINVLLAQTVFLFLVAKKVPETSQ
AVPLISKYLTFLLVVTILIVVNAVVVLNVSLRSPHTHSMARGVRKVFLRL
LPQLLRMHVRPLAPAAVQDTQSRLQNGSSGWSITTGEEVALCLPRSELLF
QQWQRQGLVAAALEKLEKGPELGLSQFCGSLKQAAPAIQACVEACNLIAC
ARHQQSHFDNGNEEWFLVGRVLDRVCFLAMLSLFICGTAGIFLMAHYNRV
PALPFPGDPRPYLPSPD.

A non-limiting exemplary sequence of acetylcholine receptor associated with UniProt Reference No. P11230-1 is disclosed herein below:

MTPGALLMLLGALGAPLAPGVRGSEAEGRLREKLFSGYDSSVRPAREVGD
RVRVSVGLILAQLISLNEKDEEMSTKVYLDLEWTDYRLSWDPAEHDGIDS
LRITAESVWLPDVVLLNNNDGNFDVALDISVVVSSDGSVRWQPPGIYRSS
CSIQVTYFPFDWQNCTMVFSSYSYDSSEVSLQTGLGPDGQGHQEIHIHEG
TFIENGQWEIIHKPSRLIQPPGDPRGGREGQRQEVIFYLIIRRKPLFYLV
NVIAPCILITLLAIFVFYLPPDAGEKMGLSIFALLTLTVFLLLLADKVPE
TSLSVPIIIKYLMFTMVLVTFSVILSVVVLNLHHRSPHTHQMPLWVRQIF
IHKLPLYLRLKRPKPERDLMPEPPHCSSPGSGWGRGTDEYFIRKPPSDFL
FPKPNRFQPELSAPDLRRFIDGPNRAVALLPELREVVSSISYIARQLQEQ
EDHDALKEDWQFVAMVVDRLFLWTFIIFTSVGTLVIFLDATYHLPPPDPF
P

A non-limiting exemplary sequence of acetylcholine receptor associated with UniProt Reference No. Q07001-1 is disclosed herein below:

MEGPVLTLGLLAALAVCGSWGLNEEERLIRHLFQEKGYNKELRPVAHKEE
SVDVALALTLSNLISLKEVEETLTTNVWIEHGWTDNRLKWNAEEFGNISV
LRLPPDMVWLPEIVLENNNDGSFQISYSCNVLVYHYGFVYWLPPAIFRSS
CPISVTYFPFDWQNCSLKFSSLKYTAKEITLSLKQDAKENRTYPVEWIII
DPEGFTENGEWEIVHRPARVNVDPRAPLDSPSRQDITFYLIIRRKPLFYI
INILVPCVLISFMVNLVFYLPADSGEKTSVAISVLLAQSVFLLLISKRLP
ATSMAIPLIGKFLLFGMVLVTMVVVICVIVLNIHFRTPSTHVLSEGVKKL
FLETLPELLHMSRPAEDGPSPGALVRRSSSLGYISKAEEYFLLKSRSDLM
FEKQSERHGLARRLTTARRPPASSEQAQQELFNELKPAVDGANFIVNHMR
DQNNYNEEKDSWNRVARTVDRLCLFVVTPVMVVGTAWIFLQGVYNQPPPQ
PFPGDPYSYNVQDKRFI.

As used herein, “thyroid peroxidase” refers to all isoforms, variants, and fragments thereof of a protein associated with that name, or a biological equivalent thereof. A non-limiting exemplary sequence of human thyroid peroxidase associated with UniProt Reference

MRALAVLSVTLVMACTEAFFPFISRGKELLWGKPEESRVSSVLEESKRLV
DTAMYATMQRNLKKRGILSPAQLLSFSKLPEPTSGVIARAAEIMETSIQA
MKRKVNLKTQQSQHPTDALSEDLLSIIANMSGCLPYMLPPKCPNTCLANK
YRPITGACNNRDHPRWGASNTALARWLPPVYEDGFSQPRGWNPGFLYNGF
PLPPVREVTRHVIQVSNEVVTDDDRYSDLLMAWGQYIDHDIAFTPQSTSK
AAFGGGADCQMTCENQNPCFPIQLPEEARPAAGTACLPFYRSSAACGTGD
QGALFGNLSTANPRQQMNGLTSFLDASTVYGSSPALERQLRNWTSAEGLL
RVHARLRDSGRAYLPFVPPRAPAACAPEPGIPGETRGPCFLAGDGRASEV
PSLTALHTLWLREHNRLAAALKALNAHWSADAVYQEARKVVGALHQIITL
RDYIPRILGPEAFQQYVGPYEGYDSTANPTVSNVFSTAAFRFGHATIHPL
VRRLDASFQEHPDLPGLWLHQAFFSPWTLLRGGGLDPLIRGLLARPAKLQ
VQDQLMNEELTERLFVLSNSSTLDLASINLQRGRDHGLPGYNEWREFCGL
PRLETPADLSTAIASRSVADKILDLYKHPDNIDVWLGGLAENFLPRARTG
PLFACLIGKQMKALRDGDWFWWENSHVFTDAQRRELEKHSLSRVICDNTG
LTRVPMDAFQVGKFPEDFESCDSITGMNLEAWRETFPQDDKCGFPESVEN
GDFVHCEESGRRVLVYSCRHGYELQGREQLTCTQEGWDFQPPLCKDVNEC
ADGAHPPCHASARCRNTKGGFQCLCADPYELGDDGRTCVDSGRLPRVTWI
SMSLAALLIGGFAGLTSTVICRWTRTGTKSTLPISETGGGTPELRCGKHQ
AVGTSPQRAAAQDSEQESAGMEGRDTHRLPRAL.

As used herein, “thyroid receptor” refers to all isoforms, variants, and fragments thereof of a protein associated with that name, or a biological equivalent thereof. In some embodiments, “thyroid receptor” includes “thyroid stimulating hormone receptor.” A non-limiting exemplary sequence of thyroid stimulating hormone receptor associated with UniProt Reference No. P16473-1 is disclosed herein below:

MRPADLLQLVLLLDLPRDLGGMGCSSPPCECHQEEDFRVTCKDIQRIPSL
PPSTQTLKLIETHLRTIPSHAFSNLPNISRIYVSIDVTLQQLESHSFYNL
SKVTHIEIRNTRNLTYIDPDALKELPLLKFLGIFNTGLKMFPDLTKVYST
DIFFILEITDNPYMTSIPVNAFQGLCNETLTLKLYNNGFTSVQGYAFNGT
KLDAVYLNKNKYLTVIDKDAFGGVYSGPSLLDVSQTSVTALPSKGLEHLK
ELIARNTWTLKKLPLSLSFLHLTRADLSYPSHCCAFKNQKKIRGILESLM
CNESSMQSLRQRKSVNALNSPLHQEYEENLGDSIVGYKEKSKFQDTHNNA
HYYVFFEEQEDEIIGFGQELKNPQEETLQAFDSHYDYTICGDSEDMVCTP
KSDEFNPCEDIMGYKFLRIVVWFVSLLALLGNVFVLLILLTSHYKLNVPR
FLMCNLAFADFCMGMYLLLIASVDLYTHSEYYNHAIDWQTGPGCNTAGFF
TVFASELSVYTLTVITLERWYAITFAMRLDRKIRLRHACAIMVGGWVCCF
LLALLPLVGISSYAKVSICLPMDTETPLALAYIVFVLTLNIVAFVIVCCC
YVKIYITVRNPQYNPGDKDTKIAKRMAVLIFTDFICMAPISFYALSAILN
KPLITVSNSKILLVLFYPLNSCANPFLYAIFTKAFQRDVFILLSKFGICK
RQAQAYRGQRVPPKNSTDIQVQKVTHDMRQGLHNMEDVYELIENSHLTPK
KQGQISEEYMQTVL.

A non-limiting exemplary sequence of thyroid stimulating hormone receptor associated with UniProt Reference No. Q59GA2-1 is disclosed herein below:

PRVPWKMRPADLLQLVLLLDLPRDLGGMGCSSPPCECHQEEDFRVTCKDI
QRIPSLPPSTQTLKLIETHLRTIPSHAFSNLPNISRIYVSIDVTLQQLES
HSFYNLSKVTHIEIRNTRNLTYIDPDALKELPLLKFLGIFNTGLKMFPDL
TKVYSTDIFFILEITDNPYMTSIPVNAFQGLCNETLTLKLYNNGFTSVQG
YAFNGTKLDAVYLNKNKYLTVIDKDAFGGVYSGPSLLDVSQTSVTALPSK
GLEHLKELIARNTWTLKKLPLSLSFLHLTRADLSYPSHCCAFKNQKKIRG
ILESLMCNESSMQSLRQRKSVNALNSPLHQEYEENLGDSIVGYKEKSKFQ
DTHNNAHYYVFFEEQEDEIIGFGQELKNPQEETLQAFDSHYDYTICGDSE
DMVCTPKSDEFNPCEDIMGYKFLRIVVWFVSLLALLGNVFVLLILLTSHY
KLNVPRFLMCNLAFADFCMGMYLLLIASVDLYTHSEYYNHAIDWQTGPGC
NTAGFFTVFASELSVYTLTVITLERWYAITFAMRLDRKIRLRHACAIMVG
GWVCCFLLALLPLVGISSYAKVSICLPMDTETPLALAYIVFVLTLNIVAF
VIVCCCYVKIYITVRNPQYNPGDKDTKIAKRMAVLIFTDFICMAPISFYA
LSAILNKPLITVSNSKILLVLFYPLNSCANPFLYAIFTKAFQRDVFILLS
KFGICKRQAQAYRGQRVPPKNSTDIQVQKVTHDMRQGLHNMEDVYELIEN
SHLTPKKQGQISEEYMQTVL.

A non-limiting exemplary sequence of thyroid stimulating hormone receptor associated with UniProt Reference No. B4E0H2-1 is disclosed herein below:

MRPADLLQLVLLLDLPRDLGGMGCSSPPCECHQEEDFRVTCKDIQRIPSL
PPSTQTLKLIETHLRIVVWFVSLLALLGNVFVLLILLTSHYKLNVPRFLM
CNLAFADFCMGMYLLLIASVDLYTHSEYYNHAIDWQTGPGCNTAGFFTVF
ASELSVYTLTVITLERWYAITFAMRLDRKIRLRHACAIMVGGWVCCFLLA
LLPLVGISSYAKVSICLPMDTETPLALAYIVFVLTLNIVAFVIVCCCYVK
IYITVRNPQYNPGDKDTKIAKRMAVLIFTDFICMAPISFYALSAILNKPL
ITVSNSKILLVLFYPLNSCANPFLYAIFTKAFQRDVFILLSKFGICKRQA
QAYRGQRVPPKNSTDIQVQKVTHEMRQGLHNMEDVYELIENSHLTPKKQG
QISEEYMQTVL.

As used herein, “phospholipid antigen” refers to all isoforms, variants, and fragments thereof of a protein associated with that name, or a biological equivalent thereof. One non-limiting example of a phospholipid antigen is “beta2-glycoprotein I”, whose sequence is disclosed herein below:

MISPVLILFSSFLCHVAIAGRTCPKPDDLPFSTVVPLKTFYEPGEEITYS
CKPGYVSRGGMRKFICPLTGLWPINTLKCTPRVCPFAGILENGAVRYTTF
EYPNTISFSCNTGFYLNGADSAKCTEEGKWSPELPVCAPIICPPPSIPTF
ATLRVYKPSAGNNSLYRDTAVFECLPQHAMFGNDTITCTTHGNWTKLPEC
REVKCPFPSRPDNGFVNYPAKPTLYYKDKATFGCHDGYSLDGPEEIECTK
LGNWSAMPSCKASCKVPVKKATVVYQGERVKIQEKFKNGMLHGDKVSFFC
KNKEKKCSYTEDAQCIDGTIEVPKCFKEHSSLAFWKTDASDVKPC

As used herein, “H4” refers to all isoforms, variants, and fragments thereof of a protein associated with the name “histone H4”, or a biological equivalent thereof. The canonical sequence H4 is disclosed herein below:

SGRGKGGKGLGKGGAKRHRKVLRDNIQGITKPAIRRLARRGGVKRISGLI
YEETRGVLKVFLENVIRDAVTYTEHAKRKTVTAMDVVYALKRQGRTLYGF
GG.

As used herein, “H2B” refers to all isoforms, variants, and fragments thereof of a protein associated with the name “histone H2B”, or a biological equivalent thereof. The canonical sequence of H2B is disclosed herein below:

PEPAKSAPAPKKGSKKAVTKAQKKDGKKRKRSRKESYSVYVYKVLKQVHP
DTGISSKAMGIMNSFVNDIFERIASEASRLAHYNKRSTITSREIQTAVRL
LLPGELAKHAVSEGTKAVTKYTSSK.

As used herein, “H1” refers to all isoforms, variants, and fragments thereof of a protein associated with the name “histone H1”, or a biological equivalent thereof. The canonical sequence of H1 is disclosed herein below:

MTENSTSAPAAKPKRAKASKKSTDHPKYSDMIVAAIQAEKNRAGSSRQSI
QKYIKSHYKVGENADSQIKLSIKRLVTTGVLKQTKGVGASGSFRLAKGDE
PKRSVAFKKTKKEVKKVATPKKAAKPKKAASKAPSKKPKATPVKKAKKKP
AATPKKAKKPKVVKVKPVKASKPKKAKTVKPKAKSSAKRASKKK.

As used herein, “ApoB” refers to all isoforms, variants, and fragments thereof of a protein associated with the name “apolipoprotein B”, or a biological equivalent thereof. The canonical sequence of ApoB is disclosed herein below:

MGPRKPALRTPLLLLFLLLFLDTSVWAQDEVLENLSFSCPKDATRFKHLRKYVYNYE
AESSSGVQGTADSRSATKINCKVELEVPQICGFIMRTNQCTLKEVYGFNPEGKALMK
KTKNSEEFAAAMSRYELKLAIPEGKQIVLYPDKDEPKYILNIKRGIISALLVPPETEED
QQELFLDTVYGNCSTQVTVNSRKGTVPTEMSTERNLQQCDGFQPISTSVSPLALIKGL
VHPLSTLISSSQTCQYTLDPKRKHVSEAVCDEQHLFLPFSYKNKYGIMTRVTQKLSLE
DTPKINSRFFSEGTNRMGLAFESTKSTSSPKQADAVLKTLQELKKLSISEQNAQRANL
FNKLVTELRGLTGEAITSLLPQLIEVSSPITLQALVQCGQPQCYTHILQWLKTEKAHPL
LVDIVTYLMALIPNPSTQRLQEIFNTAKEQQSRATLYALSHAVNSYFDVDHSRSPVLQ
DIAGYLLKQIDNECTGNEDHTFLILRVIGNMGRTMEQVMPALKSSVLSCVRSTKPSLL
IQKAALQALRKMELEDEVRTILFDTFVNGVAPVEKRLAAYLLLMKNPSSSDINKIAQ
LLQWEQSEQVKNFVASHIANILNSEELYVQDLKVLIKNALENSQFPTIMDFRKFSRNY
QISKSASLPMFDPVSVKIEGNLIFDPSSYLPRESLLKTTLTVFGLASLDLFEIGLEGKGF
EPTLEALFGKQGFFPDSVNKALYWVNGRVPDGVSKVLVDHFGYTTDGKHEQDMVN
GIMPIVDKLIKDLKSKEIPEARAYLRILGKELSFVRLQDLQVLGKLLLSGAQTLQGIPQ
MVVQAIREGSKNDLFLHYIFMDNAFELPTGAGLQLQVSSSGVFTPGIKAGVRLELANI
QAELVAKPSVSLEFVTNMGIIIPDFAKSSVQMNTNFFHESGLEARVALKAGQLKVIIPS
PKRPVKLFSGSNTLHLVSTTKTEVIPPLVENRQSWSTCKPLFTGMNYCTTGAYSNASS
TESASYYPLTGDTRYELELRPTGEVEQYSATATYELLKEDKSLVDTLKFLVQAEGVQ
QSEATVLFKYNRRSRTLSSEVLIPGFDVNFGTILRVNDESAKDKNTYKLILDIQNKKIT
EVSLVGHLSYDKKGDGKIKGVVSIPRLQAEARSEVHTHWSSTKLLFQMDSSATAYG
STISKRVTWRYDNEIIEFDWNTGTNVDTKKVASNFPVDLSHYPRMLHEYANGLLDH
RVPQTDVTFRDMGSKLIVATNTWLQMATRGLPYPQTLQDHLNSLSELNLLKMGLSD
FHIPDNLFLKTDGRVKYTMNRNKINIDIPLPLGGKSSKDLKMPESVRTPALNFKSVGF
HLPSREVQVPTFTIPKTHQLQVPLLGVLDLSTNVYSNLYNWSASYTGGNTSRDHFSL
QAQYRMKTDSVVDLFSYSVQGSGETTYDSKNTFTLSCDGSLHHKFLDSKFKVSHVE
KFGNSPVSKGLLTFETSSALGPQMSATVHLDSKKKQHLYVKDIKVDGQFRASSFYAQ
GKYGLSCERDVTTGQLSGESNMRFNSTYFQGTNQIVGMYQDGALSITSTSDLQDGIF
KNTASLKYENYELTLKSDSSGQYENFAASNKLDVTFSTQSALLRSEHQANYKSLRLV
TLLSGSLTSQGVELNADILGTDKINTGAHKATLKIARDGLSTSATTNLKYSPLLLENE
LNAELGLSGASMKLSTNGRFKEHHAKFSLDGRAALTEVSLGSIYQAMILGADSKNIF
NFKLSREGLRLSNDLMGSYAEMKLDHTHSLNIAGLSLDFFSKMDNIYSGDKFYKQNF
NLQLQPYSFITTLSNDLRYGALDLTNNGRFRLEPLKLNVGGNFKGTYQNNELKHIYTI
SYTDLVVASYRADTVAKVQGVEFSHRLNADIEGLTSSVDVTTSYNSDPLHFNNVFHF
SLAPFTLGIDTHTSGDGKLSFWGEHTGQLYSKFLLKAEPLALIVSHDYKGSTSHSLPY
ESSISTALEHTVSALLTPAEQTSTWKFKTKLNDKVYSQDFEAYNTKDKIGVELSGRA
DLSGLYSPIKLPFFYSEPVNVLNGLEVNDAVDKPQEFTIIAVVKYDKNQDVHTINLPF
FKSLPDYLERNRRGMISLLEAMRGELQRLSVDQFVRKYRAALSRLPQQIHHYLNASD
WERQVAGAKEKITSFMENYRITDNDVLIAIDSAKINFNEKLSQLETYAIQFDQYIKDN
YDPHDLKRTIAEIIDRIIEKLKILDEQYHIRVNLAKSIHNLYLFVENVDLNQVSSSNTS
WIQNVDSNYQVRIQIQEKLQQLRTQIQNIDIQQLAAEVKRQMDAIDVTMHLDQLRTA
ILFQRISDIIDRVKYFVMNLIEDFKVTEKINTFRVIVRELIEKYEVDQHIQVLMDKSVEL
AHRYSLSEPLQKLSNVLQRIEIKDYYEKLVGFIDDTVEWLKALSFKNTIEELNRLTDM
LVKKLKAFDYHQFVDKTNSKIREMTQRINAEIQALKLPQKMEALKLLVEDFKTTVSN
SLERLKDTKVTVVIDWLQDILTQMKDHFQDTLEDVRDRIYQMDIQRELEHFLSLVNQ
VYSTLVTYMSDWWTLTAKNITDFAEQYSIQNWAESIKVLVEQGFIVPEMQTFLWTM
PAFEVSLRALQEGNFQTPVFIVPLTDLRIPSIRINFKMLKNIKIPLRFSTPEFTLLNTFHV
HSFTIDLLEIKAKIIRTIDQILSSELQWPLPEMYLRDLDVVNIPLARLTLPDFHVPEITIPE
FTIPNVNLKDLHVPDLHIPEFQLPHLSHTIEIPAFGKLHSILKIQSPLFILDANANIQNVT
TSGNKAEIVASVTAKGESQFEALNFDFQAQAQFLELNPHPPVLKESMNFSSKHVRME
HEGEIVFDGKAIEGKSDTVASLHTEKNEVEFNNGMTVKVNNQLTLDSHTKYFHKLS
VPRLDFSSKASLNNEIKTLLEAGHVALTSSGTGSWNWACPNFSDEGIHSSQISFTVDG
PIAFVGLSNNINGKHLRVIQKLTYESGFLNYSKFEVESKVESQHVGSSILTANGRALL
KDAKAEMTGEHNANLNGKVIGTLKNSLFFSAQPFEITASTNNEGNLKVGFPLKLTGK
IDFLNNYALFLSPRAQQASWQASTRFNQYKYNQNFSAINNEHNIEASIGMNGDANLD
FLNIPLTIPEINLPYTEFKTPLLKDFSIWEETGLKEFLKTTKQSFDLSVKAQYKKNSDK
HSIVVPLGMFYEFILNNVNSWDRKFEKVRNNALHFLTTSYNEAKIKVDKYKTENSLN
QPSGTFQNHGYTIPVVNIEVSPFAVETLASSHVIPTAISTPSVTIPGPNIMVPSYKLVLPP
LELPVFHGPGNLFKFFLPDFKGFNTIDNIYIPAMGNFTYDFSFKSSVITLNTNAGLYNQ
SDIVAHFLSSSSFVTDALQYKLEGTSRLMRKRGLKLATAVSLTNKFVKGSHDSTISLT
KKNMEASVRTTANLHAPIFSMNFKQELNGNTKSKPTVSSSIELNYDFNSSKLHSTAT
GGIDHKFSLESLTSYFSIESFTKGNIKSSFLSQEYSGSVANEANVYLNSKGTRSSVRLQ
GASKVDGIWNVEVGENFAGEATLQRIYTTWEHNMKNHLQVYSYFFTKGKQTCRAT
LELSPWTMSTLLQVHVSQLSSLLDLHHFDQEVILKANTKNQKISWKGGVQVESRVL
QHNAQFSNDQEEIRLDLAGSLDGQLWDLEAIFLPVYGKSLQELLQMDGKRQYLQAS
TSLLYTKNPNGYLLSLPVQELADRFIIPGIKLNDFSGVKIYKKLSTSPFALNLTMLPKV
KFPGIDLLTQYSTPEGSSVPIFEATIPEIHLTVSQFTLPKSLPVGNTVFDLNKLANMIAD
VDLPSVTLPEQTIVIPPLEFSVPAGIFIPFFGELTARAGMASPLYNVTWSAGWKTKAD
HVETFLDSMCTSTLQFLEYALKVVETHKIEEDLLTYNIKGTLQHCDFNVEYNEDGLF
KGLWDWQGEAHLDITSPALTDFHLYYKEDKTSLSASAASSTIGTVGLDSSTDDQSVE
LNVYFHPQSPPEKKLSIFKTEWRYKESDGERYIKINWEEEAASRLLGSLKSNVPKASK
AIYDYANKYHLEYVSSELRKSLQVNAEHARRMVDEMNMSFQRVARDTYQNLYEE
MLAQKSLSIPENLKKRVLDSIVHVTQKYHMAVMWLMDSFIHFLKFNRVQFPGYAGT
YTVDELYTIVMKETKKSLSQLFNGLGNLLSYVQNQVEKSRLINDITFKCPFFSKPCKL
KDLILIFREELNILSNIGQQDIKFTTILSSLQGFLERVLDIIEEQIKCLKDNESTCVADHIN
MVFKIQVPYAFKSLREDIYFVLGEFNDFLQSILQEGSYKLQQVHQYMKALREEYFDP
SMVGWTVKYYEIEENMVELIKTLLVSFRDVYSEYSVTAADFASKMSTQVEQFVSRDI
REYLSMLTDINGKWMEKIAELSIVAKETMKSWVTAVAKIMSDYPQQFHSNLQDFSD
QLSSYYEKFVGESTRLIDLSIQNYHVFLRYITELLRKLQVATANNVSPYIKLAQGELMI
TF.

As used herein, “ApoE” refers to all isoforms, variants, and fragments thereof of a protein associated with the name “apolipoprotein E”, or a biological equivalent thereof. A non-limiting exemplary sequence of human apoE associated with UniProt Reference No. P02649 is disclosed herein below:

MKVLWAALLVTFLAGCQAKVEQAVETEPEPELRQQTEWQSGQRWELALGR
FWDYLRWVQTLSEQVQEELLSSQVTQELRALMDETMKELKAYKSELEEQL
TPVAEETRARLSKELQAAQARLGADMEDVCGRLVQYRGEVQAMLGQSTEE
LRVRLASHLRKLRKRLLRDADDLQKRLAVYQAGAREGAERGLSAIRERLG
PLVEQGRVRAATVGSLAGQPLQERAQAWGERLRARMEEMGSRTRDRLDEV
KEQVAEVRAKLEEQAQQIRLQAEAFQARLKSWFEPLVEDMQRQWAGLVEK
VQAAVGTSAAPVPSDNH.

As used herein, “NMDAR” refers to all isoforms, variants, and fragments thereof of a protein associated with the name “N-methyl-D-aspartate receptor”, or a biological equivalent thereof. A non-limiting exemplary sequence of N-methyl-D-asparate receptor associated with UniProt Reference No. Q13224-1 is disclosed herein below:

MKPRAECCSPKFWLVLAVLAVSGSRARSQKSPPSIGIAVILVGTSDEVAI
KDAHEKDDFHHLSVVPRVELVAMNETDPKSIITRICDLMSDRKIQGVVFA
DDTDQEAIAQILDFISAQTLTPILGIHGGSSMIMADKDESSMFFQFGPSI
EQQASVMLNIMEEYDWYIFSIVTTYFPGYQDFVNKIRSTIENSFVGWELE
EVLLLDMSLDDGDSKIQNQLKKLQSPIILLYCTKEEATYIFEVANSVGLT
GYGYTWIVPSLVAGDTDTVPAEFPTGLISVSYDEWDYGLPARVRDGIAII
TTAASDMLSEHSFIPEPKSSCYNTHEKRIYQSNMLNRYLINVTFEGRNLS
FSEDGYQMHPKLVIILLNKERKWERVGKWKDKSLQMKYYVWPRMCPETEE
QEDDHLSIVTLEEAPFVIVESVDPLSGTCMRNTVPCQKRIVTENKTDEEP
GYIKKCCKGFCIDILKKISKSVKFTYDLYLVTNGKHGKKINGTWNGMIGE
VVMKRAYMAVGSLTINEERSEVVDFSVPFIETGISVMVSRSNGTVSPSAF
LEPFSADVWVMMFVMLLIVSAVAVFVFEYFSPVGYNRCLADGREPGGPSF
TIGKAIWLLWGLVFNNSVPVQNPKGTTSKIMVSVWAFFAVIFLASYTANL
AAFMIQEEYVDQVSGLSDKKFQRPNDFSPPFRFGTVPNGSTERNIRNNYA
EMHAYMGKFNQRGVDDALLSLKTGKLDAFIYDAAVLNYMAGRDEGCKLVT
IGSGKVFASTGYGIAIQKDSGWKRQVDLAILQLFGDGEMEELEALWLTGI
CHNEKNEVMSSQLDIDNMAGVFYMLGAAMALSLITFICEHLFYWQFRHCF
MGVCSGKPGMVFSISRGIYSCIHGVAIEERQSVMNSPTATMNNTHSNILR
LLRTAKNMANLSGVNGSPQSALDFIRRESSVYDISEHRRSFTHSDCKSYN
NPPCEENLFSDYISEVERTFGNLQLKDSNVYQDHYHHHHRPHSIGSASSI
DGLYDCDNPPFTTQSRSISKKPLDIGLPSSKHSQLSDLYGKFSFKSDRYS
GHDDLIRSDVSDISTHTVTYGNIEGNAAKRRKQQYKDSLKKRPASAKSRR
EFDEIELAYRRRPPRSPDHKRYFRDKEGLRDFYLDQFRTENSPHWEHVDL
TDIYKERSDDFKRDSVSGGGPCTNRSHIKHGTGDKHGVVSGVPAPWEKNL
TNVEWEDRSGGNFCRSCPSKLHNYSTTVTGQNSGRQACIRCEACKKAGNL
YDISEDNSLQELDQPAAPVAVTSNASTTKYPQSPTNSKAQKKNRNKLRRQ
HSYDTFVDLQKEEALAPRSVSLKDKGRFMDGSPYAHMFEMSAGESTFANN
KSSVPTAGHHHHNNPGGGYLSKSLYPDRVTQNPFIPTFGDDQCLLHGSKS
YFFRQPTVAGASKARPDFRALVTNKPVSALHGAVPARFQKDICIGNQSNP
CVPNNKNPRAFNGSSNGHVYEKLSSIESDV.

As used herein, “voltage-gated potassium channel” refers generally to a transmembrane channel specific for potassium and sensitive to voltage changes in a cell's membrane potential. During action potentials, said channels play a crucial role in returning the depolarized cell to a resting state. A non-limiting exemplary sequence of voltage-gated potassium channel associated with UniProt Reference No. P22459-1 is disclosed herein below:

MEVAMVSAESSGCNSHMPYGYAAQARARERERLAHSRAAAAAAVAAATAA
VEGSGGSGGGSHHHHQSRGACTSHDPQSSRGSRRRRRQRSEKKKAHYRQS
SFPHCSDLMPSGSEEKILRELSEEEEDEEEEEEEEEEGRFYYSEDDHGDE
CSYTDLLPQDEGGGGYSSVRYSDCCERVVINVSGLRFETQMKTLAQFPET
LLGDPEKRTQYFDPLRNEYFFDRNRPSFDAILYYYQSGGRLKRPVNVPFD
IFTEEVKFYQLGEEALLKFREDEGFVREEEDRALPENEFKKQIWLLFEYP
ESSSPARGIAIVSVLVILISIVIFCLETLPEFRDDRDLVMALSAGGHGGL
LNDTSAPHLENSGHTIFNDPFFIVETVCIVWFSFEFVVRCFACPSQALFF
KNIMNIIDIVSILPYFITLGTDLAQQQGGGNGQQQQAMSFAILRIIRLVR
VFRIFKLSRHSKGLQILGHTLRASMRELGLLIFFLFIGVILFSSAVYFAE
ADEPTTHFQSIPDAFWWAVVTMTTVGYGDMKPITVGGKIVGSLCAIAGVL
TIALPVPVIVSNFNYFYHRETENEEQTQLTQNAVSCPYLPSNLLKKFRSS
TSSSLGDKSEYLEMEEGVKESLCAKEEKCQGKGDDSETDKNNCSNAKAVE
TDV.

As used herein, “Elastin” refers to all isoforms, variants, and fragments thereof of a protein associated with that name, or a biological equivalent thereof. The canonical sequence of elastin is 786 amino acids in length and is disclosed herein below:

MAGLTAAAPR PGVLLLLLSI LHPSRPGGVP GAIPGGVPGG
VFYPGAGLGA LGGGALGPGGKPLKPVPGGL AGAGLGAGLG
AFPAVTFPGA LVPGGVADAA AAYKAAKAGA GLGGVPGVGG
LGVSAGAVVP QPGAGVKPGK VPGVGLPGVY PGGVLPGARF
PGVGVLPGVP TGAGVKPKAPGVGGAFAGIP GVGPFGGPQP
GVPLGYPIKA PKLPGGYGLP YTTGKLPYGY GPGGVAGAAG
KAGYPTGTGV GPQAAAAAAA KAAAKFGAGA AGVLPGVGGA
GVPGVPGAIP GIGGIAGVGT PAAAAAAAAA AKAAKYGAAA
GLVPGGPGFG PGVVGVPGAG VPGVGVPGAG IPVVPGAGIP
GAAVPGVVSP EAAAKAAAKA AKYGARPGVG VGGIPTYGVG
AGGFPGFGVG VGGIPGVAGV PGVGGVPGVG GVPGVGISPE
AQAAAAAKAA KYGAAGAGVL GGLVPGPQAA VPGVPGTGGV
PGVGTPAAAA AKAAAKAAQF GLVPGVGVAP GVGVAPGVGV
APGVGLAPGV GVAPGVGVAP GVGVAPGIGP GGVAAAAKSA
AKVAAKAQLR AAAGLGAGIP GLGVGVGVPG LGVGAGVPGL
GVGAGVPGFG AGADEGVRRS LSPELREGDP SSSQHLPSTP
SSPRVPGALA AAKAAKYGAA VPGVLGGLGA LGGVGIPGGV
VGAGPAAAAA AAKAAAKAAQ FGLVGAAGLG GLGVGGLGVP
GVGGLGGIPP AAAAKAAKYG AAGLGGVLGG AGQFPLGGVA
ARPGFGLSPI FPGGACLGKA CGRKRK.

As used herein, “IRBP” refers to all isoforms, variants, and fragments thereof of a protein associated with the name “interphotoreceptor retinoid binding protein”, or a biological equivalent thereof.

As used herein, “arresting human retinal S antigen” refers to all isoforms, variants, and fragments thereof of a protein associated with that name, or a biological equivalent thereof. One non-limiting exemplary sequence is disclosed herein below:

MAASGKTSKSEPNHVIFKKISRDKSVTIYLGNRDYIDHVSQVQPVDGVVL
VDPDLVKGKKVYVTLTCAFRYGQEDIDVIGLTFRRDLYFSRVQVYPPVGA
ASTPTKLQESLLKKLGSNTYPFLLTFPDYLPCSVMLQPAPQDSGKSCGVD
FEVKAFATDSTDAEEDKIPKKSSVRLLIRKVQHAPLEMGPQPRAEAAWQF
FMSDKPLHLAVSLNKEIYFHGEPIPVTVTVTNNTEKTVKKIKAFVEQVAN
VVLYSSDYYVKPVAMEEAQEKVPPNSTLTKTLTLLPLLANNRERRGIALD
GKIKHEDTNLASSTIIKEGIDRTVLGILVSYQIKVKLTVSGFLGELTSSE
VATEVPFRLMHPQPEDPAKESYQDANLVFEEFARHNLKDAGEAEEGKRDK
NDVDE

As used herein, “myosin” refers to all isoforms, variants, and fragments thereof of a protein associated with that name, or a biological equivalent thereof. A non-limiting exemplary sequence of myosin associated with UniProt Reference No. P35580-1 is disclosed herein below:

MAQRTGLEDPERYLFVDRAVIYNPATQADWTAKKLVWIPSERHGFEAASI
KEERGDEVMVELAENGKKAMVNKDDIQKMNPPKFSKVEDMAELTCLNEAS
VLHNLKDRYYSGLIYTYSGLFCVVINPYKNLPIYSENIIEMYRGKKRHEM
PPHIYAISESAYRCMLQDREDQSILCTGESGAGKTENTKKVIQYLAHVAS
SHKGRKDHNIPGELERQLLQANPILESFGNAKTVKNDNSSRFGKFIRINF
DVTGYIVGANIETYLLEKSRAVRQAKDERTFHIFYQLLSGAGEHLKSDLL
LEGFNNYRFLSNGYIPIPGQQDKDNFQETMEAMHIMGFSHEEILSMLKVV
SSVLQFGNISFKKERNTDQASMPENTVAQKLCHLLGMNVMEFTRAILTPR
IKVGRDYVQKAQTKEQADFAVEALAKATYERLFRWLVHRINKALDRTKRQ
GASFIGILDIAGFEIFELNSFEQLCINYTNEKLQQLFNHTMFILEQEEYQ
REGIEWNFIDFGLDLQPCIDLIERPANPPGVLALLDEECWFPKATDKTFV
EKLVQEQGSHSKFQKPRQLKDKADFCIIHYAGKVDYKADEWLMKNMDPLN
DNVATLLHQSSDRFVAELWKDVDRIVGLDQVTGMTETAFGSAYKTKKGMF
RTVGQLYKESLTKLMATLRNTNPNFVRCIIPNHEKRAGKLDPHLVLDQLR
CNGVLEGIRICRQGFPNRIVFQEFRQRYEILTPNAIPKGFMDGKQACERM
IRALELDPNLYRIGQSKIFFRAGVLAHLEEERDLKITDIIIFFQAVCRGY
LARKAFAKKQQQLSALKVLQRNCAAYLKLRHWQWWRVFTKVKPLLQVTRQ
EEELQAKDEELLKVKEKQTKVEGELEEMERKHQQLLEEKNILAEQLQAET
ELFAEAEEMRARLAAKKQELEEILHDLESRVEEEEERNQILQNEKKKMQA
HIQDLEEQLDEEEGARQKLQLEKVTAEAKIKKMEEEILLLEDQNSKFIKE
KKLMEDRIAECSSQLAEEEEKAKNLAKIRNKQEVMISDLEERLKKEEKTR
QELEKAKRKLDGETTDLQDQIAELQAQIDELKLQLAKKEEELQGALARGD
DETLHKNNALKVVRELQAQIAELQEDFESEKASRNKAEKQKRDLSEELEA
LKTELEDTLDTTAAQQELRTKREQEVAELKKALEEETKNHEAQIQDMRQR
HATALEELSEQLEQAKRFKANLEKNKQGLETDNKELACEVKVLQQVKAES
EHKRKKLDAQVQELHAKVSEGDRLRVELAEKASKLQNELDNVSTLLEEAE
KKGIKFAKDAASLESQLQDTQELLQEETRQKLNLSSRIRQLEEEKNSLQE
QQEEEEEARKNLEKQVLALQSQLADTKKKVDDDLGTIESLEEAKKKLLKD
AEALSQRLEEKALAYDKLEKTKNRLQQELDDLTVDLDHQRQVASNLEKKQ
KKFDQLLAEEKSISARYAEERDRAEAEAREKETKALSLARALEEALEAKE
EFERQNKQLRADMEDLMSSKDDVGKNVHELEKSKRALEQQVEEMRTQLEE
LEDELQATEDAKLRLEVNMQAMKAQFERDLQTRDEQNEEKKRLLIKQVRE
LEAELEDERKQRALAVASKKKMEIDLKDLEAQIEAANKARDEVIKQLRKL
QAQMKDYQRELEEARASRDEIFAQSKESEKKLKSLEAEILQLQEELASSE
RARRHAEQERDELADEITNSASGKSALLDEKRRLEARIAQLEEELEEEQS
NMELLNDRFRKTTLQVDTLNAELAAERSAAQKSDNARQQLERQNKELKAK
LQELEGAVKSKFKATISALEAKIGQLEEQLEQEAKERAAANKLVRRTEKK
LKEIFMQVEDERRHADQYKEQMEKANARMKQLKRQLEEAEEEATRANASR
RKLQRELDDATEANEGLSREVSTLKNRLRRGGPISFSSSRSGRRQLHLEG
ASLELSDDDTESKTSDVNETQPPQSE.

Another non-limiting exemplary sequence of myosin is disclosed herein below:

MTDAQMADFG AAAQYLRKSE KERLEAQTRP FDIRTECFVP DDKEEFVKAK
ILSREGGKVIAETENGKTVT VKEDQVLQQN PPKFDKIEDM AMLTFLHEPA
VLFNLKERYA AWMIYTYSGLFCVTVNPYKW LPVYNAEVVA AYRGKKRSEA
PPHIFSISDN AYQYMLTDRE NQSILITGESGAGKTVNTKR VIQYFASIAA
IGDRGKKDNANANKGTLEDQIIQANPALEAFGNAKTVRNDNSSRFGKFIRIHFGATG
KLASADIETYLLEKSRVIFQLKAERNYHIFYQILSNKKPELLDMLLVTNNPYDYAFVS
QGEVS VASIDDSEEL MATDSAFDVL GFTSEEKAGV
YKLTGAIMHYGNMKFKQKQR EEQAEPDGTE DADKSAYLMG LNSADLLKGL
CHPRVKVGNE YVTKGQSVQQ
VYYSIGALAK AVYEKMFNWM VTRINATLET KQPRQYFIGV LDIAGFEIFD
FNSFEQLCINFTNEKLQQFF NHHMFVLEQE EYKKEGIEWT FIDFGMDLQA
CIDLIEKPMG IMSILEEECMFPKATDMTFK AKLYDNHLGK SNNFQKPRNI
KGKPEAHFSL IHYAGTVDYN ILGWLEKNKDPLNETVVGLY QKSSLKLMAT
LFSSYATADT GDSGKSKGGK KKGSSFQTVS ALHRENLNKLMTNLRTTHPH
FVRCIIPNER KAPGVMDNPL VMHQLRCNGV LEGIRICRKG FPNRILYGDF
RQRYRILNPV AIPEGQFIDS RKGAEKLLSS LDIDHNQYKF GHTKVFFKAG
LLGLLEEMRDERLSRIITRI QAQARGQLMR IEFKKIVERR DALLVIQWNI
RAFMGVKNWP WMKLYFKIKPLLKSAETEKE MATMKEEFGR IKETLEKSEA
RRKELEEKMV SLLQEKNDLQ LQVQAEQDNLNDAEERCDQL IKNKIQLEAK
VKEMNERLED EEEMNAELTA KKRKLEDECS ELKKDIDDLELTLAKVEKEK
HATENKVKNL TEEMAGLDEI IAKLTKEKKA LQEAHQQALD DLQAEEDKVN
TLSKSKVKLE QQVDDLEGSL EQEKKVRMDL ERAKRKLEGD LKLTQESIMD
LENDKLQLEEKLKKKEFDIN QQNSKIEDEQ VLALQLQKKL KENQARIEEL
EEELEAERTA RAKVEKLRSDLSRELEEISE RLEEAGGATS VQIEMNKKRE
AEFQKMRRDL EEATLQHEAT AAALRKKHADSVAELGEQID NLQRVKQKLE
KEKSEFKLEL DDVTSNMEQI IKAKANLEKV SRTLEDQANEYRVKLEEAQR
SLNDFTTQRA KLQTENGELS RQLEEKEALI SQLTRGKLSY TQQMEDLKRQ
LEEEGKAKNA LAHALQSARH DCDLLREQYE EETEAKAELQ RVLSKANSEV
AQCRTKYETDAIQRTEELEE AKKKLAQRLQ DAEEAVEAVN AKCSSLEKTK
HRLQNEIEDL MVDVERSNAAAAALDKKQRN FDKILAEWKQ KYEESQSELE
SSQKEARSLS TELFKLKNAY EESLEHLETFKRENKNLQEE ISDLTEQLGE
GGKNVHELEK VRKQLEVEKL ELQSALEEAE ASLEHEEGKILRAQLEFNQI
KAEIERKLAE KDEEMEQAKR NHQRVVDSLQ TSLDAETRSR NEVLRVKKKM
EGDLNEMEIQ LSHANRMAAE AQKQVKSLQS LLKDTQIQLD DAVRANDDLK
ENIAIVERRNNLLQAELEEL RAVVEQTERS RKLADRELIE TSERVQLLHS
QNTSLINQKK KMDADLSQLQSEVEEAVQEC RNAEEKAKKA ITHAAMMAEE
LKKEQDTSAH LERMKKNMEQ TIKDLQHRLDEAEQIALKGG KKQLQKLEAR
VRELEGELEA EQKRNAESVK GMRKSERRIK ELTYQTEEDKKNLLRLQDLV
DKLQLKVKAY KRQAEEAEEQ ANTNLSKFRK VQHELDEAEE RADIAESQVN
KLRAKSRDIG AKQKMHDEE.

As used herein, “CD1d-binding lipid antigens” refers generally to lipid antigens that bind to the non-classical MHC CD1d.

As used herein, “HSP” refers to all isoforms, variants, and fragments thereof of a protein associated with the name “heat shock protein”, or a biological equivalent thereof. In some embodiments, heat shock proteins includes heat shock protein 60. A non-limiting exemplary sequence of heat shock protein 60 associated with UniProt Reference No. P10809-1 is disclosed herein below:

MLRLPTVFRQMRPVSRVLAPHLTRAYAKDVKFGADARALMLQGVDLLADA
VAVTMGPKGRTVIIEQSWGSPKVTKDGVTVAKSIDLKDKYKNIGAKLVQD
VANNTNEEAGDGTTTATVLARSIAKEGFEKISKGANPVEIRRGVMLAVDA
VIAELKKQSKPVTTPEEIAQVATISANGDKEIGNIISDAMKKVGRKGVIT
VKDGKTLNDELEIIEGMKFDRGYISPYFINTSKGQKCEFQDAYVLLSEKK
ISSIQSIVPALEIANAHRKPLVIIAEDVDGEALSTLVLNRLKVGLQVVAV
KAPGFGDNRKNQLKDMAIATGGAVFGEEGLTLNLEDVQPHDLGKVGEVIV
TKDDAMLLKGKGDKAQIEKRIQEIIEQLDVTTSEYEKEKLNERLAKLSDG
VAVLKVGGTSDVEVNEKKDRVTDALNATRAAVEEGIVLGGGCALLRCIPA
LDSLTPANEDQKIGIEIIKRTLKIPAMTIAKNAGVEGSLIVEKIMQSSSE
VGYDAMAGDFVNMVEKGIIDPTKVVRTALLDAAGVASLLTTAEVVVTEIP
KEEKDPGMGAMGGMGGGMGGGMF.

Multiple sclerosis (MS) is also known as “disseminated sclerosis,” “encephalomyelitis disseminate,” or “allergic encephalomyelitis.” MS is an inflammatory disease in which the fatty myelin sheaths around the axons of the brain and spinal cord are damaged, leading to demyelination and scarring as well as a broad spectrum of signs and symptoms. Multiple sclerosis-related disorders include, for example, neuromyelitis optica spectrum disorder (NMO), uveitis, neuropathis pain, and the like.

“Myelin Oligodendrocyte Glycoprotein” (MOG) is a glycoprotein believed to be important in the process of myelinization of nerves in the central nervous system (CNS). In humans this protein is encoded by the MOG gene. It is speculated to serve as a necessary “adhesion molecule” to provide structural integrity to the myelin sheath and is known to develop late on the oligodendrocyte. The GenBank accession numbers NM_001008228.2 and NP_001008229.1 represent the mRNA and protein sequence, respectively, of the MOG gene. The sequence associated with each of these GenBank accession numbers is incorporated by reference for all purposes.

As used herein, the terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia and metastases thereof. A “metastasis” intends the transference of disease-producing organisms or of malignant or cancerous cells to other parts of the body by way of the blood or lymphatic vessels or membranous surfaces. Non-limiting examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

As used herein, the term “diabetes” intends a variable disorder of carbohydrate metabolism caused by a combination of hereditary and environmental factors and is usually characterized by inadequate secretion or utilization of insulin, by excessive urine production, by excessive amounts of sugar in the blood and urine, and by thirst, hunger, and loss of weight. Diabetes is characterized by Type 1 diabetes and Type 2 diabetes. The nonobese diabetic (“NOD”) mouse is an accepted animal model for the study and treatment of diabetes. Type 1 Diabetes (T1D) in mice is associated with autoreactive CD8⁺ T-cells. Nonobese diabetic (NOD) mice develop a form of TID, closely resembling human TID, that results from selective destruction of pancreatic βcells by T-cells recognizing a growing list of autoantigens. Although initiation of T1D clearly requires the contribution of CD4+ cells, there is compelling evidence that T1D is CD8+ T-cell-dependent. It has been discovered that a significant fraction of islet-associated CD8+ cells in NOD mice use CDR3-invariant Vα17-Jα42+ TCRs, referred to as ‘8.3-TCR-like’. These cells, which recognize the mimotope NRP-A7 (defined using combinatorial peptide libraries) in the context of the MHC molecule K^{d}, are already a significant component of the earliest NOD islet CD8+ infiltrates, are diabetogenic, and target a peptide from islet-specific glucose-6-phosphatase catalytic subunit-related protein (IGRP), a protein of unknown function. The CD8+ cells that recognize this peptide (IGRP₂₀₆₋₂₁₄, similar to NRP-A7) are unusually frequent in the circulation (>1/200 CD8+ cells). Notably, progression of insulitis to diabetes in NOD mice is invariably accompanied by cyclic expansion of the circulating IGRP₂₀₆₋₂₁₄-reactive CD8+ pool, and by avid maturation of its islet-associated counterpart. More recently, it has been shown that islet-associated CD8+ cells in NOD mice recognize multiple IGRP epitopes, indicating that IGRP is a dominant autoantigen for CD8+ cells, at least in murine T1D. NOD islet-associated CD8+ cells, particularly those found early on in the disease process also recognize an insulin epitope (Ins B₁₅₋₂₃).

As used herein, the term “pre-diabetes” intends an asymptomatic period preceding a diabetic condition characterized by subclinical beta cell damage wherein the patient exhibits normal plasma glucose levels. It is also characterized by the presence of islet cell autoantibodies (ICAs) and, when close to the onset of clinical symptoms, may be accompanied by intolerance to glucose.

As used herein, the term “multiple sclerosis-related disorder” intends a disorder that co-presents with a susceptibility to MS or with MS. Non-limiting examples of such include neuromyelitis optica spectrum disorder (NMO), uveitis, neuropathis pain sclerosis, atherosclerosis, arteriosclerosis, sclerosis disseminata, systemic sclerosis, spino-optical MS, primary progressive MS (PPMS), and relapsing remitting MS (RRMS), progressive systemic sclerosis, and ataxic sclerosis.

The terms “epitope” and “antigenic determinant” are used interchangeably to refer to a site on an antigen to which B and/or T cells respond or recognize. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-20 amino acids in a unique spatial conformation. Methods of determining spatial conformation of epitopes include, for example, x-ray crystallography and 2-dimensional nuclear magnetic resonance. See, e.g., Glenn E. Morris, Epitope Mapping Protocols (1996). T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., J. Inf. Dis., 170:1110-1119, 1994), by antigen-dependent killing (cytotoxic T lymphocyte assay, Tigges et al., J. Immunol., 156(10):3901-3910, 1996) or by cytokine secretion. The presence of a cell-mediated immunological response can be determined by proliferation assays (CD4⁺ T cells) or CTL (cytotoxic T lymphocyte) assays.

Optionally, an antigen or preferably an epitope of an antigen, can be chemically conjugated to, or expressed as, a fusion protein with other proteins, such as MHC and MHC related proteins.

As used herein, the terms “patient” and “subject” are used synonymously and refer to a mammal. In some embodiments, the patient is a human. In other embodiments, the patient is a mammal in need of veterinary medicine or is a mammal commonly used in a laboratory. In some embodiments, the mammal is a mouse, rat, simian, canine, feline, bovine, equine, or ovine.

As used in this disclosure, the term “polynucleotide” refers to a nucleic acid molecule that either is recombinant or has been isolated free of total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be RNA, DNA, analogs thereof, or a combination thereof. A nucleic acid encoding all or part of a polypeptide may contain a contiguous nucleic acid sequence encoding all or a portion of such a polypeptide of the following lengths: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs. It is also contemplated that a particular polypeptide from a given species may be encoded by nucleic acids containing natural variations that have slightly different nucleic acid sequences but, nonetheless, encode the same or substantially similar protein, polypeptide, or peptide.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “isolated” or “recombinant” as used herein with respect to nucleic acids, such as DNA or RNA, refers to molecules separated from other DNAs or RNAs, respectively, that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example, those described in Current Protocols in Molecular Biology (Ausubel et al., eds. 1987) Supplement 30, section 7.7.18, Table 7.7.1. Preferably, default parameters are used for alignment. A certain alignment program is BLAST, using default parameters. In particular, certain programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

It is to be inferred without explicit recitation and unless otherwise intended, that when the present disclosure relates to an antigen, polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference antigen, protein, antibody, fragment, polypeptide or nucleic acid, and intends those having minimal homology while still maintaining the desired structure or functionality. Unless specifically recited herein, it is contemplated that any polynucleotide, polypeptide or protein mentioned herein also includes equivalents thereof. In one aspect, an equivalent polynucleotide is one that hybridizes under stringent conditions to the polynucleotide or complement of the polynucleotide as described herein for use in the described methods. In another aspect, an equivalent antibody or antigen binding polypeptide intends one that binds with at least 70%, or alternatively at least 75%, or alternatively at least 80%, or alternatively at least 85%, or alternatively at least 90%, or alternatively at least 95% affinity or higher affinity to a reference antibody or antigen binding fragment. In another aspect, the equivalent thereof competes with the binding of the antibody or antigen-binding fragment to its antigen under a competitive ELISA assay. In another aspect, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a polymerase chain (PC) reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions.

As used herein, the terms “treating,” “treatment” and the like are used herein to mean obtaining a desired pharmacologic and/or physiologic effect. The effect may be therapeutic in terms of a partial or complete cure for a disorder and/or adverse effect attributable to the disorder. In one aspect, treatment indicates a reduction in the signs of the disease using an established scale.

As used herein, the term “treatment” or “treating” as it relates to oncology, means any treatment of a disease or condition or associated disorder, in a patient, including inhibiting the disease or condition, that is, arresting or suppressing the development of clinical symptoms, such as cachexia in cancer; and/or relieving the disease or condition that is causing the regression of clinical symptoms, e.g., increasing overall survival or reducing tumor burden.

In some aspects, the term “treating” refers to an improvement in clinical outcomes. The term “clinical outcome” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect. “Overall Survival” (OS) intends a prolongation in life expectancy as compared to naive or untreated individuals or patients. “Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease. “Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer. “Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up. “Relative Risk” (RR), in statistics and mathematical epidemiology, refers to the risk of an event (or of developing a disease) relative to exposure. Relative risk is a ratio of the probability of the event occurring in the exposed group versus a non-exposed group.

A “composition” is intended to mean a combination of active agent and another compound or composition, inert (for example, a detectable agent or label) or active, such as an adjuvant. In certain embodiments, the composition does not contain an adjuvant.

A “pharmaceutical composition” is intended to include the combination of an active agent with a carrier, inert or active, making the composition suitable for diagnostic or therapeutic use in vitro, in vivo or ex vivo.

The term “functionally equivalent codon” is used herein to refer to codons that encode the same amino acid, such as the six codons for arginine or serine, and also refers to codons that encode biologically equivalent amino acids (see Table 2).

As used herein, a “protein” or “polypeptide” or “peptide” refers to a molecule comprising at least five amino acid residues.

Other objects, features and advantages of the present disclosure will become apparent from the following detailed description. Additional definitions are also provided therein. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### DESCRIPTIVE EMBODIMENTS

Autoimmune diseases such as type 1 diabetes (T1D), multiple sclerosis and rheumatoid arthritis result from chronic autoimmune responses involving T cells and B cells recognizing numerous antigenic epitopes on incompletely defined lists of autoantigens (Santamaria, P. (2010) Immunity 32:437-445; Babbe, H. et al. (2000) J. Exp. Med. 192:393-404; Firestein, G. S. (2003) Nature 423:356-361). Eliminating or suppressing all polyclonal autoreactive T-cell specificities (known and unknown) in each individual autoimmune disorder without compromising systemic immunity is not currently possible.

Adoptive transfer of polyclonal FOXP3⁺CD4⁺CD25⁺ regulatory T (T_{reg}) cells expanded ex vivo has been proposed as an alternative therapeutic approach (Sakaguchi, S. et al. (2006) Immunol. Rev. 212:8-27). The potential for bystander immunosuppression, the lack of effective strategies for expanding antigen-specific T_{reg} cells in vitro, and the lineage instability of FOXP3⁺ T_{reg} cells, have hindered the clinical translation of this approach (Zhou, X. et al. (2009) Nature Immunol. 10:1000-1007; Komatsu, N. et al. (2014) Nature Med. 20:62-68; Bailey-Bucktrout, S. L. et al. (2013) Immunity 39:949-962). T_{R}1 FOXP3⁻CD4⁺CD25⁻ T cells, which produce the cytokines IL-10 and IL-21, and express the surface markers CD49b and LAG-3 and the transcription factor c-Maf 8, constitute another regulatory T-cell subset recently exploited for the treatment of human inflammatory diseases (McLarnon, A. (2012) Nature Rev. Gastroenterol. Hepatol. 9:559; Desreumaux, P. et al. (2012) Gastroenterology 143:1207-1217; Roncarolo, M. G. et al. (2011) Immunol. Rev. 241:145-163). However, as with FOXP3+ Treg cells, there are no pharmacological approaches that can expand autoantigen- or disease-specific T_{R}1-like cells in vivo.

Thus, regulatory T cells hold promise as targets for therapeutic intervention in autoimmunity, but approaches capable of expanding antigen-specific regulatory T cells in vivo are currently not available. Here Applicant shows that systemic delivery of nanoparticles coated with autoimmune-disease-relevant peptides bound to major histocompatibility complex class II (pMHCII) molecules triggers the generation and expansion of antigen-specific regulatory CD4⁺ T cell type 1 (T_{R}1)-like cells in different mouse models, including mice humanized with lymphocytes from patients, leading to resolution of established autoimmune phenomena. Ten pMHCII-based nanomedicines show similar biological effects, regardless of genetic background, prevalence of the cognate T-cell population or MHC restriction. These nanomedicines promote the differentiation of disease-primed autoreactive T cells into T_{R}1-like cells, which in turn suppress autoantigen-loaded antigen-presenting cells and drive the differentiation of cognate B cells into disease-suppressing regulatory B cells, without compromising systemic immunity. pMHCII-based nanomedicines thus represent a new class of drugs, potentially useful for treating a broad spectrum of autoimmune conditions in a disease-specific manner.

Applicant previously discovered that systemic delivery of nanoparticles (NPs) coated with T1D-relevant pMHC class I complexes (pMHC-NPs) could blunt the progression of T1D by expanding subsets of CD8⁺ T cells with regulatory potential but conventional memory-like phenotype (Tsai, S. et al. (2010) Immunity 32:568-580). As the nanoparticles could be coated with different pMHC class I complexes, Applicant reasoned that pMHC-NP therapy may utilize a naturally occurring negative feedback regulatory loop, whereby chronic autoantigenic exposure (and exposure to pMHC-NPs) could trigger the differentiation of autoreactive T cells into regulatory T-cell progeny. By this reasoning, Applicant predicted and has shown herein that NPs coated with disease-relevant pMHCII complexes might be able to expand disease-specific regulatory CD4⁺ T cells in vivo.

This disclosure builds on those initial observations by providing pMHC-NPs, compositions and methods for making them, as well as their use.

### Substrates/Particles

By “particle,” “nanoparticle,” “microparticle,” “bead,” “microsphere,” and grammatical equivalents herein is meant small discrete particles that are administrable to a subject. In certain embodiments, the particles are substantially spherical in shape. The term “substantially spherical,” as used herein, means that the shape of the particles does not deviate from a sphere by more than about 10%. Various known antigen or peptide complexes of the disclosure may be applied to the particles.

The nanoparticle core of the pMHC-NP comprises, or consists essentially of, or yet further consists of a core, for example a solid core, a metal core, a dendrimer core, a polymeric micelle nanoparticle core, a nanorod, a fullerene, a nanoshell, a coreshell, a protein-based nanostructure or a lipid-based nanostructure. In some aspects, the nanoparticle core is bioabsorbable and/or biodegradable. In some aspects, the nanoparticle core is a dendrimer nanoparticle core comprising, or alternatively consisting essentially thereof, or yet further consisting of a highly branched macromolecule having a tree-like structure growing from a core. In further aspects, the dendrimer nanoparticle core may comprise, or alternatively consist essentially thereof, or yet further consist of a poly(amidoamine)-based dendrimer or a poly-L-lysine-based dendrimer. In certain aspects, the nanoparticle core is a polymeric micelle core comprising, or alternatively consisting essentially thereof, or yet further consisting of an amphiphilic block co-polymer assembled into a nano-scaled core-shell structure. In further aspects, the polymeric micelle core comprises, or alternatively consists essentially thereof, or yet further consists of a polymeric micelle produced using polyethylene glycol-diastearoylphosphatidylethanolamine block copolymer. In a further aspect, the nanoparticle core comprises, or alternatively consists essentially of, or yet further consists of a metal. In another aspect, the nanoparticle core is not a liposome. Additional examples of core materials include but are not limited to, standard and specialty glasses, silica, polystyrene, polyester, polycarbonate, acrylic polymers, polyacrylamide, polyacrylonitrile, polyamide, fluoropolymers, silicone, celluloses, silicon, metals (e.g., iron, gold, silver), minerals (e.g., ruby), nanoparticles (e.g., gold nanoparticles, colloidal particles, metal oxides, metal sulfides, metal selenides, and magnetic materials such as iron oxide), and composites thereof. In some embodiments, an iron oxide nanoparticle core comprises iron (II, III) oxide. The core could be of homogeneous composition, or a composite of two or more classes of material depending on the properties desired. In certain aspects, metal nanoparticles will be used. These metal particles or nanoparticles can be formed from Au, Pt, Pd, Cu, Ag, Co, Fe, Ni, Mn, Sm, Nd, Pr, Gd, Ti, Zr, Si, and In, precursors, their binary alloys, their ternary alloys and their intermetallic compounds. See U.S. Pat. No. 6,712,997, which is incorporated herein by reference in its entirety. In certain embodiments, the compositions of the core and layers (described below) may vary provided that the nanoparticles are biocompatible and bioabsorbable. The core could be of homogeneous composition, or a composite of two or more classes of material depending on the properties desired. In certain aspects, metal nanospheres will be used. These metal nanoparticles can be formed from Fe, Ca, Ga and the like. In certain embodiments, the nanoparticle comprises, or alternatively consists essentially of, or yet further consists of a core comprising metal or metal oxide such as gold or iron oxide.

The particles typically consist of a substantially spherical core and optionally one or more layers or coatings. The core may vary in size and composition as described herein. In addition to the core, the particle may have one or more layers to provide functionalities appropriate for the applications of interest. The thicknesses of layers, if present, may vary depending on the needs of the specific applications. For example, layers may impart useful optical properties.

Layers may also impart chemical or biological functionalities, referred to herein as chemically active or biologically active layers. These layers typically are applied on the outer surface of the particle and can impart functionalities to the pMHC-NPs. The layer or layers may typically range in thickness from about 0.001 micrometers (1 nanometer) to about 10 micrometers or more (depending on the desired particle diameter) or from about 1 nm to 5 nm, or alternatively from about 1 nm to about 10 nm, or alternatively from about 1 nm to about 40 nm, or from about 15 nm to about 25 nm, or about 20 nm, and ranges in between.

The layer or coating may comprise, or alternatively consist essentially of, or yet further consist of a biodegradable sugar or other polymer. Examples of biodegradable layers include but are not limited to dextran; poly(ethylene glycol); poly(ethylene oxide); mannitol; poly(esters) based on polylactide (PLA), polyglycolide (PGA), polycaprolactone (PCL); poly(hydroxalkanoate) of the PHB-PHV class; and other modified poly(saccharides) such as starch, cellulose and chitosan. Additionally, the nanoparticle may include a layer with suitable surfaces for attaching chemical functionalities for chemical binding or coupling sites.

Layers can be produced on the nanoparticles in a variety of ways known to those skilled in the art. Examples include sol-gel chemistry techniques such as described in Iler, Chemistry of Silica, John Wiley & Sons, 1979; Brinker and Scherer, Sol-gel Science, Academic Press, (1990). Additional approaches to producing layers on nanoparticles include surface chemistry and encapsulation techniques such as described in Partch and Brown, J. Adhesion, 67:259-276, 1998; Pekarek et al., Nature, 367:258, (1994); Hanprasopwattana, Langmuir, 12:3173-3179, (1996); Davies, Advanced Materials, 10:1264-1270, (1998); and references therein. Vapor deposition techniques may also be used; see, for example, Golman and Shinohara, Trends Chem. Engin., 6:1-6, (2000); and U.S. Pat. No. 6,387,498. Still other approaches include layer-by-layer self-assembly techniques such as described in Sukhorukov et al., Polymers Adv. Tech., 9(10-11):759-767, (1998); Caruso et al., Macromolecules, 32(7):2317-2328, (1998); Caruso et al., J. Amer. Chem. Soc., 121(25):6039-6046, (1999); U.S. Pat. No. 6,103,379 and references cited therein.

In some aspects, the nanoparticle core is a dendrimer nanoparticle core comprising, or alternatively consisting essentially thereof, or yet further consisting of a highly branched macromolecule having a tree-like structure growing from a core. In further aspects, the dendrimer nanoparticle may comprise, or alternatively consist essentially thereof, or yet further consist of a poly(amidoamine)-based dendrimer or a poly-L-lysine-based dendrimer. In certain aspects, the nanoparticle core is a polymeric micelle core comprising, or alternatively consisting essentially thereof, or yet further consisting of an amphiphilic block co-polymer assembled into a nano-scaled core-shell structure. In further aspects, the polymeric micelle core may comprise, or alternatively consist essentially thereof, or yet further consist of a polymeric micelle produced using polyethylene glycol-diastearoylphosphatidylethanolamine block copolymer. The dendrimer core or polymeric micelle core may further comprise an outer coating or layer as described herein.

In certain embodiments, specific means of synthesis of dendrimer nanoparticles or nanoparticles with a dendrimer nanoparticle core may require that metal ions are extracted into the interior of dendrimers and then subsequently chemically reduced to yield nearly size-monodispersed particles having dimensions of less than 3 nm, such as the method disclosed in Crooks et al., “Synthesis, Characterization, and Applications of Dendrimer-Encapsulated Nanoparticles”. The Journal of Physical Chemistry B (109): 692-704 (2005), wherein the resulting dendrimer core component serves not only as a template for preparing the nanoparticle but also to stabilize the nanoparticle, making it possible to tune solubility, and provides a means for immobilization of the nanoparticle on solid supports.

The size of the nanoparticle core can range from about 1 nm to about 1 μm. In certain embodiments, the nanoparticle core is less than about 1 μm in diameter. In other embodiments, the nanoparticle core is less than about 500 nm, less than about 400 nm, less than about 300 nm, less than about 200 nm, less than about 100 nm, or less than about 50 nm in diameter. In further embodiments, the nanoparticle core is from about 1 nm to about 10 nm, 15 nm, 20 nm, 25 nm, 30 nm, 40 nm, 50 nm, 75 nm, or 100 nm in diameter. In specific embodiments, the nanoparticle core has a diameter of from about 1 nm to about 100 nm; from about 1 nm to about 75 nm; from about 1 nm to about 50 nm; from about 1 nm to about 25 nm; from about 1 nm to about 25 nm; from about 5 nm to about 100 nm; from about 5 nm to about 50 nm; or from about 5 nm to about 25 nm, or from about 15 nm to about 25 nm, or about 20 nm. In some embodiments, the nanoparticles core has a diameter of from about 25 nm to about 60 nm, or from about 25 nm to about 50 nm, or from about 20 nm to about 40 nm, or from about 15 nm to about 50 nn, or from about 15 nm to about 40 nm, or from about 15 nm to about 35 nm, or from about 15 nm to about 30 nm, or from about 15 nm to about 25 nm, or alternatively about 15 nm, or about 20 nm, or about 25 nm, or about 30 nm, or about 35 nm, or about 40 nm.

The size of the pMHC-NP, with or without the layer, can range from about 5 nm to about 1 μm in diameter. In certain embodiments, the pMHC-NP complex is less than about 1 μm or alternatively less than 100 nm in diameter. In other embodiments, the pMHC-NP complex is less than about 500 nm, less than about 400 nm, less than about 300 nm, less than about 200 nm, less than about 100 nm, or less than about 50 nm in diameter. In further embodiments, the complex is from about 5 nm or 10 nm to about 50 nm, or from about 5 nm to about 75 nm, or from about 5 nm to about 50 nm, or from about 5 nm to about 60 nm, or from about 10 nm to about 50 nm, or from about 10 nm to about 60 nm, or from about 10 nm to about 70 nm, or from about 10 nm to about 75 nm, or from about 20 nm to about 50 nm, or from about 20 nm to about 60 nm, or from about 20 nm to about 70 nm, or from about 20 nm to about 75 nm, or from about 30 nm to about 50 nm, or from about 30 nm to about 60 nm, or from about 30 nm to about 70 nm, or from about 30 nm to about 75 nm, or in one aspect about 55 nm in diameter. In specific embodiments, the pMHC-NP complex is from about 35 nm to about 60 nm, or from about 35 nm to about 70 nm, or from about 35 nm to about 75 nm in diameter. In one aspect, the pMHC-NP complex is from about 30 nm to about 50 nm in diameter.

### Antigen-MHC Complexes

The nanoparticle complexes of this disclosure comprise a nanoparticle core, with or without a layer, coupled to an antigen-MHC (pMHC) complex. The selection of antigen will depend on the disease or condition to be treated, as noted above. The individual polypeptide (e.g., MHC) and the antigenic (e.g., peptide) components form a complex through covalent or non-covalent binding (e.g. through hydrogen bonds, ionic bonds, or hydrophobic bonds). The preparation of such complexes may require varying degrees of manipulation and such methods are well known in the literature. In some aspects, antigenic components can be associated non-covalently with the pocket portion of the MHC component by, for instance, mixing the MHC and antigenic components; this relies on the natural binding affinity between an MHC and an antigen. Alternatively, in some aspects, the MHC component may be covalently bound to the antigenic component using standard procedures, such as, but not limited to, the introduction of known coupling agents or photo affinity labelling (see e.g., Hall et al., Biochemistry 24:5702-5711 (1985)). In certain aspects, an antigenic component may be operatively coupled to the MHC component via peptide linkages or other methods discussed in the literature, including but not limited to, attachment via carbohydrate groups on the glycoproteins, including, e.g., the carbohydrate moieties of the alpha- and/or beta-chains. In particular embodiments, the antigenic component may be attached to the N-terminal or C-terminal end of an appropriate MHC molecule. Alternatively, in certain embodiments, the MHC complex may be recombinantly formed by incorporating the sequence of the antigenic component into a sequence encoding an MHC, such that both retain their functional properties.

Multiple antigen-MHC complexes may be coupled to the same nanoparticle core; these complexes, MHCs, and/or antigens may be the same or different from one another.

Valency is defined as the number of pMHC complexes per nanoparticle core. In certain embodiments the valency of the nanoparticle may range between about 1 pMHC complex to 1 nanoparticle core to about 6000 pMHC complexes to 1 nanoparticle core, or alternatively between about 10:1 to about 6000:1, or alternatively between about 11:1 to about 6000:1, or alternatively between about 12:1 to about 6000:1, or alternatively at least 2:1, or alternatively at least 8:1, or alternatively at least 9:1, or alternatively at least 10:1, or alternatively at least 11:1, or alternatively at least 12:1.

In some aspects, the valency is from about 10:1 to about 6000:1, or from about 20:1 to about 5500:1, or alternatively from about 10:1 to about 5000:1, or alternatively from about 10:1 to about 4000:1, or alternatively from about 10:1 to about 3500:1, or alternatively from about 10:1 to about 3000:1, or alternatively from about 10:1 to about 2500:1, or alternatively from about 10:1 to about 2000:1, or alternatively from about 10:1 to about 1500:1, or alternatively from about 10:1 to 1000:1, or alternatively from about 10:1 to about 500:1, or alternatively from about 10:1 to about 100:1, or alternatively from about 20:1 to about 50:1, or alternatively from about 25:1 to about 60:1; alternatively from about 30:1 to about 50:1, or alternatively from about 35:1 to about 45:1, or alternatively about 40:1.

Applicant has discovered that pMHC density on the nanoparticle regulates the ability of the pMHC-NPs to trigger or differentiate T_{R}1 cell formation in a dose-independent manner. Density is calculated as the number of complexes per unit surface area of the nanoparticle. The surface area of the nanoparticle may be determined with or without the layers, including, but not limited to, linkers that conjugate the pMHC complex to the nanoparticle. For the purposes of calculating density, the relevant surface area value is based on the final diameter of the particle construct without the pMHC complex, with or without the outer layer on the nanoparticle core.

It is determined and disclosed herein that the density of the pMHC complexes on the nanoparticle contributes to the therapeutic benefit in a dose-independent manner. Thus, as disclosed herein, the nanoparticle can have a defined pMHC density in the range of from about 0.01 pMHC, or alternatively 0.025 pMHC, molecules per 100 nm² of surface area of the nanoparticle including the layer or complex, assuming at least 2 MHC molecules, or alternatively at least 8, or alternatively at least 9, or alternatively at least 10, or alternatively at least 11, or alternatively at least 12, pMHC molecules complexed to the nanoparticle to about 100 pMHC molecules per 100 nm² of surface area. In one aspect, the nanoparticle has a density of pMHC from about 0.05 pMHC per 100 nm² to about 76 pMHC/100 nm², or alternatively from 0.1 pMHC/100 nm² to about 50 pMHC/100 nm², or alternatively from about 0.3 pMHC/100 nm² to about 25 pMHC/100 nm², or alternatively from about 0.35 pMHC/100 nm² to about 25 pMHC/100 nm², or alternatively from about 0.4 pMHC/100 nm² to about 50 pMHC/100 nm², or alternatively from about 0.4 pMHC/100 nm² to about 25 pMHC/100 nm², or alternatively from about 0.4 pMHC/100 nm² to about 20 pMHC/100 nm², 0.4 pMHC/100 nm² to about 10 pMHC/100 nm², 0.4pMHC/100 nm² to about 5 pMHC/100 nm², or alternatively from about 0.5 pMHC/100 nm² to about 20 pMHC/100 nm², or alternatively from about 0.5 pMHC/100 nm² to about 10 pMHC/100 nm², or alternatively from about 0.6 pMHC/100 nm² to about 20 pMHC/100 nm², or alternatively from about 1.0 pMHC/100 nm² to about 20 pMHC/100 nm², or alternatively from about 10 pMHC/100 nm² to about 20 pMHC/100 nm², or alternatively at least about 0.4, or alternatively at least about 0.406, or alternatively at least about 0.5, or alternatively at least about 1.0, or alternatively at least about 5.0, or alternatively at least about 10.0, or alternatively at least about 15.0 pMHC/100 nm², or alternatively less than about 76 pMHC/100 nm², or alternatively less than about 50 pMHC/100 nm², or alternatively less than about 47.75 pMHC/100 nm² or alternatively less than about 25 pMHC/100 nm², or alternatively less than about 20 pMHC/100 nm².

In certain embodiments, the pMHC density per nanoparticle is from about 0.4 pMHC/100 nm² to about 25 pMHC/100 nm², or from about 0.4 pMHC/100 nm² to about 20 pMHC/100 nm², or from about 0.4 pMHC/100 nm² to about 15 pMHC/100 nm², or from about 0.4 pMHC/100 nm² to about 14 pMHC/100 nm², or from about 0.4 pMHC/100 nm² to about 13 pMHC/100 nm², or from about 0.4 pMHC/100 nm² to about 12 pMHC/100 nm², or from about 0.4 pMHC/100 nm² to about 11.6 pMHC/100 nm², or from about 0.4 pMHC/100 nm² to about 11.5 pMHC/100 nm², or from about 0.4 pMHC/100 nm² to about 11 pMHC/100 nm², or from about 0.4 pMHC/100 nm² to about 10 pMHC/100 nm², or from about 0.4 pMHC/100 nm² to about 9 pMHC/100 nm², or from about 0.4 pMHC/100 nm² to about 8 pMHC/100 nm², or from about 0.4 pMHC/100 nm² to about 7 pMHC/100 nm², or from about 0.4 pMHC/100 nm² to about 6 pMHC/100 nm², or from about 0.4 pMHC/100 nm² to about 5 pMHC/100 nm², or from about 0.4 pMHC/100 nm² to about 4 pMHC/100 nm², or from about 0.4 pMHC/100 nm² to about 3 pMHC/100 nm², or from about 0.4 pMHC/100 nm² to about 2.5 pMHC/100 nm², or from about 0.4 pMHC/100 nm² to about 2 pMHC/100 nm², or from about 0.4 pMHC/100 nm² to about 1.5 pMHC/100 nm².

In yet further embodiments, the nanoparticle may have a pMHC density of from about 0.22 pMHC/100 nm² to about 10 pMHC/100 nm², or from about about 0.22 pMHC/100 nm² to about 9 pMHC/100 nm², or from about about 0.22 pMHC/100 nm² to about 8 pMHC/100 nm², or from about about 0.22 pMHC/100 nm² to about 7 pMHC/100 nm², or from about about 0.22 pMHC/100 nm² to about 6 pMHC/100 nm², or from about about 0.22 pMHC/100 nm² to about 5 pMHC/100 nm², or from about about 0.22 pMHC/100 nm² to about 4 pMHC/100 nm², or from about about 0.22 pMHC/100 nm² to about 3 pMHC/100 nm², or from about about 0.22 pMHC/100 nm² to about 2 pMHC/100 nm², or from about about 0.22 pMHC/100 nm² to about 1.5 pMHC/100 nm². In some aspects, the nanoparticle has a pMHC density of from about 0.22 pMHC/100 nm² to about 10 pMHC/100 nm², or 0.24 pMHC/100 nm² to about 9 pMHC/100 nm², or from about 0.26 pMHC/100 nm² to about 8 pMHC/100 nm², or from about 0.28 pMHC/100 nm² to about 7 pMHC/100 nm², or from about 0.24pMHC/100 nm² to about 4 pMHC/100 nm², or from about 0.5 pMHC/100 nm² to about 3 pMHC/100 nm², or from about 0.6 pMHC/100 nm² to about 1.5 pMHC/100 nm². In some embodiments, the nanoparticle has a pMHC density of from about 0.4 pMHC/100 nm² to about 1.3 pMHC/100 nm², or alternatively from about 0.5 pMHC/100 nm² to about 0.9 pMHC/100 nm², or alternatively from about 0.6 pMHC/100 nm² to about 0.8 pMHC/100 nm².

In yet further embodiments, the nanoparticle can have a density of from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 pMHC/100 nm². In specific embodiments, the nanoparticle has a density of from about 0.4 pMHC/100 nm² to about 1.5 pMHC/100 nm² or from about 0.4 pMHC/100 nm² to about 6 pMHC/100 nm² or from about 0.4 pMHC/100 nm² to about 11 pMHC/100 nm².

In some aspects, provided herein is a complex comprising a nanoparticle core, wherein a plurality of disease-relevant antigen-MHC (pMHC) complexes are coupled to the core; the diameter of the core is from about 15 nm to about 25 nm; and wherein the pMHC density on the nanoparticle is from about 0.4 pMHC/100 nm² to about 6 pMHC/100 nm² of the surface area of the nanoparticle. In some embodiments, the complex further comprises an outer layer on the nanoparticle core, wherein the pMHC complex is coupled to the nanoparticle core and/or the outer layer, and wherein the diameter of the nanoparticle core and the outer layer is from about 35 nm to about 45 nm.

The term “operatively coupled” or “coated” as used herein, refers to a situation where individual polypeptide (e.g., MHC) and antigenic (e.g., peptide) components are combined to form the active complex prior to binding at the target site, for example, an immune cell. This includes the situation where the individual polypeptide complex components are synthesized or recombinantly expressed and subsequently isolated and combined to form a complex, in vitro, prior to administration to a subject; the situation where a chimeric or fusion polypeptide (i.e., each discrete protein component of the complex is contained in a single polypeptide chain) is synthesized or recombinantly expressed as an intact complex. Typically, polypeptide complexes are added to the nanoparticles to yield nanoparticles with adsorbed or coupled polypeptide complexes having a ratio of number of molecules:number of nanoparticle from about, at least about or at most about 0.1, 0.5, 1, 3, 5, 7, 10, 15, 20, 25, 30, 35, 40, 50, 100, 125, 150, 175, 200, 225, 250, 275, 300, 325, 350, 375, 400, 425, 450, 475, 500, 600, 700, 800, 900, 1000, 1500 or more to: 1, more typically 0.1:1, 1:1 to 50:1 or 300:1, and ranges therebetween where the ratios provide the selected endpoints of each range. The polypeptide content of the nanoparticles can be determined using standard techniques.

### MHC Molecules

As used herein and unless specifically noted, the term MHC in the context of an pMHC complex intends a classical or a non-classical MHC class I protein and/or or classical or non-classical MHC class II protein, any loci of HLA DR, HLA DQ, HLA DP, HLA-A, HLA-B, HLA-C, HLA-E, CD1d, or a fragment or biological equivalent thereof, dual or single chain constructs, dimers (Fc fusions), tetramers, multimeric forms, and a polymeric form of MHCI or MHCII. In some embodiments, the pMHC can be a single chain construct. In some embodiments, the pMHC can be a dual-chain construct.

In some embodiments, the MHC protein can be a dimer or a multimer.

In some embodiments, the MHC protein may comprise a knob-in-hole based MHC-alpha-Fc/MHC-beta-Fc heterodimer or multimer.

As noted above, “knob-in-hole” is a polypeptidyl architecture requiring a protuberance (or “knob”) at an interface of a first polypeptide and a corresponding cavity (or a “hole”) at an interface of a second polypeptide, such that the protuberance can be positioned in the cavity so as to promote heteromultimer formation. Protuberances are constructed by replacing small amino acid side chains from the interface of the first polypeptide with larger side chains (e.g., phenylalanine or tyrosine). Cavities of identical or similar size to the protuberances are created in the interface of the second polypeptide by replacing large amino acid side chains with smaller ones (e.g., alanine or threonine). The protuberances and cavities can be made by synthetic means such as by altering the nucleic acid encoding the polypeptides or by peptide synthesis, using routine methods by one skilled in the art. In some embodiments, the interface of the first polypeptide is located on an Fc domain in the first polypeptide; and the interface of the second polypeptide is located on an Fc domain on the second polypeptide.

As noted above, “MHC-alpha-Fc/MHC-beta-Fc” is a heterodimer comprising a first polypeptide and a second polypeptide, wherein the first polypeptide comprises an MHC class II α-chain and an antibody Fc domain; the second polypeptide comprises an MHC class II β-chain and an antibody Fc domain. A knob-in-hole MHC-alpha-Fc/MHC-beta-Fc further requires that the Fc domains of each polypeptide interface with one another through the complementary positioning of a protuberance on one Fc domain within the corresponding cavity on the other Fc domain.

In certain embodiments of the disclosure, a particular antigen is identified and presented in the antigen-MHC-nanoparticle complex in the context of an appropriate MHC class I or II polypeptide. Presentation of antigens to T cells is mediated by two distinct classes of molecules, MHC class I (MHC-I) and MHC class II (MHC-II), which utilize distinct antigen processing pathways. Peptides derived from intracellular antigens are presented to CD8⁺ T cells by MHC class I molecules, which are expressed on virtually all cells, while extracellular antigen-derived peptides are presented to CD4⁺ T cells by MHC-II molecules. However, there are certain exceptions to this dichotomy. Several studies have shown that peptides generated from endocytosed particulate or soluble proteins are presented on MHC-I molecules in macrophages as well as in dendritic cells. In certain aspects, the genetic makeup of a subject may be assessed to determine which MHC polypeptide is to be used for a particular patient and a particular set of peptides. In certain embodiments, the MHC class 1 component may comprise, consist essentially of, or alternatively further consist thereof all or part of a HLA-A, HLA-B, HLA-C, HLA-E, HLA-F, HLA-G or CD-1 molecule. In embodiments wherein the MHC component is a MHC class II component, the MHC class II component may comprise, consist essentially of, or alternatively further consist thereof all or a part of a HLA-DR, HLA-DQ, or HLA-DP. In certain embodiments, the MHC may comprise HLA DRB1, HLA DRB3, HLA DRB4, HLA DRB5, HLA DQB1, HLA DQA1, IAg₇, I-Ab, I-Ad, HLA-DQ, HLA-DP, HLA-A, HLA-B, HLA-C, HLA-E or CD1d.

Non-classical MHC molecules are also contemplated for use in MHC complexes of the disclosure. In some embodiments, non-classical MHC molecules are non-polymorphic, conserved among species, and possess narrow, deep, hydrophobic ligand binding pockets. These binding pockets are capable of presenting glycolipids and phospholipids to Natural Killer T (NKT) cells. NKT cells represent a unique lymphocyte population that co-express NK cell markers and a semi-invariant T cell receptor (TCR). They are implicated in the regulation of immune responses associated with a broad range of diseases.

As noted above, the term “MHC” may be used interchangeably with the term “human leukocyte antigen” (HLA) when used in reference to human MHC; thus, MHC refers to all HLA subtypes including, but not limited to, the classical MHC genes disclosed above: HLA-A, HLA-B, HLA-C, HLA-DP, HLA-DQ, and HLA-DR, in addition to all variants, isoforms, isotypes, and other biological equivalents thereof.

MHCs for use according to the present disclosure may be produced, isolated, or purified through techniques known in the art. Common protocols for obtaining MHCs involve steps such as, but not limited to, electrophoresis or other techniques of charge or size based separation, biotinylation or other tagging methods and purification, or transfection and induction of vector constructs expressing MHC proteins. Purified animal antibodies are also available through commercially available sources, including retailers such as eBioscience, Biolegend, or Tonbo Biosciences.

In certain embodiments, the MHC of the antigen-MHC complexes may be classical MHCI, non-classical MHCI, classical MHCII, non-classical MHCII, dimers (Fc fusions), MHC tetramers, or a polymeric form of MHC. In some embodiments, MHC multimers are generated according to methods well documented in the art, see, e.g., Bakker et al. “MHC Multimer Technology: Current Status and Future Prospects,” Current Opinion in Immunology, Vol. 17, No. 4 pp. 428-433 (2005) and references cited therein. Non-limiting exemplary methods include the use of a biotinylating agent such as, but not limited to, streptavidin or avidin, to bind MHC monomers, creating a multimeric structure with the agent as a backbone. MHC dimers, specifically, may alternatively be produced through fusion with antibody constant regions or Fc regions; this may be accomplished through operative coupling directly or through a linker, e.g. a cysteine linker.

### Co-Stimulatory Molecule Components

In certain aspects, the NPs additionally comprise, or alternatively consist essentially of, or yet further consist of at least one co-stimulatory molecule. Co-stimulatory molecules are molecules that produce a secondary signal in vivo that serves to activate naïve T cells into antigen-specific T cells capable of producing an immune response to cells possessing said specific antigen. The present disclosure is not limited to any specific co-stimulatory molecule. The various co-stimulatory molecules are well-known in the art. Some non-limiting examples of co-stimulatory molecules are 4-IBBL, OX40L, CD40, IL-15/IL-15Ra, CD28, CD80, CD86, CD30L, and ICOSL. Only one specific co-stimulatory molecule may be coupled to one nanoparticle or a variety of co-stimulatory molecules may be coupled to the same nanoparticle. In certain embodiments, the co-stimulatory molecule is a protein such as an antibody that is capable of agonizing a co-stimulatory receptor on a T cell. In this case, the antibody is capable of inducing a co-stimulatory signal that is necessary to activate naïve T cells and induce an immune response in an antigen-specific manner. Additionally or alternatively, the term “co-stimulatory molecule” as used herein may also refer to an agent capable of generating a co-stimulatory signal by having an agonistic effect on a native co-stimulatory signaling molecule, e.g. anti-CD28 or CD28 ligand generating a CD28 co-stimulatory response.

In specific embodiments, the co-stimulatory molecules of the present disclosure may be any one or more of the following molecules B7-1/CD80, BTLA, B7-2/CD86, CD28, B7-H1/PD-L1, CTLA-4, B7-H2, Gi24/VISTA/B7-H5, B7-H3, ICOS, B7-H4, PD-1, B7-H6, PD-L2/B7-DC, B7-H7, PDCD6, LILRA3/CD85e, LILRB2/CD85d/ILT4, LILRA4/CD85g/ILT7, LILRB3/CD85a/ILT5, LILRB1/CD85j/ILT2, LILRB4/CD85k/ILT3, 4-1BB/TNFRSF9/CD137, GITR Ligand/TNFSF18, 4-1BB Ligand/TNFSF9, HVEM/TNFRSF14, BAFF/BLyS/TNFSF13B, LIGHT/TNFSF14, BAFF R/TNFRSF13C, Lymphotoxin-alpha/TNF-beta, CD27/TNFRSF7, OX40/TNFRSF4, CD27 Ligand/TNFSF7, OX40 Ligand/TNFSF4, CD30/TNFRSF8, RELT/TNFRSF19L, CD30 Ligand/TNFSF8, TACI/TNFRSF13B, CD40/TNFRSF5, TL1A/TNFSF15, CD40 Ligand/TNFSF5, TNF-alpha, DR3/TNFRSF25, TNF RII/TNFRSFIB, GITR/TNFRSF18, 2B4/CD244/SLAMF4, CD84/SLAMF5, BLAME/SLAMF8, CD229/SLAMF3, CD2, CRACC/SLAMF7, CD2F-10/SLAMF9, NTB-A/SLAMF6, CD48/SLAMF2, SLAM/CD150, CD58/LFA-3, CD7, DPPIV/CD26, CD96, EphB6, CD160, Integrin alpha 4 beta 1, CD200, Integrin alpha 4 beta 7/LPAM-1, CD300a/LMIR1, LAG-3, CRTAM, TIM-1/KIM-1/HAVCR, DAP12, TIM-4, Dectin-1/CLEC7A, TSLP R, ICOSL, and/or biological equivalents thereof.

The co-stimulatory molecule can be coupled to the nanoparticle in the same manner as the pMHC complex. In one embodiment of the present disclosure, the co-stimulatory molecule and the antigen/MHC complex are separately attached to the nanoparticle. In another embodiment of the disclosure, the co-stimulatory molecule and the pMHC complex are first complexed together and are then subsequently complexed to the nanoparticle. Multiple co-stimulatory molecules may be coupled to the nanoparticle; these may be multiple of the same co-stimulatory molecule or multiple different co-stimulatory molecules. Typically, polypeptide complexes are added to the nanoparticles to yield nanoparticles with adsorbed or coupled polypeptide complexes having a ratio of number of co-stimulatory molecules:number of nanoparticles from about 1 to 6000 molecules per nanoparticle, or alternatively at least about or at most about 0.1, 0.5, 1, 10, 100, 500, 1000, 2000, 3000, 4000, 5000, 6000 or more to: 1, and ranges in between, typically between about 0.1:1 to about 50:1. In another aspect, the ratio of the co-stimulatory molecule to the pMHC complex can be from about 0.1, 0.5, 1, 2, 5, 10, 50 or more to 1, preferably a ratio of 1:1, 1:2, 1:9, 1:10, 1:100, 2:1, 9:1, 10:1, or 100:1 of co-stimulatory molecule:pMHC complex is obtained. Similarly, density of the co-stimulatory molecules relative to nanoparticle surface area may be calculated according to the same relative formula as the pMHC complexes. In certain embodiments, the density of the co-stimulatory molecule per unit surface area of the nanoparticle is between about 0.0022 co-stimulatory molecules/100 nm² to about 13.26 co-stimulatory molecules/100 nm². In some embodiments, the density range of the co-stimulatory molecules may be the same or different from the density range for the pMHC complexes.

In some embodiments, wherein the nanoparticle comprises a one or more co-stimulatory molecules and does not comprise a pMHC complex, the nanoparticle has a co-stimulatory density of about 0.2 co-stimulatory molecule/100 nm² to about 6.5 co-stimulatory molecule/100 nm², or from about 0.2 co-stimulatory molecule/100 nm² to about 6 co-stimulatory molecule/100 nm², or from about 0.2 co-stimulatory molecule/100 nm² to about 5.8 co-stimulatory molecule/100 nm², or from about 0.2 co-stimulatory molecule/100 nm² to about 5.75 co-stimulatory molecule/100 nm², or from about 0.2 co-stimulatory molecule/100 nm² to about 5.5 co-stimulatory molecule/100 nm², or from about 0.2 co-stimulatory molecule/100 nm² to about 5 co-stimulatory molecule/100 nm², or from about 0.2 co-stimulatory molecule/100 nm² to about 4.5 co-stimulatory molecule/100 nm², or from about 0.2 co-stimulatory molecule/100 nm² to about 4 co-stimulatory molecule/100 nm², or from about 0.2 co-stimulatory molecule/100 nm² to about 3.5 co-stimulatory molecule/100 nm², or from about 0.2 co-stimulatory molecule/100 nm² to about 3 co-stimulatory molecule/100 nm², or from about 0.2 co-stimulatory molecule/100 nm² to about 2.5 co-stimulatory molecule/100 nm², or from about 0.2 co-stimulatory molecule/100 nm² to about 2 co-stimulatory molecule/100 nm², or from about 0.2 co-stimulatory molecule/100 nm² to about 1.5 co-stimulatory molecule/100 nm², or from about 0.2 co-stimulatory molecule/100 nm² to about 1.25 co-stimulatory molecule/100 nm², or from about 0.2 co-stimulatory molecule/100 nm² to about 1 co-stimulatory molecule/100 nm², or from about 0.2 co-stimulatory molecule/100 nm² to about 0.75 co-stimulatory molecule/100 nm².

In another aspect, the nanoparticle may have a co-stimulatory molecule density of from about 0.11 co-stimulatory molecule/100 nm² to about 5 co-stimulatory molecule/100 nm², or from about 0.11 co-stimulatory molecule/100 nm² to about 4.5 co-stimulatory molecule/100 nm², or from about 0.11 co-stimulatory molecule/100 nm² to about 4 co-stimulatory molecule/100 nm², or from about 0.11 co-stimulatory molecule/100 nm² to about 3.5 co-stimulatory molecule/100 nm², or from about 0.11 co-stimulatory molecule/100 nm² to about 3 co-stimulatory molecule/100 nm², or from about about 0.11 co-stimulatory molecule/100 nm² to about 2.5 co-stimulatory molecule/100 nm², or from about 0.11 co-stimulatory molecule/100 nm² to about 2 co-stimulatory molecule/100 nm², or from about 0.11 co-stimulatory molecule/100 nm² to about 1.5 co-stimulatory molecule/100 nm², or from about 0.11 co-stimulatory molecule/100 nm² to about 1 pMHC/100 nm², or from about 0.11 co-stimulatory molecule/100 nm² to about 0.75 co-stimulatory molecule/100 nm². In some aspects, the nanoparticle core has a co-stimulatory molecule density of from about 0.11 co-stimulatory molecule/100 nm² to about 5 co-stimulatory molecule/100 nm², or 0.12 co-stimulatory molecule/100 nm² to about 4.5 co-stimulatory molecule/100 nm², or from about 0.13 co-stimulatory molecule/100 nm² to about 4 co-stimulatory molecule/100 nm², or from about 0.14 co-stimulatory molecule/100 nm² to about 3.5 co-stimulatory molecule/100 nm², or from about 0.12 co-stimulatory molecule/100 nm² to about 2 co-stimulatory molecule/100 nm², or from about 0.25 co-stimulatory molecule/100 nm² to about 1.5 co-stimulatory molecule/100 nm², or from about 0.3 co-stimulatory molecule/100 nm² to about 0.75 co-stimulatory molecule/100 nm². In a further aspect, the nanoparticle core has a co-stimulatory molecule density of from about 0.2 co-stimulatory molecule/100 nm² to about 0.65 co-stimulatory molecule/100 nm², or alternatively from about 0.25 co-stimulatory molecule/100 nm² to about 0.45 co-stimulatory molecule/100 nm², or alternatively from about 0.3 co-stimulatory molecule/100 nm² to about 0.4 co-stimulatory molecule/100 nm².

In some embodiments, wherein the nanoparticle comprises a pMHC complex and one or more co-stimulatory molecules, the nanoparticle has a co-stimulatory density of about 0.4 co-stimulatory molecule/100 nm² to about 13 co-stimulatory molecule/100 nm², or from about 0.4 co-stimulatory molecule/100 nm² to about 12 co-stimulatory molecule/100 nm², or from about 0.4 co-stimulatory molecule/100 nm² to about 11.6 co-stimulatory molecule/100 nm², or from about 0.4 co-stimulatory molecule/100 nm² to about 11.5 co-stimulatory molecule/100 nm², or from about 0.4 co-stimulatory molecule/100 nm² to about 11 co-stimulatory molecule/100 nm², or from about 0.4 co-stimulatory molecule/100 nm² to about 10 co-stimulatory molecule/100 nm², or from about 0.4 co-stimulatory molecule/100 nm² to about 9 co-stimulatory molecule/100 nm², or from about 0.4 co-stimulatory molecule/100 nm² to about 8 co-stimulatory molecule/100 nm², or from about 0.4 co-stimulatory molecule/100 nm² to about 7 co-stimulatory molecule/100 nm², or from about 0.4 co-stimulatory molecule/100 nm² to about 6 co-stimulatory molecule/100 nm², or from about 0.4 co-stimulatory molecule/100 nm² to about 5 co-stimulatory molecule/100 nm², or from about 0.4 co-stimulatory molecule/100 nm² to about 4 co-stimulatory molecule/100 nm², or from about 0.4 co-stimulatory molecule/100 nm² to about 3 co-stimulatory molecule/100 nm², or from about 0.4 co-stimulatory molecule/100 nm² to about 2.5 co-stimulatory molecule/100 nm², or from about 0.4 co-stimulatory molecule/100 nm² to about 2 co-stimulatory molecule/100 nm², or from about 0.4 co-stimulatory molecule/100 nm² to about 1.5 co-stimulatory molecule/100 nm².

In another aspect, the nanoparticle may have a co-stimulatory molecule density of from about 0.22 co-stimulatory molecule/100 nm² to about 10 co-stimulatory molecule/100 nm², or from about 0.22 co-stimulatory molecule/100 nm² to about 9 co-stimulatory molecule/100 nm², or from about 0.22 co-stimulatory molecule/100 nm² to about 8 co-stimulatory molecule/100 nm², or from about 0.22 co-stimulatory molecule/100 nm² to about 7 co-stimulatory molecule/100 nm², or from about 0.22 co-stimulatory molecule/100 nm² to about 6 co-stimulatory molecule/100 nm², or from about about 0.22 co-stimulatory molecule/100 nm² to about 5 co-stimulatory molecule/100 nm², or from about 0.22 co-stimulatory molecule/100 nm² to about 4 co-stimulatory molecule/100 nm², or from about 0.22 co-stimulatory molecule/100 nm² to about 3 co-stimulatory molecule/100 nm², or from about 0.22 co-stimulatory molecule/100 nm² to about 2 pMHC/100 nm², or from about 0.22 co-stimulatory molecule/100 nm² to about 1.5 co-stimulatory molecule/100 nm². In some aspects, the nanoparticle core has a co-stimulatory molecule density of from about 0.22 co-stimulatory molecule/100 nm² to about 10 co-stimulatory molecule/100 nm², or 0.24 co-stimulatory molecule/100 nm² to about 9 co-stimulatory molecule/100 nm², or from about 0.26 co-stimulatory molecule/100 nm² to about 8 co-stimulatory molecule/100 nm², or from about 0.28 co-stimulatory molecule/100 nm² to about 7 co-stimulatory molecule/100 nm², or from about 0.24 co-stimulatory molecule/100 nm² to about 4 co-stimulatory molecule/100 nm², or from about 0.5 co-stimulatory molecule/100 nm² to about 3 co-stimulatory molecule/100 nm², or from about 0.6 co-stimulatory molecule/100 nm² to about 1.5 co-stimulatory molecule/100 nm². In a further aspect, the nanoparticle has a co-stimulatory molecule density of from about 0.4 co-stimulatory molecule/100 nm² to about 1.3 co-stimulatory molecule/100 nm², or alternatively from about 0.5 co-stimulatory molecule/100 nm² to about 0.9 co-stimulatory molecule/100 nm², or alternatively from about 0.6 co-stimulatory molecule/100 nm² to about 0.8 co-stimulatory molecule/100 nm². Cytokines

In certain aspect, the NPs further comprise, or alternatively consist essentially of, or yet further consist of at least one cytokine molecule. As used herein, the term “cytokine” encompasses low molecular weight proteins secreted by various cells in the immune system that act as signaling molecules for regulating a broad range of biological processes within the body at the molecular and cellular levels. “Cytokines” include individual immunomodulating proteins that fall within the class of lymphokines, interleukins, or chemokines.

Non limiting examples are disclosed herein: for instance, IL-1A and IL-1B are two distinct members of the human interleukin-1 (IL-1) family. Mature IL-1A is a 18 kDa protein, also known as fibroblast-activating factor (FAF), lymphocyte-activating factor (LAF), B-cell-activating factor (BAF), leukocyte endogenous mediator (LEM), etc. IL-4 is a cytokine that induces T helper-2 (Th2) cell differentiation, and is closely related to and has similar functions to IL-13. IL-5 is produced by Th2 cells and mast cells. It acts to stimulate B cell growth and increase immunoglobulin secretion. It is also involved in eosinophil activation. IL-6 is an interleukin that can act as either a pro-inflammatory or anti-inflammatory cytokine. It is secreted by T cells and macrophages to stimulate immune response to trauma or other tissue damage leading to inflammation. IL-6 is also produced from muscle in response to muscle contraction. IL-8 is a chemokine produced by macrophages and other cell types such as epithelial cells and endothelial cells, and acts as an important mediator of the immune reaction in the innate immune system response. IL-12 is involved in the differentiation of naïve T cells to T helper (Th1 or Th2) cells. As a heterodimeric cytokine, IL-12 is formed after two subunits encoded by two separate genes, IL-12A (p35) and IL-12B (p40), dimerize following protein synthesis. IL-12p70 indicates this heterodimeric composition. IL-13, a cytokine secreted by many cell types, especially Th2 cells, is an important mediator of allergic inflammation and disease. IL-17 is a cytokine produced by T helper cells and is induced by IL-23, resulting in destructive tissue damage in delayed-type reactions. IL-17 functions as a pro-inflammatory cytokine that responds to the invasion of the immune system by extracellular pathogens and induces destruction of the pathogen's cellular matrix. IP-10, or Interferon gamma-induced protein 10, is also known as C-X-C motif chemokine 10 (CXCL10) or small-inducible cytokine B10. As a small cytokine belonging to the CXC chemokine family, IP-10 is secreted by several cell types (including monocytes, endothelial cells and fibroblasts) in response to IFN-γ. Macrophage Inflammatory Proteins (MIP) belong to the family of chemokines. There are two major forms of human MIP, MIP-1u and MIP-1(3, which are also known as chemokine (C-C motif) ligand 3 (CCL3) and CCL4, respectively. Both are produced by macrophages following stimulation with bacterial endotoxins. Granulocyte colony-stimulating factor (G-CSF or GCSF), also known as colony-stimulating factor 3 (CSF 3), is a colony-stimulating factor hormone. G-CSF is a glycoprotein, growth factor, and cytokine produced by a number of different tissues to stimulate the bone marrow to produce granulocytes and stem cells. G-CSF also stimulates the survival, proliferation, differentiation, and function of neutrophil precursors and mature neutrophils. Epidermal growth factor or EGF is a growth factor that plays an important role in the regulation of cell growth, proliferation, and differentiation by binding with high affinity to its receptor EGFR. Vascular endothelial growth factor (VEGF) is a family of growth factors that are important signaling proteins involved in both vasculogenesis (the de novo formation of the embryonic circulatory system) and angiogenesis (the growth of blood vessels from pre-existing vasculature).

The cytokine or cytokines can be coupled to the nanoparticle in the same manner as the pMHC complex. In one embodiment of the present disclosure, the cytokine or cytokines and the pMHC complex are separately attached to the nanoparticle. In another embodiment of the disclosure, the cytokine or cytokines molecule and the pMHC complex are first complexed together and are then subsequently complexed to the nanoparticle. Multiple cytokines may be coupled to the nanoparticle; these may be multiple of the same cytokine or different cytokines.

In some embodiments, the cytokine is complexed to an anti-cytokine antibody to form a cytokine/anti-cytokine antibody complex, which complex is subsequently complexed to the nanoparticle. In some embodiments, the cytokine/anti-cytokine antibody complex includes but is not limited to IL-2/anti-IL-2 complexes. The IL-2/anti-IL-2 complexes can have agonistic properties or antagonistic properties.

In some embodiments, the cytokine is complexed to a cytokine receptor to form a cytokine/cytokine receptor complex, which complex is subsequently complexed to the nanoparaticle. In some embodiments, the cytokine/cytokine receptor complex includes but is not limited to IL15/IL-15Ra and/or IL-1/IL-2Ra. In some embodiments, the IL15/IL-15Ra complex can function as a T-cell co-stimulator.

Typically, polypeptide complexes are added to the nanoparticles to yield nanoparticles with adsorbed or coupled polypeptide complexes having a ratio of number of cytokines:number of nanoparticles from about 1 to 5999 molecules per nanoparticle, or alternatively at least about or at most about 0.1, 0.5, 1, 10, 100, 500, 1000, 2000, 3000, 4000, 5000, 6000 or more to: 1, and ranges in between, for example between about 0.1:1 to about 50:1. In other aspects, the ratio of the cytokine to the antigen/MHC complex can be from about 0.1, 0.5, 1, 2, 5, 10, 50 or more to 1, preferably a ratio of 1:1, 1:2, 1:9, 1:10, 1:100, 2:1, 9:1, 10:1, or 100:1 of cytokine:antigen/MHC complex is obtained. Similarly, density of the cytokines relative to nanoparticle surface area may be calculated according to the same relative formula as the antigen/MHC complexes. In certain embodiments, the density of the cytokines per unit surface area of the nanoparticle is between about 0.0022 cytokines/100 nm² to about 13.26 cytokines/100 nm². In some embodiments, the density range of the cytokines may be the same or different from the density range for the antigen/MHC complexes.

### Antigenic Components

Certain aspects of the disclosure include methods and compositions concerning antigenic compositions including segments, fragments, or epitopes of polypeptides, peptides, nucleic acids, carbohydrates, lipids and other molecules that provoke or induce an antigenic response, generally referred to as antigens. In particular, autoantigens, or antigenic segments or fragments of such autoantigens, which lead to the destruction of a cell via an autoimmune response, can be identified and used in making a peptide-MHC/nanoparticle complex described herein.

Although specific examples of antigens and antigenic components are disclosed herein, the disclosure is not so limited. Unless specifically stated otherwise, included herein are equivalents of the isolated or purified polypeptide antigens, that comprise, or consist essentially of, or yet further consist of, the amino acid sequences as described herein, or a polypeptide having at least about 80% sequence identity, or alternatively at least 85%, or alternatively at least 90%, or alternatively at least 95%, or alternatively at least 98% sequence identity to the amino acid sequences of the antigens, or polypeptides encoded by polynucleotides having at about 80% sequence identity, or alternatively at least 85%, or alternatively at least 90%, or alternatively at least 95%, or alternatively at least 98% sequence identity to the polynucleotide encoding the amino acid sequences of the antigen, or its complement, or a polypeptide encoded by a polynucleotide that hybridizes under conditions of moderate to high stringency to a polynucleotide encoding the amino acid sequence of the antigens, or its complement. Also provided are isolated and purified polynucleotides encoding the antigen polypeptides disclosed herein, or amino acids having at least about 80% sequence identity thereto, or alternatively at least 85%, or alternatively at least 90%, or alternatively at least 95%, or alternatively at least 98% sequence identity to the disclosed sequences, or an equivalent, or a polynucleotide that hybridizes under stringent conditions to the polynucleotide, its equivalent or its complement and isolated or purified polypeptides encoded by these polynucleotides. The polypeptides and polynucleotides can be combined with non-naturally occurring substances with which they are not associated with in nature, e.g., carriers, pharmaceutically acceptable carriers, vectors and MHC molecules.

### Modified Peptides and Equivalents Thereto

The antigenic polypeptides, proteins and fragments thereof may be modified by various amino acid deletions, insertions, and/or substitutions. In particular embodiments, modified polypeptides and/or peptides are capable of modulating an immune response in a subject. As used herein, a “protein” or “polypeptide” or “peptide” refers to a molecule comprising at least five amino acid residues. In some embodiments, a wild-type version of a protein or peptide are employed, however, in many embodiments of the disclosure, a modified protein or polypeptide is employed to generate a peptide/MHC/nanoparticle complex. A peptide/MHC/nanoparticle complex can be used to generate an immune response and/or to modify the T cell population of the immune system (i.e., re-educate the immune system). The terms described above may be used interchangeably herein. A “modified protein” or “modified polypeptide” or “modified peptide” refers to a protein or polypeptide whose chemical structure, particularly its amino acid sequence, is altered with respect to the wild-type protein or polypeptide. In some embodiments, a modified protein or polypeptide or peptide has at least one modified activity or function (recognizing that proteins or polypeptides or peptides may have multiple activities or functions). It is specifically contemplated that a modified protein or polypeptide or peptide may be altered with respect to one activity or function yet retain a wild-type activity or function in other respects, such as immunogenicity or ability to interact with other cells of the immune system when in the context of an MHC/nanoparticle complex.

In certain embodiments, the size of a protein or polypeptide (wild-type or modified), including any complex of a protein or peptide of interest and in particular a MHC/peptide fusion, may comprise, but is not limited to 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino molecules or greater, including any range or value derivable therein, or derivative thereof. In certain aspects, 5, 6, 7, 8, 9, 10 or more contiguous amino acids, including derivatives thereof, and fragments of an autoantigen, such as those amino acid sequences disclosed and referenced herein, can be used as antigens. It is contemplated that polypeptides may be mutated by truncation, rendering them shorter than their corresponding wild-type form, but they might also be altered by fusing or conjugating a heterologous protein sequence with a particular function (e.g., for presentation as a protein complex, for enhanced immunogenicity, etc.).

As used herein, an “amino molecule” refers to any amino acid, amino acid derivative, or amino acid mimic known in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid.

Proteinaceous compositions may be made by any technique known to those of skill in the art, including (i) the expression of proteins, polypeptides, or peptides through standard molecular biological techniques, (ii) the isolation of proteinaceous compounds from natural sources, or (iii) the chemical synthesis of proteinaceous materials. The nucleotide as well as the protein, polypeptide, and peptide sequences for various genes have been previously disclosed, and may be found in the recognized computerized databases. One such database is the National Center for Biotechnology Information's GenBank and GenPept databases (on the World Wide Web at ncbi.nlm.nih.gov/). The all or part of the coding regions for these genes may be amplified and/or expressed using the techniques disclosed herein or as would be known to those of ordinary skill in the art.

Amino acid sequence variants of autoantigenic epitopes and other polypeptides of these compositions can be substitutional, insertional, or deletion variants. A modification in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more non-contiguous or contiguous amino acids of a peptide or polypeptide, as compared to wild-type. A peptide or polypeptide that results in an autoimmune response and in particular a pathologic autoimmune response are contemplated for use in methods of the disclosure.

Deletion variants typically lack one or more residues of the native or wild-type amino acid sequence. Individual residues can be deleted or a number of contiguous amino acids can be deleted. A stop codon may be introduced (by substitution or insertion) into an encoding nucleic acid sequence to generate a truncated protein. Insertional mutants typically involve the addition of material at a non-terminal point in the polypeptide. This may include the insertion of one or more residues. Terminal additions, called fusion proteins, may also be generated.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of a polypeptide or peptide is affected, such as avidity or affinity for a cellular receptor(s). Non-conservative changes typically involve substituting a residue with one that is chemically dissimilar, such as a polar or charged amino acid for a nonpolar or uncharged amino acid, and vice versa.

Proteins of the disclosure may be recombinant, or synthesized in vitro. Alternatively, a recombinant protein may be isolated from bacteria or other host cell.

The term “functionally equivalent codon” is used herein to refer to codons that encode the same amino acid, such as the six codons for arginine or serine, and also refers to codons that encode biologically equivalent amino acids (see Table 2).

It also will be understood that amino acid and nucleic acid sequences may include additional residues, such as additional N- or C-terminal amino acids, or 5′ or 3′ nucleic acid sequences, respectively, and yet still be essentially as set forth in one of the sequences disclosed herein, so long as the sequence meets the criteria set forth above, including the maintenance of biological protein activity (e.g., immunogenicity). The addition of terminal sequences particularly applies to nucleic acid sequences that may, for example, include various non-coding sequences flanking either of the 5′ or 3′ portions of the coding region.

### Disease-Relevant Antigens

The nanoparticles are useful in the therapeutic methods as described herein. The pMHC complex of the pMHC-NP is selected for use based on the disease to be treated. For example, a diabetes-relevant antigen is an antigen or fragment thereof that is expressed in the cell, tissue or organ targeted in that autoimmune disease and that is exposed to the immune system upon cell, tissue or organ damage caused by the autoimmune response, even if the antigen is not the trigger of the disease process or a key player in its pathogenesis, and when presented, produces an immune response that serves to treat diabetes; thus, a diabetes-relevant antigen meeting this definition is selected to treat diabetes. A MS-relevant antigen is selected to treat MS. A diabetes-relevant antigen would not be selected to treat MS. Non-limiting, exemplary disease-relevant antigens are disclosed herein and further, such antigens may be determined for a particular disease based on techniques, mechanisms, and methods well documented in the literature.

Non-limiting examples of diseases of interest include, but are not limited to, asthma, diabetes mellitus Type I and Type II, pre-diabetes, multiple sclerosis, peripheral neuropathy, allergic asthma, primary biliary cirrhosis, cirrhosis, Neuromyelitis optica spectrum disorder, Autoantibody-associated neurological syndromes such as Stiff Person syndrome, Autoimmune Encephalitis, Narcolepsy, Pemphigus vulgaris, Pemphigus foliaceous, Psoriasis, Sjogren's disease/syndrome, Inflammatory bowel disease (IBD), arthritis, Rheumatoid arthritis, Systemic Lupus Erythematosus (SLE), Scleroderma, ANCA-associated Vasculitis, Goodpasture Syndrome, Kawasaki's Disease, Celiac disease, autoimmune cardiomyopathy, idiopathic dilated cardiomyopathy (IDCM), Myasthyenia Gravis, Autoimmune Uveitis, Ankylosing Spondylitis, Grave's Disease, Immune Mediated Myopathies, anti-phospholipid syndrome (ANCA+), atherosclerosis, Autoimmune Hepatitis, Sclerosing Cholangitis, Primary Sclerosing Cholangitis, Dermatomyositis, Chronic Obstructive Pulmonary Disease, Spinal Cord Injury, traumatic injury, tobacco-induced lung destruction, emphysema, pemphigus, uveitis, any other relevant cancer and/or diseases of the central and peripheral nervous systems.

### Cancer/Tumor Relevant Antigens

In certain aspects, the disease-relevant antigen is a cancer relevant antigen. In further aspects, the cancer is carcinoma, sarcoma, myeloma, leukemia, lymphoma, and/or mixed types of metastases from these or other cancers. Exemplary cancer- or tumor-relevant antigens include but are not limited to those disclosed in the following Table 5.

TABLE 5
Lys Ile Ser Val Ser Leu Pro Leu Ser Leu Ser Gln Ser Val Cys
Gln Leu Ser Lys Asp Thr Ser Val Leu Thr Phe Thr Phe Cys
Cys Ser Asp Ala His Pro Gly Asp Ser Ser Gly Asp Ser Ser Gly Leu Asn
Arg Gly Glu Val Arg Gln Phe Thr Leu Arg His Trp Leu Lys Val
Gly Asp Tyr Leu Asn Asp Glu Ala Leu Trp Asn Lys Cys
Gly Lys Val Ile Asp Asp Asn Asp His Leu Ser Gln Glu Ile Cys
Leu Met Ala Asn Ser Thr Trp Gly Tyr Pro Phe His Asp Gly
Leu Asn Val Val Pro Trp Asn Leu Thr Leu Phe Ser Ile Leu
Thr His Ser Phe Thr Ala Phe Lys Arg His Val Cys
Asn Leu Ser Leu Pro Pro Ser Leu Ser Leu Ser Ile Cys
Glu Arg Pro Ser Ser Val Leu Thr Ile Tyr Asp Ile Gly Ile Gln Cys
Cys Tyr Gln Gln Tyr Thr Asn Leu Gln Glu Arg Pro Ser Ser Val
Thr Val Glu Pro Glu Thr Gly Asp Pro Val Thr Leu Arg Leu Cys
Cys Ser Arg Lys Lys Arg Ala Asp Lys Lys Glu Asn Gly Thr Lys Leu Leu
Phe Leu Leu Val Leu Gly Phe Ile Ile
Val Leu Pro Ser Val Ala Met Phe Leu
Leu Val Leu Gly Phe Ile Ile Ala Leu
Lys Val Val Thr Ser Ser Phe Val Val
Leu Val Pro Gly Thr Lys Phe Tyr Ile
Leu Leu Pro Ile Arg Thr Leu Pro Leu
Tyr Leu Val Lys Lys Gly Thr Ala Thr
Ser Leu Phe Ala Glu Thr Ile Trp Val
Met Leu Ile Ala Met Tyr Phe Tyr Thr
Leu Met Trp Thr Leu Pro Val Met Leu
Met Leu Ile Val Tyr Ile Phe Glu Cys
Tyr Ile Phe Glu Cys Ala Ser Cys Ile
Leu Val Leu Met Leu Ile Val Tyr Ile
Ala Leu Cys Arg Arg Arg Ser Met Val
Leu Leu Ser Gly Leu Ser Leu Phe Ala
Phe Leu Leu Val Val Gly Leu Ile Val
Leu Val Val Gly Leu Ile Val Ala Leu
Lys Val Val Lys Ser Asp Phe Val Val
Thr Leu Pro Val Gln Thr Leu Pro Leu
Asp Leu His Val Ile Ser Asn Asp Val
Val Leu Val His Pro Gln Trp Val Leu
Phe Leu Arg Pro Gly Asp Asp Ser Ser
Ala Leu Gly Thr Thr Cys Tyr Ala Ser
Lys Leu Gln Cys Val Asp Leu His Val
Glu Leu Ala His Tyr Asp Val Leu Leu
Asn Leu Asn Gly Ala Gly Asp Pro Leu
Thr Leu Arg Val Asp Cys Thr Pro Leu
Met Met Asn Asp Gln Leu Met Phe Leu
Ala Leu Phe Asp Ile Glu Ser Lys Val
Leu Leu His Glu Thr Asp Ser Ala Val
Val Leu Ala Lys Glu Leu Lys Phe Val
Ile Leu Leu Trp Gln Pro Ile Pro Val
Asp Leu Phe Gly Ile Trp Ser Lys Val
Pro Leu Glu Arg Phe Ala Glu Leu Val
Lys Gln Gly Asn Phe Asn Ala Trp Val
Asn Leu Leu Arg Arg Met Trp Val Thr
Asn Leu Phe Glu Thr Pro Ile Leu Ala
Asn Leu Phe Glu Thr Pro Val Glu Ala
Gly Leu Gln His Trp Val Pro Glu Leu
Val Gln Phe Val Ala Ser Tyr Lys Val
Arg Leu Leu Ala Ala Leu Cys Gly Ala
Leu Leu Leu Leu Thr Val Leu Thr Val
Leu Leu Leu Thr Val Leu Thr Val Val
Phe Leu Ser Phe His Ile Ser Asn Leu
Leu Leu Val Leu Val Cys Val Leu Val
Ala Leu Leu Val Leu Val Cys Val Leu
Ser Leu Ser Tyr Thr Asn Pro Ala Val
Asn Leu Thr Ile Ser Asp Val Ser Val
Ala Leu Ala Ser Thr Ala Pro Pro Val
Ala Ile Leu Cys Trp Thr Phe Trp Val
Phe Ile Leu Met Phe Ile Val Tyr Ala
Leu Thr Ala Glu Cys Ile Phe Phe Val
Met Leu Gln Asp Asn Cys Cys Gly Val
Ile Leu Cys Trp Thr Phe Trp Val Leu
Lys Ile Leu Leu Ala Tyr Phe Ile Leu
Phe Val Gly Ile Cys Leu Phe Cys Leu
Val Leu Leu Ser Val Ala Met Phe Leu
Leu Leu Ser Val Ala Met Phe Leu Leu
Ile Leu Gly Ser Leu Pro Phe Phe Leu
Ile Leu Asn Ala Tyr Leu Val Arg Val
Phe Leu Leu Val Gly Phe Ala Gly Ala
Asn Leu Gln Pro Gln Leu Ala Ser Val
Cys Met Phe Asp Ser Lys Glu Ala Leu
Tyr Leu Tyr Val Leu Val Asp Ser Ala
Tyr Met Asp Gly Thr Met Ser Gln Val
Lys Met Ala Arg Phe Ser Tyr Ser Val
Gly Leu Val Met Asp Glu His Leu Val
Phe Leu Pro Gly Cys Asp Gly Leu Val
Cys Met Leu Gly Ser Phe Cys Ala Cys
Tyr Leu Ala Phe Arg Asp Asp Ser Ile
Trp Leu Pro Lys Lys Cys Ser Leu Cys
Cys Leu Asn Gly Gly Thr Cys Met Leu
Met Leu Val Gly Ile Cys Leu Ser Ile
Phe Glu Leu Gly Leu Val Ala Gly Leu
Lys Met Val Arg Phe Ser Tyr Ser Val
Cys Leu Asn Glu Gly Thr Cys Met Leu
Met Leu Ala Gly Ile Cys Leu Ser Ile
Arg Leu Leu Phe Phe Leu Leu Phe Leu
Thr Leu Ala Tyr Leu Ile Phe Cys Leu
Leu Leu Phe Leu Thr Pro Met Glu Val
Lys Leu Met Ser Pro Lys Leu Tyr Val
Leu Leu Phe Phe Leu Leu Phe Leu Val
Ser Leu Phe Leu Gly Ile Leu Ser Val
Ala Ile Ser Gly Met Ile Leu Ser Ile
Phe Ile Arg Ala His Thr Pro Tyr Ile
Ser Leu Asn Phe Ile Arg Ala His Thr
Leu Lys Met Glu Ser Leu Asn Phe Ile
Ser His Phe Leu Lys Met Glu Ser Leu
Tyr Leu Phe Leu Gly Ile Leu Ser Val

Other cancer relevant antigens include those summarized in the Tables in this online database http://cancerimmunity.org/peptide/and incorporated herein by reference, last referenced May 6, 2015.

### Autoimmune-Disease Relevant Antigens

In certain aspects, the disease-relevant antigen comprised in the antigen-MHC complex is selected from an autoimmune disease-relevant antigen, an inflammation-relevant antigen, or an allergic disease-relevant antigen. In further aspects, the immune inflammation-relevant antigen is one or more selected from the group of an asthma-relevant antigen, a diabetes-relevant antigen, a pre-diabetes relevant antigen, a multiple sclerosis-relevant antigen, an allergic asthma-relevant antigen, a primary biliary cirrhosis-relevant antigen, a cirrhosis-relevant antigen, a Neuromyelitis optica spectrum disorder (Devic's disease, NMO)-relevant antigen, an autoimmune encephalitis-relevant antigen, an antigen relevant to autoantibody-mediated neurological syndromes, a Stiff Man syndrome-relevant antigen, a paraneoplastic disease-relevant antigen, antigens relevant to other diseases of the central and peripheral nervous systems, a Pemphigus vulgaris-relevant antigen, inflammatory bowel disease (IBD)-relevant antigen, Crohn's disease-relevant antigen, Ulcerative Colitis-relevant antigen, an arthritis-relevant antigen, a Rheumatoid Arthritis-relevant antigen, a systemic lupus erythematosus (SLE)-relevant antigen, a Celiac Disease relevant antigen, a psoriasis-relevant antigen, an Alopecia Areata-relevant antigen, an Acquired Thrombocytopenic Purpura-relevant antigen, an autoimmune cardiomyopathy-relevant antigen, an idiopathic dilated cardiomyopathy (IDCM)-relevant antigen, a Myasthyenia Gravis-relevant antigen, an Uveitis-relevant antigen, an Ankylosing Spondylitis-relevant antigen, a Grave's Disease-relevant antigen, a Hashimoto's thyroiditis-relevant antigen, an Immune Mediated Myopathies-relevant antigen, an anti-phospholipid syndrome (ANCA+)-relevant antigen, an atherosclerosis-relevant antigen, a scleroderma-relevant antigen, an autoimmune hepatitis-relevant antigen, a dermatomyositis-relevant antigen, a chronic obstructive pulmonary disease-relevant antigen, a spinal cord injury-relevant antigen, a traumatic injury-relevant antigen, a tobacco-induced lung destruction-relevant antigen, a Chronic Obstructive Pulmonary Disease (COPD)-relevant antigen, a lung emphysema-relevant antigen, a sclerosing cholangitis-relevant antigen, a peripheral neuropathy-relevant antigen, a narcolepsy-relevant antigen, a Goodpasture Syndrome-relevant antigen, a Kawasaki's Disease-relevant antigen, an autoimmune uveitis-relevant antigen, a colitis-relevant antigen, an emphysema-relevant antigen, a pemphigus-relevant antigen, a pemphigus folliaceus-relevant antigen, an arthritis-relevant antigen, a Sjogren's Syndrome-relevant antigen, an ANCA-associated vasculitis-relevant antigen, a primary sclerosing cholangitis-relevant antigen, an adipose tissue inflammation/diabetes type II-relevant antigen, or an obesity associated adipose tissue inflammation/insulin resistance-relevant antigen.

In certain aspects, the disease-relevant antigen is derived from one or more of the group: PPI, IGRP, GAD, peripherin, aGlia, PDC-E2, Insulin, DG1EC2, DG3, AQP4, PLP, MOG, MBP, CII, DERP1, DERP2, OVA, BacInt, CBir, Fla-X, Fla-2, YIDX, AChR, Thyroid peroxidase, Thyroid receptor, Phospholipid antigen, H4, H2B, H1, DNA, ApoB, ApoE, NMDAR, Voltage-gated potassium channel, Elastin, Arrestin, PERM_HUMAN Myeloperoxidase, PRTN3_HUMAN Myeloblastin, CP2D6_HUMAN Cytochrome P450 2D6, SPCS_HUMAN O-phosphoseryl-tRNA(Sec) selenium transferase, CAMP_HUMAN Cathelicidin antimicrobial peptide, DNA topoisomerase I, CENP-C, APOH_HUMAN Beta-2-glycoprotein 1, RO60_HUMAN 60 kDa SS-A/Ro ribonucleoprotein, LA_HUMAN Lupus La protein, IRBP, myosin, CD1d-binding lipid antigens, Cap18, CP2D6, SPCS, RO60, R052, LA, APOH, MPO, PRTN3, or HSP.

In some embodiments, the disease-relevant antigen is:
a) a diabetes-relevant antigen and is derived from an antigen selected from one or more of the group: preproinsulin (PPI), islet-specific glucose-6-phosphatase (IGRP), glutamate decarboxylase (GAD), islet cell autoantigen-2 (ICA2), insulin, proinsulin, or a fragment or an equivalent of each thereof;
b) a multiple sclerosis-relevant antigen and is derived from an antigen selected from one or more of the group: myelin basic protein, myelin associated glycoprotein, myelin oligodendrocyte protein, proteolipid protein, oligodendrocyte myelin oligoprotein, myelin associated oligodendrocyte basic protein, oligodendrocyte specific protein, heat shock proteins, oligodendrocyte specific proteins, NOGO A, glycoprotein Po, peripheral myelin protein 22, 2′3′-cyclic nucleotide 3′-phosphodiesterase, or a fragment or an equivalent of each thereof;
c) a Celiac Disease-relevant antigen and is derived from gliadin or a fragment or an equivalent thereof;
d) a primary biliary cirrhosis-relevant antigen and is derived from PDC-E2 or a fragment or an equivalent thereof;
e) a pemphigus folliaceus-relevant antigen and/or pemphigus vulgaris-relevant antigen and is derived from an antigen selected from one or more of the group: DG1, DG3, or a fragment or an equivalent of each thereof;
f) a neuromyelitis optica spectrum disorder-relevant antigen and is derived from AQP4 or a fragment or an equivalent thereof;
g) an arthritis-relevant antigen and is derived from an antigen selected from one or more of the group: heat shock proteins, immunoglobulin binding protein, heterogeneous nuclear RNPs, annexin V, calpastatin, type II collagen, glucose-6-phosphate isomerase, elongation factor human cartilage gp39, mannose binding lectin, citrullinated vimentin, type II collagen, fibrinogen, alpha enolase, anti-carbamylated protein (anti-CarP), peptidyl arginine deiminase type 4 (PAD4), BRAF, fibrinogen gamma chain, inter-alpha-trypsin inhibitor heavy chain H1, alpha-1-antitrypsin, plasma protease C1 inhibitor, gelsolin, alpha 1-B glycoprotein, ceruloplasmin, inter-alpha-trypsin inhibitor heavy chain H4, complement factor H, alpha 2 macroglobulin, serum amyloid, C-reactive protein, serum albumin, fibrogen beta chain, serotransferin, alpha 2 HS glycoprotein, vimentin, Complement C3, or a fragment or an equivalent of each thereof;
h) an allergic asthma-relevant antigen and is derived from an antigen selected from one or more of the group: DERP1, DERP2, or a fragment or an equivalent of each thereof;
i) an inflammatory bowel disease-relevant antigen and is derived from an antigen selected from one or more of the group: Flagelin, Fla-2, Fla-X, YIDX, bacteroides integrase, or a fragment or an equivalent of each thereof;
j) a systemic lupus erythematosus-relevant antigen and is derived from an antigen selected from one or more of the group: double-stranded (ds)DNA, ribonucleoprotein (RNP), Smith (Sm), Sjögren's-syndrome-related antigen A (SS-A)/Ro, Sjögren's-syndrome-related antigen B (SS-B)/La, RO60, RO52, histones, or a fragment or an equivalent of each thereof;
k) an atherosclerosis-relevant antigen and is derived from an antigen selected from one or more of the group: ApoB, ApoE or a fragment or an equivalent of each thereof;
l) a COPD-relevant antigen and/or emphysema-relevant antigen and is derived from elastin or a fragment or an equivalent thereof;
m) a psoriasis-relevant antigen and is derived from an antigen selected from one or more of the group: Cap18, ADMTSL5, ATL5, or a fragment or an equivalent of each thereof;
n) an autoimmune hepatitis-relevant antigen and is derived from an antigen selected from one or more of the group: CYP2D6, SLA, or a fragment or an equivalent of each thereof;
o) an uveitis-relevant antigen and is derived from arrestin or a fragment or an equivalent thereof;
p) a Sjogren's Syndrome-relevant antigen and is derived from an antigen selected from one or more of the group: (SS-A)/Ro, (SS-B)/La, MR3, RO60, RO52, or a fragment or an equivalent of each thereof;
q) a scleroderma-relevant antigen and is derived from an antigen selected from one or more of the group: CENP-C, TOP 1, RNA polymerase III, or a fragment or an equivalent of each thereof;
r) an anti-phospholipid syndrome-relevant antigen and is derived from APOH or a fragment or an equivalent thereof;
s) an ANCA-associated vasculitis-relevant antigen and is derived from an antigen selected from one or more of the group: MPO, PRTN3, or a fragment or an equivalent of each thereof; or
t) a Stiff Man Syndrome-relevant antigen and is derived from GAD or a fragment or an equivalent thereof.

### Diabetes-Relevant Antigens

Diabetes-relevant antigens include but are not limited to those derived from PPI, IGRP, GAD, islet cell autoantigen-2 (ICA2), and/or insulin. Autoreactive, diabetes-relevant antigenic peptides include, but are not limited to, include those listed in the following Table 6, in addition to the peptides and proteins disclosed in U.S. Publication 2005/0202032, which is incorporated herein by reference in its entirety, as well as equivalents and/or combinations of each thereof,

TABLE 6
Peptide

| | |
|---|---|
| hInsB₁₀₋₁₈ | HLVEALYLV |
| hIGRP₂₂₈₋₂₃₆ | LNIDLLWSV |
| hIGRP₂₆₅₋₂₇₃ | VLFGLGFAI |
| IGRP₂₀₆₋₂₁₄ | VYLKTNVFL |
| hIGRP₂₀₆₋₂₁₄ | VYLKTNLFL |
| NRP-A7 | KYNKANAFL |
| NRP-I4 | KYNIANVFL |
| NRP-V7 | KYNKANVFL |
| YAI/D^{b} | FQDENYLYL |
| INS B₁₅₋₂₃ | LYLVCGERG |
| PPI_{76-90 (K88S)} | SLQPLALEGSLQSRG |
| IGRP₁₃₋₂₅ | QHLQKDYRAYYTF |
| GAD₅₅₅₋₅₆₇ | NFFRMVISNPAAT |
| GAD_{555-567(557I)} | NFIRMVISNPAAT |
| IGRP₂₃₋₃₅ | YTFLNFMSNVGDP |
| B₂₄-C₃₆ | FFYTPKTRREAED |
| PPI₇₆₋₉₀ | SLQPLALEGSLQKRG |
| INS-I9 | LYLVCGERI |
| TUM | KYQAVTTTL |
| G6Pase | KYCLITIFL |
| Pro-insulin_{L2-10} | ALWMRLLPL |
| Pro-insulin_{L3-11} | LWMRLLPLL |
| Pro-insulin_{L6-14} | RLLPLLALL |
| Pro-insulin_{B5-14} | HLCGSHLVEA |
| Pro-insulin_{B10-18} | HLVEALYLV |
| Pro-insulin_{B14-22} | ALYLVCGER |
| Pro-insulin_{B15-24} | LYLVCGERGF |
| Pro-insulin_{B17-25} | LVCGERGFF |
| Pro-insulin_{B18-27} | VCGERGFFYT |
| Pro-insulin_{B20-27} | GERGFFYT |
| Pro-insulin_{B21-29} | ERGFFYTPK |
| Pro-insulin_{B25-C1} | FYTPKTRRE |
| Pro-insulin_{B27-C5} | TPKTRREAEDL |
| Pro-insulin_{C20-28} | SLQPLALEG |
| Pro-insulin_{C25-33} | ALEGSLQKR |
| Pro-insulin_{C29-A5} | SLQKRGIVEQ |
| Pro-insulin_{A1-10} | GIVEQCCTSI |
| Pro-insulin_{A2-10} | IVEQCCTSI |
| Pro-insulin_{A12-20} | SLYQLENYC |

### MS-Relevant Antigens

Antigens of the disclosure include antigens related to multiple sclerosis. Such antigens include, for example, those disclosed in U.S. Patent Application Publication No. 2012/0077686, and antigens derived from myelin basic protein, myelin associated glycoprotein, myelin oligodendrocyte protein, proteolipid protein, oligodendrocyte myelin oligoprotein, myelin associated oligodendrocyte basic protein, oligodendrocyte specific protein, heat shock proteins, oligodendrocyte specific proteins NOGO A, glycoprotein Po, peripheral myelin protein 22, or 2′3′-cyclic nucleotide 3′-phosphodiesterase. In certain embodiments, the antigen is derived from Myelin Oligodendrocyte Glycoprotein (MOG).

In still further aspects, peptide antigens for the treatment of MS and MS-related disorders include without limitation those listed in Table 7 as well as equivalents and/or combinations of each thereof:

TABLE 7
Peptide

| | |
|---|---|
| MOG₃₅₋₅₅ | MEVGWYRSPFSRVVHLYRNGK |
| MOG₃₆₋₅₅ | EVGWYRSPFSRVVHLYRNGK |
| MAG₂₈₇₋₂₉₅ | SLLLELEEV |
| MAG₅₀₉₋₅₁₇ | LMWAKIGPV |
| MAG₅₅₆₋₅₆₄ | VLFSSDFRI |
| MBP₁₁₀₋₁₁₈ | SLSRFSWGA |
| MOG₁₁₄₋₁₂₂ | KVEDPFYWV |
| MOG₁₆₆₋₁₇₅ | RTFDPHFLRV |
| MOG₁₇₂₋₁₈₀ | FLRVPCWKI |
| MOG₁₇₉₋₁₈₈ | KITLFVIVPV |
| MOG₁₈₈₋₁₉₆ | VLGPLVALI |
| MOG₁₈₁₋₁₈₉ | TLFVIVPVL |
| MOG₂₀₅₋₂₁₄ | RLAGQFLEEL |
| PLP₈₀₋₈₈ | FLYGALLLA |
| MAG₂₈₇₋₂₉₅ | SLLLELEEV |
| MAG₅₀₉₋₅₁₇ | LMWAKIGPV |
| MAG₅₅₆₋₅₆₄ | VLFSSDFRI |
| MOG₉₇₋₁₀₉ | TCFFRDHSYQEEA |
| MOG_{97-109(E107S)} | TCFFRDHSYQEEA |
| MOG_{97-109(E107S)} | TCFFRDHSYQSEA |
| MBP₈₉₋₁₀₁ | VHFFKNIVTPRTP |
| PLP₁₇₅₋₁₉₂ | YIYFNTWTTCQSIAFPSK |
| PLP₉₄₋₁₀₈ | GAVRQIFGDYKTTIC |
| MBP₈₆₋₉₈ | PVVHFFKNIVTPR |
| PLP₅₄₋₆₈ | NYQDYEYLINVIHAF |
| PLP₂₄₉₋₂₆₃ | ATLVSLLTFMIAATY |
| MOG₁₅₆₋₁₇₀ | LVLLAVLPVLLLQIT |
| MOG₂₀₁₋₂₁₅ | FLRVPCWKITLFVIV |
| MOG₃₈₋₅₂ | RHPIRALVGDEVELP |
| MOG₂₀₃₋₂₁₇ | RVPCWKITLFVIVPV |
| PLP₂₅₀₋₂₆₄ | TLVSLLTFMIAATYN |
| MPB₁₃₋₃₂ | KYLATASTMDHARHGFLPRH |
| MPB₈₃₋₉₉ | ENPVVHFFKNIVTPRTP |
| MPB₁₁₁₋₁₂₉ | LSRFSWGAEGQRPGFGYGG |
| MPB₁₄₆₋₁₇₀ | AQGTLSKIFKLGGRDSRSGSPMARR |
| MOG₂₂₃₋₂₃₇ | ALIICYNWLHRRLAG |
| MOG₆₋₂₀ | IGPRHPIRALVGDEV |
| PLP₈₈₋₁₀₂ | AEGFYTTGAVRQIFG |
| PLP₁₃₉₋₁₅₄ | HCLGKWLGHPDKFVGI |

### Celiac Disease (CD) Relevant Antigens

Antigens relevant to celiac disease include, but are not limited to, those derived from gliadin. In some embodiments, non-limiting types of gliadin include alpha/beta gliadin, γ-gliadin, or ω-gliadin. Other non-limiting exemplary celiac disease-relevant antigens include those listed in Table 8 as well as equivalents and/or combinations of each thereof. Table 8.

TABLE 8
Peptide

| | |
|---|---|
| aGlia₅₇₋₆₈ | QLQPFPQPELPY |
| aGlia₆₂₋₇₂ | PQPELPYPQPE |
| aGlia₂₁₇₋₂₂₉ | SGEGSFQPSQQNP |

### Primary Biliary Cirrhosis (PBC) Relevant Antigens

Antigens relevant to primary biliary cirrhosis include, but are not limited to, those derived from PDC-E2. Non-limiting examples of exemplary antigens include those listed in Table 9 as well as equivalents and/or combinations of each thereof.

TABLE 9
Peptide

| | |
|---|---|
| PDC-E2₁₂₂₋₁₃₅ | GDLIAEVETDKATV |
| PDC-E2₂₄₉₋₂₆₂ | GDLLAEIETDKATI |
| PDC-E2₂₄₉₋₂₆₃ | GDLLAEIETDKATIG |
| PDC-E2₆₂₉₋₆₄₃ | AQWLAEFRKYLEKPI |
| PDC-E2₇₂₋₈₆ | RLLLQLLGSPGRRYY |
| PDC-E2₃₅₃₋₃₆₇ | GRVFVSPLAKKLAVE |
| PDC-E2₄₂₂₋₄₃₆ | DIPISNIRRVIAQRL |
| PDC-E2₆₂₉₋₆₄₃ | AQWLAEFRKYLEKPI |
| PDC-E2₈₀₋₉₄ | SPGRRYYSLPPHQKV |
| PDC-E2₃₅₃₋₃₆₇ | GRVFVSPLAKKLAVE |
| PDC-E2₅₃₅₋₅₄₉ | ETIANDVVSLATKAR |

### Pemphigus Folliaceus (PF) and Pemphigus Vulgaris (PV) Relevant Antigens

Antigens relevant to PF and PV include, but are not limited to, those derived from desmoglein 3 (DG3) and/or desmoglein 1 (DG1). Non-limiting examples include those listed in Table 10 as well as equivalents and/or combinations of each thereof.

TABLE 10
Peptide

| | |
|---|---|
| DG1₂₁₆₋₂₂₉ | GEIRTMNNFLDREI |
| DG3₉₇₋₁₁₁ | FGIFVVDKNTGDINI |
| DG3₂₅₁₋₂₆₅ | CECNIKVKDVNDNFP |
| DG3₃₅₁₋₃₆₅ | NKAEFHQSVISRYRV |
| DG3₄₅₃₋₄₆₇ | DSTFIVNKTITAEVL |
| DG3₅₄₀₋₅₅₄ | SITTLNATSALLRAQ |
| DG3₂₈₀₋₂₉₄ | ILSSELLRFQVTDLD |
| DG3₃₂₆₋₃₄₀ | EGILKVVKALDYEQL |
| DG3₃₆₇₋₃₈₁ | STPVTIQVINVREGI |
| DG3₁₃₋₂₇ | AIFVVVILVHGELRI |
| DG3₃₂₃₋₃₃₇ | RTNEGILKVVKALDY |
| DG3₄₃₈₋₄₅₂ | DSKTAEIKFVKNMNR |
| DG1₄₈₋₆₂ | KREWIKFAAACREGE |
| DG1₂₀₆₋₂₂₂ | MFIINRNTGEIRTMN |
| DG1₃₆₃₋₃₇₇ | SQYKLKASAISVTVL |
| DG1₃₋₁₇ | WSFFRVVAMLFIFLV |
| DG1₁₉₂₋₂₀₆ | SKIAFKIIRQEPSDS |
| DG1₃₂₆₋₃₄₀ | TNVGILKVVKPLDYE |
| DG1₁₋₁₅ | MDWSFFRVVAMLFIF |
| DG1₃₅₋₄₉ | KNGTIKWHSIRRQKR |
| DG1₃₂₅₋₃₃₉ | RTNVGILKVVKPLDY |

### Neuromyelitis Optica Spectrum Disorder (NMO) Relevant Antigens

Antigens relevant to NMO include, but are not limited to, those derived from AQP4 or aquaporin 4. Non-limiting examples include those listed in Table 11 as well as equivalents and/or combinations of each thereof.

TABLE 11
Peptide

| | |
|---|---|
| AQP4₁₂₉₋₁₄₃ | GAGILYLVTPPSVVG |
| AQP4₂₈₄₋₂₉₈ | RSQVETDDLILKPGV |
| AQP4₆₃₋₇₆ | EKPLPVDMVLISLC |
| AQP4₁₂₉₋₁₄₃ | GAGILYLVTPPSVVG |
| AQP4₃₉₋₅₃ | TAEFLAMLIFVLLSL |

### Arthritis-Relevant Antigens

Antigens relevant to arthritis include, but are not limited to, those derived from heat shock proteins, immunoglobulin binding protein, heterogeneous nuclear RNPs, annexin V, calpastatin, type II collagen, glucose-6-phosphate isomerase, elongation factor human cartilage gp39, mannose binding lectin, citrullinated vimentin, type II collagen, fibrinogen, alpha enolase, anti-carbamylated protein (anti-CarP), peptidyl arginine deiminase type 4 (PAD4), BRAF, fibrinogen gamma chain, inter-alpha-trypsin inhibitor heavy chain H1, alpha-1-antitrypsin, plasma protease C1 inhibitor, gelsolin, alpha 1-B glycoprotein, ceruloplasmin, inter-alpha-trypsin inhibitor heavy chain H4, complement factor H, alpha 2 macroglobulin, serum amyloid, C-reactive protein, serum albumin, fibrogen beta chain, serotransferin, alpha 2 HS glycoprotein, vimentin, Complement C3, or a fragment or an equivalent of each thereof.

### Allergic Asthma Relevant Antigens

Antigens relevant to allergic asthma include, but are not limited to, those derived from DERP1 and DERP2. Non-limiting examples include those listed in Table 12 as well as equivalents and/or combinations of each thereof.

TABLE 12
Peptide

| | |
|---|---|
| DERP-1₁₆₋₃₀ | LRQMRTVTPIRMQGG |
| DERP-1₁₇₁₋₁₈₅ | AVNIVGYSNAQGVDY |
| DERP-1₁₁₀₋₁₂₄ | RFGISNYCQIYPPNV |
| DERP-2₂₆₋₄₀ | PCIIHRGKPFQLEAV |
| DERP-2₁₀₇₋₁₂₁ | TVKVMGDDGVLACAI |

### Inflammatory Bowel Disease-Relevant Antigens

Antigens relevant to inflammatory bowel disease include but are not limited to Crohn's Disease-relevant antigens and ulcerative colitis-relevant antigens. In some embodiments, inflammatory bowel disease-relevant antigens include, but are not limited to, those derived from bacteroides integrase, flagelin, flagellin 2 (Fla-2/Fla-X), or uncharacterized E. coli protein (YDX). Non-limiting examples include those listed in Table 13 as well as equivalents and/or combinations of each thereof.

TABLE 13
Peptide

| | |
|---|---|
| bacteroides integrase | EAINQGYMHADAYPF |
| antigen₁₈₃₋₁₉₇ | |
| bacteroides integrase | KDLTYTFLRDFEQYL |
| antigen₁₄₆₋₁₆₀ | |
| bacteroides integrase | RQLRTLVNEAINQGY |
| antigen₁₇₅₋₁₈₉ | |
| bacteroides integrase antigen₁₋₁₅ | MDKIRYRLVYNRQNT |
| bacteroides integrase | EAINQGYMHADAYPF |
| antigen₁₈₃₋₁₉₇ | |
| bacteroides integrase antigen₃₀₋₄₄ | LNQRKIYLKTNVYLK |
| bacteroides integrase antigen₇₀₋₈₄ | EYILYLQGIELGYWK |
| bacteroides integrase | TCATLLIHQGVAITT |
| antigen₃₃₇₋₃₅₁ | |
| bacteroides integrase | AKHMRQLRTLVNEAI |
| antigen₁₇₁₋₁₈₅ | |
| bacteroides integrase antigen₄₋₁₈ | IRYRLVYNRQNTLNR |
| bacteroides integrase | ENFIRINGKRWLYFK |
| antigen₂₅₆₋₂₇₀ | |
| Fla-2/Fla-X₃₆₆₋₃₈₀ | TGAAATYAIDSIADA |
| Fla-2/Fla-X₁₆₄₋₁₇₈ | NATFSMDQLKFGDTI |
| Fla-2/Fla-X₂₆₁₋₂₇₅ | DRTVVSSIGAYKLIQ |
| Fla-2/Fla-X₁₋₁₅ | MVVQHNLRAMNSNRM |
| Fla-2/Fla-X₅₁₋₆₅ | KMRKQIRGLSQASLN |
| Fla-2/Fla-X₂₆₉₋₂₈₃ | GAYKLIQKELGLASS |
| Fla-2/Fla-X₄₋₁₈ | QHNLRAMNSNRMLGI |
| Fla-2/Fla-X₂₇₁₋₂₈₅ | YKLIQKELGLASSIG |
| YIDX₇₈₋₉₂ | ADDIVKMLNDPALNR |
| YIDX₉₃₋₁₀₇ | HNIQVADDARFVLNA |
| YIDX₉₈₋₁₁₂ | ADDARFVLNAGKKKF |
| YIDX₂₃₋₃₇ | GCISYALVSHTAKGS |
| YIDX₇₈₋₉₂ | ADDIVKMLNDPALNR |
| YIDX₁₉₅₋₂₀₉ | LPVTVTLDIITAPLQ |
| YIDX₂₂₋₃₆ | SGCISYALVSHTAKG |
| YIDX₈₀₋₉₄ | DIVKMLNDPALNRHN |
| YIDX₁₀₁₋₁₁₅ | ARFVLNAGKKKFTGT |

### Systemic Lupus Erythematosus (SLE) Relevant Antigens

Antigens relevant to SLE include, but are not limited to, those derived from H4, H2B, H1′, dsDNA, RNP, Smith (Sm), Sjogren's Syndrome-related Antigen A (SS-A)/Ro, Sjogren's Syndrome-related Antigen B (SS-B)/La, and/or histones. In some embodiments, SS-A includes but is not limited to RO60 and RO52. In some embodiments, histones includes but are not limited to H4, H2B, H1′. Non-limiting examples include those listed in Table 14 as well as equivalents and/or combinations of each thereof.

TABLE 14
Peptide

| | |
|---|---|
| H4₇₁₋₉₄ | TYTEHAKRKTVTAMDVVYALKRQG |
| H4₇₄₋₈₈ | EHAKRKTVTAMDVVY |
| H4₇₆₋₉₀ | AKRKTVTAMDVVYAL |
| H4₇₅₋₈₉ | HAKRKTVTAMDVVYA |
| H4₇₈₋₉₂ | RKTVTAMDVVYALKR |
| H4₈₀₋₉₄ | TVTAMDVVYALKRQ |
| H2B₁₀₋₂₄ | PKKGSKKAVTKAQKK |
| H2B₁₆₋₃₀ | KAVTKAQKKDGKKRK |
| H1′₂₂₋₄₂ | STDHPKYSDMIVAAIQAEKNR |
| H1′₂₇₋₄₁ | KYSDMIVAAIQAEKN |

### Atherosclerosis Relevant Antigens

Antigens relevant to atherosclerosis include, but are not limited to, those derived from Apolipoprotein B (ApoB) or Apolipoprotein E (ApoE). Non-limiting examples include those listed in Table 15 as well as equivalents and/or combinations of each thereof.

TABLE 15
Peptide

| | |
|---|---|
| ApoB₃₅₀₁₋₃₅₁₆ | SQEYSGSVANEANVY |
| ApoB₁₉₅₂₋₁₉₆₆ | SHSLPYESSISTALE |
| ApoB₉₇₈₋₉₉₃ | TGAYSNASSTESASY |
| ApoB₃₄₉₈₋₃₅₁₃ | SFLSQEYSGSVANEA |
| ApoB_{210A} | KTTKQSFDLSVKAQYKKNKH |
| ApoB_{210B} | KTTKQSFDLSVKAQY |
| ApoB_{210C} | TTKQSFDLSVKAQYK |

Chronic Obstructive Pulmonary Disease (COPD) and/or Emphysema Relevant Antigens

Antigens relevant to COPD and/or emphysema include, but are not limited to, those derived from elastin. Non-limiting examples include those listed in Table 16 as well as equivalents and/or combinations of each thereof.

TABLE 16
Peptide

| | |
|---|---|
| elastin₈₉₋₁₀₃ | GALVPGGVADAAAAY |
| elastin₆₉₈₋₇₁₂ | AAQFGLVGAAGLGGL |
| elastin₈₋₂₂ | APRPGVLLLLLSILH |
| elastin₉₄₋₁₀₈ | GGVADAAAAYKAAKA |
| elastin₁₃₋₂₇ | VLLLLLSILHPSRPG |
| elastin₆₉₅₋₇₀₉ | AAKAAQFGLVGAAGL |
| elastin₅₆₃₋₅₇₇ | VAAKAQLRAAAGLGA |
| elastin₅₅₈₋₅₇₂ | KSAAKVAAKAQLRAA |
| elastin₆₉₈₋₇₁₂ | AAQFGLVGAAGLGGL |
| elastin₅₆₆₋₅₈₀ | KAQLRAAAGLGAGIP |
| elastin₆₄₅₋₆₅₉ | VPGALAAAKAAKYGA |

### Psoriasis-Relevant Antigens

Antigens relevant to psoriasis include but are not limited to those listed in the following Table 17, as well as equivalents and/or combinations thereof. Other non-limiting exemplary psoriasis-relevant antigens can be derived from human adamis-like protein 5 (ATL5), cathelicidin antimicrobial peptide (CAP18), and/or ADAMTS-like protein 5 (ADMTSL5).

TABLE 17
Peptide

| | |
|---|---|
| Cap18₆₄₋₇₈ | RPTMDGDPDTPKPVS |
| Cap18₃₄₋₄₈ | SYKEAVLRAIDGINQ |
| Cap18₄₇₋₆₁ | NQRSSDANLYRLLDL |
| Cap18₁₅₁₋₁₆₅ | KRIVQRIKDFLRNLV |
| Cap18₁₄₉₋₁₆₃ | EFKRIVQRIKDFLRN |
| Cap18₁₅₂₋₁₆₆ | RIVQRIKDFLRNLVP |
| Cap18₁₃₁₋₁₄₅ | RFALLGDFFRKSKEK |
| Cap18₂₄₋₃₈ | QRIKDFLRNLVPRTE |
| ADMTSL5₂₄₅₋₂₅₉ | DGRYVLNGHWVVSPP |
| ADMTSL5₂₆₇₋₂₈₁ | THVVYTRDTGPQETL |
| ADMTSL5₃₇₂₋₃₈₆ | RLLHYCGSDFVFQAR |
| ADMTSL5₂₈₉₋₃₀₃ | HDLLLQVLLQEPNPG |
| ADMTSL5₃₉₆₋₄₁₀ | ETRYEVRIQLVYKNR |
| ADMTSL5₄₃₃₋₄₄₇ | HRDYLMAVQRLVSPD |
| ADMTSL5₁₄₂₋₁₅₆ | EGHAFYHSFGRVLDG |
| ADMTSL5₂₃₆₋₂₅₀ | RNHLALMGGDGRYVL |
| ADMTSL5₃₀₁₋₃₁₅ | NPGIEFEFWLPRERY |
| ADMTSL5₂₀₃₋₂₁₇ | VQRVFRDAGAFAGYW |
| ADMTSL5₄₀₄₋₄₁₈ | QLVYKNRSPLRAREY |

### Autoimmune Hepatitis-Relevant Antigens

Autoimmune hepatitis-relevant antigens include but are not limited to those disclosed in the following Table 18, as well as equivalents and/or combinations thereof. Other non-limiting exemplary autoimmune hepatitis-relevant antigens can be derived from microsomal cytochrome P450IID6 (CYP2D6) and/or soluble liver antigen (SLA).

TABLE 18
Peptide

| | |
|---|---|
| CYP2D6₁₉₃₋₂₀₇ | RRFEYDDPRFLRLLD |
| CYP2D6₇₆₋₉₀ | TPVVVLNGLAAVREA |
| CYP2D6₂₉₃₋₃₀₇ | ENLRIVVADLFSAGM |
| CYP2D6₃₁₃₋₃₃₂ | TLAWGLLLMILHPDVQRRVQ |
| CYP2D6₃₉₃₋₄₁₂ | TTLITNLSSVLKDEAVWEKP |
| CYP2D6₁₉₉₋₂₁₃ | DPRFLRLLDLAQEGL |
| CYP2D6₄₅₀₋₄₆₄ | RMELFLFFTSLLQHF |
| CYP2D6₃₀₁₋₃₁₅ | DLFSAGMVTTSTTLA |
| CYP2D6₄₅₂₋₄₆₆ | ELFLFFTSLLQHFSF |
| CYP2D6₅₉₋₇₃ | DQLRRRFGDVFSLQL |
| CYP2D6₁₃₀₋₁₄₄ | EQRRFSVSTLRNLGL |
| CYP2D6₁₉₃₋₂₁₂ | RRFEYDDPRFLRLLDLAQEG |
| CYP2D6₃₀₅₋₃₂₄ | AGMVTTSTTLAWGLLLMILH |
| CYP2D6₁₃₁₋₁₄₅ | QRRFSVSTLRNLGLG |
| CYP2D6₂₁₆₋₂₃₀ | ESGFLREVLNAVPVL |
| CYP2D6₂₃₈₋₂₅₂ | GKVLRFQKAFLTQLD |
| CYP2D6₁₉₉₋₂₁₃ | DPRFLRLLDLAQEGL |
| CYP2D6₂₃₅₋₂₅₂ | GKVLRFQKAFLTQLD |
| CYP2D6₂₉₃₋₃₀₇ | ENLRIVVADLFSAGM |
| CYP2D6₃₈₁₋₃₉₅ | DIEVQGFRIPKGTTL |
| CYP2D6₄₂₉₋₄₄₃ | KPEAFLPFSAGRRAC |
| SLA₃₃₄₋₃₄₈ | YKKLLKERKEMFSYL |
| SLA₁₉₆₋₂₁₀ | DELRTDLKAVEAKVQ |
| SLA₁₁₅₋₁₂₉ | NKITNSLVLDIIKLA |
| SLA₃₇₃₋₃₈₆ | NRLDRCLKAVRKER |
| SLA₁₈₆₋₁₉₇ | LIQQGARVGRID |
| SLA₃₁₇₋₃₃₁ | SPSLDVLITLLSLGS |
| SLA₁₇₁₋₁₈₅ | DQKSCFKSMITAGFE |
| SLA₄₁₇₋₄₃₁ | YTFRGFMSHTNNYPC |
| SLA₃₅₉₋₃₇₃ | YNERLLHTPHNPISL |
| SLA₂₁₅₋₂₂₉ | DCILCIHSTTSCFAP |
| SLA₁₁₁₋₁₂₅ | SSLLNKITNSLVLDI |
| SLA₁₁₀₋₁₂₄ | GSSLLNKITNSLVLD |
| SLA₂₉₉₋₃₁₃ | NDSFIQEISKMYPGR |
| SLA₃₄₂₋₃₅₆ | KEMFSYLSNQIKKLS |
| SLA₄₉₋₆₃ | STLELFLHELAIMDS |
| SLA₁₁₉₋₁₃₃ | NSLVLDIIKLAGVHT |
| SLA₂₆₀₋₂₇₄ | SKCMHLIQQGARVGR |
| SLA₂₆₋₄₀ | RSHEHLIRLLLEKGK |
| SLA₈₆₋₁₀₀ | RRHYRFIHGIGRSGD |
| SLA₃₃₁₋₃₄₅ | SNGYKKLLKERKEMF |

### Uveitis-Relevant Antigens

Uveitis-relevant antigens include but are not limited to those disclosed in the following Table 19, as well as equivalents and/or combinations thereof. Other non-limiting exemplary uveitis-relevant antigens can be derived from arrestin, human retinal S-antigen, and/or interphotoreceptor retinoid-binding protein (IRBP).

TABLE 19
Peptide

| | |
|---|---|
| arrestin₁₉₉₋₂₁₃ | QFFMSDKPLHLAVSLN |
| arrestin₇₇₋₉₁ | DVIGLTFRRDLYFSR |
| arrestin₂₅₀₋₂₆₄ | NVVLYSSDYYVKPVA |
| arrestin₁₇₂₋₁₈₆ | SSVRLLIRKVQHAPL |
| arrestin₃₅₄₋₃₆₈ | EVPFRLMHPQPEDPA |
| arrestin₂₃₉₋₂₅₃ | KKIKAFVEQVANVVL |
| arrestin₁₀₂₋₁₁₆ | STPTKLQESLLKKLG |
| arrestin₅₉₋₇₃ | KKVYVTLTCAFRYGQ |
| arrestin₂₈₀₋₂₉₄ | KTLTLLPLLANNRER |
| arrestin₂₉₁₋₃₀₆ | NRERRGIALDGKIKHE |
| arrestin₁₉₅₋₂₀₉ | EAAWQFFMSDKPLHL |
| arrestin₂₀₀₋₂₁₄ | QFFMSDKPLHLAVSL |

### Sjogren's Syndrome-Relevant Antigens

Sjogren's Syndrome-relevant antigens include but are not limited to those disclosed in the following Table 20, as well as equivalents and/or combinations thereof. Other non-limiting exemplary Sjogren's Syndrome-relevant antigens can be derived from (SS-A)/Ro, (SS-B)/La, RO60, RO52, and/or muscarinic receptor 3 (MR3).

TABLE 20
Peptide

| | |
|---|---|
| RO60₁₂₇₋₁₄₁ | TFIQFKKDLKESMKC |
| RO60₅₂₃₋₅₃₇ | DTGALDVIRNFTLDM |
| RO60₂₄₃₋₂₅₇ | EVIHLIEEHRLVREH |
| RO60₄₈₄₋₄₉₈ | REYRKKMDIPAKLIV |
| RO60₃₄₇₋₃₆₁ | EEILKALDAAFYKTF |
| RO60₃₆₉₋₃₈₃ | KRFLLAVDVSASMNQ |
| RO60₄₂₆₋₄₄₀ | TDMTLQQVLMAMSQI |
| RO60₂₆₇₋₂₈₁ | EVWKALLQEMPLTAL |
| RO60₁₇₈₋₁₉₂ | SHKDLLRLSHLKPSS |
| RO60₃₅₈₋₃₇₂ | YKTFKTVEPTGKRFL |
| RO60₂₂₁₋₂₃₅ | ETEKLLKYLEAVEKV |
| RO60₃₁₈₋₃₃₂ | RIHPFHILIALETYK |
| RO60₄₀₇₋₄₂₁ | EKDSYVVAFSDEMVP |
| RO60₄₅₉₋₄₇₃ | TPADVFIVFTDNETF |
| RO60₅₁₋₆₅ | QKLGLENAEALIRLI |
| RO60₃₁₂₋₃₂₆ | KLLKKARIHPFHILI |
| LA₂₄₁₋₂₅₅ | DDQTCREDLHILFSN |
| LA₁₀₁₋₁₁₅ | TDEYKNDVKNRSVYI |
| LA₁₅₃₋₁₆₇ | SIFVVFDSIESAKKF |
| LA₁₇₈₋₁₉₂ | TDLLILFKDDYFAKK |
| LA₁₉₋₃₃ | HQIEYYFGDFNLPRD |
| LA₃₇₋₅₁ | KEQIKLDEGWVPLEI |
| LA₁₃₃₋₁₄₇ | DKGQVLNIQMRRTLH |
| LA₅₀₋₆₄ | EIMIKFNRLNRLTTD |
| LA₃₂₋₄₆ | RDKFLKEQIKLDEGW |
| LA₁₅₃₋₁₆₇ | SIFVVFDSIESAKKF |
| LA₈₃₋₉₇ | SEDKTKIRRSPSKPL |
| LA₁₃₆₋₁₅₀ | QVLNIQMRRTLHKAF |
| LA₂₉₇₋₃₁₁ | RNKEVTWEVLEGEVE |
| LA₅₉₋₇₃ | NRLTTDFNVIVEALS |
| LA₁₅₁₋₁₆₅ | KGSIFVVFDSIESAK |
| LA₈₆₋₁₀₀ | KTKIRRSPSKPLPEV |
| LA₁₅₄₋₁₆₈ | IFVVFDSIESAKKFV |

### Scleroderma-Relevant Antigens

Scleroderma-relevant antigens include but are not limited to those disclosed in the following Table 21, as well as equivalents and/or combinations thereof. Non-limiting exemplary Scleroderma-relevant antigens can be derived from centromere autoantigen centromere protein C (CENP-C), DNA topoisomerase I (TOP1), and/or RNA polymerase III.

TABLE 21
Peptide

| | |
|---|---|
| TOP1₃₄₆₋₃₆₀ | KERIANFKIEPPGLF |
| TOP1₄₂₀₋₄₃₄ | QGSIKYIMLNPSSRI |
| TOP1₇₅₀₋₇₆₄ | QREKFAWAIDMADED |
| TOP1₄₁₉₋₄₃₃ | IQGSIKYIMLNPSSR |
| TOP1₅₉₁₋₆₀₅ | YNASITLQQQLKELT |
| TOP1₆₉₅₋₇₀₉ | EQLMKLEVQATDREE |
| TOP1₃₀₅₋₃₁₉ | SQYFKAQTEARKQMS |
| TOP1₃₄₆₋₃₆₀ | KERIANFKIEPPGLF |
| TOP1₄₁₉₋₄₃₃ | IQGSIKYIMLNPSSR |
| TOP1₄₂₅₋₄₃₉ | YIMLNPSSRIKGEKD |
| TOP1₆₁₄₋₆₂₈ | KILSYNRANRAVAIL |
| CENP-C₂₉₇₋₃₁₁ | KLIEDEFIIDESDQS |
| CENP-C₈₅₇₋₈₇₁ | KVYKTLDTPFFSTGK |
| CENP-C₈₈₇₋₉₀₁ | QDILVFYVNFGDLLC |
| CENP-C₂₁₂₋₂₂₆ | KVMLKKIEIDNKVSD |
| CENP-C₆₄₃₋₆₅₇ | EDNIMTAQNVPLKPQ |
| CENP-C₈₃₂₋₈₄₆ | TREIILMDLVRPQDT |
| CENP-C₁₆₇₋₁₈₁ | TSVSQNVIPSSAQKR |
| CENP-C₂₄₆₋₂₆₀ | RIRDSEYEIQRQAKK |
| CENP-C₈₄₆₋₈₆₀ | TYQFFVKHGELKVYK |
| CENP-C₁₄₉₋₁₆₃ | DEEFYLSVGSPSVLL |
| CENP-C₈₃₃₋₈₄₇ | REIILMDLVRPQDTY |
| CENP-C₈₄₇₋₈₆₁ | YQFFVKHGELKVYKT |

### Anti-Phospholipid Syndrome-Relevant Antigens

Anti-phospholipid syndrome relevant antigens include but are not limited to those disclosed in the following Table 22, as well as equivalents and/or combinations thereof. Non-limiting exemplary anti-phospholipid syndrome-relevant antigens can be derived from beta-2-glycoprotein 1 (BG2P1 or APOH).

TABLE 22
Peptide

| | |
|---|---|
| APOH₂₃₅₋₂₄₉ | HDGYSLDGPEEIECT |
| APOH₃₀₆₋₃₂₀ | KCSYTEDAQCIDGTI |
| APOH₂₃₇₋₂₅₁ | GYSLDGPEEIECTKL |
| APOH₂₉₅₋₃₀₉ | KVSFFCKNKEKKCSY |
| APOH₂₈₋₄₂ | DLPFSTVVPLKTFYE |
| APOH₁₇₃₋₁₈₇ | ECLPQHAMFGNDTIT |
| APOH₂₆₄₋₂₇₈ | CKVPVKKATVVYQGE |
| APOH₂₉₅₋₃₀₉ | KVSFFCKNKEKKCSY |
| APOH₄₉₋₆₃ | YSCKPGYVSRGGMRK |
| APOH₂₆₉₋₂₈₃ | KKATVVYQGERVKIQ |
| APOH₂₉₅₋₃₀₉ | KVSFFCKNKEKKCSY |
| APOH₃₂₁₋₃₅₅ | EVPKCFKEHSSLAFW |
| APOH₃₂₂₋₃₃₆ | VPKCFKEHSSLAFWK |
| APOH₃₂₄₋₃₃₈ | KCFKEHSSLAFWKTD |

### ANCA-Associated Vasculitis-Relevant Antigens

ANCA-associated vasculitis-relevant antigens include but are not limited to those disclosed in the following Table 23, as well as equivalents and/or combinations thereof. Non-limiting exemplary ANCA-associated vasculitis-relevant antigens can be derived from myeloperoxidase (MPO), proteinase (PRTN3), or bacterial permeability increasing factor (BPI).

TABLE 23
Peptide

| | |
|---|---|
| MPO₅₀₆₋₅₂₀ | QPFMFRLDNRYQPME |
| MPO₃₀₂₋₃₁₆ | RIKNQADCIPFFRSC |
| MPO₇₋₂₁ | SSLRCMVDLGPCWAG |
| MPO₆₈₉₋₇₀₃ | QQRQALAQISLPRII |
| MPO₂₄₈₋₂₆₂ | RSLMFMQWGQLLDHD |
| MPO₄₄₄₋₄₅₈ | QEARKIVGAMVQIIT |
| MPO₅₁₃₋₅₂₇ | DNRYQPMEPNPRVPL |
| MPO₉₇₋₁₁₁ | ELLSYFKQPVAATRT |
| MPO₆₁₆₋₆₃₀ | QLGTVLRNLKLARKL |
| MPO₄₆₂₋₄₇₆ | YLPLVLGPTAMRKYL |
| MPO₆₁₇₋₆₃₁ | LGTVLRNLKLARKLM |
| MPO₇₁₄₋₇₂₈ | KNNIFMSNSYPRDFV |
| PRTN3₄₄₋₅₈ | SLQMRGNPGSHFCGG |
| PRTN3₂₃₄₋₂₄₈ | TRVALYVDWIRSTLR |
| PRTN3₅₉₋₇₃ | TLIHPSFVLTAAHCL |
| PRTN3₁₁₇₋₁₃₁ | NDVLLIQLSSPANLS |
| PRTN3₁₆₄₋₁₇₈ | DPPAQVLQELNVTVV |
| PRTN3₇₁₋₈₅ | HCLRDIPQRLVNVVL |
| PRTN3₂₄₁₋₂₅₅ | DWIRSTLRRVEAKGR |
| PRTN3₅₉₋₇₃ | TLIHPSFVLTAAHCL |
| PRTN3₁₈₃₋₁₉₇ | RPHNICTFVPRRKAG |
| PRTN3₆₂₋₇₆ | HPSFVLTAAHCLRDI |
| PRTN3₁₁₈₋₁₃₂ | DVLLIQLSSPANLSA |
| PRTN3₂₃₉₋₂₅₃ | YVDWIRSTLRRVEAK |

### Stiff Man Syndrome-Relevant Antigens

Stiff Man Syndrome-relevant antigens include but are not limited to those disclosed in the following Table 24, as well as equivalents and/or combinations thereof. Non-limiting exemplary Stiff Man Syndrome-relevant antigens can be derived from glutamate decarboxylase (GAD). In some embodiments, GAD includes but is not limited to GAD65.

TABLE 24
Peptide

| | |
|---|---|
| GAD₂₁₂₋₂₂₆ | EYVTLKKMREIIGWP |
| GAD₅₅₅₋₅₆₉ | NFFRMVISNPAATHQ |
| GAD₂₉₇₋₃₁₁ | DSVILIKCDERGKMI |

It is contemplated that in compositions of the disclosure, there is between about 0.001 mg and about 10 mg of total protein per ml in the composition. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 50, 100 μg/ml or mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% may be peptide/MHC/nanoparticle complex.

The present disclosure contemplates the administration of a peptide/MHC/nanoparticle complex to effect a diagnosis, treatment or preventative therapy against the development of a disease or condition associated with autoimmune responses or cancer.

In addition, U.S. Pat. No. 4,554,101 (Hopp), which is incorporated herein by reference, teaches the identification and preparation of epitopes from primary amino acid sequences on the basis of hydrophilicity. Through the methods disclosed in Hopp, one of skill in the art would be able to identify potential epitopes from within an amino acid sequence and confirm their immunogenicity. Numerous scientific publications have also been devoted to the prediction of secondary structure and to the identification of epitopes, from analyses of amino acid sequences (Chou & Fasman, 1974a,b; 1978a,b; 1979). Any of these may be used, if desired, to supplement the teachings of Hopp in U.S. Pat. No. 4,554,101.

### Other Antigenic Components

Molecules other than peptides can be used as antigens or antigenic fragments in complex with MHC molecules. Such molecules include, but are not limited to, carbohydrates, lipids, small molecules, and the like. Carbohydrates are major components of the outer surface of a variety of cells. Certain carbohydrates are characteristic of different stages of differentiation and very often these carbohydrates are recognized by specific antibodies. Expression of distinct carbohydrates can be restricted to specific cell types. Autoantibody responses to endometrial and serum antigens have been shown to be a common feature of endometriosis. There has been described a serum autoantibody response in endometriosis to a number of previously identified antigens, including 2-Heremans Schmidt glycoprotein and carbonic anhydrase, which is specific for a carbohydrate epitope.

### Non-limiting, Exemplary Antigen-MHC Complexes

In certain embodiments, specific combinations of antigen and MHC may be optimized for the treatment of a specific disease. Non-limiting examples include, but are not limited to, the following examples:

For the treatment of type I diabetes, the antigen of the pMHC complex may be derived from an antigen of the group: PPI_{76-90(K88S)}, IGRP₁₃₋₂₅, GAD₅₅₅₋₅₆₇, GAD_{555-567(557I)}, IGRP₂₃₋₃₅, B₂₄-C₃₆, PPI₇₆₋₉₀, or a fragment or an equivalent of each thereof, and the MHC of the pMHC complex comprises all or part of a polypeptide of the group: HLA-DRB1*0401/DRA, HLA-DRB1*0301/DRA, or a fragment or an equivalent of each thereof.

In some embodiments, the antigen of the pMHC complex comprises a:
a) a diabetes-relevant antigen and is derived from an antigen selected from one or more of the group: preproinsulin (PPI), islet-specific glucose-6-phosphatase (IGRP), glutamate decarboxylase (GAD), islet cell autoantigen-2 (ICA2), insulin, proinsulin, or a fragment or an equivalent of each thereof;
b) a multiple sclerosis-relevant antigen and is derived from an antigen selected from one or more of the group: myelin basic protein, myelin associated glycoprotein, myelin oligodendrocyte protein, proteolipid protein, oligodendrocyte myelin oligoprotein, myelin associated oligodendrocyte basic protein, oligodendrocyte specific protein, heat shock proteins, oligodendrocyte specific proteins, NOGO A, glycoprotein Po, peripheral myelin protein 22, 2′3′-cyclic nucleotide 3′-phosphodiesterase, or a fragment or an equivalent of each thereof;
c) a Celiac Disease-relevant antigen and is derived from gliadin or a fragment or an equivalent thereof;
d) a primary biliary cirrhosis-relevant antigen and is derived from PDC-E2 or a fragment or an equivalent thereof;
e) a pemphigus folliaceus-relevant antigen and/or pemphigus vulgaris-relevant antigen and is derived from an antigen selected from one or more of the group: DG1, DG3, or a fragment or an equivalent of each thereof;
f) a neuromyelitis optica spectrum disorder-relevant antigen and is derived from AQP4 or a fragment or an equivalent thereof;
g) an arthritis-relevant antigen and is derived from an antigen selected from one or more of the group: heat shock proteins, immunoglobulin binding protein, heterogeneous nuclear RNPs, annexin V, calpastatin, type II collagen, glucose-6-phosphate isomerase, elongation factor human cartilage gp39, mannose binding lectin, citrullinated vimentin, type II collagen, fibrinogen, alpha enolase, anti-carbamylated protein (anti-CarP), peptidyl arginine deiminase type 4 (PAD4), BRAF, fibrinogen gamma chain, inter-alpha-trypsin inhibitor heavy chain H1, alpha-1-antitrypsin, plasma protease C1 inhibitor, gelsolin, alpha 1-B glycoprotein, ceruloplasmin, inter-alpha-trypsin inhibitor heavy chain H4, complement factor H, alpha 2 macroglobulin, serum amyloid, C-reactive protein, serum albumin, fibrogen beta chain, serotransferin, alpha 2 HS glycoprotein, vimentin, Complement C3, or a fragment or an equivalent of each thereof;
h) an allergic asthma-relevant antigen and is derived from an antigen selected from one or more of the group: DERP1, DERP2, or a fragment or an equivalent of each thereof;
i) an inflammatory bowel disease-relevant antigen and is derived from an antigen selected from one or more of the group: Flagelin, Fla-2, Fla-X, YIDX, bacteroides integrase, or a fragment or an equivalent of each thereof;
j) a systemic lupus erythematosus-relevant antigen and is derived from an antigen selected from one or more of the group: double-stranded (ds)DNA, ribonucleoprotein (RNP), Smith (Sm), Sjögren's-syndrome-related antigen A (SS-A)/Ro, Sjögren's-syndrome-related antigen B (SS-B)/La, RO60, RO52, histones, or a fragment or an equivalent of each thereof;
k) an atherosclerosis-relevant antigen and is derived from an antigen selected from one or more of the group: ApoB, ApoE or a fragment or an equivalent of each thereof;
l) a COPD-relevant antigen and/or emphysema-relevant antigen and is derived from elastin or a fragment or an equivalent thereof;
m) a psoriasis-relevant antigen and is derived from an antigen selected from one or more of the group: Cap18, ADMTSL5, ATL5, or a fragment or an equivalent of each thereof;
n) an autoimmune hepatitis-relevant antigen and is derived from an antigen selected from one or more of the group: CYP2D6, SLA, or a fragment or an equivalent of each thereof;
o) an uveitis-relevant antigen and is derived from arrestin or a fragment or an equivalent thereof;
p) a Sjogren's Syndrome-relevant antigen and is derived from an antigen selected from one or more of the group: (SS-A)/Ro, (SS-B)/La, MR3, RO60, RO52, or a fragment or an equivalent of each thereof;
q) a scleroderma-relevant antigen and is derived from an antigen selected from one or more of the group: CENP-C, TOP 1, RNA polymerase III, or a fragment or an equivalent of each thereof;
r) an anti-phospholipid syndrome-relevant antigen and is derived from APOH or a fragment or an equivalent thereof;
s) an ANCA-associated vasculitis-relevant antigen and is derived from an antigen selected from one or more of the group: MPO, PRTN3, or a fragment or an equivalent of each thereof; or
t) a Stiff Man Syndrome-relevant antigen and is derived from GAD or a fragment or an equivalent thereof.

In some embodiments, the MHC protein of the pMHC complex comprises all or part of a classical MHC class I protein, non-classical MHC class I protein, classical MHC class II protein, non-classical MHC class II protein, MHC dimers (Fc fusions), MHC tetramers, or a polymeric form of a MHC protein, wherein the MHC protein optionally comprises a knob-in-hole based MHC-alpha-Fc/MHC-beta-Fc heterodimer or multimer.

In some embodiments, the MHC protein of the pMHC complex comprises all or part of a polypeptide of the group: HLA DR, HLA DQ, HLA DP, HLA-A, HLA-B, HLA-C, HLA-E, HLA-F, HLA-G, CD1d, or a fragment or an equivalent of each thereof.

In some embodiments, the MHC protein of the pMHC complex comprises all or part of a polypeptide of the group: HLA-DR, HLA-DQ, HLA-DP, or a fragment or an equivalent of each thereof.

In some embodiments, the MHC protein of the pMHC complex comprises all or part of a polypeptide of the group: HLA-DRB1/DRA, HLA-DRB3/DRA, HLA-DRB4/DRA, HLA-DRB5/DRA, HLA-DQA1/HLA-DQB1, HLA-DPB1/HLA-DPA1, or a fragment or an equivalent of each thereof.

In certain aspects, the pMHC complex comprises:
a) a diabetes-relevant antigen derived from an antigen selected from one or more of the group: hInsB₁₀₋₁₈, hIGRP₂₂₈₋₂₃₆, hIGRP₂₆₅₋₂₇₃, IGRP₂₀₆₋₂₁₄, hIGRP₂₀₆₋₂₁₄, NRP-A7, NRP-I4, NRP-V7, YAI/D^{b}, INS B₁₅₋₂₃, PPI_{76-90 (K88S)}, IGRP₁₃₋₂₅, GAD₅₅₅₋₅₆₇, GAD_{555-567(557I)}, IGRP₂₃₋₃₅, B₂₄-C₃₆, PPI₇₆₋₉₀, INS-I9, TUM, G6Pase, Pro-insulin_{L2-10}, Pro-insulin_{L3-11}, Pro-insulin_{L6-14}, Pro-insulin_{B5-14}, Pro-insulin_{B10-18}, Pro-insulin_{B14-22}, Pro-insulin_{B15-24}, Pro-insulin_{B17-25}, Pro-insulin_{B18-27}, Pro-insulin_{B20-27}, Pro-insulin_{B21-29}, Pro-insulin_{B25-C1}, Pro-insulin_{B27-C5}, Pro-insulin_{C20-28}, Pro-insulin_{C25-33}, Pro-insulin_{C29-A5}, Pro-insulin_{A1-10}, Pro-insulin_{A2-10}, Pro-insulin_{A12-20}, or a fragment or an equivalent of each thereof;
b) a multiple sclerosis-relevant antigen derived from an antigen selected from one or more of the group: MOG₃₅₋₅₅, MOG₃₆₋₅₅, MAG₂₈₇₋₂₉₅, MAG₅₀₉₋₅₁₇, MAG₅₅₆₋₅₆₄, MBP₁₁₀₋₁₁₈, MOG₁₁₄₋₁₂₂, MOG₁₆₆₋₁₇₅, MOG₁₇₂₋₁₈₀, MOG₁₇₉₋₁₈₈, MOG₁₈₈₋₁₉₆, MOG₁₈₁₋₁₈₉, MOG₂₀₅₋₂₁₄, PLP₈₀₋₈₈, MAG₂₈₇₋₂₉₅, MAG₅₀₉₋₅₁₇, MAG₅₅₆₋₅₆₄, MOG₉₇₋₁₀₉ MOG_{97-109(E107S)}, MBP₈₉₋₁₀₁, PLP₁₇₅₋₁₉₂, PLP₉₄₋₁₀₈, MBP₈₆₋₉₈, PLP₅₄₋₆₈, PLP₂₄₉₋₂₆₃, MOG₁₅₆₋₁₇₀, MOG₂₀₁₋₂₁₅, MOG₃₈₋₅₂, MOG₂₀₃₋₂₁₇, PLP₂₅₀₋₂₆₄, MPB₁₃₋₃₂, MPB₈₃₋₉₉, MPB₁₁₁₋₁₂₉, MPB₁₄₆₋₁₇₀, MOG₂₂₃₋₂₃₇, MOG₆₋₂₀, PLP₈₈₋₁₀₂, PLP₁₃₉₋₁₅₄, or a fragment or an equivalent of each thereof;
c) a Celiac Disease-relevant antigen derived from an antigen selected from one or more of the group: aGlia₅₇₋₆₈, aGlia₆₂₋₇₂, aGlia₂₁₇₋₂₂₉, or a fragment or an equivalent of each thereof;
d) a primary biliary cirrhosis-relevant antigen derived from an antigen selected from one or more of the group: PDC-E2₁₂₂₋₁₃₅, PDC-E2₂₄₉₋₂₆₂, PDC-E2₂₄₉₋₂₆₃, PDC-E2₆₂₉₋₆₄₃, PDC-E₂₇₂₋₈₆, PDC-E2₃₅₃₋₃₆₇, PDC-E2₄₂₂₋₄₃₆, PDC-E2₆₂₉₋₆₄₃, PDC-E2₈₀₋₉₄, PDC-E2₃₅₃₋₃₆₇, PDC-E₂₅₃₅₋₅₄₉, or a fragment or an equivalent of each thereof;
e) a pemphigus folliaceus-relevant antigen and/or pemphigus vulgaris-relevant antigen, each of which is derived from an antigen selected from one or more of the group: DG1₂₁₆₋₂₂₉, DG3₉₇₋₁₁₁, DG3₂₅₁₋₂₆₅, DG3₄₄₁₋₄₅₅, DG3₃₅₁₋₃₆₅, DG3₄₅₃₋₄₆₇, DG3₅₄₀₋₅₅₄, DG3₂₈₀₋₂₉₄, DG3₃₂₆₋₃₄₀, DG3₃₆₇₋₃₈₁, DG3₁₃₋₂₇, DG3₃₂₃₋₃₃₇, DG3₄₃₈₋₄₅₂, DG1₄₈₋₆₂, DG1₂₀₆₋₂₂₂, DG1₃₆₃₋₃₇₇, DG1₃₋₁₇, DG1₁₉₂₋₂₀₆, DG1₃₂₆₋₃₄₀, DG1₁₋₁₅, DG1₃₅₋₄₉, DG1₃₂₅₋₃₃₉, or a fragment or an equivalent of each thereof;
f) a neuromyelitis optica spectrum disorder-relevant antigen derived from an antigen selected from one or more of the group: AQP4₁₂₉₋₁₄₃, AQP4₂₈₄₋₂₉₈, AQP4₆₃₋₇₆, AQP4₁₂₉₋₁₄₃, AQP4₃₉₋₅₃, or a fragment or an equivalent of each thereof;
g) an allergic asthma-relevant antigen derived from an antigen selected from one or more of the group: DERP1₁₆₋₃₀, DERP1₁₇₁₋₁₈₅, DERP1₁₁₀₋₁₂₄, DERP-2₂₆₋₄₀, DERP-2₁₀₇₋₁₂₁, or a fragment or an equivalent of each thereof;
h) an inflammatory bowel disease-relevant antigen derived from an antigen selected from one or more of the group: bacteroides integrase antigen₁₈₃₋₁₉₇, bacteroides integrase antigen₁₄₆₋₁₆₀, bacteroides integrase antigen₁₇₅₋₁₈₉, bacteroides integrase antigen₁₋₁₅, bacteroides integrase antigen₁₈₃₋₁₉₇, bacteroides integrase antigen₃₀₋₄₄, bacteroides integrase antigen₇₀₋₈₄, bacteroides integrase antigen₃₃₇₋₃₅₁, bacteroides integrase antigen₁₇₁₋₁₈₅, bacteroides integrase antigen₄₋₁₈, bacteroides integrase antigen₂₅₆₋₂₇₀, Fla-2/Fla-X₃₆₆₋₃₈₀, Fla-2/Fla-X₁₆₄₋₁₇₈, Fla-2/Fla-X₂₆₁₋₂₇₅, Fla-2/Fla-X₁₋₁₅, Fla-2/Fla-X₅₁₋₆₅, Fla-2/Fla-X₂₆₉₋₂₈₃, Fla-2/Fla-X₄₋₁₈, Fla-2/Fla-X₂₇₁₋₂₈₅, YIDX₇₈₋₉₂, YIDX₉₃₋₁₀₇, YIDX₉₈₋₁₁₂, YIDX₂₃₋₃₇, YIDX₇₈₋₉₂, YIDX₁₉₅₋₂₀₉, YIDX₂₂₋₃₆, YIDX₈₀₋₉₄, YIDX₁₀₁₋₁₁₅, or a fragment or an equivalent of each thereof;
i) a systemic lupus erythematosus-relevant antigen derived from an antigen selected from one or more of the group: H4₇₁₋₉₄, H4₇₄₋₈₈, H4₇₆₋₉₀, H4₇₅₋₈₉, H4₇₈₋₉₂, H4₈₀₋₉₄, H2B₁₀₋₂₄, H2B₁₆₋₃₀, H1′₂₂₋₄₂, H1′₂₇₋₄₁, or a fragment or an equivalent of each thereof;
j) an atherosclerosis-relevant antigen derived from an antigen selected from one or more of the group: ApoB₃₅₀₁₋₃₅₁₆, ApoB₁₉₅₂₋₁₉₆₆, ApoB₉₇₈₋₉₉₃, ApoB₃₄₉₈₋₃₅₁₃, ApoB_{210A}, ApoB_{210B}, ApoB_{210C}, or a fragment or an equivalent of each thereof;
k) a COPD-relevant antigen and/or emphysema-relevant antigen, each of which is derived from an antigen selected from one or more of the group: elastin₈₉₋₁₀₃, elastin₆₉₈₋₇₁₂, elastin₈₋₂₂, elastin₉₄₋₁₀₈, elastin₁₃₋₂₇, elastin₆₉₅₋₇₀₉, elastin₅₆₃₋₅₇₇, elastin₅₅₈₋₅₇₂, elastin₆₉₈₋₇₁₂, elastin₅₆₆₋₅₈₀, elastin₆₄₅₋₆₅₉, or a fragment or an equivalent of each thereof;
l) a psoriasis-relevant antigen derived from an antigen selected from one or more of the group: Cap18₆₄₋₇₈, Cap18₃₄₋₄₈, Cap18₄₇₋₆₁, Cap18₁₅₁₋₁₆₅, Cap18₁₄₉₋₁₆₃, Cap18₁₅₂₋₁₆₆, Cap18₁₃₁₋₁₄₅, Cap18₂₄₋₃₈, ADMTSL5245₋₂₅₉, ADMTSL5₂₆₇₋₂₈₁, ADMTSL5₃₇₂₋₃₈₆, ADMTSL5₂₈₉₋₃₀₃, ADMTSL5₃₉₆₋₄₁₀, ADMTSL5₄₃₃₋₄₄₇, ADMTSL5₁₄₂₋₁₅₆, ADMTSL5₂₃₆₋₂₅₀, ADMTSL5₃₀₁₋₃₁₅, ADMTSL5₂₀₃₋₂₁₇, ADMTSL5₄₀₄₋₄₁₈, or a fragment or an equivalent of each thereof;
m) an autoimmune hepatitis-relevant antigen derived from an antigen selected from one or more of the group: (CYP2D6)₁₉₃₋₂₀₇, CYP2D6₇₆₋₉₀, CYP2D6₂₉₃₋₃₀₇, CYP2D6₃₁₃₋₃₃₂, CYP2D6₃₉₃₋₄₁₂, CYP2D6₁₉₉₋₂₁₃, CYP2D6₄₅₀₋₄₆₄, CYP2D6₃₀₁₋₃₁₅, CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₃₁₋₁₄₅, CYP2D6₂₁₆₋₂₃₀, CYP2D6₂₃₈₋₂₅₂, CYP2D6₁₉₉₋₂₁₃, CYP2D6₂₃₅₋₂₅₂, CYP2D6₂₉₃₋₃₀₇, CYP2D6₃₈₁₋₃₉₅, CYP2D6₄₂₉₋₄₄₃, SLA₃₃₄₋₃₄₈, SLA₁₉₆₋₂₁₀, SLA₁₁₅₋₁₂₉, SLA₃₇₃₋₃₈₆, SLA₁₈₆₋₁₉₇, SLA₃₁₇₋₃₃₁, SLA₁₇₁₋₁₈₅, SLA₄₁₇₋₄₃₁, SLA₃₅₉₋₃₇₃, SLA₂₁₅₋₂₂₉, SLA₁₁₁₋₁₂₅, SLA₁₁₀₋₁₂₄, SLA₂₉₉₋₃₁₃, SLA₃₄₂₋₃₅₆, SLA₄₉₋₆₃, SLA₁₁₉₋₁₃₃, SLA₂₆₀₋₂₇₄, SLA₂₆₋₄₀, SLA₈₆₋₁₀₀, SLA₃₃₁₋₃₄₅, or a fragment or an equivalent of each thereof;
n) an uveitis-relevant antigen derived from an antigen selected from one or more of the group: arrestin₁₉₉₋₂₁₃, arrestin₇₇₋₉₁, arrestin₂₅₀₋₂₆₄, arrestin₁₇₂₋₁₈₆, arrestin₃₅₄₋₃₆₈, arrestin₂₃₉₋₂₅₃, arrestin₁₀₂₋₁₁₆, arrestin₅₉₋₇₃, arrestin₂₈₀₋₂₉₄, arrestin₂₉₁₋₃₀₆, arrestin₁₉₅₋₂₀₉, arrestin₂₀₀₋₂₁₄, or a fragment or an equivalent of each thereof;
o) a Sjogren's Syndrome-relevant antigen derived from an antigen selected from one or more of the group: RO60₁₂₇₋₁₄₁, RO60₅₂₃₋₅₃₇, RO60₂₄₃₋₂₅₇, RO60₄₈₄₋₄₉₈, RO60₃₄₇₋₃₆₁, RO60₃₆₉₋₃₈₃, RO60₄₂₆₋₄₄₀, RO60₂₆₇₋₂₈₁, RO60₁₇₈₋₁₉₂, RO60₃₅₈₋₃₇₂, RO60₂₂₁₋₂₃₅, RO60₃₁₈₋₃₃₂, RO60₄₀₇₋₄₂₁, RO60₄₅₉₋₄₇₃, RO60₅₁₋₆₅, RO60₃₁₂₋₃₂₆, LA₂₄₁₋₂₅₅, LA₁₀₁₋₁₁₅, LA₁₅₃₋₁₆₇, LA₁₇₈₋₁₉₂, LA₁₉₋₃₃, LA₃₇₋₅₁, LA₁₃₃₋₁₄₇, LA₅₀₋₆₄, LA₃₂₋₄₆, LA₁₅₃₋₁₆₇, LA₈₃₋₉₇, LA₁₃₆₋₁₅₀, LA₂₉₇₋₃₁₁, LA₅₉₋₇₃, LA₁₅₁₋₁₆₅, LA₈₆₋₁₀₀, LA₁₅₄₋₁₆₈, or a fragment or an equivalent of each thereof;
p) a scleroderma-relevant antigen derived from an antigen selected from one or more of the group: TOP1₃₄₆₋₃₆₀, TOP1₄₂₀₋₄₃₄, TOP1₇₅₀₋₇₆₄, TOP1₄₁₉₋₄₃₃, TOP1₅₉₁₋₆₀₅, TOP1₆₉₅₋₇₀₉, TOP1₃₀₅₋₃₁₉, TOP1₃₄₆₋₃₆₀, TOP1₄₁₉₋₄₃₃, TOP1₄₂₅₋₄₃₉, TOP1₆₁₄₋₆₂₈, CENP-C₂₉₇₋₃₁1, CENP-C₈₅₇₋₈₇₁, CENP-C₈₈₇₋₉₀₁, CENP-C₂₁₂₋₂₂₆, CENP-C₆₄₃₋₆₅₇, CENP-C₈₃₂₋₈₄₆, CENP-C₁₆₇₋₁₈₁, CENP-C₂₄₆₋₂₆₀, CENP-C₈₄₆₋₈₆₀, CENP-C₁₄₉₋₁₆₃, CENP-C₈₃₃₋₈₄₇, CENP-C₈₄₇₋₈₆₁, or a fragment or an equivalent of each thereof;
q) an anti-phospholipid syndrome-relevant antigen derived from an antigen selected from one or more of the group: APOH₂₃₅₋₂₄₉, APOH₃₀₆₋₃₂₀, APOH₂₃₇₋₂₅₁, APOH₂₉₅₋₃₀₉, APOH₂₈₄₂, APOH₁₇₃₋₁₈₇, APOH₂₆₄₋₂₇₈, APOH₂₉₅₋₃₀₉, APOH₄₉₋₆₃, APOH₂₆₉₋₂₈₃, APOH₂₉₅₋₃₀₉, APOH₃₂₁₋₃₅₅, APOH₃₂₂₋₃₃₆, APOH₃₂₄₋₃₃₈, or a fragment or an equivalent of each thereof;
r) an ANCA-associated vasculitis-relevant antigen derived from an antigen selected from one or more of the group: MPO₅₀₆₋₅₂₀, MPO₃₀₂₋₃₁₆, MPO₇₋₂₁, MPO₆₈₉₋₇₀₃, MPO₂₄₈₋₂₆₂, MPO₄₄₄₋₄₅₈, MPO₅₁₃₋₅₂₇, MPO₉₇₋₁₁₁, MPO₆₁₆₋₆₃₀, MPO₄₆₂₋₄₇₆, MPO₆₁₇₋₆₃₁, MPO₇₁₄₋₇₂₈, PRTN3₄₄₋₅₈, PRTN3₂₃₄₋₂₄₈, PRTN3₅₉₋₇₃, PRTN3₁₁₇₋₁₃₁, PRTN3₁₆₄₋₁₇₈, PRTN3₇₁₋₈₅, PRTN3₂₄₁₋₂₅₅, PRTN3₅₉₋₇₃, PRTN3₁₈₃₋₁₉₇, PRTN3₆₂₋₇₆, PRTN3₁₁₈₋₁₃₂, PRTN3₂₃₉₋₂₅₃, or a fragment or an equivalent of each thereof; or
s) a Stiff Man Syndrome-relevant antigen derived from an antigen selected from one or more of the group: GAD₂₁₂₋₂₂₆, GAD₅₅₅₋₅₆₉, GAD₂₉₇₋₃₁₁, or a fragment or an equivalent of each thereof.

In certain aspects, the pMHC complex comprises:
a) a diabetes-relevant antigen derived from an antigen selected from one or more of the group: hInsB₁₀₋₁₈, hIGRP₂₂₈₋₂₃₆, hIGRP₂₆₅₋₂₇₃, IGRP₂₀₆₋₂₁₄, hIGRP₂₀₆₋₂₁₄, NRP-A7, NRP-I4, NRP-V7, YAI/D^{b}, INS B₁₅₋₂₃, PPI_{76-90 (K88S)}, IGRP₁₃₋₂₅, GAD₅₅₅₋₅₆₇, GAD_{555-567(557I)}, IGRP₂₃₋₃₅, B₂₄-C₃₆, PPI₇₆₋₉₀, INS-I9, TUM, G6Pase, Pro-insulin_{L2-10}, Pro-insulin_{L3-11}, Pro-insulin_{L6-14}, Pro-insulin_{B5-14}, Pro-insulin_{B10-18}, Pro-insulin_{B14-22}, Pro-insulin_{B15-24}, Pro-insulin_{B17-25}, Pro-insulin_{B18-27}, Pro-insulin_{B20-27}, Pro-insulin_{B21-29}, Pro-insulin_{B25-C1}, Pro-insulin_{B27-C5}, Pro-insulin_{C20-28}, Pro-insulin_{C25-33}, Pro-insulin_{C29-A5}, Pro-insulin_{A1-10}, Pro-insulin_{A2-10}, Pro-insulin_{A12-20}, or a fragment or an equivalent of each thereof, and the MHC protein of the pMHC complex comprises all or part of HLA-DR or a fragment or an equivalent thereof;
b) a multiple sclerosis-relevant antigen derived from an antigen selected from one or more of the group: MOG₃₅₋₅₅, MOG₃₆₋₅₅, MAG₂₈₇₋₂₉₅, MAG₅₀₉₋₅₁₇, MAG₅₅₆₋₅₆₄, MBP₁₁₀₋₁₁₈, MOG₁₁₄₋₁₂₂, MOG₁₆₆₋₁₇₅, MOG₁₇₂₋₁₈₀, MOG₁₇₉₋₁₈₈, MOG₁₈₈₋₁₉₆, MOG₁₈₈₋₁₈₉, MOG₂₀₅₋₂₁₄, PLP₈₀₋₈₈, MAG₂₈₇₋₂₉₅, MAG₅₀₉₋₅₁₇, MAG₅₅₆₋₅₆₄, MOG₉₇₋₁₀₉ MOG_{97-109(E107S)}, MBP₈₉₁₀₁, PLP₁₇₅₋₁₉₂, PLP₉₄₋₁₀₈, MBP₈₆₋₉₈, PLP₅₄₋₆₈, PLP₂₄₉₋₂₆₃, MOG₁₅₆₋₁₇₀, MOG₂₀₁₋₂₁₅, MOG₃₈₋₅₂, MOG₂₀₃₋₂₁₇, PLP₂₅₀₋₂₆₄, MPB₁₃₋₃₂, MPB₈₃₋₉₉, MPB₁₁₁₋₁₂₉, MPB₁₄₆₋₁₇₀, MOG₂₂₃₋₂₃₇, MOG₆₋₂₀, PLP₈₈₋₁₀₂, PLP₁₃₉₋₁₅₄, or a fragment or an equivalent of each thereof, and the MHC protein of the pMHC complex comprises all or part of HLA-DR or a fragment or an equivalent thereof;
c) a Celiac Disease-relevant antigen derived from an antigen selected from one or more of the group: aGlia₅₇₋₆₈, aGlia₆₂₋₇₂, aGlia₂₁₇₋₂₂₉, or a fragment or an equivalent of each thereof, and the MHC protein of the pMHC complex comprises all or part of HLA-DQ or a fragment or an equivalent thereof;
d) a primary biliary cirrhosis-relevant antigen derived from an antigen selected from one or more of the group: PDC-E2₁₂₂₋₁₃₅, PDC-E2₂₄₉₋₂₆₂, PDC-E2₂₄₉₋₂₆₃, PDC-E2₆₂₉₋₆₄₃, PDC-E₂₇₂₋₈₆, PDC-E2₃₅₃₋₃₆₇, PDC-E2₄₂₂₋₄₃₆, PDC-E2₆₂₉₋₆₄₃, PDC-E2₈₀₋₉₄, PDC-E2₃₅₃₋₃₆₇, PDC-E₂₅₃₅₋₅₄₉, or a fragment or an equivalent of each thereof, and the MHC protein of the pMHC complex comprises all or part of HLA-DR or a fragment of an equivalent thereof;
e) a pemphigus folliaceus-relevant antigen and/or pemphigus vulgaris-relevant antigen, each of which is derived from an antigen selected from one or more of the group: DG1₂₁₆₋₂₂₉, DG3₉₇₋₁₁₁, DG3₂₅₁₋₂₆₅, DG3₄₄₁₋₄₅₅, DG3₃₅₁₋₃₆₅, DG3₄₅₃₋₄₆₇, DG3₅₄₀₋₅₅₄, DG3₂₈₀₋₂₉₄, DG3₃₂₆₋₃₄₀, DG3₃₆₇₋₃₈₁, DG3₁₃₋₂₇, DG3₃₂₃₋₃₃₇, DG3₄₃₈₋₄₅₂, DG1₄₈₋₆₂, DG1₂₀₆₋₂₂₂, DG1₃₆₃₋₃₇₇, DG1₃₋₁₇, DG1₁₉₂₋₂₀₆, DG1₃₂₆₋₃₄₀, DG1₁₋₁₅, DG1₃₅₋₄₉, DG1₃₂₅₋₃₃₉, or a fragment or an equivalent of each thereof, and the MHC protein of the pMHC complex comprises all or part of HLA-DR or a fragment or an equivalent thereof;
f) a neuromyelitis optica spectrum disorder-relevant antigen derived from an antigen selected from one or more of the group: AQP4₁₂₉₋₁₄₃, AQP4₂₈₄₋₂₉₈, AQP4₆₃₋₇₆, AQP4₁₂₉₋₁₄₃, AQP4₃₉₋₅₃, or a fragment or an equivalent of each thereof, and the MHC protein of the pMHC complex comprises all or part of HLA-DR or a fragment or an equivalent thereof;
g) an allergic asthma-relevant antigen derived from an antigen selected from one or more of the group: DERP1₁₆₋₃₀, DERP1₁₇₁₋₁₈₅, DERP1₁₁₀₋₁₂₄, DERP-2₂₆₋₄₀, DERP-2₁₀₇₋₁₂₁, or a fragment or an equivalent of each thereof, and the MHC protein of the pMHC complex comprises all or part of a polypeptide of the group: HLA-DR, HLA-DP, or a fragment or an equivalent of each thereof;
h) an inflammatory bowel disease-relevant antigen derived from an antigen selected from one or more of the group: bacteroides integrase antigen₁₈₃₋₁₉₇, bacteroides integrase antigen₁₄₆₋₁₆₀, bacteroides integrase antigen₁₇₅₋₁₈₉, bacteroides integrase antigen₁₋₁₅, bacteroides integrase antigen₁₈₃₋₁₉₇, bacteroides integrase antigen₃₀₋₄₄, bacteroides integrase antigen₇₀₋₈₄, bacteroides integrase antigen₃₃₇₋₃₅₁, bacteroides integrase antigen₁₇₁₋₁₈₅, bacteroides integrase antigen₄₋₁₈, bacteroides integrase antigen₂₅₆₋₂₇₀, Fla-2/Fla-X₃₆₆₋₃₈₀, Fla-2/Fla-X₁₆₄₋₁₇₈, Fla-2/Fla-X₂₆₁₋₂₇₅, Fla-2/Fla-X₁₋₁₅, Fla-2/Fla-X₅₁₋₆₅, Fla-2/Fla-X₂₆₉₋₂₈₃, Fla-2/Fla-X₄₋₁₈, Fla-2/Fla-X₂₇₁₋₂₈₅, YIDX₇₈₋₉₂, YIDX₉₃₋₁₀₇, YIDX₉₈₋₁₁₂, YIDX₂₃₋₃₇, YIDX₇₈₋₉₂, YIDX₁₉₅₋₂₀₉, YIDX₂₂₋₃₆, YIDX₈₀₋₉₄, YIDX₁₀₁₋₁₁₅, or a fragment or an equivalent of each thereof, and the MHC protein of the pMHC complex comprises all or part of HLA-DR or a fragment or an equivalent thereof;
i) a systemic lupus erythematosus-relevant antigen derived from an antigen selected from one or more of the group: H4₇₁₋₉₄, H4₇₄₋₈₈, H4₇₆₋₉₀, H4₇₅₋₈₉, H4₇₈₋₉₂, H4₈₀₋₉₄, H2B₁₀₋₂₄, H2B₁₆₋₃₀, H1′₂₂₋₄₂, H1′₂₇₋₄₁, or a fragment or an equivalent of each thereof, and the MHC protein of the pMHC complex comprises all or part of a polypeptide of the group: I-A_{d}, HLA-DR, or a fragment or an equivalent of each thereof;
j) an atherosclerosis-relevant antigen derived from an antigen selected from one or more of the group: ApoB₃₅₀₁₋₃₅₁₆, ApoB₁₉₅₂₋₁₉₆₆, ApoB₉₇₈₋₉₉₃, ApoB₃₄₉₈₋₃₅₁₃, ApoB_{210A}, ApoB_{210B}, ApoB_{210C}, or a fragment or an equivalent of each thereof, and the MHC protein of the pMHC complex comprises all or part of I-A_{b} or a fragment or an equivalent thereof;
k) a COPD-relvant antigen and/or emphysema-relevant antigen, each of which is derived from an antigen selected from one or more of the group: elastin₈₉₋₁₀₃, elastin₆₉₈₋₇₁₂, elastin₈₋₂₂, elastin₉₄₋₁₀₈, elastin₁₃₋₂₇, elastin₆₉₅₋₇₀₉, elastin₅₆₃₋₅₇₇, elastin₅₅₈₋₅₇₂, elastin₆₉₈₋₇₁₂, elastin₅₆₆₋₅₈₀, elastin₅₄₅₋₆₅₉, or a fragment or an equivalent of each thereof, and the MHC protein of the pMHC complex comprises all or part of HLA-DR or a fragment or an equivalent thereof;
l) a psoriasis-relevant antigen derived from an antigen selected from one or more of the group: Cap18₆₄₋₇₈, Cap18₃₄₋₄₈, Cap18₄₇₋₆₁, Cap18₁₅₁₋₁₆₅, Cap18₁₄₉₋₁₆₃, Cap18₁₅₂₋₁₆₆, Cap18₁₃₁₋₁₄₅, Cap18₂₄₋₃₈, ADMTSL5₂₄₅₋₂₅₉, ADMTSL5₂₆₇₋₂₈₁, ADMTSL5₃₇₂₋₃₈₆, ADMTSL5₂₈₉₋₃₀₃, ADMTSL5₃₉₆₋₄₁₀, ADMTSL5₄₃₃₋₄₄₇, ADMTSL5₁₄₂₋₁₅₆, ADMTSL5₂₃₆₋₂₅₀, ADMTSL5₃₀₁₋₃₁₅, ADMTSL5₂₀₃₋₂₁₇, ADMTSL5₄₀₄₋₄₁₈, or a fragment or an equivalent of each thereof, and the MHC protein of the pMHC complex comprises all or part of HLA-DR or a fragment or an equivalent thereof;
m) an autoimmune hepatitis-relevant antigen derived from an antigen selected from one or more of the group: CYP2D6₁₉₃₋₂₀₇, CYP2D6₇₆₋₉₀, CYP2D6₂₉₃₋₃₀₇, CYP2D6₃₁₃₋₃₃₂, CYP2D6₃₉₃₋₄₁₂, CYP2D6₁₉₉₋₂₁₃, CYP2D6₄₅₀₋₄₆₄, CYP2D6₃₀₁₋₃₁₅, CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₃₁₋₁₄₅, CYP2D6₂₁₆₋₂₃₀, CYP2D6₂₃₈₋₂₅₂, CYP2D6₁₉₉₋₂₁₃, CYP2D6₂₃₅₋₂₅₂, CYP2D6₂₉₃₋₃₀₇, CYP2D6₃₈₁₋₃₉₅, CYP2D6₄₂₉₋₄₄₃, SLA₃₃₄₋₃₄₈, SLA₁₉₆₋₂₁₀, SLA₁₁₅₋₁₂₉, SLA₃₇₃₋₃₈₆, SLA₁₈₆₋₁₉₇, SLA₃₁₇₋₃₃₁, SLA₁₇₁₋₁₈₅, SLA₄₁₇₋₄₃₁, SLA₃₅₉₋₃₇₃, SLA₂₁₅₋₂₂₉, SLA₁₁₁₋₁₂₅, SLA₁₁₀₋₁₂₄, SLA₂₉₉₋₃₁₃, SLA₃₄₂₋₃₅₆, SLA₄₉₋₆₃, SLA₁₁₉₋₁₃₃, SLA₂₆₀₋₂₇₄, SLA₂₆₋₄₀, SLA₈₆₋₁₀₀, SLA₃₃₁₋₃₄₅, or a fragment or an equivalent of each thereof, and the MHC protein of the pMHC complex comprises all or part of HLA-DR or a fragment or an equivalent thereof;
n) an uveitis-relevant antigen derived from an antigen selected from one or more of the group: arrestin₁₉₉₋₂₁₃, arrestin₇₇₋₉₁, arrestin₂₅₀₋₂₆₄, arrestin₁₇₂₋₁₈₆, arrestin₃₅₄₋₃₆₈, arrestin₂₃₉₋₂₅₃, arrestin₁₀₂₋₁₁₆, arrestin₅₉₋₇₃, arrestin₂₈₀₋₂₉₄, arrestin₂₉₁₋₃₀₆, arrestin₁₉₅₋₂₀₉, arrestin₂₀₀₋₂₁₄, or a fragment or an equivalent of each thereof, and the MHC protein of the pMHC complex comprises all or part of HLA-DR or a fragment or an equivalent thereof;
o) a Sjogren's Syndrome-relevant antigen derived from an antigen selected from one or more of the group: RO60₁₂₇₋₁₄₁, RO60₅₂₃₋₅₃₇, RO60₂₄₃₋₂₅₇, RO60₄₈₄₋₄₉₈, RO60₃₄₇₋₃₆₁, RO60₃₆₉₋₃₈₃, RO60₄₂₆₋₄₄₀, RO60₂₆₇₋₂₈₁, RO60₁₇₈₋₁₉₂, RO60₃₅₈₋₃₇₂, RO60₂₂₁₋₂₃₅, RO60₃₁₈₋₃₃₂, RO60₄₀₇₋₄₂₁, RO60₄₅₉₋₄₇₃, RO60₅₁₋₆₅, RO60₃₁₂₋₃₂₆, LA₂₄₁₋₂₅₅, LA₁₀₁₋₁₁₅, LA₁₅₃₋₁₆₇, LA₁₇₈₋₁₉₂, LA₁₉₋₃₃, LA₃₇₋₅₁, LA₁₃₃₋₁₄₇, LA₅₀₋₆₄, LA₃₂₋₄₆, LA₁₅₃₋₁₆₇, LA₈₃₋₉₇, LA₁₃₆₋₁₅₀, LA₂₉₇₋₃₁₁, LA₅₉₋₇₃, LA₁₅₁₋₁₆₅, LA₈₆₋₁₀₀, LA₁₅₄₋₁₆₈, or a fragment or an equivalent of each thereof, and the MHC protein of the pMHC complex comprises all or part of a polypeptide of the group: HLA-DR, HLA-DP, or a fragment or an equivalent of each thereof;
p) a scleroderma-relevant antigen derived from an antigen selected from one or more of the group: TOP1₃₄₆₋₃₆₀, TOP1₄₂₀₋₄₃₄, TOP1₇₅₀₋₇₆₄, TOP1₄₁₉₋₄₃₃, TOP1₅₉₁₋₆₀₅, TOP1₆₉₅₋₇₀₉, TOP1₃₀₅₋₃₁₉, TOP1₃₄₆₋₃₆₀, TOP1₄₁₉₋₄₃₃, TOP1₄₂₅₋₄₃₉, TOP1₆₁₄₋₆₂₈, CENP-C₂₉₇₋₃₁₁, CENP-C₈₅₇₋₈₇₁, CENP-C₈₈₇₋₉₀₁, CENP-C₂₁₂₋₂₂₆, CENP-C₆₄₃₋₆₅₇, CENP-C₈₃₂₋₈₄₆, CENP-C₁₆₇₋₁₈₁, CENP-C₂₄₆₋₂₆₀, CENP-C₈₄₆₋₈₆₀, CENP-C₁₄₉₋₁₆₃, CENP-C₈₃₃₋₈₄₇, CENP-C₈₄₇₋₈₆₁, or a fragment or an equivalent of each thereof, and the MHC protein of the pMHC complex comprises all or part of HLA-DR or a fragment or an equivalent thereof;
q) an anti-phospholipid syndrome-relevant antigen derived from an antigen selected from one or more of the group: APOH₂₃₅₋₂₄₉, APOH₃₀₆₋₃₂₀, APOH₂₃₇₋₂₅₁, APOH₂₉₅₋₃₀₉, APOH₂₈₄₂, APOH₁₇₃₋₁₈₇, APOH₂₆₄₋₂₇₈, APOH₂₉₅₋₃₀₉, APOH₄₉₋₆₃, APOH₂₆₉₋₂₈₃, APOH₂₉₅₋₃₀₉, APOH₃₂₁₋₃₅₅, APOH₃₂₂₋₃₃₆, APOH₃₂₄₋₃₃₈, or a fragment or an equivalent of each thereof, and the MHC protein of the pMHC complex comprises all or part of HLA-DR or a fragment or an equivalent thereof;
r) an ANCA-associated vasculitis-relevant antigen derived from an antigen selected from one or more of the group: MPO₅₀₆₋₅₂₀, MPO₃₀₂₋₃₁₆, MPO₇₋₂₁, MPO₆₈₉₋₇₀₃, MPO₂₄₈₋₂₆₂, MPO₄₄₄₋₄₅₈, MPO₅₁₃₋₅₂₇, MPO₉₇₋₁₁₁, MPO₆₁₆₋₆₃₀, MPO₄₆₂₋₄₇₆, MPO₆₁₇₋₆₃₁, MPO₇₁₄₋₇₂₈, PRTN3₄₄₋₅₈, PRTN3₂₃₄₋₂₄₈, PRTN3₅₉₋₇₃, PRTN3₁₁₇₋₁₃₁, PRTN3₁₆₄₋₁₇₈, PRTN3₇₁₋₈₅, PRTN3₂₄₁₋₂₅₅, PRTN3₅₉₋₇₃, PRTN3₁₈₃₋₁₉₇, PRTN3₆₂₋₇₆, PRTN3₁₁₈₋₁₃₂, PRTN3₂₃₉₋₂₅₃, or a fragment or an equivalent of each thereof, and the MHC protein of the pMHC complex comprises all or part of HLA-DR or a fragment or an equivalent thereof; or
s) a Stiff Man Syndrome-relevant antigen derived from an antigen selected from one or more of the group: GAD₂₁₂₋₂₂₆, GAD₅₅₅₋₅₆₉, GAD₂₉₇₋₃₁₁, and the MHC protein of the pMHC complex comprises all or part of a polypeptide of the group: HLA-DR, HLA-DQ, or a fragment or an equivalent of each thereof.

In certain aspects, the pMHC complex is for the treatment of:
a) type I diabetes and the pMHC complex is selected from the group of: PPI_{76-90(K88S)}-HLA-DRB1*0401/DRA, IGRP₁₃₋₂₅-HLA-DRB1*0301/DRA, GAD₅₅₅₋₅₆₇-HLA-DRB1*0401/DRA, GAD_{555-567(557I)}-HLA-DRB1*0401/DRA, IGRP₂₃₋₃₅-HLA-DRB1*0401/DRA, B₂₄-C₃₆-HLA-DRB1*0301/DRA, or PPI₇₆₋₉₀-HLA-DRB1*0401/DRA;
b) multiple sclerosis and the pMHC complex is selected from the group of: MBP₈₆₋₉₈-HLA-DRB1*1501/DRA, MBP₈₉₋₁₀₁-HLA-DRB5*0101/DRA, MOG₃₈₋₅₂-HLA-DRB4*0101/DRA, MOG_{97-109(E107S)}-HLA-DRB1*0401/DRA, MOG₂₀₃₋₂₁₇-HLA-DRB3*0101/DRA, PLP₅₄₋₆₅-HLA-DRB3*0101/DRA, PLP₉₄₋₁₀₈-HLA-DRB1*0301/DRA, PLP₂₅₀₋₂₆₄-HLA-DRB4*0101/DRA, MPB₁₃₋₃₂-HLA-DRB5*0101/DRA, MPB₈₃₋₉₉-HLA-DRB5*0101/DRA, MPB₁₁₁₋₁₂₉-HLA-DRB5*0101/DRA, MPB₁₄₆₋₁₇₀-HLA-DRB5*0101/DRA, MOG₂₂₃₋₂₃₇-HLA-DRB3*0202/DRA, MOG₆₋₂₀-HLA-DRB5*0101/DRA, PLP₈₈₋₁₀₂-HLA-DRB3*0202/DRA, or PLP₁₃₉₋₁₅₄-HLA-DRB5*0101/DRA;
c) Celiac Disease and the pMHC complex is selected from the group of: aGlia₅₇₋₆₈-HLA-DQA1*0501/HLA-DQB1*0201, aGlia₆₂₋₇₂-HLA-DQA1*0501/HLA-DQB1*0201, aGlia₂₁₇₋₂₂₉-HLA-DQA1*0501/HLA-DQB1*0302, or aGlia₂₁₇₋₂₂₉-HLA-DQA1*03/HLA-DQB1*0302;
d) primary biliary cirrhosis and the pMHC complex is selected from the group of: PDC-E2₁₂₂₋₁₃₅-HLA-DRB4*0101/DRA, PDC-E2₂₄₉₋₂₆₂-HLA-DRB4*0101/DRA, PDC-E2₂₄₉₋₂₆₃-HLA-DRB1*0801/DRA, PDC-E2₆₂₉₋₆₄₃-HLA-DRB1*0801/DRA, PDC-E2₇₂₋₈₆-HLA-DRB3*0202/DRA, PDC-E2₅₃₋₃₆₇-HLA-DRB3*0202/DRA, PDC-E2₄₂₂₋₄₃₆-HLA-DRB3*0202/DRA, PDC-E2₆₂₉₋₆₄₃-HLA-DRB4*0101/DRA, PDC-E2₈₀₋₉₄-HLA-DRB5*0101/DRA, PDC-E2₅₃₋₃₆₇-HLA-DRB5*0101/DRA, or PDC-E2₅₃₅₋₅₄₉-HLA-DRB5*0101/DRA, mPDC-E2₁₆₆₋₁₈₁-I-A_{g7}, or mPDC-E2₈₂₋₉₆-I-A_{g7};
e) pemphigus folliaceus and/or pemphigus vulgaris and the pMHC complex is selected from the group of: DG1₂₁₆₋₂₂₉-HLA-DRB1*0101/DRA, DG1₂₁₆₋₂₂₉-HLA-DRB1*0102/DRA, DG3₉₇₋₁₁₁-HLA-DRB1*0402/DRA, DG3₂₅₁₋₂₆₅-HLA-DRB1*0402/DRA, DG3₂₅₁₋₂₆₅-HLA-DRB1*0401/DRA, DG3₄₄₁₋₄₅₅-HLA-DRB1*0402/DRA, DG3₃₅₁₋₃₆₅-HLA-DRB3*0202/DRA, DG3₄₅₃₋₄₆₇-HLA-DRB3*0202/DRA, DG3₅₄₀₋₅₅₄-HLA-DRB3*0202/DRA, DG3₂₈₀₋₂₉₄-HLA-DRB4*0101/DRA, DG3₃₂₆₋₃₄₀-HLA-DRB4*0101/DRA, DG3₃₆₇₋₃₈₁-HLA-DRB4*0101/DRA, DG3₁₃₋₂₇-HLA-DRB5*0101/DRA, DG3₃₂₃₋₃₃₇-HLA-DRB5*0101/DRA, DG3₄₃₈₋₄₅₂-HLA-DRB5*0101/DRA, DG1₄₈₋₆₂-HLA-DRB3*0202/DRA, DG1₂₀₆₋₂₂₂-HLA-DRB3*0202/DRA, DG1₃₆₃₋₃₇₇-HLA-DRB3*0202/DRA, DG1₃₋₁₇-HLA-DRB4*0101/DRA, DG1₁₉₂₋₂₀₆-HLA-DRB4*0101/DRA, DG1₃₂₆₋₃₄₀-HLA-DRB4*0101/DRA, DG1₁₋₁₅-HLA-DRB5*0101/DRA, DG1₃₅₋₄₉-HLA-DRB5*0101/DRA, or DG1₃₂₅₋₃₃₉-HLA-DRB5*0101/DRA;
f) neuromyelitis optica spectrum disorder and the pMHC complex is selected from the group of: AQP4₁₂₉₋₁₄₃-HLA-DRB1*0101/DRA, AQP4₂₈₄₋₂₉₈-HLA-DRB1*0301/DRA, AQP4₆₃₋₇₆-HLA-DRB1*0301/DRA, AQP4₁₂₉₋₁₄₃-HLA-DRB1*0401/DRA, or AQP4₃₉₋₅₃-HLA-DRB1*1501/DRA;
g) allergic asthma and the pMHC complex is selected from the group of: DERP-1₁₆₋₃₀-HLA-DRB1*0101/DRA, DERP-1₁₆₋₃₀-HLA-DRB1*1501/DRA, DERP₁₁₇₁₋₁₈₅-HLA-DRB1*1501/DRA, DERP-1₁₁₀₋₁₂₄-HLA-DPB1*0401/DRA, DERP-2₂₆₋₄₀-HLA-DRB1*0101/DRA; DERP-2₂₆₋₄₀-HLA-DRB1*1501/DRA, or DERP-2₁₀₇₋₁₂₁-HLA-DRB1*0301/DRA;
h) inflammatory bowel disease and the pMHC complex is selected from the group of: bacteroides integrase antigen₁₈₃₋₁₉₇-HLA-DRB3*0101/DRA, bacteroides integrase antigen₁₄₆₋₁₆₀-HLA-DRB3*0101/DRA, bacteroides integrase antigen₁₇₅₋₁₈₉-HLA-DRB3*0101/DRA, bacteroides integrase antigen₁₋₁₅-HLA-DRB5*0101/DRA, bacteroides integrase antigen₁₈₃₋₁₉₇-HLA-DRB5*0101/DRA, bacteroides integrase antigen₁₈₃₋₁₉₇-HLA-DRB3*0101/DRA, bacteroides integrase antigen₃₀₋₄₄-HLA-DRB5*0101/DRA, bacteroides integrase antigen₇₀₋₈₄-HLA-DRB4*0101/DRA, bacteroides integrase antigen₃₃₇₋₃₅₁-HLA-DRB4*0101/DRA, bacteroides integrase antigen₁₇₁₋₁₈₅-HLA-DRB4*0101/DRA, bacteroides integrase antigen₄-is-HLA-DRB3*0202/DRA, bacteroides integrase antigen₁₇₁₋₁₈₅-HLA-DRB3*0202/DRA, bacteroides integrase antigen₂₅₆₋₂₇₀-HLA-DRB3*0202/DRA, Fla-2/Fla-X₃₆₆₋₃₈₀-HLA-DRB3*0101/DRA, Fla-2/Fla-X₁₆₄₋₁₇₈-HLA-DRB3*0101/DRA, Fla-2/Fla-X₂₆₁₋₂₇₅-HLA-DRB5*0101/DRA, Fla-2/Fla-X₁₋₁₅-HLA-DRB5*0101/DRA, Fla-2/Fla-X₅₁₋₆₅-HLA-DRB4*0101/DRA, Fla-2/Fla-X₂₆₉₋₂₈₃-HLA-DRB4*0101/DRA, Fla-2/Fla-X₄₋₁₈-HLA-DRB3*0202/DRA, Fla-2/Fla-X₂₆₁₋₂₇₅-HLA-DRB3*0202/DRA, Fla-2/Fla-X₂₇₁₋₂₈₅-HLA-DRB3*0202/DRA, YIDX₇₈₋₉₂-HLA-DRB3*0101/DRA, YIDX₇₈₋₉₂-HLA-DRB4*0101/DRA, YIDX₉₃₋₁₀₇-HLA-DRB3*0101/DRA, YIDX₉₈₋₁₁₂-HLA-DRB5*0101/DRA, YIDX₂₃₋₃₇-HLA-DRB5*0101/DRA, YIDX₇₈₋₉₂-HLA-DRB4*0101/DRA, YIDX₁₉₅₋₂₀₉-HLA-DRB4*0101/DRA, YIDX₂₂₋₃₆-HLA-DRB3*0202/DRA, YIDX₈₀₋₉₄-HLA-DRB3*0202/DRA, or YIDX₁₀₁₋₁₁₅-HLA-DRB3*0202/DRA;
i) COPD and/or emphysema and the pMHC complex is selected from the group of: elastin₈₉₋₁₀₃-HLA-DRB3*0101/DRA, elastin₆₉₈₋₇₁₂-HLA-DRB5*0101/DRA, elastin₈₋₂₂-HLA-DRB5*0101/DRA, elastin₉₄₋₁₀₈-HLA-DRB5*0101/DRA, elastin₁₃₋₂₇-HLA-DRB4*0101/DRA, elastin₆₉₅₋₇₀₉-HLA-DRB4*0101/DRA, elastin₅₆₃₋₅₇₇-HLA-DRB4*0101/DRA, elastin₅₅₈₋₅₇₂-HLA-DRB4*0101/DRA, elastin₆₉₈₋₇₁₂-HLA-DRB5*0101/DRA, elastin₅₆₆₋₅₈₀-HLA-DRB3*0202/DRA, or elastin₅₄₅₋₆₅₉-HLA-DRB3*0202/DRA;
j) psoriasis and the pMHC complex is selected from the group of: Cap18₆₄₋₇₈-HLA-DRB3*0101/DRA, Cap18₃₄₋₄₈-HLA-DRB3*0101/DRA, Cap18₄₇₋₆₁-HLA-DRB3*0101/DRA, Cap18₁₅₁₋₁₆₅-HLA-DRB4*0101/DRA, Cap18₁₄₉₋₁₆₃-HLA-DRB5*0101/DRA, Cap18₁₅₂₋₁₆₆-HLA-DRB5*0101/DRA, Cap18₁₃₁₋₁₄₅-HLA-DRB5*0101/DRA, Cap18₂₄₋₃₈-HLA-DRB3*0202/DRA, ADMTSL5₂₄₅₋₂₅₉-HLA-DRB3*0101/DRA, ADMTSL5₂₆₇₋₂₈₁-HLA-DRB3*0101/DRA, ADMTSL5₃₇₂₋₃₈₆-HLA-DRB3*0101/DRA, ADMTSL5₂₈₉₋₃₀₃-HLA-DRB4*0101/DRA, ADMTSL5₃₉₆₋₄₁₀-HLA-DRB4*0101/DRA, ADMTSL5₄₃₃₋₄₄₇-HLA-DRB4*0101/DRA, ADMTSL5₁₄₂₋₁₅₆-HLA-DRB5*0101/DRA, ADMTSL5₂₃₆₋₂₅₀-HLA-DRB5*0101/DRA, ADMTSL5₃₀₁₋₃₁₅-HLA-DRB5*0101/DRA, ADMTSL5₂₀₃₋₂₁₇-HLA-DRB3*0202/DRA, ADMTSL5₄₀₄₋₄₁₈-HLA-DRB3*0202/DRA, or ADMTSL5₄₃₃₋₄₄₇-HLA-DRB3*0202/DRA;
k) autoimmune hepatitis and the pMHC complex is selected from the group of: CYP2D6₁₉₃₋₂₀₇-HLA-DRB1*0301/DRA, CYP2D6₇₆₋₉₀-HLA-DRB1*0301/DRA, CYP2D6₂₉₃₋₃₀₇-HLA-DRB1*0301/DRA, CYP2D6₃₁₃₋₃₃₂-HLA-DRB1*0301/DRA, CYP2D6₃₉₃₋₄₁₂-HLA-DRB1*0301/DRA, CYP2D6₁₉₉₋₂₁₃-HLA-DRB1*0401/DRA, CYP2D6₄₅₀₋₄₆₄-HLA-DRB1*0401/DRA, CYP2D6₃₀₁₋₃₁₅-HLA-DRB1*0401/DRA, CYP2D6₄₅₂₋₄₆₆-HLA-DRB1*0701/DRA, CYP2D6₅₉₋₇₃-HLA-DRB1*0701/DRA, CYP2D6₁₃₀₋₁₄₄-HLA-DRB1*0701/DRA, CYP2D6₁₉₃₋₂₁₂-HLA-DRB1*0701/DRA, CYP2D6₃₀₅₋₃₂₄-HLA-DRB1*0701/DRA, CYP2D6₁₃₁₋₁₄₅-HLA-DRB3*0202/DRA, CYP2D6₂₁₆₋₂₃₀-HLA-DRB3*0202/DRA, CYP2D6₂₃₈₋₂₅₂-HLA-DRB3*0202/DRA, CYP2D6₁₉₉₋₂₁₃-HLA-DRB4*0101/DRA, CYP2D6₂₃₅₋₂₅₂-HLA-DRB4*0101/DRA, CYP2D6₂₉₃₋₃₀₇-HLA-DRB4*0101/DRA, CYP2D6₂₃₈₋₂₅₂-HLA-DRB5*0101/DRA, CYP2D6₃₈₁₋₃₉₅-HLA-DRB5*0101/DRA, CYP2D6₄₂₉₋₄₄₃-HLA-DRB5*0101/DRA, SLA₃₃₄₋₃₄₈-HLA-DRB1*0301/DRA, SLA₁₉₆₋₂₁₀-HLA-DRB1*0301/DRA, SLA₁₁₅₋₁₂₉-HLA-DRB1*0301/DRA, SLA₃₇₃₋₃₈₆-HLA-DRB1*0301/DRA, SLA₁₈₆₋₁₉₇-HLA-DRB1*0301/DRA, SLA₃₁₇₋₃₃₁-HLA-DRB1*0401/DRA, SLA₁₇₁₋₁₈₅-HLA-DRB1*0401/DRA, SLA₄₁₇₋₄₃₁-HLA-DRB1*0401/DRA, SLA₃₅₉₋₃₇₃-HLA-DRB1*0701/DRA, SLA₂₁₅₋₂₂₉-HLA-DRB1*0701/DRA, SLA₁₁₁₋₁₂₅-HLA-DRB1*0701/DRA, SLA₁₁₀₋₁₂₄-HLA-DRB3*0202/DRA, SLA₂₉₉₋₃₁₃-HLA-DRB3*0202/DRA, SLA₃₄₂₋₃₅₆-HLA-DRB3*0202/DRA, SLA₄₉₋₆₃-HLA-DRB4*0101/DRA, SLA₁₁₉₋₁₃₃-HLA-DRB4*0101/DRA, SLA₂₆₀₋₂₇₄-HLA-DRB4*0101/DRA, SLA₂₆₋₄₀-HLA-DRB5*0101/DRA, SLA₈₆₋₁₀₀-HLA-DRB5*0101/DRA, or SLA₃₃₁₋₃₄₅-HLA-DRB5*0101/DRA;
l) uveitis and the pMHC complex is selected from the group of: arrestin₁₉₉₋₂₁₃-HLA-DRB3*0101/DRA, arrestin₇₇₋₉₁-HLA-DRB3*0101/DRA, arrestin₂₅₀₋₂₆₄-HLA-DRB3*0101/DRA, arrestin₁₇₂₋₁₈₆-HLA-DRB4*0101/DRA, arrestin₃₅₄₋₃₆₈-HLA-DRB4*0101/DRA, arrestin₂₃₉₋₂₅₃-HLA-DRB4*0101/DRA, arrestin₁₀₂₋₁₁₆-HLA-DRB5*0101/DRA, arrestin₅₉₋₇₃-HLA-DRB5*0101, arrestin₂₈₀₋₂₉₄-HLA-DRB5*0101, arrestin₂₉₁₋₃₀₆-HLA-DRB1*0301/DRA, arrestin₁₉₅₋₂₀₉-HLA-DRB3*0202/DRA, arrestin₁₉₉₋₂₁₃-HLA-DRB3*0202/DRA, or arrestin₂₀₀₋₂₁₄-HLA-DRB3*0202/DRA;
m) Sjogren Syndrome and the pMHC complex is selected from the group of: RO60₁₂₇₋₁₄₁-HLA-DRB1*0301/DRA, RO60₅₂₃₋₅₃₇-HLA-DRB1*0301/DRA, RO60₂₄₃₋₂₅₇-HLA-DRB1*0301/DRA, RO60₄₈₄₋₄₉₈-HLA-DRB3*0101/DRA, RO60₃₄₇₋₃₆₁-HLA-DRB3*0101/DRA, RO60₃₆₉₋₃₈₃-HLA-DRB3*0101/DRA, RO60₄₂₆₋₄₄₀-HLA-DRB4*0101/DRA, RO60₂₆₇₋₂₈₁-HLA-DRB4*0101/DRA, RO60₁₇₈₋₁₉₂-HLA-DRB4*0101/DRA, RO60₃₅₈₋₃₇₂-HLA-DRB5*0101/DRA, RO60₃₅₈₋₃₇₂-HLA-DRB4*0101/DRA, RO60₂₂₁₋₂₃₅-HLA-DRB5*0101/DRA, RO60₂₂₁₋₂₃₅-HLA-DRB4*0101/DRA, RO60₃₁₈₋₃₃₂-HLA-DRB5*0101/DRA, RO60₃₁₈₋₃₃₂-HLA-DRB4*0101/DRA, RO60₄₀₇₋₄₂₁-HLA-DRB4*0101/DRA, RO60₄₀₇₋₄₂₁-HLA-DQA1*0501/HLA-DQB1*0201, RO60₄₅₉₋₄₇₃-HLA-DRB4*0101/DRA, RO60₄₅₉₋₄₇₃-HLA-DQA1*0501/HLA-DQB1*0201, RO60₃₁₈₋₃₃₂-HLA-DQA1*0501/HLA-DQB1*0201, RO60₅₁₋₆₅-HLA-DRB3*0202/DRA, RO60₃₁₂₋₃₂₆-HLA-DRB3*0202/DRA, RO60₃₄₇₋₃₆₁-HLA-DRB3*0202/DRA, LA₂₄₁₋₂₅₅-HLA-DRB1*0301/DRA, LA₁₀₁₋₁₁₅-HLA-DRB1*0301/DRA, LA₁₅₃₋₁₆₇-HLA-DRB1*0301/DRA, LA₁₇₈₋₁₉₂-HLA-DRB3*0101/DRA, LA₁₉₋₃₃-HLA-DRB3*0101/DRA, LA₃₇₋₅₁-HLA-DRB3*0101/DRA, LA₁₃₃₋₁₄₇-HLA-DRB4*0101/DRA, LA₅₀₋₆₄-HLA-DRB4*0101/DRA, LA₃₂₄₆-HLA-DRB4*0101/DRA, LA₁₅₃₋₁₆₇-HLA-DRB5*0101/DRA, LA₈₃₋₉₇-HLA-DRB5*0101/DRA, LA₁₃₆₋₁₅₀-HLA-DRB5*0101/DRA, LA₂₉₇₋₃₁₁-HLA-DQA1*0501/HLA-DQB1*0201, LA₅₉₋₇₃-HLA-DQA1*0501/HLA-DQB1*0201, LA₅₉₋₇₃-HLA-DRB4*0101/DRA, LA₁₅₁₋₁₆₅-HLA-DQA1*0501/HLA-DQB1*0201, LA₁₅₁₋₁₆₅-HLA-DRB4*0101/DRA, LA₂₉₇₋₃₁₁-HLA-DRB4*0101/DRA, LA₅₀₋₆₄-HLA-DRB3*0202/DRA, LA₈₆₋₁₀₀-HLA-DRB3*0202/DRA, or LA₁₅₄₋₁₆₈-HLA-DRB3*0202/DRA;
n) scleroderma and the pMHC complex is selected from the group of: TOP1₃₄₆₋₃₆₀-HLA-DRB3*0101/DRA, TOP1₄₂₀₋₄₃₄-HLA-DRB3*0101/DRA, TOP1₇₅₀₋₇₆₄-HLA-DRB3*0101/DRA, TOP1₄₁₉₋₄₃₃-HLA-DRB4*0101/DRA, TOP1₅₉₁₋₆₀₅-HLA-DRB4*0101/DRA, TOP1₆₉₅₋₇₀₉-HLA-DRB4*0101/DRA, TOP1₃₀₅₋₃₁₉-HLA-DRB5*0101/DRA, TOP1₃₄₆₋₃₆₀-HLA-DRB5*0101/DRA, TOP1₄₁₉₋₄₃₃-HLA-DRB5*0101/DRA, TOP1₄₂₀₋₄₃₄-HLA-DRB3*0202/DRA, TOP1₄₂₅₋₄₃₉-HLA-DRB3*0202/DRA, TOP1₆₁₄₋₆₂₈-HLA-DRB3*0202/DRA, CENP-C₂₉₇₋₃₁₁-HLA-DRB3*0101/DRA, CENP-C₈₅₇₋₈₇₁-HLA-DRB3*0101/DRA, CENP-C₈₈₇₋₉₀₁-HLA-DRB3*0101/DRA, CENP-C₂₁₂₋₂₂₆-HLA-DRB4*0101/DRA, CENP-C₆₄₃₋₆₅₇-HLA-DRB4*0101/DRA, CENP-C₈₃₂₋₈₄₆-HLA-DRB4*0101/DRA, CENP-C₁₆₇₋₁₈₁-HLA-DRB5*0101/DRA, CENP-C₂₄₆₋₂₆₀-HLA-DRB5*0101/DRA, CENP-C₈₄₆₋₈₆₀-HLA-DRB5*0101/DRA, CENP-C₁₄₉₋₁₆₃-HLA-DRB3*0202/DRA, CENP-C₈₃₃₋₈₄₇-HLA-DRB3*0202/DRA, or CENP-C₈₄₇₋₈₆₁-HLA-DRB3*0202/DRA;
o) anti-phospholipid syndrome and the pMHC complex is selected from the group of: APOH₂₃₅₋₂₄₉-HLA-DRB3*0101/DRA, APOH₃₀₆₋₃₂₀-HLA-DRB3*0101/DRA, APOH₂₃₇₋₂₅₁-HLA-DRB3*0101/DRA, APOH₂₉₅₋₃₀₉-HLA-DRB3*0101/DRA, APOH₂₈₋₄₂-HLA-DRB4*0101/DRA, APOH₁₇₃₋₁₈₇-HLA-DRB4*0101/DRA, APOH₂₆₄₋₂₇₈-HLA-DRB4*0101/DRA, APOH₂₉₅₋₃₀₉-HLA-DRB4*0101/DRA, APOH₄₉₋₆₃-HLA-DRB5*0101/DRA, APOH₂₆₉₋₂₈₃-HLA-DRB5*0101/DRA, APOH₂₉₅₋₃₀₉-HLA-DRB5*0101/DRA, APOH₃₂₁₋₃₅₅-HLA-DRB3*0202/DRA, APOH₃₂₂₋₃₃₆-HLA-DRB3*0202/DRA, or APOH₃₂₄₋₃₃₈-HLA-DRB3*0202/DRA;
p) ANCA-associated vasculitis and the pMHC complex is selected from the group of: MPO₅₀₆₋₅₂₀-HLA-DRB3*0101/DRA, MPO₃₀₂₋₃₁₆-HLA-DRB3*0101/DRA, MPO₇₋₂₁-HLA-DRB3*0101/DRA, MPO₆₈₉₋₇₀₃-HLA-DRB4*0101/DRA, MPO₂₄₈₋₂₆₂-HLA-DRB4*0101/DRA, MPO₄₄₄₋₄₅₈-HLA-DRB4*0101/DRA, MPO₅₁₃₋₅₂₇-HLA-DRB5*0101/DRA, MPO₉₇₋₁₁₁-HLA-DRB5*0101/DRA, MPO₆₁₆₋₆₃₀-HLA-DRB5*0101/DRA, MPO₄₆₂₋₄₇₆-HLA-DRB3*0202/DRA, MPO₆₁₇₋₆₃₁-HLA-DRB3*0202/DRA, MPO₇₁₄₋₇₂₈-HLA-DRB3*0202/DRA, PRTN3₄₄₋₅₈-HLA-DRB3*0101/DRA, PRTN3₂₃₄₋₂₄₈-HLA-DRB3*0101/DRA, PRTN3₅₉₋₇₃-HLA DRB3*0101/DRA, PRTN3₅₉₋₇₃-HLA-DRB5*0101/DRA, PRTN3₁₁₇₋₁₃₁-HLA-DRB4*0101/DRA, PRTN3₁₆₄₋₁₇₈-HLA-DRB4*0101/DRA, PRTN3₇₁₋₈₅-HLA-DRB4*0101/DRA, PRTN3₂₄₁₋₂₅₅-HLA-DRB5*0101/DRA, PRTN3₁₈₃₋₁₉₇-HLA-DRB5*0101/DRA, PRTN3₆₂₋₇₆-HLA-DRB3*0202/DRA, PRTN3₁₁₈₋₁₃₂-HLA-DRB3*0202/DRA, or PRTN3₂₃₉₋₂₅₃-HLA-DRB3*0202/DRA; or
q) Stiff Man Syndrome and the pMHC complex is selected from the group of: GAD₂₁₂₋₂₂₆-HLA-DRB1*0801/DRA, GAD₅₅₅₋₅₆₉-HLA-DRB1*0801/DRA, or GAD₂₉₇₋₃₁₁-HLA-DRB1*0301/DRA.

In some aspects, the pMHC complex is for the treatment of:
a) type I diabetes and the pMHC complex is selected from the group of: PPI_{76-90(K88S)}-HLA-DRB1*0401/DRA, IGRP₁₃₋₂₅-HLA-DRB1*0301/DRA, GAD₅₅₅₋₅₆₇-HLA-DRB1*0401/DRA, GAD_{555-567(557I)}-HLA-DRB1*0401/DRA, IGRP₂₃₋₃₅-HLA-DRB1*0401/DRA, or PPI₇₆₋₉₀-HLA-DRB1*0401/DRA;
b) multiple sclerosis and the pMHC complex is selected from the group of: MBP₈₆₋₉₈-HLA-DRB1*1501/DRA, MBP₈₉₋₁₀₁-HLA-DRB5*0101/DRA, MOG₃₈₋₅₂-HLA-DRB4*0101/DRA, MOG_{97-109(E107S)}-HLA-DRB1*0401/DRA, MOG₂₀₃₋₂₁₇-HLA-DRB3*0101/DRA, PLP₅₄₋₆₈-HLA-DRB3*0101/DRA, PLP₉₄₋₁₀₈-HLA-DRB1*0301/DRA, PLP₂₅₀₋₂₆₄-HLA-DRB4*0101/DRA, MPB₁₃₋₃₂-HLA-DRB5*0101/DRA, MPB₈₃₋₉₉-HLA-DRB5*0101/DRA, MPB₁₁₁₋₁₂₉-HLA-DRB5*0101/DRA, MPB₁₄₆₋₁₇₀-HLA-DRB5*0101/DRA, MOG₂₂₃₋₂₃₇-HLA-DRB3*0202/DRA, MOG₆₋₂₀-HLA-DRB5*0101/DRA, PLP₈₈₋₁₀₂-HLA-DRB3*0202/DRA, or PLP₁₃₉₋₁₅₄-HLA-DRB5*0101/DRA;
c) Celiac Disease and the pMHC complex is selected from the group of: aGlia₅₇₋₆₈-HLA-DQA1*0501/HLA-DQB1*0201, aGlia₆₂₋₇₂-HLA-DQA1*0501/HLA-DQB1*0201, or aGlia₂₁₇₋₂₂₉-HLA-DQA1*0501/HLA-DQB1*0302;
d) primary biliary cirrhosis and the pMHC complex is selected from the group of: PDC-E2₁₂₂₋₁₃₅-HLA-DRB4*0101/DRA, PDC-E2₂₄₉₋₂₆₂-HLA-DRB4*0101/DRA, PDC-E2₂₄₉₋₂₆₃-HLA-DRB1*0801/DRA, PDC-E2₆₂₉₋₆₄₃-HLA-DRB1*0801/DRA, PDC-E2₇₂₋₈₆-HLA-DRB3*0202/DRA, PDC-E2₃₅₃₋₃₆₇-HLA-DRB3*0202/DRA, PDC-E2₄₂₂₋₄₃₆-HLA-DRB3*0202/DRA, PDC-E2₆₂₉₋₆₄₃-HLA-DRB4*0101/DRA, PDC-E2₈₀₋₉₄-HLA-DRB5*0101/DRA, PDC-E2₃₅₃₋₃₆₇-HLA-DRB5*0101/DRA, or PDC-E2₅₃₅₋₅₄₉-HLA-DRB5*0101/DRA;
e) pemphigus folliaceus and/or pemphigus vulgaris and the pMHC complex is selected from the group of: DG1₂₁₆₋₂₂₉-HLA-DRB1*0101/DRA, DG3₉₇₋₁₁₁-HLA-DRB1*0402/DRA, DG3₂₅₁₋₂₆₅-HLA-DRB1*0401/DRA, DG3₄₄₁₋₄₅₅-HLA-DRB1*0402/DRA, DG3₃₅₁₋₃₆₅-HLA-DRB3*0202/DRA, DG3₄₅₃₋₄₆₇-HLA-DRB3*0202/DRA, DG3₅₄₀₋₅₅₄-HLA-DRB3*0202/DRA, DG3₂₈₀₋₂₉₄-HLA-DRB4*0101/DRA, DG3₃₂₆₋₃₄₀-HLA-DRB4*0101/DRA, DG3₃₆₇₋₃₈₁-HLA-DRB4*0101/DRA, DG3₁₃₋₂₇-HLA-DRB5*0101/DRA, DG3₃₂₃₋₃₃₇-HLA-DRB5*0101/DRA, DG3₄₃₈₋₄₅₂-HLA-DRB5*0101/DRA, DG1₄₈₋₆₂-HLA-DRB3*0202/DRA, DG1₂₀₆₋₂₂₂-HLA-DRB3*0202/DRA, DG1₃₆₃₋₃₇₇-HLA-DRB3*0202/DRA, DG1₃₋₁₇-HLA-DRB4*0101/DRA, DG1₁₉₂₋₂₀₆-HLA-DRB4*0101/DRA, DG1₃₂₆₋₃₄₀-HLA-DRB4*0101/DRA, DG1₁₋₁₅-HLA-DRB5*0101/DRA, DG1₃₅₋₄₉-HLA-DRB5*0101/DRA, or DG1₃₂₅₋₃₃₉-HLA-DRB5*0101/DRA;
f) neuromyelitis optica spectrum disorder and the pMHC complex is selected from the group of: AQP4₂₈₄₋₂₉₈-HLA-DRB1*0301/DRA, AQP4₆₃₋₇₆-HLA-DRB1*0301/DRA, AQP4₁₂₉₋₁₄₃-HLA-DRB1*0401/DRA, or AQP4₃₉₋₅₃-HLA-DRB1*1501/DRA;
g) allergic asthma and the pMHC complex is selected from the group of: DERP-1₁₆₋₃₀-HLA-DRB1*0101/DRA, DERP-1₁₆₋₃₀-HLA-DRB1*1501/DRA, DERP1₁₇₁₋₁₈₅-HLA-DRB1*1501/DRA, DERP-1₁₁₀₋₁₂₄-HLA-DPB1*0401/DRA, DERP-2₂₆₋₄₀-HLA-DRB1*0101/DRA; DERP-2₂₆₋₄₀-HLA-DRB1*1501/DRA, or DERP-2₁₀₇₋₁₂₁-HLA-DRB1*0301/DRA;
h) inflammatory bowel disease and the pMHC complex is selected from the group of: bacteroides integrase antigen₁₋₁₅-HLA-DRB5*0101/DRA, bacteroides integrase antigen₁₈₃₋₁₉₇-HLA-DRB3*0101/DRA, bacteroides integrase antigen₇₀₋₈₄-HLA-DRB4*0101/DRA, bacteroides integrase antigen₄₋₁₈-HLA-DRB3*0202/DRA, bacteroides integrase antigen₁₇₁₋₁₈₅-HLA-DRB3*0202/DRA, bacteroides integrase antigen₂₅₆₋₂₇₀-HLA-DRB3*0202/DRA, Fla-2/Fla-X₃₆₆₋₃₈₀-HLA-DRB3*0101/DRA, Fla-2/Fla-X₂₆₁₋₂₇₅-HLA-DRB5*0101/DRA, Fla-2/Fla-X₅₁₋₆₅-HLA-DRB4*0101/DRA, Fla-2/Fla-X₄₋₁₈-HLA-DRB3*0202/DRA, Fla-2/Fla-X₂₆₁₋₂₇₅-HLA-DRB3*0202/DRA, Fla-2/Fla-X₂₇₁₋₂₈₅-HLA-DRB3*0202/DRA, YIDX₇₈₋₉₂-HLA-DRB3*0101/DRA, YIDX₇₈₋₉₂-HLA-DRB4*0101/DRA, YIDX₉₈₋₁₁₂-HLA-DRB5*0101/DRA, YIDX₂₂₋₃₆-HLA-DRB3*0202/DRA, YIDX₈₀₋₉₄-HLA-DRB3*0202/DRA, or YIDX₁₀₁₋₁₁₅-HLA-DRB3*0202/DRA;
i) emphysema and the pMHC complex is selected from the group of: elastin₈₉₋₁₀₃-HLA-DRB3*0101/DRA, elastin₆₉₈₋₇₁₂-HLA-DRB5*0101/DRA, elastin₅₅₈₋₅₇₂-HLA-DRB4*0101/DRA, elastin₅₆₆₋₅₈₀-HLA-DRB3*0202/DRA, or elastin₅₄₅₋₆₅₉-HLA-DRB3*0202/DRA;
j) psoriasis and the pMHC complex is selected from the group of: Cap18₆₄₋₇₈-HLA-DRB3*0101/DRA, Cap18₃₄₋₄₈-HLA-DRB3*0101/DRA, Cap18₄₇₋₆₁-HLA-DRB3*0101/DRA, Cap18₁₅₁₋₁₆₅-HLA-DRB4*0101/DRA, Cap18₁₄₉₋₁₆₃-HLA-DRB5*0101/DRA, Cap18₁₅₂₋₁₆₆-HLA-DRB5*0101/DRA, Cap18₁₃₁₋₁₄₅-HLA-DRB5*0101/DRA, Cap18₂₄₋₃₈-HLA-DRB3*0202/DRA, ADMTSL5₂₄₅₋₂₅₉-HLA-DRB3*0101/DRA, ADMTSL5₂₆₇₋₂₈₁-HLA-DRB3*0101/DRA, ADMTSL5₃₇₂₋₃₈₆-HLA-DRB3*0101/DRA, ADMTSL5₂₈₉₋₃₀₃-HLA-DRB4*0101/DRA, ADMTSL5₃₉₆₋₄₁₀-HLA-DRB4*0101/DRA, ADMTSL5₄₃₃₋₄₄₇-HLA-DRB4*0101/DRA, ADMTSL5₁₄₂₋₁₅₆-HLA-DRB5*0101/DRA, ADMTSL5₂₃₆₋₂₅₀-HLA-DRB5*0101/DRA, ADMTSL5₃₀₁₋₃₁₅-HLA-DRB5*0101/DRA, ADMTSL5₂₀₃₋₂₁₇-HLA-DRB3*0202/DRA, ADMTSL5₄₀₄₋₄₁₈-HLA-DRB3*0202/DRA, or ADMTSL5₄₃₃₋₄₄₇-HLA-DRB3*0202/DRA;
k) autoimmune hepatitis and the pMHC complex is selected from the group of: CYP2D6₁₉₃₋₂₀₇-HLA-DRB1*0301/DRA, CYP2D6₇₆₋₉₀-HLA-DRB1*0301/DRA, CYP2D6₂₉₃₋₃₀₇-HLA-DRB1*0301/DRA, CYP2D6₃₁₃₋₃₃₂-HLA-DRB1*0301/DRA, CYP2D6₃₉₃₋₄₁₂-HLA-DRB1*0301/DRA, CYP2D6₁₉₉₋₂₁₃-HLA-DRB1*0401/DRA, CYP2D6₄₅₀₋₄₆₄-HLA-DRB1*0401/DRA, CYP2D6₃₀₁₋₃₁₅-HLA-DRB1*0401/DRA, CYP2D6₄₅₂₋₄₆₆-HLA-DRB1*0701/DRA, CYP2D6₅₉₋₇₃-HLA-DRB1*0701/DRA, CYP2D6₁₃₀₋₁₄₄-HLA-DRB1*0701/DRA, CYP2D6₁₉₃₋₂₁₂-HLA-DRB1*0701/DRA, CYP2D6₃₀₅₋₃₂₄-HLA-DRB1*0701/DRA, CYP2D6₁₃₁₋₁₄₅-HLA-DRB3*0202/DRA, CYP2D6₂₁₆₋₂₃₀-HLA-DRB3*0202/DRA, CYP2D6₂₃₈₋₂₅₂-HLA-DRB3*0202/DRA, CYP2D6₁₉₉₋₂₁₃-HLA-DRB4*0101/DRA, CYP2D6₂₃₅₋₂₅₂-HLA-DRB4*0101/DRA, CYP2D6₂₉₃₋₃₀₇-HLA-DRB4*0101/DRA, CYP2D6₂₃₈₋₂₅₂-HLA-DRB5*0101/DRA, CYP2D6₃₈₁₋₃₉₅-HLA-DRB5*0101/DRA, CYP2D6₄₂₉₋₄₄₃-HLA-DRB5*0101/DRA, SLA₃₃₄₋₃₄₈-HLA-DRB1*0301/DRA, SLA₁₉₆₋₂₁₀-HLA-DRB1*0301/DRA, SLA₁₁₅₋₁₂₉-HLA-DRB1*0301/DRA, SLA₃₇₃₋₃₈₆-HLA-DRB1*0301/DRA, SLA₁₈₆₋₁₉₇-HLA-DRB1*0301/DRA, SLA₃₁₇₋₃₃₁-HLA-DRB1*0401/DRA, SLA₁₇₁₋₁₈₅-HLA-DRB1*0401/DRA, SLA₄₁₇₋₄₃₁-HLA-DRB1*0401/DRA, SLA₃₅₉₋₃₇₃-HLA-DRB1*0701/DRA, SLA₂₁₅₋₂₂₉-HLA-DRB1*0701/DRA, SLA₁₁₁₋₁₂₅-HLA-DRB1*0701/DRA, SLA₁₁₀₋₁₂₄-HLA-DRB3*0202/DRA, SLA₂₉₉₋₃₁₃-HLA-DRB3*0202/DRA, SLA₃₄₂₋₃₅₆-HLA-DRB3*0202/DRA, SLA₄₉₋₆₃-HLA-DRB4*0101/DRA, SLA₁₁₉₋₁₃₃-HLA-DRB4*0101/DRA, SLA₂₆₀₋₂₇₄-HLA-DRB4*0101/DRA, SLA₂₆₋₄₀-HLA-DRB5*0101/DRA, SLA₈₆₋₁₀₀-HLA-DRB5*0101/DRA, or SLA₃₃₁₋₃₄₅-HLA-DRB5*0101/DRA;
l) uveitis and the pMHC complex is selected from the group of: arrestin₁₉₉₋₂₁₃-HLA-DRB3*0101/DRA, arrestin₇₇₋₉₁-HLA-DRB3*0101/DRA, arrestin₂₅₀₋₂₆₄-HLA-DRB3*0101/DRA, arrestin₁₇₂₋₁₈₆-HLA-DRB4*0101/DRA, arrestin₃₅₄₋₃₆₈-HLA-DRB4*0101/DRA, arrestin₂₃₉₋₂₅₃-HLA-DRB4*0101/DRA, arrestin₁₀₂₋₁₁₆-HLA-DRB5*0101/DRA, arrestin₅₉₋₇₃-HLA-DRB5*0101, arrestin₂₈₀₋₂₉₄-HLA-DRB5*0101, arrestin₂₉₁₋₃₀₆-HLA-DRB1*0301/DRA, arrestin₁₉₅₋₂₀₉-HLA-DRB3*0202/DRA, arrestin₁₉₉₋₂₁₃-HLA-DRB3*0202/DRA, or arrestin₂₀₀₋₂₁₄-HLA-DRB3*0202/DRA;
m) Sjogren Syndrome and the pMHC complex is selected from the group of: RO60₁₂₇₋₁₄₁-HLA-DRB1*0301/DRA, RO60₅₂₃₋₅₃₇-HLA-DRB1*0301/DRA, RO60₂₄₃₋₂₅₇-HLA-DRB1*0301/DRA, RO60₄₈₄₋₄₉₈-HLA-DRB3*0101/DRA, RO60₃₄₇₋₃₆₁-HLA-DRB3*0101/DRA, RO60₃₆₉₋₃₈₃-HLA-DRB3*0101/DRA, RO60₄₂₆₋₄₄₀-HLA-DRB4*0101/DRA, RO60₂₆₇₋₂₈₁-HLA-DRB4*0101/DRA, RO60₁₇₈₋₁₉₂-HLA-DRB4*0101/DRA, RO60₃₅₈₋₃₇₂-HLA-DRB5*0101/DRA, RO60₂₂₁₋₂₃₅-HLA-DRB5*0101/DRA, RO60₃₁₈₋₃₃₂-HLA-DRB5*0101/DRA, RO60₅₁₋₆₅-HLA-DRB3*0202/DRA, RO60₃₁₂₋₃₂₆-HLA-DRB3*0202/DRA, RO60₃₄₇₋₃₆₁-HLA-DRB3*0202/DRA, LA₂₄₁₋₂₅₅-HLA-DRB1*0301/DRA, LA₁₀₁₋₁₁₅-HLA-DRB1*0301/DRA, LA₁₅₃₋₁₆₇-HLA-DRB1*0301/DRA, LA₁₇₈₋₁₉₂-HLA-DRB3*0101/DRA, LA₁₉₋₃₃-HLA-DRB3*0101/DRA, LA₃₇₋₅₁-HLA-DRB3*0101/DRA, LA₁₃₃₋₁₄₇-HLA-DRB4*0101/DRA, LA₅₀₋₆₄-HLA-DRB4*0101/DRA, LA₃₂₋₄₆-HLA-DRB4*0101/DRA, LA₁₅₃₋₁₆₇-HLA-DRB5*0101/DRA, LA₈₃₋₉₇-HLA-DRB5*0101/DRA, LA₁₃₆₋₁₅₀-HLA-DRB5*0101/DRA, LA₅₀₋₆₄-HLA-DRB3*0202/DRA, LA₈₆₋₁₀₀-HLA-DRB3*0202/DRA, or LA₁₅₄₋₁₆₈-HLA-DRB3*0202/DRA;
n) scleroderma and the pMHC complex is selected from the group of: TOP1₃₄₆₋₃₆₀-HLA-DRB3*0101/DRA, TOP1₄₂₀₋₄₃₄-HLA-DRB3*0101/DRA, TOP1₇₅₀₋₇₆₄-HLA-DRB3*0101/DRA, TOP1₄₁₉₋₄₃₃-HLA-DRB4*0101/DRA, TOP1₅₉₁₋₆₀₅-HLA-DRB4*0101/DRA, TOP1₆₉₅₋₇₀₉-HLA-DRB4*0101/DRA, TOP1₃₀₅₋₃₁₉-HLA-DRB5*0101/DRA, TOP1₃₄₆₋₃₆₀-HLA-DRB5*0101/DRA, TOP1₄₁₉₋₄₃₃-HLA-DRB5*0101/DRA, TOP1₄₂₀₋₄₃₄-HLA-DRB3*0202/DRA, TOP1₄₂₅₋₄₃₉-HLA-DRB3*0202/DRA, TOP1₆₁₄₋₆₂₈-HLA-DRB3*0202/DRA, CENP-C₂₉₇₋₃₁₁-HLA-DRB3*0101/DRA, CENP-C₈₅₇₋₈₇₁-HLA-DRB3*0101, CENP-C₈₈₇₋₉₀₁-HLA-DRB3*0101, CENP-C₂₁₂₋₂₂₆-HLA-DRB4*0101/DRA, CENP-C₆₄₃₋₆₅₇-HLA-DRB4*0101/DRA, CENP-C₈₃₂₋₅₄₆-HLA-DRB4*0101/DRA, CENP-C₁₆₇₋₁₈₁-HLA-DRB5*0101/DRA, CENP-C₂₄₆₋₂₆₀-HLA-DRB5*0101/DRA, CENP-C₈₄₆₋₈₆₀-HLA-DRB5*0101/DRA, CENP-C₁₄₉₋₁₆₃-HLA-DRB3*0202/DRA, CENP-C₈₃₃₋₈₄₇-HLA-DRB3*0202/DRA, or CENP-C₈₄₇₋₈₆₁-HLA-DRB3*0202/DRA;
o) anti-phospholipid syndrome and the pMHC complex is selected from the group of: APOH₂₃₅₋₂₄₉-HLA-DRB3*0101/DRA, APOH₃₀₆₋₃₂₀-HLA-DRB3*0101/DRA, APOH₂₃₇₋₂₅₁-HLA-DRB3*0101/DRA, APOH₂₉₅₋₃₀₉-HLA-DRB3*0101/DRA, APOH₂₈₋₄₂-HLA-DRB4*0101/DRA, APOH₁₇₃₋₁₈₇-HLA-DRB4*0101/DRA, APOH₂₆₄₋₂₇₈-HLA-DRB4*0101/DRA, APOH₂₉₅₋₃₀₉-HLA-DRB4*0101/DRA, APOH₄₉₋₆₃-HLA-DRB5*0101/DRA, APOH₂₆₉₋₂₈₃-HLA-DRB5*0101/DRA, APOH₂₉₅₋₃₀₉-HLA-DRB5*0101/DRA, APOH₃₂₁₋₃₅₅-HLA-DRB3*0202/DRA, APOH₃₂₂₋₃₃₆-HLA-DRB3*0202/DRA, or APOH₃₂₄₋₃₃₈-HLA-DRB3*0202/DRA;
p) ANCA-associated vasculitis and the pMHC complex is selected from the group of: MPO₅₀₆₋₅₂₀-HLA-DRB3*0101/DRA, MPO₃₀₂₋₃₁₆-HLA-DRB3*0101/DRA, MPO₇₋₂₁-HLA-DRB3*0101/DRA, MPO₆₈₉₋₇₀₃-HLA-DRB4*0101/DRA, MPO₂₄₈₋₂₆₂-HLA-DRB4*0101/DRA, MPO₄₄₄₋₄₅₈-HLA-DRB4*0101/DRA, MPO₅₁₃₋₅₂₇-HLA-DRB5*0101/DRA, MPO₉₇₋₁₁₁-HLA-DRB5*0101/DRA, MPO₆₁₆₋₆₃₀-HLA-DRB5*0101/DRA, MPO₄₆₂₋₄₇₆-HLA-DRB3*0202/DRA, MPO₆₁₇₋₆₃₁-HLA-DRB3*0202/DRA, MPO₇₁₄₋₇₂₈-HLA-DRB3*0202/DRA, PRTN3₄₄₋₅₈-HLA-DRB3*0101/DRA, PRTN3₂₃₄₋₂₄₈-HLA-DRB3*0101/DRA, PRTN3₅₉₋₇₃-HLA DRB3*0101/DRA, PRTN3₅₉₋₇₃-HLA-DRB5*0101/DRA, PRTN3₁₁₇₋₁₃₁-HLA-DRB4*0101/DRA, PRTN3₁₆₄₋₁₇₈-HLA-DRB4*0101/DRA, PRTN3₇₁₋₈₅-HLA-DRB4*0101/DRA, PRTN3₂₄₁₋₂₅₅-HLA-DRB5*0101/DRA, PRTN3₁₈₃₋₁₉₇-HLA-DRB5*0101/DRA, PRTN3₆₂₋₇₆-HLA-DRB3*0202/DRA, PRTN3₁₁₈₋₁₃₂-HLA-DRB3*0202/DRA, or PRTN3₂₃₉₋₂₅₃-HLA-DRB3*0202/DRA; or
q) Stiff Man Syndrome and the pMHC complex is selected from the group of: GAD₂₁₂₋₂₂₆-HLA-DRB1*0801/DRA, GAD₅₅₅₋₅₆₉-HLA-DRB1*0801/DRA, or GAD₂₉₇₋₃₁₁-HLA-DRB1*0301/DRA.

Selection of the co-stimulatory molecule or molecules to be coupled to the pMHC/NP complex may also be similarly optimized and will largely depend on the nature of the immune cell population in need of differentiation or expansion. For instance, if the intent is to expand or differentiate T regulatory cell populations, relevant combinations may include, but are not limited to, co-stimulatory molecules and cytokines such as IL15-IL15Ra, IL-2, IL-10, IL-35, ICOS-L, IL2/Anti-IL2 mAb complex, TGF-beta, IL-21, ITE or ICOSL. In contrast, in certain embodiments, such as with certain types of cancers, an expansion and/or differentiation of the T regulatory phenotype may not be the desired response. Thus, alternative co-stimulatory molecules and cytokines would be optimized to the particular treatment.

### Methods of Making Nanoparticles and Complexes

MHCs and nanoparticles can be made by a variety of methods. The following are merely exemplary.

### MHCs

To make MHC class I complexes, two exemplary methods are provided. The first involves re-folding MHC class I heavy and light chains, which are expressed in bacteria in the presence of peptide, followed by purification via gel filtration and anion exchange chromatography, as described in the literature (Garboczi, D. N. et al. (1992) Proc Natl. Acad Sci USA 89:3429-3433; Altman, J. D. et al. (1996) Science 274:94-96). The second involves expressing MHC class I complexes at high yields in lentiviral-transduced freestyle CHO cells as single chain constructs in which the peptide-coding sequence, the MHC class I light and heavy chains are sequentially tethered with flexible GS linkers (Yu, Y. Y. et al. (2002) J Immunol 168:3145-3149) followed by a carboxyterminal linker encoding a BirA site, a 6×His tag ending with a free Cys. The secreted proteins are purified from culture supernatants using nickel columns and anion exchange chromatography and are used directly for NP coating or are biotinylated to produce pMHC tetramers using fluorochrome-conjugated streptavidin. Tetramers generated using representative single-chain pMHC complexes encoding the IGRP₂₀₆₋₂₁₄ autoantigenic peptide or its mimic NRP-V7 efficiently bind to cognate monoclonal autoreactive CD8⁺ T-cells but not to their polyclonal counterparts as determined by flow cytometry.

Recombinant pMHC class II monomers can be purified from Drosophila SC2 cells transfected with constructs encoding I-Aβ and I-Aα chains carrying c-Jun or c-Fos leucine zippers, respectively, and a BirA and 6×His tags as previously described (Stratmann, T. et al. (2000) J Immunol 165:3214-3225, Stratmann, T. et al. (2003) J. Clin. Invest. 112:3214-3225). As the yields of this approach are generally low and time-consuming, Applicant has developed an expression system in freestyle CHO cells transduced with lentiviruses encoding a monocistronic message in which the peptide-IAβ and α chains of the complex are separated by the ribosome skipping P2A sequence (Holst, J. et al. (2006) Nat Protoc 1:406-417). As with the single chain pMHC class I constructs described above, a linker encoding a BirA site, a 6×His tag and a free Cys is added to the carboxyterminal end of the construct. The self-assembled pMHC class II complexes are purified from the cell culture supernatants by nickel chromatography followed by anion exchange and are used for coating onto NPs or are processed for biotinylation and tetramer formation as described above. pMHC class II tetramers generated using a representative pMHC class II complex encoding the 2.5mi autoantigenic peptide are specifically and efficiently bound by cognate monoclonal autoreactive CD4+ T-cells, as determined by flow cytometry.

PE-conjugated tetramers can be prepared using biotinylated pMHC monomers as described (Stratmann, T. et al. (2000) J Immunol 165:3214-3225; Stratmann, T. et al. (2003) J. Clin. Invest. 112:3214-3225; Amrani, A. et al. (2000) Nature 406:739-742). Peripheral blood mononuclear cells, splenocytes and lymph node CD8+ or CD4+ T-cells can be stained with tetramer (5 ug/mL) in FACS buffer (0.1% sodium azide and 1% FBS in PBS) for 1 h at 4° C., washed, and incubated with FITC-conjugated anti-CD8u or anti-CD4 (5 μg/mL) and PerCP-conjugated anti-B220 (2 μg/mL; as a ‘dumb’ gate) for 30 min at 4° C. Cells are washed, fixed in 1% PFA/PBS and analyzed by FACS.

### NP Synthesis

Nanoparticles may be formed by contacting an aqueous phase containing the co-stimulatory molecule(s), the pMHC complex and/or cytokine, and a polymer and a nonaqueous phase followed by evaporation of the nonaqueous phase to cause the coalescence of particles from the aqueous phase as taught in U.S. Pat. No. 4,589,330 or 4,818,542. Certain polymers for such preparations are natural or synthetic copolymers or polymers which include gelatin agar, starch, arabinogalactan, albumin, collagen, polyglycolic acid, polylactic acid, glycolide-L(−) lactide poly(episilon-caprolactone, poly(epsilon-caprolactone-CO-lactic acid), poly(epsilon-caprolactone-CO-glycolic acid), poly(β-hydroxy butyric acid), poly(ethylene oxide), polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly, certain polymers are polyesters, such as polyglycolic acid, polylactic acid, glycolide-L(−) lactide poly(episilon-caprolactone), poly(epsilon-caprolactone-CO-lactic acid), and poly(epsilon-caprolactone-CO-glycolic acid). Solvents useful for dissolving the polymer include: water, hexafluoroisopropanol, methylenechloride, tetrahydrofuran, hexane, benzene, or hexafluoroacetone sesquihydrate.

Gold nanoparticles (GNPs) are synthesized using chemical reduction of gold chloride with sodium citrate as described (Perrault, S. D. et al. (2009) Nano Lett 9:1909-1915). Briefly, 2 mL of 1% of HAuCl₄ (Sigma Aldrich) is added to 100 mL H₂O under vigorous stirring and the solution is heated in an oil bath. Six (for 14 nm GNPs) or two mL (for 40 nm GNPs) of 1% Na Citrate is added to the boiling HAuCl₄ solution, which is stirred for an additional 10 min and then is cooled down to room temperature. GNPs are stabilized by the addition of 1 μMol of thiol-PEG linkers (Nanocs, MA) functionalized with —COOH or —NH₂ groups as acceptors of MHC. Pegylated GNPs are washed with water to remove free thiol-PEG, concentrated and stored in water for further analysis. NP density is determined via spectrophotometry and calculated according to Beer's law.

The SFP series iron oxide NPs (SFP IONPs) can also be produced by thermal decomposition of iron acetate in organic solvents in the presence of surfactants, then rendered solvent in aqueous buffers by pegylation (Xie, J. et al. (2007) Adv Mater 19:3163; Xie, J. et al. (2006) Pure Appl. Chem. 78:1003-1014; Xu, C. et al. (2007) Polymer International 56:821-826). Briefly, 2 mMol Fe(acac)₃ (Sigma Aldrich, Oakville, ON) are dissolved in a mixture of 10 mL benzyl ether and oleylamine and heated to 100° C. for 1 hr followed by 300° C. for 2 hr with reflux under the protection of a nitrogen blanket. Synthesized NPs are precipitated by addition of ethanol and resuspended in hexane. For pegylation of the IONPs, 100 mg of different 3.5 kDa DPA-PEG linkers (Jenkem Tech USA) are dissolved in a mixture of CHCl₃ and HCON(CH₃)₂ (dimethylformamide (DMF)). The NP solution (20 mg Fe) is then added to the DPA-PEG solution and stirred for 4 hr at room temperature. Pegylated SFP NPs are precipitated overnight by addition of hexane and then resuspended in water. Trace amounts of aggregates are removed by high-speed centrifugation (20,000×g, 30 min), and the monodisperse SFP NPs are stored in water for further characterization and pMHC conjugation. The concentration of iron in IONP products is determined by spectrophotometry at A410 in 2N HCL. Based on the molecular structure and diameter of SFP NPs (Fe₃O₄; 8±1 nm diameter) (Xie, J. et al. (2007) Adv Mater 19:3163; Xie, J. et al. (2006) Pure Appl. Chem. 78:1003-1014), Applicant estimates that SFP solutions containing 1 mg of iron contain 5×10¹⁴ NPs.

The nanoparticles can also be made by thermally decomposing or heating a nanoparticle precursor. In one embodiment, the nanoparticle is a metal or a metal oxide nanoparticle. In one embodiment, the nanoparticle is an iron oxide nanoparticle. In one embodiment, the nanoparticle is a gold nanoparticle. In one embodiment, provided herein are the nanoparticles prepared in accordance with the present technology. In one embodiment, provided herein is a method of making iron oxide nanoparticles comprising a thermal decomposition reaction of iron acetyl acetonate. In one embodiment, the iron oxide nanoparticle obtained is water-soluble. In one aspect, the iron oxide nanoparticle is suitable for protein conjugation. In one embodiment, the method comprises a single-step thermal decomposition reaction.

In one aspect, the thermal decomposition occurs in the presence of functionalized PEG molecules. Certain non-limiting examples of functionalized PEG linkers are shown in Table 1.

In one aspect, the thermal decomposition comprises heating iron acetyl acetonate. In one embodiment, the thermal decomposition comprises heating iron acetyl acetonate in the presence of functionalized PEG molecules. In one embodiment, the thermal decomposition comprises heating iron acetyl acetonate in the presence of benzyl ether and functionalized PEG molecules.

Without being bound by theory, in one embodiment, functionalized PEG molecules are used as reducing reagents and as surfactants. The method of making nanoparticles provided herein simplifies and improves conventional methods, which use surfactants that are difficult to be displaced, or are not displaced to completion, by PEG molecules to render the particles water-soluble. Conventionally, surfactants can be expensive (e.g., phospholipids) or toxic (e.g., Oleic acid or oleilamine). In another aspect, without being bound by theory, the method of making nanoparticles obviates the need to use conventional surfactants, thereby achieving a high degree of molecular purity and water solubility.

In one embodiment, the thermal decomposition involves iron acetyl acetonate and benzyl ether and in the absence of conventional surfactants other than those employed herein.

In one embodiment, the temperature for the thermal decomposition is about 80° C. to about 300° C., or about 80° C. to about 200° C., or about 80° C. to about 150° C., or about 100° C. to about 250° C., or about 100° C. to about 200° C., or about 150° C. to about 250° C., or about 150° C. to about 250° C. In one embodiment, the thermal decomposition occurs at about 1 to about 2 hours of time.

In one embodiment, the method of making the iron oxide nanoparticles comprises a purification step, such as by using Miltenyi Biotec LS magnet column.

In one embodiment, the nanoparticles are stable at about 4° C. in phosphate buffered saline (PBS) without any detectable degradation or aggregation. In one embodiment, the nanoparticles are stable for at least 6 months.

In one aspect, provided herein is a method of making nanoparticle complexes comprising contacting pMHC with iron oxide nanoparticles provided herein. Without being bound by theory, pMHC encodes a Cysteine at its carboxyterminal end, which can react with the maleimide group in functionalized PEG at about pH 6.2 to about pH 6.5 for about 12 to about 14 hours.

In one aspect, the method of making nanoparticle complexes comprises a purification step, such as by using Miltenyi Biotec LS magnet column. Coupling to Nanoparticles

In certain aspects, antigen-MHC complex and/or cytokine and/or costimulatory molecule can be coupled to the nanoparticle core by one or more of covalently, non-covalently, or cross-linked and optionally coupled through a linker. In further aspects, the linker may be less than 5 kD in size, and is optionally polyethylene glycol. In aspects involving a linker or linkers, the linkers may be the same or different from each other on a single nanoparticle core.

In order to couple the substrate or particles to the antigen-MHC complex and/or cytokine and/or costimulatory molecule, the following techniques can be applied.

The binding can be generated by chemically modifying the substrate or particle which typically involves the generation of “functional groups” on the surface, said functional groups being capable of binding to an MHC complex, and/or linking the optionally chemically modified surface of the surface or particle with covalently or non-covalently bound so-called “linking molecules,” followed by reacting the MHC or MHC complex with the particles obtained.

The term “linking molecule” or “linker” means a substance capable of linking with the substrate or particle and also capable of linking to an MHC complex.

The term “functional groups” as used hereinbefore is not restricted to reactive chemical groups forming covalent bonds, but also includes chemical groups leading to an ionic interaction or hydrogen bonds with the MHC complex. Moreover, it should be noted that a strict distinction between “functional groups” generated at the surface and linking molecules bearing “functional groups” is not possible, since sometimes the modification of the surface requires the reaction of smaller linking molecules such as ethylene glycol with the particle surface.

The functional groups or the linking molecules bearing them may be selected from amino groups, carbonic acid groups, thiols, thioethers, disulfides, guanidino, hydroxyl groups, amine groups, vicinal diols, aldehydes, alpha-haloacetyl groups, mercury organyles, ester groups, acid halide, acid thioester, acid anhydride, isocyanates, isothiocyanates, sulfonic acid halides, imidoesters, diazoacetates, diazonium salts, 1,2-diketones, phosphonic acids, phosphoric acid esters, sulfonic acids, azolides, imidazoles, indoles, N-maleimides, alpha-beta-unsaturated carbonyl compounds, arylhalogenides or their derivatives.

Non-limiting examples for other linking molecules with higher molecular weights are nucleic acid molecules, polymers, copolymers, polymerizable coupling agents, silica, proteins, and chain-like molecules having a surface with the opposed polarity with respect to the substrate or particle. Nucleic acids can provide a link to affinity molecules containing themselves nucleic acid molecules, though with a complementary sequence with respect to the linking molecule.

In some embodiments, the linking molecule comprises polyethylene glycol. In some embodiments, the linking molecule comprises polyethylene glycol and maleimide. In some embodiments, the polyethylene glycol comprises one or more of a C₁-C₃ alkoxy group, —R¹⁰NHC(O)R—, —R¹⁰C(O)NHR—, —R¹⁰OC(O)R—, —R¹⁰C(O)OR—, wherein each R is independently H or C₁-C₆ alkyl and wherein each R¹⁰ is independently a bond or C₁-C₆ alkyl.

As examples for polymerizable coupling agents, diacetylene, styrene butadiene, vinylacetate, acrylate, acrylamide, vinyl compounds, styrene, silicone oxide, boron oxide, phosphorous oxide, borates, pyrrole, polypyrrole and phosphates can be cited.

pMHC complexes can be coupled to nanoparticles by a variety of methods, one non-limiting example includes conjugation to NPs produced with PEG linkers carrying distal —NH₂ or —COOH groups that can be achieved via the formation of amide bonds in the presence of 1-Ethyl-3-[3-dimethylaminopropyl]carbodiimide hydrochloride (EDC). NPs with —COOH groups are first dissolved in 20 mM MES buffer, pH 5.5. N-hydroxysulfosuccinimide sodium salt (sulpha-NHS, Thermo scientific, Waltham, MA, final concentration 10 mM) and EDC (Thermo scientific, Waltham, MA, final concentration 1 mM) is added to the NP solution. After 20 min of stirring at room temperature, the NP solution is added drop-wise to the solution containing pMHC monomers dissolved in 20 mM borate buffer (pH 8.2). The mixture is stirred for an additional 4 hr. To conjugate MHCs to NH₂-functionalized NPs pMHC complexes are first dissolved in 20 mM MES buffer, pH 5.5, containing 100 mM NaCl. Sulpha-NHS (10 mM) and EDC (5 mM) are then added to the MHC solution. The activated MHC molecules are then added to the NP solution in 20 mM borate buffer (pH 8.2), and stirred for 4 hr at room temperature.

To conjugate MHC to maleimide-functionalized NPs, pMHC complexes are first incubated with Tributylphospine (TBP, 1 mM) for 4 hr at room temperature. pMHCs engineered to encode a free carboxyterminal Cys residue are then mixed with NPs in 40 mM phosphate buffer, pH 6.0, containing 2 mM EDTA, 150 mM NaCl, and incubated overnight at room temperature. MHCs of the pMHC complexes are covalently bound with NPs via the formation of a carbon-sulfide bond between meleimide groups and the Cys residue.

Click chemistry can be used to conjugate pMHC or avidin to NPs functionalized with azide groups. For this reaction, MHC or avidin molecules are first incubated with dibenzocyclooctyl (DBCO, Click Chemistry Tools, Scottdale, AZ) reagent for 2 hr at room temperature. Free DBCO molecules can be removed by dialysis overnight. MHC- or avidin-DBCO conjugates are then incubated with SFP-Z for 2 hr, resulting in formation of triazole bonds between pMHCs or avidin molecules and NPs.

Unconjugated pMHC complexes in the different MHC-NP conjugating reactions can be removed by extensive dialysis using methods known in the art. A non-limiting example is dialysis against PBS, pH 7.4, at 4° C. though 300 kDa molecular weight cut off membranes (Spectrum labs). Alternatively, pMHC-conjugated IONPs can be purified by magnetic separation. The conjugated NPs can be concentrated by ultrafiltration through Amicon Ultra-15 units (100 kDa MWCO) and stored in PBS.

The surface of the substrate or particle can be chemically modified, for instance by the binding of phosphonic acid derivatives having functional reactive groups. One example of these phosphonic acid or phosphonic acid ester derivates is imino-bis(methylenphosphono) carbonic acid which can be synthesized according to the “Mannich-Moedritzer” reaction. This binding reaction can be performed with a substrate or a particle as directly obtained from the preparation process or after a pre-treatment (for instance with trimethylsilyl bromide). In the first case the phophonic acid (ester) derivative may for instance displace components of the reaction medium which are still bound to the surface. This displacement can be enhanced at higher temperatures. Trimethylsilyl bromide, on the other hand, is believed to dealkylate alkyl group-containing phosphorous-based complexing agents, thereby creating new binding sites for the phosphonic acid (ester) derivative. The phosphonic acid (ester) derivative, or linking molecules bound thereto, may display the same functional groups as given above. A further example of the surface treatment of the substrate or particle involves heating in a diol such as ethylene glycol. It should be noted that this treatment may be redundant if the synthesis already proceeded in a diol. Under these circumstances the synthesis product directly obtained is likely to show the necessary functional groups. This treatment is, however, applicable to a substrate or a particle that was produced in N- or P-containing complexing agents. If such substrate or particle is subjected to an after-treatment with ethylene glycol, ingredients of the reaction medium (e.g. complexing agent) still binding to the surface can be replaced by the diol and/or can be dealkylated.

It is also possible to replace N-containing complexing agents still bound to the particle surface by primary amine derivatives having a second functional group. The surface of the substrate or particle can also be coated with silica. Silica allows a relatively simple chemical conjugation of organic molecules since silica easily reacts with organic linkers, such as triethoxysilane or chlorosilane. The particle surface may also be coated by homo- or copolymers. Examples for polymerizable coupling agents are: N-(3-aminopropyl)-3-mercaptobenzamidine, 3-(trimethoxysilyl)propylhydrazide and 3-trimethoxysilyl)propylmaleimide. Other non-limiting examples of polymerizable coupling agents are mentioned herein. These coupling agents can be used singly or in combination depending on the type of copolymer to be generated as a coating.

Another surface modification technique that can be used with substrates or particles containing oxidic transition metal compounds is conversion of the oxidic transition metal compounds by chlorine gas or organic chlorination agents to the corresponding oxychlorides. These oxychlorides are capable of reacting with nucleophiles, such as hydroxy or amino groups as often found in biomolecules. This technique allows generating a direct conjugation with proteins, for instance, via the amino group of lysine side chains. The conjugation with proteins after surface modification with oxychlorides can also be effected by using a bi-functional linker, such as maleimidopropionic acid hydrazide.

For non-covalent linking techniques, chain-type molecules having a polarity or charge opposite to that of the substrate or particle surface are particularly suitable. Examples for linking molecules which can be non-covalently linked to core/shell nanoparticles involve anionic, cationic or zwitter-ionic surfactants, acid or basic proteins, polyamines, polyamides, polysulfone or polycarboxylic acid. The hydrophobic interaction between substrate or particle and amphiphilic reagent having a functional reactive group can generate the necessary link. In particular, chain-type molecules with amphiphilic character, such as phospholipids or derivatised polysaccharides, which can be crosslinked with each other, are useful. The absorption of these molecules on the surface can be achieved by coincubation. The binding between affinity molecule and substrate or particle can also be based on non-covalent, self-organizing bonds. One example thereof involves simple detection probes with biotin as linking molecule and avidin- or strepdavidin-coupled molecules.

Protocols for coupling reactions of functional groups to biological molecules can be found in the literature, for instance in “Bioconjugate Techniques” (Greg T. Hermanson, Academic Press 1996). The biological molecule (e.g., MHC molecule or derivative thereof) can be coupled to the linking molecule, covalently or non-covalently, in line with standard procedures of organic chemistry such as oxidation, halogenation, alkylation, acylation, addition, substitution or amidation. These methods for coupling the covalently or non-covalently bound linking molecule can be applied prior to the coupling of the linking molecule to the substrate or particle or thereafter. Further, it is possible, by means of incubation, to effect a direct binding of molecules to correspondingly pre-treated substrate or particles (for instance by trimethylsilyl bromide), which display a modified surface due to this pre-treatment (for instance a higher charge or polar surface).

### Pharmaceutical Compositions and Administration

Provided herein are pharmaceutical compositions useful for the treatment and prevention of disease. The compositions comprise, or alternatively consist essentially of, or yet further consist of, a nanoparticle complex as described herein and a carrier.

The compositions can be used to induce or modify an immune response against a disease relevant antigen, e.g., a polypeptide, a peptide, a carbohydrate, a lipid or other molecule or molecular fragment and against developing a condition or disease caused by such an autoimmune response or cancer.

Compositions of the disclosure may be conventionally administered parenterally, by injection, for example, intravenously, subcutaneously, or intramuscularly. Additional formulations which are suitable for other modes of administration include oral formulations. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%. The preparation of an aqueous composition that contains an antigen-MHC-nanoparticle complex that modifies the subject's immune condition will be known to those of skill in the art in light of the present disclosure. In certain embodiments, a composition may be inhaled (e.g., U.S. Pat. No. 6,651,655, which is specifically incorporated by reference in its entirety). In one embodiment, the antigen-MHC-nanoparticle complex is administered systemically. In specific embodiments, the pMHC-NP complex or the compositions comprising a plurality of pMHC-NP complexes can be administered intravenously.

Typically, compositions of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner. However, suitable dosage ranges are of the order of ten to several hundred nanograms or micrograms of antigen/MHC/nanoparticle complex per administration. Suitable regimes for initial administration and boosters are also variable, but are typified by an initial administration followed by subsequent administrations.

The manner of application may be varied widely. Any of the conventional methods for administration of a vaccine are applicable. These are believed to include oral application on a solid physiologically acceptable base or in a physiologically acceptable dispersion, parenterally, by injection and the like. The dosage of the antigen/MHC/nanoparticle complex will depend on the route of administration and will vary according to the size and health of the subject.

In many instances, it will be desirable to have multiple administrations of a peptide/MHC/nanoparticle complex, about, at least about, or at most about 3, 4, 5, 6, 7, 8, 9, 10 or more administrations. The administrations will normally range from 1, 2, 3, 4, 5, 6, or 7 day to twelve week intervals, more usually from one to two week intervals. Periodic boosters at intervals of every other day, twice a week, weekly, biweekly, monthly, or 0.1, 0.2, 0.3, 0.4, 0.5, 1, 2, 3, 4 or 5 years, usually two years, will be desirable to maintain the condition of the immune system. The course of the administrations may be followed by assays for autoreactive immune responses, cognate T_{R}1 cells, and T cell activity.

In certain aspects, a single dose of the pMHC complex without including the nanoparticle core and any outer layer comprises about 0.001 mg/kg to about 2.0 mg/kg, or about 0.001 mg/kg to about 1.5 mg/kg, or about 0.001 mg/kg to about 1.4 mg/kg, or about 0.001 mg/kg to about 1.3 mg/kg, or about 0.001 mg/kg to about 1.2 mg/kg, or about 0.001 mg/kg to about 1.1 mg/kg, or about 0.001 mg/kg to about 1.0 mg/kg. In some embodiments, the single dose comprises from about 0.004 mg/kg to about 1.014 mg/kg, or from about 0.02 mg/kg to about 0.811 mg/kg, or from about 0.041 mg/kg to about 0.608 mg/kg, or from about 0.061 mg/kg to about 0.507 mg/kg, or from about 0.081 mg/kg to about 0.405 mg/kg, or from about 0.121 mg/kg to about 0.324 mg/kg, or from about 0.162 mg/kg to about 0.243 mg/kg. In some embodiments, the single dose comprises from about 0.004 mg/kg to about 1.015 mg/kg, or from about 0.004 mg/kg to about 1.0 mg/kg, or from about 0.004 mg/kg to about 0.9 mg/kg, or from about 0.004 mg/kg to about 0.8 mg/kg, or from about 0.004 mg/kg to about 0.7 mg/kg, or from about 0.004 mg/kg to about 0.6 mg/kg, or from about 0.004 mg/kg to about 0.5 mg/kg, or from about 0.004 mg/kg to about 0.4 mg/kg, or from about 0.004 mg/kg to about 0.3 mg/kg, or from about 0.004 mg/kg to about 0.2 mg/kg, or from about 0.004 mg/kg to about 0.1 mg/kg.

### Combination Therapy

The compositions and related methods of the present disclosure, particularly administration of an antigen/MHC/nanoparticle complex, may also be used in combination with the administration of traditional therapies. These include, but are not limited to, the administration of immunosuppressive or modulating therapies or treatments. Non-limiting examples of certain disease-relevant treatments include Avonex (interferon beta-1a), Betaseron (interferon beta-1b), Copaxone (glatiramer acetate), Novantrone (mitoxantrone), Rebif (interferon beta-1a), Tysabri (natalizumab), Gilenya (fingolimod), Glatiramer, steroids, Cytoxan, Imuran, Baclofen, deep brain stimulation, Ampyra (dalfampridine), acupuncture, and physical therapy. When treating cancer, additional chemotherapeutics, radiation or surgery may be added to augment the therapeutic response of the disclosed compositions and methods.

In one aspect, it is contemplated that an antigen/MHC/nanoparticle complex is used in conjunction with a cytokine treatment. Alternatively, antigen/MHC/nanoparticle complex administration may precede or follow the other treatment by intervals ranging from minutes to weeks. In embodiments where the other agents and/or antigen/MHC/nanoparticle complexes are administered separately, one would generally ensure that a significant period of time did not expire between the time of each delivery, such that the agent and antigen/MHC/nanoparticle complex would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

Various combinations may be employed, for example antigen/MHC/nanoparticle complex administration is “A” and the additional agent is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the peptide-MHC complex compositions of the present disclosure to a patient/subject will follow general protocols for the administration of such compounds, taking into account the toxicity, if any. It is expected that the treatment cycles would be repeated as necessary. It is also contemplated that various standard therapies, such as hydration, may be applied in combination with the described therapy.

### Pharmaceutical Carriers and Formulations

In some embodiments, pharmaceutical compositions are administered to a subject. Different aspects of the present disclosure involve administering an effective amount of a antigen/MHC/nanoparticle complex composition to a subject. Additionally, such compositions can be administered in combination with modifiers of the immune system. Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated.

The active compounds of the present disclosure can be formulated for parenteral administration, e.g., formulated for injection via the intravenous, intramuscular, sub-cutaneous, or even intraperitoneal routes. The preparation of an aqueous composition that contains a antigen/MHC/nanoparticle complex that modifies the subject's immune condition will be known to those of skill in the art in light of the present disclosure. Typically, such compositions can be prepared as injectables, either as liquid solutions or suspensions; solid forms suitable to prepare solutions or suspensions upon the addition of a liquid prior to injection; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases, the form must be sterile and must be fluid to the extent that it may be easily injected. It should also be stable under the conditions of manufacture and storage and must be preserved against the contaminating action of microorganisms, such as bacteria and fungi.

The compositions may be formulated into a neutral or salt form. Pharmaceutically acceptable salts, including the acid addition salts (formed with the free amino groups of the protein), are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by sterilization. Sterilization of the solution will be done in such a way as to not diminish the therapeutic properties of the antigen-MHC-nanoparticle complex. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the certain methods of preparation are vacuum-drying and freeze-drying techniques, which yield a powder of the active ingredient, plus any additional desired ingredient from a previously sterilized solution thereof. One such method of sterilization of the solution is sterile filtration, however, this disclosure is meant to include any method of sterilization that does not significantly decrease the therapeutic properties of the antigen-MHC-nanoparticle complexes. Methods of sterilization that involve intense heat and pressure, such as autoclaving, may compromise the tertiary structure of the complex, thus significantly decreasing the therapeutic properties of the antigen-MHC-nanoparticle complexes.

Administration of the compositions according to the present disclosure will typically be via any common route. This includes, but is not limited to, orthotopic, intradermal, subcutaneous, intramuscular, intraperitoneal, intranasal, or intravenous injection. In certain embodiments, a vaccine composition may be inhaled (e.g., U.S. Pat. No. 6,651,655, which is specifically incorporated by reference).

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above. A typical dosing regimen in a mouse model involves the administration of 1 μg-50 μg of total pMHC (NP-coated) and 1 μg-50 μg of total iron per dose which may be translated to a specific unit dosage in humans. In certain embodiments, the dose may range from about 0.1 μg to about 400 μg. However, it is understood that the amount of pMHC per dose can range from as low as 0.1 μg to 100 mg. As an example, in a 60 kg human patient, the amount of pMHC per dose can range from 0.24 mg to 12 mg with the understanding that this corresponds to the 1 μg to 50 μg discussed above. Also as above, this dose can be changed to correspond to 0.1 μg to 100 mg above, corresponding to a human equivalent dose of 0.0004 mg/kg to 405.4 mg/kg and ranges in between depending on the patient being treated, the condition and other parameters decided by the treating physician.

### In Vitro or Ex Vivo Administration

As used herein, the term in vitro administration refers to manipulations performed on cells removed from or outside of a subject, including, but not limited to cells in culture. The term ex vivo administration refers to cells which have been manipulated in vitro, and are subsequently administered to a subject. The term in vivo administration includes all manipulations performed within a subject, including administrations.

In certain aspects of the present disclosure, the compositions may be administered either in vitro, ex vivo, or in vivo. In certain in vitro embodiments, autologous T cells are incubated with compositions of this disclosure. The cells can then be used for in vitro analysis, or alternatively for ex vivo administration.

### Production of Protein Components

The present disclosure describes polypeptides, peptides, and proteins for use in various embodiments of the present disclosure. For example, specific peptides and their complexes are assayed for their abilities to elicit or modulate an immune response. In specific embodiments, all or part of the peptides or proteins of the disclosure can also be synthesized in solution or on a solid support in accordance with conventional techniques. Various automatic synthesizers are commercially available and can be used in accordance with known protocols.

Alternatively, recombinant DNA technology may be employed wherein a nucleotide sequence which encodes a peptide of the disclosure is inserted into an expression vector, transformed or transfected into an appropriate host cell and cultivated under conditions suitable for expression.

One embodiment of the disclosure includes the use of gene transfer to cells, including microorganisms, for the production of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are known to one skilled in the art and are briefly discussed herein. Examples of mammalian host cell lines include, but are not limited to, Vero and HeLa cells, other B- and T-cell lines, such as CEM, 721.221, H9, Jurkat, Raji, as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed.

A number of selection systems may be used including, but not limited to, HSV thymidine kinase, hypoxanthine-guanine phosphoribosyltransferase, and adenine phosphoribosyltransferase genes, in tk-, hgprt- or aprt-cells, respectively. Also, anti-metabolite resistance can be used as the basis of selection: for dhfr, which confers resistance to trimethoprim and methotrexate; gpt, which confers resistance to mycophenolic acid; neo, which confers resistance to the aminoglycoside G418; and hygro, which confers resistance to hygromycin.

### Nucleic Acids

The present disclosure may include recombinant polynucleotides encoding the proteins, polypeptides, peptides of the disclosure. The nucleic acid sequences for autoantigens and MHC molecules for presenting the autoantigens, are included and can be used to prepare a peptide/MHC complex.

As used in this disclosure, the term “polynucleotide” refers to a nucleic acid molecule that either is recombinant or has been isolated free of total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be RNA, DNA, analogs thereof, or a combination thereof.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide of the following lengths: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs. It is also contemplated that a particular polypeptide from a given species may be encoded by nucleic acids containing natural variations that having slightly different nucleic acid sequences but, nonetheless, encode the same or substantially similar protein, polypeptide, or peptide.

In particular embodiments, the disclosure concerns isolated nucleic acid segments and recombinant vectors incorporating nucleic acid sequences that encode an autoantigen and/or a MHC molecule. The term “recombinant” may be used in conjunction with a polypeptide or the name of a specific polypeptide, and this generally refers to a polypeptide produced from a nucleic acid molecule that has been manipulated in vitro or that is a replication product of such a molecule.

The nucleic acid segments used in the present disclosure, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. A tag or other heterologous polypeptide may be added to the modified polypeptide-encoding sequence, wherein “heterologous” refers to a polypeptide that is not the same as the modified polypeptide.

### Methods of Treatment

Medical and diagnostic methods are also provided. In one aspect, a method is provided for promoting the formation, expansion and recruitment of immune cells, including but not limited to, effector cells, B-regulatory cells and/or T_{R}1 cells (e.g., T_{R}1 and CD4+ cells) or CD8+ cells, in an antigen-specific manner in a subject in need thereof, comprising, or alternatively consisting essentially of, or yet further consisting of, administering to the subject an effective amount of the NP-complex or composition as described herein.

This disclosure also provides methods for differentiating or triggering T-regulatory type 1 (T_{R}1) cell formation in a pMHC dose independent manner. Applicant has discovered that the pMHC density on the nanoparticle core regulates the ability of pMHC on the nanoparticle core to trigger T_{R}1 cell formation in a dose-independent manner, while pMHC dose regulates the magnitude of T_{R}1 cell expansion in a pMHC density-independent manner. Applicant has observed that the pMHC density threshold and the independent effects of pMHC density versus dose on T_{R}1 cell formation versus expansion are unexpected findings that could not have been anticipated based on conventional immunological knowledge in the art. These methods require contacting the cognate T cells with an effective amount of a pMHC-NPor a composition disclosed herein. In certain aspects, the density-dependent methods relate to an activated T cell or a memory T cell being differentiated into a IL-10 producing cognate T_{R}1 cell optionally having the marker CD49b and/or Lag3 and/or a B cell being differentiated into a regulatory B cell by contacting the activated T cell or the memory T cell with an effective amount of the complex or composition disclosed herein. In some embodiments, the differentiated T regulatory cell binds to a B cell, thereby differentiating the B cell into a regulatory B cell. In certain aspects of the methods, the contacting is performed in vitro or in vivo.

Accordingly, aspects of the disclosure relate to a method for differentiating or triggering T_{R}1 cell formation in a pMHC dose independent manner comprising contacting the cognate T cells with an effective amount of the complex or composition disclosed herein. In certain aspects, the contacting may be in vitro or in vivo. In certain aspects, the methods relate to an activated T cell or a memory T cell being differentiated into a IL-10 producing T_{R}1 cell optionally expressing the marker CD49b and/or Lag3 comprising contacting the activated T cell or the memory T cell with an effective amount of the complex or composition disclosed herein. Based on the correlation between relevant cell type for each disease, the corresponding optimized MHC/NP complex and optionally co-stimulatory molecule and/or cytokine is also administered.

With this in mind, Applicant provides a method for differentiating an activated T cell or a memory T cell into a IL-10 producing T_{R}1 cell optionally expressing the marker CD49b and/or Lag3 and/or differentiating a B cell into a regulatory B cell comprising, or alternatively consisting of, or yet further consisting of, contacting the activated T cell or the memory T cell with an effective amount of the complex or composition as described herein. The contacting can be in vitro or in vivo. In some embodiments, the pMHC-NP or composition containing a plurality of the pMHC-NPs have pMHC-NPs having an average nanoparticle core diameter of from about 25 nm to about 60 nm, or from about 25 nm to about 50 nm, or from about 20 nm to about 40 nm, or from about 15 nm to about 50 nn, or from about 15 nm to about 40 nm, or from about 15 nm to about 35 nm, or from about 15 nm to about 30 nm, or from about 15 nm to about 25 nm, or alternatively about 15 nm, or about 20 nm, or about 25 nm, or about 30 nm, or about 35 nm, or about 40 nm. In some aspects, the nanoparticle core further comprises an outer coating or layer, wherein the diameter of the core and outer layer have an average diameter of from about 30 nm to about 75 nm, or from about 30 nm to about 70 nm, or from about 30 nm to about 60 nm, or from about 30 nm to about 50 nm, or about 40 nm. In some aspects, the nanoparticle has an average pMHC density of from about 0.4 pMHC/100 nm² to about 12 pMHC/100 nm², or from about 0.4 pMHC/100 nm² to about 11.6 pMHC/100 nm², or from about 0.4 pMHC/100 nm² to about 11.5 pMHC/100 nm², or from about 0.4 pMHC/100 nm² to about 11 pMHC/100 nm², or from about 0.4 pMHC/100 nm² to about 10 pMHC/100 nm², or from about 0.4 pMHC/100 nm² to about 9 pMHC/100 nm², or from about 0.4 pMHC/100 nm² to about 8 pMHC/100 nm², or from about 0.4 pMHC/100 nm² to about 7 pMHC/100 nm², or from about 0.4 pMHC/100 nm² to about 6 pMHC/100 nm², or from about 0.4 pMHC/100 nm² to about 5 pMHC/100 nm², or from about 0.4 pMHC/100 nm² to about 4 pMHC/100 nm², or from about 0.4 pMHC/100 nm² to about 3 pMHC/100 nm², or from about 0.4 pMHC/100 nm² to about 2.5 pMHC/100 nm², or from about 0.4 pMHC/100 nm² to about 2 pMHC/100 nm², or from about 0.4 pMHC/100 nm² to about 1.5 pMHC/100 nm².

Also provided are methods for differentiating an activated T cell or a memory T cell into a IL-10 producing T_{R}1 cell optionally expressing the marker CD49b and/or Lag3 and/or differentiating a B cell into a regulatory B cell comprising, or alternatively consisting essentially of, or yet further consisting of, administering to the subject an effective amount of the complex or composition as described herein. As used herein, the subject may include an animal, a mammal, a murine, a bovine, an equine, a canine, a feline, an ovine, or a human. In some embodiments, the pMHC-NP or composition containing a plurality of the pMHC-NPs have pMHC-NPs having an average nanoparticle core diameter of from about 25 nm to about 60 nm, or from about 25 nm to about 50 nm, or from about 20 nm to about 40 nm, or from about 15 nm to about 50 nn, or from about 15 nm to about 40 nm, or from about 15 nm to about 35 nm, or from about 15 nm to about 30 nm, or from about 15 nm to about 25 nm, or alternatively about 15 nm, or about 20 nm, or about 25 nm, or about 30 nm, or about 35 nm, or about 40 nm. In some aspects, the nanoparticle core further comprises an outer coating or layer, wherein the diameter of the core and outer layer have an average diameter of from about 30 nm to about 75 nm, or from about 30 nm to about 70 nm, or from about 30 nm to about 60 nm, or from about 30 nm to about 50 nm, or about 40 nm. In some aspects, the nanoparticle has an average pMHC density of from about 0.4 pMHC/100 nm² to about 12 pMHC/100 nm², or from about 0.4 pMHC/100 nm² to about 11.6 pMHC/100 nm², or from about 0.4 pMHC/100 nm² to about 11.5 pMHC/100 nm², or from about 0.4 pMHC/100 nm² to about 11 pMHC/100 nm², or from about 0.4 pMHC/100 nm² to about 10 pMHC/100 nm², or from about 0.4 pMHC/100 nm² to about 9 pMHC/100 nm², or from about 0.4 pMHC/100 nm² to about 8 pMHC/100 nm², or from about 0.4 pMHC/100 nm² to about 7 pMHC/100 nm², or from about 0.4 pMHC/100 nm² to about 6 pMHC/100 nm², or from about 0.4 pMHC/100 nm² to about 5 pMHC/100 nm², or from about 0.4 pMHC/100 nm² to about 4 pMHC/100 nm², or from about 0.4 pMHC/100 nm² to about 3 pMHC/100 nm², or from about 0.4 pMHC/100 nm² to about 2.5 pMHC/100 nm², or from about 0.4 pMHC/100 nm² to about 2 pMHC/100 nm², or from about 0.4 pMHC/100 nm² to about 1.5 pMHC/100 nm².

Provided herein are methods of treating an autoimmune disease or disorder in a subject in need thereof comprising administering an effective amount of any of the complexes or compositions disclosed herein to the subject, provided that the complexes and the compositions do not comprise co-stimulatory molecules.

Further provided herein are methods of treating a cancer or a tumor and/or inhibiting the growth of a tumor cell or tissue in a subject in need t hereof comprising administering an effective amount of any of the pMHC-NP complex with one or more co-stimulatory molecules.

Yet further aspects provided herein include a nanoparticle complex having a pMHC density of from about 0.4 pMHC/100 nm² to about 12 pMHC/100 nm² for use in promoting a differentiation of activated T cells or memory T cells into IL-10 producing T_{R}1 cells optionally expressing a marker CD49b and/or Lag3. In some embodiments, the pMHC-NP or composition containing a plurality of the pMHC-NPs have pMHC-NPs having an average nanoparticle core diameter of from about 25 nm to about 60 nm, or from about 25 nm to about 50 nm, or from about 20 nm to about 40 nm, or from about 15 nm to about 50 nn, or from about 15 nm to about 40 nm, or from about 15 nm to about 35 nm, or from about 15 nm to about 30 nm, or from about 15 nm to about 25 nm, or alternatively about 15 nm, or about 20 nm, or about 25 nm, or about 30 nm, or about 35 nm, or about 40 nm. In some aspects, the nanoparticle core further comprises an outer coating or layer, wherein the diameter of the core and outer layer have an average diameter of from about 30 nm to about 75 nm, or from about 30 nm to about 70 nm, or from about 30 nm to about 60 nm, or from about 30 nm to about 50 nm, or about 40 nm. In some aspects, the nanoparticle has an average pMHC density of from about 0.4 pMHC/100 nm² to about 12 pMHC/100 nm², or from about 0.4 pMHC/100 nm² to about 11.6 pMHC/100 nm², or from about 0.4 pMHC/100 nm² to about 11.5 pMHC/100 nm², or from about 0.4 pMHC/100 nm² to about 11 pMHC/100 nm², or from about 0.4 pMHC/100 nm² to about 10 pMHC/100 nm², or from about 0.4 pMHC/100 nm² to about 9 pMHC/100 nm², or from about 0.4 pMHC/100 nm² to about 8 pMHC/100 nm², or from about 0.4 pMHC/100 nm² to about 7 pMHC/100 nm², or from about 0.4 pMHC/100 nm² to about 6 pMHC/100 nm², or from about 0.4 pMHC/100 nm² to about 5 pMHC/100 nm², or from about 0.4 pMHC/100 nm² to about 4 pMHC/100 nm², or from about 0.4 pMHC/100 nm² to about 3 pMHC/100 nm², or from about 0.4 pMHC/100 nm² to about 2.5 pMHC/100 nm², or from about 0.4 pMHC/100 nm² to about 2 pMHC/100 nm², or from about 0.4 pMHC/100 nm² to about 1.5 pMHC/100 nm².

In one aspect, provided herein are methods for differentiating an activated T cell or a memory T cell into a IL-10 producing T_{R}1 cell expressing a marker comprising CD49b and/or LAG3, and/or differentiating a B cell into a regulatory B cell, the method comprising contacting the activated T cell or the memory T cell with an effective amount of a complex comprising: a nanoparticle core, wherein: a plurality of disease-relevant antigen-MHC (pMHC) complexes are coupled to the nanoparticle core; the diameter of the core is from about 15 nm to about 25 nm; and wherein the pMHC density on the nanoparticle is from about 0.4 pMHC/100 nm² to about 12 pMHC/100 nm² of the surface area of the nanoparticle. In some embodiments, the nanoparticle core further comprises an outer layer on the core, wherein the pMHC complex is coupled to the nanoparticle core and/or the outer layer, and wherein the combined diameter of the nanoparticle core and the outer layer is from about 35 nm to about 45 nm. In some embodiments, contacting is in vitro or in vivo.

In another aspect, provided herein is a nanoparticle complex for use in differentiating an activated T cell or a memory T cell into a IL-10 producing T_{R}1 cell expressing a marker comprising CD49b and/or LAG3, and/or differentiating a B cell into a regulatory B cell, wherein the nanoparticle complex comprises a nanoparticle core, wherein: a plurality of disease-relevant antigen-MHC (pMHC) complexes are coupled to the nanoparticle core; the diameter of the core is from about 15 nm to about 25 nm; and wherein the pMHC density on the nanoparticle is from about 0.4 pMHC/100 nm² to about 12 pMHC/100 nm² of the surface area of the nanoparticle.

In another aspect, provided herein is a nanoparticle complex for use in differentiating an activated T cell or a memory T cell into a IL-10 producing T_{R}1 cell expressing a marker comprising CD49b and/or LAG3, and/or differentiating a B cell into a regulatory B cell, wherein the nanoparticle complex comprises a nanoparticle core and an outer layer on the nanoparticle core, wherein: a plurality of disease-relevant antigen-MHC (pMHC) complexes are coupled to the nanoparticle core and/or the outer layer; the combined diameter of the core and the outer layer is from about 25 nm to about 45 nm; and wherein the pMHC density on the nanoparticle is from about 0.4 pMHC/100 nm² to about 12 pMHC/100 nm² of the surface area of the nanoparticle.

In some embodiments, a therapeutic effect comprises about a 0.1% to about a 250% increase in the population of T_{R}1 cells. In some embodiments, the increase comprises about 0.1% to about 225%, or about 0.1% to about 200%, or about 0.1% to about 175%, or about 0.1% to about 150%, or about 0.1% to about 125%, or about 0.1% to about 100%, or about 0.1% to about 75%, or about 0.1% to about 50%, or about 0.1% to about 25%, or about 0.1% to about 20%, or about 0.1% to about 15%, or about 0.1% to about 10%, or about 0.1% to about 9%, or about 0.1% to about 8%, or about 0.1% to about 7%, or about 0.1% to about 6%, or about 0.1% to about 5%, or about 0.1% to about 4%, or about 0.1% to about 3%, or about 0.1% to about 2%, or about 0.1% to about 1%, or about 0.1% to about 0.9%, or about 0.1% to about 0.8%, or about 0.1% to about 0.7%, or about 0.1% to about 0.6%, or about 0.1% to about 0.5%, or about 0.1% to about 0.4%, or about 0.1% to about 0.3%, or about 0.1% to about 0.2% increase in the population of T_{R}1 cells.

For the therapeutic use, the following diseases can be combined with the following antigen-MHC complexes and compositions containing them:

In some embodiments, the antigen of the pMHC complex comprises a:
a) a diabetes-relevant antigen and is derived from an antigen selected from one or more of the group: preproinsulin (PPI), islet-specific glucose-6-phosphatase (IGRP), glutamate decarboxylase (GAD), islet cell autoantigen-2 (ICA2), insulin, proinsulin, or a fragment or an equivalent of each thereof;
b) a multiple sclerosis-relevant antigen and is derived from an antigen selected from one or more of the group: myelin basic protein, myelin associated glycoprotein, myelin oligodendrocyte protein, proteolipid protein, oligodendrocyte myelin oligoprotein, myelin associated oligodendrocyte basic protein, oligodendrocyte specific protein, heat shock proteins, oligodendrocyte specific proteins, NOGO A, glycoprotein Po, peripheral myelin protein 22, 2′3′-cyclic nucleotide 3′-phosphodiesterase, or a fragment or an equivalent of each thereof;
c) a Celiac Disease-relevant antigen and is derived from gliadin or a fragment or an equivalent thereof;
d) a primary biliary cirrhosis-relevant antigen and is derived from PDC-E2 or a fragment or an equivalent thereof;
e) a pemphigus folliaceus-relevant antigen and/or pemphigus vulgaris-relevant antigen and is derived from an antigen selected from one or more of the group: DG1, DG3, or a fragment or an equivalent of each thereof;
f) a neuromyelitis optica spectrum disorder-relevant antigen and is derived from AQP4 or a fragment or an equivalent thereof;
g) an arthritis-relevant antigen and is derived from an antigen selected from one or more of the group: heat shock proteins, immunoglobulin binding protein, heterogeneous nuclear RNPs, annexin V, calpastatin, type II collagen, glucose-6-phosphate isomerase, elongation factor human cartilage gp39, mannose binding lectin, citrullinated vimentin, type II collagen, fibrinogen, alpha enolase, anti-carbamylated protein (anti-CarP), peptidyl arginine deiminase type 4 (PAD4), BRAF, fibrinogen gamma chain, inter-alpha-trypsin inhibitor heavy chain H1, alpha-1-antitrypsin, plasma protease C1 inhibitor, gelsolin, alpha 1-B glycoprotein, ceruloplasmin, inter-alpha-trypsin inhibitor heavy chain H4, complement factor H, alpha 2 macroglobulin, serum amyloid, C-reactive protein, serum albumin, fibrogen beta chain, serotransferin, alpha 2 HS glycoprotein, vimentin, Complement C3, or a fragment or an equivalent of each thereof;
h) an allergic asthma-relevant antigen and is derived from an antigen selected from one or more of the group: DERP1, DERP2, or a fragment or an equivalent of each thereof;
i) an inflammatory bowel disease-relevant antigen and is derived from an antigen selected from one or more of the group: Flagelin, Fla-2, Fla-X, YIDX, bacteroides integrase, or a fragment or an equivalent of each thereof;
j) a systemic lupus erythematosus-relevant antigen and is derived from an antigen selected from one or more of the group: double-stranded (ds)DNA, ribonucleoprotein (RNP), Smith (Sm), Sjögren's-syndrome-related antigen A (SS-A)/Ro, Sjögren's-syndrome-related antigen B (SS-B)/La, RO60, RO52, histones, or a fragment or an equivalent of each thereof;
k) an atherosclerosis-relevant antigen and is derived from an antigen selected from one or more of the group: ApoB, ApoE or a fragment or an equivalent of each thereof;
l) a COPD-relvant antigen and/or emphysema-relevant antigen and is derived from elastin or a fragment or an equivalent thereof;
m) a psoriasis-relevant antigen and is derived from an antigen selected from one or more of the group: Cap18, ADMTSL5, ATL5, or a fragment or an equivalent of each thereof;
n) an autoimmune hepatitis-relevant antigen and is derived from an antigen selected from one or more of the group: CYP2D6, SLA, or a fragment or an equivalent of each thereof;
o) an uveitis-relevant antigen and is derived from arrestin or a fragment or an equivalent thereof;
p) a Sjogren's Syndrome-relevant antigen and is derived from an antigen selected from one or more of the group: (SS-A)/Ro, (SS-B)/La, MR3, RO60, RO52, or a fragment or an equivalent of each thereof;
q) a scleroderma-relevant antigen and is derived from an antigen selected from one or more of the group: CENP-C, TOP 1, RNA polymerase III, or a fragment or an equivalent of each thereof;
r) an anti-phospholipid syndrome-relevant antigen and is derived from APOH or a fragment or an equivalent thereof;
s) an ANCA-associated vasculitis-relevant antigen and is derived from an antigen selected from one or more of the group: MPO, PRTN3, or a fragment or an equivalent of each thereof; or
t) a Stiff Man Syndrome-relevant antigen and is derived from GAD or a fragment or an equivalent thereof.

In some embodiments, the MHC protein of the pMHC complex comprises all or part of a classical MHC class I protein, non-classical MHC class I protein, classical MHC class II protein, non-classical MHC class II protein, MHC dimers (Fc fusions), MHC tetramers, or a polymeric form of a MHC protein, wherein the MHC protein optionally comprises a knob-in-hole based MHC-alpha-Fc/MHC-beta-Fc heterodimer or multimer.

In some embodiments, the MHC protein of the pMHC complex comprises all or part of a polypeptide of the group: HLA DR, HLA DQ, HLA DP, HLA-A, HLA-B, HLA-C, HLA-E, HLA-F, HLA-G, CD1d, or a fragment or an equivalent of each thereof.

In some embodiments, the MHC protein of the pMHC complex comprises all or part of a polypeptide of the group: HLA-DR, HLA-DQ, HLA-DP, or a fragment or an equivalent of each thereof.

In some embodiments, the MHC protein of the pMHC complex comprises all or part of a polypeptide of the group: HLA-DRB1/DRA, HLA-DRB3/DRA, HLA-DRB4/DRA, HLA-DRB5/DRA, HLA-DQA1/HLA-DQB1, HLA-DPB1/HLA-DPA1, or a fragment or an equivalent of each thereof.

In certain aspects, the pMHC complex comprises:
a) a diabetes-relevant antigen derived from an antigen selected from one or more of the group: hInsB₁₀₋₁₈, hIGRP₂₂₈₋₂₃₆, hIGRP₂₆₅₋₂₇₃, IGRP₂₀₆₋₂₁₄, hIGRP₂₀₆₋₂₁₄, NRP-A7, NRP-I4, NRP-V7, YAI/D^{b}, INS B₁₅₋₂₃, PPI_{76-90 (K88S)}, IGRP₁₃₋₂₅, GAD₅₅₅₋₅₆₇, GAD_{555-567(557I)}, IGRP₂₃₋₃₅, B₂₄-C₃₆, PPI₇₆₋₉₀, INS-I9, TUM, G6Pase, Pro-insulin_{L2-10}, Pro-insulin_{L3-11}, Pro-insulin_{L6-14}, Pro-insulin_{B5-14}, Pro-insulin_{B10-18}, Pro-insulin_{B14-22}, Pro-insulin_{B15-24}, Pro-insulin_{B17-25}, Pro-insulin_{B18-27}, Pro-insulin_{B20-27}, Pro-insulin_{B21-29}, Pro-insulin_{B25-C1}, Pro-insulin_{B27-C5}, Pro-insulin_{C20-28}, Pro-insulin_{C25-33}, Pro-insulin_{C29-A5}, Pro-insulin_{A1-10}, Pro-insulin_{A2-10}, Pro-insulin_{A12-20}, or a fragment or an equivalent of each thereof;
b) a multiple sclerosis-relevant antigen derived from an antigen selected from one or more of the group: MOG₃₅₋₅₅, MOG₃₆₋₅₅, MAG₂₈₇₋₂₉₅, MAG₅₀₉₋₅₁₇, MAG₅₅₆₋₅₆₄, MBP₁₁₀₋₁₁₈, MOG₁₁₄₋₁₂₂, MOG₁₆₆₋₁₇₅, MOG₁₇₂₋₁₈₀, MOG₁₇₉₋₁₈₈, MOG₁₈₈₋₁₉₆, MOG₁₈₁₋₁₈₉, MOG₂₀₅₋₂₁₄, PLP₈₀₋₈₈, MAG₂₈₇₋₂₉₅, MAG₅₀₉₋₅₁₇, MAG₅₅₆₋₅₆₄, MOG₉₇₋₁₀₉ MOG_{97-109(E107S)}, MBP₈₉₋₁₀₁, PLP₁₇₅₋₁₉₂, PLP₉₄₋₁₀₈, MBP₈₆₋₉₈, PLP₅₄₋₆₈, PLP₂₄₉₋₂₆₃, MOG₁₅₆₋₁₇₀, MOG₂₀₁₋₂₁₅, MOG₃₈₋₅₂, MOG₂₀₃₋₂₁₇, PLP₂₅₀₋₂₆₄, MPB₁₃₋₃₂, MPB₈₃₋₉₉, MPB₁₁₁₋₁₂₉, MPB₁₄₆₋₁₇₀, MOG₂₂₃₋₂₃₇, MOG₆₋₂₀, PLP₈₈₋₁₀₂, PLP₁₃₉₋₁₅₄, or a fragment or an equivalent of each thereof;
c) a Celiac Disease-relevant antigen derived from an antigen selected from one or more of the group: aGlia₅₇₋₆₈, aGlia₆₂₋₇₂, aGlia₂₁₇₋₂₂₉, or a fragment or an equivalent of each thereof;
d) a primary biliary cirrhosis-relevant antigen derived from an antigen selected from one or more of the group: PDC-E2₁₂₂₋₁₃₅, PDC-E2₂₄₉₋₂₆₂, PDC-E2₂₄₉₋₂₆₃, PDC-E2₆₂₉₋₆₄₃, PDC-E₂₇₂₋₈₆, PDC-E2₃₅₃₋₃₆₇, PDC-E2₄₂₂₋₄₃₆, PDC-E2₆₂₉₋₆₄₃, PDC-E2₈₀₋₉₄, PDC-E2₃₅₃₋₃₆₇, PDC-E₂₅₃₅₋₅₄₉, or a fragment or an equivalent of each thereof;
e) a pemphigus folliaceus-relevant antigen and/or pemphigus vulgaris-relevant antigen, each of which is derived from an antigen selected from one or more of the group: DG1₂₁₆₋₂₂₉, DG3₉₇₋₁₁₁, DG3₂₅₁₋₂₆₅, DG3₄₄₁₋₄₅₅, DG3₃₅₁₋₃₆₅, DG3₄₅₃₋₄₆₇, DG3₅₄₀₋₅₅₄, DG3₂₈₀₋₂₉₄, DG3₃₂₆₋₃₄₀, DG3₃₆₇₋₃₈₁, DG3₁₃₋₂₇, DG3₃₂₃₋₃₃₇, DG3₄₃₈₋₄₅₂, DG1₄₈₋₆₂, DG1₂₀₆₋₂₂₂, DG1₃₆₃₋₃₇₇, DG1₃₋₁₇, DG1₁₉₂₋₂₀₆, DG1₃₂₆₋₃₄₀, DG1₁₋₁₅, DG1₃₅₋₄₉, DG1₃₂₅₋₃₃₉, or a fragment or an equivalent of each thereof;
f) a neuromyelitis optica spectrum disorder-relevant antigen derived from an antigen selected from one or more of the group: AQP4₁₂₉₋₁₄₃, AQP4₂₈₄₋₂₉₈, AQP4₆₃₋₇₆, AQP4₁₂₉₋₁₄₃, AQP4₃₉₋₅₃, or a fragment or an equivalent of each thereof;
g) an allergic asthma-relevant antigen derived from an antigen selected from one or more of the group: DERP1₁₆₋₃₀, DERP1₁₇₁₋₁₈₅, DERP1₁₁₀₋₁₂₄, DERP-2₂₆₋₄₀, DERP-2₁₀₇₋₁₂₁, or a fragment or an equivalent of each thereof;
h) an inflammatory bowel disease-relevant antigen derived from an antigen selected from one or more of the group: bacteroides integrase antigen₁₈₃₋₁₉₇, bacteroides integrase antigen₁₄₆₋₁₆₀, bacteroides integrase antigen₁₇₅₋₁₈₉, bacteroides integrase antigen₁₋₁₅, bacteroides integrase antigen₁₈₃₋₁₉₇, bacteroides integrase antigen₃₀₋₄₄, bacteroides integrase antigen₇₀₋₈₄, bacteroides integrase antigen₃₃₇₋₃₅₁, bacteroides integrase antigen₁₇₁₋₁₈₅, bacteroides integrase antigen₄₋₁₈, bacteroides integrase antigen₂₅₆₋₂₇₀, Fla-2/Fla-X₃₆₆₋₃₈₀, Fla-2/Fla-X₁₆₄₋₁₇₈, Fla-2/Fla-X₂₆₁₋₂₇₅, Fla-2/Fla-X₁₋₁₅, Fla-2/Fla-X₅₁₋₆₅, Fla-2/Fla-X₂₆₉₋₂₈₃, Fla-2/Fla-X₄₋₁₈, Fla-2/Fla-X₂₇₁₋₂₈₅, YIDX₇₈₋₉₂, YIDX₉₃₋₁₀₇, YIDX₉₈₋₁₁₂, YIDX₂₃₋₃₇, YIDX₇₈₋₉₂, YIDX₁₉₅₋₂₀₉, YIDX₂₂₋₃₆, YIDX₈₀₋₉₄, YIDX₁₀₁₋₁₁₅, or a fragment or an equivalent of each thereof;
i) a systemic lupus erythematosus-relevant antigen derived from an antigen selected from one or more of the group: H4₇₁₋₉₄, H4₇₄₋₈₈, H4₇₆₋₉₀, H4₇₅₋₈₉, H4₇₈₋₉₂, H4₈₀₋₉₄, H2B₁₀₋₂₄, H2B₁₆₋₃₀, H1′₂₂₋₄₂, H1′₂₇₋₄₁, or a fragment or an equivalent of each thereof;
j) an atherosclerosis-relevant antigen derived from an antigen selected from one or more of the group: ApoB₃₅₀₁₋₃₅₁₆, ApoB₁₉₅₂₋₁₉₆₆, ApoB₉₇₈₋₉₉₃, ApoB₃₄₉₈₋₃₅₁₃, ApoB_{210A}, ApoB_{210B}, ApoB_{210C}, or a fragment or an equivalent of each thereof;
k) a COPD-relvant antigen and/or emphysema-relevant antigen, each of which is derived from an antigen selected from one or more of the group: elastin₈₉₋₁₀₃, elastin₆₉₈₋₇₁₂, elastin₈₋₂₂, elastin₉₄₋₁₀₈, elastin₁₃₋₂₇, elastin₆₉₅₋₇₀₉, elastin₅₆₃₋₅₇₇, elastin₅₅₈₋₅₇₂, elastin₆₉₈₋₇₁₂, elastin₅₆₆₋₅₈₀, elastin₅₄₅₋₆₅₉, or a fragment or an equivalent of each thereof;
l) a psoriasis-relevant antigen derived from an antigen selected from one or more of the group: Cap18₆₄₋₇₈, Cap18₃₄₋₄₈, Cap18₄₇₋₆₁, Cap18₁₅₁₋₁₆₅, Cap18₁₄₉₋₁₆₃, Cap18₁₅₂₋₁₆₆, Cap18₁₃₁₋₁₄₅, Cap18₂₄₋₃₈, ADMTSL5245₋₂₅₉, ADMTSL5₂₆₇₋₂₈₁, ADMTSL5₃₇₂₋₃₈₆, ADMTSL5₂₈₉₋₃₀₃, ADMTSL5₃₉₆₋₄₁₀, ADMTSL5₄₃₃₋₄₄₇, ADMTSL5₁₄₂₋₁₅₆, ADMTSL5₂₃₆₋₂₅₀, ADMTSL5₃₀₁₋₃₁₅, ADMTSL5₂₀₃₋₂₁₇, ADMTSL5₄₀₄₋₄₁₈, or a fragment or an equivalent of each thereof;
m) an autoimmune hepatitis-relevant antigen derived from an antigen selected from one or more of the group: (CYP2D6)₁₉₃₋₂₀₇, CYP2D6₇₆₋₉₀, CYP2D6₂₉₃₋₃₀₇, CYP2D6₃₁₃₋₃₃₂, CYP2D6₃₉₃₋₄₁₂, CYP2D6₁₉₉₋₂₁₃, CYP2D6₄₅₀₋₄₆₄, CYP2D6₃₀₁₋₃₁₅, CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₃₁₋₁₄₅, CYP2D6₂₁₆₋₂₃₀, CYP2D6₂₃₈₋₂₅₂, CYP2D6₁₉₉₋₂₁₃, CYP2D6₂₃₅₋₂₅₂, CYP2D6₂₉₃₋₃₀₇, CYP2D6₃₈₁₋₃₉₅, CYP2D6₄₂₉₋₄₄₃, SLA₃₃₄₋₃₄₈, SLA₁₉₆₋₂₁₀, SLA₁₁₅₋₁₂₉, SLA₃₇₃₋₃₈₆, SLA₁₈₆₋₁₉₇, SLA₃₁₇₋₃₃₁, SLA₁₇₁₋₁₈₅, SLA₄₁₇₋₄₃₁, SLA₃₅₉₋₃₇₃, SLA₂₁₅₋₂₂₉, SLA₁₁₁₋₁₂₅, SLA₁₁₀₋₁₂₄, SLA₂₉₉₋₃₁₃, SLA₃₄₂₋₃₅₆, SLA₄₉₋₆₃, SLA₁₁₉₋₁₃₃, SLA₂₆₀₋₂₇₄, SLA₂₆₋₄₀, SLA₈₆₋₁₀₀, SLA₃₃₁₋₃₄₅, or a fragment or an equivalent of each thereof;
n) an uveitis-relevant antigen derived from an antigen selected from one or more of the group: arrestin₁₉₉₋₂₁₃, arrestin₇₇₋₉₁, arrestin₂₅₀₋₂₆₄, arrestin₁₇₂₋₁₈₆, arrestin₃₅₄₋₃₆₈, arrestin₂₃₉₋₂₅₃, arrestin₁₀₂₋₁₁₆, arrestin₅₉₋₇₃, arrestin₂₈₀₋₂₉₄, arrestin₂₉₁₋₃₀₆, arrestin₁₉₅₋₂₀₉, arrestin₂₀₀₋₂₁₄, or a fragment or an equivalent of each thereof;
o) a Sjogren's Syndrome-relevant antigen derived from an antigen selected from one or more of the group: RO60₁₂₇₋₁₄₁, RO60₅₂₃₋₅₃₇, RO60₂₄₃₋₂₅₇, RO60₄₈₄₋₄₉₈, RO60₃₄₇₋₃₆₁, RO60₃₆₉₋₃₈₃, RO60₄₂₆₋₄₄₀, RO60₂₆₇₋₂₈₁, RO60₁₇₈₋₁₉₂, RO60₃₅₈₋₃₇₂, RO60₂₂₁₋₂₃₅, RO60₃₁₈₋₃₃₂, RO60₄₀₇₋₄₂₁, RO60₄₅₉₋₄₇₃, RO60₅₁₋₆₅, RO60₃₁₂₋₃₂₆, LA₂₄₁₋₂₅₅, LA₁₀₁₋₁₁₅, LA₁₅₃₋₁₆₇, LA₁₇₈₋₁₉₂, LA₁₉₋₃₃, LA₃₇₋₅₁, LA₁₃₃₋₁₄₇, LA₅₀₋₆₄, LA₃₂₋₄₆, LA₁₅₃₋₁₆₇, LA₈₃₋₉₇, LA₁₃₆₋₁₅₀, LA₂₉₇₋₃₁₁, LA₅₉₋₇₃, LA₁₅₁₋₁₆₅, LA₈₆₋₁₀₀, LA₁₅₄₋₁₆₈, or a fragment or an equivalent of each thereof;
p) a scleroderma-relevant antigen derived from an antigen selected from one or more of the group: TOP1₃₄₆₋₃₆₀, TOP1₄₂₀₋₄₃₄, TOP1₇₅₀₋₇₆₄, TOP1₄₁₉₋₄₃₃, TOP1₅₉₁₋₆₀₅, TOP1₆₉₅₋₇₀₉, TOP1₃₀₅₋₃₁₉, TOP1₃₄₆₋₃₆₀, TOP1₄₁₉₋₄₃₃, TOP1₄₂₅₋₄₃₉, TOP1₆₁₄₋₆₂₈, CENP-C₂₉₇₋₃₁₁, CENP-C₈₅₇₋₈₇₁, CENP-C₈₈₇₋₉₀₁, CENP-C₂₁₂₋₂₂₆, CENP-C₆₄₃₋₆₅₇, CENP-C₈₃₂₋₈₄₆, CENP-C₁₆₇₋₁₈₁, CENP-C₂₄₆₋₂₆₀, CENP-C₈₄₆₋₈₆₀, CENP-C₁₄₉₋₁₆₃, CENP-C₈₃₃₋₈₄₇, CENP-C₈₄₇₋₈₆₁, or a fragment or an equivalent of each thereof;
q) an anti-phospholipid syndrome-relevant antigen derived from an antigen selected from one or more of the group: APOH₂₃₅₋₂₄₉, APOH₃₀₆₋₃₂₀, APOH₂₃₇₋₂₅₁, APOH₂₉₅₋₃₀₉, APOH₂₈₄₂, APOH₁₇₃₋₁₈₇, APOH₂₆₄₋₂₇₈, APOH₂₉₅₋₃₀₉, APOH₄₉₋₆₃, APOH₂₆₉₋₂₈₃, APOH₂₉₅₋₃₀₉, APOH₃₂₁₋₃₅₅, APOH₃₂₂₋₃₃₆, APOH₃₂₄₋₃₃₈, or a fragment or an equivalent of each thereof;
r) an ANCA-associated vasculitis-relevant antigen derived from an antigen selected from one or more of the group: MPO₅₀₆₋₅₂₀, MPO₃₀₂₋₃₁₆, MPO₇₋₂₁, MPO₆₈₉₋₇₀₃, MPO₂₄₈₋₂₆₂, MPO₄₄₄₋₄₅₈, MPO₅₁₃₋₅₂₇, MPO₉₇₋₁₁₁, MPO₆₁₆₋₆₃₀, MPO₄₆₂₋₄₇₆, MPO₆₁₇₋₆₃₁, MPO₇₁₄₋₇₂₈, PRTN3₄₄₋₅₈, PRTN3₂₃₄₋₂₄₈, PRTN3₅₉₋₇₃, PRTN3₁₁₇₋₁₃₁, PRTN3₁₆₄₋₁₇₈, PRTN3₇₁₋₈₅, PRTN3₂₄₁₋₂₅₅, PRTN3₅₉₋₇₃, PRTN3₁₈₃₋₁₉₇, PRTN3₆₂₋₇₆, PRTN3₁₁₈₋₁₃₂, PRTN3₂₃₉₋₂₅₃, or a fragment or an equivalent of each thereof; or
s) a Stiff Man Syndrome-relevant antigen derived from an antigen selected from one or more of the group: GAD₂₁₂₋₂₂₆, GAD₅₅₅₋₅₆₉, GAD₂₉₇₋₃₁₁, or a fragment or an equivalent of each thereof.

In certain aspects, the pMHC complex comprises:
a) a diabetes-relevant antigen derived from an antigen selected from one or more of the group: hInsB₁₀₋₁₈, hIGRP₂₂₈₋₂₃₆, hIGRP₂₆₅₋₂₇₃, IGRP₂₀₆₋₂₁₄, hIGRP₂₀₆₋₂₁₄, NRP-A7, NRP-I4, NRP-V7, YAI/D^{b}, INS B₁₅₋₂₃, PPI_{76-90 (K88S)}, IGRP₁₃₋₂₅, GAD₅₅₅₋₅₆₇, GAD_{555-567(557I)}, IGRP₂₃₋₃₅, B₂₄-C₃₆, PPI₇₆₋₉₀, INS-I9, TUM, G6Pase, Pro-insulin_{L2-10}, Pro-insulin_{L3-11}, Pro-insulin_{L6-14}, Pro-insulin_{B5-14}, Pro-insulin_{B10-18}, Pro-insulin_{B14-22}, Pro-insulin_{B15-24}, Pro-insulin_{B17-25}, Pro-insulin_{B18-27}, Pro-insulin_{B20-27}, Pro-insulin_{B21-29}, Pro-insulin_{B25-C1}, Pro-insulin_{B27-C5}, Pro-insulin_{C20-28}, Pro-insulin_{C25-33}, Pro-insulin_{C29-A5}, Pro-insulin_{A1-10}, Pro-insulin_{A2-10}, Pro-insulin_{A12-20}, or a fragment or an equivalent of each thereof, and the MHC protein of the pMHC complex comprises all or part of HLA-DR or a fragment or an equivalent thereof;
b) a multiple sclerosis-relevant antigen derived from an antigen selected from one or more of the group: MOG₃₅₋₅₅, MOG₃₆₋₅₅, MAG₂₈₇₋₂₉₅, MAG₅₀₉₋₅₁₇, MAG₅₅₆₋₅₆₄, MBP₁₁₀₋₁₁₈, MOG₁₁₄₋₁₂₂, MOG₁₆₆₋₁₇₅, MOG₁₇₂₋₁₈₀, MOG₁₇₉₋₁₈₈, MOG₁₈₈₁₉₆, MOG₁₈₈₋₁₈₉, MOG₂₀₅₋₂₁₄, PLP₈₀₋₈₈, MAG₂₈₇₋₂₉₅, MAG₅₀₉₋₅₁₇, MAG₅₅₆₋₅₆₄, MOG₉₇₋₁₀₉ MOG_{97-109(E107S)}, MBP₈₉₁₀₁, PLP₁₇₅₋₁₉₂, PLP₉₄₋₁₀₈, MBP₈₆₋₉₈, PLP₅₄₋₆₈, PLP₂₄₉₋₂₆₃, MOG₁₅₆₋₁₇₀, MOG₂₀₁₋₂₁₅, MOG₃₈₋₅₂, MOG₂₀₃₋₂₁₇, PLP₂₅₀₋₂₆₄, MPB₁₃₋₃₂, MPB₈₃₋₉₉, MPB₁₁₁₋₁₂₉, MPB₁₄₆₋₁₇₀, MOG₂₂₃₋₂₃₇, MOG₆₋₂₀, PLP₈₈₋₁₀₂, PLP₁₃₉₋₁₅₄, or a fragment or an equivalent of each thereof, and the MHC protein of the pMHC complex comprises all or part of HLA-DR or a fragment or an equivalent thereof;
c) a Celiac Disease-relevant antigen derived from an antigen selected from one or more of the group: aGlia₅₇₋₆₈, aGlia₆₂₋₇₂, aGlia₂₁₇₋₂₂₉, or a fragment or an equivalent of each thereof, and the MHC protein of the pMHC complex comprises all or part of HLA-DQ or a fragment or an equivalent thereof;
d) a primary biliary cirrhosis-relevant antigen derived from an antigen selected from one or more of the group: PDC-E2₁₂₂₋₁₃₅, PDC-E2₂₄₉₋₂₆₂, PDC-E2₂₄₉₋₂₆₃, PDC-E2₆₂₉₋₆₄₃, PDC-E₂₇₂₋₈₆, PDC-E2₃₅₃₋₃₆₇, PDC-E2₄₂₂₋₄₃₆, PDC-E2₆₂₉₋₆₄₃, PDC-E2₈₀₋₉₄, PDC-E2₃₅₃₋₃₆₇, PDC-E₂₅₃₅₋₅₄₉, or a fragment or an equivalent of each thereof, and the MHC protein of the pMHC complex comprises all or part of HLA-DR or a fragment of an equivalent thereof;
e) a pemphigus folliaceus-relevant antigen and/or pemphigus vulgaris-relevant antigen, each of which is derived from an antigen selected from one or more of the group: DG1₂₁₆₋₂₂₉, DG3₉₇₋₁₁₁, DG3₂₅₁₋₂₆₅, DG3₄₄₁₋₄₅₅, DG3₃₅₁₋₃₆₅, DG3₄₅₃₋₄₆₇, DG3₅₄₀₋₅₅₄, DG3₂₈₀₋₂₉₄, DG3₃₂₆₋₃₄₀, DG3₃₆₇₋₃₈₁, DG3₁₃₋₂₇, DG3₃₂₃₋₃₃₇, DG3₄₃₈₋₄₅₂, DG1₄₈₋₆₂, DG1₂₀₆₋₂₂₂, DG1₃₆₃₋₃₇₇, DG1₃₋₁₇, DG1₁₉₂₋₂₀₆, DG1₃₂₆₋₃₄₀, DG1₁₋₁₅, DG1₃₅₋₄₉, DG1₃₂₅₋₃₃₉, or a fragment or an equivalent of each thereof, and the MHC protein of the pMHC complex comprises all or part of HLA-DR or a fragment or an equivalent thereof;
f) a neuromyelitis optica spectrum disorder-relevant antigen derived from an antigen selected from one or more of the group: AQP4₁₂₉₋₁₄₃, AQP4₂₈₄₋₂₉₈, AQP4₆₃₋₇₆, AQP4₁₂₉₋₁₄₃, AQP4₃₉₋₅₃, or a fragment or an equivalent of each thereof, and the MHC protein of the pMHC complex comprises all or part of HLA-DR or a fragment or an equivalent thereof;
g) an allergic asthma-relevant antigen derived from an antigen selected from one or more of the group: DERP1₁₆₋₃₀, DERP1₁₇₁₋₁₈₅, DERP1₁₁₀₋₁₂₄, DERP-2₂₆₋₄₀, DERP-2₁₀₇₋₁₂₁, or a fragment or an equivalent of each thereof, and the MHC protein of the pMHC complex comprises all or part of a polypeptide of the group: HLA-DR, HLA-DP, or a fragment or an equivalent of each thereof;
h) an inflammatory bowel disease-relevant antigen derived from an antigen selected from one or more of the group: bacteroides integrase antigen₁₈₃₋₁₉₇, bacteroides integrase antigen₁₄₆₋₁₆₀, bacteroides integrase antigen₁₇₅₋₁₈₉, bacteroides integrase antigen₁₋₁₅, bacteroides integrase antigen₁₈₃₋₁₉₇, bacteroides integrase antigen₃₀₋₄₄, bacteroides integrase antigen₇₀₋₈₄, bacteroides integrase antigen₃₃₇₋₃₅₁, bacteroides integrase antigen₁₇₁₋₁₈₅, bacteroides integrase antigen₄₋₁₈, bacteroides integrase antigen₂₅₆₋₂₇₀, Fla-2/Fla-X₃₆₆₋₃₈₀, Fla-2/Fla-X₁₆₄₋₁₇₈, Fla-2/Fla-X₂₆₁₋₂₇₅, Fla-2/Fla-X₁₋₁₅, Fla-2/Fla-X₅₁₋₆₅, Fla-2/Fla-X₂₆₉₋₂₈₃, Fla-2/Fla-X₄₋₁₈, Fla-2/Fla-X₂₇₁₋₂₈₅, YIDX₇₈₋₉₂, YIDX₉₃₋₁₀₇, YIDX₉₈₋₁₁₂, YIDX₂₃₋₃₇, YIDX₇₈₋₉₂, YIDX₁₉₅₋₂₀₉, YIDX₂₂₋₃₆, YIDX₈₀₋₉₄, YIDX₁₀₁₋₁₁₅, or a fragment or an equivalent of each thereof, and the MHC protein of the pMHC complex comprises all or part of HLA-DR or a fragment or an equivalent thereof;
i) a systemic lupus erythematosus-relevant antigen derived from an antigen selected from one or more of the group: H4₇₁₋₉₄, H4₇₄₋₈₈, H4₇₆₋₉₀, H4₇₅₋₈₉, H4₇₈₋₉₂, H4₈₀₋₉₄, H2B₁₀₋₂₄, H2B₁₆₋₃₀, H1′₂₂₋₄₂, H1′₂₇₋₄₁, or a fragment or an equivalent of each thereof, and the MHC protein of the pMHC complex comprises all or part of a polypeptide of the group: I-A_{d}, HLA-DR, or a fragment or an equivalent of each thereof;
j) an atherosclerosis-relevant antigen derived from an antigen selected from one or more of the group: ApoB₃₅₀₁₋₃₅₁₆, ApoB₁₉₅₂₋₁₉₆₆, ApoB₉₇₈₋₉₉₃, ApoB₃₄₉₈₋₃₅₁₃, ApoB_{210A}, ApoB_{210B}, ApoB_{210C}, or a fragment or an equivalent of each thereof, and the MHC protein of the pMHC complex comprises all or part of I-A_{b} or a fragment or an equivalent thereof;
k) a COPD-relvant antigen and/or emphysema-relevant antigen, each of which is derived from an antigen selected from one or more of the group: elastin₈₉₋₁₀₃, elastin₆₉₈₋₇₁₂, elastin₈₋₂₂, elastin₉₄₋₁₀₈, elastin₁₃₋₂₇, elastin₆₉₅₋₇₀₉, elastin₅₆₃₋₅₇₇, elastin₅₅₈₋₅₇₂, elastin₆₉₈₋₇₁₂, elastin₅₆₆₋₅₈₀, elastin₅₄₅₋₆₅₉, or a fragment or an equivalent of each thereof, and the MHC protein of the pMHC complex comprises all or part of HLA-DR or a fragment or an equivalent thereof;
l) a psoriasis-relevant antigen derived from an antigen selected from one or more of the group: Cap18₆₄₋₇₈, Cap18₃₄₋₄₈, Cap18₄₇₋₆₁, Cap18₁₅₁₋₁₆₅, Cap18₁₄₉₋₁₆₃, Cap18₁₅₂₋₁₆₆, Cap18₁₃₁₋₁₄₅, Cap18₂₄₋₃₈, ADMTSL5₂₄₅₋₂₅₉, ADMTSL5₂₆₇₋₂₈₁, ADMTSL5₃₇₂₋₃₈₆, ADMTSL5₂₈₉₋₃₀₃, ADMTSL5₃₉₆₋₄₁₀, ADMTSL5₄₃₃₋₄₄₇, ADMTSL5₁₄₂₋₁₅₆, ADMTSL5₂₃₆₋₂₅₀, ADMTSL5₃₀₁₋₃₁₅, ADMTSL5₂₀₃₋₂₁₇, ADMTSL5₄₀₄₋₄₁₈, or a fragment or an equivalent of each thereof, and the MHC protein of the pMHC complex comprises all or part of HLA-DR or a fragment or an equivalent thereof;
m) an autoimmune hepatitis-relevant antigen derived from an antigen selected from one or more of the group: CYP2D6₁₉₃₋₂₀₇, CYP2D6₇₆₋₉₀, CYP2D6₂₉₃₋₃₀₇, CYP2D6₃₁₃₋₃₃₂, CYP2D6₃₉₃₋₄₁₂, CYP2D6₁₉₉₋₂₁₃, CYP2D6₄₅₀₋₄₆₄, CYP2D6₃₀₁₋₃₁₅, CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₃₁₋₁₄₅, CYP2D6₂₁₆₋₂₃₀, CYP2D6₂₃₈₋₂₅₂, CYP2D6₁₉₉₋₂₁₃, CYP2D6₂₃₅₋₂₅₂, CYP2D6₂₉₃₋₃₀₇, CYP2D6₃₈₁₋₃₉₅, CYP2D6₄₂₉₋₄₄₃, SLA₃₃₄₋₃₄₈, SLA₁₉₆₋₂₁₀, SLA₁₁₅₋₁₂₉, SLA₃₇₃₋₃₈₆, SLA₁₈₆₋₁₉₇, SLA₃₁₇₋₃₃₁, SLA₁₇₁₋₁₈₅, SLA₄₁₇₋₄₃₁, SLA₃₅₉₋₃₇₃, SLA₂₁₅₋₂₂₉, SLA₁₁₁₋₁₂₅, SLA₁₁₀₋₁₂₄, SLA₂₉₉₋₃₁₃, SLA₃₄₂₋₃₅₆, SLA₄₉₋₆₃, SLA₁₁₉₋₁₃₃, SLA₂₆₀₋₂₇₄, SLA₂₆₋₄₀, SLA₈₆₋₁₀₀, SLA₃₃₁₋₃₄₅, or a fragment or an equivalent of each thereof, and the MHC protein of the pMHC complex comprises all or part of HLA-DR or a fragment or an equivalent thereof;
n) an uveitis-relevant antigen derived from an antigen selected from one or more of the group: arrestin₁₉₉₋₂₁₃, arrestin₇₇₋₉₁, arrestin₂₅₀₋₂₆₄, arrestin₁₇₂₋₁₈₆, arrestin₃₅₄₋₃₆₈, arrestin₂₃₉₋₂₅₃, arrestin₁₀₂₋₁₁₆, arrestin₅₉₋₇₃, arrestin₂₈₀₋₂₉₄, arrestin₂₉₁₋₃₀₆, arrestin₁₉₅₋₂₀₉, arrestin₂₀₀₋₂₁₄, or a fragment or an equivalent of each thereof, and the MHC protein of the pMHC complex comprises all or part of HLA-DR or a fragment or an equivalent thereof;
o) a Sjogren's Syndrome-relevant antigen derived from an antigen selected from one or more of the group: RO60₁₂₇₋₁₄₁, RO60₅₂₃₋₅₃₇, RO60₂₄₃₋₂₅₇, RO60₄₈₄₋₄₉₈, RO60₃₄₇₋₃₆₁, RO60₃₆₉₋₃₈₃, RO60₄₂₆₋₄₄₀, RO60₂₆₇₋₂₈₁, RO60₁₇₈₋₁₉₂, RO60₃₅₈₋₃₇₂, RO60₂₂₁₋₂₃₅, RO60₃₁₈₋₃₃₂, RO60₄₀₇₋₄₂₁, RO60₄₅₉₋₄₇₃, RO60₅₁₋₆₅, RO60₃₁₂₋₃₂₆, LA₂₄₁₋₂₅₅, LA₁₀₁₋₁₁₅, LA₁₅₃₋₁₆₇, LA₁₇₈₋₁₉₂, LA₁₉₋₃₃, LA₃₇₋₅₁, LA₁₃₃₋₁₄₇, LA₅₀₋₆₄, LA₃₂₋₄₆, LA₁₅₃₋₁₆₇, LA₈₃₋₉₇, LA₁₃₆₋₁₅₀, LA₂₉₇₋₃₁₁, LA₅₉₋₇₃, LA₁₅₁₋₁₆₅, LA₈₆₋₁₀₀, LA₁₅₄₋₁₆₈, or a fragment or an equivalent of each thereof, and the MHC protein of the pMHC complex comprises all or part of a polypeptide of the group: HLA-DR, HLA-DP, or a fragment or an equivalent of each thereof;
p) a scleroderma-relevant antigen derived from an antigen selected from one or more of the group: TOP1₃₄₆₋₃₆₀, TOP1₄₂₀₋₄₃₄, TOP1₇₅₀₋₇₆₄, TOP1₄₁₉₋₄₃₃, TOP1₅₉₁₋₆₀₅, TOP1₆₉₅₋₇₀₉, TOP1₃₀₅₋₃₁₉, TOP1₃₄₆₋₃₆₀, TOP1₄₁₉₋₄₃₃, TOP1₄₂₅₋₄₃₉, TOP1₆₁₄₋₆₂₈, CENP-C₂₉₇₋₃₁₁, CENP-C₈₅₇₋₈₇₁, CENP-C₈₈₇₋₉₀₁, CENP-C₂₁₂₋₂₂₆, CENP-C₆₄₃₋₆₅₇, CENP-C₈₃₂₋₈₄₆, CENP-C₁₆₇₋₁₈₁, CENP-C₂₄₆₋₂₆₀, CENP-C₈₄₆₋₈₆₀, CENP-C₁₄₉₋₁₆₃, CENP-C₈₃₃₋₈₄₇, CENP-C₈₄₇₋₈₆₁, or a fragment or an equivalent of each thereof, and the MHC protein of the pMHC complex comprises all or part of HLA-DR or a fragment or an equivalent thereof;
q) an anti-phospholipid syndrome-relevant antigen derived from an antigen selected from one or more of the group: APOH₂₃₅₋₂₄₉, APOH₃₀₆₋₃₂₀, APOH₂₃₇₋₂₅₁, APOH₂₉₅₋₃₀₉, APOH₂₈₄₂, APOH₁₇₃₋₁₈₇, APOH₂₆₄₋₂₇₈, APOH₂₉₅₋₃₀₉, APOH₄₉₋₆₃, APOH₂₆₉₋₂₈₃, APOH₂₉₅₋₃₀₉, APOH₃₂₁₋₃₅₅, APOH₃₂₂₋₃₃₆, APOH₃₂₄₋₃₃₈, or a fragment or an equivalent of each thereof, and the MHC protein of the pMHC complex comprises all or part of HLA-DR or a fragment or an equivalent thereof;
r) an ANCA-associated vasculitis-relevant antigen derived from an antigen selected from one or more of the group: MPO₅₀₆₋₅₂₀, MPO₃₀₂₋₃₁₆, MPO₇₋₂₁, MPO₆₈₉₋₇₀₃, MPO₂₄₈₋₂₆₂, MPO₄₄₄₋₄₅₈, MPO₅₁₃₋₅₂₇, MPO₉₇₋₁₁₁, MPO₆₁₆₋₆₃₀, MPO₄₆₂₋₄₇₆, MPO₆₁₇₋₆₃₁, MPO₇₁₄₋₇₂₈, PRTN3₄₄₋₅₈, PRTN3₂₃₄₋₂₄₈, PRTN3₅₉₋₇₃, PRTN3₁₁₇₋₁₃₁, PRTN3₁₆₄₋₁₇₈, PRTN3₇₁₋₈₅, PRTN3₂₄₁₋₂₅₅, PRTN3₅₉₋₇₃, PRTN3₁₈₃₋₁₉₇, PRTN3₆₂₋₇₆, PRTN3₁₁₈₋₁₃₂, PRTN3₂₃₉₋₂₅₃, or a fragment or an equivalent of each thereof, and the MHC protein of the pMHC complex comprises all or part of HLA-DR or a fragment or an equivalent thereof; or
s) a Stiff Man Syndrome-relevant antigen derived from an antigen selected from one or more of the group: GAD₂₁₂₋₂₂₆, GAD₅₅₅₋₅₆₉, GAD₂₉₇₋₃₁₁, and the MHC protein of the pMHC complex comprises all or part of a polypeptide of the group: HLA-DR, HLA-DQ, or a fragment or an equivalent of each thereof.

In certain aspects, the pMHC complex is for the treatment of:
a) type I diabetes and the pMHC complex is selected from the group of: PPI_{76-90(K88S)}-HLA-DRB1*0401/DRA, IGRP₁₃₋₂₅-HLA-DRB1*0301/DRA, GAD₅₅₅₋₅₆₇-HLA-DRB1*0401/DRA, GAD_{555-567(557I)}-HLA-DRB1*0401/DRA, IGRP₂₃₋₃₅-HLA-DRB1*0401/DRA, B₂₄-C₃₆-HLA-DRB1*0301/DRA, or PPI₇₆₋₉₀-HLA-DRB1*0401/DRA;
b) multiple sclerosis and the pMHC complex is selected from the group of: MBP₈₆₋98-HLA-DRB1*1501/DRA, MBP₈₉₋₁₀₁-HLA-DRB5*0101/DRA, MOG₃₈₋₅₂-HLA-DRB4*0101/DRA, MOG_{97-109(E107S)}-HLA-DRB1*0401/DRA, MOG₂₀₃₋₂₁₇-HLA-DRB3*0101/DRA, PLP₅₄₋₆₅-HLA-DRB3*0101/DRA, PLP₉₄₋₁₀₈-HLA-DRB1*0301/DRA, PLP₂₅₀₋₂₆₄-HLA-DRB4*0101/DRA, MPB₁₃₋₃₂-HLA-DRB5*0101/DRA, MPB₈₃₋₉₉-HLA-DRB5*0101/DRA, MPB₁₁₁₋₁₂₉-HLA-DRB5*0101/DRA, MPB₁₄₆₋₁₇₀-HLA-DRB5*0101/DRA, MOG₂₂₃₋₂₃₇-HLA-DRB3*0202/DRA, MOG₆₋₂₀-HLA-DRB5*0101/DRA, PLP₈₈₋₁₀₂-HLA-DRB3*0202/DRA, or PLP₁₃₉₋₁₅₄-HLA-DRB5*0101/DRA;
c) Celiac Disease and the pMHC complex is selected from the group of: aGlia₅₇₋₆₈-HLA-DQA1*0501/HLA-DQB1*0201, aGlia₆₂₋₇₂-HLA-DQA1*0501/HLA-DQB1*0201, aGlia₂₁₇₋₂₂₉-HLA-DQA1*0501/HLA-DQB1*0302, or aGlia₂₁₇₋₂₂₉-HLA-DQA1*03/HLA-DQB1*0302;
d) primary biliary cirrhosis and the pMHC complex is selected from the group of: PDC-E2₁₂₂₋₁₃₅-HLA-DRB4*0101/DRA, PDC-E2₂₄₉₋₂₆₂-HLA-DRB4*0101/DRA, PDC-E2₂₄₉₋₂₆₃-HLA-DRB1*0801/DRA, PDC-E2₆₂₉₋₆₄₃-HLA-DRB1*0801/DRA, PDC-E2₇₂₋₈₆-HLA-DRB3*0202/DRA, PDC-E2₅₃₋₃₆₇-HLA-DRB3*0202/DRA, PDC-E2₄₂₂₋₄₃₆-HLA-DRB3*0202/DRA, PDC-E2₆₂₉₋₆₄₃-HLA-DRB4*0101/DRA, PDC-E2₈₀₋₉₄-HLA-DRB5*0101/DRA, PDC-E2₅₃₋₃₆₇-HLA-DRB5*0101/DRA, or PDC-E2₅₃₅₋₅₄₉-HLA-DRB5*0101/DRA, mPDC-E2₁₆₆₋₁₈₁-I-A_{g7}, or mPDC-E2₈₂₋₉₆-I-A_{g7};
e) pemphigus folliaceus and/or pemphigus vulgaris and the pMHC complex is selected from the group of: DG1₂₁₆₋₂₂₉-HLA-DRB1*0101/DRA, DG1₂₁₆₋₂₂₉-HLA-DRB1*0102/DRA, DG3₉₇₋₁₁₁-HLA-DRB1*0402/DRA, DG3₂₅₁₋₂₆₅-HLA-DRB1*0402/DRA, DG3₂₅₁₋₂₆₅-HLA-DRB1*0401/DRA, DG3₄₄₁₋₄₅₅-HLA-DRB1*0402/DRA, DG3₃₅₁₋₃₆₅-HLA-DRB3*0202/DRA, DG3₄₅₃₋₄₆₇-HLA-DRB3*0202/DRA, DG3₅₄₀₋₅₅₄-HLA-DRB3*0202/DRA, DG3₂₈₀₋₂₉₄-HLA-DRB4*0101/DRA, DG3₃₂₆₋₃₄₀-HLA-DRB4*0101/DRA, DG3₃₆₇₋₃₈₁-HLA-DRB4*0101/DRA, DG3₁₃₋₂₇-HLA-DRB5*0101/DRA, DG3₃₂₃₋₃₃₇-HLA-DRB5*0101/DRA, DG3₄₃₈₋₄₅₂-HLA-DRB5*0101/DRA, DG1₄₈₋₆₂-HLA-DRB3*0202/DRA, DG1₂₀₆₋₂₂₂-HLA-DRB3*0202/DRA, DG1₃₆₃₋₃₇₇-HLA-DRB3*0202/DRA, DG1₃₋₁₇-HLA-DRB4*0101/DRA, DG1₁₉₂₋₂₀₆-HLA-DRB4*0101/DRA, DG1₃₂₆₋₃₄₀-HLA-DRB4*0101/DRA, DG1₁₋₁₅-HLA-DRB5*0101/DRA, DG1₃₅₋₄₉-HLA-DRB5*0101/DRA, or DG1₃₂₅₋₃₃₉-HLA-DRB5*0101/DRA;
f) neuromyelitis optica spectrum disorder and the pMHC complex is selected from the group of: AQP4₁₂₉₋₁₄₃-HLA-DRB1*0101/DRA, AQP4₂₈₄₋₂₉₈-HLA-DRB1*0301/DRA, AQP4₆₃₋₇₆-HLA-DRB1*0301/DRA, AQP4₁₂₉₋₁₄₃-HLA-DRB1*0401/DRA, or AQP4₃₉₋₅₃-HLA-DRB1*1501/DRA;
g) allergic asthma and the pMHC complex is selected from the group of: DERP-1₁₆₋₃₀-HLA-DRB1*0101/DRA, DERP-1₁₆₋₃₀-HLA-DRB1*1501/DRA, DERP₁₁₇₁₋₁₈₅-HLA-DRB1*1501/DRA, DERP-1₁₁₀₋₁₂₄-HLA-DPB1*0401/DRA, DERP-2₂₆₋₄₀-HLA-DRB1*0101/DRA; DERP-2₂₆₋₄₀-HLA-DRB1*1501/DRA, or DERP-2₁₀₇₋₁₂₁-HLA-DRB1*0301/DRA;
h) inflammatory bowel disease and the pMHC complex is selected from the group of: bacteroides integrase antigen₁₈₃₋₁₉₇-HLA-DRB3*0101/DRA, bacteroides integrase antigen₁₄₆₋₁₆₀-HLA-DRB3*0101/DRA, bacteroides integrase antigen₁₇₅₋₁₈₉-HLA-DRB3*0101/DRA, bacteroides integrase antigen₁₋₁₅-HLA-DRB5*0101/DRA, bacteroides integrase antigen₁₈₃₋₁₉₇-HLA-DRB5*0101/DRA, bacteroides integrase antigen₁₈₃₋₁₉₇-HLA-DRB3*0101/DRA, bacteroides integrase antigen₃₀₋₄₄-HLA-DRB5*0101/DRA, bacteroides integrase antigen₇₀₋₈₄-HLA-DRB4*0101/DRA, bacteroides integrase antigen₃₃₇₋₃₅₁-HLA-DRB4*0101/DRA, bacteroides integrase antigen₁₇₁₋₁₈₅-HLA-DRB4*0101/DRA, bacteroides integrase antigen₄-is-HLA-DRB3*0202/DRA, bacteroides integrase antigen₁₇₁₋₁₈₅-HLA-DRB3*0202/DRA, bacteroides integrase antigen₂₅₆₋₂₇₀-HLA-DRB3*0202/DRA, Fla-2/Fla-X₃₆₆₋₃₈₀-HLA-DRB3*0101/DRA, Fla-2/Fla-X₁₆₄₋₁₇₈-HLA-DRB3*0101/DRA, Fla-2/Fla-X₂₆₁₋₂₇₅-HLA-DRB5*0101/DRA, Fla-2/Fla-X₁₋₁₅-HLA-DRB5*0101/DRA, Fla-2/Fla-X₅₁₋₆₅-HLA-DRB4*0101/DRA, Fla-2/Fla-X₂₆₉₋₂₈₃-HLA-DRB4*0101/DRA, Fla-2/Fla-X₄₋₁₈-HLA-DRB3*0202/DRA, Fla-2/Fla-X₂₆₁₋₂₇₅-HLA-DRB3*0202/DRA, Fla-2/Fla-X₂₇₁₋₂₈₅-HLA-DRB3*0202/DRA, YIDX₇₈₋₉₂-HLA-DRB3*0101/DRA, YIDX₇₈₋₉₂-HLA-DRB4*0101/DRA, YIDX₉₃₋₁₀₇-HLA-DRB3*0101/DRA, YIDX₉₈₋₁₁₂-HLA-DRB5*0101/DRA, YIDX₂₃₋₃₇-HLA-DRB5*0101/DRA, YIDX₇₈₋₉₂-HLA-DRB4*0101/DRA, YIDX₁₉₅₋₂₀₉-HLA-DRB4*0101/DRA, YIDX₂₂₋₃₆-HLA-DRB3*0202/DRA, YIDX₈₀₋₉₄-HLA-DRB3*0202/DRA, or YIDX₁₀₁₋₁₁₅-HLA-DRB3*0202/DRA;
i) COPD and/or emphysema and the pMHC complex is selected from the group of: elastin₈₉₋₁₀₃-HLA-DRB3*0101/DRA, elastin₆₉₈₋₇₁₂-HLA-DRB5*0101/DRA, elastin₈₋₂₂-HLA-DRB5*0101/DRA, elastin₉₄₋₁₀₈-HLA-DRB5*0101/DRA, elastin₁₃₋₂₇-HLA-DRB4*0101/DRA, elastin₆₉₅₋₇₀₉-HLA-DRB4*0101/DRA, elastin₅₆₃₋₅₇₇-HLA-DRB4*0101/DRA, elastin₅₅₈₋₅₇₂-HLA-DRB4*0101/DRA, elastin₆₉₈₋₇₁₂-HLA-DRB5*0101/DRA, elastin₅₆₆₋₅₈₀-HLA-DRB3*0202/DRA, or elastin₅₄₅₋₆₅₉-HLA-DRB3*0202/DRA;
j) psoriasis and the pMHC complex is selected from the group of: Cap18₆₄₋₇₈-HLA-DRB3*0101/DRA, Cap18₃₄₋₄₈-HLA-DRB3*0101/DRA, Cap18₄₇₋₆₁-HLA-DRB3*0101/DRA, Cap18₁₅₁₋₁₆₅-HLA-DRB4*0101/DRA, Cap18₁₄₉₋₁₆₃-HLA-DRB5*0101/DRA, Cap18₁₅₂₋₁₆₆-HLA-DRB5*0101/DRA, Cap18₁₃₁₋₁₄₅-HLA-DRB5*0101/DRA, Cap18₂₄₋₃₈-HLA-DRB3*0202/DRA, ADMTSL5₂₄₅₋₂₅₉-HLA-DRB3*0101/DRA, ADMTSL5₂₆₇₋₂₈₁-HLA-DRB3*0101/DRA, ADMTSL5₃₇₂₋₃₈₆-HLA-DRB3*0101/DRA, ADMTSL5₂₈₉₋₃₀₃-HLA-DRB4*0101/DRA, ADMTSL5₃₉₆₋₄₁₀-HLA-DRB4*0101/DRA, ADMTSL5₄₃₃₋₄₄₇-HLA-DRB4*0101/DRA, ADMTSL5₁₄₂₋₁₅₆-HLA-DRB5*0101/DRA, ADMTSL5₂₃₆₋₂₅₀-HLA-DRB5*0101/DRA, ADMTSL5₃₀₁₋₃₁₅-HLA-DRB5*0101/DRA, ADMTSL5₂₀₃₋₂₁₇-HLA-DRB3*0202/DRA, ADMTSL5₄₀₄₋₄₁₈-HLA-DRB3*0202/DRA, or ADMTSL5₄₃₃₋₄₄₇-HLA-DRB3*0202/DRA;
k) autoimmune hepatitis and the pMHC complex is selected from the group of: CYP2D6₁₉₃₋₂₀₇-HLA-DRB1*0301/DRA, CYP2D6₇₆₋₉₀-HLA-DRB1*0301/DRA, CYP2D6₂₉₃₋₃₀₇-HLA-DRB1*0301/DRA, CYP2D6₃₁₃₋₃₃₂-HLA-DRB1*0301/DRA, CYP2D6₃₉₃₋₄₁₂-HLA-DRB1*0301/DRA, CYP2D6₁₉₉₋₂₁₃-HLA-DRB1*0401/DRA, CYP2D6₄₅₀₋₄₆₄-HLA-DRB1*0401/DRA, CYP2D6₃₀₁₋₃₁₅-HLA-DRB1*0401/DRA, CYP2D6₄₅₂₋₄₆₆-HLA-DRB1*0701/DRA, CYP2D6₅₉₋₇₃-HLA-DRB1*0701/DRA, CYP2D6₁₃₀₋₁₄₄-HLA-DRB1*0701/DRA, CYP2D6₁₉₃₋₂₁₂-HLA-DRB1*0701/DRA, CYP2D6₃₀₅₋₃₂₄-HLA-DRB1*0701/DRA, CYP2D6₁₃₁₋₁₄₅-HLA-DRB3*0202/DRA, CYP2D6₂₁₆₋₂₃₀-HLA-DRB3*0202/DRA, CYP2D6₂₃₈₋₂₅₂-HLA-DRB3*0202/DRA, CYP2D6₁₉₉₋₂₁₃-HLA-DRB4*0101/DRA, CYP2D6₂₃₅₋₂₅₂-HLA-DRB4*0101/DRA, CYP2D6₂₉₃₋₃₀₇-HLA-DRB4*0101/DRA, CYP2D6₂₃₈₋₂₅₂-HLA-DRB5*0101/DRA, CYP2D6₃₈₁₋₃₉₅-HLA-DRB5*0101/DRA, CYP2D6₄₂₉₋₄₄₃-HLA-DRB5*0101/DRA, SLA₃₃₄₋₃₄₈-HLA-DRB1*0301/DRA, SLA₁₉₆₋₂₁₀-HLA-DRB1*0301/DRA, SLA₁₁₅₋₁₂₉-HLA-DRB1*0301/DRA, SLA₃₇₃₋₃₈₆-HLA-DRB1*0301/DRA, SLA₁₈₆₋₁₉₇-HLA-DRB1*0301/DRA, SLA₃₁₇₋₃₃₁-HLA-DRB1*0401/DRA, SLA₁₇₁₋₁₈₅-HLA-DRB1*0401/DRA, SLA₄₁₇₋₄₃₁-HLA-DRB1*0401/DRA, SLA₃₅₉₋₃₇₃-HLA-DRB1*0701/DRA, SLA₂₁₅₋₂₂₉-HLA-DRB1*0701/DRA, SLA₁₁₁₋₁₂₅-HLA-DRB1*0701/DRA, SLA₁₁₀₋₁₂₄-HLA-DRB3*0202/DRA, SLA₂₉₉₋₃₁₃-HLA-DRB3*0202/DRA, SLA₃₄₂₋₃₅₆-HLA-DRB3*0202/DRA, SLA₄₉₋₆₃-HLA-DRB4*0101/DRA, SLA₁₁₉₋₁₃₃-HLA-DRB4*0101/DRA, SLA₂₆₀₋₂₇₄-HLA-DRB4*0101/DRA, SLA₂₆₋₄₀-HLA-DRB5*0101/DRA, SLA₈₆₋₁₀₀-HLA-DRB5*0101/DRA, or SLA₃₃₁₋₃₄₅-HLA-DRB5*0101/DRA;
l) uveitis and the pMHC complex is selected from the group of: arrestin₁₉₉₋₂₁₃-HLA-DRB3*0101/DRA, arrestin₇₇₋₉₁-HLA-DRB3*0101/DRA, arrestin₂₅₀₋₂₆₄-HLA-DRB3*0101/DRA, arrestin₁₇₂₋₁₈₆-HLA-DRB4*0101/DRA, arrestin₃₅₄₋₃₆₈-HLA-DRB4*0101/DRA, arrestin₂₃₉₋₂₅₃-HLA-DRB4*0101/DRA, arrestin₁₀₂₋₁₁₆-HLA-DRB5*0101/DRA, arrestin₅₉₋₇₃-HLA-DRB5*0101, arrestin₂₈₀₋₂₉₄-HLA-DRB5*0101, arrestin₂₉₁₋₃₀₆-HLA-DRB1*0301/DRA, arrestin₁₉₅₋₂₀₉-HLA-DRB3*0202/DRA, arrestin₁₉₉₋₂₁₃-HLA-DRB3*0202/DRA, or arrestin₂₀₀₋₂₁₄-HLA-DRB3*0202/DRA;
m) Sjogren Syndrome and the pMHC complex is selected from the group of: RO60₁₂₇₋₁₄₁-HLA-DRB1*0301/DRA, RO60₅₂₃₋₅₃₇-HLA-DRB1*0301/DRA, RO60₂₄₃₋₂₅₇-HLA-DRB1*0301/DRA, RO60₄₈₄₋₄₉₈-HLA-DRB3*0101/DRA, RO60₃₄₇₋₃₆₁-HLA-DRB3*0101/DRA, RO60₃₆₉₋₃₈₃-HLA-DRB3*0101/DRA, RO60₄₂₆₋₄₄₀-HLA-DRB4*0101/DRA, RO60₂₆₇₋₂₈₁-HLA-DRB4*0101/DRA, RO60₁₇₈₋₁₉₂-HLA-DRB4*0101/DRA, RO60₃₅₈₋₃₇₂-HLA-DRB5*0101/DRA, RO60₃₅₈₋₃₇₂-HLA-DRB4*0101/DRA, RO60₂₂₁₋₂₃₅-HLA-DRB5*0101/DRA, RO60₂₂₁₋₂₃₅-HLA-DRB4*0101/DRA, RO60₃₁₈₋₃₃₂-HLA-DRB5*0101/DRA, RO60₃₁₈₋₃₃₂-HLA-DRB4*0101/DRA, RO60₄₀₇₋₄₂₁-HLA-DRB4*0101/DRA, RO60₄₀₇₋₄₂₁-HLA-DQA1*0501/HLA-DQB1*0201, RO60₄₅₉₋₄₇₃-HLA-DRB4*0101/DRA, RO60₄₅₉₋₄₇₃-HLA-DQA1*0501/HLA-DQB1*0201, RO60₃₁₈₋₃₃₂-HLA-DQA1*0501/HLA-DQB1*0201, RO60₅₁₋₆₅-HLA-DRB3*0202/DRA, RO60₃₁₂₋₃₂₆-HLA-DRB3*0202/DRA, RO60₃₄₇₋₃₆₁-HLA-DRB3*0202/DRA, LA₂₄₁₋₂₅₅-HLA-DRB1*0301/DRA, LA₁₀₁₋₁₁₅-HLA-DRB1*0301/DRA, LA₁₅₃₋₁₆₇-HLA-DRB1*0301/DRA, LA₁₇₈₋₁₉₂-HLA-DRB3*0101/DRA, LA₁₉₋₃₃-HLA-DRB3*0101/DRA, LA₃₇₋₅₁-HLA-DRB3*0101/DRA, LA₁₃₃₋₁₄₇-HLA-DRB4*0101/DRA, LA₅₀₋₆₄-HLA-DRB4*0101/DRA, LA₃₂₄₆-HLA-DRB4*0101/DRA, LA₁₅₃₋₁₆₇-HLA-DRB5*0101/DRA, LA₈₃₋₉₇-HLA-DRB5*0101/DRA, LA₁₃₆₋₁₅₀-HLA-DRB5*0101/DRA, LA₂₉₇₋₃₁₁-HLA-DQA1*0501/HLA-DQB1*0201, LA₅₉₋₇₃-HLA-DQA1*0501/HLA-DQB1*0201, LA₅₉₋₇₃-HLA-DRB4*0101/DRA, LA₁₅₁₋₁₆₅-HLA-DQA1*0501/HLA-DQB1*0201, LA₁₅₁₋₁₆₅-HLA-DRB4*0101/DRA, LA₂₉₇₋₃₁₁-HLA-DRB4*0101/DRA, LA₅₀₋₆₄-HLA-DRB3*0202/DRA, LA₈₆₋₁₀₀-HLA-DRB3*0202/DRA, or LA₁₅₄₋₁₆₈-HLA-DRB3*0202/DRA;
n) scleroderma and the pMHC complex is selected from the group of: TOP1₃₄₆₋₃₆₀-HLA-DRB3*0101/DRA, TOP1₄₂₀₋₄₃₄-HLA-DRB3*0101/DRA, TOP1₇₅₀₋₇₆₄-HLA-DRB3*0101/DRA, TOP1₄₁₉₋₄₃₃-HLA-DRB4*0101/DRA, TOP1₅₉₁₋₆₀₅-HLA-DRB4*0101/DRA, TOP1₆₉₅₋₇₀₉-HLA-DRB4*0101/DRA, TOP1₃₀₅₋₃₁₉-HLA-DRB5*0101/DRA, TOP1₃₄₆₋₃₆₀-HLA-DRB5*0101/DRA, TOP1₄₁₉₋₄₃₃-HLA-DRB5*0101/DRA, TOP1₄₂₀₋₄₃₄-HLA-DRB3*0202/DRA, TOP1₄₂₅₋₄₃₉-HLA-DRB3*0202/DRA, TOP1₆₁₄₋₆₂₈-HLA-DRB3*0202/DRA, CENP-C₂₉₇₋₃₁₁-HLA-DRB3*0101/DRA, CENP-C₈₅₇₋₈₇₁-HLA-DRB3*0101/DRA, CENP-C₈₈₇₋₉₀₁-HLA-DRB3*0101/DRA, CENP-C₂₁₂₋₂₂₆-HLA-DRB4*0101/DRA, CENP-C₆₄₃₋₆₅₇-HLA-DRB4*0101/DRA, CENP-C₈₃₂₋₈₄₆-HLA-DRB4*0101/DRA, CENP-C₁₆₇₋₁₈₁-HLA-DRB5*0101/DRA, CENP-C₂₄₆₋₂₆₀-HLA-DRB5*0101/DRA, CENP-C₈₄₆₋₈₆₀-HLA-DRB5*0101/DRA, CENP-C₁₄₉₋₁₆₃-HLA-DRB3*0202/DRA, CENP-C₈₃₃₋₈₄₇-HLA-DRB3*0202/DRA, or CENP-C₈₄₇₋₈₆₁-HLA-DRB3*0202/DRA;
o) anti-phospholipid syndrome and the pMHC complex is selected from the group of: APOH₂₃₅₋₂₄₉-HLA-DRB3*0101/DRA, APOH₃₀₆₋₃₂₀-HLA-DRB3*0101/DRA, APOH₂₃₇₋₂₅₁-HLA-DRB3*0101/DRA, APOH₂₉₅₋₃₀₉-HLA-DRB3*0101/DRA, APOH₂₈₋₄₂-HLA-DRB4*0101/DRA, APOH₁₇₃₋₁₈₇-HLA-DRB4*0101/DRA, APOH₂₆₄₋₂₇₈-HLA-DRB4*0101/DRA, APOH₂₉₅₋₃₀₉-HLA-DRB4*0101/DRA, APOH₄₉₋₆₃-HLA-DRB5*0101/DRA, APOH₂₆₉₋₂₈₃-HLA-DRB5*0101/DRA, APOH₂₉₅₋₃₀₉-HLA-DRB5*0101/DRA, APOH₃₂₁₋₃₅₅-HLA-DRB3*0202/DRA, APOH₃₂₂₋₃₃₆-HLA-DRB3*0202/DRA, or APOH₃₂₄₋₃₃₈-HLA-DRB3*0202/DRA;
p) ANCA-associated vasculitis and the pMHC complex is selected from the group of: MPO₅₀₆₋₅₂₀-HLA-DRB3*0101/DRA, MPO₃₀₂₋₃₁₆-HLA-DRB3*0101/DRA, MPO₇₋₂₁-HLA-DRB3*0101/DRA, MPO₆₈₉₋₇₀₃-HLA-DRB4*0101/DRA, MPO₂₄₈₋₂₆₂-HLA-DRB4*0101/DRA, MPO₄₄₄₋₄₅₈-HLA-DRB4*0101/DRA, MPO₅₁₃₋₅₂₇-HLA-DRB5*0101/DRA, MPO₉₇₋₁₁₁-HLA-DRB5*0101/DRA, MPO₆₁₆₋₆₃₀-HLA-DRB5*0101/DRA, MPO₄₆₂₋₄₇₆-HLA-DRB3*0202/DRA, MPO₆₁₇₋₆₃₁-HLA-DRB3*0202/DRA, MPO₇₁₄₋₇₂₈-HLA-DRB3*0202/DRA, PRTN3₄₄₋₅₈-HLA-DRB3*0101/DRA, PRTN3₂₃₄₋₂₄₈-HLA-DRB3*0101/DRA, PRTN3₅₉₋₇₃-HLA DRB3*0101/DRA, PRTN3₅₉₋₇₃-HLA-DRB5*0101/DRA, PRTN3₁₁₇₋₁₃₁-HLA-DRB4*0101/DRA, PRTN3₁₆₄₋₁₇₈-HLA-DRB4*0101/DRA, PRTN3₇₁₋₈₅-HLA-DRB4*0101/DRA, PRTN3₂₄₁₋₂₅₅-HLA-DRB5*0101/DRA, PRTN3₁₈₃₋₁₉₇-HLA-DRB5*0101/DRA, PRTN3₆₂₋₇₆-HLA-DRB3*0202/DRA, PRTN3₁₁₈₋₁₃₂-HLA-DRB3*0202/DRA, or PRTN3₂₃₉₋₂₅₃-HLA-DRB3*0202/DRA; or
q) Stiff Man Syndrome and the pMHC complex is selected from the group of: GAD₂₁₂₋₂₂₆-HLA-DRB1*0801/DRA, GAD₅₅₅₋₅₆₉-HLA-DRB1*0801/DRA, or GAD₂₉₇₋₃₁₁-HLA-DRB1*0301/DRA.

In some aspects, the pMHC complex is for the treatment of:
a) type I diabetes and the pMHC complex is selected from the group of: PPI_{76-90(K88S)}-HLA-DRB1*0401/DRA, IGRP₁₃₋₂₅-HLA-DRB1*0301/DRA, GAD₅₅₅₋₅₆₇-HLA-DRB1*0401/DRA, GAD_{555-567(557I)}-HLA-DRB1*0401/DRA, IGRP₂₃₋₃₅-HLA-DRB1*0401/DRA, or PPI₇₆₋₉₀-HLA-DRB1*0401/DRA;
b) multiple sclerosis and the pMHC complex is selected from the group of: MBP₈₆₋₉₈-HLA-DRB1*1501/DRA, MBP₈₉₋₁₀₁-HLA-DRB5*0101/DRA, MOG₃₈₋₅₂-HLA-DRB4*0101/DRA, MOG_{97-109(E107S)}-HLA-DRB1*0401/DRA, MOG₂₀₃₋₂₁₇-HLA-DRB3*0101/DRA, PLP₅₄₋₆₈-HLA-DRB3*0101/DRA, PLP₉₄₋₁₀₈-HLA-DRB1*0301/DRA, PLP₂₅₀₋₂₆₄-HLA-DRB4*0101/DRA, MPB₁₃₋₃₂-HLA-DRB5*0101/DRA, MPB₈₃₋₉₉-HLA-DRB5*0101/DRA, MPB₁₁₁₋₁₂₉-HLA-DRB5*0101/DRA, MPB₁₄₆₋₁₇₀-HLA-DRB5*0101/DRA, MOG₂₂₃₋₂₃₇-HLA-DRB3*0202/DRA, MOG₆₋₂₀-HLA-DRB5*0101/DRA, PLP₈₈₋₁₀₂-HLA-DRB3*0202/DRA, or PLP₁₃₉₋₁₅₄-HLA-DRB5*0101/DRA;
c) Celiac Disease and the pMHC complex is selected from the group of: aGlia₅₇₋₆₈-HLA-DQA1*0501/HLA-DQB1*0201, aGlia₆₂₋₇₂-HLA-DQA1*0501/HLA-DQB1*0201, or aGlia₂₁₇₋₂₂₉-HLA-DQA1*0501/HLA-DQB1*0302;
d) primary biliary cirrhosis and the pMHC complex is selected from the group of: PDC-E2₁₂₂₋₁₃₅-HLA-DRB4*0101/DRA, PDC-E2₂₄₉₋₂₆₂-HLA-DRB4*0101/DRA, PDC-E2₂₄₉₋₂₆₃-HLA-DRB1*0801/DRA, PDC-E2₆₂₉₋₆₄₃-HLA-DRB1*0801/DRA, PDC-E2₇₂₋₈₆-HLA-DRB3*0202/DRA, PDC-E2₃₅₃₋₃₆₇-HLA-DRB3*0202/DRA, PDC-E2₄₂₂₋₄₃₆-HLA-DRB3*0202/DRA, PDC-E2₆₂₉₋₆₄₃-HLA-DRB4*0101/DRA, PDC-E2₈₀₋₉₄-HLA-DRB5*0101/DRA, PDC-E2₃₅₃₋₃₆₇-HLA-DRB5*0101/DRA, or PDC-E2₅₃₅₋₅₄₉-HLA-DRB5*0101/DRA;
e) pemphigus folliaceus and/or pemphigus vulgaris and the pMHC complex is selected from the group of: DG1₂₁₆₋₂₂₉-HLA-DRB1*0101/DRA, DG3₉₇₋₁₁₁-HLA-DRB1*0402/DRA, DG3₂₅₁₋₂₆₅-HLA-DRB1*0401/DRA, DG3₄₄₁₋₄₅₅-HLA-DRB1*0402/DRA, DG3₃₅₁₋₃₆₅-HLA-DRB3*0202/DRA, DG3₄₅₃₋₄₆₇-HLA-DRB3*0202/DRA, DG3₅₄₀₋₅₅₄-HLA-DRB3*0202/DRA, DG3₂₈₀₋₂₉₄-HLA-DRB4*0101/DRA, DG3₃₂₆₋₃₄₀-HLA-DRB4*0101/DRA, DG3₃₆₇₋₃₈₁-HLA-DRB4*0101/DRA, DG3₁₃₋₂₇-HLA-DRB5*0101/DRA, DG3₃₂₃₋₃₃₇-HLA-DRB5*0101/DRA, DG3₄₃₈₋₄₅₂-HLA-DRB5*0101/DRA, DG1₄₈₋₆₂-HLA-DRB3*0202/DRA, DG1₂₀₆₋₂₂₂-HLA-DRB3*0202/DRA, DG1₃₆₃₋₃₇₇-HLA-DRB3*0202/DRA, DG1₃₋₁₇-HLA-DRB4*0101/DRA, DG1₁₉₂₋₂₀₆-HLA-DRB4*0101/DRA, DG1₃₂₆₋₃₄₀-HLA-DRB4*0101/DRA, DG1₁₋₁₅-HLA-DRB5*0101/DRA, DG1₃₅₋₄₉-HLA-DRB5*0101/DRA, or DG1₃₂₅₋₃₃₉-HLA-DRB5*0101/DRA;
f) neuromyelitis optica spectrum disorder and the pMHC complex is selected from the group of: AQP4₂₈₄₋₂₉₈-HLA-DRB1*0301/DRA, AQP4₆₃₋₇₆-HLA-DRB1*0301/DRA, AQP4₁₂₉₋₁₄₃-HLA-DRB1*0401/DRA, or AQP4₃₉₋₅₃-HLA-DRB1*1501/DRA;
g) allergic asthma and the pMHC complex is selected from the group of: DERP-1₁₆₋₃₀-HLA-DRB1*0101/DRA, DERP-1₁₆₋₃₀-HLA-DRB1*1501/DRA, DERP1₁₇₁₋₁₈₅-HLA-DRB1*1501/DRA, DERP-1₁₁₀₋₁₂₄-HLA-DPB1*0401/DRA, DERP-2₂₆₋₄₀-HLA-DRB1*0101/DRA; DERP-2₂₆₋₄₀-HLA-DRB1*1501/DRA, or DERP-2₁₀₇₋₁₂₁-HLA-DRB1*0301/DRA;
h) inflammatory bowel disease and the pMHC complex is selected from the group of: bacteroides integrase antigen₁₋₁₅-HLA-DRB5*0101/DRA, bacteroides integrase antigen₁₈₃₋₁₉₇-HLA-DRB3*0101/DRA, bacteroides integrase antigen₇₀₋₈₄-HLA-DRB4*0101/DRA, bacteroides integrase antigen₄₋₁₈-HLA-DRB3*0202/DRA, bacteroides integrase antigen₁₇₁₋₁₈₅-HLA-DRB3*0202/DRA, bacteroides integrase antigen₂₅₆₋₂₇₀-HLA-DRB3*0202/DRA, Fla-2/Fla-X₃₆₆₋₃₈₀-HLA-DRB3*0101/DRA, Fla-2/Fla-X₂₆₁₋₂₇₅-HLA-DRB5*0101/DRA, Fla-2/Fla-X₅₁₋₆₅-HLA-DRB4*0101/DRA, Fla-2/Fla-X₄₋₁₈-HLA-DRB3*0202/DRA, Fla-2/Fla-X₂₆₁₋₂₇₅-HLA-DRB3*0202/DRA, Fla-2/Fla-X₂₇₁₋₂₈₅-HLA-DRB3*0202/DRA, YIDX₇₈₋₉₂-HLA-DRB3*0101/DRA, YIDX₇₈₋₉₂-HLA-DRB4*0101/DRA, YIDX₉₈₋₁₁₂-HLA-DRB5*0101/DRA, YIDX₂₂₋₃₆-HLA-DRB3*0202/DRA, YIDX₈₀₋₉₄-HLA-DRB3*0202/DRA, or YIDX₁₀₁₋₁₁₅-HLA-DRB3*0202/DRA;
i) emphysema and the pMHC complex is selected from the group of: elastin₈₉₋₁₀₃-HLA-DRB3*0101/DRA, elastin₆₉₈₋₇₁₂-HLA-DRB5*0101/DRA, elastin₅₅₈₋₅₇₂-HLA-DRB4*0101/DRA, elastin₅₆₆₋₅₈₀-HLA-DRB3*0202/DRA, or elastin₅₄₅₋₆₅₉-HLA-DRB3*0202/DRA;
j) psoriasis and the pMHC complex is selected from the group of: Cap18₆₄₋₇₈-HLA-DRB3*0101/DRA, Cap18₃₄₋₄₈-HLA-DRB3*0101/DRA, Cap18₄₇₋₆₁-HLA-DRB3*0101/DRA, Cap18₁₅₁₋₁₆₅-HLA-DRB4*0101/DRA, Cap18₁₄₉₋₁₆₃-HLA-DRB5*0101/DRA, Cap18₁₅₂₋₁₆₆-HLA-DRB5*0101/DRA, Cap18₁₃₁₋₁₄₅-HLA-DRB5*0101/DRA, Cap18₂₄₋₃₈-HLA-DRB3*0202/DRA, ADMTSL5₂₄₅₋₂₅₉-HLA-DRB3*0101/DRA, ADMTSL5₂₆₇₋₂₈₁-HLA-DRB3*0101/DRA, ADMTSL5₃₇₂₋₃₈₆-HLA-DRB3*0101/DRA, ADMTSL5₂₈₉₋₃₀₃-HLA-DRB4*0101/DRA, ADMTSL5₃₉₆₋₄₁₀-HLA-DRB4*0101/DRA, ADMTSL5₄₃₃₋₄₄₇-HLA-DRB4*0101/DRA, ADMTSL5₁₄₂₋₁₅₆-HLA-DRB5*0101/DRA, ADMTSL5₂₃₆₋₂₅₀-HLA-DRB5*0101/DRA, ADMTSL5₃₀₁₋₃₁₅-HLA-DRB5*0101/DRA, ADMTSL5₂₀₃₋₂₁₇-HLA-DRB3*0202/DRA, ADMTSL5₄₀₄₋₄₁₈-HLA-DRB3*0202/DRA, or ADMTSL5₄₃₃₋₄₄₇-HLA-DRB3*0202/DRA;
k) autoimmune hepatitis and the pMHC complex is selected from the group of: CYP2D6₁₉₃₋₂₀₇-HLA-DRB1*0301/DRA, CYP2D6₇₆₋₉₀-HLA-DRB1*0301/DRA, CYP2D6₂₉₃₋₃₀₇-HLA-DRB1*0301/DRA, CYP2D6₃₁₃₋₃₃₂-HLA-DRB1*0301/DRA, CYP2D6₃₉₃₋₄₁₂-HLA-DRB1*0301/DRA, CYP2D6₁₉₉₋₂₁₃-HLA-DRB1*0401/DRA, CYP2D6₄₅₀₋₄₆₄-HLA-DRB1*0401/DRA, CYP2D6₃₀₁₋₃₁₅-HLA-DRB1*0401/DRA, CYP2D6₄₅₂₋₄₆₆-HLA-DRB1*0701/DRA, CYP2D6₅₉₋₇₃-HLA-DRB1*0701/DRA, CYP2D6₁₃₀₋₁₄₄-HLA-DRB1*0701/DRA, CYP2D6₁₉₃₋₂₁₂-HLA-DRB1*0701/DRA, CYP2D6₃₀₅₋₃₂₄-HLA-DRB1*0701/DRA, CYP2D6₁₃₁₋₁₄₅-HLA-DRB3*0202/DRA, CYP2D6₂₁₆₋₂₃₀-HLA-DRB3*0202/DRA, CYP2D6₂₃₈₋₂₅₂-HLA-DRB3*0202/DRA, CYP2D6₁₉₉₋₂₁₃-HLA-DRB4*0101/DRA, CYP2D6₂₃₅₋₂₅₂-HLA-DRB4*0101/DRA, CYP2D6₂₉₃₋₃₀₇-HLA-DRB4*0101/DRA, CYP2D6₂₃₈₋₂₅₂-HLA-DRB5*0101/DRA, CYP2D6₃₈₁₋₃₉₅-HLA-DRB5*0101/DRA, CYP2D6₄₂₉₋₄₄₃-HLA-DRB5*0101/DRA, SLA₃₃₄₋₃₄₈-HLA-DRB1*0301/DRA, SLA₁₉₆₋₂₁₀-HLA-DRB1*0301/DRA, SLA₁₁₅₋₁₂₉-HLA-DRB1*0301/DRA, SLA₃₇₃₋₃₈₆-HLA-DRB1*0301/DRA, SLA₁₈₆₋₁₉₇-HLA-DRB1*0301/DRA, SLA₃₁₇₋₃₃₁-HLA-DRB1*0401/DRA, SLA₁₇₁₋₁₈₅-HLA-DRB1*0401/DRA, SLA₄₁₇₋₄₃₁-HLA-DRB1*0401/DRA, SLA₃₅₉₋₃₇₃-HLA-DRB1*0701/DRA, SLA₂₁₅₋₂₂₉-HLA-DRB1*0701/DRA, SLA₁₁₁₋₁₂₅-HLA-DRB1*0701/DRA, SLA₁₁₀₋₁₂₄-HLA-DRB3*0202/DRA, SLA₂₉₉₋₃₁₃-HLA-DRB3*0202/DRA, SLA₃₄₂₋₃₅₆-HLA-DRB3*0202/DRA, SLA₄₉₋₆₃-HLA-DRB4*0101/DRA, SLA₁₁₉₋₁₃₃-HLA-DRB4*0101/DRA, SLA₂₆₀₋₂₇₄-HLA-DRB4*0101/DRA, SLA₂₆₋₄₀-HLA-DRB5*0101/DRA, SLA₈₆₋₁₀₀-HLA-DRB5*0101/DRA, or SLA₃₃₁₋₃₄₅-HLA-DRB5*0101/DRA;
l) uveitis and the pMHC complex is selected from the group of: arrestin₁₉₉₋₂₁₃-HLA-DRB3*0101/DRA, arrestin₇₇₋₉₁-HLA-DRB3*0101/DRA, arrestin₂₅₀₋₂₆₄-HLA-DRB3*0101/DRA, arrestin₁₇₂₋₁₈₆-HLA-DRB4*0101/DRA, arrestin₃₅₄₋₃₆₈-HLA-DRB4*0101/DRA, arrestin₂₃₉₋₂₅₃-HLA-DRB4*0101/DRA, arrestin₁₀₂₋₁₁₆-HLA-DRB5*0101/DRA, arrestin₅₉₋₇₃-HLA-DRB5*0101, arrestin₂₈₀₋₂₉₄-HLA-DRB5*0101, arrestin₂₉₁₋₃₀₆-HLA-DRB1*0301/DRA, arrestin₁₉₅₋₂₀₉-HLA-DRB3*0202/DRA, arrestin₁₉₉₋₂₁₃-HLA-DRB3*0202/DRA, or arrestin₂₀₀₋₂₁₄-HLA-DRB3*0202/DRA;
m) Sjogren Syndrome and the pMHC complex is selected from the group of: RO60₁₂₇₋₁₄₁-HLA-DRB1*0301/DRA, RO60₅₂₃₋₅₃₇-HLA-DRB1*0301/DRA, RO60₂₄₃₋₂₅₇-HLA-DRB1*0301/DRA, RO60₄₈₄₋₄₉₈-HLA-DRB3*0101/DRA, RO60₃₄₇₋₃₆₁-HLA-DRB3*0101/DRA, RO60₃₆₉₋₃₈₃-HLA-DRB3*0101/DRA, RO60₄₂₆₋₄₄₀-HLA-DRB4*0101/DRA, RO60₂₆₇₋₂₈₁-HLA-DRB4*0101/DRA, RO60₁₇₈₋₁₉₂-HLA-DRB4*0101/DRA, RO60₃₅₈₋₃₇₂-HLA-DRB5*0101/DRA, RO60₂₂₁₋₂₃₅-HLA-DRB5*0101/DRA, RO60₃₁₈₋₃₃₂-HLA-DRB5*0101/DRA, RO60₅₁₋₆₅-HLA-DRB3*0202/DRA, RO60₃₁₂₋₃₂₆-HLA-DRB3*0202/DRA, RO60₃₄₇₋₃₆₁-HLA-DRB3*0202/DRA, LA₂₄₁₋₂₅₅-HLA-DRB1*0301/DRA, LA₁₀₁₋₁₁₅-HLA-DRB1*0301/DRA, LA₁₅₃₋₁₆₇-HLA-DRB1*0301/DRA, LA₁₇₈₋₁₉₂-HLA-DRB3*0101/DRA, LA₁₉₋₃₃-HLA-DRB3*0101/DRA, LA₃₇₋₅₁-HLA-DRB3*0101/DRA, LA₁₃₃₋₁₄₇-HLA-DRB4*0101/DRA, LA₅₀₋₆₄-HLA-DRB4*0101/DRA, LA₃₂₋₄₆-HLA-DRB4*0101/DRA, LA₁₅₃₋₁₆₇-HLA-DRB5*0101/DRA, LA₈₃₋₉₇-HLA-DRB5*0101/DRA, LA₁₃₆₋₁₅₀-HLA-DRB5*0101/DRA, LA₅₀₋₆₄-HLA-DRB3*0202/DRA, LA₈₆₋₁₀₀-HLA-DRB3*0202/DRA, or LA₁₅₄₋₁₆₈-HLA-DRB3*0202/DRA;
n) scleroderma and the pMHC complex is selected from the group of: TOP1₃₄₆₋₃₆₀-HLA-DRB3*0101/DRA, TOP1₄₂₀₋₄₃₄-HLA-DRB3*0101/DRA, TOP1₇₅₀₋₇₆₄-HLA-DRB3*0101/DRA, TOP1₄₁₉₋₄₃₃-HLA-DRB4*0101/DRA, TOP1₅₉₁₋₆₀₅-HLA-DRB4*0101/DRA, TOP1₆₉₅₋₇₀₉-HLA-DRB4*0101/DRA, TOP1₃₀₅₋₃₁₉-HLA-DRB5*0101/DRA, TOP1₃₄₆₋₃₆₀-HLA-DRB5*0101/DRA, TOP1₄₁₉₋₄₃₃-HLA-DRB5*0101/DRA, TOP1₄₂₀₋₄₃₄-HLA-DRB3*0202/DRA, TOP1₄₂₅₋₄₃₉-HLA-DRB3*0202/DRA, TOP1₆₁₄₋₆₂₈-HLA-DRB3*0202/DRA, CENP-C₂₉₇₋₃₁₁-HLA-DRB3*0101/DRA, CENP-C₈₅₇₋₈₇₁-HLA-DRB3*0101, CENP-C₈₈₇₋₉₀₁-HLA-DRB3*0101, CENP-C₂₁₂₋₂₂₆-HLA-DRB4*0101/DRA, CENP-C₆₄₃₋₆₅₇-HLA-DRB4*0101/DRA, CENP-C₈₃₂₋₅₄₆-HLA-DRB4*0101/DRA, CENP-C₁₆₇₋₁₈₁-HLA-DRB5*0101/DRA, CENP-C₂₄₆₋₂₆₀-HLA-DRB5*0101/DRA, CENP-C₈₄₆₋₈₆₀-HLA-DRB5*0101/DRA, CENP-C₁₄₉₋₁₆₃-HLA-DRB3*0202/DRA, CENP-C₈₃₃₋₈₄₇-HLA-DRB3*0202/DRA, or CENP-C₈₄₇₋₈₆₁-HLA-DRB3*0202/DRA;
o) anti-phospholipid syndrome and the pMHC complex is selected from the group of: APOH₂₃₅₋₂₄₉-HLA-DRB3*0101/DRA, APOH₃₀₆₋₃₂₀-HLA-DRB3*0101/DRA, APOH₂₃₇₋₂₅₁-HLA-DRB3*0101/DRA, APOH₂₉₅₋₃₀₉-HLA-DRB3*0101/DRA, APOH₂₈₋₄₂-HLA-DRB4*0101/DRA, APOH₁₇₃₋₁₈₇-HLA-DRB4*0101/DRA, APOH₂₆₄₋₂₇₈-HLA-DRB4*0101/DRA, APOH₂₉₅₋₃₀₉-HLA-DRB4*0101/DRA, APOH₄₉₋₆₃-HLA-DRB5*0101/DRA, APOH₂₆₉₋₂₈₃-HLA-DRB5*0101/DRA, APOH₂₉₅₋₃₀₉-HLA-DRB5*0101/DRA, APOH₃₂₁₋₃₅₅-HLA-DRB3*0202/DRA, APOH₃₂₂₋₃₃₆-HLA-DRB3*0202/DRA, or APOH₃₂₄₋₃₃₈-HLA-DRB3*0202/DRA;
p) ANCA-associated vasculitis and the pMHC complex is selected from the group of: MPO₅₀₆₋₅₂₀-HLA-DRB3*0101/DRA, MPO₃₀₂₋₃₁₆-HLA-DRB3*0101/DRA, MPO₇₋₂₁-HLA-DRB3*0101/DRA, MPO₆₈₉₋₇₀₃-HLA-DRB4*0101/DRA, MPO₂₄₈₋₂₆₂-HLA-DRB4*0101/DRA, MPO₄₄₄₋₄₅₈-HLA-DRB4*0101/DRA, MPO₅₁₃₋₅₂₇-HLA-DRB5*0101/DRA, MPO₉₇₋₁₁₁-HLA-DRB5*0101/DRA, MPO₆₁₆₋₆₃₀-HLA-DRB5*0101/DRA, MPO₄₆₂₋₄₇₆-HLA-DRB3*0202/DRA, MPO₆₁₇₋₆₃₁-HLA-DRB3*0202/DRA, MPO₇₁₄₋₇₂₈-HLA-DRB3*0202/DRA, PRTN3₄₄₋₅₈-HLA-DRB3*0101/DRA, PRTN3₂₃₄₋₂₄₈-HLA-DRB3*0101/DRA, PRTN3₅₉₋₇₃-HLA DRB3*0101/DRA, PRTN3₅₉₋₇₃-HLA-DRB5*0101/DRA, PRTN3₁₁₇₋₁₃₁-HLA-DRB4*0101/DRA, PRTN3₁₆₄₋₁₇₈-HLA-DRB4*0101/DRA, PRTN3₇₁₋₈₅-HLA-DRB4*0101/DRA, PRTN3₂₄₁₋₂₅₅-HLA-DRB5*0101/DRA, PRTN3₁₈₃₋₁₉₇-HLA-DRB5*0101/DRA, PRTN3₆₂₋₇₆-HLA-DRB3*0202/DRA, PRTN3₁₁₈₋₁₃₂-HLA-DRB3*0202/DRA, or PRTN3₂₃₉₋₂₅₃-HLA-DRB3*0202/DRA; or
q) Stiff Man Syndrome and the pMHC complex is selected from the group of: GAD₂₁₂₋₂₂₆-HLA-DRB1*0801/DRA, GAD₅₅₅₋₅₆₉-HLA-DRB1*0801/DRA, or GAD₂₉₇₋₃₁₁-HLA-DRB1*0301/DRA.

In certain aspects, provided herein are methods to treat type I diabetes in a subject in need thereof comprising administering an effective amount of the complex or composition disclosed herein, wherein the complex and the composition may comprise one or more nanoparticle core coupled to a plurality of pMHC complexes, wherein the antigen of the pMHC complex is a diabetes-relevant antigen, wherein the MHC protein of the pMHC complex comprises a MHC class II protein, wherein the nanoparticle core has a diameter of from about 1 nm to about 100 nm, and wherein the pMHC density per nanoparticle core is from about 0.4 pMHC/100 nm² to about 11.6 pMHC/100 nm². In some embodiments, the nanoparticle core has a diameter of from about 1 nm to about 75 nm; from about 1 nm to about 50 nm; from about 1 nm to about 25 nm; from about 1 nm to about 25 nm; from about 5 nm to about 100 nm; from about 5 nm to about 50 nm; or from about 5 nm to about 25 nm, or from about 15 nm to about 25 nm, or about 20 nm. In some embodiments, the nanoparticles core has a diameter of from about 25 nm to about 60 nm, or from about 25 nm to about 50 nm, or from about 20 nm to about 40 nm, or from about 15 nm to about 50 nn, or from about 15 nm to about 40 nm, or from about 15 nm to about 35 nm, or from about 15 nm to about 30 nm, or from about 15 nm to about 25 nm, or alternatively about 15 nm, or about 20 nm, or about 25 nm, or about 30 nm, or about 35 nm, or about 40 nm. In some embodiments, the nanoparticle core has a pMHC density of from about 0.4 pMHC/100 nm² to about 11.6 pMHC/100 nm², or from about 0.4 pMHC/100 nm² to about 11.0 pMHC/100 nm², or from about 0.4 pMHC/100 nm² to about 10 pMHC/100 nm², or from about 0.4 pMHC/100 nm² to about 9 pMHC/100 nm², or from about 0.4 pMHC/100 nm² to about 8 pMHC/100 nm², or from about 0.4 pMHC/100 nm² to about 7 pMHC/100 nm², or from about 0.4 pMHC/100 nm² to about 6 pMHC/100 nm², or from about 0.4 pMHC/100 nm² to about 5 pMHC/100 nm², or from about 0.4 pMHC/100 nm² to about 4 pMHC/100 nm², or from about 0.4 pMHC/100 nm² to about 3 pMHC/100 nm², or from about 0.4 pMHC/100 nm² to about 2 pMHC/100 nm², or from about 0.4 pMHC/100 nm² to about 1.5 pMHC/100 nm². In some embodiments, the nanoparticle core has a pMHC density of from about 0.4 pMHC/100 nm² to about 6 pMHC/100 nm² or from about 0.4 pMHC/100 nm² to about 1.5 pMHC/100 nm². In some embodiments, the pMHC complex comprises an antigen derived from one or more of IGRP or PPI. In some embodiments, the pMHC complex comprises an antigen selected from one or more of the group: IGRP₁₃₋₂₅, PPI₇₆₋₉₀, or PPI_{76-90(K88S)}. In some embodiments, the pMHC complex comprises HLA-DR. In some embodiments, the pMHC complex comprises HLA-DR/DRA. In some embodiments, the pMHC complex comprises, or alternatively consists of, or further yet consists essentially of one or more of IGRP₁₃₋₂₅-HLA-DRB1*0301/DRA, PPI₇₆₋₉₀-HLA-DRB1*0401/DRA, or PPI_{76-90(K88S)}-HLA-DRB1*0401/DRA.

Methods to determine and monitor the therapy are known in the art and are briefly described herein. When delivered in vitro, administration is by contacting the composition with the tissue or cell by any appropriate method, e.g., by administration to cell or tissue culture medium and is useful as a screen to determine if the therapy is appropriate for an individual or to screen for alternative therapies to be used as a substitute or in combination with the disclosed compositions. When administered in vivo, administration is by systemic or local administration. In vivo, the methods can be practiced on a non-human animal to screen alternative therapies to be used as a substitute or in combination with the disclosed compositions prior to human administration. In a human or non-human mammal, they are also useful to treat the disease or disorder.

The above methods require administration of an effective amount of an antigen/MHC complex operatively coupled to a nanoparticle as disclosed herein above, which may optionally further comprise, alternatively consist essentially of, or yet further consist of co-stimulatory molecules and/or cytokines coupled to the same nanoparticle. Disease targets and relevant antigens are disclosed herein above.

Details regarding modes of administration in vitro and in vivo are described herein above.

This disclosure also provides use of the NP-complexes for the preparation of medicaments for the treatment and/or prevention of diseases and disorders as described herein.

### Monitoring Therapy and Detection of T Cells

Some aspects of the present disclosure relate to methods of detecting and/or monitoring a population of immune cells, preferably T cells comprising administering a labeled antigen-MHC complex where a subject has received an pMHC-NP or composition as disclosed herein.

In certain aspects, provided herein are methods to detect a population of T_{R}1 cells and/or effector T cells in an antigen specific manner in a subject that has received the complex or the composition disclosed herein. The method comprises, alternatively consists of, or yet further consists essentially of, contacting a sample suspected of comprising the T_{R}1 cells with an effective amount of labeled pMHC complex to form a multimer complex, and detecting any multimer complex, thereby detecting the population of T_{R}1 cells. In some embodiments, the method further comprises, alternatively further consists of, or yet further consists essentially of staining any T cell population using a labeled multimer complex. In some embodiments, the step of detecting the population of T_{R}1 cells comprises flow cytometry to detect any multimer complex. In some embodiments, the method further comprises, or alternatively consists of, or yet further consists essentially of administering the complex or composition to the subject.

In certain aspects, provided herein are methods to detect a population of T_{R}1 cells and/or effector T cells in an antigen specific manner in a subject that has received the complex or the composition disclosed herein The method comprises, alternatively consists of, or yet further consists essentially of any one of the following assays: cytokine ELISPOT assay, a multimer-guided epitope analysis, or a multimer-pull-down assay. In some embodiments, the method further comprises, alternatively further consists of, or yet further consists essentially of administering the complex or the composition disclosed herein.

In other aspects, provided herein are methods to monitor the expansion of a population of antigen-specific T_{R}1 and/or effector T cells in a subject. The method comprises, alternatively consists of, or yet further consists essentially of: a) administering to a subject an effective amount of the complex or the composition disclosed herein, wherein the disease-relevant antigen of the pMHC complex is selected to expand the antigen-specific T_{R}1 and/or effector T cells; b) isolating a suitable sample from the subject suspected of containing the population; c) contacting the sample with an effective amount of labeled pMHC complex to form a multimer complex, and detecting any multimer complex; and d) quantifying the number of antigen-specific T_{R}1 and/or effector T cells in the population. In some embodiments, the method further comprises, alternatively further consists of, or yet further consists essentially of staining any multimer complex. In some embodiments, the step of quantifying the number of antigen-specific T_{R}1 and/or effector T cells comprises flow cytometry and/or ELISA. In some embodiments, the method further comprises, alternatively further consists of, or yet further consists essentially of administering the complex or the composition disclosed herein.

There are many types of immunoassays that can be implemented. Immunoassays encompassed by the present disclosure include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

One method for quantifying the number of circulating antigen-specific immune cells is the tetramer assay. In this assay, a specific epitope is bound to synthetic multimeric forms of fluorescently labeled MHC molecules. Since immune cells recognize antigens in the form of short peptides bound to MHC molecules, cells with the appropriate T cell receptor will bind to the labeled tetramers and can be quantified by flow cytometry. Although this method is less time-consuming than an ELISPOT assay, the multimer assay measures only binding, not function. Not all cells that bind a particular antigen necessarily become activated. However, correlation between ELISPOT, multimer, and cytotoxicity assays has been demonstrated.

Immunoassays generally are binding assays. Certain immunoassays, including the various types of enzyme linked immunosorbent assays (ELISAs), radioimmunoassays (RIA) or bead based assays, such as Luminex® technology, are known in the art. Immunohistochemical detection using tissue sections is also particularly useful.

In one example of ELISA, the antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label. Variations on ELISA techniques are known to those of skill in the art.

Competition ELISAs are also possible in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal.

Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

In ELISAs, it is more customary to use a secondary or tertiary detection means rather than a direct procedure. Thus, after binding of the antigen or antibody to the well, coating with a non reactive material to reduce background, and washing to remove unbound material, the immobilizing surface is contacted with the clinical or biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

Additionally, flow cytometry may be used to detect and quantitate particular cell subtypes according to cell surface markers. Common means of detection and quantitation via flow cytometry include the use of fluorescent labeled beads that bind to cell surface markers specific to each immune cell subtype, e.g. CD 4 specific beads, to select for CD 4+ T cells, etc.

### Kits

Also provided herein are kits comprising the nanoparticle complex as described herein or the compositions as described herein for diagnostic, prognostic or therapeutic use. Additional reagents and/or instructions can further be provided as necessary.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. The present examples, along with the methods described herein are presently representative of embodiments and are exemplary, and are not intended as limitations on the scope of the disclosure. Changes therein and other uses which are encompassed within the spirit of the disclosure as defined by the scope of the claims will occur to those skilled in the art.

### Example 1. Polymeric and Dendrimer Nanoparticle Cores for Autoimmunity and Immunity

The enormous antigenic complexity of autoimmune diseases and other chronic inflammatory phenomena, including allergy, is a barrier to the design of strategies that can purge the immune system of auto- or allergen-reactivity without impairing systemic immunity; current systemic immunosuppressive approaches compromise immunity to infections and cancer.

Thus, in one aspect, the present disclosure establishes that systemic delivery of nanoparticles (NPs) coated with autoimmune or allergic disease-relevant peptide-major histocompatibility complex (pMHC) class II molecules triggers the expansion of cognate T-regulatory type 1 (T_{R}1) CD4+ T-cells in vivo in different disease models and genetic backgrounds, leading to resolution of various autoimmune or allergic phenomena, including spontaneous type 1 diabetes, experimental autoimmune encephalomyelitis and house dust mite-induced asthma. These nanomedicines promote the differentiation of disease-primed autoreactive T-cell precursors into disease-suppressing T_{R}1 cells, which then go on to suppress autoreactive and allergen-specific T-cell responses in the affected tissues by targeting autoantigen- or allergen-loaded antigen-presenting cells (APCs), while sparing non-loaded APCs elsewhere. Suppression of disease does not impair the host's ability to clear viral infections or to mount antibody responses to conventional vaccines; is mediated by local secretion of IL-10 and TGF-beta in response to these cognate T_{R}1-APC interactions; and involves a profound inhibition of the ability of local (but not distal) APCs to secrete pro-inflammatory cytokines and activate other T-cells. Furthermore, it is found that the expanded T_{R}1 cells promote the differentiation of cognate B-lymphocytes into IL-10-producing B-regulatory cells in vivo, which contribute to the remarkable therapeutic activity of this therapeutic platform. Importantly, the examples demonstrate that human type 1 diabetes-relevant nanomedicines can expand human T_{R}1 cells in NSG mice engrafted with peripheral blood mononuclear cells from patients, demonstrating the translational potential of this approach. Thus, pMHC class II-based nanomedicines may represent the long-sought-after antigen-specific therapy for autoimmune and allergic inflammation. Similar results can be achieved with pMHC class I-based nanomedicines for the expansion of the appropriate T cell population.

It was determined that the therapeutic properties of these nanomedicines are primarily a function of MHC density (inter-molecular distance). Mathematical modeling of experimental data indicates that, for any given pMHC valency, small but densely coated NPs will have superior biological and therapeutic activity.

In one aspect, superior results are shown for NP core diameter around ˜8-12 nm. The MHC-binding capacity of the pegylated iron oxide NPs lies at ˜55 pMHCs on a 68 nm hydrodynamic diameter NP.

By building MHC-based nanomedicines using third generation poly-L-lysine-based dendrimers (DGLs; 7 nm), this limitation is overcome. The ordered structure of the pMHC-acceptor PEGs on these compounds increases the ligand-binding capacity (hence molecular density) several fold (52 pMHCs on 19 nm hydrodynamic diameter pMHC-DGLN vs. 55 pMHCs on 68 nm diameter pMHC-IONP, resulting in a several fold increase in pMHC density, a critical parameter for biological activity).

Dendrimers are highly branched macromolecules having a tree-like structure with branches growing from a core. They are well known for their three-dimensional, monodispersed, highly branched macromolecular nanoscopic architecture with a number of reactive end groups. These features make dendrimers popular instruments for drug, peptide, and gene delivery in addition to many other biomedical applications.

The widely investigated dendrimers are mainly bear primary amine groups on the branched surface, such as poly (amidoamine) (PAMAM) and poly-L-lysine (PLL) based dendrimers. These dendrimers are soluble in water at the physiological pH due to the presence of charged terminal NH3⁺ groups. However, cationic PAMAM dendrimers exhibit bio-incompatibility, non-degradability and positive-associated cytotoxicity, which limit their wide application in vitro and in vivo.

Cationic PLL are promising new candidates due to their biodegradable properties. A previous study reported that free lysine and larger species (non-dendrimer) appeared in plasma at 1 h postdose of L-lysine capped dendrimers, which indicated the quick degradation of PLL in vivo. (Bailey-Bucktrout, S. L. et al. (2013) Immunity 39:949-962). However, quick degradation is not a benefit for maintaining an effective therapeutic level. Fortunately, it has been reported that fully PEGylated PLL dendrimers had a greater ability to increase plasma stability and circulation time, meanwhile completely masking the positive charge on the surface. PLL-based dendrimers have already been exploited in constructing drug delivery systems. Kaminskas and co-workers conjugated methotrexate (MTX) to a series of PEGylated PLL dendrimers, and demonstrated their potential as long-circulating vectors for the delivery and tumor-targeting of hydrophobic drugs. Others have attached camptothecin (CPT) covalently to PEGylated PLL dendrimers, and demonstrated the significantly prolonged survival in tumor-bearing mice compared to free CPT. However, most of the PLL-based dendrimers used were synthesized by the researchers themselves. The structures of these PLL-based dendrimers are not exactly the same, which significantly limits the prevalence of these dendrimers.

Dendrigraft poly-L-lysines (DGLs), a kind of PLL-based dendrimers, are now commercially available. They are composed of 100% L-lysine, biodegradable, monodispersed, and well-defined, possessing the main properties of PLL-based dendrimers. Current studies are focused on the utility of DGLs for drug or gene delivery. To the best of Applicant's knowledge, DGLs have never been used in the field of presenting pMHC to T cells in blood circulation. In this study, DGLs of generation 3 (G3) (123 amino groups, 7 nm) were used as a scaffold to present pMHC and to evaluate the immunology activity.

Preparation, Purification and Characterization of pMHC-PEG-DGL

In this study, G3 of DGLs with 123 amino groups is selected as the vector material. Its surface is coated with heterobifunctional crosslinker, NHS-PEG₄-Azido (MW 388 g/mol) through the specific reaction between primary amino groups and activated NHS ester. The heterobifunctional PEG, maleimide-PEG-alkyne (Mal-PEG-Alkyne, MW 2,000) can conjugate with pMHC molecule via thiol-maleimide reaction. The free alkyne on the end of pMHC-PEG conjugates could react with azido coated DGLs through Click chemistry. The resulting NPs were purified by gel filtration to remove the unconjugated pMHC. The significant charge changes of DGLs before and after coating can be monitored by Z-potential and agarose gel electrophoresis. The resulting NPs can be characterized by DLS, Z-potential, SDS-page and TEM.

### Dendri-Graft Poly-L-Lysine Generation 3 (DGLs G3)

In another study, dendri-Graft Poly-L-Lysines Generation 3 (DGLs G3) was purchased from COLCOM in France. DGLs G3 is a synthetic polymer with a structure constituted by nine equivalent dendrons linked to a core. The core is a linear poly-L-lysine with an average of eight monomers. Each dendron looks like the traditional Tam-type dendrons synthesized from Merrifield resins. DGLs G3 is a non-immunogenic carrier with a molecular weight of 22 KDa and 123 terminal primary amino groups (—NH₂) for functionalization and conjugation (FIG. 3).

### Synthesis of Dendri-Graft Poly-L-Lysines-Azido (—N₃) (“DGLN”)

DGLs were first functionalized with N-Hydroxysuccinimide-PEG₄-Azido (NHS-PEG₄-Azido, MW 388.37, purchased from Conju-Probe, Canada) to: 1) enable the conjugation of pMHC; and 2) neutralize the positive surface charge of non-functionalized DGLs.

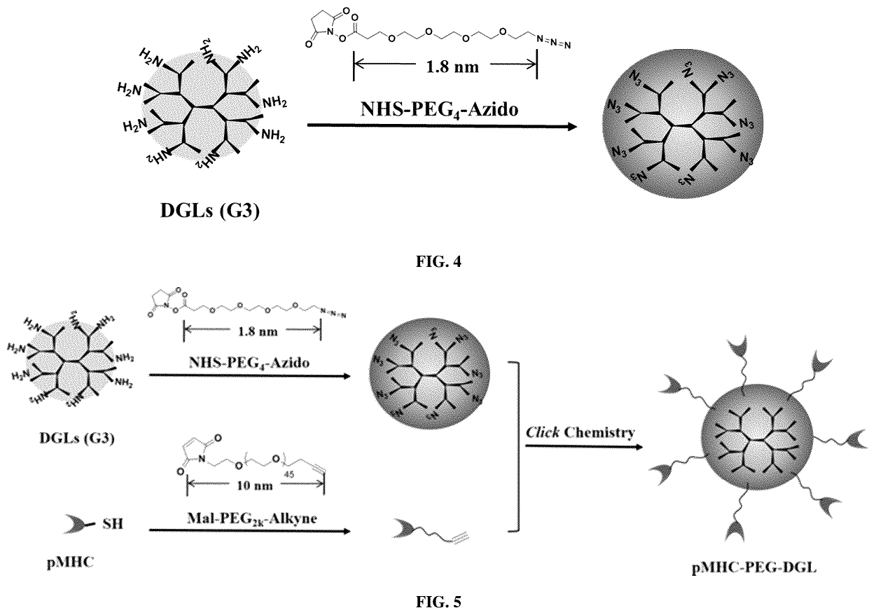

The DGLs surface functionalization was achieved by using a hetero-bifunctional crosslinker, NHS-PEG₄-Azido. Activated NHS ester easily reacts with primary amino groups on DGLs in a mild aqueous environment. About 1 mg of DGL-NH₂ was dissolved in PBS, at a pH of about 8.0. About 4.3 mg of NHS-PEG₄-N₃ (—NH₂:PEG₄=1:2, mol:mol) was added into the solution and reacted at room temperature for about 2 hours. After reaction, the DGL-N₃ was washed by ultrafiltration (MW cutoff 3000) with PBS at about pH 7.4 three times to remove unreacted NHS-PEG₄-N₃ (FIG. 4).

pMHC Conjugation to DGLN (FIG. 5)

To conjugate pMHC monomers to the surface of DGLN, a single-chain NRP-V7/K^{d} construct engineered to encode a carboxyterminal Cys (—SH) is first pegylated and produced in CHO cells (referred to as V7CHO-Cys). Briefly, a 3.5 mL solution of V7CHO-Cys (3.58 mg/mL) in PBS pH 7.4 was mixed with 24 μL of 500 mM EDTA, 375 uL of 1 M NaCl, 500 μL of 200 mM PB buffer and 1.625 mL ETF water. 4 mg of Malimide-PEG₂ₖ-Alkyne was then added to the mixture (final reaction volume was 6.0 mL) and allowed to react overnight at R.T. The reaction solution was then dialyzed against PBS pH 7.4 at 4° C. for 48 h.

V7CHO-PEG₂ₖ-Alkyne solution was next concentrated to a final volume of 3.5 mL in PBS pH 7.4 in a nitrogen atmosphere and added 60 μL of DGLN (5 mg/mL in PBS), 150 μL ascorbic acid (50 mM in PBS) and 175 μL Cu-TBTA, which were allowed to react for 24 h at R.T. After reaction, the nanoparticles were purified via ultrafiltration (MW cutoff 100 KDa) against PBS pH 7.4, 6 times.

### Biochemical and Biophysical Analyses of the Conjugates

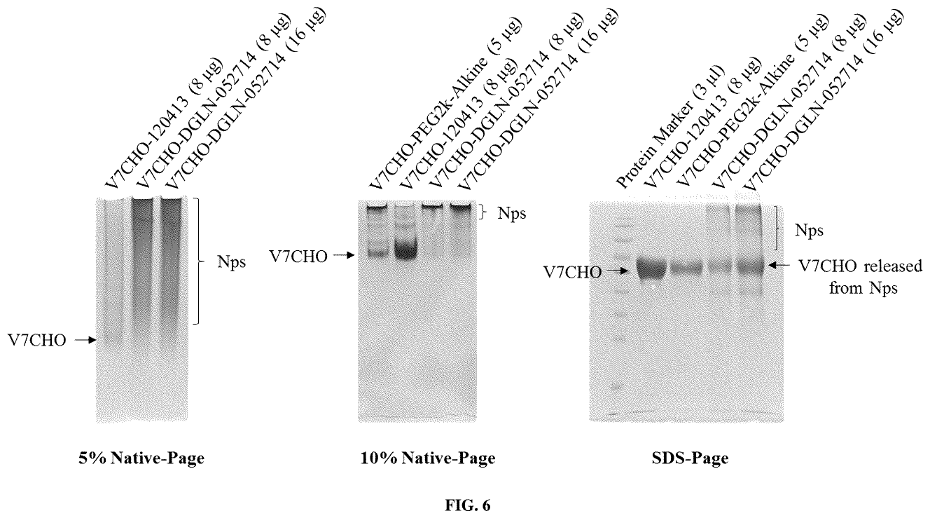

The conjugates were analyzed via native and denaturing (SDS) PAGE. FIG. 6 shows the presence of an obvious Coomassie-blue stained smear under native-PAGE conditions (left two panels), detecting pMHC-conjugated DLGN, in the absence of free (unconjugated) V7CHO monomer. Electrophoresis of these compounds under denaturing/reducing conditions revealed the release of V7CHO from the NPs, confirming that pMHC conjugation to DLGN was successful.

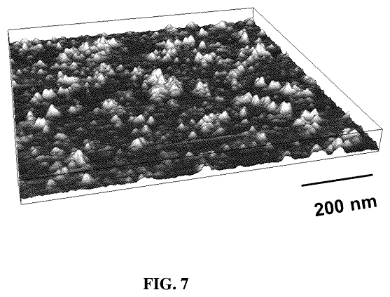

The biophysical properties of the pMHC-DGLN compound were next ascertained using atomic force microscopy (AFM) (FIG. 7). Briefly, the NP solution was layered on mica and observed under an AFM. V7CHO-DGLN displayed a spherical conformation with an average diameter of 19.95±0.25 nm (AFM measurements). The NPs were distributed well with monodisperity. The polydispersity Index (PI) and the hydrodynamic diameter in aqueous solution was tested by DLS. The concentration used for the AFM sample preparation was 4 μg DGLN/mL (equal to 4.38×10¹³ NPs/mL). Analysis was done using 5 μL of this solution (˜2.19×10¹¹ NPs) on a 1 um×1 um scanning area.

Bradford analysis indicated that the pMHC content of the compound described above corresponded to 52 pMHC monomers on each NP.

Lastly, to ascertain if this compound had agonistic activity on cognate T-cells, its ability to trigger the secretion of IFNγ by NRP-V7/K^{d}-specific CD8+ T-cells purified from 8.3-TCR-transgenic NOD mice was measured. Briefly, 8.3-CD8+ T-cells were cultured in the presence of free V7CHO protein, pegylated V7CHO, V7CHO-DGLN or DGLN for 48 h. The IFN-γ content was subsequently measured in the supernatants by ELISA. FIGS. 8A-8B show that V7CHO-DGLN and, to a much lesser extent, pegylated V7CHO, had very high, concentration-dependent agonistic activity on these T-cells, demonstrating the functional properties of these compounds.

Preparation, Purification and Characterization of pMHC-PEG-DSPE Micelles.

Amphiphilic block copolymers assemble into nano-scaled core-shell structures, polymeric micelles, which have been of considerable interest for delivering drugs with poor water solubility. Poly(ethylene glycol)-distearoylphosphatidylethanolamine (PEG-DSPE) block copolymers are safe, biocompatible and have been approved by the Food and Drug Administration for clinical applications. DSPE-PEG has been widely used in the preparation of liposomes, polymeric nanoparticles, polymer hybrid nanoparticles, and solid lipid nanoparticles, among others. The amphiphilic copolymers are nanostructures composed by a hydrophobic core (DSPE) and a hydrophilic shell (PEG). The core-shell structure can encapsulate and carry poorly water-soluble drugs to congregate in the core of DSPE, and the PEG shell reduces the in vivo clearance and the adsorption of plasma proteins. Therefore, utilizing DSPE-PEG for the formation of nanostructures could prolong the body circulation. Most importantly, the critical micelle concentration (CMC) of the DSPE-PEG is extremely low (10⁻⁵ M). This property results in some positive functions of formulated micelles such as greater solubilization of hydrophobic drugs and more thermodynamic stability against dilution with the large volume of the blood following intravenous administration.

To decorate pMHC on the surface of polymeric micelles, DSPE-PEG-maleimide (DSPE-PEG-Mal) were chosen as copolymers. The DSPE-PEG polymeric micelles are prepared by solvent evaporation method as reported in Vakil, R. et al. (2008) Mol Pharm 5: 98-104 and Musacchio, T. et al. (2009) Mol Pharm 6:468-479. In brief, DSPE-PEG-Mal was dissolved in methanol in a round-bottom flask. The organic solvent mixture was evaporated under high vacuum to produce a thin film of copolymers. This film was further dried under vacuum overnight to remove any traces of remaining solvents. Then, the dry polymeric film was dissolved in PBS pH 7.4 to self-assemble into micelles with maleimide groups on the surface. pMHC could be conjugated onto the micellar surfaces through a thiol-maleimide specific reaction. The resulting NPs were purified by gel filtration to remove the unconjugated pMHC. After that, the resulting NPs can be characterized by DLS, Z-potential, SDS-page and TEM.

### Example 2

### Expansion of Disease-Specific T_{R}1 Cells

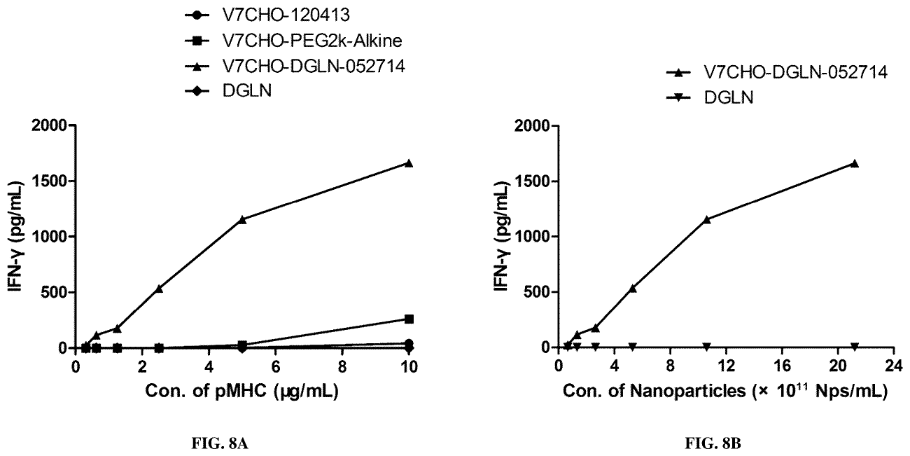

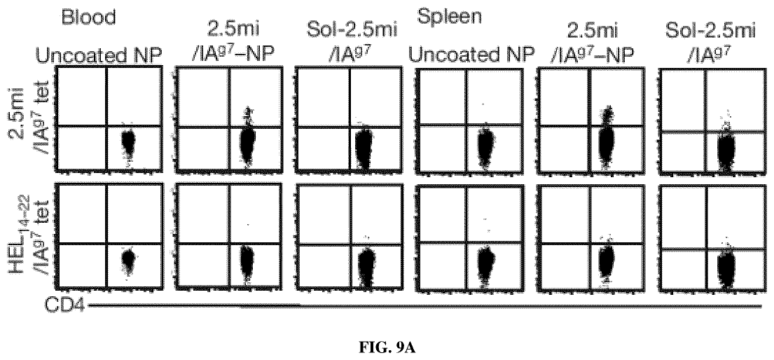

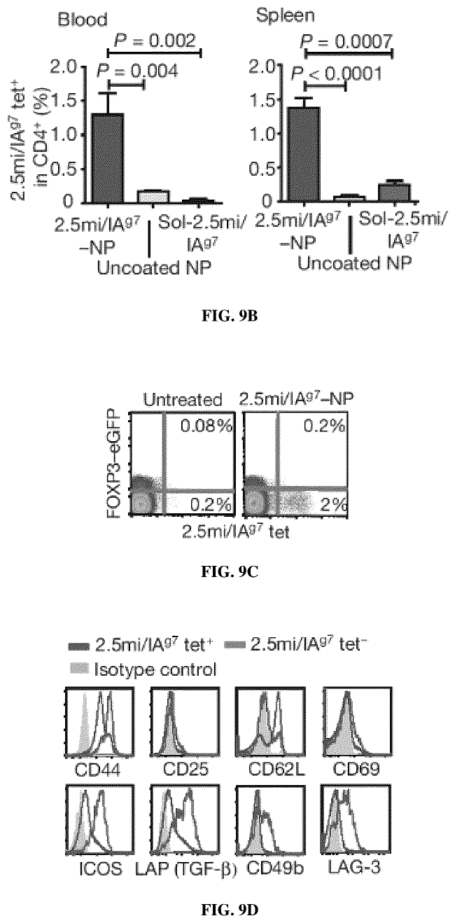

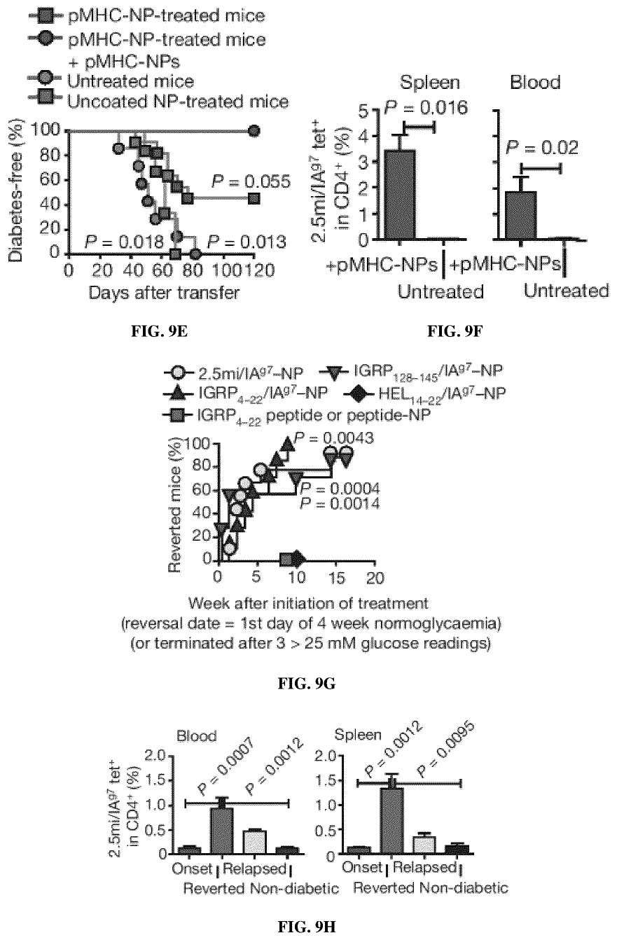

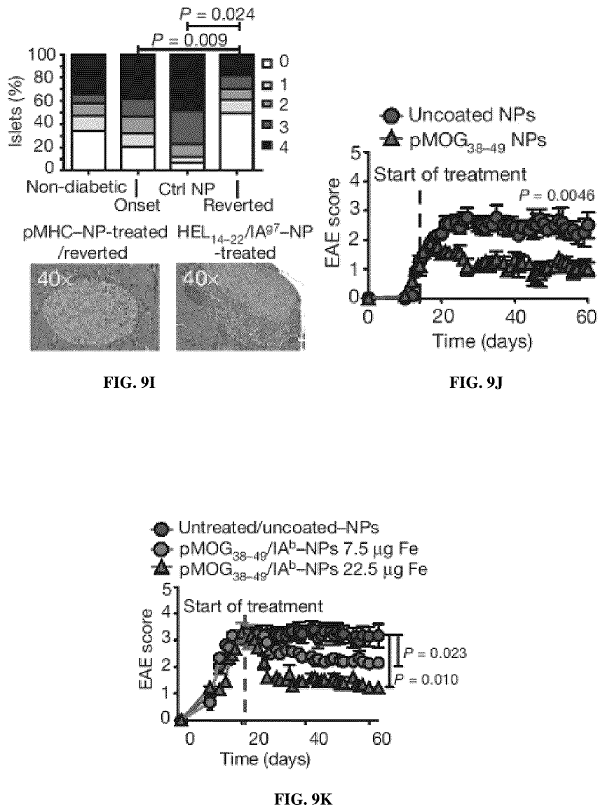

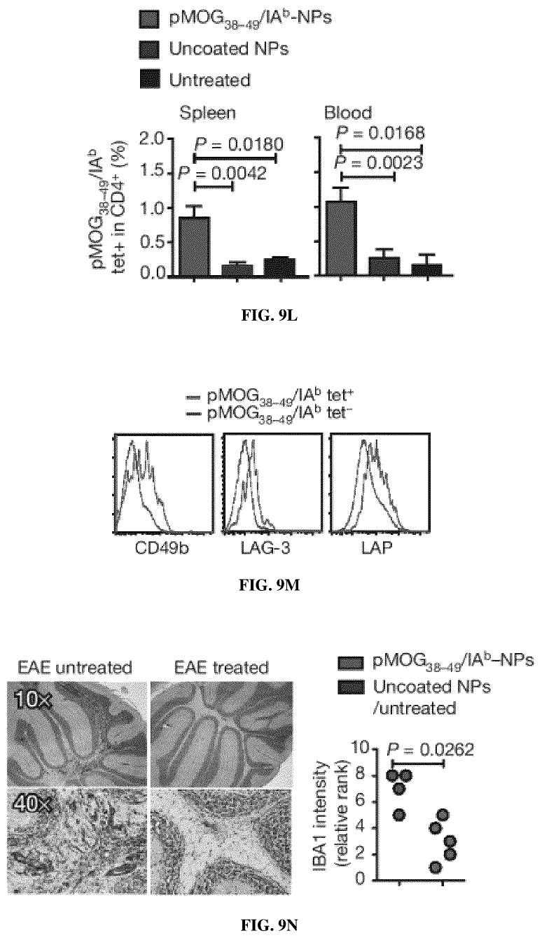

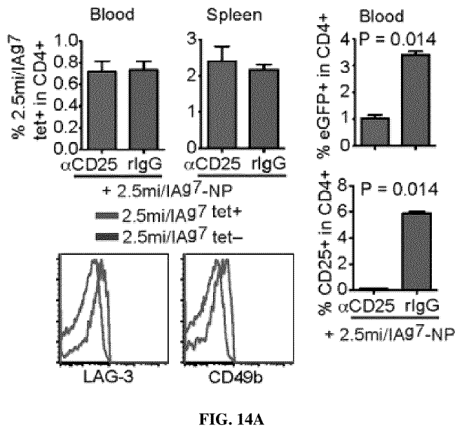

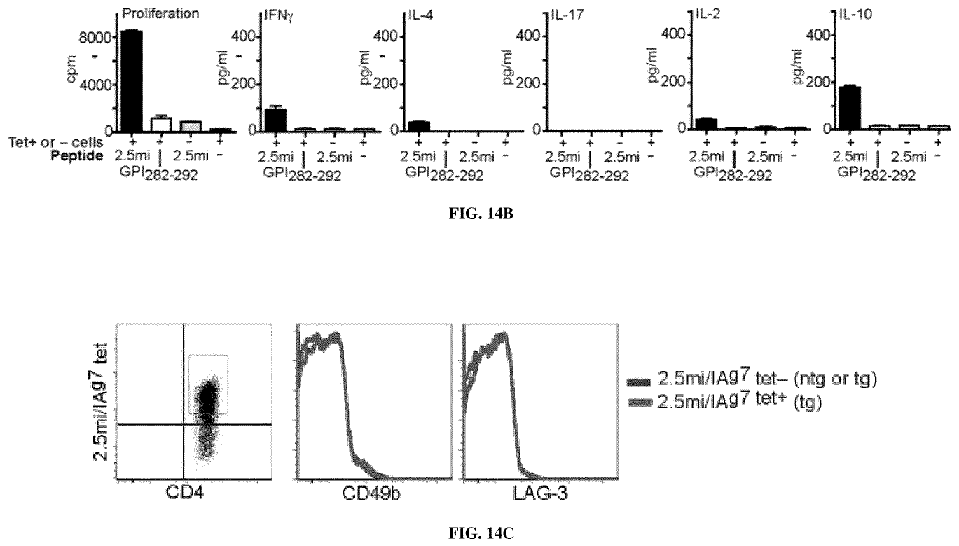

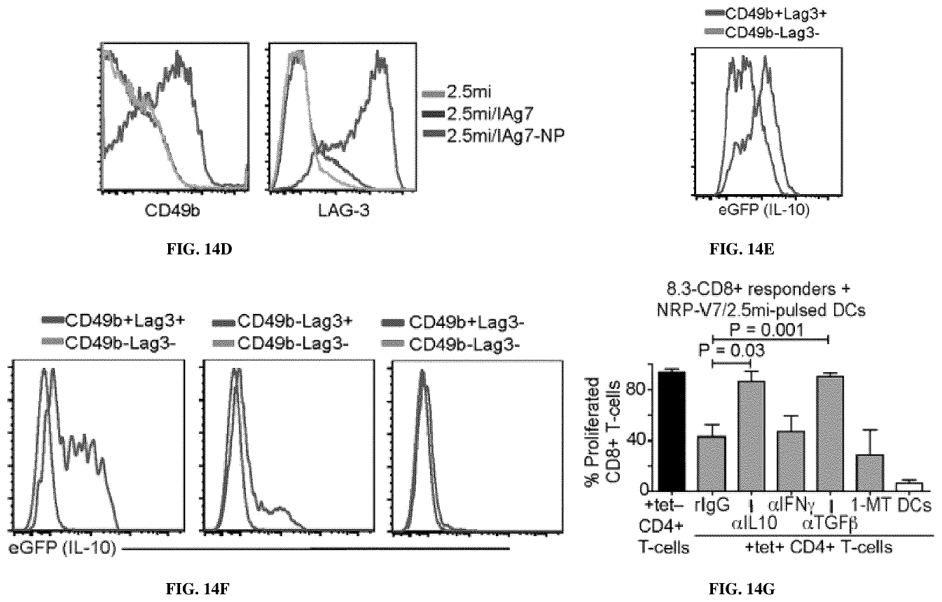

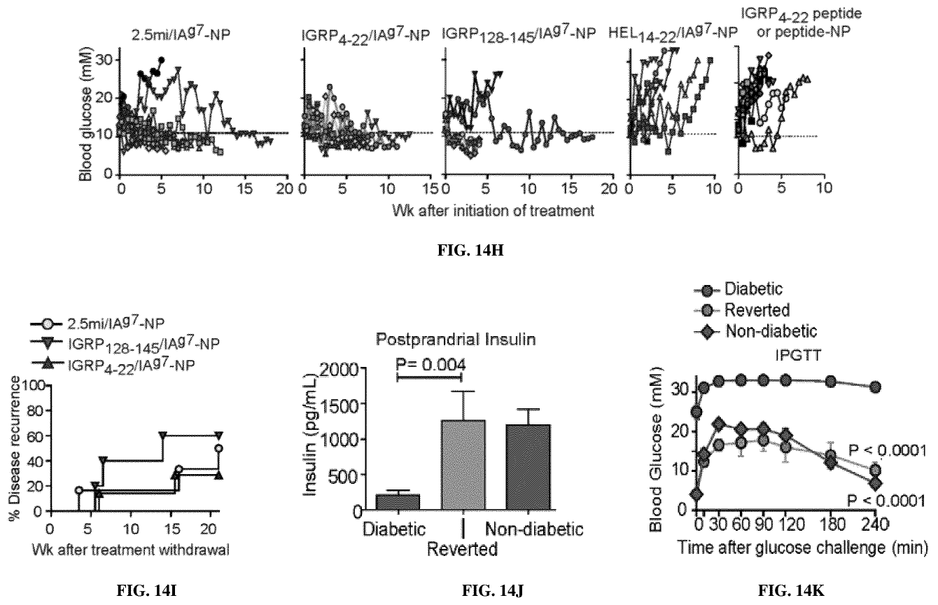

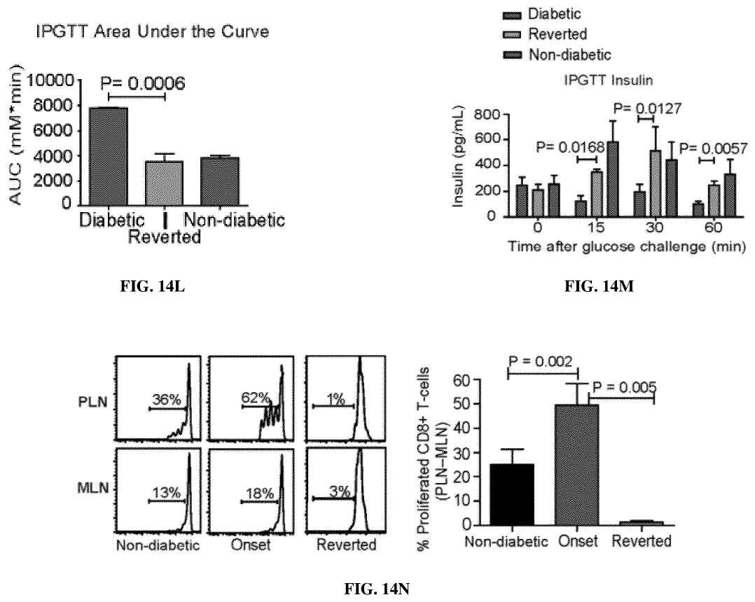

Applicant treated non-obese diabetic (NOD) and NOD Foxp3-eGFP mice expressing enhanced green fluorescent protein (eGFP) under the control of the mouse Foxp3 promoter) with uncoated nanoparticles or nanoparticles coated with a pMHC, 2.5mi/IA^{g7} (Stratmann, T. et al. (2003) J. Clin. Invest. 112:902-914), recognized by the diabetogenic BDC2.5-specific T-cell receptor (TCR), or with 2.5mi/IA^{g7} monomers. Nanoparticles coated in 2.5mi/IA^{g7} induced expansion of cognate CD4⁺ T cells in blood and spleens of all mice (FIGS. 9A, 9B). These cells had a memory-like (CD44^{hi}CD62L^{low}) FOXP3⁻ T_{R}1-like phenotype, expressing ICOS, latent-associated TGF-β and the T_{R}1 markers CD49b and LAG-3 (FIGS. 9C, 9D). A similar outcome was observed in mice treated with 2.5mi/IA^{g7}-NPs upon depletion of CD4⁺CD25⁺ T cells (FIG. 14A). Unlike their tetramer counterparts, these cells proliferated and secreted IL-10 and to a lesser extent IFNγ, but not IL-2, IL-4 or IL-17, in response to dendritic cells (DCs) pulsed with the 2.5mi peptide (FIG. 14B). Real-time reverse-transcription (RT)-PCR analyses confirmed the T_{R}1-like phenotype of these cells (Tables 3A-3B).

To determine if pMHCII-NPs could directly trigger T_{R}1 marker and IL-10 expression on cognate CD4⁺ T cells, Applicant cultured naive and anti-CD3 plus anti-CD28 monoclonal antibody (mAb)-preactivated 2.5mi/IA^{g7}-tetramer⁺CD4⁺ T cells from BDC2.5-TCR-transgenic NOD Foxp3-eGFP or NOD Il10^{GFP} mice (carrying an eGFP insertion in the 1110 locus) (Kamanaka, M. et al. (2006) Immunity 25:941-952) in the presence of 2.5mi/IA^{g7}-NPs, 2.5mi peptide or 2.5mi/IA^{g7} monomer. Naive T cells expressed neither CD49b nor LAG-3, even after incubation with 2.5mi/IA^{g7}-NPs, 2.5mi/IA^{g7} monomer or 2.5mi peptide (FIGS. 14C, 14D). However, preactivated T cells upregulated both markers as well as eGFP (IL-10) only in response to 2.5mi/IA^{g7}-NPs (FIGS. 14D, 14E). In agreement with this, expression of IL-10 in NOD Il10^{GFP} mice treated with 2.5mi/IA^{g7}-NPs was largely restricted to the CD49b+LAG-3⁺CD4+subset (FIG. 14F).

In vitro, the tetramer⁺CD4⁺ T cells of pMHC-NP-treated mice suppressed the proliferation of non-cognate (islet-specific glucose-6-phosphatase catalytic subunit-related protein (IGRP)- or LCMV Gp33-specific) CD8⁺ T cells in response to peptide-pulsed DCs, in an IL-10- and TGF-β-dependent manner (FIG. 14G). In vivo, splenic CD4+ T cells from donors treated with pMHC-NPs suppressed diabetes development in T-cell-reconstituted NOD scid (also known as NOD Prkdc^{scid}) hosts (FIG. 9E), an effect that was potentiated by treating hosts with pMHC-NPs (FIGS. 9E, 9F).

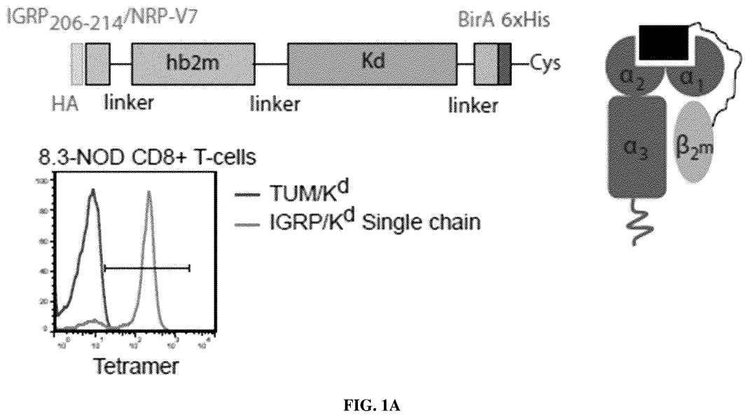

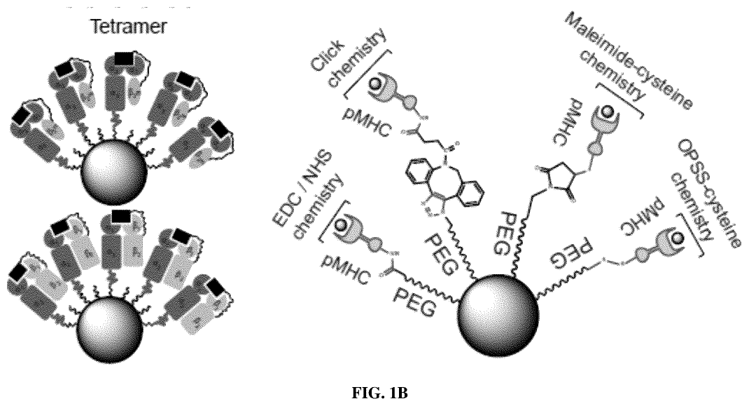

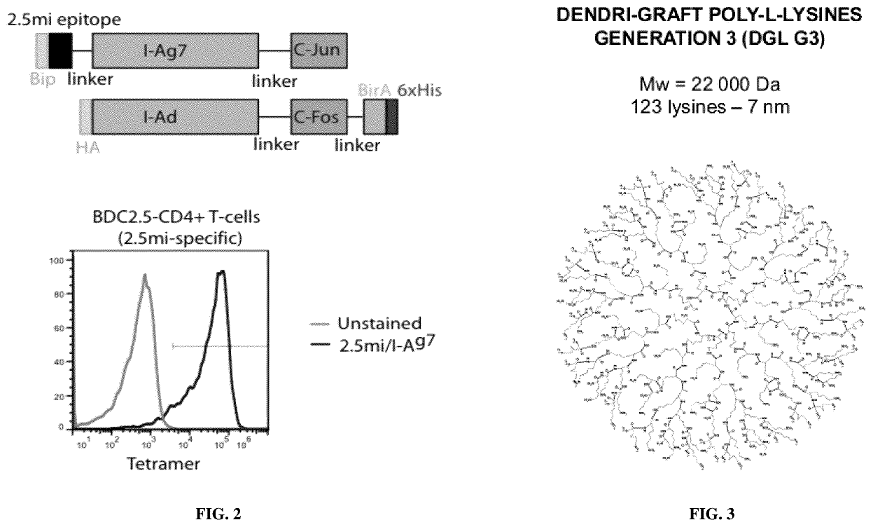

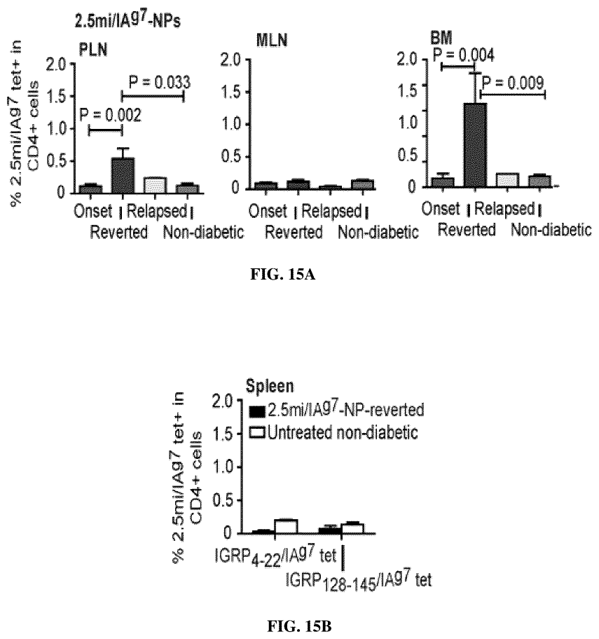

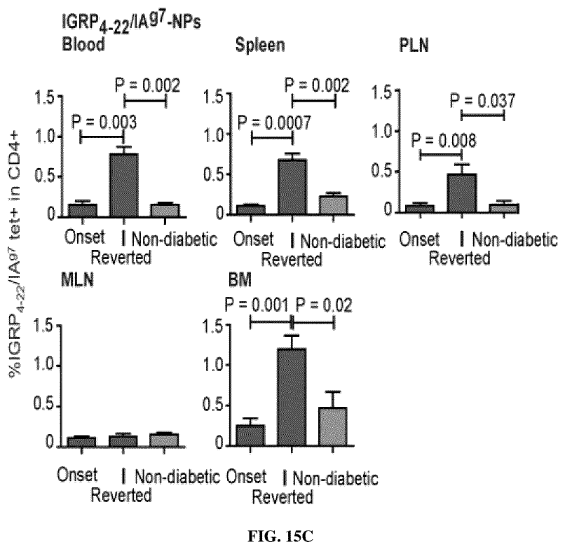

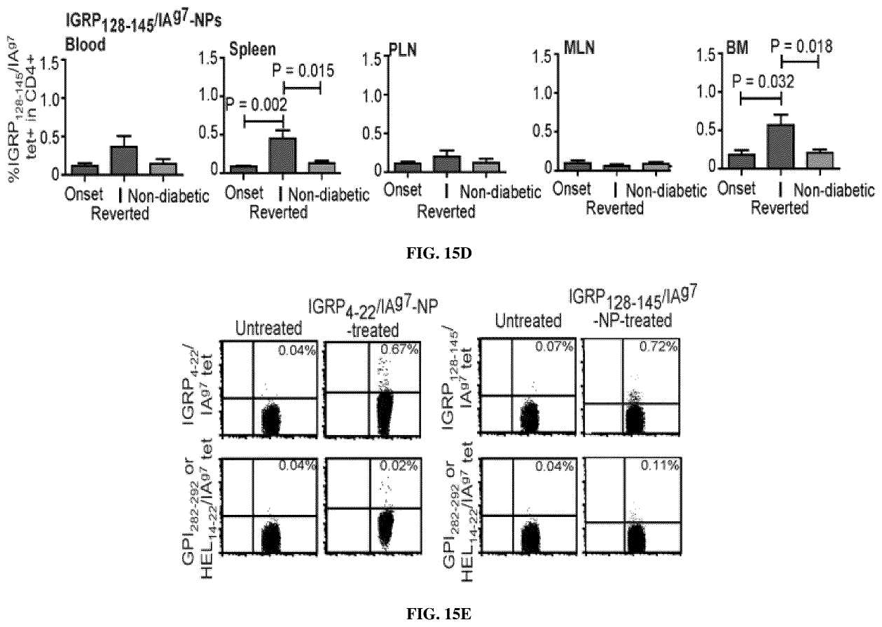

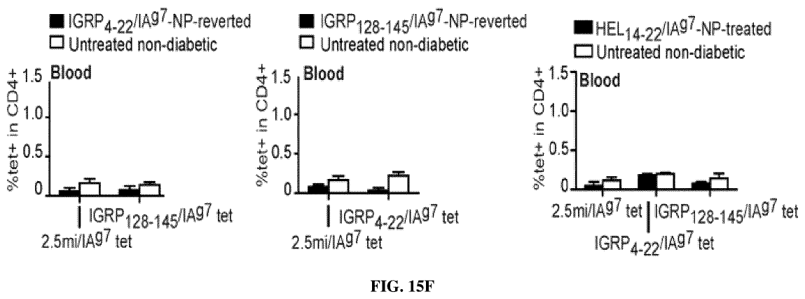

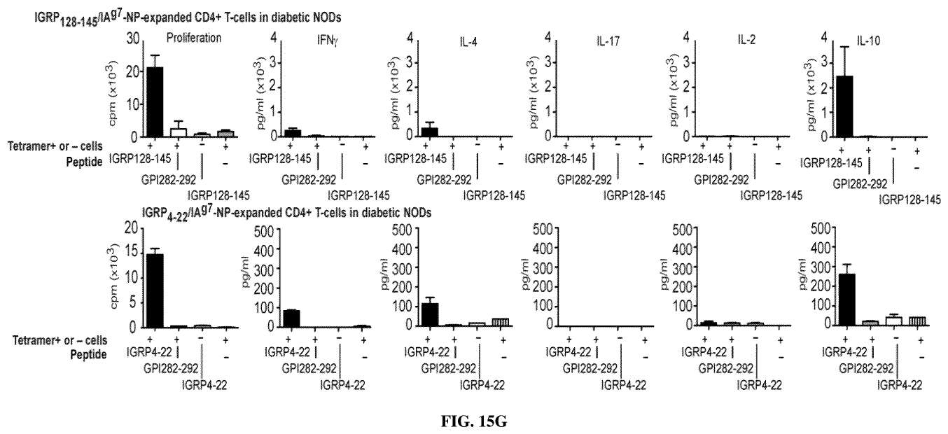

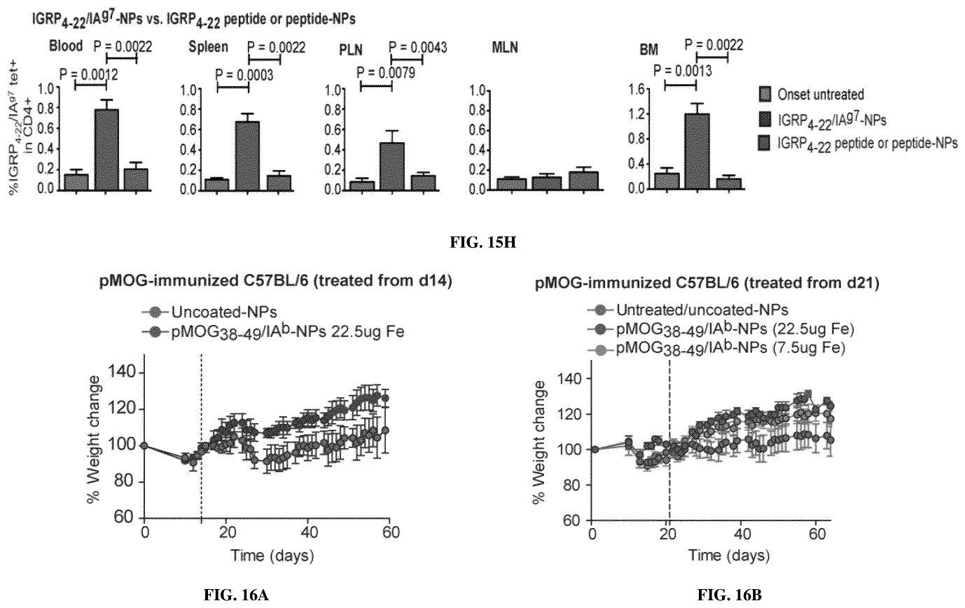

Applicant next investigated whether 2.5mi/IA^{g7}-NPs or NPs coated with IGRP₄₋₂₂/IA^{g7} or IGRP₁₂₈₋₁₄₅/IA^{g7}, targeted by sub-dominant pools of autoreactive CD4⁺ T cells (Mukherjee, R. et al. (2005) J. Immunol. 174:5306-5315), could restore normoglycaemia in diabetic NOD mice. Unlike mice treated with nanoparticles coated with hen egg-white lysozyme (HEL)₁₄₋₂₂/IA^{g7}, 90-100% of the mice that received nanoparticles coated with 2.5mi/IA^{g7}, IGRP₄₋₂₂/IA^{g7} or IGRP₁₂₈₋₁₄₅/IA^{g7} reverted to stable normoglycaemia (FIGS. 9G, 14H) and displayed systemic expansion of cognate T_{R}1-like T cells (FIGS. 1H, 15A-15G). Treatment with peptide (Burton, B. R. et al. (2014) Nature Commun. 5:4741-4747) or peptide-coated nanoparticles but without MHC could not reproduce any of these effects (FIGS. 9G, 14H, 15H). Treatment withdrawal resulted in loss of the normoglycaemic state in 25-60% of mice (FIG. 14I), in association with the loss of the tetramer⁺CD4⁺ T-cell pools (FIGS. 9H, 15A). The animals that maintained normoglycaemia had normal postprandial serum insulin levels, fasting glucose tolerance (FIGS. 14J-14M) and reduced insulitis (FIG. 9I). In addition, their pancreatic lymph nodes (PLNs) could not support the proliferation of carboxyfluorescein succinimidyl ester (CFSE)-labelled IGRP₂₀₆₋₂₁₄/K^{d}-specific CD8⁺ T cells in vivo (FIG. 14N).

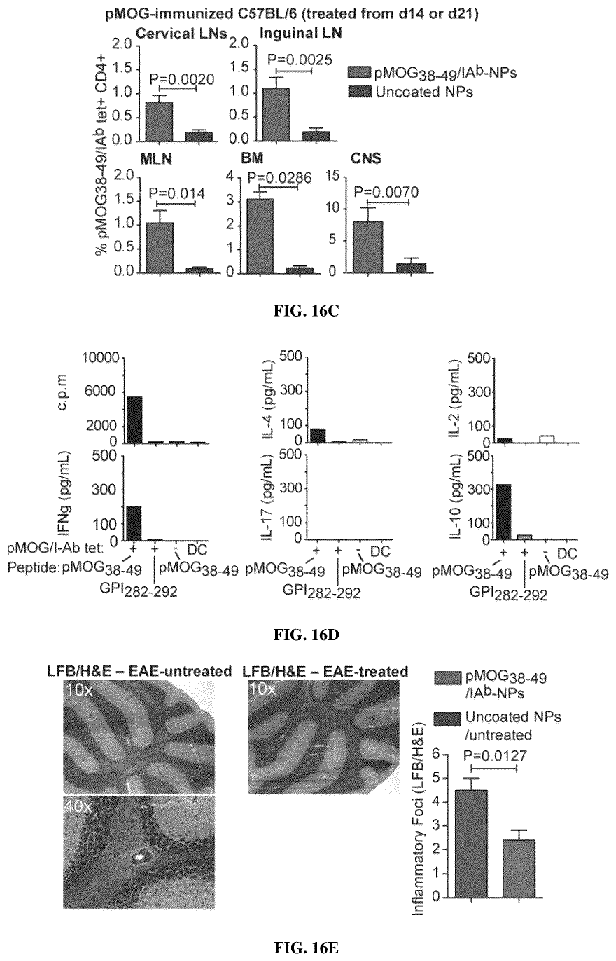

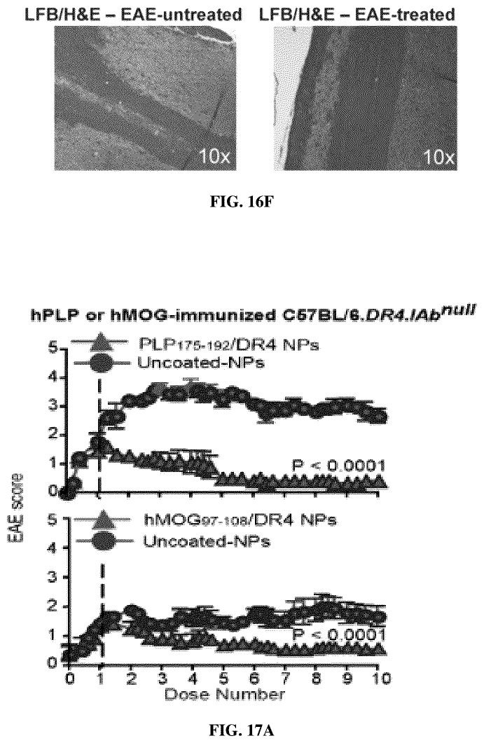

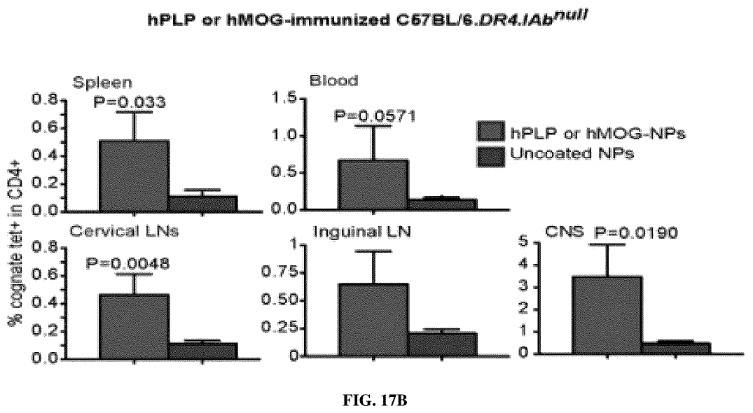

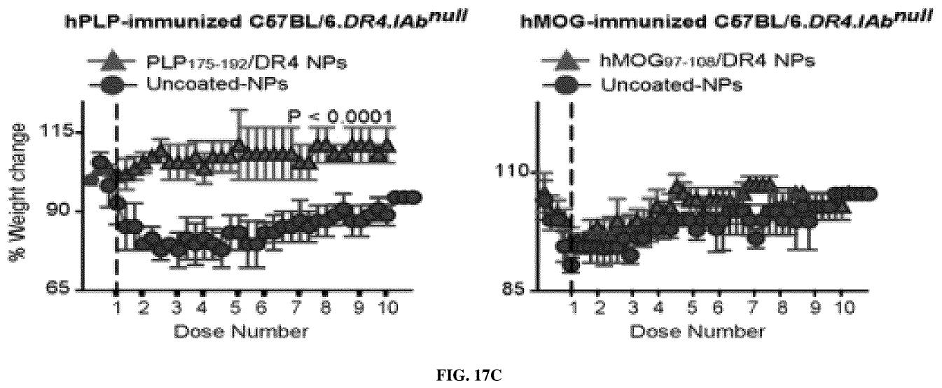

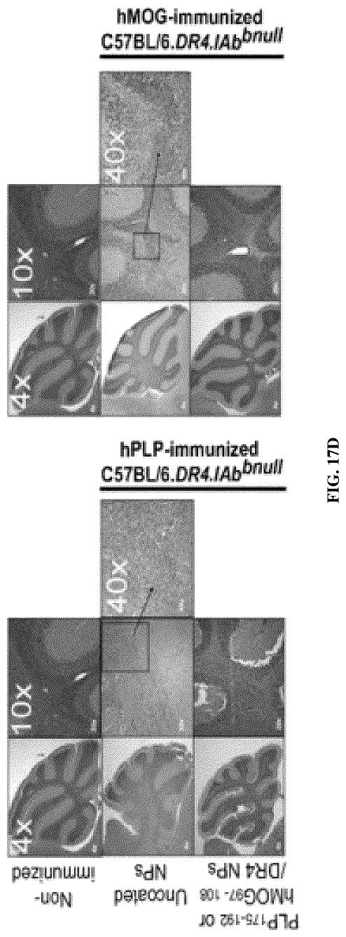

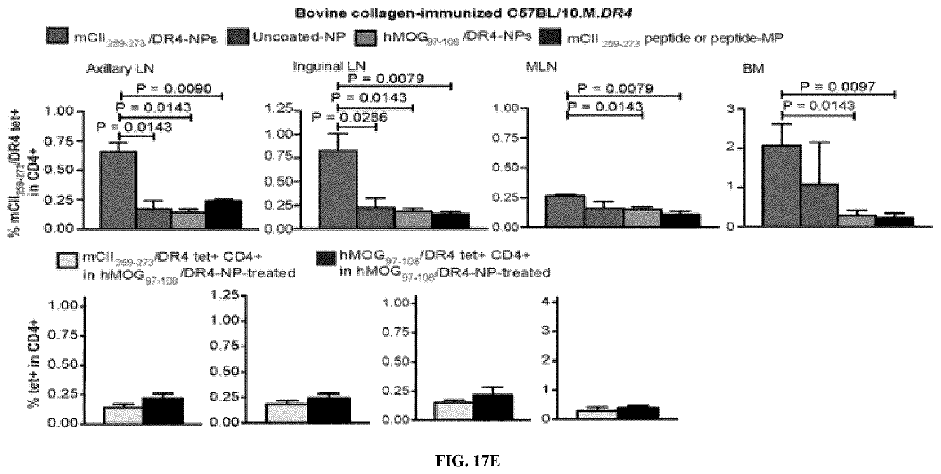

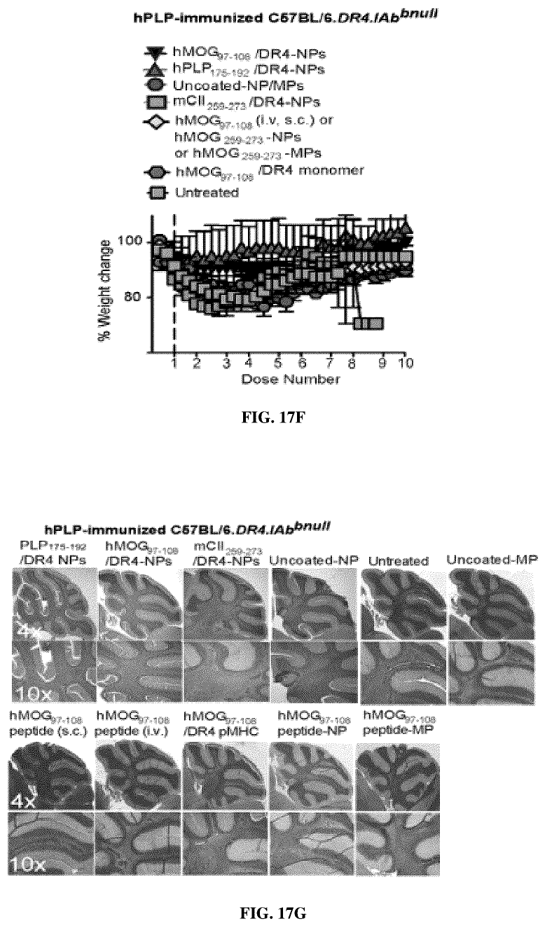

Applicant next tested the ability of nanoparticles coated with myelin oligodendrocyte glycoprotein (pMOG)₃₈₋₄₉/IA^{b} to blunt the progression of pMOG₃₅₋₅₅-induced experimental autoimmune encephalomyelitis (EAE, a model of multiple sclerosis) in C57BL/6 mice. pMOG₃₈₋₄₉/IA^{b}-NP therapy dampended disease progression when given on day 14 after immunization and restored motor function in paralytic mice when given on day 21 (FIGS. 9J, 9K). These effects were mirrored by weight gain, and were associated with systemic expansion of cognate T_{R}1-like T cells, reductions in activated macrophage/microglia in the cerebellum, fewer inflammatory foci and areas of demyelination in the white matter of the cerebellum and decreased demyelination of the spinal cord (FIGS. 9L-9N, 16A-16F). Similar therapeutic effects were seen in HLA-DR4-IE-transgenic C57BL/6 IAb^{null} mice (MHCII knockout mice expressing a transgenic hybrid MHCII molecule composed of the peptide-binding domain of human HLA-DR4 and the membrane-proximal domain of mouse IE (DR4-IE)) immunized with human (h) proteolipid protein (hPLP)₁₇₅₋₁₉₂ or hMOG₉₇₋₁₀₈ peptides and treated with hPLP₁₇₅₋₁₉₂/DR4-IE or hMOG₉₇₋₁₀₈/DR4-IE-NPs upon developing limb paralysis (FIGS. 17A-17D).

### Disease and Organ Specificity

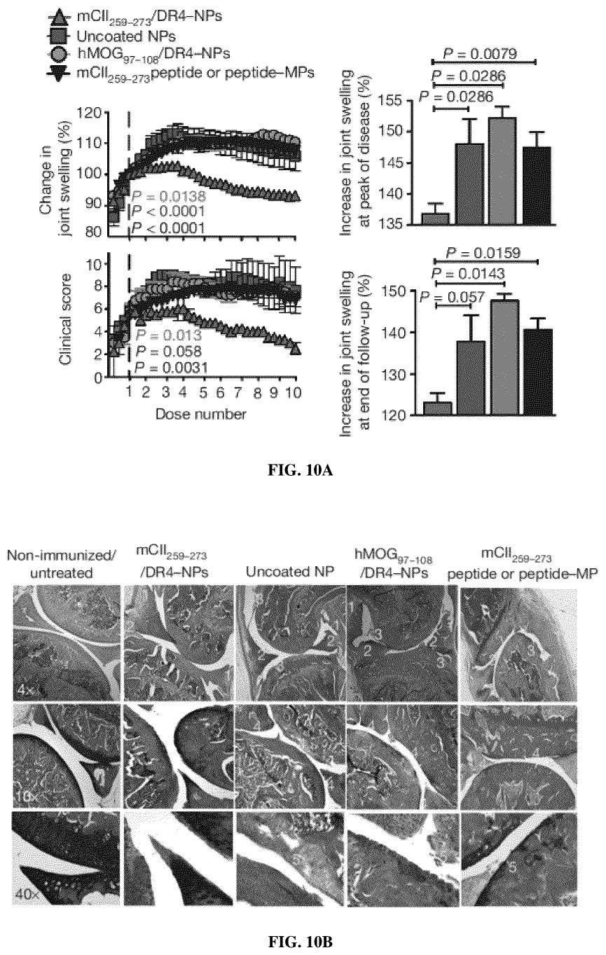

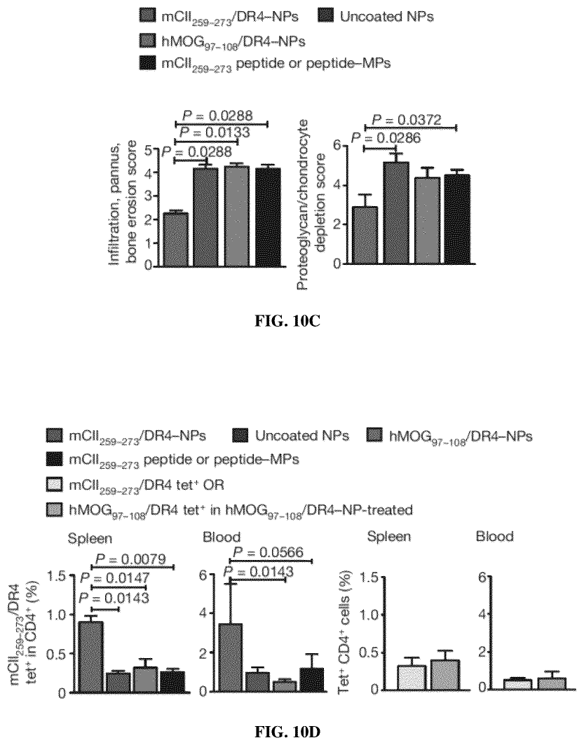

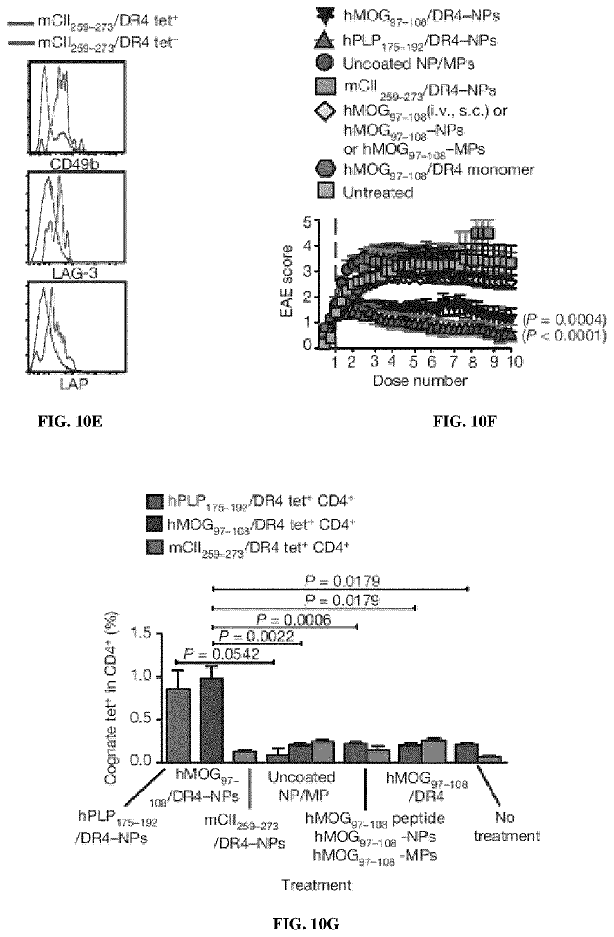

Studies in another autoimmune disease model, collagen-induced arthritis (CIA), showed that nanoparticles displaying mouse collagen (mCII)₂₅₉₋₂₇₃/DR4-IE could reduce joint inflammation in arthritic HLA-DR4-IE-transgenic C57BL/10.M mice in association with systemic expansions of cognate T_{R}1-like T cells (FIGS. 10A-10E, 1E). In contrast, nanoparticles coated with hMOG₉₇₋₁₀₈/DR4-IE complexes had no effect (FIGS. 10A-10C).

To investigate further the disease-specificity of pMHC-NP therapy, Applicant induced EAE in C57BL/6 IAb^{null} HLA-DR4-IE-transgenic mice by immunization with hPLP₁₇₅₋₁₉₂ and treated diseased mice with hPLP₁₇₅₋₁₉₂/DR4-IE-NPs (positive control), uncoated nanoparticles (negative control), EAE-relevant hMOG₉₇₋₁₀₈/DR4-IE-NPs (which display a peptide from a CNS autoantigen other than that used to induce disease), or CIA-relevant mCII₂₅₉₋₂₇₃/DR4-IE-NPs. Whereas hMOG₉₇₋₁₀₈/DR4-IE-NPs blunted EAE as efficiently as the positive control, mCII₂₅₉₋₂₇₃/DR4-IE-NPs had no therapeutic activity (FIGS. 10F, 17F, 17G). Here, too, therapeutic activity was associated with systemic expansions of cognate T_{R}1-like T cells (FIGS. 10G, 10H). Administration of mCII₂₅₉₋₂₇₃ peptide (Burton, B. R. et al. (2014) Nature Commun. 5:4741-4747) or of mCII₂₅₉₋₂₇₃-peptide-coated microparticles (MPs) (Getts, D. R. et al. (2012) Nature Biotechnol. 30:1217-1224) to arthritic C57BL/10.M HLA-DR4-IE-transgenic mice (FIGS. 10A-10D, 17E), or of hMOG₉₇₋₁₀₈ peptide, hMOG₉₇₋₁₀₈/DR4-IE monomers or hMOG₉₇₋₁₁₀-coated nanoparticles or microparticles to C57BL/6 IAb^{null} HLA-DR4-IE-transgenic mice failed to both expand cognate T_{R}1-like cells and blunt disease (FIGS. 10F, 10G, 17F-17I). Thus, the biological and therapeutic effects of pMHCII-NPs are disease-specific and dissociated from the pathogenic role of epitopes (disease-triggering versus downstream autoantigenic targets), suggesting that these compounds act on pre-activated autoreactive T cells and can generate T_{R}1-like cell expansions from rare T-cell precursor pools.

### Soluble Mediators

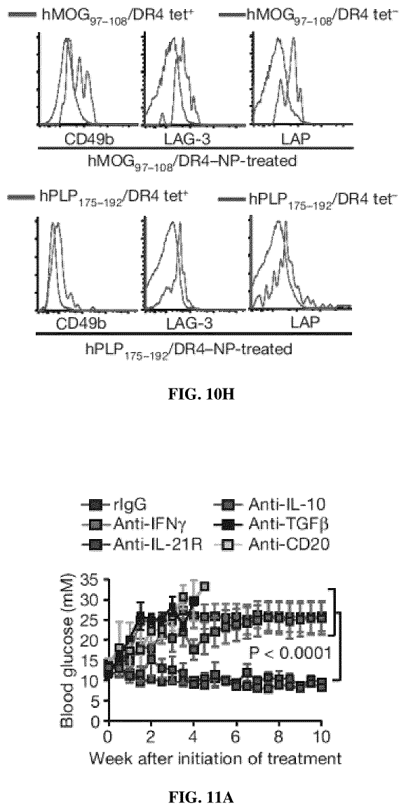

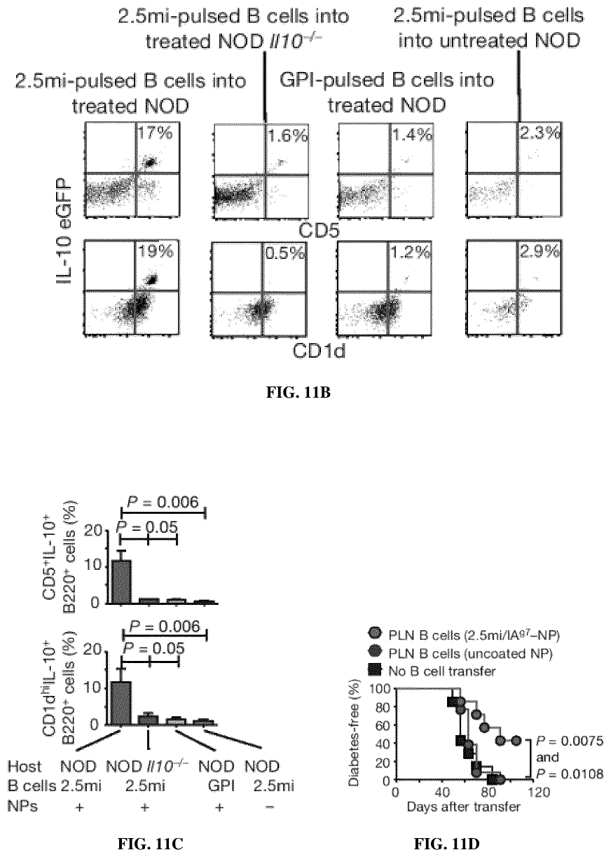

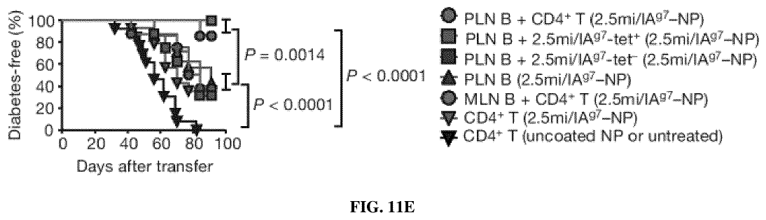

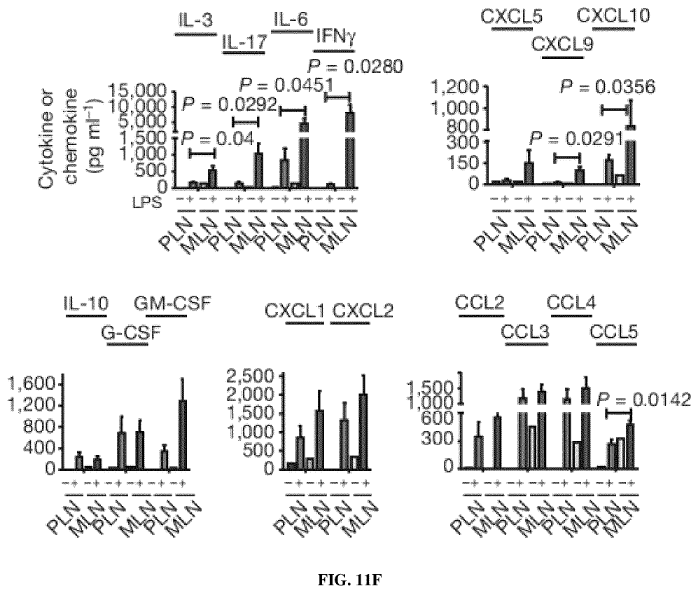

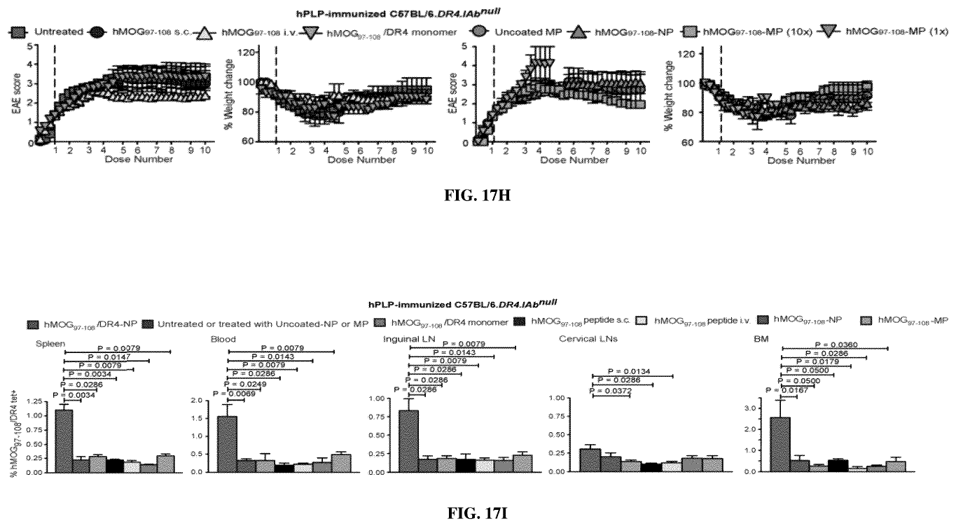

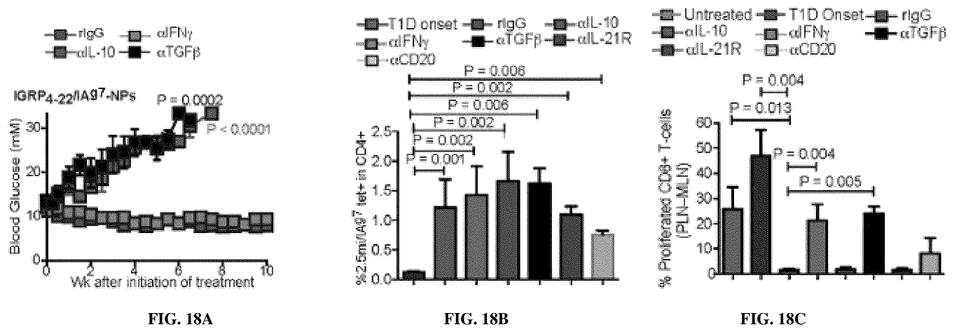

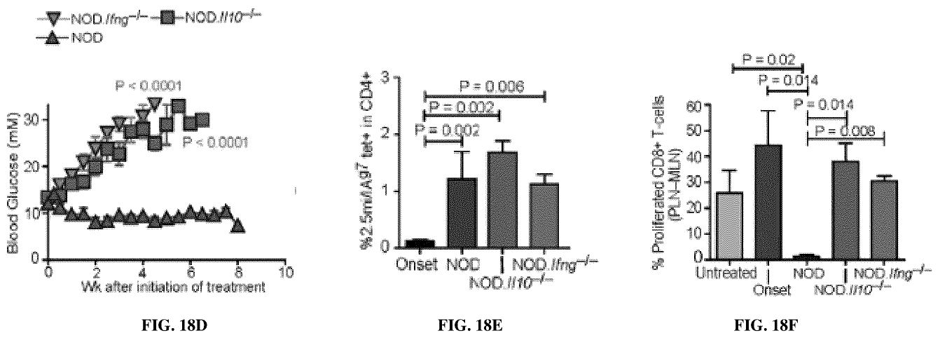

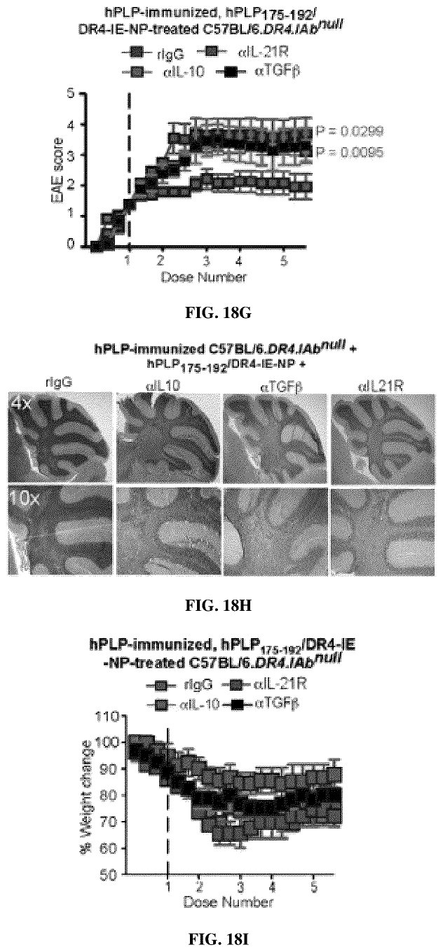

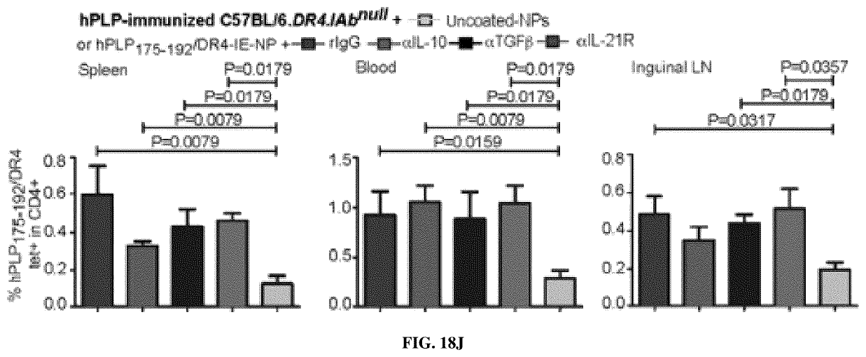

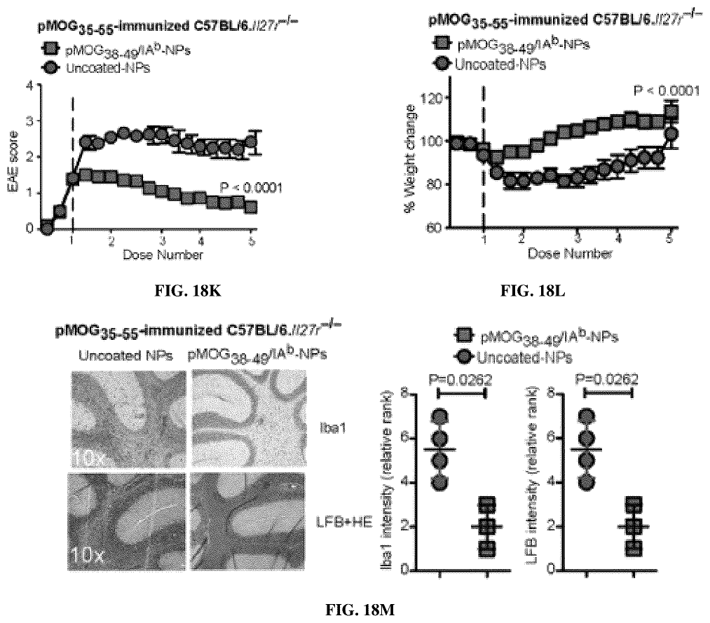

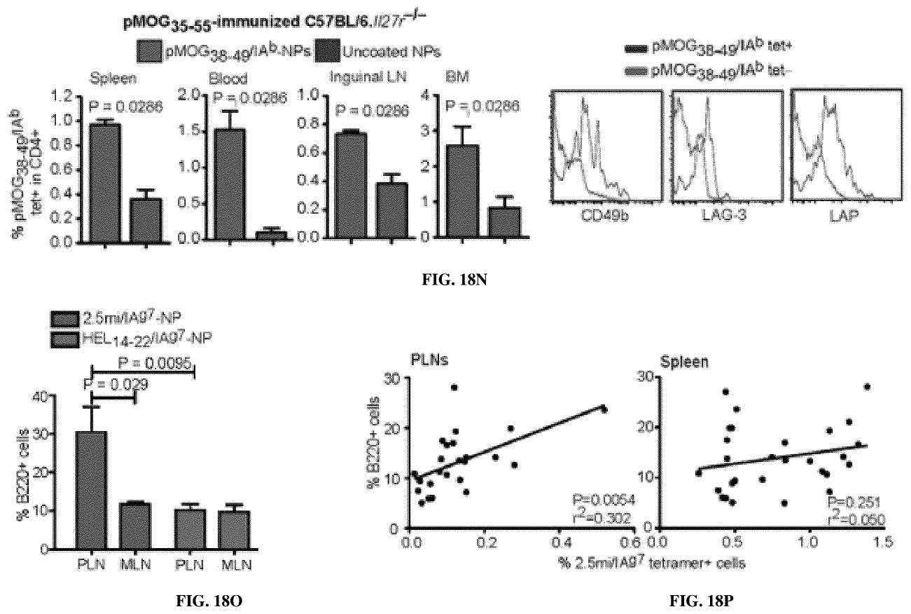

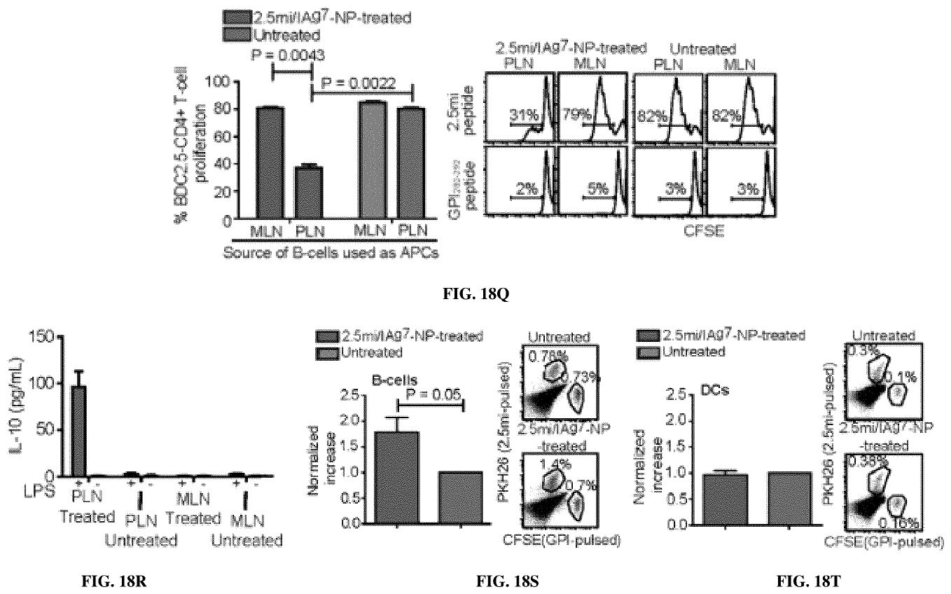

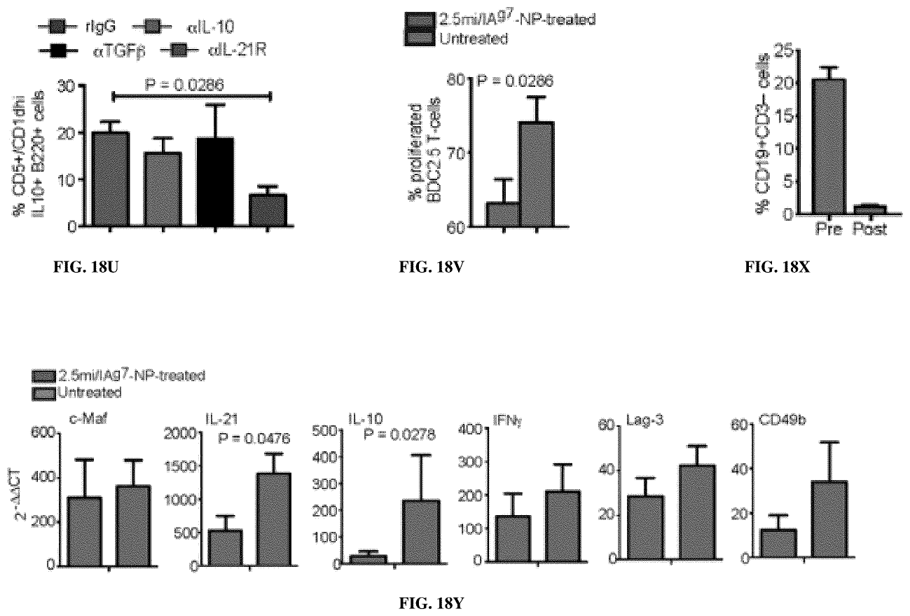

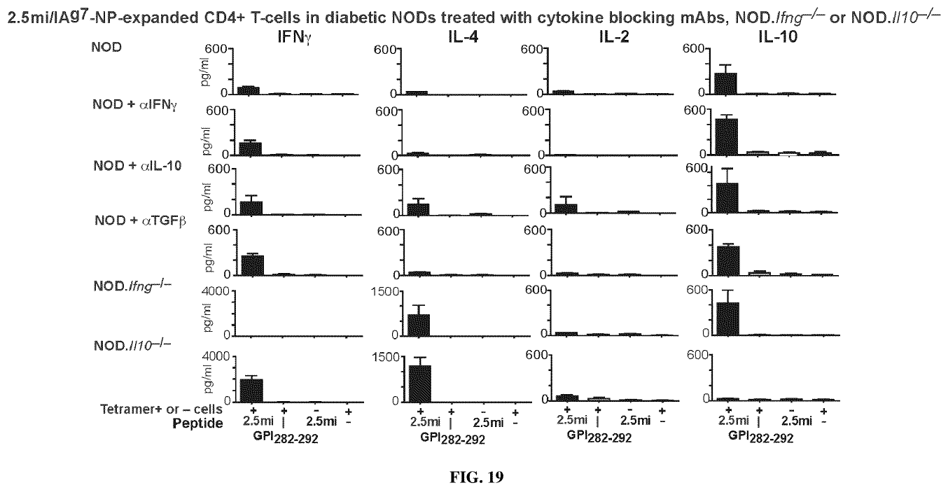

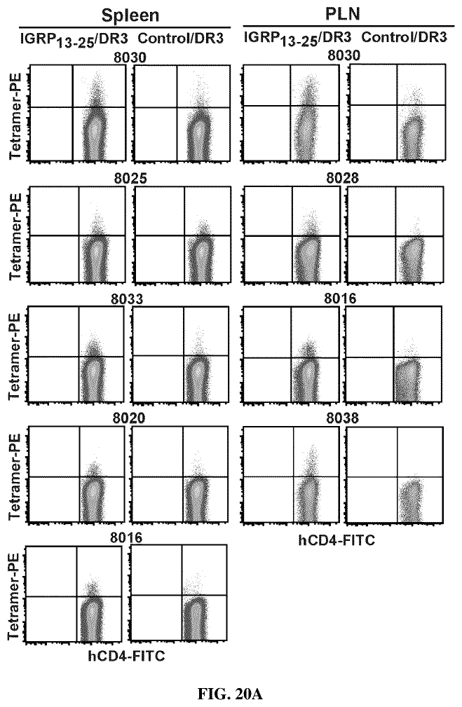

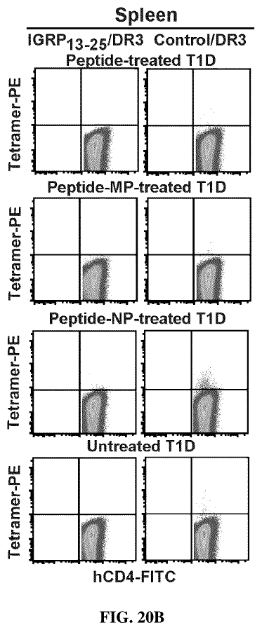

Blockade of IL-10, TGF-β and IL-21R (but not IFNγ) abrogated the anti-diabetogenic properties of 2.5mi/IA^{g7}-NPs or IGRP-22/IA^{g7}-NPs (FIGS. 11A, 18A). With the exception of IL-21R blockade (known to inhibit CD8⁺ T-cell activation), these interventions also abrogated the suppression of autoantigen crosspresentation by the pMHC-NP-expanded T_{R}1-like T cells in the PLNs (FIGS. 18B, 18C, 19). Studies using diabetic NOD Ifng^{-/-} and NOD Il10^{-/-} mice revealed that development of the T_{R}1 precursors and/or T_{R}1-like cells that expand in response to therapy requires IFNγ in addition to IL-10 (FIGS. 18D-18F, 19). mAbs against IL-10, TGF-β and IL-21R also abrogated the anti-encephalitogenic activity of hPLP₁₇₅₋₁₉₂/DR4-IE-NPs in C57BL/6 IAb^{null} HLA-DR4-IE-transgenic mice (FIGS. 18G-18J). pMOG₃₅₋₅₅-immunized C57BL/6 Il27r^{-/-} mice responded to pMOG₃₈₋₄₉/IA^{b}-NPs like their wild-type counter parts (FIGS. 9J-9N, 18K-18N). Thus, IFNγ and IL-10, but not IL-27 (Pot, C. et al. (2009) J. Immunol. 183:797-801), are necessary for pMHC-NP-induced T_{R}1-like cell development; and autoreactive T_{R}1-like T cells use IL-10, TGF-β and IL-21 (but not IFNγ) to suppress disease.

### Downstream Effectors and Network Formation

The PLNs (but not the mesenteric lymph nodes (MLNs) or spleens) of pMHC-NP-treated NOD mice harboured increased percentages of B cells compared with the PLNs of mice treated with pMHCII-NPs not relevant for TID (FIG. 18O). Studies of mice treated with a range of pMHC-NP doses revealed that the sizes of the PLN (but not splenic) B-cell and T_{R}1-like cell pools were correlated (FIG. 18P). Unlike their splenic or MLN counterparts, the PLN B cells of these mice could not effectively present peptide to cognate CD4⁺ T cells ex vivo (FIG. 18Q). In addition, these cells produced IL-10 in response to lipopolysaccharide (LPS) (FIG. 18R), suggesting that pMHC-NP-induced T_{R}1-like cells might trigger the formation and expansion of regulatory B (B_{reg}) cells in the PLNs. In fact, 2.5mi-pulsed B cells, but not DCs, underwent expansion in 2.5mi/IA^{g7}-NP-treated hosts within a week of transfer (FIGS. 18S, 18T).

To probe this further, Applicant transfused NOD Il10^{GFP} splenic B cells that were either pulsed with 2.5mi or a negative control peptide (GPI₂₈₂₋₂₉₂), into 2.5mi/IA^{g7}-NP-treated NOD or NOD Il10^{-/-} hosts. Seven days later, the hosts were analysed for the presence of IL-10-producing (eGFP⁺) CD5⁺CD1d^{high} B cells. NOD (but not NOD Il10^{-/-}) mice treated with 2.5mi/IA^{g7}-NPs efficiently induced formation of B_{reg} cells specifically from 2.5mi-pulsed B cells, and IL-21R but not IL-10 or TGF-β blockade suppressed this effect (FIGS. 11B, 11C, 18U).

In vitro, the PLN B cells of 2.5mi/IA^{g7}-NP-treated mice had a moderate suppressive effect on the proliferative activity of BDC2.5 CD4⁺ T cells in response to peptide-pulsed DCs (FIG. 18V). In vivo, these B cells suppressed diabetes development in T-cell-reconstituted NOD scid hosts as compared to PLN B cells from control mice (FIG. 11D). Co-transfer of PLN B cells and bulk or 2.5mi/IA^{g7}-tetramer+splenic CD4⁺ T cells from 2.5mi/IA^{g7}-NP-treated mice resulted in >95% suppression, as compared to PLN B cells with or without tetramer-CD4⁺ T cells from 2.5mi/IA^{g7}-NP-treated mice, to CD4⁺ T cells with or without MLN B cells from 2.5mi/IA^{g7}-NP-treated mice (˜40%), or to CD4+ T cells from untreated or control-NP-treated mice (0%) (FIG. 11E), supporting synergistic effects. In agreement with this, treatment of newly diabetic NOD mice with a B-cell-depleting anti-CD20 mAb abrogated the anti-diabetogenic activity of 2.5mi/IA^{g7}-NPs (FIGS. 11A, 18X).

Comparison of the cytokine and chemokine profile of CD11b⁺ cells derived from the PLN or MLN of pMHC-NP-treated NOD mice further revealed that CD11b⁺ cells from the PLN produced lower levels of the pro-inflammatory mediators IL-3, IL-17, IL-6, IFNγ, CXCL9 and CXCL10 in response to LPS than their MLN counterparts did (FIG. 11F). Importantly, the effects of pMHC-NP therapy on antigen-presenting cells (APC)s from draining lymph nodes were not associated with impaired systemic immunity because pMHC-NP-treated NOD mice cleared an acute viral infection and mounted antibodies against an exogenous antigen as efficiently as untreated mice (FIGS. 11G, 11H).

### Antigen-Experienced T Cells as Targets

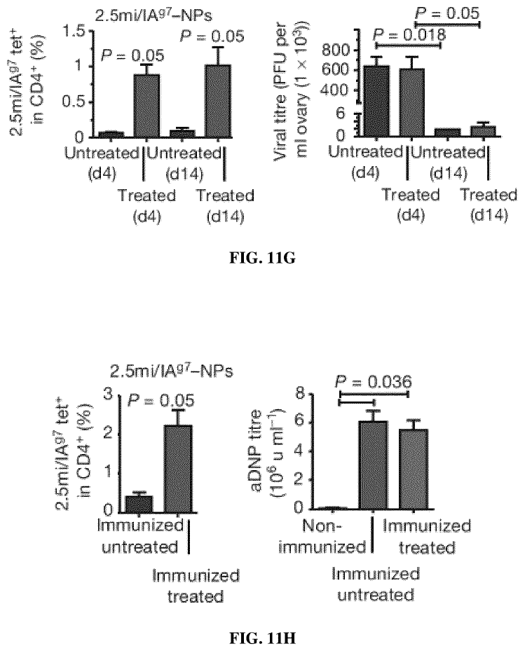

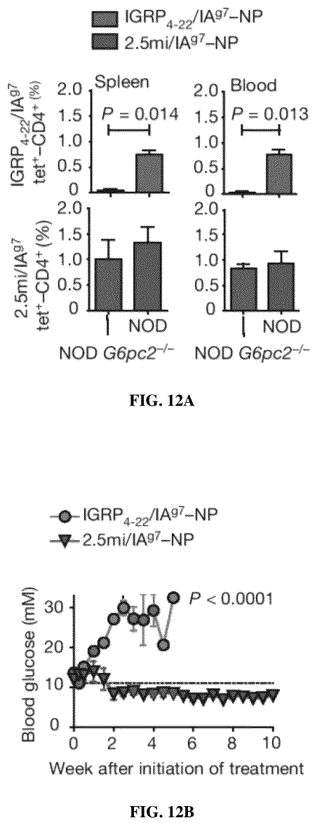

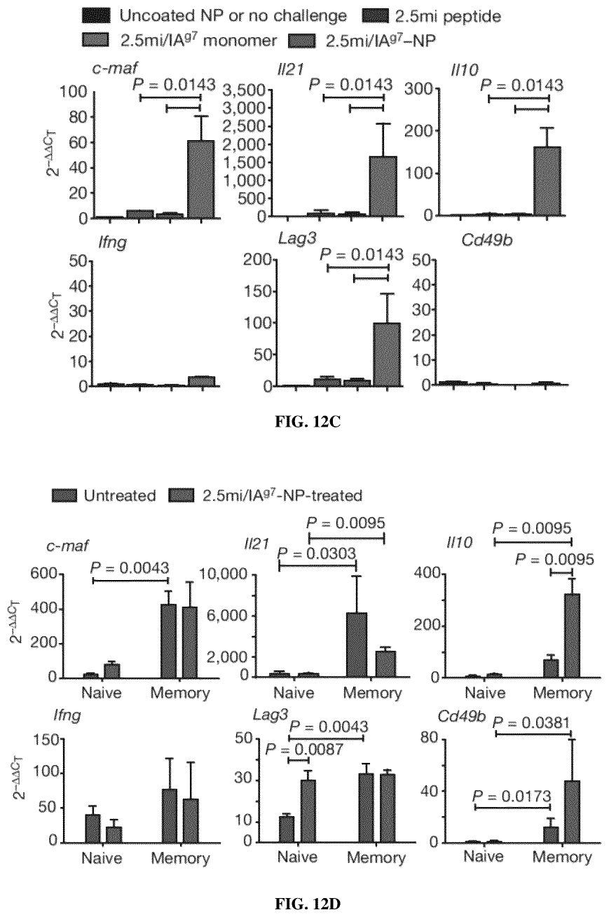

The memory-like phenotype and the upregulation of T-bet mRNA in the expanded T_{R}1-like cells, coupled with the inability of pMHC-NPs to expand cognate T_{R}1-like cells in non-diseased mice or NOD Ifng^{-/-} mice suggested that pMHC-NPs expand pre-existing T_{R}1 cells that arise from antigen-experienced precursors; and/or trigger the differentiation of antigen-experienced T_{H}1 cells into T_{R}1-like cells. Indeed, whereas diabetic NOD G6pc2^{-/-} mice (which lack IGRP) responded to 2.5mi/IA^{g7}-NPs like wild-type NOD mice, they did not respond to IGRP₄₋₂₂/IA^{g7}-NPs (FIG. 12A, 12B). In vitro, 2.5mi/IA^{g7}-NPs triggered the expression of CD49b and LAG-3 and the upregulation of c-maf, Il21, Il10 and Lag3 mRNA exclusively in anti-CD3 plus anti-CD28 mAb-activated, but not naive BDC2.5 CD4⁺ T cells (FIGS. 12C, 14D).

To investigate this further, Applicant transfused naive (CD441^{low}CD62L^{hi}) or memory-like (CD44^{hi}CD62L^{low}) BDC2.5 CD4⁺ T cells into hosts of the congenic NOD.Thy1^{a} strain and measured changes in their expression of LAG-3 and CD49b protein and c-maf, Il21, Il10, Ifng, Lag3 and Cd49b mRNA, both upon 2.5mi/IA^{g7}-NP therapy and in the absence of therapy. Notably, the memory T cells from pMHC-NP-untreated hosts expressed about one hundred-fold higher levels of c-maf and 1121 and, to a lesser extent, Lag3 and Cd49b, but not 1110 mRNA than their naive counterparts (FIG. 12D). This is in accordance with the observed demethylation of 1121 and the c-Maf/IL-10- and IL-21-expression competency of effector/memory CD4⁺ T cells (Pot, C. et al. (2009) J. Immunol. 183:797-801; Spensieri, F. et al. (2013) Proc. Natl Acad. Sci. USA 110:14330-14335; Hale, J. S. et al. (2013) Immunity 38:805-817; Sato, K. et al. (2011) J. Biol. Chem. 286:14963-14971; Saraiva, M. et al. (2009) Immunity 31:209-219), and suggests that the memory T-cell pool is enriched for uncommitted T_{R}1 precursors, expressing a T_{R}1-poised transcriptional program. Remarkably, whereas pMHC-NP therapy only upregulated Lag3 mRNA and, to a lesser extent, LAG-3 protein in naive BDC2.5 CD4⁺ T cells, it promoted the upregulation of Il10 mRNA and LAG-3 and CD49b protein, and the proliferation of memory BDC2.5 CD4⁺ T cells (FIGS. 12D-12F). Similar results were observed using memory eGFP⁻ (FOXP3⁻) BDC2.5 CD4⁺ cells from BDC2.5 NOD Foxp3-eGFP mice (FIG. 18Y).

These effects on antigen-experienced T cells were accompanied by acquisition of anti-diabetogenic properties: whereas pMHC-NP therapy afforded 100% diabetes protection to T-cell-reconstituted NOD scid hosts bearing memory BDC2.5 T cells, therapy was inconsequential in hosts receiving naive BDC2.5 T cells (FIG. 12G). Therefore, pMHC-NP therapy promotes the differentiation (and expansion) of c-Maf-expressing antigen-experienced CD4⁺ T cells into T_{R}1 progeny.

### Translational Potential

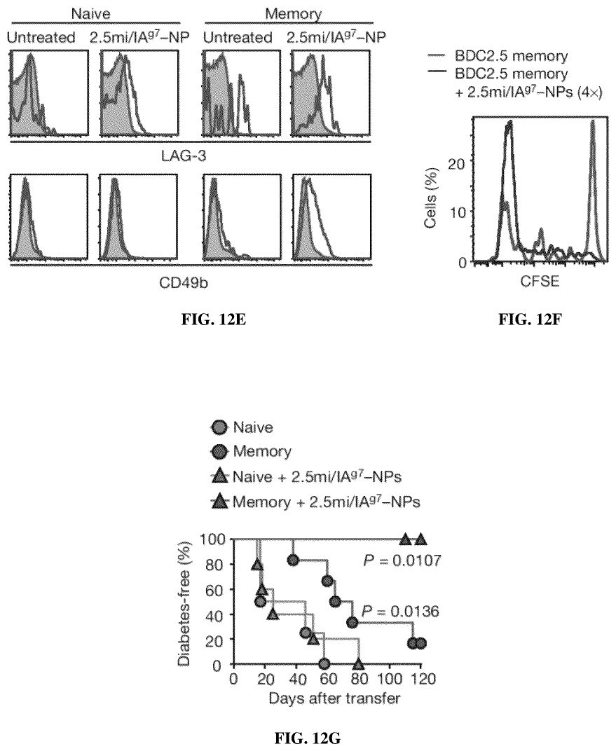

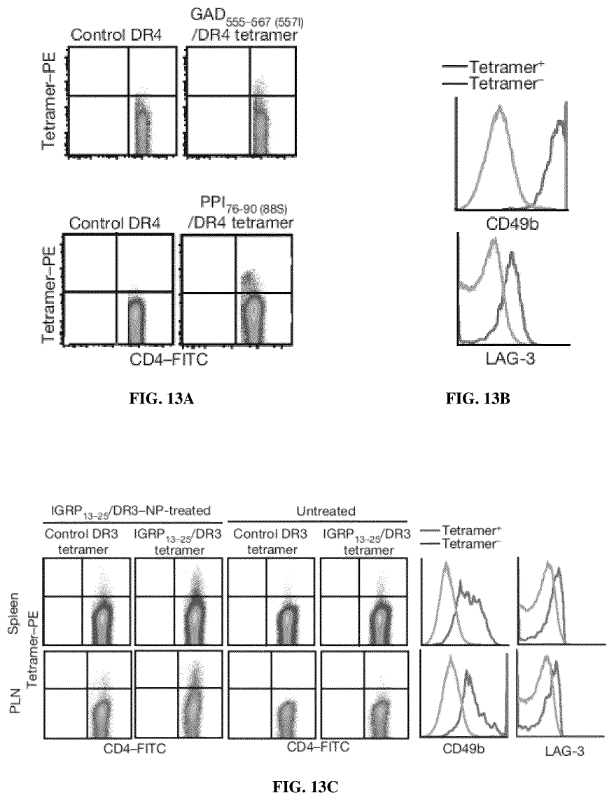

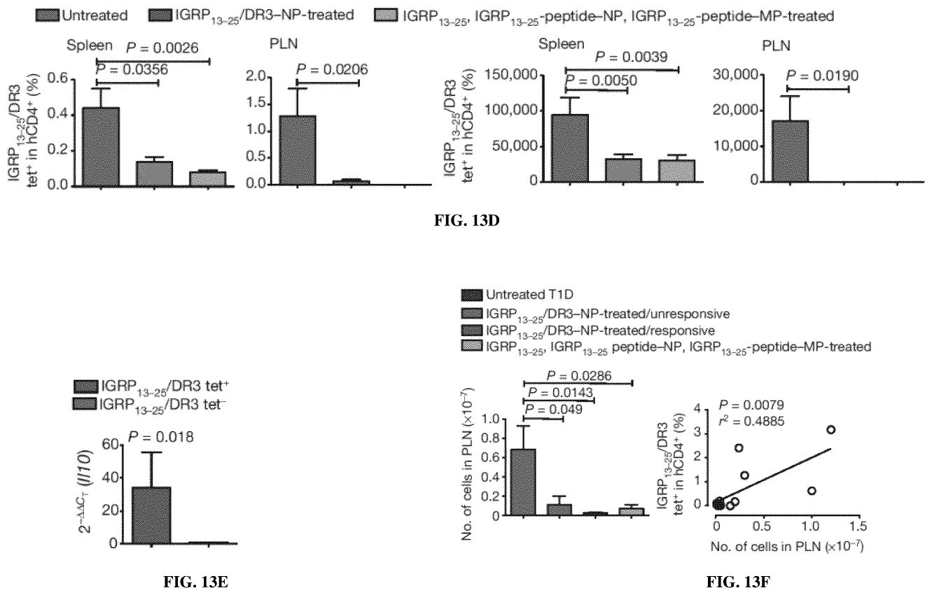

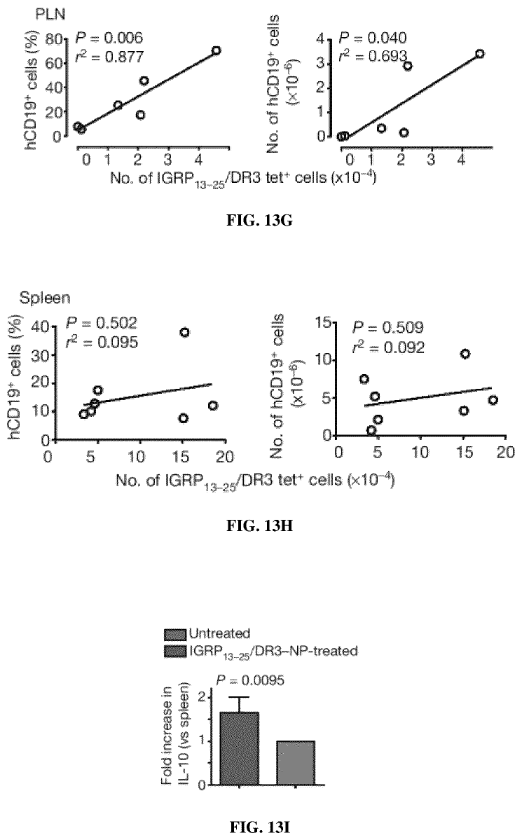

Applicant determined the ability of human T1D-relevant pMHCII-NPs to expand cognate T_{R}1-like T cells in NOD scid I12rg^{-/-} (NSG) hosts reconstituted with peripheral blood mononuclear cells (PBMCs) from T1D patients (Table 2). Initial assay development focused on NSG hosts reconstituted with PBMCs from five DRB1*0401⁺ recent-onset T1D patients and treated with nanoparticles coated in either human glutamic acid decarboxylase-65 (GAD65)₅₅₅₋₅₆₇(F557I)/DR4 or preproinsulin (PPI)76-90(K88S)/DR4 (FIGS. 13A, 13B, Table 2). Applicant then repeated these experiments using NSG hosts reconstituted with PBMCs from 7 DRB1*0301⁺ T1D patients and a third T1D-relevant pMHC-NP type (hIGRP13-25/DR3-NPs) given at a higher dose. Applicant saw expansion of tetramer⁺CD49b⁺LAG-3⁺CD4⁺ T cells in the spleen and/or PLNs (endogenous mouse (m)IGRP₁₃₋₂₅ is highly homologous to hIGRP₁₃₋₂₅) from all seven pMHC-NP-treated mice and none of the untreated controls (FIGS. 13C, 13D, 20A and Table 2). The average percentage and numbers of tetramer⁺CD4⁺ T cells in IGRP13-25/DR3-NP-treated mice were significantly greater than in untreated littermates (FIG. 13D) and expressed Il10 mRNA (FIG. 13E). These responses could not be induced with peptide or peptide-coated nanoparticles or microparticles (FIGS. 13D, 20B and Table 2).

The PLNs of the pMHC-NP-treated mice that harboured increased percentages of tetramer⁺CD4⁺ T cells had increased cellularity (FIG. 13F). Furthermore, there were correlations between the number of PLN tetramer⁺CD4⁺ T cells and the percentage and absolute number of PLN human B cells, and the PLN B cells, unlike their splenic counterparts, produced IL-10 in response to LPS (FIGS. 13G-13I), suggesting B_{reg} formation and/or recruitment. No such responses were seen in patient hPBMC-reconstituted NSG mice treated with peptide or peptide-NPs/MPs (FIG. 13F).

### Discussion

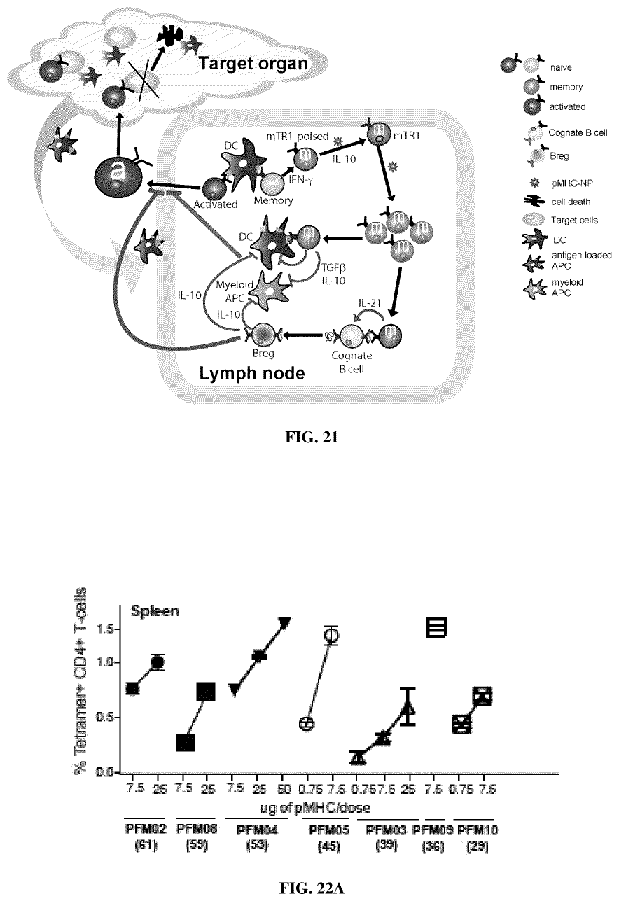

Applicant has shown that systemic therapy with nanoparticles coated with autoimmune-disease-relevant pMHC class II complexes triggers the expansion of cognate T_{R}1-like CD4⁺ T cells, restores normoglycaemia in spontaneously diabetic mice and motor function in paralyzed EAE mice, and resolves joint swelling and destruction in arthritic mice, without compromising systemic immunity. Applicant demonstrates that this outcome is dissociated from genetic background and type of auto immune disease and can be replicated with ten different human or murine autoimmune-disease-relevant pMHC-NP types. The cell surface phenotype, cytokine secretion pattern, transcriptional profile and function of the T_{R}T-like cell pools expanded by pMHCII-based nanomedicines are consistent with those described for murine T_{R}1-like CD4⁺ T cells and remarkably similar to T_{R}1 cells derived from healthy individuals and autoimmune disease patients (Gagliani, N. et al. (2013) Nature Med. 19:739-746). Applicant demonstrates key roles for prior autoantigenic experience and IFNγ—and IL-10-expression competence in the developmental biology of autoreactive T_{R}1 cells. Applicant shows that pMHCII-NPs promote IL-10 transcription and the upregulation of T_{R}T markers in T_{R}1-poised, antigen-experienced CD4⁺ T cells in an APC and IL-27-independent manner, followed by systemic expansion. The need for IFNγ, the expression of the T_{H}T transcription factor T-bet, the c-Maf/IL-10- and IL-21-expression competency of effector and memory CD4⁺ T cells (Pot, C. et al. (2009) J. Immunol. 183:797-801; Spensieri, F. et al. (2013) Proc. Natl Acad. Sci. USA 110:14330-14335; Hale, J. S. et al. (2013) Immunity 38:805-817), and the ability of pMHCII-NPs to turn T cells primed by active immunization into T_{R}1 suppressors suggest that these T_{R}1 precursors are effector/memory T_{H}1 cells. Applicant defines the mechanisms of action and uncover a cascade of cellular interactions downstream of the pMHC-NP-expanded T_{R}1-like cells, including B_{reg} cell formation, that coordinately lead to the resolution of inflammation in an antigendependent but antigen-non-specific manner (FIG. 21).

Collectively, Applicant's data support the contention that any single pMHC involved in a given autoimmune disease could be used to blunt complex autoimmune responses via this approach. Consistent with this prediction, the 20 pMHCI/II-based nanomedicines tested to date have similar efficacy, regardless of antigen prevalence, dominance or role in the disease process. Neither pMHC monomers nor peptides or peptide-coated nanoparticles/microparticles trigger cognate T_{R}1 cell formation/expansion from the polyclonal T-cell repertoires or reverse T1D, CIA or EAE in the chronic models tested here. pMHC-based nanomedicines thus represent a new class of therapeutics in autoimmunity, capable of resolving cellularly and antigenically complex autoimmune responses in a disease- and organ-specific manner without compromising systemic immunity.

### Methods

### Mice.

NOD/Ltj, NOD scid, BDC2.5-NOD, NOD Il10^{-/-}, C57BL/6, C57BL/6 Il27r^{-/-}, C57BL/10.M, NOD Foxp3-egfp and NOD scid I12rg^{-/-}(NSG) mice were purchased from the Jackson Lab. NOD Ifng^{-/-} and LCMV Gp33-specific TCR-transgenic NOD mice were from D. Serreze (Jackson Lab). HLA-DR4-IE-transgenic C57BL/6 IAb^{null} mice were purchased from Taconic Farms. NOD Il10^{GFP} (tiger) mice were obtained by backcrossing the Il10^{GFP} allele from C57BL/6 Il10^{GFP} mice (Jackson Lab) onto the NOD/Ltj background for 10 generations. 8.3-NOD and NOD G6pc2^{-/-} mice have been described elsewhere (Verdaguer, J. et al. (1997) J. Exp. Med. 186:1663-1676; Wang, J. et al. (2010) Proc. Natl Acad. Sci. USA 107:9317-9322). These studies were approved by the corresponding institutional animal care committees. No statistical methods were used to predetermine sample size.

### Antibodies, Tetramer Staining and Flow Cytometry.

FITC, PE, PerCP or biotin-conjugated mAbs against mouse CD4 (RM4-5), CD8α (53-6.7), B220 (RA36B2), CD62L (MEL-1), CD69 (H1.2F3), CD44 (IM7), and CD49b (DX5) and streptavidin-PerCP were purchased from BD Pharmingen. The antibody against murine LAG-3 (C9B7W) was from eBioscience. Anti-latent-associated-TGF-β antibody (TW7-16B4) was from BioLegend. PE-conjugated pMHC class II tetramers were prepared using biotinylated pMHC monomers. Peripheral blood mononuclear cells, splenocytes, lymph node and bone marrow CD4⁺ T cells were incubated with avidin for 15 min at room temperature and stained with tetramer (5 μg ml-1) in FACS buffer (0.05% sodium azide and 1% FBS in PBS) for 30-120 minutes at 4° C. or 37° C., depending on the tetramer, washed, and incubated with FITC-conjugated anti-CD4 (5 μg ml⁻¹) and PerCP-conjugated anti-B220 (2 μg ml⁻¹; as a ‘dump’ channel) for 30 min at 4° C. Cells were washed, fixed in 1% paraformaldehyde (PFA) in PBS and analysed with FACScan, FACSaria, or BD LSRII flow cytometers. For other phenotypic analyses, single-cell suspensions were stained with pMHC tetramers and antibodies diluted 1:100 in FACS buffer (all used at 4° C. except anti-LAG-3, which was used at 37° C.), washed, fixed in 1% PFA, and analysed by FACS. All phenotypic staining were performed in the presence of an anti-CD16/CD32 mAb (2.4G2; BD Pharmingen) to block Fc receptors. Analysis was done using FlowJo software.

NSG-engrafted human T cells were analysed using the following mAbs: FITC-conjugated anti-CD4 (OKT4, BioLegend), APC-conjugated anti-CD19 (HIB19, BD Pharmingen), PerCP-conjugated polyclonal goat anti-LAG-3 IgG (R&D Systems), biotin-conjugated anti-CD49b (AK7, Pierce Antibodies, Thermo Scientific), and EF450-conjugated streptavidin (eBioscience). Briefly, splenocytes and pancreatic lymph node cells were incubated with avidin (0.25 mg ml⁻¹ in FACS buffer) for 30 min at room temperature, washed and stained with tetramer (5 μg ml⁻¹) for 1 hour at 37° C., washed, and incubated with FITC-conjugated anti-CD4 (2/100), APC-conjugated anti-CD19 (5/100; used as a ‘dump’ channel), PerCP-conjugated anti-LAG-3 (8/100) and biotin-conjugated anti-CD49b (4/100) at 4° C. for 45 minutes. After washing, the cells were incubated with EF450-conjugated streptavidin for 30 minutes at 4° C., washed, fixed in 1% PFA in PBS and cells within the hCD4⁺/hCD19⁻ gate analysed with a FACSCanto II (BD Bioscience).

Peptides and pMHCs.

Unless specified otherwise, recombinant pMHC class II monomers were purified from culture supernatants of induced Drosophila SC2 cells transfected with constructs encoding I-Aβ and I-Aα chains carrying c-Jun or c-Fos leucine zippers, respectively, and a BirA and 6× His tags. In these constructs, the peptide-coding sequence was tethered to the amino-terminal end of the I-Aβ chain via a flexible Gly-Ser linker as described (Stratmann, T. et al. (2003) J. Clin. Invest. 112:902-914). GAD65_{555(557I)-567}/DR4, PPI_{76-90(88S)}/DR4 and IGRP₁₃₋₂₅/DR3 monomers were produced by loading the corresponding peptides onto DR4 and DR3 complexes purified from supernatants of induced SC2 cells, as described (Yang, J. et al. (2006) J. Immunol. 176:2781-2789). Other constructs (those encoding 2.5mi/IA^{g7}, pMOG₃₅₋₅₅/IA^{b}, hMOG₉₇₋₁₀₈/DR4-IE, hPLP₁₇₅₋₁₉₂/DR4-IE and mCII₂₅₉₋₂₇₃/DR4-IE) were purified from supernatants of Chinese Hamster Ovary (CHO) cells transduced with lentiviruses encoding a monocistronic message in which the peptide-MHCβ and MHCα chains of the complex were separated by the ribosome skipping P2A sequence (Holst, J. et al. (2006) Nature Protocols 1:406-417). These monomers were engineered to encode a BirA site, a 6× His tag and a free Cys at the carboxyterminal end of the construct. The self-assembled pMHC class II complexes were purified by nickel chromatography and used for coating onto nanoparticles or processed for biotinylation and tetramer formation as described above. The epitopes encoded in the different monomeric constructs used here include: 2.5mi; AHHPIWARMDA) (Stratmann, T. et al. (2003) J. Clin. Invest. 112:902-914); IGRP₁₂₈₋₁₄₅ (TAALSYTISRMEESSVTL) and IGRP₄₋₂₂ (LHRSGVLIIHHLQEDYRTY) (15); HEL₁₄₋₂₂ (RHGLDNYRG); GAD65_{555(557I)-567} (NFIRMVISNPAAT) (Reijonen, H. et al. (2002) Diabetes 51:1375-1382); PPI₇₆₋₉₀(88S) (SLQPLALEGSLQSRG) (Yang, J. et al. (2008) J. Autoimmun. 31:30-41); IGRP₁₃₋₂₅ (QHLQKDYRAYYTF) (Yang, J. et al. (2006) J. Immunol. 176:2781-2789); pMOG₃₈₋₄₉ (GWYRSPFSRVVH); hMOG₉₇₋₁₀₈ (TCFFRDHSYQEE); hPLP₁₇₅₋₁₉₂ (YIYFNTWTTCQSIAFPSK); and mCII₂₅₉₋₂₇₃ (GIAGFKGDQGPKGET). IGRP₄₋₂₂, IGRP₁₂₈₋₁₄₅ and GPI₂₈₂₋₂₉₂ (LSIALHVGFDH) or 2.5mi, pMOG₃₅₋₅₅ (MEVGWYRSPFSRVVHLYRNGK), pMOG₃₈₋₄₉, hMOG₉₇₋₁₀₈ and hPLP₁₇₅₋₁₉₂ peptides were purchased from Sigma Genosys, Mimotopes or Genscript.

Nanoparticles, pMHC-NP, Peptide-NP and Peptide-MP Synthesis and Purification.

Applicant coated pMHCs onto crosslinked dextran-coated or pegylated iron oxide NPs (CLIO- or PFM-NPs, respectively). Briefly, CLIO-NPs were treated with ammonia to produce amino groups (NH₂). Avidin was oxidized with sodium periodate and added to the amino-NPs. Further incubation with sodium cyanoborohydride was used to generate a stable covalent bond. Finally, biotinylated monomers were added to the nanoparticles at a molar ratio of 4 mol biotin/mol avidin (Moore, A. et al. (2004) Diabetes 53:1459-1466). PFM-NPs were produced by thermal decomposition of Fe(acac)₃ in the presence of 2 kDa methoxypolyethylene glycol maleimide (Singha, S. et al., unpublished data). The NPs were purified using magnetic (MACS) columns (Miltenyi Biotec) or an IMag cell separation system (BD BioSciences). To conjugate pMHC or free peptide to PFM-NPs, we incubated pMHCs or peptide carrying a free carboxyterminal Cys with nanoparticles in 40 mM phosphate buffer, pH 6.0, containing 2 mM EDTA, 150 mM NaCl overnight at room temperature. The pMHC-conjugated nanoparticles were separated from free pMHC or peptide using magnetic columns, sterilized by filtration through 0.2 μm filters and stored in water or PBS at 4° C. Quality control was performed using transmission electron microscopy, dynamic light scattering, and native and denaturing gel electrophoresis. pMHC or peptide content was measured using different approaches, including Bradford assay (Thermo Scientific), denaturing SDS-PAGE, amino acid analysis (HPLC-based quantification of 17 different amino acids in hydrolyzed pMHC-NP preparations) or dot-ELISA (Singha, S. et al., unpublished data).

Peptide-coated microparticles were made using carboxylated 500 nm diameter polystyrene beads from Polysciences (Warrington, PA) as previously described (Getts, D. R. et al. (2012) Nature Biotechnol. 30:1217-1224). The peptides were conjugated to polystyrene beads via carbodiimide chemistry following the manufacturer's instructions. Briefly, Applicant incubated 250 μl PSB (containing ˜9×10¹¹ beads) with 250 μg peptide in 0.1 M MES buffer, pH 5.0 at room temperature with gentle rolling in the presence of 1 mg EDC for 2 hours. The peptide-conjugated polystyrene beads were washed with PBS to remove unconjugated peptides and analysed with native and denaturing PAGE against serial dilutions of unconjugated peptide and microparticle controls.

pMHC-NP and Peptide or Peptide-NP Therapy in NOD Mice.

Experiments in pre-diabetic NOD mice involved treating (i.v.) cohorts of 10-week-old female mice with 7.5 μg of pMHC-NPs, or equivalent amounts of soluble pMHC monomers or uncoated nanoparticles twice weekly for 5 consecutive weeks. Experiments in diabetic mice involved following cohorts of 10-week-old female NOD/Ltj, NOD G6pc2^{-/-}, NOD Il10^{-/-} or NOD Ifng^{-/-} mice for diabetes development by measuring blood glucose levels with Accucheck Strips (Roche) twice a week. Mice displaying two consecutive measurements >11 mM were considered diabetic and treated twice weekly with 7.5 μg pMHC-NPs, nanoparticles delivering a molecular equivalent of peptide or free peptide (8 μg per dose) (Burton, B. R. et al. (2014) Nature Commun. 5:4741-4747), until stably normoglycaemic (defined as 8 consecutive measurements <11 mM) or until hyperglycaemia was considered irreversible (3 measurements >25 mM). In FIGS. 9G, 12B, and 14H, mice were randomized into treatment with 2.5mi/IA^{g7}-NPs or HEL₁₄₋₂₂/IA^{g7}-NPs (FIG. 9G) or with 2.5mi/IA^{g7}-NPs or IGRP₄₋₂₂/IA^{g7}-NPs (FIG. 12B). In FIG. 9G, IGRP₄₋₂₂/IA^{g7} and IGRP₁₂₈₋₁₄₅/IA^{g7} were tested in separate cohorts of mice. Mice treated with peptide or peptide-NPs (FIG. 9G) were randomized into either treatment within the same experiment. In vivo cytokine neutralization experiments involved administering mAbs against CD20 (5D2, a gift from A. Chan, Genentech; three doses of 250 μg i.v. on days 0-2 relative to the onset of hyperglycaemia) or 500 μg of HRPN (rIgG1), IFNγ (R4-6A2), IL-10 (JES5-2A5), TGF-β (1D11) or IL-21R (4A9) (BioXcell) i.p. twice a week for 2 weeks, followed by 200 μg per dose for 3 additional weeks. Mice were randomized into cytokine-blocking mAb-treatment (IFNγ, IL-10, TGFβ) or HRPN rat-IgG1 groups. Anti-CD20 and anti-IL21R mAbs were tested in separate cohorts of diabetic mice (FIG. 11A). Animals were assessed daily for glycosuria (corresponding to >16 Mm blood glucose) and given human insulin isophane (1 IU per day) s.c. if positive. Upon treatment withdrawal, NOD mice were monitored for recurrence of hyperglycaemia until 50 weeks of age.

Peptide, pMHC, pMHC-NP, Peptide-NP or Peptide-MP Therapy in EAE.

Six- to eight-week-old female C57BL/6, C57BL/6 Il27r^{-/-} or HLA-DR4-IE-transgenic C57BL/6 IAb^{null} mice were immunized with 150 μg of pMOG₃₅₋₅₅ or hMOG₉₇₋₁₀₈ or hPLP₁₇₅₋₁₉₂, respectively in CFA s.c. at the base of the tail, under isofluorane anaesthesia. The mice received 300 ng of Pertussis toxin i.v. on days 0 and 3. Mice were weighed and scored daily starting on day 10 after immunization. The score system used was been reported elsewhere30 and plotted over a 5-point scale. When most of the mice showed signs of advanced disease (day 14) or reached maximum disease scores (day 21), mice were divided into different treatment groups, synchronized for weight and disease score averages, and treated twice a week with pMHC-coated and uncoated nanoparticles, an identical amount of pMHC monomer, peptide-coated nanoparticles (at an equivalent dose of peptide), free peptide (8 μg per dose i.v. or s.c.) (Burton, B. R. et al. (2014) Nature Commun. 5:4741-4747), peptide-conjugated microparticles (15 μg of peptide per dose) (Getts, D. R. et al. (2012) Nature Biotechnol. 30:1217-1224) or unconjugated microparticles for 5 weeks. Mice were randomized into treatment with pMHC-NPs (one or two different types, depending on the experiment, as described in FIGS. 9J-9N, 10F-10H, 16A-16F, 17A-17I), uncoated nanoparticles or no treatment. Peptide, peptide-MPs, peptide-NPs, pMHC monomers and uncoated microparticles were tested together; mice were randomized into each treatment group as mice reached the indicated disease score (FIGS. 10F, 17F-17I). An additional control cohort was treated with a single dose of peptide-conjugated microparticles (FIG. 17H). Anti-cytokine and cytokine receptor mAb blocking studies (FIG. 18G) involved randomization of mice into each treatment group.

Peptide, pMHC-NP or Peptide-MP Therapy in CIA.

Bovine collagen II (bCII) dissolved in 0.05M acetic acid at 2 mg ml⁻¹ was emulsified in CFA (v/v) containing 4 mg ml⁻¹ of killed Mycobacterium tuberculosis (H37Ra). Eight- to twelve-week-old HLA-DR4-IE-transgenic C57BL/10.M mice were immunized intradermally at the base of the tail with 100 μg of bCII in CFA and boosted with 100 μg of bCII in IFA on days 14 and 28. The size of all four paws was measured using a caliper before immunization (day 0) and daily upon disease onset. Disease progression was measured as percentage increase in joint swelling relative to day 0. When this value reached 130%, mice were divided into different treatment groups and treated with pMHC-NPs, Cys-coated (pMHC unconjugated) NPs (25 μg of pMHC for pMHCNPs, or an equivalent amount of iron for Cys-conjugated NPs), free peptide (8 μg per dose s.c.) (Burton, B. R. et al. (2014) Nature Commun. 5:4741-4747) or peptide-conjugated MPs (15 μg of peptide per dose) (Getts, D. R. et al. (2012) Nature Biotechnol. 30:1217-1224) i.v. twice a week for 5 weeks. Mice were randomized into treatment with either pMHC-NP or uncoated nanoparticles, or into peptide or peptide-MP, respectively (FIG. 10A and FIG. 14-21). Mice were also assessed for clinical signs of disease up to a maximum clinical score of 12 as reported elsewhere (Leavenworth, J. W. et al. (2013) J. Clin. Invest. 123:1382-1389).

Peptide, pMHC-NP, Peptide-NP and Peptide-MP Therapy in Human PBMCreconstituted NSG Hosts.

PBMCs from new or recently diagnosed HLA-DRB1*0401⁺ or -DRB1*0301⁺ T1D patients (recruited with informed consent, approved by the Institutional Review Board at Hospital Clinic) were depleted of CD8⁺ T cells using anti-CD8 mAb-coated magnetic beads (Miltenyi Biotech) and injected i.v. (2×10⁷) into 8-10-week-old NSG hosts. Mice were treated with pMHC-NPs at the indicated doses, peptide-coated-NPs (at an equivalent dose of peptide), peptide alone (8 μg per dose s.c.) (Burton, B. R. et al. (2014) Nature Commun. 5:4741-4747) or peptide-conjugated microparticles (15 μg of peptide per dose) (Getts, D. R. et al. (2012) Nature Biotechnol. 30:1217-1224) starting on day 5 after PBMC transfusion, twice a week for 5 consecutive weeks, or left untreated. Individual patient samples were processed separately and injected into two (for pMHC-NP and peptide-NP experiments) or three separate mice (for peptide and peptide-MP experiments); one or two of the two-to-three hosts used in each of these experiments were treated and the other was left untreated (Tables 4A-4C). Therapy-induced expansion of cognate CD4⁺ T cells was measured in PLNs and/or spleen as described above. The HLA genotype, gender, age, months from diagnosis and type of pMHC-NP tested for each patient are summarized in Tables 4A-4C.

### Intraperitoneal Glucose Tolerance Tests.

Animals were fasted overnight and challenged with 2 mg kg-1 of d-glucose i.p. Blood glucose was monitored from the tail vein with a glucometer at different time points before and after glucose challenge. Serum insulin content was measured using the Mouse Ultrasensitive Insulin ELISA (ALPCO).

### Evaluation of Systemic Cellular and Humoral Immunity.

For the evaluation of cellular responses, pMHC-NP-treated and untreated female mice were injected with 2×10⁶ plaque-forming units (pfu) of recombinant Vaccinia Virus (rVV) i.v. Cohorts of mice were killed on day 4 and 14 after infection and processed for pMHC tetramer staining and rVV titre measurements. Briefly, the ovaries were weighed, homogenized using a pestle in 300 ul of RPMI-1640 containing 10% FBS, freezed-thawed 3 times followed by 3 rounds of sonication (20 seconds each). Serial dilutions of the lysates were added to confluent BSC-1 cell cultures in 6-well plates, incubated at 37° C. for 2 hours, washed twice with PBS and cultured in DMEM10. On day 2, the supernatants were discarded and the cell layers were stained with crystal violet to reveal plaques.

To evaluate humoral immunity, pMHC-NP-treated and untreated mice were immunized i.p. with 100 μg of DNP-KLH (Alpha Diagnostic International) in CFA. An identical boost was performed 3 weeks later. Mice were killed 10 days later. Anti-DNP antibody titres were measured by diluting serum samples in PBS containing 0.05% Tween 20. Anti-DNP antibodies were semi-quantified using an anti-DNP Ig ELISA Kit (Alphadiagnostic International) following the manufacturer's instructions.

### Proliferation and Cytokine Secretion Assays.

CD4⁺ T cells from pMHC-NP-treated mice were enriched from peripheral lymphoid organs using a BD Imag enrichment kit, stained with pMHC tetramers as described above and sorted by flow cytometry. For assays using memory and naive BDC2.5 CD4⁺ T cells, cells were enriched using Stem Cell Technologies enrichment kit, stained with antibodies and sorted. FACS-sorted cells (2-3×10⁴) were co-cultured with bone marrow-derived DCs (2×10⁴) pulsed with 2 μg ml⁻¹ of peptide. Supernatants were collected 48 hours later for measurement of cytokines via Luminex and the cells were pulsed with 1 microcurie (μCi) of (³H)-thymidine and collected after 24 hours to measure thymidine incorporation in triplicates.

To ascertain whether pMHC-NP therapy promoted the generation of IL-10-secreting B-cells in the PLNs of PBMC-engrafted NSG hosts, Applicant stained the PLN and splenic cell suspensions of individual mice with anti-hCD4-FITC, antihCD19-APC and tetramer-PE as described above, and sorted B-cells by flow cytometry (FACSAria-BD Biosciences). The B cells sorted from each organ were stimulated with LPS (1 μg ml⁻¹, Sigma) for 24 hours in RPMI-1640 supplemented with 10% human AB serum. The IL-10 content in the supernatants was measured in duplicates via Meso Scale technology using a V-PLEX Custom Human Cytokine kit for hIL-10 (Meso Scale Discovery). Data were normalized to the splenic B-cell values and reported as fold-change.

Isolation and In Vitro Stimulation of CD11b⁺ Cells from the PLNs and MLNs.

CD11b⁺ cells from LNs were obtained by digestion in collagenase D (1.25 μg ml⁻¹) and DNase I (0.1 μg ml⁻¹) for 15 min at 37° C. followed by purification with CD11b (BD Imag) mAb-coated magnetic beads. Cells were stimulated for 3 days with LPS (2 μg ml⁻¹) and the supernatants analysed for cytokine content with a Luminex multiplex cytokine assay.

### In Vitro Suppression Assays.

FACS-sorted 2.5mi/IA^{g7} tetramer positive or negative cells (2×10⁴) were co-cultured with bone marrow-derived DCs (2×10⁴) pulsed with 2 μg ml⁻¹ ‘suppressor’ (2.5mi or GPI₂₈₂₋₂₉₂) and ‘responder’ (gp33 or NRP-V7) peptides. Responder cells were CD8⁺ T cells (2×10⁴) purified from from 8.3-NOD or LCMV-Gp33-specific TCR-transgenic NOD mice using BD-Imag beads. These cells were labelled with CFSE (5 μM) and added to the DC cultures in duplicates or triplicates. Dilution of CSE in the responder cells was measured 48 hours later by FACS. In other experiments, the wells were supplemented within 24 hours of co-culture with HRPN rIgG, anti-IFNγ, anti-IL10 or anti-TGF-β (all 10 μg ml⁻¹) or the IDO inhibitor, 1-methyl tryptophan (1-MT; 400 μM).

### In Vivo Suppression of Crosspresentation.

For crosspresentation assays in non-transgenic mice, Applicant transfused CFSE-labelled 8.3-CD8⁺ reporter cells (5-10×10⁶) into untreated or pMHC-NP-treated mice and measured CFSE dilution in the hosts' lymphoid organs within 7 days after transfer.

### Adoptive Transfer of Suppression.

Splenic CD4⁺ or CD8⁺ T cells (107) from untreated mice or mice treated with 10 doses of 2.5mi/IA^{g7}-NPs or uncoated nanoparticles were transfused into 5-10 week-old NOD⁻ scid females. The hosts were transfused 24 hours later with 2×10⁷ CD4⁺ or CD8⁺ T-cell splenocyte mixtures purified from female NOD donors. The hosts were monitored for development of diabetes for at least 90 days after transfer (FIG. 9E). In another experiment, the hosts were treated twice a week with 2.5mi/IA^{g7}-NPs (FIG. 9E). In other experiments (FIG. 11E), CD4⁺ or CD8⁺ T-cell-reconstituted 5-6-week-old NOD scid females were transfused with 5×10⁵ CD19+ cells purified from the PLNs of mice treated with 10 doses of uncoated or 2.5mi/IA^{g7}-coated NPs during the preceding 5 week (FIG. 11D). B-cells were purified using the EasySep Mouse CD19-positive selection Kit II (StemCell Technologies). Other cohorts, studied separately (FIG. 11E), received PLN or MLN CD19+ cells (5×10⁵) plus total splenic CD4⁺ T cells (107) or 2.5mi/IA^{g7} tetramer⁺ (2×10⁵) or tetramer− CD4⁺ T cells (107) from 2.5mi/IA^{g7}-NP-treated donors. The hosts were randomized into each transfusion group and monitored for development of diabetes together. FIG. 11E includes data from the corresponding cohors studied in FIGS. 9E and 11D. Isolation of 2.5mi/IA^{g7} tetramer⁺ and tetramer− cells from total splenic CD4⁺ T cells of 2.5mi/IA^{g7}-NP-treated mice was performed using anti-PE mAb-coated microbeads and MACS LD columns (Miltenyi Biotec).

### B-Cell Proliferation and Breg Induction In Vivo and Breg Suppression In Vitro.

To isolate splenic DCs, spleens were digested in collagenase D and DNase for 15 minutes at 37° C. and DCs purified using anti-CD11c mAb-coated magnetic beads (MACS). The cells were pulsed with 10 μg ml⁻¹ of 2.5mi or GPI₂₈₂₋₂₉₂ peptide for 2 hours at 37° C. and labelled with CFSE (0.5 μM) or PKH26 (2 μM), respectively. Labelled cells (5-10×10⁶; mixed at 1:1 ratio) were administered i.v. into pMHC-NP-treated or untreated NOD mice. Three days later, Applicant compared the ratios of CFSE⁺ versus PKH26⁺ cells in the spleens of the different hosts by FACS. Similar experiments were done using peptide-pulsed splenic B cells isolated from female donor mice using anti-B220 mAb-coated magnetic beads (MACS).

For in vivo B_{reg} induction assays, B cells from NOD Il10^{GFP} (tiger) mice were enriched using a CD19 enrichment kit (Stem Cell Technologies) and pulsed with 2.5mi or GPI₂₈₂₋₂₉₂ peptides (10 μg ml⁻¹) for 2 hours at 37° C. The peptide-pulsed B cells were washed twice with PBS, labelled with PKH26 and transfused (1×10⁶) into pMHC-NP-treated or untreated mice. The hosts were killed 7 days later and their spleens labelled with anti-B220-APC and biotinylated anti-CD1d or anti-CD5 mAbs and Streptavidin-PerCP. PKH26⁺ cells were analysed for presence of eGFP⁺CD1d^{high} or CD5+ cells by flow cytometry.

To determine the role of T_{R}1-derived cytokines in B_{reg} formation (FIG. 18U), Applicant repeated the experiments described above but using 3×10⁶ B cells and hosts treated with 250 or 500 μg (given i.p. daily from day—3 to day 6 relative to B-cell transfer) of anti-HRPN (rIgG1), anti-IL-10 (JES5-2A5), anti-TGFβ (1D11) or anti-IL-21R (4A9) mAbs (BioXcell). Hosts were randomized into each antibody-treatment group and studied together.

To measure the ability of the T_{R}1-induced B_{reg} cells to suppress the antigen-induced activation of T cells in vitro, Applicant isolated CD19⁺ B cells from the PLNs of age-matched untreated NOD mice or NOD mice treated with 10 doses of 2.5mi/IA^{g7}-NPs and cultured these cells with LPS (10 μg ml⁻¹) overnight. Applicant then cultured these cells (2×10⁴) with 2.5mi-peptide-pulsed (0.1 μg ml⁻¹) bone marrow-derived DCs (2×10⁴) and CFSE-labelled BDC2.5 CD4+ cells (4×10⁴). Dilution of CFSE in CD4⁺ cells was measured 3 days later.

Cd25⁺Cd4⁺ T_{Reg} Depletion.

NOD mice were treated with 500 μg of anti-CD25 (PC61.5.3, BioXcell) i.p. 3 times weekly from 8 weeks of age, followed by 10-injections of pMHC-NPs given twice weekly starting at 10 weeks of age. Average CD4⁺CD25⁺ and FOXP3-eGFP⁺CD4⁺ T-cell depletion was 90% and 70%, respectively.

### Histology.

Tissues were fixed in 10% formalin and embedded in paraffin. H&Estained pancreata were scored for insulitis as reported (Verdaguer, J. et al. (1997) J. Exp. Med. 186:1663-1676). Briefly, insulitis was scored as: 0, none; 1, peri-insulitis; 2, infiltration covering <25% of the islet; 3, covering 25-50% of the islet; and 4, covering >50% of the islet.

Spinal cord and brain tissues were fixed in 10% buffered formalin for a minimum of 24 hours, embedded in paraffin and sectioned at 6 μm. Slides from paraffinembedded tissues were deparaffinized and subjected to antigen retrieval by steaming the slides in 10 mM sodium citrate buffer (pH 6.0) for 20 min and cooling at room temperature for 20 min. For immunohistochemistry, slides were fixed with 10% formalin and treated with 3% H₂O₂ in methanol at −20° C. Sections were permeabilized with 0.25% Triton-X 100 and blocked with a skim milk blocking solution. Rabbit anti-IBA1 (Wako, 1:500) or rat anti-MBP (Abcam) were incubated at 4° C. overnight followed by respective biotinylated secondary antibodies (1:500), avidin-biotin complex, and 3,3′-diaminobenzidine. Sections were counterstained with haematoxylin and eosin, dehydrated with graded ethanol and mounted with Acrytol. For histological myelin staining, slides were fixed with 10% formalin or deparaffinized, dehydrated with graded ethanol, and incubated with 0.2% luxol fast blue in 95% ethanol at 65° C. Slides were developed in 0.05% lithium carbonate, counterstained with haematoxylin and eosin, and mounted with Acrytol. Images of cerebellum were taken on an Olympus bright-field microscope. Inflammatory foci (dense nuclear clusters or perivascular cuffs with corresponding demyelination) were counted and their size measured using ImageJ software. For quantification of relative IBA1 intensity, blinded observers ranked images from highest to lowest intensity.

Knee joints from bCII-immunized mice were fixed in 4% buffered formalin overnight, and decalcified with 14% EDTA over 3 weeks. Decalcified paws were embedded in paraffin, sectioned at 8 μm and stained with haematoxylin and eosin to score infiltration and pannus formation on a scale of 5, where 5 corresponds to erosive arthritis, with severe infiltration and pannus covering 60% of the joint space. Proteoglycan depletion at the articular surface of the tibia and femur was assessed by the loss of safranin-O stain intensity. For this, sections were deparaffinized, hydrated and stained with haematoxylin before staining with 0.05% aqueous fast green for 5 min. Slides were fixed with 1% acetic acid and stained with 0.1% aqueous safranin-O for 2 min, dehydrated with graded ethanol, cleared with xylene and mounted with DPX. Scoring was done on a scale of 0 to 3 corresponding to: 0, 0% depletion, 1, low (<25%), 2, moderate (25-50%), and 3, severe (>50%). Destruction of articular cartilage included an assessment of the presence of dead chondrocytes (empty lacunae) and was scored on a scale of 3 (0, no empty lacunae; 3, complete loss of chondrocytes on articular cartilage/severe cartilage erosion).

### Isolation of CNS-Infiltrating Lymphocytes.

Mice were anesthetized with Ketamine-Xylazine and perfused with PBS through the heart left ventricle. The brain and spinal cord were isolated manually, cut into small fragments and digested with a solution of collagenase D (1.25 μg ml⁻¹) and DNase I (1% w/v) in HBSS for 30 min at 37° C. The digested CNS was passed through a 70 μm cell strainer. Cells were resuspended in DMEM (supplemented with 2% FBS and 10 mM HEPES) and 100% Percoll (to a final Percoll concentration of 30%). The solution was layered onto 65% Percoll and centrifuged at 380g for 30 min at room temperature. The mononuclear cell layer lying at the interphase was washed with RPMI before further analyses.

### Quantitative RT-PCR.

RNA was extracted from 2.5mi/IA^{g7} tetramer⁺ or tetramer⁻ CD4⁺ T cells sorted from 2.5mi/IA^{g7}-NP-treated NOD mice and stimulated in vitro with anti-CD3/anti-CD28 mAb-coated dynabeads.

Each tetramer⁺ sample corresponded to cells pooled from 2-3 mice. RNA was reverse transcribed and cDNA plated in Mouse Immunology 384 StellArray qPCR plates (Bar Harbour BioTechnology) with 2×SYBR Green Master Mix (Applied Biosystems). The plate was run in a 7900HT Applied Biosystems realtime PCR instrument, and the raw data was analysed using the Global Pattern Recognition (GPR) analysis tool (http://www.gene-quantification.com/qpcr-array.html). mRNA isolated from additional samples was subjected to RT-qPCR using primers specific for IL-21 (Forward: 5′-TCATCATTGACCTCGTGGCCC-3′; Reverse: 5′-ATCGTACTTCTCCACTTGCAATCC-3′), IL-10 (Forward: 5′-CTTGCACTACCAAAGCCACA-3′; Reverse: 5′-GTTATTGTCTTCCCGGCTGT-3′), c-Maf (Forward: 5′-AGCAGTTGGTGACCATGTCG-3′; Reverse: 5′-TGGA GATCTCCTGCTTGAGG-3′), IFN-γ (Forward: 5′-TGAACGCTACACACTGCA TCTTGG-3′; Reverse: 5′-CGACTCCTTTTCCGCTTCCTGAG-3′), LAG-3 (Forward: 5′-TCCCAAATCCTTCGGGTTAC-3′; Reverse: 5′-GAGCTAGACTCTGCGGCGTA-3′), CD49b (Forward: 5′-CCGGGTGCTACAAAAGTCAT-3′; Reverse: 5′-GTCGGCCACATTGAAAAAGT-3′), Aryl Hydrocarbon Receptor (Forward: 5′-CGTCCCTGCATCCCACTACTT-3′; Reverse: 5′-GGACATGGCCCCAGCATAG-3′) and ICOS (Forward: 5′-TGACCCACCTCCTTTTCAAG-3′; Reverse: 5′-TTAGGGTCATGCACACTGGA-3′).

pMHC-NP-induced upregulation of T_{R}1 transcripts in in vitro-activated CD4⁺ T cells was performed by culturing mouse naive eGFP-BDC2.5-CD4⁺ T cells from BDC2.5 NOD Foxp3-eGFP mice (CD62L^{hi}FOXP3⁻eGFP⁻; 1.5×10⁶ ml⁻¹) with anti-CD3/anti-CD28 mAb-coated microparticles (1 bead per cell) for three days in the absence of APCs, followed by a one day culture of re-purified (micro particle-free) CD4⁺ T cells in rhIL-2 (30 IU ml-1), and a 6-day culture with 2.5mi peptide (10 μg ml⁻¹), 2.5mi/IA^{g7} monomers (25 μg pMHC per ml), 2.5mi/IA^{g7}-NPs (25 μg pMHC per ml and 50 μg ml⁻¹ iron), or unconjugated nanoparticles (50 μg iron per ml). Relative gene expression was calculated using unstimulated cultures as controls.

pMHC-NP-induced upregulation of T_{R}1 transcripts in naive compared to memory BDC2.5 CD4⁺ T cells in vivo was done by transfusing naive (CD44^{med}CD62L^{hi}) or memory (CD44^{hi}CD62L^{low}) eGFP-CD4⁺ T cells from BDC2.5-TCR-transgenic NOD or NOD Foxp3-eGFP mice (Thy1^{b+}) (1-1.5×10⁶ cells per host) into NOD.Thy1^{a} hosts and by treating the hosts with four doses of 2.5mi/IA^{g7}-NPs over two weeks or leaving them untreated. Two and a half weeks later, Thy1^{b+}CD4⁺ T cells were sorted from the hosts and challenged with anti-CD3 and anti-CD28-coated magnetic Dynabeads for 3 days before mRNA extraction and RT-qPCR using primers specific for c-Maf, IL-21, IL-10, IFNγ, LAG-3 and CD49b.

To compare levels of IL-10 mRNA in the tetramer⁺ compared with tetramer CD4⁺ T cells of pMHC-NP-treated PBMC-engrafted NSG hosts, Applicant stained splenocytes with anti-hCD4-FITC, anti-hCD19-APC and tetramer-PE as described above, and sorted tetramer+ and tetramer− cells from individual hosts by FACS (FACSAria-BD Biosciences). Sorted cells were cultured for 72 h in RPMI-1640 containing 10% human AB serum, in the presence of Dynabeads Human T-Activator CD3/CD28 (LifeTechnologies) using a 1:1 cell to bead ratio. Total RNA from cell pellets was reverse-transcribed using a dual reverse transcriptase/lysis solution containing 5 mM DTT, 2 U ml⁻¹ RNAase, 500 mM dNTPs, 10 U ml⁻¹ of Superscript reverse transcriptase (Invitrogen, LifeTechnologies), 100 mg ml⁻¹ bovine serum albumin, 1% Triton X-100, 25 ng ml⁻¹ Oligo dT (Invitrogen), 0.5 nM spermidine, and 1× First Strant buffer (Invitrogen) in 20 μl for 60 min at 50° C. and 15 min at 70° C. We then mixed 1 μl of the cDNA reaction volume with 12.5 μl of Power SyBRGreen PCR master mix solution (Applied Biosystem) and amplified with a real-time PCR machine (7900HT, Applied Biosystems) using the following primers: β-actin (Forward: 5′-CTGGAACGGTGAAGGTGACA-3′; Reverse: 5′-AAGGGACTTCCTGTAACAATGCA-3′), IL-10 (Forward: 5′-AA GACCCAGACATCAAGGCG-3′; Reverse: 5′-AATCGATGACAGCGCCGT AG-3′).

### Statistical Analyses.

The sample size values described in the figure legends correspond to the number of individual mice tested (not replicates) and data shown correspond to pooled data from different experiments. Data were compared by Student's t-test, Mann-Whitney U-test, chi-square, log-rank (Mantel-Cox), Pearson correlation or two-way ANOVA tests. Statistical significance was assumed at P<0.05.

### Example 3. pMHC Valency and Density Effects In Vivo

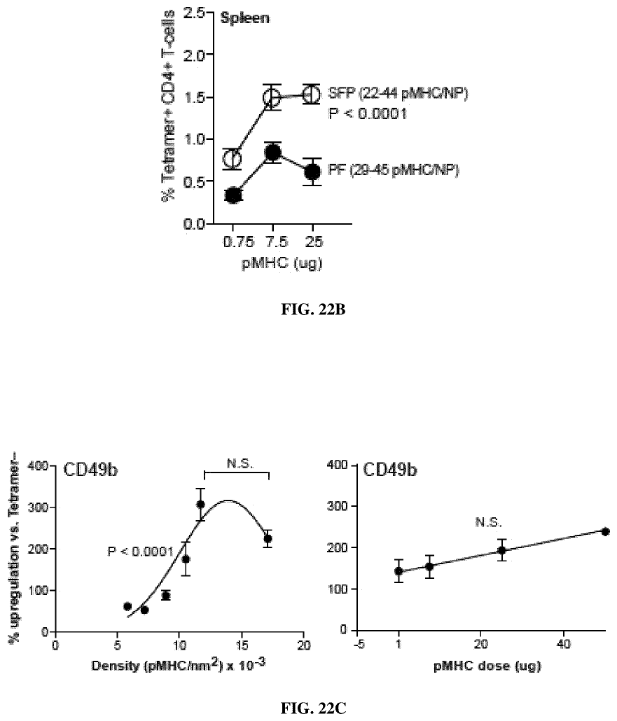

Applicant next tested the predictions of the mathematical model experimentally, by comparing the Treg cell expanding properties of various preparations of PF-M (˜20 nm) and SFP-Z (˜8 nm) NPs coated with 2.5mi/IA^{g7} pMHCs, which expand cognate T-regulatory-1 (T_{R}1) type CD4⁺ T-cells. Comparison of the Treg expanding properties of 7 different 2.5mi-IA^{g7}-PF-M preparations, carrying from 29-59 pMHCs/NP demonstrated clear pMHC dose-dependent effects within individual preparations, but also no significant effects of pMHC valency across batches (FIG. 22A). Importantly, however, studies using the smaller 2.5mi-IA^{g7}-SFP-Z preparations carrying 22-44 pMHCs/NP indicated significantly higher Treg expanding effects, at all doses tested (0.75-1 μg, 7.5-10 μg and 25 μg of total pMHC/dose), than 2.5mi-IA^{g7}-PF-M particles carrying 29-45 pMHCs/NP (FIG. 22B). These results were further confirmed by producing 11 nm diameter PF-M NPs and testing the ability of their 2.5mi-IA^{g7}-coated counterparts to expand cognate T_{R}1 T-cells in vivo. Remarkably, 11 nm PF-M NPs delivering 7.5 μg of total pMHC at 15 pMHCs/NP expanded cognate T_{R}1 cells to 1.6±0.3% of total splenic CD4+ T-cells, a value comparable to the SFP series of NPs delivering 7.5 μg of 22-44 pMHCs/NP.

As noted above, Applicants have shown that autoreactive memory CD4⁺ T-cells express a T-regulatory type 1 (T_{R}1)-poised transcriptional program and export LAG3 but not CD49b (T_{R}1 markers) to the cell surface. Since 2.5mi-IA^{g7}-NP therapy triggers the expression of IL-10 and the upregulation of CD49b on T_{R}1-poised memory T-cells, hence promoting their conversion into stable T_{R}1 cells, Applicant questioned if the efficiency of these processes was also regulated by pMHC density on the NP surface. Remarkably, there was a statistically significant correlation between 2.5mi/IA^{g7} density (but not total pMHC dose) and CD49b (but not LAG3) upregulation on the T_{R}1-like CD4+ T-cells that expand in wild-type NOD mice in response to 2.5mi/IA^{g7}-NP therapy; this effect peaked at ˜0.012 pMHC/nm² (FIG. 22C). Together, these results support the idea that pMHC density is a critical parameter in the design of pMHC-based nanomedicines.

These effects of pMHC density on biological activity were also seen in vivo; increases in pMHC density led to enhanced upregulation of the T_{R}1 cell marker CD49b in pMHC-NP-treated mice, suggesting that pMHC density is responsible for promoting Treg fitness. Whereas total pMHC dose was associated with the Treg-expanding properties of these nanomedicines, it only had minor effects on this phenotype, suggesting that pMHC density and pMHC dose have separate roles in promoting Treg conversion and expansion, respectively.

### EQUIVALENTS

It should be understood that although the present disclosure has been specifically disclosed by certain embodiments and optional features, modification, improvement and variation of the disclosures embodied disclosed herein may be resorted to by those skilled in the art, and that such modifications, improvements and variations are considered to be within the scope of this disclosure. The materials, methods, and examples provided here are representative of certain embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Throughout this disclosure, various publications, patents and published patent specifications are referenced by an identifying citation. All publications, patent applications, patents, and other references mentioned herein are expressly incorporated by reference in their entirety, to the same extent as if each were incorporated by reference individually. In case of conflict, the present specification, including definitions, will control.

TABLE 1
Functionalized PEG linkers

| Linker | Types of | | M.W. | Functional | |
|---|---|---|---|---|---|
| Code | Nanoparticle | PEG Linkers | (kDa) | group | Structure |
| A1 | Gold nanoparticle (GNP-C) | Thiol-PEG- carboxyl | 3.5 | Amine (—NH₂) | 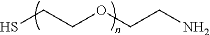 |
| A2 | Gold nanoparticle (GNP-N) | Thiol-PEG- amine | 3.5 | Carboxyl (—COOH) | 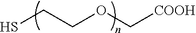 |
| S1 | Iron oxide Nanoparticle (SFP-C) | Dopamine- PEG- carboxyl | 3.5 | Carboxyl (—COOH) | 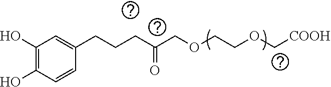 |
| S2 | Iron oxide Nanoparticle (SFP-N) | Dopamine- PEG- amine | 3.5 | Amine (—NH₂) | 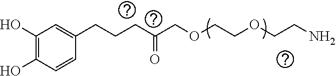 |
| S3 | Iron oxide Nanoparticle (SFP-Z) | Dopamine- PEG- azide | 3.5 | Azide (—N₃) | 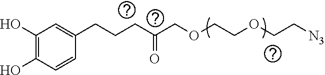 |
| S4 | Iron oxide Nanoparticle (SFP-M) | Dopamine- PEG- maleimide | 3.5 | 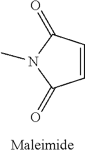 | 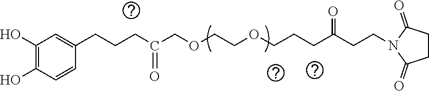 |
| S5 | Iron oxide Nanoparticle (SFP-O) | Dopamine- PEG- Orthopyridyl disulfide | 3.5 | 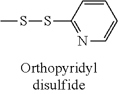 | 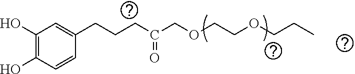 |
| P1 | Iron oxide Nanoparticle (PF-C) | carboxyl- PEG- carboxyl | 2.0 | Carboxyl (—COOH) | 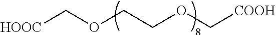 |
| P2 | Iron oxide Nanoparticle (PF-N) | Methoxy- PEG- amine | 2.0 | Amine (—NH₂) | 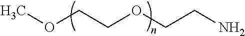 |
| P3 | Iron oxide Nanoparticle (PF-M) | Methoxy- PEG- maleimide | 2.0 | 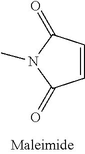 | 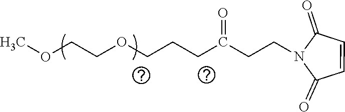 |
| P4 | Iron oxide Nanoparticle (PF-O) | Methoxy- PEG- Orthopyridyl disulfide | 2.0 | 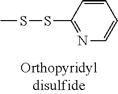 | 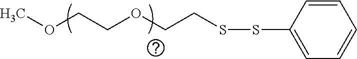 |
| P5 | Iron oxide Nanoparticle (PF) | PEG | 2.0 | Hydroxyl (—OH) | 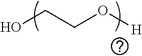 |

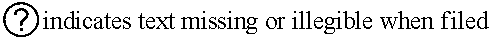

TABLE 2
Codon Table

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACI |
| Valine | Val | V | GUA GUC GUG GUU |
| Tryptophan | Trp | W | UGG |
| Tyrosine | Tyr | Y | UAC UAU |

TABLE 3A
Real-time RT-PCR for 384 immunological marker's

| Gene | | | GPR | GPR Fold |
|---|---|---|---|---|
| Name | Protein | Function | P-value | Change |
| Spp1 | Osteopontin | ECM protein component | 0.001285 | 123.4154 |
| Il10 | IL-10 | Immunosuppressive cytokine | 0.000677 | 41.61113 |
| Gzma | Granzyme A | Cytolytic enzyme | 0.008105 | 41.933044 |
| Lepr | Leptin R | Regulator of survival and activation | 0.049202 | 17.752507 |
| | | of T-cells. | | |
| Ido1 | Indoleamine | Key enzyme of the tryptophan | 0.036635 | 14.462784 |
| | 2,3-dioxygenase | catabolism. Inhibitory enzyme. | | |
| | (IDO1) | | | |
| Il13 | IL-13 | Th2 and anti-inflammatory | 0.021696 | 11.702363 |
| | | cytokine | | |
| Entpd1 | Ectonucleotide | ATP/ADP hydrolase Memory | 0.006004 | 8.192557 |
| | triphosphate | marker and inhibitory enzyme | | |
| | diphosphate 1 | | | |
| | (CD39) | | | |
| Prdm1 | Blimp-1 | Transcription factor (T_{R}1 cells) | 0.006751 | 8.826929 |
| Tbx21 | T-bet | Th1 Transcription factor | 0.01586 | 7.829291 |
| Pdcd1 | PD-1 | Inhibitory receptor | 0.022 | 6.5673 |
| Il9 | IL-9 | Th2 cytokine | 0.039733 | 6.322661 |
| Il21 | IL-21 | Th1 and T_{R}1 cytokine | 0.019428 | 6.169626 |
| Csf1 | M-CSF | Macrophage growth factor | 0.01446 | 5.397609 |
| Il1r2 | IL-1R2 | IL-1 Decoy receptor. | 0.038197 | 3.470605 |
| | | Inhibits IL-1 signaling | | |
| Casp1 | Caspase-1 | Pro-inflammatory zymogen that | 0.037868 | 2.966331 |
| | | cleaves IL-1β and IL-18 | | |
| Cx3cr1 | CX3CR1 | CX3CL1 (fractalkine) receptor; | 0.004775 | −139.568489 |
| | | T-cell chemokine | | |
| Foxp3 | Foxp3 | nTreg-specific transcription factor | 0.047705 | −57.878553 |
| Cxcl9 | CXCL9 | Th1 T-cell chemokine | 0.00246 | −54.48584 |
| Il18r1 | IL-18R | IL-18 (Th1 amplifying factor) | 0.011622 | −7.293964 |
| Sell | CD62L | Selectin present in naïve T-cells | 0.012487 | −6.23221 |
| Ccr7 | CCR7 | Thymus and LN-driving chemokine | 0.027056 | −3.383342 |
| | | Naïve/memory marker | | |
| Tfrc | Transferrin | Iron-binding regulator of iron | 0.035692 | −2.928147 |
| | | homeostatis | | |

TABLE 3B
Real-time RT-PCR for T_{R}1 transcripts

| Gene | | | | Fold |
|---|---|---|---|---|
| Name | Protein | Function | P-value | Change |
| Il21 | IL-21 | Th1 and T_{R}1 cytokine | 0.0061 | 104.15 |
| Il10 | IL-10 | Immunosuppressive cytokine | 0.0061 | 79.65 |
| c-Maf | c-MAF | IL-10-regulating transcription | 0.0061 | 18.455 |
| | | factor | | |
| Ifng | IFNγ | Th1 cytokine | 0.0061 | 11.2 |
| Lag-3 | Lag-3 | Inhibitory receptor. | 0.0061 | 5.471 |
| | | T_{R}1 marker | | |
| Itga2 | CD49b | Integrin | 0.1091 | 5.054 |
| | | T_{R}1 marker | | |
| Ahr | Aryl | T_{R}1-inducer receptor | 0.0727 | 3.138 |
| | hydrocarbon | | | |
| | receptor | | | |
| Icos | ICOS | Costimulatory molecule | 0.0424 | 3.033 |

TABLE 4A
DRB1*0401+ patients/GAD65_{555-567 (557I)}/DR4- or PPI_{76-90 (88S)}/DR4-NP

| | | | | | | | | | | Tetramer | |
|---|---|---|---|---|---|---|---|---|---|---|---|
| | | | Age at | | | | | hCD4 | Tetramer | control | |
| | | Age | onset | Anti- | Anti- | Anti- | pMHC-N | (% of | (% of | (% of | Out- |
| Code | Gender | (yr) | (yr) | GAD | IA2 | INS | (10 ug/dose) | MNCs) | hCD4)* | hCD4) | come |
| 8007 | M | 35 | 34 | + | + | + | GAD65_{555-567 (557I)}/DR4 | 80 | 0.098 | 0.170 | − |
| 8014 | F | 44 | 43 | + | − | ND | GAD65_{555-567 (557I)}/DR4 | 52.5 | 1.310 | 0.312 | + |
| 7005 | F | 52 | 50 | + | + | ND | GAD65_{555-567 (557I)}/DR4 | 55.2 | 0.127 | 0.418 | − |
| 8015 | F | 41 | 40 | ND | ND | ND | GAD65_{555-567 (557I)}/DR4 | 67.4 | 0.087 | 0.128 | − |
| 7005 | F | 52 | 50 | + | + | ND | PPI_{76-90 (88S)}/DR4 | 65.5 | 3.080 | 0.062 | + |
| 8015 | F | 41 | 40 | ND | ND | ND | PPI_{76-90 (88S)}/DR4 | 83.0 | 0.125 | 0.051 | − |
| Mean | | 44.2 | 42.8 | | | | | 67.3 | 0.805 | 0.190 | |
| (SE) | | (2.8) | (2.56) | | | | | (5.1) | (0.496) | (0.060) | |
| Median | | 42.5 | 41.5 | | | | | | | | |
| (range) | | (35-52) | (34-50) | | | | | | | | |

*Result was considered (+) if greater than the mean + 3 S.E. from the samples stained with control tetramer

TABLE 4B
DRB1*0301+ patients/IGRP₁₃₋₂₅-DR3-NP

| | | | | | | | Spleen | | LN | | |
|---|---|---|---|---|---|---|---|---|---|---|---|
| | | | | | | | (% of hCD4) | | (% of hCD4) | | |
| | | | Age at | | | | Treated | | Treated | | |
| | | Age | onset | Anti- | Anti- | Anti- | (20 ug/ | Un- | (20 ug/ | Un- | Out- |
| Code | Gender | (yr) | (yr) | GAD | IA2 | INS | dose)* | treated | dose)* | treated | come |
| 8030 | M | 27 | 27 | + | + | ND | 0.859 | 0.168 | 3.170 | 0.198 | + |
| 8025 | M | 46 | 46 | + | + | ND | 0.376 | 0.167 | 0.174 | 0.000 | + |
| 8033 | M | 39 | 39 | + | − | ND | 0.495 | 0.310 | 0.034 | 0.048 | + |
| 8020 | F | 20 | 19 | + | + | − | 0.641 | dead | no cells | dead | + |
| 8028 | F | 22 | 21 | + | + | − | 0.044 | 0.086 | 0.627 | 0.101 | + |
| 8016 | M | 45 | 45 | + | − | ND | 0.539 | sick | 1.270 | sick | + |
| 8038 | F | 31 | 31 | + | − | ND | 0.130 | 0.163 | 2.410 | no cells | + |
| Mean (SE) | | 32.9 | 32.6 | | | | 0.441 | 0.224 | 1.281 | 0.087 | |
| | | (4.0) | (4.2) | | | | (0.108) | (0.036) | (0.518) | (0.037) | |
| Median (range) | | 31 | 31 | | | | | | | | |
| | | (20-46) | (19-46) | | | | | | | | |
| P vs. DR4 patients | 0.135 | 0.02 | 0.032 | | | | | | | | |
| P treated vs | | | | | | | 0.027 | | 0.035 | | |
| untreated | | | | | | | (%) | | (%) | | |
| | | | | | | | 0.042 | | 0.028 | | |
| | | | | | | | (abs #) | | (abs #) | | |
| % in hCD4 in | | | | | | | 44.7 | 49.6 | 54.7 | 45.6 | |
| MNC − Mean | | | | | | | (6.2) | (3.2) | (8.6) | (10.9 | |
| (SE) | | | | | | | | | | | |

*Result was considered (+) if greater than the mean + 3 S.E. corresponding to the samples from PBMC-reconstituted but pMHC-NP-untreated NSG hosts.
** The PLN samples with increased IGRP 13-25/DR3 tetramer+ cells were significantly enlarged and had increased cellularity as compared to those from treated mice lacking such increases (P = 0.048) or from untreated mice (P = 0.036). There was a statistically significant correlation between % tetramer + cells and total PLN cell number (r2 = 0.455; P = 0.032)

TABLE 4C
DRB1*0301+ patients (IGRP₁₃₋₂₅ peptide, IGRP₁₃₋₂₅-peptide-NP and IGRP₁₃₋₂₅ peptide-MP)

| | | | Age at | | | | Spleen (% of hCD4) | | | |
|---|---|---|---|---|---|---|---|---|---|---|
| | | Age | onset | Anti- | Anti- | Anti- | Peptide | MC | Peptide- | Un- |
| Code | Gender | (yr) | (yr) | GAD | IA2 | INS | treated | treated | NP | treated |
| 8049 | M | 28 | 25 | − | + | ND | 0.026 | 0.023 | | dead |
| 8035 | M | 30 | 28 | + | − | − | 0.121 | 0.073 | | 0.050 |
| 8040 | M | 24 | 23 | + | + | − | 0.145 | 0.085 | | 0.161 |
| 8047 | F | 25 | 25 | + | + | − | 0.116 | 0.127 | | 0.093 |
| 8042 | M | 33 | 31 | + | − | ND | 0.010 | 0.005 | | dead |
| 8050 | M | 23 | 23 | − | − | − | | | 0.041 | dead |
| 8020 | F | 21 | 20 | + | + | − | | | 0.089 | 0.051 |
| 7010 | M | 21 | 17 | ND | ND | ND | | | 0.109 | dead |
| 5023 | M | 12 | 12 | ND | ND | ND | | | 0.143 | dead |
| Mean | | 24.1 | 22.7 | | | | 0.084 | 0.063 | 0.096 | 0.089 |
| (SE) | | (2.0) | (1.9) | | | | (0.027) | (0.022) | (0.021) | (0.026) |
| Median | | 24.0 | 23.0 | | | | | | | |
| (range) | | (12-33) | (12-31) | | | | | | | |
| p** | | | | | | | 0.448 | 0.236 | 0.424 | |
| treated vs | | | | | | | (%) | (%) | (%) | |
| untreated | | | | | | | 0.227 | 0.437 | 0.231 | |
| | | | | | | | (abs #) | (abs #) | (abs #) | |
| % hCD4 | | | | | | | 56.9 | 43.8 | 47.6 | 39.9 |
| in MNC − | | | | | | | (2.9) | (4.2) | (3.8) | (5.7) |
| Mean | | | | | | | | | | |
| (SE) | | | | | | | | | | |

| | PLN (% of hCD4) | | | | |
|---|---|---|---|---|---|
| | Peptide | MC | Peptide- | Un- | Out- |
| Code | treated | treated | NP | treated | come |
| 8049 | No | No | | dead | − |
| | cells | cells | | | |
| 8035 | No | No | | No | − |
| | cells | cells | | cells | |
| 8040 | No | No | | No | − |
| | cells | cells | | cells | |
| 8047 | No | No | | No | − |
| | cells | cells | | cells | |
| 8042 | No | 0.002 | | dead | − |
| | cells | | | | |
| 8050 | | | 0.049 | dead | − |
| 8020 | | | No | No | − |
| | | | cells | cells | |
| 7010 | | | 0.020 | dead | − |
| 5023 | | | No | dead | − |
| | | | cells | | |
| Mean | | 0.002 | 0.335 | | |
| (SE) | | (0.000) | (0.015) | | |
| Median | | | | | |
| (range) | | | | | |
| p** | | | | | |
| treated vs | | | | | |
| untreated | | | | | |
| % hCD4 | | 43.2 | 43.3 | | |
| in MNC − | | (0.0) | (3.0) | | |
| Mean | | | | | |
| (SE) | | | | | |

* Result was considered (+) if greater than the mean + S.E. corresponding to the samples from PBMC-reconstituted but untreated NSG hosts.

### REFERENCES

1 Lieberman, S. & DiLorenzo, T. A comprehensive guide to antibody and T-cell responses in type 1 diabetes. Tissue Antigens 62, 359-377 (2003).
2 Tsai, S., Shameli, A. & Santamaria, P. CD8⁺ T-cells in autoimmune diabetes. Adv. Immunol. 100, 79-124 (2008).
3 Santamaria, P. The long and winding road to understanding and conquering type 1 diabetes. Immunity 32, 437-445 (2010).
4 Babbe, H. et al. Clonal expansions of CD8(+) T cells dominate the T cell infiltrate in active multiple sclerosis lesions as shown by micromanipulation and single cell polymerase chain reaction. J. Exp. Med. 192, 393-404 (2000).
5 Zang, Y. C. et al. Increased CD8+ cytotoxic T cell responses to myelin basic protein in multiple sclerosis. J. Immunol. 172, 5120-5127 (2004).
6 Walter, U. & Santamaria, P. CD8+ T cells in autoimmunity. Curr. Opin. Immunol. 17, 624-631 (2005).
7 Firestein, G. S. Evolving concepts of rheumatoid arthritis. Nature 423, 356-361 (2003).
8 Sakaguchi, S. et al. Foxp3+ CD25+ CD4+ natural regulatory T cells in dominant self-tolerance and autoimmune disease. Immunol. Rev. 212, 8-27 (2006).
9 Miyara, M. et al. Functional delineation and differentiation dynamics of human CD4+ T cells expressing the FoxP3 transcription factor. Immunity 30, 899-911 (2009). Chen, Q., Kim, Y. C., Laurence, A., Punkosdy, G. A. & Shevach, E. M. IL-2 controls the stability of Foxp3 expression in TGF-beta-induced Foxp3+ T cells in vivo. J. Immunol. 186, 6329-6337 (2011).
11 Yang, X. P. et al. Opposing regulation of the locus encoding IL-17 through direct, reciprocal actions of STAT3 and STAT5. Nat. Immunol. 12, 247-25 (2011).
12 Marwaha, A. K. et al. Cutting edge: Increased IL-17-secreting T cells in children with new-onset type 1 diabetes. J. Immunol. 185, 3814-3818 (2010).
13 McClymont, S. A. et al. Plasticity of human regulatory T cells in healthy subjects and patients with type 1 diabetes. J. Immunol. 186, 3918-3926 (2011).
14 Komatsu, N. et al. Heterogeneity of natural Foxp3+ T cells: a committed regulatory T-cell lineage and an uncommitted minor population retaining plasticity. Proc. Natl. Acad. Sci. U.S.A. 106, 1903-1908 (2009).
15 Zhou, L., Chong, M. M. & Littman, D. R. Plasticity of CD4+ T cell lineage differentiation. Immunity 30, 646-655 (2009).
16 Zhou, X. et al. Instability of the transcription factor Foxp3 leads to the generation of pathogenic memory T cells in vivo. Nat. Immunol. 10, 1000-1007 (2009).
17 Komatsu, N. et al. Pathogenic conversion of Foxp3+ T cells into TH17 cells in autoimmune arthritis. Nat. Med. 20, 62-68 (2014).
18 Bailey-Bucktrout, S. L. et al. Self-antigen-driven activation induces instability of regulatory T cells during an inflammatory autoimmune response. Immunity 39, 949-962 (2013).
19 Bacchetta, R. et al. High levels of interleukin 10 production in vivo are associated with tolerance in SCID patients transplanted with HLA mismatched hematopoietic stem cells. J. Exp. Med. 179, 493-502 (1994).
20 Roncarolo, M. G. et al. Interleukin-10-secreting type 1 regulatory T cells in rodents and humans. Immunol. Rev. 212, 28-50 (2006).
21 Gagliani, N. et al. Coexpression of CD49b and LAG-3 identifies human and mouse T regulatory type 1 cells. Nat. Med. 19, 739-746 (2013).
22 McLarnon, A. IBD: Regulatory T-cell therapy is a safe and well-tolerated potential approach for treating refractory Crohn's disease. Nature Rev Gastroenterol Hepatol 9, 559 (2012).
23 Desreumaux, P. et al. Safety and Efficacy of Antigen-Specific Regulatory T-Cell Therapy for Patients With Refractory Crohn's Disease. Gastroenterology 143, 1207-1217 (2012).
24 Roncarolo, M. G., Gregori, S., Lucarelli, B., Ciceri, F. & Bacchetta, R. Clinical tolerance in allogeneic hematopoietic stem cell transplantation. Immunol. Rev. 241, 145-163 (2011).
25 Tsai, S. et al. Reversal of autoimmunity by boosting memory-like autoregulatory T cells. Immunity 32, 568-580 (2010).
26 Clemente-Casares, X., Tsai, S., Yang, Y. & Santamaria, P. Peptide-MHC-based nanovaccines for the treatment of autoimmunity: a “one size fits all” approach? J. Mol. Med. 89, 733-742 (2011).
27 Stratmann, T. et al. Susceptible MHC alleles, not background genes, select an autoimmune T cell reactivity. J. Clin. Invest. 112, 902-914 (2003).
28 Mukherjee, R., Wagar, D., Stephens, T., Le-Chan, E. & Singh, B. Identification of CD4+ T cell-specific epitopes of islet-specific glucose-6-phosphatase-catalytic subunit-related protein: a novel beta cell autoantigen in type 1 diabetes. J. Immunol. 174, 5306-5315 (2005).
29 Kamanaka, M. et al. Expression of interleukin-10 in intestinal lymphocytes detected by an interleukin-10 reporter knockin tiger mouse. Immunity 25, 941-952 (2006).
30 Yoshizaki, A. et al. Regulatory B cells control T-cell autoimmunity through IL-21-dependent cognate interactions. Nature 491, 264-268 (2012).
31 Onoda, T. et al. Human CD4+central and effector memory T cells produce IL-21: effect on cytokine-driven proliferation of CD4+ T cell subsets. Int. Immunol. 19, 1191-1199 (2007).
32 Pot, C. et al. Cutting edge: IL-27 induces the transcription factor c-Maf, cytokine IL-21, and the costimulatory receptor ICOS that coordinately act together to promote differentiation of IL-10-producing T_{R}1 cells. J. Immunol. 183, 797-801 (2009).
33 Spensieri, F. et al. Human circulating influenza-CD4+ICOS1+IL-21+ T cells expand after vaccination, exert helper function, and predict antibody responses. Proc. Natl. Acad. Sci. U.S.A. 110, 14330-14335 (2013).
34 Hale, J. S. et al. Distinct memory CD4+ T cells with commitment to T follicular helper- and T helper 1-cell lineages are generated after acute viral infection. Immunity 38, 805-817 (2013).
35 Sato, K. et al. Marked induction of c-Maf protein during Th17 cell differentiation and its implication in memory Th cell development. J. Biol. Chem. 286, 14963-14971 (2011).
36 Saraiva, M. et al. Interleukin-10 production by Th1 cells requires interleukin-12-induced STAT4 transcription factor and ERK MAP kinase activation by high antigen dose. Immunity 31, 209-219 (2009).
37 Verdaguer, J. et al. Spontaneous autoimmune diabetes in monoclonal T cell nonobese diabetic mice. J. Exp. Med. 186, 1663-1676 (1997).
38 Wang, J. et al. In situ recognition of autoantigen as an essential gatekeeper in autoimmune CD8+ T cell inflammation. Proc. Natl. Acad. Sci. U.S.A. 107, 9317-9322 (2010).
39 Amrani, A. et al. Progression of autoimmune diabetes driven by avidity maturation of a T-cell population. Nature 406, 739-742 (2000).
40 Stratmann, T. et al. The I-Ag7 MHC class II molecule linked to murine diabetes is a promiscuous peptide binder. J. Immunol. 165, 3214-3225 (2000).
41 Yang, J. et al. Islet-specific glucose-6-phosphatase catalytic subunit-related protein-reactive CD4⁺ T cells in human subjects. J. Immunol. 176, 2781-2789 (2006).
42 Holst, J. et al. Generation of T-cell receptor retrogenic mice. Nat. Protoc. 1, 406-417 (2006).
43 Yoshida, K. et al. Evidence for shared recognition of a peptide ligand by a diverse panel of non-obese diabetic mice-derived, islet-specific, diabetogenic T cell clones. Int. Immunol. 14, 1439-1447 (2002).
44 Reijonen, H. et al. Detection of GAD65-specific T-cells by major histocompatibility complex class II tetramers in type 1 diabetic patients and at-risk subjects. Diabetes 51, 1375-1382 (2002).
45 Yang, J. et al. CD4+ T cells from type 1 diabetic and healthy subjects exhibit different thresholds of activation to a naturally processed proinsulin epitope. J. Autoimmun. 31, 30-41 (2008).
46 Moore, A., Grimm, J., Han, B. & Santamaria, P. Tracking the recruitment of diabetogenic CD8+ T cells to the pancreas in real time. Diabetes 53, 1459-1466 (2004).
47 Giuliani, F. et al. Additive effect of the combination of glatiramer acetate and minocycline in a model of MS. J. Neuroimmunol. 158, 213-221 (2005).
48 Leavenworth, J. W., Tang, X., Kim, H. J., Wang, X. & Cantor, H. Amelioration of arthritis through mobilization of peptide-specific CD8+regulatory T cells. J. Clin. Invest. 123, 1382-1389 (2013).

What is claimed is:

1. A complex comprising a nanoparticle core coupled to a plurality of disease-relevant antigen-MHC (pMHC) complexes,
wherein the nanoparticle core has a diameter selected from the group of: from about 1 nm to about 100 nm; from about 1 nm to about 50 nm; from about 1 nm to about 25 nm; from about 5 nm to about 100 nm; from about 5 nm to about 50 nm; or from about 5 nm to about 25 nm;
wherein the pMHC density per nanoparticle is from about 0.025 pMHC/100 nm² to about 100 pMHC/100 nm² of the surface area of the nanoparticle, and
optionally wherein the nanoparticle core further comprises an outer layer on the nanoparticle core.

2. The complex of claim 1, further comprising a plurality of co-stimulatory molecules and/or a plurality of cytokines coupled to the nanoparticle core and wherein the valency of the co-stimulatory molecules is from about 1 to about 6000, and/or the valency of the cytokines is from about 1 to about 6000, each per nanoparticle core.

3. The complex of claim 1 or 2, wherein the plurality of pMHC complexes are the same or different from each other; and/or the plurality of co-stimulatory molecules are the same or different from each other; and/or the plurality of cytokines are the same or different from each other.

4. The complex of any of claims 1 to 3, wherein the MHC protein of the pMHC complexes are one or more of the group of classical MHC class I protein, non-classical MHC class I protein, classical MHC class II protein, non-classical MHC class II protein, MHC dimers (Fc fusions), MHC tetramers, or a polymeric form of a MHC protein, that may be the same or different from each other on the nanoparticle core, wherein the MHC protein optionally comprises a knob-in-hole based MHC-alpha-Fc/MHC-beta-Fc heterodimer or multimer.

5. The complex of any of claims 1 to 4, wherein the antigens of the plurality of pMHCs are the same or different from each other.

6. The complex of any of claims 1 to 5, wherein the disease-relevant antigen of the pMHC complexes is selected from an autoimmune disease-relevant antigen, an inflammation-relevant antigen, an allergic disease-relevant antigen, a cancer-relevant antigen, or a tumor-relevant antigen.

7. The complex of claim 6, wherein the immune inflammation-relevant antigen is one or more of the group: an asthma-relevant antigen, a diabetes-relevant antigen, a pre-diabetes relevant antigen, a multiple sclerosis-relevant antigen, an allergic asthma-relevant antigen, a primary biliary cirrhosis-relevant antigen, a cirrhosis-relevant antigen, a Neuromyelitis optica spectrum disorder (Devic's disease, NMO)-relevant antigen, an autoimmune encephalitis-relevant antigen, an antigen relevant to autoantibody-mediated neurological syndromes, a Stiff Man syndrome-relevant antigen, a paraneoplastic disease-relevant antigen, antigens relevant to other diseases of the central and peripheral nervous systems, a Pemphigus vulgaris-relevant antigen, inflammatory bowel disease (IBD)-relevant antigen, Crohn's disease-relevant antigen, Ulcerative Colitis-relevant antigen, an arthritis-relevant antigen, a Rheumatoid Arthritis-relevant antigen, a systemic lupus erythematosus (SLE)-relevant antigen, a Celiac Disease relevant antigen, a psoriasis-relevant antigen, an Alopecia Areata-relevant antigen, an Acquired Thrombocytopenic Purpura-relevant antigen, an autoimmune cardiomyopathy-relevant antigen, an idiopathic dilated cardiomyopathy (IDCM)-relevant antigen, a Myasthyenia Gravis-relevant antigen, an Uveitis-relevant antigen, an Ankylosing Spondylitis-relevant antigen, a Grave's Disease-relevant antigen, a Hashimoto's thyroiditis-relevant antigen, an Immune Mediated Myopathies-relevant antigen, an anti-phospholipid syndrome (ANCA+)-relevant antigen, an atherosclerosis-relevant antigen, a scleroderma-relevant antigen, an autoimmune hepatitis-relevant antigen, a dermatomyositis-relevant antigen, a chronic obstructive pulmonary disease-relevant antigen, a spinal cord injury-relevant antigen, a traumatic injury-relevant antigen, a tobacco-induced lung destruction-relevant antigen, a Chronic Obstructive Pulmonary Disease (COPD)-relevant antigen, a lung emphysema-relevant antigen, a sclerosing cholangitis-relevant antigen, a peripheral neuropathy-relevant antigen, a narcolepsy-relevant antigen, a Goodpasture Syndrome-relevant antigen, a Kawasaki's Disease-relevant antigen, an autoimmune uveitis-relevant antigen, a colitis-relevant antigen, an emphysema-relevant antigen, a pemphigus-relevant antigen, a pemphigus folliaceus-relevant antigen, an arthritis-relevant antigen, a Sjogren's Syndrome-relevant antigen, an ANCA-associated vasculitis-relevant antigen, a primary sclerosing cholangitis-relevant antigen, an adipose tissue inflammation/diabetes type II-relevant antigen, or an obesity associated adipose tissue inflammation/insulin resistance-relevant antigen.

8. The complex of any of claims 1-7, wherein the nanoparticle core comprises a core selected from the group of: a solid core, a metal core, a dendrimer core, a polymeric micelle nanoparticle core, a nanorod, a fullerene, a nanoshell, a coreshell, a protein-based nanostructure or a lipid-based nanostructure, optionally a non-liposomal core.

9. The complex of any of claims 1-8, wherein the nanoparticle core is non-liposomal.

10. The complex of any of claims 1-9, wherein the pMHC complex is coupled to the nanoparticle core by one or more of covalently, non-covalently, or cross-linked and optionally coupled through a linker to the nanoparticle core or the outer layer.

11. The complex of claim 10, wherein the linker is less than 5 kD in size, that is optionally polyethylene glycol.

12. The complex of claim 10 or 11, wherein the linkers are the same or different from each other on the nanoparticle core.

13. The complex of any of claims 1-12, wherein the outer layer comprises polyethylene glycol.

14. The complex of any of claims 1-13, wherein the nanoparticle core and/or the outer layer on the nanoparticle core is bioabsorbable and/or biodegradable.

15. The complex of any of claims 1-14, wherein the valency of the pMHC complexes per nanoparticle core is from about 10:1 to about 6000:1.

16. The complex of any of claims 1-15, wherein the density of the pMHC complexes per nanoparticle comprises from about 0.4 pMHC/100 nm² of surface area of the nanoparticle to about 50 pMHC/100 nm² of surface area of the nanoparticle.

17. The complex of any of claims 1-16, wherein the MHC molecule of the pMHC complex comprises any loci of HLA DR, HLA DQ, HLA DP, HLA-A, HLA-B, HLA-C, HLA-E, HLA-F, HLA-G, CD1d, or a fragment or a biological equivalent of each thereof.

18. The complex of any of claims 1-17, wherein the valency of the pMHC complexes per nanoparticle core is from about 10:1 to about 100:1.

19. The complex of any of claims 1-18, wherein the pMHC density is from about 0.4 pMHC/100 nm² to about 6 pMHC/100 nm².

20. The complex of any of claims 1-19, wherein the disease-relevant antigen is:
a) a diabetes-relevant antigen and is derived from an antigen selected from one or more of the group: preproinsulin (PPI), islet-specific glucose-6-phosphatase (IGRP), glutamate decarboxylase (GAD), islet cell autoantigen-2 (ICA2), insulin, proinsulin, or a fragment or an equivalent of each thereof;
b) a multiple sclerosis-relevant antigen and is derived from an antigen selected from one or more of the group: myelin basic protein, myelin associated glycoprotein, myelin oligodendrocyte protein, proteolipid protein, oligodendrocyte myelin oligoprotein, myelin associated oligodendrocyte basic protein, oligodendrocyte specific protein, heat shock proteins, oligodendrocyte specific proteins, NOGO A, glycoprotein Po, peripheral myelin protein 22, 2′3′-cyclic nucleotide 3′-phosphodiesterase, or a fragment or an equivalent of each thereof;
c) a Celiac Disease-relevant antigen and is derived from gliadin or a fragment or an equivalent thereof;
d) a primary biliary cirrhosis-relevant antigen and is derived from PDC-E2 or a fragment or an equivalent thereof;
e) a pemphigus folliaceus-relevant antigen and/or pemphigus vulgaris-relevant antigen and is derived from an antigen selected from one or more of the group: DG1, DG3, or a fragment or an equivalent of each thereof;
f) a neuromyelitis optica spectrum disorder-relevant antigen and is derived from AQP4 or a fragment or an equivalent thereof;
g) an arthritis-relevant antigen and is derived from an antigen selected from one or more of the group: heat shock proteins, immunoglobulin binding protein, heterogeneous nuclear RNPs, annexin V, calpastatin, type II collagen, glucose-6-phosphate isomerase, elongation factor human cartilage gp39, mannose binding lectin, citrullinated vimentin, type II collagen, fibrinogen, alpha enolase, anti-carbamylated protein (anti-CarP), peptidyl arginine deiminase type 4 (PAD4), BRAF, fibrinogen gamma chain, inter-alpha-trypsin inhibitor heavy chain H1, alpha-1-antitrypsin, plasma protease C1 inhibitor, gelsolin, alpha 1-B glycoprotein, ceruloplasmin, inter-alpha-trypsin inhibitor heavy chain H4, complement factor H, alpha 2 macroglobulin, serum amyloid, C-reactive protein, serum albumin, fibrogen beta chain, serotransferin, alpha 2 HS glycoprotein, vimentin, Complement C3, or a fragment or an equivalent of each thereof;
h) an allergic asthma-relevant antigen and is derived from an antigen selected from one or more of the group: DERP1, DERP2, or a fragment or an equivalent of each thereof;
i) an inflammatory bowel disease-relevant antigen and is derived from an antigen selected from one or more of the group: Flagelin, Fla-2, Fla-X, YIDX, bacteroides integrase, or a fragment or an equivalent of each thereof;
j) a systemic lupus erythematosus-relevant antigen and is derived from an antigen selected from one or more of the group: double-stranded (ds)DNA, ribonucleoprotein (RNP), Smith (Sm), Sjögren's-syndrome-related antigen A (SS-A)/Ro, Sjögren's-syndrome-related antigen B (SS-B)/La, RO60, RO52, histones, or a fragment or an equivalent of each thereof;
k) an atherosclerosis-relevant antigen and is derived from an antigen selected from one or more of the group: ApoB, ApoE or a fragment or an equivalent of each thereof;
l) a COPD-relvant antigen and/or emphysema-relevant antigen and is derived from elastin or a fragment or an equivalent thereof;
m) a psoriasis-relevant antigen and is derived from an antigen selected from one or more of the group: Cap18, ADMTSL5, ATL5, or a fragment or an equivalent of each thereof;
n) an autoimmune hepatitis-relevant antigen and is derived from an antigen selected from one or more of the group: CYP2D6, SLA, or a fragment or an equivalent of each thereof;
o) an uveitis-relevant antigen and is derived from arrestin or a fragment or an equivalent thereof;
p) a Sjogren's Syndrome-relevant antigen and is derived from an antigen selected from one or more of the group: (SS-A)/Ro, (SS-B)/La, MR3, RO60, RO52, or a fragment or an equivalent of each thereof;
q) a scleroderma-relevant antigen and is derived from an antigen selected from one or more of the group: CENP-C, TOP 1, RNA polymerase III, or a fragment or an equivalent of each thereof;
r) an anti-phospholipid syndrome-relevant antigen and is derived from APOH or a fragment or an equivalent thereof;
s) an ANCA-associated vasculitis-relevant antigen and is derived from an antigen selected from one or more of the group: MPO, PRTN3, or a fragment or an equivalent of each thereof; or
t) a Stiff Man Syndrome-relevant antigen and is derived from GAD or a fragment or an equivalent thereof.

21. The complex of any of claims 5-20, wherein the MHC protein of the pMHC complex comprises all or part of a classical MHC class I protein, non-classical MHC class I protein, classical MHC class II protein, non-classical MHC class II protein, MHC dimers (Fc fusions), MHC tetramers, or a polymeric form of a MHC protein, wherein the MHC protein optionally comprises a knob-in-hole based MHC-alpha-Fc/MHC-beta-Fc heterodimer or multimer.

22. The complex of any of claims 1-17 and 19-21, wherein the MHC protein of the pMHC complex comprises all or part of a polypeptide of the group: HLA DR, HLA DQ, HLA DP, HLA-A, HLA-B, HLA-C, HLA-E, HLA-F, HLA-G, CD1d, or a fragment or an equivalent of each thereof.

23. The complex of any of claims 1-22, wherein the MHC protein of the pMHC complex comprises all or part of a polypeptide of the group: HLA-DR, HLA-DQ, HLA-DP, or a fragment or an equivalent of each thereof.

24. The complex of any of claims 1-23, wherein the MHC protein of the pMHC complex comprises all or part of a polypeptide of the group: HLA-DRB1/DRA, HLA-DRB3/DRA, HLA-DRB4/DRA, HLA-DRB5/DRA, HLA-DQA1/HLA-DQB1, HLA-DPB1/HLA-DPA1, or a fragment or an equivalent of each thereof.

25. The complex of any of claims 1-24, wherein the pMHC complex comprises:
a) a diabetes-relevant antigen derived from an antigen selected from one or more of the group: hInsB₁₀₋₁₈, hIGRP₂₂₈₋₂₃₆, hIGRP₂₆₅₋₂₇₃, IGRP₂₀₆₋₂₁₄, hIGRP₂₀₆₋₂₁₄, NRP-A7, NRP-I4, NRP-V7, YAI/D^{b}, INS B₁₅₋₂₃, PPI_{76-90 (K88S)}, IGRP₁₃₋₂₅, GAD₅₅₅₋₅₆₇, GAD_{555-567(557I)}, IGRP₂₃₋₃₅, B₂₄-C₃₆, PPI₇₆₋₉₀, INS-I9, TUM, G6Pase, Pro-insulin_{L2-10}, Pro-insulin_{L3-11}, Pro-insulin_{L6-14}, Pro-insulin_{B5-14}, Pro-insulin_{B10-18}, Pro-insulin_{B14-22}, Pro-insulin_{B15-24}, Pro-insulin_{B17-25}, Pro-insulin_{B18-27}, Pro-insulin_{B20-27}, Pro-insulin_{B21-29}, Pro-insulin_{B25-C1}, Pro-insulin_{B27-C5}, Pro-insulin_{C20-28}, Pro-insulin_{C25-33}, Pro-insulin_{C29-A5}, Pro-insulin_{A1-10}, Pro-insulin_{A2-10}, Pro-insulin_{A12-20}, or a fragment or an equivalent of each thereof;
b) a multiple sclerosis-relevant antigen derived from an antigen selected from one or more of the group: MOG₃₅₋₅₅, MOG₃₆₋₅₅, MAG₂₈₇₋₂₉₅, MAG₅₀₉₋₅₁₇, MAG₅₅₆₋₅₆₄, MBP₁₁₀₋₁₁₈, MOG₁₁₄₋₁₂₂, MOG₁₆₆₋₁₇₅, MOG₁₇₂₋₁₈₀, MOG₁₇₉₋₁₈₈, MOG₁₈₈₋₁₉₆, MOG₁₈₁₋₁₈₉, MOG₂₀₅₋₂₁₄, PLP₈₀₋₈₈, MAG₂₈₇₋₂₉₅, MAG₅₀₉₋₅₁₇, MAG₅₅₆₋₅₆₄, MOG₉₇₋₁₀₉ MOG_{97-109(E107S)}, MBP₉₋₁₀₁, PLP₁₇₅₋₁₉₂, PLP₉₄₋₁₀₈, MBP₈₆₋₉₈, PLP₅₄₋₆₈, PLP₂₄₉₋₂₆₃, MOG₁₅₆₋₁₇₀, MOG₂₀₁₋₂₁₅, MOG₃₈₋₅₂, MOG₂₀₃₋₂₁₇, PLP₂₅₀₋₂₆₄, MPB₁₃₋₃₂, MPB₈₃₋₉₉, MPB₁₁₁₋₁₂₉, MPB₁₄₆₋₁₇₀, MOG₂₂₃₋₂₃₇, MOG₆₋₂₀, PLP₈₈₋₁₀₂, PLP₁₃₉₋₁₅₄, or a fragment or an equivalent of each thereof;
c) a Celiac Disease-relevant antigen derived from an antigen selected from one or more of the group: aGlia₅₇₋₅₅, aGlia₆₂₋₇₂, aGlia₂₁₇₋₂₂₉, or a fragment or an equivalent of each thereof;
d) a primary biliary cirrhosis-relevant antigen derived from an antigen selected from one or more of the group: PDC-E2₁₂₂₋₁₃₅, PDC-E2₂₄₉₋₂₆₂, PDC-E2₂₄₉₋₂₆₃, PDC-E2₆₂₉₋₆₄₃, PDC-E₂₇₂₋₈₆, PDC-E2₃₅₃₋₃₆₇, PDC-E2₄₂₂₋₄₃₆, PDC-E2₆₂₉₋₆₄₃, PDC-E2₈₀₋₉₄, PDC-E2₃₅₃₋₃₆₇, PDC-E₂₅₃₅₋₅₄₉, or a fragment or an equivalent of each thereof;
e) a pemphigus folliaceus-relevant antigen and/or pemphigus vulgaris-relevant antigen, each of which is derived from an antigen selected from one or more of the group: DG1₂₁₆₋₂₂₉, DG3₉₇₋₁₁₁, DG3₂₅₁₋₂₆₅, DG3₄₄₁₋₄₅₅, DG3₃₅₁₋₃₆₅, DG3₄₅₃₋₄₆₇, DG3₅₄₀₋₅₅₄, DG3₂₈₀₋₂₉₄, DG3₃₂₆₋₃₄₀, DG3₃₆₇₋₃₈₁, DG3₁₃₋₂₇, DG3₃₂₃₋₃₃₇, DG3₄₃₈₋₄₅₂, DG1₄₈₋₆₂, DG1₂₀₆₋₂₂₂, DG1₃₆₃₋₃₇₇, DG1₃₋₁₇, DG1₁₉₂₋₂₀₆, DG1₃₂₆₋₃₄₀, DG1₁₋₁₅, DG1₃₅₋₄₉, DG1₃₂₅₋₃₃₉, or a fragment or an equivalent of each thereof;
f) a neuromyelitis optica spectrum disorder-relevant antigen derived from an antigen selected from one or more of the group: AQP4₁₂₉₋₁₄₃, AQP4₂₈₄₋₂₉₈, AQP4₆₃₋₇₆, AQP4₁₂₉-143, AQP4₃₉₋₅₃, or a fragment or an equivalent of each thereof;
g) an allergic asthma-relevant antigen derived from an antigen selected from one or more of the group: DERP1₁₆₋₃₀, DERP1₁₇₁₋₈₅, DERP1₁₁₀₋₁₂₄, DERP-2₂₆₋₄₀, DERP-2₁₀₇₋₁₂₁, or a fragment or an equivalent of each thereof;
h) an inflammatory bowel disease-relevant antigen derived from an antigen selected from one or more of the group: bacteroides integrase antigen₁₈₃₋₁₉₇, bacteroides integrase antigen₁₄₆₋₁₆₀, bacteroides integrase antigen₁₇₅₋₆₅₉, bacteroides integrase antigen₁₋₁₅, bacteroides integrase antigen₁₈₃₋₁₉₇, bacteroides integrase antigen₃₀₋₄₄, bacteroides integrase antigen₇₇₋₈₄, bacteroides integrase antigen₃₃₇₋₃₅₁, bacteroides integrase antigen₁₇₁₋₁₈₅, bacteroides integrase antigen₄₋₁₈, bacteroides integrase antigen₂₆₋₂₇₀, Fla-2/Fla-X₃₆₆₋₃₈₀, Fla-2/Fla-X₁₆₄₋₁₇₈, Fla-2/Fla-X₂₆₁₋₂₇₅, Fla-2/Fla-X₁₋₁₅, Fla-2/Fla-X₅₁₋₆s, Fla-2/Fla-X₂₆₉₋₂₈₃, Fla-2/Fla-X₄₋₁₈, Fla-2/Fla-X₂₇₁₋₂₈₅, YIDX₇₈₋₉₂, YIDX₉₃₋₁₀₇, YIDX₉₈₋₁₁₂, YIDX₂₃₋₃₇, YIDX₇₈₋₉₂, YIDX₁₉₅₋₂₀₉, YIDX₂₂₋₃₆, YIDX₈₀₋₉₄, YIDX₁₀₁₋₁₁₅, or a fragment or an equivalent of each thereof;
i) a systemic lupus erythematosus-relevant antigen derived from an antigen selected from one or more of the group: H4₇₁₋₉₄, H4₇₄₋₈₈, H4₇₆₋₉₀, H4₇₅₋₈₉, H4₇₈₋₉₂, H4₈₀₋₉₄, H2B₁₀₋₂₄, H2B₁₆₋₃₀, H1′₂₂₋₄₂, H1′₂₇₋₄₁, or a fragment or an equivalent of each thereof;
j) an atherosclerosis-relevant antigen derived from an antigen selected from one or more of the group: ApoB₃₅₀₁₋₃₅₁₆, ApoB₁₉₅₂₋₁₉₆₆, ApoB₉₇₈₋₉₉₃, ApoB₃₄₉₈₋₃₅₁₃, ApoB_{210A}, ApoB_{210B}, ApoB_{210C}, or a fragment or an equivalent of each thereof;
k) a COPD-relvant antigen and/or emphysema-relevant antigen, each of which is derived from an antigen selected from one or more of the group: elastin₈₉₋₁₀₃, elastin₆₉₈₋₇₁₂, elastin₈₋₂₂, elastin₉₄₋₁₀₈, elastin₁₃₋₂₇, elastin₆₉₅₋₇₀₉, elastin₅₆₃₋₅₇₇, elastin₅₅₈₋₅₇₂, elastin₆₉₈₋₇₁₂, elastin₅₆₆₋₅₈₀, elastin₅₄₅₋₆₅₉, or a fragment or an equivalent of each thereof;
l) a psoriasis-relevant antigen derived from an antigen selected from one or more of the group: Cap18₆₄₋₇₈, Cap18₃₄₋₄₈, Cap18₄₇₋₆₁, Cap18₁₅₁₋₁₆₅, Cap18₁₄₉₋₁₆₃, Cap18₁₅₂₋₁₆₆, Cap18₁₃₁₋₁₄₅, Cap18₂₄₋₃₈, ADMTSL5245₋₂₅₉, ADMTSL5₂₆₇₋₂₈₁, ADMTSL5₃₇₂₋₃₈₆, ADMTSL5₂₈₉₋₃₀₃, ADMTSL5₃₉₆₋₄₁₀, ADMTSL5₄₃₃₋₄₄₇, ADMTSL5₁₄₂₋₁₅₆, ADMTSL5₂₃₆₋₂₅₀, ADMTSL5₃₀₁₋₃₁₅, ADMTSL5₂₀₃₋₂₁₇, ADMTSL5₄₀₄₋₄₁₈, or a fragment or an equivalent of each thereof;
m) an autoimmune hepatitis-relevant antigen derived from an antigen selected from one or more of the group: (CYP2D6)₁₉₃₋₂₀₇, CYP2D6₇₆₋₉₀, CYP2D6₂₉₃₋₃₀₇, CYP2D6₃₁₃₋₃₃₂, CYP2D6₃₉₃₋₄₁₂, CYP2D6₁₉₉₋₂₁₃, CYP2D6₄₅₀₋₄₆₄, CYP2D6₃₀₁₋₃₁₅, CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₃₁₋₁₄₅, CYP2D6₂₁₆₋₂₃₀, CYP2D6₂₃₈₋₂₅₂, CYP2D6₁₉₉₋₂₁₃, CYP2D6₂₃₅₋₂₅₂, CYP2D6₂₉₃₋₃₀₇, CYP2D6₃₈₁₋₃₉₅, CYP2D6₄₂₉₋₄₄₃, SLA₃₃₄₋₃₄₈, SLA₁₉₆₋₂₁₀, SLA₁₁₅₋₁₂₉, SLA₃₇₃₋₃₈₆, SLA₁₈₆₋₁₉₇, SLA₃₁₇₋₃₃₁, SLA₁₇₁₋₁₈₅, SLA₄₁₇₋₄₃₁, SLA₃₅₉₋₃₇₃, SLA₂₁₅₋₂₂₉, SLA₁₁₁₋₁₂₅, SLA₁₁₀₋₁₂₄, SLA₂₉₉₋₃₁₃, SLA₃₄₂₋₃₅₆, SLA₄₉₋₆₃, SLA₁₁₉₋₁₃₃, SLA₂₆₀₋₂₇₄, SLA₂₆₋₄₀, SLA₈₆₋₁₀₀, SLA₃₃₁₋₃₄₅, or a fragment or an equivalent of each thereof;
n) an uveitis-relevant antigen derived from an antigen selected from one or more of the group: arrestin₁₉₉₋₂₁₃, arrestin₇₇₋₉₁, arrestin₂₅₀₋₂₆₄, arrestin₁₇₂₋₁₈₆, arrestin₃₅₄₋₃₆₈, arrestin₂₃₉₋₂₅₃, arrestin₁₀₂₋₁₁₆, arrestin₅₉₋₇₃, arrestin₂₈₀₋₂₉₄, arrestin₂₉₁₋₃₀₆, arrestin₁₉₅₋₂₀₉, arrestin₂₀₀₋₂₁₄, or a fragment or an equivalent of each thereof;
o) a Sjogren's Syndrome-relevant antigen derived from an antigen selected from one or more of the group: RO60₁₂₇₋₁₄₁, RO60₅₂₃₋₅₃₇, RO60₂₄₃₋₂₅₇, RO60₄₈₄₋₄₉₈, RO60₃₄₇₋₃₆₁, RO60₃₆₉₋₃₈₃, RO60₄₂₆₋₄₄₀, RO60₂₆₇₋₂₈₁, RO60₁₇₈₋₁₉₂, RO60₃₅₈₋₃₇₂, RO60₂₂₁₋₂₃₅, RO60₃₁₈₋₃₃₂, RO60₄₀₇₋₄₂₁, RO60₄₅₉₋₄₇₃, RO60₅₁₋₆₅, RO60₃₁₂₋₃₂₆, LA₂₄₁₋₂₅₅, LA₁₀₁₋₁₁₅, LA₁₅₃₋₁₆₇, LA₁₇₈₋₁₉₂, LA₁₉₋₃₃, LA₃₇₋₅₁, LA₁₃₃₋₁₄₇, LA₅₀₋₆₄, LA₃₂₋₄₆, LA₁₅₃₋₁₆₇, LA₈₃₋₉₇, LA₁₃₆₋₁₅₀, LA₂₉₇₋₃₁₁, LA₅₉₋₇₃, LA₁₅₁₋₁₆₅, LA₈₆₋₁₀₀, LA₁₅₄₋₁₆₈, or a fragment or an equivalent of each thereof;
p) a scleroderma-relevant antigen derived from an antigen selected from one or more of the group: TOP1₃₄₆₋₃₆₀, TOP1₄₂₀₋₄₃₄, TOP1₇₅₀₋₇₆₄, TOP1₄₁₉₋₄₃₃, TOP1₅₉₁₋₆₀₅, TOP1₆₉₅₋₇₀₉, TOP1₃₀₅₋₃₁₉, TOP1₃₄₆₋₃₆₀, TOP1₄₁₉₋₄₃₃, TOP1₄₂₅₋₄₃₉, TOP1₆₁₄₋₆₂₈, CENP-C₂₉₇₋₃₁₁, CENP-C₈₅₇₋₈₇₁, CENP-C₈₈₇₋₉₀₁, CENP-C₂₁₂₋₂₂₆, CENP-C₆₄₃₋₆₅₇, CENP-C₈₃₂₋₈₄₆, CENP-C₁₆₇₋₁₈₁, CENP-C₂₄₆₋₂₆₀, CENP-C₈₄₆₋₈₆₀, CENP-C₁₄₉₋₁₆₃, CENP-C₈₃₃₋₈₄₇, CENP-C₈₄₇₋₈₆₁, or a fragment or an equivalent of each thereof;
q) an anti-phospholipid syndrome-relevant antigen derived from an antigen selected from one or more of the group: APOH₂₃₅₋₂₄₉, APOH₃₀₆₋₃₂₀, APOH₂₃₇₋₂₅₁, APOH₂₉₅₋₃₀₉, APOH₂₈₄₂, APOH₁₇₃₋₁₈₇, APOH₂₆₄₋₂₇₈, APOH₂₉₅₋₃₀₉, APOH₄₉₋₆₃, APOH₂₆₉₋₂₈₃, APOH₂₉₅₋₃₀₉, APOH₃₂₁₋₃₅₅, APOH₃₂₂₋₃₃₆, APOH₃₂₄₋₃₃₈, or a fragment or an equivalent of each thereof;
r) an ANCA-associated vasculitis-relevant antigen derived from an antigen selected from one or more of the group: MPO₅₀₆₋₅₂₀, MPO₃₀₂₋₃₁₆, MPO₇₋₂₁, MPO₆₈₉₋₇₀₃, MPO₂₄₈₋₂₆₂, MPO₄₄₄₋₄₅₈, MPO₅₁₃₋₅₂₇, MPO₉₇₋₁₁₁, MPO₆₁₆₋₆₃₀, MPO₄₆₂₋₄₇₆, MPO₆₁₇₋₆₃₁, MPO₇₁₄₋₇₂₈, PRTN3₄₄₋₅₈, PRTN3₂₃₄₋₂₄₈, PRTN3₅₉₋₇₃, PRTN3₁₁₇₋₁₃₁, PRTN3₁₆₄₋₁₇₈, PRTN3₇₁₋₈₅, PRTN3₂₄₁₋₂₅₅, PRTN3₅₉₋₇₃, PRTN3₁₈₃₋₁₉₇, PRTN3₆₂₋₇₆, PRTN3₁₁₈₋₁₃₂, PRTN3₂₃₉₋₂₅₃, or a fragment or an equivalent of each thereof; or
s) a Stiff Man Syndrome-relevant antigen derived from an antigen selected from one or more of the group: GAD₂₁₂₋₂₂₆, GAD₅₅₅₋₅₆₉, GAD₂₉₇₋₃₁₁, or a fragment or an equivalent of each thereof.

26. The complex of any of claims 1-25, wherein the pMHC complex comprises:
a) a diabetes-relevant antigen derived from an antigen selected from one or more of the group: hInsB₁₀₋₁₈, hIGRP₂₂₈₋₂₃₆, hIGRP₂₆₅₋₂₇₃, IGRP₂₀₆₋₂₁₄, hIGRP₂₀₆₋₂₁₄, NRP-A7, NRP-14, NRP-V7, YAI/D^{b}, INS B₁₅₋₂₃, PPI_{76-90 (K88S)}, IGRP₁₃₋₂₅, GAD₅₅₅₋₅₆₇, GAD_{555-567(557I)}, IGRP₂₃₋₃₅, B₂₄-C₃₆, PPI₇₆₋₉₀, INS-I9, TUM, G6Pase, Pro-insulin_{L2-10}, Pro-insulin_{L3-11}, Pro-insulin_{L6-14}, Pro-insulin_{B5-14}, Pro-insulin_{B10-18}, Pro-insulin_{B14-22}, Pro-insulin_{B15-24}, Pro-insulin_{B17-25}, Pro-insulin_{B18-27}, Pro-insulin_{B20-27}, Pro-insulin_{B21-29}, Pro-insulin_{B25-C1}, Pro-insulin_{B27-C5}, Pro-insulin_{C20-28}, Pro-insulin_{C25}-33, Pro-insulin_{C29-A5}, Pro-insulin_{A1-10}, Pro-insulin_{A2-10}, Pro-insulin_{A12-20}, or a fragment or an equivalent of each thereof, and the MHC protein of the pMHC complex comprises all or part of HLA-DR or a fragment or an equivalent thereof;
b) a multiple sclerosis-relevant antigen derived from an antigen selected from one or more of the group: MOG₃₅₋₅₅, MOG₃₆₋₅₅, MAG₂₈₇₋₂₉₅, MAG₅₀₉₋₅₁₇, MAG₅₅₆₋₅₆₄, MBP₁₁₀₋₁₁₈, MOG₁₁₄₋₁₂₂, MOG₁₆₆₋₁₇₅, MOG₁₇₂₋₁₈₀, MOG₁₇₉₋₁₈₈, MOG₁₈₈₋₁₉₆, MOG₁₈₁₋₁₈₉, MOG₂₀₅₋₂₁₄, PLP₈₀₋₈₈, MAG₂₈₇₋₂₉₅, MAG₅₀₉₋₅₁₇, MAG₅₅₆₋₅₆₄, MOG₉₇₋₁₀₉ MOG_{97-109(E107S)}, MBP₈₉₋₁₀₁, PLP₁₇₅₋₁₉₂, PLP₉₄₋₁₀₈, MBP₈₆₋₉₈, PLP₅₄₋₆₈, PLP₂₄₉₋₂₆₃, MOG₁₅₆₋₁₇₀, MOG₂₀₁₋₂₁₅, MOG₃₈₋₅₂, MOG₂₀₃₋₂₁₇, PLP₂₅₀₋₂₆₄, MPB₁₃₋₃₂, MPB₈₃₋₉₉, MPB₁₁₁₋₁₂₉, MPB₁₄₆₋₁₇₀, MOG₂₂₃₋₂₃₇, MOG₆₋₂₀, PLP₈₈₋₁₀₂, PLP₁₃₉₋₁₅₄, or a fragment or an equivalent of each thereof, and the MHC protein of the pMHC complex comprises all or part of HLA-DR or a fragment or an equivalent thereof;
c) a Celiac Disease-relevant antigen derived from an antigen selected from one or more of the group: aGlia₅₇₋₆₈, aGlia₆₂₋₇₂, aGlia₂₁₇₋₂₂₉, or a fragment or an equivalent of each thereof, and the MHC protein of the pMHC complex comprises all or part of HLA-DQ or a fragment or an equivalent thereof;
d) a primary biliary cirrhosis-relevant antigen derived from an antigen selected from one or more of the group: PDC-E2₁₂₂₋₁₃₅, PDC-E2₂₄₉₋₂₆₂, PDC-E2₂₄₉₋₂₆₃, PDC-E2₆₂₉₋₆₄₃, PDC-E₂₇₂₋₈₆, PDC-E2₃₅₃₋₃₆₇, PDC-E2₄₂₂₋₄₃₆, PDC-E2₆₂₉₋₆₄₃, PDC-E2₈₀₋₉₄, PDC-E2₃₅₃₋₃₆₇, PDC-E₂₅₃₅₋₅₄₉, or a fragment or an equivalent of each thereof, and the MHC protein of the pMHC complex comprises all or part of HLA-DR or a fragment of an equivalent thereof;
e) a pemphigus folliaceus-relevant antigen and/or pemphigus vulgaris-relevant antigen, each of which is derived from an antigen selected from one or more of the group: DG1₂₁₆₋₂₂₉, DG3₉₇₋₁₁₁, DG3₂₅₁₋₂₆₅, DG3₄₄₁₋₄₅₅, DG3₃₅₁₋₃₆₅, DG3₄₅₃₋₄₆₇, DG3₅₄₀₋₅₅₄, DG3₂₈₀₋₂₉₄, DG3₃₂₆₋₃₄₀, DG3₃₆₇₋₃₈₁, DG3₁₃₋₂₇, DG3₃₂₃₋₃₃₇, DG3₄₃₈₋₄₅₂, DG1₄₈₋₆₂, DG1₂₀₆₋₂₂₂, DG1₃₆₃₋₃₇₇, DG1₃₋₁₇, DG1₁₉₂₋₂₀₆, DG1₃₂₆₋₃₄₀, DG1₁₋₁₅, DG1₃₅₋₄₉, DG1₃₂₅₋₃₃₉, or a fragment or an equivalent of each thereof, and the MHC protein of the pMHC complex comprises all or part of HLA-DR or a fragment or an equivalent thereof;
f) a neuromyelitis optica spectrum disorder-relevant antigen derived from an antigen selected from one or more of the group: AQP4₁₂₉₋₁₄₃, AQP4₂₈₄₋₂₉₈, AQP4₆₃₋₇₆, AQP4₁₂₉₋₁₄₃, AQP4₃₉₋₅₃, or a fragment or an equivalent of each thereof, and the MHC protein of the pMHC complex comprises all or part of HLA-DR or a fragment or an equivalent thereof;
g) an allergic asthma-relevant antigen derived from an antigen selected from one or more of the group: DERP1₁₆₋₃₀, DERP1₁₇₁₋₁₈₅, DERP1₁₁₀₋₁₂₄, DERP-2₂₆₋₄₀, DERP-2₁₀₇₋₁₂₁, or a fragment or an equivalent of each thereof, and the MHC protein of the pMHC complex comprises all or part of a polypeptide of the group: HLA-DR, HLA-DP, or a fragment or an equivalent of each thereof;
h) an inflammatory bowel disease-relevant antigen derived from an antigen selected from one or more of the group: bacteroides integrase antigen₁₈₃₋₁₉₇, bacteroides integrase antigen₁₄₆₋₁₆₀, bacteroides integrase antigen₁₇₅₋₁₈₉, bacteroides integrase antigen₁₋₁₅, bacteroides integrase antigen₁₈₃₋₁₉₇, bacteroides integrase antigen₃₀₋₄₄, bacteroides integrase antigen₇₀₋₈₄, bacteroides integrase antigen₃₃₇₋₃₅₁, bacteroides integrase antigen₁₇₁₋₁₈₅, bacteroides integrase antigen₄₋₁₈, bacteroides integrase antigen₂₅₆₋₂₇₀, Fla-2/Fla-X₃₆₆₋₃₈₀, Fla-2/Fla-X₁₆₄₋₁₇₈, Fla-2/Fla-X₂₆₁₋₂₇₅, Fla-2/Fla-X₁₋₁₅, Fla-2/Fla-X₅₁₋₆₅, Fla-2/Fla-X₂₆₉₋₂₈₃, Fla-2/Fla-X₄₋₁₈, Fla-2/Fla-X₂₇₁₋₂₈₅, YIDX₇₈₋₉₂, YIDX₉₃₋₁₀₇, YIDX₉₈₋₁₁₂, YIDX₂₃₋₃₇, YIDX₇₈₋₉₂, YIDX₁₉₅₋₂₀₉, YIDX₂₂₋₃₆, YIDX₈₀₋₉₄, YIDX₁₀₁₋₁₁₅, or a fragment or an equivalent of each thereof, and the MHC protein of the pMHC complex comprises all or part of HLA-DR or a fragment or an equivalent thereof;
i) a systemic lupus erythematosus-relevant antigen derived from an antigen selected from one or more of the group: H4₇₁₋₉₄, H4₇₄₋₈₈, H4₇₆₋₉₀, H4₇₅₋₈₉, H4₇₈₋₉₂, H4₈₀₋₉₄, H2B₁₀₋₂₄, H2B₁₆₋₃₀, H1′₂₂₋₄₂, H1′₂₇₋₄₁, or a fragment or an equivalent of each thereof, and the MHC protein of the pMHC complex comprises all or part of a polypeptide of the group: I-A_{d}, HLA-DR, or a fragment or an equivalent of each thereof;
j) an atherosclerosis-relevant antigen derived from an antigen selected from one or more of the group: ApoB₃₅₀₁₋₃₅₁₆, ApoB₁₉₅₂₋₁₉₆₆, ApoB₉₇₈₋₉₉₃, ApoB₃₄₉₈₋₃₅₁₃, ApoB_{210A}, ApoB_{210B}, ApoB_{210C}, or a fragment or an equivalent of each thereof, and the MHC protein of the pMHC complex comprises all or part of I-A_{b} or a fragment or an equivalent thereof;
k) a COPD-relvant antigen and/or emphysema-relevant antigen, each of which is derived from an antigen selected from one or more of the group: elastin₈₉₋₁₀₃, elastin₆₉₈₋₇₁₂, elastin₈₋₂₂, elastin₉₄₋₁₀₈, elastin₁₃₋₂₇, elastin₆₉₅₋₇₀₉, elastin₅₆₃₋₅₇₇, elastin₅₅₈₋₅₇₂, elastin₆₉₈₋₇₁₂, elastin₅₆₆₋₅₈₀, elastin₅₄₅₋₆₅₉, or a fragment or an equivalent of each thereof, and the MHC protein of the pMHC complex comprises all or part of HLA-DR or a fragment or an equivalent thereof;
l) a psoriasis-relevant antigen derived from an antigen selected from one or more of the group: Cap18₆₄₋₇₈, Cap18₃₄₋₄₈, Cap18₄₇₋₆₁, Cap18₁₅₁₋₁₆₅, Cap18₁₄₉₋₁₆₃, Cap18₁₅₂₋₁₆₆, Cap18₁₃₁₋₁₄₅, Cap18₂₄₋₃₈, ADMTSL5245₋₂₅₉, ADMTSL5₂₆₇₋₂₈₁, ADMTSL5₃₇₂₋₃₈₆, ADMTSL5₂₈₉₋₃₀₃, ADMTSL5₃₉₆₋₄₁₀, ADMTSL5₄₃₃₋₄₄₇, ADMTSL5₁₄₂₋₁₅₆, ADMTSL5₂₃₆₋₂₅₀, ADMTSL5₃₀₁₋₃₁₅, ADMTSL5₂₀₃₋₂₁₇, ADMTSL5₄₀₄₋₄₁₈, or a fragment or an equivalent of each thereof, and the MHC protein of the pMHC complex comprises all or part of HLA-DR or a fragment or an equivalent thereof;
m) an autoimmune hepatitis-relevant antigen derived from an antigen selected from one or more of the group: CYP2D6₁₉₃₋₂₀₇, CYP2D6₇₆₋₉₀, CYP2D6₂₉₃₋₃₀₇, CYP2D6₃₁₃₋₃₃₂, CYP2D6₃₉₃₋₄₁₂, CYP2D6₁₉₉₋₂₁₃, CYP2D6₄₅₀₋₄₆₄, CYP2D6₃₀₁₋₃₁₅, CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₃₁₋₁₄₅, CYP2D6₂₁₆₋₂₃₀, CYP2D6₂₃₈₋₂₅₂, CYP2D6₁₉₉₋₂₁₃, CYP2D6₂₃₅₋₂₅₂, CYP2D6₂₉₃₋₃₀₇, CYP2D6₃₈₁₋₃₉₅, CYP2D6₄₂₉₋₄₄₃, SLA₃₃₄₋₃₄₈, SLA₁₉₆₋₂₁₀, SLA₁₁₅₋₁₂₉, SLA₃₇₃₋₃₈₆, SLA₁₈₆₋₁₉₇, SLA₃₁₇₋₃₃₁, SLA₁₇₁₋₁₈₅, SLA₄₁₇₋₄₃₁, SLA₃₅₉₋₃₇₃, SLA₂₁₅₋₂₂₉, SLA₁₁₁₋₁₂₅, SLA₁₁₀₋₁₂₄, SLA₂₉₉₋₃₁₃, SLA₃₄₂₋₃₅₆, SLA₄₉₋₆₃, SLA₁₁₉₋₁₃₃, SLA₂₆₀₋₂₇₄, SLA₂₆₋₄₀, SLA₈₆₋₁₀₀, SLA₃₃₁₋₃₄₅, or a fragment or an equivalent of each thereof, and the MHC protein of the pMHC complex comprises all or part of HLA-DR or a fragment or an equivalent thereof;
n) an uveitis-relevant antigen derived from an antigen selected from one or more of the group: arrestin₁₉₉₋₂₁₃, arrestin₇₇₋₉₁, arrestin₂₅₀₋₂₆₄, arrestin₁₇₂₋₁₈₆, arrestin₃₅₄₋₃₆₈, arrestin₂₃₉₋₂₅₃, arrestin₁₀₂₋₁₁₆, arrestin₅₉₋₇₃, arrestin₂₈₀₋₂₉₄, arrestin₂₉₁₋₃₀₆, arrestin₁₉₅₋₂₀₉, arrestin₂₀₀₋₂₁₄, or a fragment or an equivalent of each thereof, and the MHC protein of the pMHC complex comprises all or part of HLA-DR or a fragment or an equivalent thereof;
o) a Sjogren's Syndrome-relevant antigen derived from an antigen selected from one or more of the group: RO60₁₂₇₋₁₄₁, RO60₅₂₃₋₅₃₇, RO60₂₄₃₋₂₅₇, RO60₄₈₄₋₄₉₈, RO60₃₄₇₋₃₆₁, RO60₃₆₉₋₃₈₃, RO60₄₂₆₋₄₄₀, RO60₂₆₇₋₂₈₁, RO60₁₇₈₋₁₉₂, RO60₃₅₈₋₃₇₂, RO60₂₂₁₋₂₃₅, RO60₃₁₈₋₃₃₂, RO60₄₀₇₋₄₂₁, RO60₄₅₉₋₄₇₃, RO60₅₁₋₆₅, RO60₃₁₂₋₃₂₆, LA₂₄₁₋₂₅₅, LA₁₀₁₋₁₁₅, LA₁₅₃₋₁₆₇, LA₁₇₈₋₁₉₂, LA₁₉₋₃₃, LA₃₇₋₅₁, LA₁₃₃₋₁₄₇, LA₅₀₋₆₄, LA₃₂₋₄₆, LA₁₅₃₋₁₆₇, LA₈₃₋₉₇, LA₁₃₆₋₁₅₀, LA₂₉₇₋₃₁₁, LA₅₉₋₇₃, LA₁₅₁₋₁₆₅, LA₈₆₋₁₀₀, LA₁₅₄₋₁₆₈, or a fragment or an equivalent of each thereof, and the MHC protein of the pMHC complex comprises all or part of a polypeptide of the group: HLA-DR, HLA-DP, or a fragment or an equivalent of each thereof;
p) a scleroderma-relevant antigen derived from an antigen selected from one or more of the group: TOP1₃₄₆₋₃₆₀, TOP1₄₂₀₋₄₃₄, TOP1₇₅₀₋₇₆₄, TOP1₄₁₉₋₄₃₃, TOP1₅₉₁₋₆₀₅, TOP1₆₉₅₋₇₀₉, TOP1₃₀₅₋₃₁₉, TOP1₃₄₆₋₃₆₀, TOP1₄₁₉₋₄₃₃, TOP1₄₂₅₋₄₃₉, TOP1₆₁₄₋₆₂₈, CENP-C₂₉₇₋₃₁₁, CENP-C₈₅₇₋₈₇₁, CENP-C₈₈₇₋₉₀₁, CENP-C₂₁₂₋₂₂₆, CENP-C₆₄₃₋₆₅₇, CENP-C₈₃₂₋₈₄₆, CENP-C₁₆₇₋₁₈₁, CENP-C₂₄₆₋₂₆₀, CENP-C₈₄₆₋₈₆₀, CENP-C₁₄₉₋₁₆₃, CENP-C₈₃₃₋₈₄₇, CENP-C₈₄₇₋₈₆₁, or a fragment or an equivalent of each thereof, and the MHC protein of the pMHC complex comprises all or part of HLA-DR or a fragment or an equivalent thereof;
q) an anti-phospholipid syndrome-relevant antigen derived from an antigen selected from one or more of the group: APOH₂₃₅₋₂₄₉, APOH₃₀₆₋₃₂₀, APOH₂₃₇₋₂₅₁, APOH₂₉₅₋₃₀₉, APOH₂₈₄₂, APOH₁₇₃₋₁₈₇, APOH₂₆₄₋₂₇₈, APOH₂₉₅₋₃₀₉, APOH₄₉₋₆₃, APOH₂₆₉₋₂₈₃, APOH₂₉₅₋₃₀₉, APOH₃₂₁₋₃₅₅, APOH₃₂₂₋₃₃₆, APOH₃₂₄₋₃₃₈, or a fragment or an equivalent of each thereof, and the MHC protein of the pMHC complex comprises all or part of HLA-DR or a fragment or an equivalent thereof;
r) an ANCA-associated vasculitis-relevant antigen derived from an antigen selected from one or more of the group: MPO₅₀₆₋₅₂₀, MPO₃₀₂₋₃₁₆, MPO₇₋₂₁, MPO₆₈₉₋₇₀₃, MPO₂₄₈₋₂₆₂, MPO₄₄₄₋₄₅₈, MPO₅₁₃₋₅₂₇, MPO₉₇₋₁₁₁, MPO₆₁₆₋₆₃₀, MPO₄₆₂₋₄₇₆, MPO₆₁₇₋₆₃₁, MPO₇₁₄₋₇₂₈, PRTN3₄₄₋₈₅, PRTN3₂₃₄₋₂₄₈, PRTN3₅₉₋₇₃, PRTN3₁₁₇₋₁₃₁, PRTN3₁₆₄₋₁₇₈, PRTN3₇₁₋₈₅, PRTN3₂₄₁₋₂₅₅, PRTN3₅₉₋₇₃, PRTN3₁₈₃₋₁₉₇, PRTN3₆₂₋₇₆, PRTN3₁₁₈₋₁₃₂, PRTN3₂₃₉₋₂₅₃, or a fragment or an equivalent of each thereof, and the MHC protein of the pMHC complex comprises all or part of HLA-DR or a fragment or an equivalent thereof; or
s) a Stiff Man Syndrome-relevant antigen derived from an antigen selected from one or more of the group: GAD₂₁₂₋₂₂₆, GAD₅₅₅₋₅₆₉, GAD₂₉₇₋₃₁₁, and the MHC protein of the pMHC complex comprises all or part of a polypeptide of the group: HLA-DR, HLA-DQ, or a fragment or an equivalent of each thereof.

27. The complex of any of claims 1-26, wherein the pMHC complex is for the treatment of:
a) type I diabetes and the pMHC complex is selected from the group of: PPI_{76-90(K88S)}-HLA-DRB1*0401/DRA, IGRP₁₃₋₂₅-HLA-DRB1*0301/DRA, GAD₅₅₅₋₅₆₇-HLA-DRB1*0401/DRA, GAD_{555-567(557I)}-HLA-DRB1*0401/DRA, IGRP₂₃₋₃₅-HLA-DRB1*0401/DRA, B₂₄-C₃₆-HLA-DRB1*0301/DRA, or PPI₇₆₋₉₀-HLA-DRB1*0401/DRA;
b) multiple sclerosis and the pMHC complex is selected from the group of: MBP₈₆₋₉₈-HLA-DRB1*1501/DRA, MBP₈₉₋₁₀₁-HLA-DRB5*0101/DRA, MOG₃₈₋₅₂-HLA-DRB4*0101/DRA, MOG_{97-109(E107S)}-HLA-DRB1*0401/DRA, MOG₂₀₃₋₂₁₇-HLA-DRB3*0101/DRA, PLP₅₄₋₆₈-HLA-DRB3*0101/DRA, PLP₉₄₋₁₀₈-HLA-DRB1*0301/DRA, PLP₂₅₀₋₂₆₄-HLA-DRB4*0101/DRA, MPB₁₃₋₃₂-HLA-DRB5*0101/DRA, MPB₈₃₋₉₉-HLA-DRB5*0101/DRA, MPB₁₁₁₋₁₂₉-HLA-DRB5*0101/DRA, MPB₁₄₆₋₁₇₀-HLA-DRB5*0101/DRA, MOG₂₂₃₋₂₃₇-HLA-DRB3*0202/DRA, MOG₆₋₂₀-HLA-DRB5*0101/DRA, PLP₈₈₋₁₀₂-HLA-DRB3*0202/DRA, or PLP₁₃₉₋₁₅₄-HLA-DRB5*0101/DRA;
c) Celiac Disease and the pMHC complex is selected from the group of: aGlia₅₇₋₆₈-HLA-DQA1*0501/HLA-DQB1*0201, aGlia₆₂₋₇₂-HLA-DQA1*0501/HLA-DQB1*0201, aGlia₂₁₇₋₂₂₉-HLA-DQA1*0501/HLA-DQB1*0302, or aGlia₂₁₇₋₂₂₉-HLA-DQA1*03/HLA-DQB1*0302;
d) primary biliary cirrhosis and the pMHC complex is selected from the group of: PDC-E2₁₂₂₋₁₃₅-HLA-DRB4*0101/DRA, PDC-E2₂₄₉₋₂₆₂-HLA-DRB4*0101/DRA, PDC-E2₂₄₉₋₂₆₃-HLA-DRB1*0801/DRA, PDC-E2₆₂₉₋₆₄₃-HLA-DRB1*0801/DRA, PDC-E2₇₂₋₈₆-HLA-DRB3*0202/DRA, PDC-E2₃₅₃₋₃₆₇-HLA-DRB3*0202/DRA, PDC-E2₄₂₂₋₄₃₆-HLA-DRB3*0202/DRA, PDC-E2₆₂₉₋₆₄₃-HLA-DRB4*0101/DRA, PDC-E2₈₀₋₉₄-HLA-DRB5*0101/DRA, PDC-E2₅₃₋₃₆₇-HLA-DRB5*0101/DRA, or PDC-E2₅₃₅₋₅₄₉-HLA-DRB5*0101/DRA, mPDC-E2₁₆₆₋₁₅₁-I-A_{g7}, or mPDC-E2₈₂₋₉₆-I-A_{g7};
e) pemphigus folliaceus and/or pemphigus vulgaris and the pMHC complex is selected from the group of: DG1₂₁₆₋₂₂₉-HLA-DRB1*0101/DRA, DG1₂₁₆₋₂₂₉-HLA-DRB1*0102/DRA, DG3₉₇₋₁₁₁-HLA-DRB1*0402/DRA, DG3₂₅₁₋₂₆₅-HLA-DRB1*0402/DRA, DG3₂₅₁₋₂₆₅-HLA-DRB1*0401/DRA, DG3₄₄₁₋₄₅₅-HLA-DRB1*0402/DRA, DG3₃₅₁₋₃₆₅-HLA-DRB3*0202/DRA, DG3₄₅₃₋₄₆₇-HLA-DRB3*0202/DRA, DG3₅₄₀₋₅₅₄-HLA-DRB3*0202/DRA, DG3₂₈₀₋₂₉₄-HLA-DRB4*0101/DRA, DG3₃₂₆₋₃₄₀-HLA-DRB4*0101/DRA, DG3₃₆₇₋₃₈₁-HLA-DRB4*0101/DRA, DG3₁₃₋₂₇-HLA-DRB5*0101/DRA, DG3₃₂₃₋₃₃₇-HLA-DRB5*0101/DRA, DG3₄₃₈₋₄₅₂-HLA-DRB5*0101/DRA, DG1₄₈₋₆₂-HLA-DRB3*0202/DRA, DG1₂₀₆₋₂₂₂-HLA-DRB3*0202/DRA, DG1₃₆₃₋₃₇₇-HLA-DRB3*0202/DRA, DG1₃₋₁₇-HLA-DRB4*0101/DRA, DG1₁₉₂₋₂₀₆-HLA-DRB4*0101/DRA, DG1₃₂₆₋₃₄₀-HLA-DRB4*0101/DRA, DG1₁₋₁₅-HLA-DRB5*0101/DRA, DG1₃₅₋₄₉-HLA-DRB5*0101/DRA, or DG1₃₂₅₋₃₃₉-HLA-DRB5*0101/DRA;
f) neuromyelitis optica spectrum disorder and the pMHC complex is selected from the group of: AQP4₁₂₉₋₁₄₃-HLA-DRB1*0101/DRA, AQP4₂₈₄₋₂₉₈-HLA-DRB1*0301/DRA, AQP4₆₃₋₇₆-HLA-DRB1*0301/DRA, AQP4₁₂₉₋₁₄₃-HLA-DRB1*0401/DRA, or AQP4₃₉₋₅₃-HLA-DRB1*1501/DRA;
g) allergic asthma and the pMHC complex is selected from the group of: DERP-1₁₆₋₃₀-HLA-DRB1*0101/DRA, DERP-1₁₆₋₃₀-HLA-DRB1*1501/DRA, DERP₁₁₇₁₋₁₈₅-HLA-DRB1*1501/DRA, DERP-1₁₁₀₋₁₂₄-HLA-DPB1*0401/DRA, DERP-2₂₆₋₄₀-HLA-DRB1*0101/DRA; DERP-2₂₆₋₄₀-HLA-DRB1*1501/DRA, or DERP-2₁₀₇₋₁₂₁-HLA-DRB1*0301/DRA;
h) inflammatory bowel disease and the pMHC complex is selected from the group of: bacteroides integrase antigen₁₈₃₋₁₉₇-HLA-DRB3*0101/DRA, bacteroides integrase antigen₁₄₆₋₁₆₀-HLA-DRB3*0101/DRA, bacteroides integrase antigen₁₇₅₋₁₈₉-HLA-DRB3*0101/DRA, bacteroides integrase antigen₁₋₁₅-HLA-DRB5*0101/DRA, bacteroides integrase antigen₁₈₃₋₁₉₇-HLA-DRB5*0101/DRA, bacteroides integrase antigen₁₈₃₋₁₉₇-HLA-DRB3*0101/DRA, bacteroides integrase antigen₃₀₋₄₄-HLA-DRB5*0101/DRA, bacteroides integrase antigen₇₀₋₈₄-HLA-DRB4*0101/DRA, bacteroides integrase antigen₃₃₇₋₃₅₁-HLA-DRB4*0101/DRA, bacteroides integrase antigen₁₇₁₋₁₈₅-HLA-DRB4*0101/DRA, bacteroides integrase antigen₄₋₁₈-HLA-DRB3*0202/DRA, bacteroides integrase antigen₁₇₁₋₁₈₅-HLA-DRB3*0202/DRA, bacteroides integrase antigen₂₅₆₋₂₇₀-HLA-DRB3*0202/DRA, Fla-2/Fla-X₃₆₆₋₃₈₀-HLA-DRB3*0101/DRA, Fla-2/Fla-X₁₆₄₋₁₇₈-HLA-DRB3*0101/DRA, Fla-2/Fla-X₂₆₁₋₂₇₅-HLA-DRB5*0101/DRA, Fla-2/Fla-X₁₋₁₅-HLA-DRB5*0101/DRA, Fla-2/Fla-X₅₁₋₆₅-HLA-DRB4*0101/DRA, Fla-2/Fla-X₂₆₉₋₂₈₃-HLA-DRB4*0101/DRA, Fla-2/Fla-X₄-is-HLA-DRB3*0202/DRA, Fla-2/Fla-X₂₆₁₋₂₇₅-HLA-DRB3*0202/DRA, Fla-2/Fla-X₂₇₁₋₂₈₅-HLA-DRB3*0202/DRA, YIDX₇₈₋₉₂-HLA-DRB3*0101/DRA, YIDX₇₈₋₉₂-HLA-DRB4*0101/DRA, YIDX₉₃₋₁₀₇-HLA-DRB3*0101/DRA, YIDX₉₈₋₁₁₂-HLA-DRB5*0101/DRA, YIDX₂₃₋₃₇-HLA-DRB5*0101/DRA, YIDX₇₈₋₉₂-HLA-DRB4*0101/DRA, YIDX₁₉₅₋₂₀₉-HLA-DRB4*0101/DRA, YIDX₂₂₋₃₆-HLA-DRB3*0202/DRA, YIDX₈₀₋₉₄-HLA-DRB3*0202/DRA, or YIDX₁₀₁₋₁₁₅-HLA-DRB3*0202/DRA;
i) COPD and/or emphysema and the pMHC complex is selected from the group of: elastin₈₉₋₁₀₃-HLA-DRB3*0101/DRA, elastin₆₉₈₋₇₁₂-HLA-DRB5*0101/DRA, elastin₈₋₂₂-HLA-DRB5*0101/DRA, elastin₉₄₋₁₀₈-HLA-DRB5*0101/DRA, elastin₁₃₋₂₇-HLA-DRB4*0101/DRA, elastin₆₉₅₋₇₀₉-HLA-DRB4*0101/DRA, elastin₅₆₃₋₅₇₇-HLA-DRB4*0101/DRA, elastin₅₅₈₋₅₇₂-HLA-DRB4*0101/DRA, elastin₆₉₈₋₇₁₂-HLA-DRB5*0101/DRA, elastin₅₆₆₋₅₈₀-HLA-DRB3*0202/DRA, or elastin₅₄₅₋₆₅₉-HLA-DRB3*0202/DRA;
j) psoriasis and the pMHC complex is selected from the group of: Cap18₆₄₋₇₈-HLA-DRB3*0101/DRA, Cap18₃₄₋₄₈-HLA-DRB3*0101/DRA, Cap18₄₇₋₆₁-HLA-DRB3*0101/DRA, Cap18₁₅₁₋₁₆₅-HLA-DRB4*0101/DRA, Cap18₁₄₉₋₁₆₃-HLA-DRB5*0101/DRA, Cap18₁₅₂₋₁₆₆-HLA-DRB5*0101/DRA, Cap18₁₃₁₋₁₄₅-HLA-DRB5*0101/DRA, Cap18₂₄₋₃₈-HLA-DRB3*0202/DRA, ADMTSL5₂₄₅₋₂₅₉-HLA-DRB3*0101/DRA, ADMTSL5₂₆₇₋₂₈₁-HLA-DRB3*0101/DRA, ADMTSL5₃₇₂₋₃₈₆-HLA-DRB3*0101/DRA, ADMTSL5₂₈₉₋₃₀₃-HLA-DRB4*0101/DRA, ADMTSL5₃₉₆₋₄₁₀-HLA-DRB4*0101/DRA, ADMTSL5₄₃₃₋₄₄₇-HLA-DRB4*0101/DRA, ADMTSL5₁₄₂₋₁₅₆-HLA-DRB5*0101/DRA, ADMTSL5₂₃₆₋₂₅₀-HLA-DRB5*0101/DRA, ADMTSL5₃₀₁₋₃₁₅-HLA-DRB5*0101/DRA, ADMTSL5₂₀₃₋₂₁₇-HLA-DRB3*0202/DRA, ADMTSL5₄₀₄₋₄₁₈-HLA-DRB3*0202/DRA, or ADMTSL5₄₃₃₋₄₄₇-HLA-DRB3*0202/DRA;
k) autoimmune hepatitis and the pMHC complex is selected from the group of: CYP2D6₁₉₃₋₂₀₇-HLA-DRB1*0301/DRA, CYP2D6₇₆₋₉₀-HLA-DRB1*0301/DRA, CYP2D6₂₉₃₋₃₀₇-HLA-DRB1*0301/DRA, CYP2D6₃₁₃₋₃₃₂-HLA-DRB1*0301/DRA, CYP2D6₃₉₃₋₄₁₂-HLA-DRB1*0301/DRA, CYP2D6₁₉₉₋₂₁₃-HLA-DRB1*0401/DRA, CYP2D6₄₅₀₋₄₆₄-HLA-DRB1*0401/DRA, CYP2D6₃₀₁₋₃₁₅-HLA-DRB1*0401/DRA, CYP2D6₄₅₂₋₄₆₆-HLA-DRB1*0701/DRA, CYP2D6₅₉₋₇₃-HLA-DRB1*0701/DRA, CYP2D6₁₃₀₋₁₄₄-HLA-DRB1*0701/DRA, CYP2D6₁₉₃₋₂₁₂-HLA-DRB1*0701/DRA, CYP2D6₃₀₅₋₃₂₄-HLA-DRB1*0701/DRA, CYP2D6₁₃₁₋₁₄₅-HLA-DRB3*0202/DRA, CYP2D6₂₁₆₋₂₃₀-HLA-DRB3*0202/DRA, CYP2D6₂₃₈₋₂₅₂-HLA-DRB3*0202/DRA, CYP2D6₁₉₉₋₂₁₃-HLA-DRB4*0101/DRA, CYP2D6₂₃₅₋₂₅₂-HLA-DRB4*0101/DRA, CYP2D6₂₉₃₋₃₀₇-HLA-DRB4*0101/DRA, CYP2D6₂₃₈₋₂₅₂-HLA-DRB5*0101/DRA, CYP2D6₃₈₁₋₃₉₅-HLA-DRB5*0101/DRA, CYP2D6₄₂₉₋₄₄₃-HLA-DRB5*0101/DRA, SLA₃₃₄₋₃₄₈-HLA-DRB1*0301/DRA, SLA₁₉₆₋₂₁₀-HLA-DRB1*0301/DRA, SLA₁₁₅₋₁₂₉-HLA-DRB1*0301/DRA, SLA₃₇₃₋₃₈₆-HLA-DRB1*0301/DRA, SLA₁₈₆₋₁₉₇-HLA-DRB1*0301/DRA, SLA₃₁₇₋₃₃₁-HLA-DRB1*0401/DRA, SLA₁₇₁₋₁₈₅-HLA-DRB1*0401/DRA, SLA₄₁₇₋₄₃₁-HLA-DRB1*0401/DRA, SLA₃₅₉₋₃₇₃-HLA-DRB1*0701/DRA, SLA₂₁₅₋₂₂₉-HLA-DRB1*0701/DRA, SLA₁₁₁₋₁₂₅-HLA-DRB1*0701/DRA, SLA₁₁₀₋₁₂₄-HLA-DRB3*0202/DRA, SLA₂₉₉₋₃₁₃-HLA-DRB3*0202/DRA, SLA₃₄₂₋₃₅₆-HLA-DRB3*0202/DRA, SLA₄₉₋₆₃-HLA-DRB4*0101/DRA, SLA₁₁₉₋₁₃₃-HLA-DRB4*0101/DRA, SLA₂₆₀₋₂₇₄-HLA-DRB4*0101/DRA, SLA₂₆₋₄₀-HLA-DRB5*0101/DRA, SLA₈₆₋₁₀₀-HLA-DRB5*0101/DRA, or SLA₃₃₁₋₃₄₅-HLA-DRB5*0101/DRA;
l) uveitis and the pMHC complex is selected from the group of: arrestin₁₉₉₋₂₁₃-HLA-DRB3*0101/DRA, arrestin₇₇₋₉₁-HLA-DRB3*0101/DRA, arrestin₂₅₀₋₂₆₄-HLA-DRB3*0101/DRA, arrestin₁₇₂₋₁₈₆-HLA-DRB4*0101/DRA, arrestin₃₅₄₋₃₆₈-HLA-DRB4*0101/DRA, arrestin₂₃₉₋₂₅₃-HLA-DRB4*0101/DRA, arrestin₁₀₂₋₁₁₆-HLA-DRB5*0101/DRA, arrestin₅₉₋₇₃-HLA-DRB5*0101, arrestin₂₈₀₋₂₉₄-HLA-DRB5*0101, arrestin₂₉₁₋₃₀₆-HLA-DRB1*0301/DRA, arrestin₁₉₅₋₂₀₉-HLA-DRB3*0202/DRA, arrestin₁₉₉₋₂₁₃-HLA-DRB3*0202/DRA, or arrestin₂₀₀₋₂₁₄-HLA-DRB3*0202/DRA;
m) Sjogren Syndrome and the pMHC complex is selected from the group of: RO60₁₂₇₋₁₄₁-HLA-DRB1*0301/DRA, RO60₅₂₃₋₅₃₇-HLA-DRB1*0301/DRA, RO60₂₄₃₋₂₅₇-HLA-DRB1*0301/DRA, RO60₄₈₄₋₄₉₈-HLA-DRB3*0101/DRA, RO60₃₄₇₋₃₆₁-HLA-DRB3*0101/DRA, RO60₃₆₉₋₃₈₃-HLA-DRB3*0101/DRA, RO60₄₂₆₋₄₄₀-HLA-DRB4*0101/DRA, RO60₂₆₇₋₂₈₁-HLA-DRB4*0101/DRA, RO60₁₇₈₋₁₉₂-HLA-DRB4*0101/DRA, RO60₃₅₈₋₃₇₂-HLA-DRB5*0101/DRA, RO60355-372-HLA-DRB4*0101/DRA, RO60₂₂₁₋₂₃₅-HLA-DRB5*0101/DRA, RO60₂₂₁₋₂₃₅-HLA-DRB4*0101/DRA, RO60₃₁₈₋₃₃₂-HLA-DRB5*0101/DRA, RO60₃₁₈₋₃₃₂-HLA-DRB4*0101/DRA, RO60₄₀₇₋₄₂₁-HLA-DRB4*0101/DRA, RO60₄₀₇₋₄₂₁-HLA-DQA1*0501/HLA-DQB1*0201, RO60₄₅₉₋₄₇₃-HLA-DRB4*0101/DRA, RO60₄₅₉₋₄₇₃-HLA-DQA1*0501/HLA-DQB1*0201, RO60₃₁₈₋₃₃₂-HLA-DQA1*0501/HLA-DQB1*0201, RO60₅₁₋₆₅-HLA-DRB3*0202/DRA, RO60₃₁₂₋₃₂₆-HLA-DRB3*0202/DRA, RO60₃₄₇₋₃₆₁-HLA-DRB3*0202/DRA, LA₂₄₁₋₂₅₅-HLA-DRB1*0301/DRA, LA₁₀₁₋₁₁₅-HLA-DRB1*0301/DRA, LA₁₅₃₋₁₆₇-HLA-DRB1*0301/DRA, LA₁₇₈₋₁₉₂-HLA-DRB3*0101/DRA, LA₁₉₋₃₃-HLA-DRB3*0101/DRA, LA₃₇₋₅₁-HLA-DRB3*0101/DRA, LA₁₃₃₋₁₄₇-HLA-DRB4*0101/DRA, LA₅₀₋₆₄-HLA-DRB4*0101/DRA, LA₃₂₄₆-HLA-DRB4*0101/DRA, LA₁₅₃₋₁₆₇-HLA-DRB5*0101/DRA, LA₈₃₋₉₇-HLA-DRB5*0101/DRA, LA₁₃₆₋₁₅₀-HLA-DRB5*0101/DRA, LA₂₉₇₋₃₁₁-HLA-DQA1*0501/HLA-DQB1*0201, LA₅₉₋₇₃-HLA-DQA1*0501/HLA-DQB1*0201, LA₅₉₋₇₃-HLA-DRB4*0101/DRA, LA₁₅₁₋₁₆₅-HLA-DQA1*0501/HLA-DQB1*0201, LA₁₅₁₋₁₆₅-HLA-DRB4*0101/DRA, LA₂₉₇₋₃₁₁-HLA-DRB4*0101/DRA, LA₅₀₋₆₄-HLA-DRB3*0202/DRA, LA₈₆₋₁₀₀-HLA-DRB3*0202/DRA, or LA₁₅₄₋₁₆₈-HLA-DRB3*0202/DRA;
n) scleroderma and the pMHC complex is selected from the group of: TOP1₃₄₆₋₃₆₀-HLA-DRB3*0101/DRA, TOP1₄₂₀₋₄₃₄-HLA-DRB3*0101/DRA, TOP1₇₅₀₋₇₆₄-HLA-DRB3*0101/DRA, TOP1₄₁₉₋₄₃₃-HLA-DRB4*0101/DRA, TOP1₅₉₁₋₆₀₅-HLA-DRB4*0101/DRA, TOP1₆₉₅₋₇₀₉-HLA-DRB4*0101/DRA, TOP1₃₀₅₋₃₁₉-HLA-DRB5*0101/DRA, TOP1₃₄₆₋₃₆₀-HLA-DRB5*0101/DRA, TOP1₄₁₉₋₄₃₃-HLA-DRB5*0101/DRA, TOP1₄₂₀₋₄₃₄-HLA-DRB3*0202/DRA, TOP1₄₂₅₋₄₃₉-HLA-DRB3*0202/DRA, TOP1₆₁₄₋₆₂₈-HLA-DRB3*0202/DRA, CENP-C₂₉₇₋₃₁₁-HLA-DRB3*0101/DRA, CENP-C₈₅₇₋₈₇₁-HLA-DRB3*0101/DRA, CENP-C₈₈₇₋₉₀₁-HLA-DRB3*0101/DRA, CENP-C₂₁₂₋₂₂₆-HLA-DRB4*0101/DRA, CENP-C₆₄₃₋₆₅₇-HLA-DRB4*0101/DRA, CENP-C₈₃₂₋₈₄₆-HLA-DRB4*0101/DRA, CENP-C₁₆₇₋₁₈₁-HLA-DRB5*0101/DRA, CENP-C₂₄₆₋₂₆₀-HLA-DRB5*0101/DRA, CENP-C₈₄₆₋₈₆₀-HLA-DRB5*0101/DRA, CENP-C₁₄₉₋₁₆₃-HLA-DRB3*0202/DRA, CENP-C₈₃₃₋₈₄₇-HLA-DRB3*0202/DRA, or CENP-C₈₄₇₋₈₆₁-HLA-DRB3*0202/DRA;
o) anti-phospholipid syndrome and the pMHC complex is selected from the group of: APOH₂₃₅₋₂₄₉-HLA-DRB3*0101/DRA, APOH₃₀₆₋₃₂₀-HLA-DRB3*0101/DRA, APOH₂₃₇₋₂₅₁-HLA-DRB3*0101/DRA, APOH₂₉₅₋₃₀₉-HLA-DRB3*0101/DRA, APOH₂₈₋₄₂-HLA-DRB4*0101/DRA, APOH₁₇₃₋₁₈₇-HLA-DRB4*0101/DRA, APOH₂₆₄₋₂₇₈-HLA-DRB4*0101/DRA, APOH₂₉₅₋₃₀₉-HLA-DRB4*0101/DRA, APOH₄₉₋₆₃-HLA-DRB5*0101/DRA, APOH₂₆₉₋₂₈₃-HLA-DRB5*0101/DRA, APOH₂₉₅₋₃₀₉-HLA-DRB5*0101/DRA, APOH₃₂₁₋₃₅₅-HLA-DRB3*0202/DRA, APOH₃₂₂₋₃₃₆-HLA-DRB3*0202/DRA, or APOH₃₂₄₋₃₃₈-HLA-DRB3*0202/DRA;
p) ANCA-associated vasculitis and the pMHC complex is selected from the group of: MPO₅₀₆₋₅₂₀-HLA-DRB3*0101/DRA, MPO₃₀₂₋₃₁₆-HLA-DRB3*0101/DRA, MPO₇₋₂₁-HLA-DRB3*0101/DRA, MPO₆₈₉₋₇₀₃-HLA-DRB4*0101/DRA, MPO₂₄₈₋₂₆₂-HLA-DRB4*0101/DRA, MPO₄₄₄₋₄₅₅-HLA-DRB4*0101/DRA, MPO₅₁₃₋₅₂₇-HLA-DRB5*0101/DRA, MPO₉₇₋₁₁₁-HLA-DRB5*0101/DRA, MPO₆₁₆₋₆₃₀-HLA-DRB5*0101/DRA, MPO₄₆₂₋₄₇₆-HLA-DRB3*0202/DRA, MPO₆₁₇₋₆₃₁-HLA-DRB3*0202/DRA, MPO₇₁₄₋₇₂₈-HLA-DRB3*0202/DRA, PRTN3₄₄₋₅₈-HLA-DRB3*0101/DRA, PRTN3₂₃₄₋₂₄₈-HLA-DRB3*0101/DRA, PRTN3₅₉₋₇₃-HLA DRB3*0101/DRA, PRTN3₅₉₋₇₃-HLA-DRB5*0101/DRA, PRTN3₁₁₇₋₁₃₁-HLA-DRB4*0101/DRA, PRTN3₁₆₄₋₁₇₈-HLA-DRB4*0101/DRA, PRTN3₇₁₋₅₅-HLA-DRB4*0101/DRA, PRTN3₂₄₁₋₂₅₅-HLA-DRB5*0101/DRA, PRTN3₁₈₃₋₁₉₇-HLA-DRB5*0101/DRA, PRTN3₆₂₋₇₆-HLA-DRB3*0202/DRA, PRTN3₁₁₈₋₁₃₂-HLA-DRB3*0202/DRA, or PRTN3₂₃₉₋₂₅₃-HLA-DRB3*0202/DRA; or
q) Stiff Man Syndrome and the pMHC complex is selected from the group of: GAD₂₁₂₋₂₂₆-HLA-DRB1*0801/DRA, GAD₅₅₅₋₅₆₉-HLA-DRB1*0801/DRA, or GAD₂₉₇₋₃₁₁-HLA-DRB1*0301/DRA.

28. The complex of any of claims 1-27, wherein the pMHC complex is for the treatment of:
a) type I diabetes and the pMHC complex is selected from the group of: PPI_{76-90(K88S)}-HLA-DRB1*0401/DRA, IGRP₁₃₋₂₅-HLA-DRB1*0301/DRA, GAD₅₅₅₋₅₆₇ HLA-DRB1*0401/DRA, GAD_{555-567(557I)}-HLA-DRB1*0401/DRA, IGRP₂₃₋₃₅-HLA-DRB1*0401/DRA, or PPI₇₆₋₉₀-HLA-DRB1*0401/DRA;
b) multiple sclerosis and the pMHC complex is selected from the group of: MBP₈₆₋₉₈-HLA-DRB1*1501/DRA, MBP₈₉₋₁₀₁-HLA-DRB5*0101/DRA, MOG₃₈₋₅₂-HLA-DRB4*0101/DRA, MOG_{97-109(E107S)}-HLA-DRB1*0401/DRA, MOG₂₀₃₋₂₁₇-HLA-DRB3*0101/DRA, PLP₅₄₋₆₈-HLA-DRB3*0101/DRA, PLP₉₄₋₁₀₈-HLA-DRB1*0301/DRA, PLP₂₅₀₋₂₆₄-HLA-DRB4*0101/DRA, MPB₁₃₋₃₂-HLA-DRB5*0101/DRA, MPB₈₃₋₉₉-HLA-DRB5*0101/DRA, MPB₁₁₁₋₁₂₉-HLA-DRB5*0101/DRA, MPB₁₄₆₋₁₇₀-HLA-DRB5*0101/DRA, MOG₂₂₃₋₂₃₇-HLA-DRB3*0202/DRA, MOG₆₋₂₀-HLA-DRB5*0101/DRA, PLP₈₈₋₁₀₂-HLA-DRB3*0202/DRA, or PLP₁₃₉₋₁₅₄-HLA-DRB5*0101/DRA;
c) Celiac Disease and the pMHC complex is selected from the group of: aGlia₅₇₋₆₈-HLA-DQA1*0501/HLA-DQB1*0201, aGlia₆₂₋₇₂-HLA-DQA1*0501/HLA-DQB1*0201, or aGlia₂₁₇₋₂₂₉-HLA-DQA1*0501/HLA-DQB1*0302;
d) primary biliary cirrhosis and the pMHC complex is selected from the group of: PDC-E2₁₂₂₋₁₃₅-HLA-DRB4*0101/DRA, PDC-E2₂₄₉₋₂₆₂-HLA-DRB4*0101/DRA, PDC-E2₂₄₉₋₂₆₃-HLA-DRB1*0801/DRA, PDC-E2₆₂₉₋₆₄₃-HLA-DRB1*0801/DRA, PDC-E2₇₂₋₈₆-HLA-DRB3*0202/DRA, PDC-E2₃₅₃₋₃₆₇-HLA-DRB3*0202/DRA, PDC-E2₄₂₂₋₄₃₆-HLA-DRB3*0202/DRA, PDC-E2₆₂₉₋₆₄₃-HLA-DRB4*0101/DRA, PDC-E2₈₀₋₉₄-HLA-DRB5*0101/DRA, PDC-E2₃₅₃₋₃₆₇-HLA-DRB5*0101/DRA, or PDC-E2₅₃₅₋₅₄₉-HLA-DRB5*0101/DRA;
e) pemphigus folliaceus and/or pemphigus vulgaris and the pMHC complex is selected from the group of: DG1₂₁₆₋₂₂₉-HLA-DRB1*0101/DRA, DG3₉₇₋₁₁-HLA-DRB1*0402/DRA, DG3₂₅₁₋₂₆₅-HLA-DRB1*0401/DRA, DG3₄₄₁₋₄₅₅-HLA-DRB1*0402/DRA, DG3₃₅₁₋₃₆₅-HLA-DRB3*0202/DRA, DG3₄₅₃₋₄₆₇-HLA-DRB3*0202/DRA, DG3₅₄₀₋₅₅₄-HLA-DRB3*0202/DRA, DG3₂₈₀₋₂₉₄-HLA-DRB4*0101/DRA, DG3₃₂₆₋₃₄₀-HLA-DRB4*0101/DRA, DG3₃₆₇₋₃₈₁-HLA-DRB4*0101/DRA, DG3₁₃₋₂₇-HLA-DRB5*0101/DRA, DG3₃₂₃₋₃₃₇-HLA-DRB5*0101/DRA, DG3₄₃₈₋₄₅₂-HLA-DRB5*0101/DRA, DG1₄₈₋₆₂-HLA-DRB3*0202/DRA, DG1₂₀₆₋₂₂₂-HLA-DRB3*0202/DRA, DG1₃₆₃₋₃₇₇-HLA-DRB3*0202/DRA, DG1₃₋₁₇-HLA-DRB4*0101/DRA, DG1₁₉₂₋₂₀₆-HLA-DRB4*0101/DRA, DG1₃₂₆₋₃₄₀-HLA-DRB4*0101/DRA, DG1₁₋₁₅-HLA-DRB5*0101/DRA, DG1₃₅₋₄₉-HLA-DRB5*0101/DRA, or DG1₃₂₅₋₃₃₉-HLA-DRB5*0101/DRA;
f) neuromyelitis optica spectrum disorder and the pMHC complex is selected from the group of: AQP4₂₈₄₋₂₉₈-HLA-DRB1*0301/DRA, AQP4₆₃₋₇₆-HLA-DRB1*0301/DRA, AQP4₁₂₉₋₁₄₃-HLA-DRB1*0401/DRA, or AQP4₃₉₋₅₃-HLA-DRB1*1501/DRA;
g) allergic asthma and the pMHC complex is selected from the group of: DERP-1₁₆₋₃₀-HLA-DRB1*0101/DRA, DERP-1₁₆₋₃₀-HLA-DRB1*1501/DRA, DERP₁₁₇₁₋₁₈₅-HLA-DRB1*1501/DRA, DERP-1₁₁₀₋₁₂₄-HLA-DPB1*0401/DRA, DERP-2₂₆₋₄₀-HLA-DRB1*0101/DRA; DERP-2₂₆₋₄₀-HLA-DRB1*1501/DRA, or DERP-2₁₀₇₋₁₂₁-HLA-DRB1*0301/DRA;
h) inflammatory bowel disease and the pMHC complex is selected from the group of: bacteroides integrase antigen₁₋₁₅-HLA-DRB5*0101/DRA, bacteroides integrase antigen₁₈₃₋₁₉₇-HLA-DRB3*0101/DRA, bacteroides integrase antigen₇₀₋₈₄-HLA-DRB4*0101/DRA, bacteroides integrase antigen₄₋₁₈-HLA-DRB3*0202/DRA, bacteroides integrase antigen₁₇₁₋₁₈₅-HLA-DRB3*0202/DRA, bacteroides integrase antigen₂₅₆₋₂₇₀-HLA-DRB3*0202/DRA, Fla-2/Fla-X₃₆₆₋₃₈₀-HLA-DRB3*0101/DRA, Fla-2/Fla-X₂₆₁₋₂₇₅-HLA-DRB5*0101/DRA, Fla-2/Fla-X₅₁₋₆₅-HLA-DRB4*0101/DRA, Fla-2/Fla-X₄₋₁₈-HLA-DRB3*0202/DRA, Fla-2/Fla-X₂₆₁₋₂₇₅-HLA-DRB3*0202/DRA, Fla-2/Fla-X₂₇₁₋₂₈₅-HLA-DRB3*0202/DRA, YIDX₇₈₋₉₂-HLA-DRB3*0101/DRA, YIDX₇₈₋₉₂-HLA-DRB4*0101/DRA, YIDX₉₈₋₁₁₂-HLA-DRB5*0101/DRA, YIDX₂₂₋₃₆-HLA-DRB3*0202/DRA, YIDX₈₀₋₉₄-HLA-DRB3*0202/DRA, or YIDX₁₀₁₋₁₁₅-HLA-DRB3*0202/DRA;
i) emphysema and the pMHC complex is selected from the group of: elastin₈₉₋₁₀₃-HLA-DRB3*0101/DRA, elastin₆₉₈₋₇₁₂-HLA-DRB5*0101/DRA, elastin₅₅₈₋₅₇₂-HLA-DRB4*0101/DRA, elastin₅₆₆₋₅₈₀-HLA-DRB3*0202/DRA, or elastin₅₄₅₋₆₅₉-HLA-DRB3*0202/DRA;
j) psoriasis and the pMHC complex is selected from the group of: Cap18₆₄₋₇₈-HLA-DRB3*0101/DRA, Cap18₃₄₋₄₈-HLA-DRB3*0101/DRA, Cap18₄₇₋₆₁-HLA-DRB3*0101/DRA, Cap18₁₅₁₋₁₆₅-HLA-DRB4*0101/DRA, Cap18₁₄₉₋₁₆₃-HLA-DRB5*0101/DRA, Cap18₁₅₂₋₁₆₆-HLA-DRB5*0101/DRA, Cap18₁₃₁₋₁₄₅-HLA-DRB5*0101/DRA, Cap18₂₄₋₃₈-HLA-DRB3*0202/DRA, ADMTSL5₂₄₅₋₂₅₉-HLA-DRB3*0101/DRA, ADMTSL5₂₆₇₋₂₈₁-HLA-DRB3*0101/DRA, ADMTSL5₃₇₂₋₃₈₆-HLA-DRB3*0101/DRA, ADMTSL5₂₈₉₋₃₀₃-HLA-DRB4*0101/DRA, ADMTSL5₃₉₆₋₄₁₀-HLA-DRB4*0101/DRA, ADMTSL5₄₃₃₋₄₄₇-HLA-DRB4*0101/DRA, ADMTSL5₁₄₂₋₁₅₆-HLA-DRB5*0101/DRA, ADMTSL5₂₃₆₋₂₅₀-HLA-DRB5*0101/DRA, ADMTSL5₃₀₁₋₃₁₅-HLA-DRB5*0101/DRA, ADMTSL5₂₀₃₋₂₁₇-HLA-DRB3*0202/DRA, ADMTSL5₄₀₄₋₄₁₈-HLA-DRB3*0202/DRA, or ADMTSL5₄₃₃₋₄₄₇-HLA-DRB3*0202/DRA;
k) autoimmune hepatitis and the pMHC complex is selected from the group of: CYP2D6₁₉₃₋₂₀₇-HLA-DRB1*0301/DRA, CYP2D6₇₆₋₉₀-HLA-DRB1*0301/DRA, CYP2D6₂₉₃₋₃₀₇-HLA-DRB1*0301/DRA, CYP2D6₃₁₃₋₃₃₂-HLA-DRB1*0301/DRA, CYP2D6₃₉₃₋₄₁₂-HLA-DRB1*0301/DRA, CYP2D6₁₉₉₋₂₁₃-HLA-DRB1*0401/DRA, CYP2D6₄₅₀₋₄₆₄-HLA-DRB1*0401/DRA, CYP2D6₃₀₁₋₃₁₅-HLA-DRB1*0401/DRA, CYP2D6₄₅₂₋₄₆₆-HLA-DRB1*0701/DRA, CYP2D6₅₉₋₇₃-HLA-DRB1*0701/DRA, CYP2D6₁₃₀₋₁₄₄-HLA-DRB1*0701/DRA, CYP2D6₁₉₃₋₂₁₂-HLA-DRB1*0701/DRA, CYP2D6₃₀₅₋₃₂₄-HLA-DRB1*0701/DRA, CYP2D6₁₃₁₋₁₄₅-HLA-DRB3*0202/DRA, CYP2D6₂₁₆₋₂₃₀-HLA-DRB3*0202/DRA, CYP2D6₂₃₈₋₂₅₂-HLA-DRB3*0202/DRA, CYP2D6₁₉₉₋₂₁₃-HLA-DRB4*0101/DRA, CYP2D6₂₃₅₋₂₅₂-HLA-DRB4*0101/DRA, CYP2D6₂₉₃₋₃₀₇-HLA-DRB4*0101/DRA, CYP2D6₂₃₈₋₂₅₂-HLA-DRB5*0101/DRA, CYP2D6₃₈₁₋₃₉₅-HLA-DRB5*0101/DRA, CYP2D6₄₂₉₋₄₄₃-HLA-DRB5*0101/DRA, SLA₃₃₄₋₃₄₈-HLA-DRB1*0301/DRA, SLA₁₉₆₋₂₁₀-HLA-DRB1*0301/DRA, SLA₁₁₅₋₁₂₉-HLA-DRB1*0301/DRA, SLA₃₇₃₋₃₈₆-HLA-DRB1*0301/DRA, SLA₁₈₆₋₁₉₇-HLA-DRB1*0301/DRA, SLA₃₁₇₋₃₃₁-HLA-DRB1*0401/DRA, SLA₁₇₁₋₁₈₅-HLA-DRB1*0401/DRA, SLA₄₁₇₋₄₃₁-HLA-DRB1*0401/DRA, SLA₃₅₉₋₃₇₃-HLA-DRB1*0701/DRA, SLA₂₁₅₋₂₂₉-HLA-DRB1*0701/DRA, SLA₁₁₁₋₁₂₅-HLA-DRB1*0701/DRA, SLA₁₁₀₋₁₂₄-HLA-DRB3*0202/DRA, SLA₂₉₉₋₃₁₃-HLA-DRB3*0202/DRA, SLA₃₄₂₋₃₅₆-HLA-DRB3*0202/DRA, SLA₄₉₋₆₃-HLA-DRB4*0101/DRA, SLA₁₁₉₋₁₃₃-HLA-DRB4*0101/DRA, SLA₂₆₀₋₂₇₄-HLA-DRB4*0101/DRA, SLA₂₆₋₄₀-HLA-DRB5*0101/DRA, SLA₈₆₋₁₀₀-HLA-DRB5*0101/DRA, or SLA₃₃₁₋₃₄₅-HLA-DRB5*0101/DRA;
l) uveitis and the pMHC complex is selected from the group of: arrestin₁₉₉₋₂₁₃-HLA-DRB3*0101/DRA, arrestin₇₇₋₉₁-HLA-DRB3*0101/DRA, arrestin₂₅₀₋₂₆₄-HLA-DRB3*0101/DRA, arrestin₁₇₂₋₁₈₆-HLA-DRB4*0101/DRA, arrestin₃₅₄₋₃₆₈-HLA-DRB4*0101/DRA, arrestin₂₃₉₋₂₅₃-HLA-DRB4*0101/DRA, arrestin₁₀₂₋₁₁₆-HLA-DRB5*0101/DRA, arrestin₅₉₋₇₃-HLA-DRB5*0101, arrestin₂₈₀₋₂₉₄-HLA-DRB5*0101, arrestin₂₉₁₋₃₀₆-HLA-DRB1*0301/DRA, arrestin₁₉₅₋₂₀₉-HLA-DRB3*0202/DRA, arrestin₁₉₉₋₂₁₃-HLA-DRB3*0202/DRA, or arrestin₂₀₀₋₂₁₄-HLA-DRB3*0202/DRA;
m) Sjogren Syndrome and the pMHC complex is selected from the group of: RO60₁₂₇₋₁₄₁-HLA-DRB1*0301/DRA, RO60₅₂₃₋₅₃₇-HLA-DRB1*0301/DRA, RO60₂₄₃₋₂₅₇-HLA-DRB1*0301/DRA, RO60₄₈₄₋₄₉₈-HLA-DRB3*0101/DRA, RO60₃₄₇₋₃₆₁-HLA-DRB3*0101/DRA, RO60₃₆₉₋₃₈₃-HLA-DRB3*0101/DRA, RO60₄₂₆₋₄₄₀-HLA-DRB4*0101/DRA, RO60₂₆₇₋₂₈₁-HLA-DRB4*0101/DRA, RO60₁₇₈₋₁₉₂-HLA-DRB4*0101/DRA, RO60₃₅₈₋₃₇₂-HLA-DRB5*0101/DRA, RO60₂₂₁₋₂₃₅-HLA-DRB5*0101/DRA, RO60₃₁₈₋₃₃₂-HLA-DRB5*0101/DRA, RO60₅₁₋₆₅-HLA-DRB3*0202/DRA, RO60₃₁₂₋₃₂₆-HLA-DRB3*0202/DRA, RO60₃₄₇₋₃₆₁-HLA-DRB3*0202/DRA, LA₂₄₁₋₂₅₅-HLA-DRB1*0301/DRA, LA₁₀₁₋₁₁₅-HLA-DRB1*0301/DRA, LA₁₅₃₋₁₆₇-HLA-DRB1*0301/DRA, LA₁₇₈₋₁₉₂-HLA-DRB3*0101/DRA, LA₁₉₋₃₃-HLA-DRB3*0101/DRA, LA₃₇₋₅₁-HLA-DRB3*0101/DRA, LA₁₃₃₋₁₄₇-HLA-DRB4*0101/DRA, LA₅₀₋₆₄-HLA-DRB4*0101/DRA, LA₃₂₋₄₆-HLA-DRB4*0101/DRA, LA₁₅₃₋₁₆₇-HLA-DRB5*0101/DRA, LA₈₃₋₉₇-HLA-DRB5*0101/DRA, LA₁₃₆₋₁₅₀-HLA-DRB5*0101/DRA, LA₅₀₋₆₄-HLA-DRB3*0202/DRA, LA₈₆₋₁₀₀-HLA-DRB3*0202/DRA, or LA₁₅₄₋₁₆₈-HLA-DRB3*0202/DRA;
n) scleroderma and the pMHC complex is selected from the group of: TOP1₃₄₆₋₃₆₀-HLA-DRB3*0101/DRA, TOP1₄₂₀₋₄₃₄-HLA-DRB3*0101/DRA, TOP1₇₅₀₋₇₆₄-HLA-DRB3*0101/DRA, TOP1₄₁₉₋₄₃₃-HLA-DRB4*0101/DRA, TOP1₅₉₁₋₆₀₅-HLA-DRB4*0101/DRA, TOP1₆₉₅₋₇₀₉-HLA-DRB4*0101/DRA, TOP1₃₀₅₋₃₁₉-HLA-DRB5*0101/DRA, TOP1₃₄₆₋₃₆₀-HLA-DRB5*0101/DRA, TOP1₄₁₉₋₄₃₃-HLA-DRB5*0101/DRA, TOP1₄₂₀₋₄₃₄-HLA-DRB3*0202/DRA, TOP1₄₂₅₋₄₃₉-HLA-DRB3*0202/DRA, TOP1₆₁₄₋₆₂₈-HLA-DRB3*0202/DRA, CENP-C₂₉₇₋₃₁₁-HLA-DRB3*0101/DRA, CENP-C₈₅₇₋₈₇₁-HLA-DRB3*0101, CENP-C₈₈₇₋₉₀₁-HLA-DRB3*0101, CENP-C₂₁₂₋₂₂₆-HLA-DRB4*0101/DRA, CENP-C₆₄₃₋₆₅₇-HLA-DRB4*0101/DRA, CENP-C₈₃₂₋₅₄₆-HLA-DRB4*0101/DRA, CENP-C₁₆₇₋₁₈₁-HLA-DRB5*0101/DRA, CENP-C₂₄₆₋₂₆₀-HLA-DRB5*0101/DRA, CENP-C₈₄₆₋₈₆₀-HLA-DRB5*0101/DRA, CENP-C₁₄₉₋₁₆₃-HLA-DRB3*0202/DRA, CENP-C₈₃₃₋₈₄₇-HLA-DRB3*0202/DRA, or CENP-C₈₄₇₋₈₆₁-HLA-DRB3*0202/DRA;
o) anti-phospholipid syndrome and the pMHC complex is selected from the group of: APOH₂₃₅₋₂₄₉-HLA-DRB3*0101/DRA, APOH₃₀₆₋₃₂₀-HLA-DRB3*0101/DRA, APOH₂₃₇₋₂₅₁-HLA-DRB3*0101/DRA, APOH₂₉₅₋₃₀₉-HLA-DRB3*0101/DRA, APOH₂₈₋₄₂-HLA-DRB4*0101/DRA, APOH₁₇₃₋₁₈₇-HLA-DRB4*0101/DRA, APOH₂₆₄₋₂₇₈-HLA-DRB4*0101/DRA, APOH₂₉₅₋₃₀₉-HLA-DRB4*0101/DRA, APOH₄₉₋₆₃-HLA-DRB5*0101/DRA, APOH₂₆₉₋₂₈₃-HLA-DRB5*0101/DRA, APOH₂₉₅₋₃₀₉-HLA-DRB5*0101/DRA, APOH₃₂₁₋₃₅₅-HLA-DRB3*0202/DRA, APOH₃₂₂₋₃₃₆-HLA-DRB3*0202/DRA, or APOH₃₂₄₋₃₃₈-HLA-DRB3*0202/DRA;
p) ANCA-associated vasculitis and the pMHC complex is selected from the group of: MPO₅₀₆₋₅₂₀-HLA-DRB3*0101/DRA, MPO₃₀₂₋₃₁₆-HLA-DRB3*0101/DRA, MPO₇₋₂₁-HLA-DRB3*0101/DRA, MPO₆₈₉₋₇₀₃-HLA-DRB4*0101/DRA, MPO₂₄₈₋₂₆₂-HLA-DRB4*0101/DRA, MPO₄₄₄₋₄₅₈-HLA-DRB4*0101/DRA, MPO₅₁₃₋₅₂₇-HLA-DRB5*0101/DRA, MPO₉₇₋₁₁₁-HLA-DRB5*0101/DRA, MPO₆₁₆₋₆₃₀-HLA-DRB5*0101/DRA, MPO₄₆₂₋₄₇₆-HLA-DRB3*0202/DRA, MPO₆₁₇₋₆₃₁-HLA-DRB3*0202/DRA, MPO₇₁₄₋₇₂₈-HLA-DRB3*0202/DRA, PRTN3₄₄₋₅₈-HLA-DRB3*0101/DRA, PRTN3₂₃₄₋₂₄₈-HLA-DRB3*0101/DRA, PRTN3₅₉₋₇₃-HLA DRB3*0101/DRA, PRTN3₅₉₋₇₃-HLA-DRB5*0101/DRA, PRTN3₁₁₇₋₁₃₁-HLA-DRB4*0101/DRA, PRTN3₁₆₄₋₁₇₈-HLA-DRB4*0101/DRA, PRTN3₇₁₋₈₅-HLA-DRB4*0101/DRA, PRTN3₂₄₁₋₂₅₅-HLA-DRB5*0101/DRA, PRTN3₁₈₃₋₁₉₇-HLA-DRB5*0101/DRA, PRTN3₆₂₋₇₆-HLA-DRB3*0202/DRA, PRTN3₁₁₈₋₁₃₂-HLA-DRB3*0202/DRA, or PRTN3₂₃₉₋₂₅₃-HLA-DRB3*0202/DRA; or
q) Stiff Man Syndrome and the pMHC complex is selected from the group of: GAD₂₁₂₋₂₂₆-HLA-DRB1*0801/DRA, GAD₅₅₅₋₅₆₉-HLA-DRB1*0801/DRA, or GAD₂₉₇₋₃₁₁-HLA-DRB1*0301/DRA.

29. A composition comprising a plurality of complexes of any of claims 1 to 28 and a carrier, optionally a pharmaceutically acceptable carrier.

30. The composition of claim 29, wherein for the plurality of nanoparticles, one or more of: the nanoparticle cores are the same or different from each other; and/or the diameters of the nanoparticle cores are the same or different from each other; and/or the valency of the pMHC complexes on each nanoparticle core are the same or different from each other; and/or the density of the pMHC complexes on each nanoparticle core are the same or different from each other; and/or the valency of the co-stimulatory molecules on each nanoparticle core are the same or different from each other; and/or the valency of the cytokines on each nanoparticle core are the same or different from each other.

31. The composition of claim 29 or 30, further comprising one or more of a nanoparticle core coupled to one or more cytokines and/or co-stimulatory molecules,
wherein the nanoparticle core has a diameter selected from the group of: from about 1 nm to about 100 nm; from about 1 nm to about 50 nm; from about 1 nm to about 25 nm; from about 5 nm to about 100 nm; from about 5 nm to about 50 nm; or from about 5 nm to about 25 nm; and
optionally wherein the nanoparticle core further comprises an outer layer on the nanoparticle core.

32. A composition comprising a carrier and one or more of a complex of any of claims 1 to 28 and/or the composition of claims 29-31.

33. The composition of claim 32, wherein the carrier is a pharmaceutically acceptable carrier.

34. A method for differentiating an activated T cell or a memory T cell into a IL-10 producing T_{R}1 cell optionally expressing a marker CD49b and/or LAG3, and/or differentiating a B cell into a regulatory B cell, the method comprising contacting the activated T cell or the memory T cell with an effective amount of the complex of any of claims 1-28 or the composition of claims 29-33, wherein the pMHC density of the nanoparticle is from about 0.4 pMHC/100 nm² to about 12 pMHC/100 nm².

35. The method of claim 34, wherein the contacting is in vitro or in vivo.

36. A method for differentiating an activated T cell or a memory T cell into a IL-10 producing T_{R}1 cell optionally expressing a marker CD49b and/or LAG3 and/or differentiating a B cell into a regulatory B cell in a subject in need thereof, comprising administering to the subject an effective amount of the complex of any of claims 1-28 or the composition of claims 29-33, wherein the pMHC density of the nanoparticle is from about 0.4 pMHC/100 nm² to about 12 pMHC/100 nm².

37. A complex comprising a nanoparticle dendrimer core or a polymeric micelle core and a plurality of disease-relevant antigen-MHC (pMHC) complexes complexed to the core; wherein the nanoparticle core has a diameter selected from the group of: from about 1 nm to about 100 nm; from about 1 nm to about 50 nm; from about 1 nm to about 25 nm; from about 5 nm to about 100 nm; from about 5 nm to about 50 nm; or from about 5 nm to about 25 nm; and wherein the nanoparticle dendrimer core or a polymeric micelle core optionally comprises an outer layer on the surface of the core.

38. The complex of claim 37, wherein the valency of the pMHC complexes per nanoparticle dendrimer core or polymeric micelle core is from about 10:1 to about 6000:1.

39. The complex of claim 37 or 38, wherein the nanoparticle has a pMHC density, from about 0.05 pMHC/100 nm² to about 25 pMHC/100 nm² of the surface area of the nanoparticle dendrimer or polymeric micelle.

40. The complex of any of claims 37-39, further comprising a plurality of co-stimulatory molecules and/or a plurality of cytokines coupled to the nanoparticle dendrimer core or polymeric micelle core, wherein the valency of the co-stimulatory molecules per nanoparticle dendrimer core or polymeric micelle core comprises from about 1 to about 6000; and/or the valency of the cytokines per nanoparticle dendrimer core or polymeric micelle core comprises from about 1 to about 6000.

41. The complex of claim 40, wherein the nanoparticle has a pMHC density from about 0.0022 pMHC/100 nm² to about 13.26 pMHC/100 nm² of the surface area of the nanoparticle dendrimer or polymeric micelle.

42. The complex of any of claims 37-41, wherein the MHC protein of the pMHC complexes are selected from classical MHC class I protein, non-classical MHC class I protein, classical MHC class II protein, non-classical MHC class II protein, any loci of HLA DR, HLA DQ, HLA DP, HLA-A, HLA-B, HLA-C, HLA-E, HLA-F, HLA-G, CD1d, or a fragment or a biological equivalent of each thereof.

43. The complex of claim 42, wherein the MHC molecule of the pMHC complexes is an MHC class I (MHC type I) protein.

44. The complex of claim 42, wherein the MHC molecule of the pMHC complexes is an MHC class II (MHC type II) protein.

45. The complex of any of claims 37-44, wherein the disease-relevant antigen of the pMHC complex is selected from the group of: an autoimmune relevant antigen, an immune inflammation-relevant antigen, a cancer-relevant antigen, a tumor-relevant antigen, or an allergic disease-relevant antigen.

46. The complex of claim 45, wherein the immune inflammation-relevant antigen is one or more selected from the group: an asthma-relevant antigen, a diabetes-relevant antigen, a pre-diabetes relevant antigen, a multiple sclerosis-relevant antigen, an allergic asthma-relevant antigen, a primary biliary cirrhosis-relevant antigen, a cirrhosis-relevant antigen, a Neuromyelitis Optica Spectrum Disorder (Devic's disease, NMO)-relevant antigen, an autoimmune encephalitis-relevant antigen, an antigen relevant to autoantibody-mediated neurological syndromes, a Stiff Man syndrome-relevant antigen, a paraneoplastic disease-relevant antigen, antigens relevant to other diseases of the central and peripheral nervous systems, a Pemphigus vulgaris-relevant antigen, inflammatory bowel disease (IBD)-relevant antigen, Crohn's disease-relevant antigen, Ulcerative Colitis-relevant antigen, an arthritis-relevant antigen, a Rheumatoid Arthritis-relevant antigen, a systemic lupus erythematosus (SLE)-relevant antigen, a Celiac Disease relevant antigen, a psoriasis-relevant antigen, an Alopecia Areata-relevant antigen, an Acquired Thrombocytopenic Purpura-relevant antigen, an autoimmune cardiomyopathy-relevant antigen, an idiopathic dilated cardiomyopathy (IDCM)-relevant antigen, a Myasthyenia Gravis-relevant antigen, an Uveitis-relevant antigen, an Ankylosing Spondylitis-relevant antigen, a Grave's Disease-relevant antigen, a Hashimoto's thyroiditis-relevant antigen, an Immune Mediated Myopathies-relevant antigen, an anti-phospholipid syndrome (ANCA+)-relevant antigen, an atherosclerosis-relevant antigen, a scleroderma-relevant antigen, an autoimmune hepatitis-relevant antigen, a dermatomyositis-relevant antigen, a chronic obstructive pulmonary disease-relevant antigen, a spinal cord injury-relevant antigen, a traumatic injury-relevant antigen, a tobacco-induced lung destruction-relevant antigen, a Chronic Obstructive Pulmonary Disease (COPD)-relevant antigen, a lung emphysema-relevant antigen, a sclerosing cholangitis-relevant antigen, a peripheral neuropathy-relevant antigen, a narcolepsy-relevant antigen, a Goodpasture Syndrome-relevant antigen, a Kawasaki's Disease-relevant antigen, an autoimmune uveitis-relevant antigen, a colitis-relevant antigen, an emphysema-relevant antigen, a pemphigus-relevant antigen, a pemphigus folliaceus-relevant antigen, an arthritis-relevant antigen, a Sjogren's Syndrome-relevant antigen, an ANCA-associated vasculitis-relevant antigen, a primary sclerosing cholangitis-relevant antigen, an adipose tissue inflammation/diabetes type II-relevant antigen, or an obesity associated adipose tissue inflammation/insulin resistance-relevant antigen.

47. The complex of any of claims 37-46, wherein the disease-relevant antigen of the pMHC complex is a cancer-relevant antigen or a tumor-relevant antigen.

48. The complex of claim 47, wherein the cancer is a carcinoma, sarcoma, myeloma, leukemia, lymphoma, melanoma, mixed types or a metastases thereof.

49. The complex of any of claims 37-48, wherein the pMHC complex is coupled to the nanoparticle dendrimer core or polymeric micelle core or the outer layer by one or more of covalently, non-covalently, or cross-linked and optionally coupled through a linker.

50. The complex of claim 49, wherein the linker is less than 5 kD in size, that is optionally polyethylene glycol.

51. The complex of claim 49 or 50, wherein the linkers are identical or different from each other.

52. The complex of any of claims 37-51, wherein the nanoparticle dendrimer core or polymeric micelle core is bioabsorbable and/or biodegradable.

53. The complex of any of claims 37-52, wherein the valency of the pMHC complex per nanoparticle dendrimer core or polymeric micelle core is from about 10:1 to about 1000:1.

54. The complex of any of claims 37-53, wherein the density of the nanoparticle comprises from about 0.4 pMHC/100 nm² of surface area of the nanoparticle dendrimer or polymeric micelle core to about 25 pMHC/100 nm² of surface area of the nanoparticle dendrimer or polymeric micelle and/or wherein the MHC protein of the pMHC complex comprises a classical or a non-classical MHC class I protein and/or a classical or non-classical MHC class II protein wherein each optionally is the same or different from each other.

55. The complex of any of claims 37-54, wherein the MHC protein of the pMHC complexes per nanoparticle dendrimer core or polymeric micelle core comprises HLA DR, HLA DQ, HLA DP, HLA-A, HLA-B, HLA-C, HLA-E, HLA-F, HLA-G, CD1d, or a fragment or a biological equivalent of each thereof.

56. The complex of any one of claims 37-55, wherein the dendrimer nanoparticle core comprises a highly branched macromolecule having a tree-like structure growing from a core.

57. The complex of claim 56, wherein the dendrimer nanoparticle core comprises a poly(amidoamine)-based dendrimer or a poly-L-lysine-based dendrimer.

58. The complex of any one of claims 37-55, wherein the polymeric micelle core comprises an amphiphilic block co-polymer assembled into a nano-scaled core-shell structure.

59. The complex of claim 58, wherein the polymeric micelle core comprises a polymeric micelle produced using polyethylene glycol-diastearoylphosphatidylethanolamine block copolymer.

60. The complex of any of claims 37-55, wherein per nanoparticle dendrimer core or polymeric micelle core:
the pMHC complexes are the same or different from each other; and/or the MHC protein of the pMHC complexes are the same or different from each other; and/or the cytokines are the same or different from each other; and/or optionally, the costimulatory molecules are the same or different from each other.

61. The complex of any of claims 37-60, wherein the pMHC density is from about 0.4 pMHC/100 nm² to about 6 pMHC/100 nm² and wherein the nanoparticle core has a diameter of from about 1 nm to about 100 nm.

62. The complex of any of claims 37-46 and 49-61, wherein the disease-relevant antigen is:
a) a diabetes-relevant antigen and is derived from an antigen selected from one or more of the group: preproinsulin (PPI), islet-specific glucose-6-phosphatase (IGRP), glutamate decarboxylase (GAD), islet cell autoantigen-2 (ICA2), insulin, proinsulin, or a fragment or an equivalent of each thereof;
b) a multiple sclerosis-relevant antigen and is derived from an antigen selected from one or more of the group: myelin basic protein, myelin associated glycoprotein, myelin oligodendrocyte protein, proteolipid protein, oligodendrocyte myelin oligoprotein, myelin associated oligodendrocyte basic protein, oligodendrocyte specific protein, heat shock proteins, oligodendrocyte specific proteins, NOGO A, glycoprotein Po, peripheral myelin protein 22, 2′3′-cyclic nucleotide 3′-phosphodiesterase, or a fragment or an equivalent of each thereof;
c) a Celiac Disease-relevant antigen and is derived from gliadin or a fragment or an equivalent thereof;
d) a primary biliary cirrhosis-relevant antigen and is derived from PDC-E2 or a fragment or an equivalent thereof;
e) a pemphigus folliaceus-relevant antigen and/or pemphigus vulgaris-relevant antigen and is derived from an antigen selected from one or more of the group: DG1, DG3, or a fragment or an equivalent of each thereof;
f) a neuromyelitis optica spectrum disorder-relevant antigen and is derived from AQP4 or a fragment or an equivalent thereof;
g) an arthritis-relevant antigen and is derived from an antigen selected from one or more of the group: heat shock proteins, immunoglobulin binding protein, heterogeneous nuclear RNPs, annexin V, calpastatin, type II collagen, glucose-6-phosphate isomerase, elongation factor human cartilage gp39, mannose binding lectin, citrullinated vimentin, type II collagen, fibrinogen, alpha enolase, anti-carbamylated protein (anti-CarP), peptidyl arginine deiminase type 4 (PAD4), BRAF, fibrinogen gamma chain, inter-alpha-trypsin inhibitor heavy chain H1, alpha-1-antitrypsin, plasma protease C1 inhibitor, gelsolin, alpha 1-B glycoprotein, ceruloplasmin, inter-alpha-trypsin inhibitor heavy chain H4, complement factor H, alpha 2 macroglobulin, serum amyloid, C-reactive protein, serum albumin, fibrogen beta chain, serotransferin, alpha 2 HS glycoprotein, vimentin, Complement C3, or a fragment or an equivalent of each thereof;
h) an allergic asthma-relevant antigen and is derived from an antigen selected from one or more of the group: DERP1, DERP2, or a fragment or an equivalent of each thereof;
i) an inflammatory bowel disease-relevant antigen and is derived from an antigen selected from one or more of the group: Flagelin, Fla-2, Fla-X, YIDX, bacteroides integrase, or a fragment or an equivalent of each thereof;
j) a systemic lupus erythematosus-relevant antigen and is derived from an antigen selected from one or more of the group: double-stranded (ds)DNA, ribonucleoprotein (RNP), Smith (Sm), Sjögren's-syndrome-related antigen A (SS-A)/Ro, Sjögren's-syndrome-related antigen B (SS-B)/La, RO60, RO52, histones, or a fragment or an equivalent of each thereof;
k) an atherosclerosis-relevant antigen and is derived from an antigen selected from one or more of the group: ApoB, ApoE or a fragment or an equivalent of each thereof;
l) a COPD-relvant antigen and/or emphysema-relevant antigen and is derived from elastin or a fragment or an equivalent thereof;
m) a psoriasis-relevant antigen and is derived from an antigen selected from one or more of the group: Cap18, ADMTSL5, ATL5, or a fragment or an equivalent of each thereof;
n) an autoimmune hepatitis-relevant antigen and is derived from an antigen selected from one or more of the group: CYP2D6, SLA, or a fragment or an equivalent of each thereof;
o) an uveitis-relevant antigen and is derived from arrestin or a fragment or an equivalent thereof;
p) a Sjogren's Syndrome-relevant antigen and is derived from an antigen selected from one or more of the group: (SS-A)/Ro, (SS-B)/La, MR3, RO60, RO52, or a fragment or an equivalent of each thereof;
q) a scleroderma-relevant antigen and is derived from an antigen selected from one or more of the group: CENP-C, TOP 1, RNA polymerase III, or a fragment or an equivalent of each thereof;
r) an anti-phospholipid syndrome-relevant antigen and is derived from APOH or a fragment or an equivalent thereof;
s) an ANCA-associated vasculitis-relevant antigen and is derived from an antigen selected from one or more of the group: MPO, PRTN3, or a fragment or an equivalent of each thereof; or
t) a Stiff Man Syndrome-relevant antigen and is derived from GAD or a fragment or an equivalent thereof.

63. The complex of any of claims 37-62, wherein the MHC protein of the pMHC complex comprises all or part of a classical MHC class I protein, non-classical MHC class I protein, classical MHC class II protein, non-classical MHC class II protein, MHC dimers (Fc fusions), MHC tetramers, or a polymeric form of a MHC protein, wherein the MHC protein optionally comprises a knob-in-hole based MHC-alpha-Fc/MHC-beta-Fc heterodimer or multimer.

64. The complex of any of claims 37-54 and 56-63, wherein the MHC protein of the pMHC complex comprises all or part of a polypeptide of the group: HLA DR, HLA DQ, HLA DP, HLA-A, HLA-B, HLA-C, HLA-E, HLA-F, HLA-G, CD1d, or a fragment or an equivalent of each thereof.

65. The complex of any of claims 37-64, wherein the MHC protein of the pMHC complex comprises all or part of a polypeptide of the group: HLA-DR, HLA-DQ, HLA-DP, or a fragment or an equivalent of each thereof.

66. The complex of any of claims 37-65, wherein the MHC protein of the pMHC complex comprises all or part of a polypeptide of the group: HLA-DRB1/DRA, HLA-DRB3/DRA, HLA-DRB4/DRA, HLA-DRB5/DRA, HLA-DQA1/HLA-DQB1, HLA-DPB1/HLA-DPA1, or a fragment or an equivalent of each thereof.

67. The complex of any of claims 37-46 and 49-66, wherein the pMHC complex comprises:
a) a diabetes-relevant antigen derived from an antigen selected from one or more of the group: hInsB₁₀₋₁₈, hIGRP₂₂₈₋₂₃₆, hIGRP₂₆₅₋₂₇₃, IGRP₂₀₆₋₂₁₄, hIGRP₂₀₆₋₂₁₄, NRP-A7, NRP-I4, NRP-V7, YAI/D^{b}, INS B₁₅₋₂₃, PPI_{76-90 (K88S)}, IGRP₁₃₋₂₅, GAD₅₅₅₋₅₆₇, GAD_{555-567(557I)}, IGRP₂₃₋₃₅, B₂₄-C₃₆, PPI₇₆₋₉₀, INS-I9, TUM, G6Pase, Pro-insulin_{L2-10}, Pro-insulin_{L3-11}, Pro-insulin_{L6-14}, Pro-insulin_{B5-14}, Pro-insulin_{B10-18}, Pro-insulin_{B14-22}, Pro-insulin_{B15-24}, Pro-insulin_{B17-25}, Pro-insulin_{B18-27}, Pro-insulin_{B20-27}, Pro-insulin_{B21-29}, Pro-insulin_{B25-C1}, Pro-insulin_{B27-C5}, Pro-insulin_{C20-28}, Pro-insulin_{C25-33}, Pro-insulin_{C29-A5}, Pro-insulin_{A1-10}, Pro-insulin_{A2-10}, Pro-insulin_{A12-20}, or a fragment or an equivalent of each thereof;
b) a multiple sclerosis-relevant antigen derived from an antigen selected from one or more of the group: MOG₃₅₋₅₅, MOG₃₆₋₅₅, MAG₂₈₇₋₂₉₅, MAG₅₀₉₋₅₁₇, MAG₅₅₆₋₅₆₄, MBP₁₁₀₋₁₁₈, MOG₁₁₄₋₁₂₂, MOG₁₆₋₁₇₅, MOG₁₇₂₋₁₈₀, MOG₁₇₉₋₁₈₈, MOG₁₈₈₋₁₉₆, MOG₁₈₁₋₁₈₉, MOG₂₀₅₋₂₁₄, PLP₈₀₋₈₈, MAG₂₈₇₋₂₉₅, MAG₅₀₉₋₅₁₇, MAG₅₅₆₋₅₆₄, MOG₉₇₋₁₀₉ MOG_{97-109(E107S)}, MBP₈₉₋₁₀₁, PLP₁₇₅₋₁₉₂, PLP₉₄₋₁₀₈, MBP₈₆₋₉₈, PLP₅₄₋₆₈, PLP₂₄₉₋₂₆₃, MOG₁₅₆₋₁₇₀, MOG₂₀₁₋₂₁₅, MOG₃₈₋₅₂, MOG₂₀₃₋₂₁₇, PLP₂₅₀₋₂₆₄, MPB₁₃₋₃₂, MPB₈₃₋₉₉, MPB₁₁₁₋₁₂₉, MPB₁₄₆₋₁₇₀, MOG₂₂₃₋₂₃₇, MOG₆₋₂₀, PLP₈₈₋₁₀₂, PLP₁₃₉-154, or a fragment or an equivalent of each thereof;
c) a Celiac Disease-relevant antigen derived from an antigen selected from one or more of the group: aGlia₅₇₋₆₈, aGlia₆₂₋₇₂, aGlia₂₁₇₋₂₂₉, or a fragment or an equivalent of each thereof;
d) a primary biliary cirrhosis-relevant antigen derived from an antigen selected from one or more of the group: PDC-E2₁₂₂₋₁₃₅, PDC-E2₂₄₉₋₂₆₂, PDC-E2₂₄₉₋₂₆₃, PDC-E2₆₂₉₋₆₄₃, PDC-E₂₇₂₋₈₆, PDC-E2₃₅₃₋₃₆₇, PDC-E2₄₂₂₋₄₃₆, PDC-E2₆₂₉₋₆₄₃, PDC-E2₈₀₋₉₄, PDC-E2₃₅₃₋₃₆₇, PDC-E₂₅₃₅₋₅₄₉, or a fragment or an equivalent of each thereof;
e) a pemphigus folliaceus-relevant antigen and/or pemphigus vulgaris-relevant antigen, each of which is derived from an antigen selected from one or more of the group: DG1₂₁₆₋₂₂₉, DG3₉₇₋₁₁₁, DG3₂₅₁₋₂₆₅, DG3₄₄₁₋₄₅₅, DG3₃₅₁₋₃₆₅, DG3₄₅₃₋₄₆₇, DG3₅₄₀₋₅₅₄, DG3₂₈₀₋₂₉₄, DG3₃₂₆₋₃₄₀, DG3₃₆₇₋₃₈₁, DG3₁₃₋₂₇, DG3₃₂₃₋₃₃₇, DG3₄₃₈₋₄₅₂, DG1₄₈₋₆₂, DG1₂₀₆₋₂₂₂, DG1₃₆₃₋₃₇₇, DG1₃₋₁₇, DG1₉₂₋₂₀₆, DG1₃₂₆₋₃₄₀, DG1₁₋₁₅, DG1₃₅₋₄₉, DG1₃₂₅₋₃₃₉, or a fragment or an equivalent of each thereof;
f) a neuromyelitis optica spectrum disorder-relevant antigen derived from an antigen selected from one or more of the group: AQP4₁₂₉₋₁₄₃, AQP4₂₈₄₋₂₉₈, AQP4₆₃₋₇₆, AQP4₁₂₉₋₁₄₃, AQP4₃₉₋₅₃, or a fragment or an equivalent of each thereof;
g) an allergic asthma-relevant antigen derived from an antigen selected from one or more of the group: DERP1₁₆₋₃₀, DERP1₁₇₁₋₈₅, DERP1₁₁₀₋₁₂₄, DERP-2₂₆₋₄₀, DERP-2₁₀₇₋₁₂₁, or a fragment or an equivalent of each thereof;
h) an inflammatory bowel disease-relevant antigen derived from an antigen selected from one or more of the group: bacteroides integrase antigen₁₈₃₋₁₉₇, bacteroides integrase antigen₁₄₆₋₁₆₀, bacteroides integrase antigen₁₇₅₋₆₅₉, bacteroides integrase antigen₁₋₁₅, bacteroides integrase antigen₁₈₃₋₁₉₇, bacteroides integrase antigen₃₀₋₄₄, bacteroides integrase antigen₇₇₋₈₄, bacteroides integrase antigen₃₃₇₋₃₅₁, bacteroides integrase antigen₁₇₁₋₁₈₅, bacteroides integrase antigen₄₋₁₈, bacteroides integrase antigen₂₅₆₋₂₇₀, Fla-2/Fla-X₃₆₆₋₃₈₀, Fla-2/Fla-X₁₆₄₋₁₇₈, Fla-2/Fla-X₂₆₁₋₂₇₅, Fla-2/Fla-X₁₋₁₅, Fla-2/Fla-X₅₁₋₆₅, Fla-2/Fla-X₂₆₉₋₂₈₃, Fla-2/Fla-X₄₋₁₈, Fla-2/Fla-X₂₇₁₋₂₈₅, YIDX₇₈₋₉₂, YIDX₉₃₋₁₀₇, YIDX₉₈₋₁₁₂, YIDX₂₃₋₃₇, YIDX₇₈₋₉₂, YIDX₁₉₅₋₂₀₉, YIDX₂₂₋₃₆, YIDX₈₀₋₉₄, YIDX₁₀₁₋₁₁₅, or a fragment or an equivalent of each thereof;
i) a systemic lupus erythematosus-relevant antigen derived from an antigen selected from one or more of the group: H4₇₁₋₉₄, H4₇₄₋₈₈, H4₇₆₋₉₀, H4₇₅₋₈₉, H4₇₈₋₉₂, H4₈₀₋₉₄, H2B₁₀₋₂₄, H2B₁₆₋₃₀, H1′₂₂₋₄₂, H1′₂₇₋₄₁, or a fragment or an equivalent of each thereof;
j) an atherosclerosis-relevant antigen derived from an antigen selected from one or more of the group: ApoB₃₅₀₁₋₃₅₁₆, ApoB₁₉₅₂₋₁₉₆₆, ApoB₉₇₈₋₉₉₃, ApoB₃₄₉₈₋₃₅₁₃, ApoB_{210A}, ApoB_{210B}, ApoB_{210C}, or a fragment or an equivalent of each thereof;
k) a COPD-relvant antigen and/or emphysema-relevant antigen, each of which is derived from an antigen selected from one or more of the group: elastin₈₉₋₁₀₃, elastin₆₉₈₋₇₁₂, elastin₈₋₂₂, elastin₉₄₋₁₀₈, elastin₁₃₋₂₇, elastin₆₉₅₋₇₀₉, elastin₅₆₃₋₅₇₇, elastin₅₅₈₋₅₇₂, elastin₆₉₈₋₇₁₂, elastin₅₆₆₋₅₈₀, elastin₆₄₅₋₆₅₉, or a fragment or an equivalent of each thereof;
l) a psoriasis-relevant antigen derived from an antigen selected from one or more of the group: Cap18₆₄₋₇₈, Cap18₃₄₋₄₈, Cap18₄₇₋₆₁, Cap18₁₅₁₋₁₆₅, Cap18₁₄₉₋₁₆₃, Cap18₁₅₂₋₁₆₆, Cap18₁₃₁₋₁₄₅, Cap18₂₄₋₃₈, ADMTSL5245₋₂₅₉, ADMTSL5₂₆₇₋₂₈₁, ADMTSL5₃₇₂₋₃₈₆, ADMTSL5₂₈₉₋₃₀₃, ADMTSL5₃₉₆₋₄₁₀, ADMTSL5₄₃₃₋₄₄₇, ADMTSL5₁₄₂₋₁₅₆, ADMTSL5₂₃₆₋₂₅₀, ADMTSL5₃₀₁₋₃₁₅, ADMTSL5₂₀₃₋₂₁₇, ADMTSL5₄₀₄₋₄₁₈, or a fragment or an equivalent of each thereof;
m) an autoimmune hepatitis-relevant antigen derived from an antigen selected from one or more of the group: (CYP2D6)₁₉₃₋₂₀₇, CYP2D6₇₆₋₉₀, CYP2D6₂₉₃₋₃₀₇, CYP2D6₃₁₃₋₃₃₂, CYP2D6₃₉₃₋₄₁₂, CYP2D6₁₉₉₋₂₁₃, CYP2D6₄₅₀₋₄₆₄, CYP2D6₃₀₁₋₃₁₅, CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₃₁₋₁₄₅, CYP2D6₂₁₆₋₂₃₀, CYP2D6₂₃₈₋₂₅₂, CYP2D6₁₉₉₋₂₁₃, CYP2D6₂₃₅₋₂₅₂, CYP2D6₂₉₃₋₃₀₇, CYP2D6₃₈₁₋₃₉₅, CYP2D6₄₂₉₋₄₄₃, SLA₃₃₄₋₃₄₈, SLA₁₉₆₋₂₁₀, SLA₁₁₅₋₁₂₉, SLA₃₇₃₋₃₈₆, SLA₁₈₆₋₁₉₇, SLA₃₁₇₋₃₃₁, SLA₁₇₁₋₁₈₅, SLA₄₁₇₋₄₃₁, SLA₃₅₉₋₃₇₃, SLA₂₁₅₋₂₂₉, SLA₁₁₁₋₁₂₅, SLA₁₁₀₋₁₂₄, SLA₂₉₉₋₃₁₃, SLA₃₄₂₋₃₅₆, SLA₄₉₋₆₃, SLA₁₁₉₋₁₃₃, SLA₂₆₀₋₂₇₄, SLA₂₆₋₄₀, SLA₈₆₋₁₀₀, SLA₃₃₁₋₃₄₅, or a fragment or an equivalent of each thereof;
n) an uveitis-relevant antigen derived from an antigen selected from one or more of the group: arrestin₁₉₉₋₂₁₃, arrestin₇₇₋₉₁, arrestin₂₅₀₋₂₆₄, arrestin₁₇₂₋₁₈₆, arrestin₃₅₄₋₃₆₈, arrestin₂₃₉₋₂₅₃, arrestin₁₀₂₋₁₁₆, arrestin₅₉₋₇₃, arrestin₂₈₀₋₂₉₄, arrestin₂₉₁₋₃₀₆, arrestin₁₉₅₋₂₀₉, arrestin₂₀₀₋₂₁₄, or a fragment or an equivalent of each thereof;
o) a Sjogren's Syndrome-relevant antigen derived from an antigen selected from one or more of the group: RO60₁₂₇₋₁₄₁, RO60₅₂₃₋₅₃₇, RO60₂₄₃₋₂₅₇, RO60₄₈₄₋₄₉₈, RO60₃₄₇₋₃₆₁, RO60₃₆₉₋₃₈₃, RO60₄₂₆₋₄₄₀, RO60₂₆₇₋₂₈₁, RO60₁₇₈₋₁₉₂, RO60₃₅₈₋₃₇₂, RO60₂₂₁₋₂₃₅, RO60₃₁₈₋₃₃₂, RO60₄₀₇₋₄₂₁, RO60₄₅₉₋₄₇₃, RO60₅₁₋₆₅, RO60₃₁₂₋₃₂₆, LA₂₄₁₋₂₅₅, LA₁₀₁₋₁₁₅, LA₁₅₃₋₁₆₇, LA₁₇₈₋₁₉₂, LA₁₉₋₃₃, LA₃₇₋₅₁, LA₁₃₃₋₁₄₇, LA₅₀₋₆₄, LA₃₂₋₄₆, LA₁₅₃₋₁₆₇, LA₈₃₋₉₇, LA₁₃₆₋₁₅₀, LA₂₉₇₋₃₁₁, LA₅₉₋₇₃, LA₁₅₁₋₁₆₅, LA₈₆₋₁₀₀, LA₁₅₄₋₁₆₈, or a fragment or an equivalent of each thereof;
p) a scleroderma-relevant antigen derived from an antigen selected from one or more of the group: TOP1₃₄₆₋₃₆₀, TOP1₄₂₀₋₄₃₄, TOP1₇₅₀₋₇₆₄, TOP1₄₁₉₋₄₃₃, TOP1₅₉₁₋₆₀₅, TOP1₆₉₅₋₇₀₉, TOP1₃₀₅₋₃₁₉, TOP1₃₄₆₋₃₆₀, TOP1₄₁₉₋₄₃₃, TOP1₄₂₅₋₄₃₉, TOP1₆₁₄₋₆₂₈, CENP-C₂₉₇₋₃₁₁, CENP-C₈₅₇₋₈₇₁, CENP-C₈₈₇₋₉₀₁, CENP-C₂₁₂₋₂₂₆, CENP-C₆₄₃₋₆₅₇, CENP-C₈₃₂₋₈₄₆, CENP-C₁₆₇₋₁₈₁, CENP-C₂₄₆₋₂₆₀, CENP-C₈₄₆₋₈₆₀, CENP-C₁₄₉₋₁₆₃, CENP-C₈₃₃₋₈₄₇, CENP-C₈₄₇₋₈₆₁, or a fragment or an equivalent of each thereof;
q) an anti-phospholipid syndrome-relevant antigen derived from an antigen selected from one or more of the group: APOH₂₃₅₋₂₄₉, APOH₃₀₆₋₃₂₀, APOH₂₃₇₋₂₅₁, APOH₂₉₅₋₃₀₉, APOH₂₈₄₂, APOH₁₇₃₋₁₈₇, APOH₂₆₄₋₂₇₈, APOH₂₉₅₋₃₀₉, APOH₄₉₋₆₃, APOH₂₆₉₋₂₈₃, APOH₂₉₅₋₃₀₉, APOH₃₂₁₋₃₅₅, APOH₃₂₂₋₃₃₆, APOH₃₂₄₋₃₃₈, or a fragment or an equivalent of each thereof;
r) an ANCA-associated vasculitis-relevant antigen derived from an antigen selected from one or more of the group: MPO₅₀₆₋₅₂₀, MPO₃₀₂₋₃₁₆, MPO₇₋₂₁, MPO₆₈₉₋₇₀₃, MPO₂₄₈₋₂₆₂, MPO₄₄₄₋₄₅₈, MPO₅₁₃₋₅₂₇, MPO₉₇₋₁₁₁, MPO₆₁₆₋₆₃₀, MPO₄₆₂₋₄₇₆, MPO₆₁₇₋₆₃₁, MPO₇₁₄₋₇₂₈, PRTN3₄₄₋₈₅, PRTN3₂₃₄₋₂₄₈, PRTN3₅₉₋₇₃, PRTN3₁₁₇₋₁₃₁, PRTN3₁₆₄₋₁₇₈, PRTN3₇₁₋₈₅, PRTN3₂₄₁₋₂₅₅, PRTN3₅₉₋₇₃, PRTN3₁₈₃₋₁₉₇, PRTN3₆₂₋₇₆, PRTN3₁₁₈₋₁₃₂, PRTN3₂₃₉₋₂₅₃, or a fragment or an equivalent of each thereof; or
s) a Stiff Man Syndrome-relevant antigen derived from an antigen selected from one or more of the group: GAD₂₁₂₋₂₂₆, GAD₅₅₅₋₅₆₉, GAD₂₉₇₋₃₁₁, or a fragment or an equivalent of each thereof.

68. The complex of any of claims 37-46 and 48-67, wherein the pMHC complex comprises:
a) a diabetes-relevant antigen derived from an antigen selected from one or more of the group: hInsB₁₀₋₁₈, hIGRP₂₂₈₋₂₃₆, hIGRP₂₆₅₋₂₇₃, IGRP₂₀₆₋₂₁₄, hIGRP₂₀₆₋₂₁₄, NRP-A7, NRP-I4, NRP-V7, YAI/D^{b}, INS B₁₅₋₂₃, PPI_{76-90 (K88S)}, IGRP₁₃₋₂₅, GAD₅₅₅₋₅₆₇, GAD_{555-567(557I)}, IGRP₂₃₋₃₅, B₂₄-C₃₆, PPI₇₆₋₉₀, INS-I9, TUM, G6Pase, Pro-insulin_{L2-10}, Pro-insulin_{L3-11}, Pro-insulin_{L6-14}, Pro-insulin_{B5-14}, Pro-insulin_{B10-18}, Pro-insulin_{B14-22}, Pro-insulin_{B15-24}, Pro-insulin_{B17-25}, Pro-insulin_{B18-27}, Pro-insulin_{B20-27}, Pro-insulin_{B21-29}, Pro-insulin_{B25-C1}, Pro-insulin_{B27-C5}, Pro-insulin_{C20-28}, Pro-insulin_{C25-33}, Pro-insulin_{C29-A5}, Pro-insulin_{A1-10}, Pro-insulin_{A2-10}, Pro-insulin_{A12-20}, or a fragment or an equivalent of each thereof, and the MHC protein of the pMHC complex comprises all or part of HLA-DR or a fragment or an equivalent thereof;
b) a multiple sclerosis-relevant antigen derived from an antigen selected from one or more of the group: MOG₃₅₋₅₅, MOG₃₆₋₅₅, MAG₂₈₇₋₂₉₅, MAG₅₀₉₋₅₁₇, MAG₅₅₆₋₅₆₄, MBP₁₁₀₋₁₁₈, MOG₁₁₄₋₁₂₂, MOG₁₆₆₋₁₇₅, MOG₁₇₂₋₁₈₀, MOG₁₇₉₋₁₈₈, MOG₁₈₈₋₁₉₆, MOG₁₈₁₋₁₈₉, MOG₂₀₅₋₂₁₄, PLP₈₀₋₈₈, MAG₂₈₇₋₂₉₅, MAG₅₀₉₋₅₁₇, MAG₅₅₆₋₅₆₄, MOG₉₇₋₁₀₉ MOG_{97-109(E107S)}, MBP₈₉₋₁₀₁, PLP₁₇₅₋₁₉₂, PLP₉₄₋₁₀₈, MBP₈₆₋₉₈, PLP₅₄₋₆₈, PLP₂₄₉₋₂₆₃, MOG₁₅₆₋₁₇₀, MOG₂₀₁₋₂₁₅, MOG₃₈₋₅₂, MOG₂₀₃₋₂₁₇, PLP₂₅₀₋₂₆₄, MPB₁₃₋₃₂, MPB₈₃₋₉₉, MPB₁₁₁₋₁₂₉, MPB₁₄₆₋₁₇₀, MOG₂₂₃₋₂₃₇, MOG₆₋₂₀, PLP₈₈₋₁₀₂, PLP₁₃₉₋₁₅₄, or a fragment or an equivalent of each thereof, and the MHC protein of the pMHC complex comprises all or part of HLA-DR or a fragment or an equivalent thereof;
c) a Celiac Disease-relevant antigen derived from an antigen selected from one or more of the group: aGlia₅₇₋₆₈, aGlia₆₂₋₇₂, aGlia₂₁₇₋₂₂₉, or a fragment or an equivalent of each thereof, and the MHC protein of the pMHC complex comprises all or part of HLA-DQ or a fragment or an equivalent thereof;
d) a primary biliary cirrhosis-relevant antigen derived from an antigen selected from one or more of the group: PDC-E2₁₂₂₋₁₃₅, PDC-E2₂₄₉₋₂₆₂, PDC-E2₂₄₉₋₂₆₃, PDC-E2₆₂₉₋₆₄₃, PDC-E₂₇₂₋₈₆, PDC-E2₃₅₃₋₃₆₇, PDC-E2₄₂₂₋₄₃₆, PDC-E2₆₂₉₋₆₄₃, PDC-E2₈₀₋₉₄, PDC-E2₃₅₃₋₃₆₇, PDC-E₂₅₃₅₋₅₄₉, or a fragment or an equivalent of each thereof, and the MHC protein of the pMHC complex comprises all or part of HLA-DR or a fragment of an equivalent thereof;
e) a pemphigus folliaceus-relevant antigen and/or pemphigus vulgaris-relevant antigen, each of which is derived from an antigen selected from one or more of the group: DG1₂₁₆₋₂₂₉, DG3₉₇₋₁₁₁, DG3₂₅₁₋₂₆₅, DG3₄₄₁₋₄₅₅, DG3₃₅₁₋₃₆₅, DG3₄₅₃₋₄₆₇, DG3₅₄₀₋₅₅₄, DG3₂₈₀₋₂₉₄, DG3₃₂₆₋₃₄₀, DG3₃₆₇₋₃₈₁, DG3₁₃₋₂₇, DG3₃₂₃₋₃₃₇, DG3₄₃₈₋₄₅₂, DG1₄₈₋₆₂, DG1₂₀₆₋₂₂₂, DG1₃₆₃₋₃₇₇, DG1₃₋₁₇, DG1₁₉₂₋₂₀₆, DG1₃₂₆₋₃₄₀, DG1₁₋₁₅, DG1₃₅₋₄₉, DG1₃₂₅₋₃₃₉, or a fragment or an equivalent of each thereof, and the MHC protein of the pMHC complex comprises all or part of HLA-DR or a fragment or an equivalent thereof;
f) a neuromyelitis optica spectrum disorder-relevant antigen derived from an antigen selected from one or more of the group: AQP4₁₂₉₋₁₄₃, AQP4₂₈₄₋₂₉₈, AQP4₆₃₋₇₆, AQP4₁₂₉₋₁₄₃, AQP4₃₉₋₅₃, or a fragment or an equivalent of each thereof, and the MHC protein of the pMHC complex comprises all or part of HLA-DR or a fragment or an equivalent thereof;
g) an allergic asthma-relevant antigen derived from an antigen selected from one or more of the group: DERP1₁₆₋₃₀, DERP1₁₇₁₋₁₈₅, DERP1₁₁₀₋₁₂₄, DERP-2₂₆₋₄₀, DERP-2₁₀₇₋₁₂₁, or a fragment or an equivalent of each thereof, and the MHC protein of the pMHC complex comprises all or part of a polypeptide of the group: HLA-DR, HLA-DP, or a fragment or an equivalent of each thereof;
h) an inflammatory bowel disease-relevant antigen derived from an antigen selected from one or more of the group: bacteroides integrase antigen₁₈₃₋₁₉₇, bacteroides integrase antigen₁₄₋₁₆₀, bacteroides integrase antigen₁₇₅₋₁₈₉, bacteroides integrase antigen₁₋₁₅, bacteroides integrase antigen₁₈₃₋₁₉₇, bacteroides integrase antigen₃₀₋₄₄, bacteroides integrase antigen₇₀₋₈₄, bacteroides integrase antigen₃₃₇₋₃₅₁, bacteroides integrase antigen₁₇₁₋₁₈₅, bacteroides integrase antigen₄₋₁₈, bacteroides integrase antigen₂₅₆₋₂₇₀, Fla-2/Fla-X₃₆₆₋₃₈₀, Fla-2/Fla-X₁₆₄₋₁₇₈, Fla-2/Fla-X₂₆₁₋₂₇₅, Fla-2/Fla-X₁₋₁₅, Fla-2/Fla-X₅₁₋₆s, Fla-2/Fla-X₂₆₉₋₂₈₃, Fla-2/Fla-X₄₋₁₈, Fla-2/Fla-X₂₇₁₋₂₈₅, YIDX₇₈₋₉₂, YIDX₉₃₋₁₀₇, YIDX₉₈₋₁₁₂, YIDX₂₃₋₃₇, YIDX₇₈₋₉₂, YIDX₁₉₅₋₂₀₉, YIDX₂₂₋₃₆, YIDX₈₀₋₉₄, YIDX₁₀₁₋₁₁₅, or a fragment or an equivalent of each thereof, and the MHC protein of the pMHC complex comprises all or part of HLA-DR or a fragment or an equivalent thereof;
i) a systemic lupus erythematosus-relevant antigen derived from an antigen selected from one or more of the group: H4₇₁₋₉₄, H4₇₄₋₈₈, H4₇₆₋₉₀, H4₇₅₋₈₉, H4₇₈₋₉₂, H4₈₀₋₉₄, H2B₁₀₋₂₄, H2B₁₆₋₃₀, H1′₂₂₋₄₂, H1′₂₇₋₄₁, or a fragment or an equivalent of each thereof, and the MHC protein of the pMHC complex comprises all or part of a polypeptide of the group: I-A_{d}, HLA-DR, or a fragment or an equivalent of each thereof;
j) an atherosclerosis-relevant antigen derived from an antigen selected from one or more of the group: ApoB₃₅₀₁₋₃₅₁₆, ApoB₁₉₅₂₋₁₉₆₆, ApoB₉₇₈₋₉₉₃, ApoB₃₄₉₈₋₃₅₁₃, ApoB_{210A}, ApoB_{210B}, ApoB_{210C}, or a fragment or an equivalent of each thereof, and the MHC protein of the pMHC complex comprises all or part of I-A_{b} or a fragment or an equivalent thereof;
k) a COPD-relvant antigen and/or emphysema-relevant antigen, each of which is derived from an antigen selected from one or more of the group: elastin₈₉₋₁₀₃, elastin₆₉₈₋₇₁₂, elastin₈₋₂₂, elastin₉₄₋₁₀₈, elastin₁₃₋₂₇, elastin₆₉₅₋₇₀₉, elastin₅₆₃₋₅₇₇, elastin₅₅₈₋₅₇₂, elastin₆₉₈₋₇₁₂, elastin₅₆₆₋₅₈₀, elastin₅₄₅₋₆₅₉, or a fragment or an equivalent of each thereof, and the MHC protein of the pMHC complex comprises all or part of HLA-DR or a fragment or an equivalent thereof;
l) a psoriasis-relevant antigen derived from an antigen selected from one or more of the group: Cap18₆₄₋₇₈, Cap18₃₄₋₄₈, Cap18₄₇₋₆₁, Cap18₁₅₁₋₁₆₅, Cap18₁₄₉₋₁₆₃, Cap18₁₅₂₋₁₆₆, Cap18₁₃₁₋₁₄₅, Cap18₂₄₋₃₈, ADMTSL5245₋₂₅₉, ADMTSL5₂₆₇₋₂₈₁, ADMTSL5₃₇₂₋₃₈₆, ADMTSL5₂₈₉₋₃₀₃, ADMTSL5₃₉₆₋₄₁₀, ADMTSL5₄₃₃₋₄₄₇, ADMTSL5₁₄₂₋₁₅₆, ADMTSL5₂₃₆₋₂₅₀, ADMTSL5₃₀₁₋₃₁₅, ADMTSL5₂₀₃₋₂₁₇, ADMTSL5₄₀₄₋₄₁₈, or a fragment or an equivalent of each thereof, and the MHC protein of the pMHC complex comprises all or part of HLA-DR or a fragment or an equivalent thereof;
m) an autoimmune hepatitis-relevant antigen derived from an antigen selected from one or more of the group: CYP2D6₁₉₃₋₂₀₇, CYP2D6₇₆₋₉₀, CYP2D6₂₉₃₋₃₀₇, CYP2D6₃₁₃₋₃₃₂, CYP2D6₃₉₃₋₄₁₂, CYP2D6₁₉₉₋₂₁₃, CYP2D6₄₅₀₋₄₆₄, CYP2D6₃₀₁₋₃₁₅, CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₃₁₋₁₄₅, CYP2D6₂₁₆₋₂₃₀, CYP2D6₂₃₈₋₂₅₂, CYP2D6₁₉₉₋₂₁₃, CYP2D6₂₃₅₋₂₅₂, CYP2D6₂₉₃₋₃₀₇, CYP2D6₃₈₁₋₃₉₅, CYP2D6₄₂₉₋₄₄₃, SLA₃₃₄₋₃₄₈, SLA₁₉₆₋₂₁₀, SLA₁₁₅₋₁₂₉, SLA₃₇₃₋₃₈₆, SLA₁₈₆₋₁₉₇, SLA₃₁₇₋₃₃₁, SLA₁₇₁₋₁₈₅, SLA₄₁₇₋₄₃₁, SLA₃₅₉₋₃₇₃, SLA₂₁₅₋₂₂₉, SLA₁₁₁₋₁₂₅, SLA₁₁₀₋₁₂₄, SLA₂₉₉₋₃₁₃, SLA₃₄₂₋₃₅₆, SLA₄₉₋₆₃, SLA₁₁₉₋₁₃₃, SLA₂₆₀₋₂₇₄, SLA₂₆₋₄₀, SLA₈₆₋₁₀₀, SLA₃₃₁₋₃₄₅, or a fragment or an equivalent of each thereof, and the MHC protein of the pMHC complex comprises all or part of HLA-DR or a fragment or an equivalent thereof;
n) an uveitis-relevant antigen derived from an antigen selected from one or more of the group: arrestin₁₉₉₋₂₁₃, arrestin₇₇₋₉₁, arrestin₂₅₀₋₂₆₄, arrestin₁₇₂₋₁₈₆, arrestin₃₅₄₋₃₆₈, arrestin₂₃₉₋₂₅₃, arrestin₁₀₂₋₁₁₆, arrestin₅₉₋₇₃, arrestin₂₈₀₋₂₉₄, arrestin₂₉₁₋₃₀₆, arrestin₁₉₅₋₂₀₉, arrestin₂₀₀₋₂₁₄, or a fragment or an equivalent of each thereof, and the MHC protein of the pMHC complex comprises all or part of HLA-DR or a fragment or an equivalent thereof;
o) a Sjogren's Syndrome-relevant antigen derived from an antigen selected from one or more of the group: RO60₁₂₇₋₁₄₁, RO60₅₂₃₋₅₃₇, RO60₂₄₃₋₂₅₇, RO60₄₈₄₋₄₉₈, RO60₃₄₇₋₃₆₁, RO60₃₆₉₋₃₈₃, RO60₄₂₆₋₄₄₀, RO60₂₆₇₋₂₈₁, RO60₁₇₈₋₁₉₂, RO60₃₅₈₋₃₇₂, RO60₂₂₁₋₂₃₅, RO60₃₁₈₋₃₃₂, RO60₄₀₇₋₄₂₁, RO60₄₅₉₋₄₇₃, RO60₅₁₋₆₅, RO60₃₁₂₋₃₂₆, LA₂₄₁₋₂₅₅, LA₁₀₁₋₁₁₅, LA₁₅₃₋₁₆₇, LA₁₇₈₋₁₉₂, LA₁₉₋₃₃, LA₃₇₋₅₁, LA₁₃₃₋₁₄₇, LA₅₀₋₆₄, LA₃₂₋₄₆, LA₁₅₃₋₁₆₇, LA₈₃₋₉₇, LA₁₃₆₋₁₅₀, LA₂₉₇₋₃₁₁, LA₅₉₋₇₃, LA₁₅₁₋₁₆₅, LA₈₆₋₁₀₀, LA₁₅₄₋₁₆₈, or a fragment or an equivalent of each thereof, and the MHC protein of the pMHC complex comprises all or part of a polypeptide of the group: HLA-DR, HLA-DP, or a fragment or an equivalent of each thereof;
p) a scleroderma-relevant antigen derived from an antigen selected from one or more of the group: TOP1₃₄₆₋₃₆₀, TOP1₄₂₀₋₄₃₄, TOP1₇₅₀₋₇₆₄, TOP1₄₁₉₋₄₃₃, TOP1₅₉₁₋₆₀₅, TOP1₆₉₅₋₇₀₉, TOP1₃₀₅₋₃₁₉, TOP1₃₄₆₋₃₆₀, TOP1₄₁₉₋₄₃₃, TOP1₄₂₅₋₄₃₉, TOP1₆₁₄₋₆₂₈, CENP-C₂₉₇₋₃₁₁, CENP-C₈₅₇₋₈₇₁, CENP-C₈₈₇₋₉₀₁, CENP-C₂₁₂₋₂₂₆, CENP-C₆₄₃₋₈₅₇, CENP-C₈₃₂₋₈₄₆, CENP-C₁₆₇₋₁₈₁, CENP-C₂₄₆₋₂₆₀, CENP-C₈₄₆₋₈₆₀, CENP-C₁₄₉₋₁₆₃, CENP-C₈₃₃₋₈₄₇, CENP-C₈₄₇₋₈₆₁, or a fragment or an equivalent of each thereof, and the MHC protein of the pMHC complex comprises all or part of HLA-DR or a fragment or an equivalent thereof;
q) an anti-phospholipid syndrome-relevant antigen derived from an antigen selected from one or more of the group: APOH₂₃₅₋₂₄₉, APOH₃₀₆₋₃₂₀, APOH₂₃₇₋₂₅₁, APOH₂₉₅₋₃₀₉, APOH₂₈₄₂, APOH₁₇₃₋₁₈₇, APOH₂₆₄₋₂₇₈, APOH₂₉₅₋₃₀₉, APOH₄₉₋₆₃, APOH₂₆₉₋₂₈₃, APOH₂₉₅₋₃₀₉, APOH₃₂₁₋₃₅₅, APOH₃₂₂₋₃₃₆, APOH₃₂₄₋₃₃₈, or a fragment or an equivalent of each thereof, and the MHC protein of the pMHC complex comprises all or part of HLA-DR or a fragment or an equivalent thereof;
r) an ANCA-associated vasculitis-relevant antigen derived from an antigen selected from one or more of the group: MPO₅₀₆₋₅₂₀, MPO₃₀₂₋₃₁₆, MPO₇₋₂₁, MPO₆₈₉₋₇₀₃, MPO₂₄₈₋₂₆₂, MPO₄₄₄₋₄₅₈, MPO₅₁₃₋₅₂₇, MPO₉₇₋₁₁₁, MPO₆₁₆₋₆₃₀, MPO₄₆₂₋₄₇₆, MPO₆₁₇₋₆₃₁, MPO₇₁₄₋₇₂₈, PRTN3₄₄₋₅₈, PRTN3₂₃₄₋₂₄₈, PRTN3₅₉₋₇₃, PRTN3₁₁₇₋₁₃₁, PRTN3₁₆₄₋₁₇₈, PRTN3₇₁₋₈₅, PRTN3₂₄₁₋₂₅₅, PRTN3₅₉₋₇₃, PRTN3₁₈₃₋₁₉₇, PRTN3₆₂₋₇₆, PRTN3₁₁₈₋₁₃₂, PRTN3₂₃₉₋₂₅₃, or a fragment or an equivalent of each thereof, and the MHC protein of the pMHC complex comprises all or part of HLA-DR or a fragment or an equivalent thereof; or
s) a Stiff Man Syndrome-relevant antigen derived from an antigen selected from one or more of the group: GAD₂₁₂₋₂₂₆, GAD₅₅₅₋₅₆₉, GAD₂₉₇₋₃₁₁, and the MHC protein of the pMHC complex comprises all or part of a polypeptide of the group: HLA-DR, HLA-DQ, or a fragment or an equivalent of each thereof.

69. The complex of any of claims 37-46 and 49-68, wherein the pMHC complex is for the treatment of:
a) type I diabetes and the pMHC complex is selected from the group of: PPI_{76-90(K88S)}-HLA-DRB1*0401/DRA, IGRP₁₃₋₂₅-HLA-DRB1*0301/DRA, GAD₅₅₅₋₅₆₇ HLA-DRB1*0401/DRA, GAD_{555-567(557I)}-HLA-DRB1*0401/DRA, IGRP₂₃₋₃₅-HLA-DRB1*0401/DRA, B₂₄-C₃₆-HLA-DRB1*0301/DRA, or PPI₇₆₋₉₀-HLA-DRB1*0401/DRA;
b) multiple sclerosis and the pMHC complex is selected from the group of: MBP₈₅₆₋₉₈-HLA-DRB1*1501/DRA, MBP₈₉₋₁₀₁-HLA-DRB5*0101/DRA, MOG₃₈₋₅₂-HLA-DRB4*0101/DRA, MOG_{97-109(E107S)}-HLA-DRB1*0401/DRA, MOG₂₀₃₋₂₁₇-HLA-DRB3*0101/DRA, PLP₅₄₋₆₈-HLA-DRB3*0101/DRA, PLP₉₄₋₁₀₈-HLA-DRB1*0301/DRA, PLP₂₅₀₋₂₆₄-HLA-DRB4*0101/DRA, MPB₁₃₋₃₂-HLA-DRB5*0101/DRA, MPB₈₃₋₉₉-HLA-DRB5*0101/DRA, MPB₁₁₁₋₁₂₉-HLA-DRB5*0101/DRA, MPB₁₄₆₋₁₇₀-HLA-DRB5*0101/DRA, MOG₂₂₃₋₂₃₇-HLA-DRB3*0202/DRA, MOG₆₋₂₀-HLA-DRB5*0101/DRA, PLP₈₈₋₁₀₂-HLA-DRB3*0202/DRA, or PLP₁₃₉₋₁₅₄-HLA-DRB5*0101/DRA;
c) Celiac Disease and the pMHC complex is selected from the group of: aGlia₅₇₋₆₈-HLA-DQA1*0501/HLA-DQB1*0201, aGlia₆₂₋₇₂-HLA-DQA1*0501/HLA-DQB1*0201, aGlia₂₁₇₋₂₂₉-HLA-DQA1*0501/HLA-DQB1*0302, or aGlia₂₁₇₋₂₂₉-HLA-DQA1*03/HLA-DQB1*0302;
d) primary biliary cirrhosis and the pMHC complex is selected from the group of: PDC-E2₁₂₂₋₁₃₅-HLA-DRB4*0101/DRA, PDC-E2₂₄₉₋₂₆₂-HLA-DRB4*0101/DRA, PDC-E2₂₄₉₋₂₆₃-HLA-DRB1*0801/DRA, PDC-E2₆₂₉₋₆₄₃-HLA-DRB1*0801/DRA, PDC-E2₇₂₋₈₆-HLA-DRB3*0202/DRA, PDC-E2₃₅₃₋₃₆₇-HLA-DRB3*0202/DRA, PDC-E2₄₂₂₋₄₃₆-HLA-DRB3*0202/DRA, PDC-E2₆₂₉₋₆₄₃-HLA-DRB4*0101/DRA, PDC-E2₈₀₋₉₄-HLA-DRB5*0101/DRA, PDC-E2₃₅₃₋₃₆₇-HLA-DRB5*0101/DRA, or PDC-E2₅₃₅₋₅₄₉-HLA-DRB5*0101/DRA, mPDC-E2₁₆₆₋₅₁₁-I-A_{g7}, or mPDC-E2₈₂₋₉₆-I-A_{g7};
e) pemphigus folliaceus and/or pemphigus vulgaris and the pMHC complex is selected from the group of: DG1₂₁₆₋₂₂₉-HLA-DRB1*0101/DRA, DG1₂₁₆₋₂₂₉-HLA-DRB1*0102/DRA, DG3₉₇₋₁₁₁-HLA-DRB1*0402/DRA, DG3₂₅₁₋₂₆₅-HLA-DRB1*0402/DRA, DG3₂₅₁₋₂₆₅-HLA-DRB1*0401/DRA, DG3₄₄₁₋₄₅₅-HLA-DRB1*0402/DRA, DG3₃₅₁₋₃₆₅-HLA-DRB3*0202/DRA, DG3₄₅₃₋₄₆₇-HLA-DRB3*0202/DRA, DG3₅₄₀₋₅₅₄-HLA-DRB3*0202/DRA, DG3₂₈₀₋₂₉₄-HLA-DRB4*0101/DRA, DG3₃₂₆₋₃₄₀-HLA-DRB4*0101/DRA, DG3₃₆₇₋₃₈₁-HLA-DRB4*0101/DRA, DG3₁₃₋₂₇-HLA-DRB5*0101/DRA, DG3₃₂₃₋₃₃₇-HLA-DRB5*0101/DRA, DG3₄₃₈₋₄₅₂-HLA-DRB5*0101/DRA, DG1₄₈₋₆₂-HLA-DRB3*0202/DRA, DG1₂₀₆₋₂₂₂-HLA-DRB3*0202/DRA, DG1₃₆₃₋₃₇₇-HLA-DRB3*0202/DRA, DG1₃₋₁₇-HLA-DRB4*0101/DRA, DG1₁₉₂₋₂₀₆-HLA-DRB4*0101/DRA, DG1₃₂₆₋₃₄₀-HLA-DRB4*0101/DRA, DG1₁₋₁₅-HLA-DRB5*0101/DRA, DG1₃₅₋₄₉-HLA-DRB5*0101/DRA, or DG1₃₂₅₋₃₃₉-HLA-DRB5*0101/DRA;
f) neuromyelitis optica spectrum disorder and the pMHC complex is selected from the group of: AQP4₁₂₉₋₁₄₃-HLA-DRB1*0101/DRA, AQP4₂₈₄₋₂₉₈-HLA-DRB1*0301/DRA, AQP4₆₃₋₇₆-HLA-DRB1*0301/DRA, AQP4₁₂₉₋₁₄₃-HLA-DRB1*0401/DRA, or AQP4₃₉₋₅₃-HLA-DRB1*1501/DRA;
g) allergic asthma and the pMHC complex is selected from the group of: DERP-1₁₆₋₃₀-HLA-DRB1*0101/DRA, DERP-1₁₆₋₃₀-HLA-DRB1*1501/DRA, DERP₁₁₇₁₋₁₈₅-HLA-DRB1*1501/DRA, DERP-1₁₁₀₋₁₂₄-HLA-DPB1*0401/DRA, DERP-2₂₆₋₄₀-HLA-DRB1*0101/DRA; DERP-2₂₆₋₄₀-HLA-DRB1*1501/DRA, or DERP-2₁₀₇₋₁₂₁-HLA-DRB1*0301/DRA;
h) inflammatory bowel disease and the pMHC complex is selected from the group of: bacteroides integrase antigen₁₈₃₋₁₉₇-HLA-DRB3*0101/DRA, bacteroides integrase antigen₁₄₆₋₁₆₀-HLA-DRB3*0101/DRA, bacteroides integrase antigen₁₇₅₋₁₈₉-HLA-DRB3*0101/DRA, bacteroides integrase antigen₁₋₁₅-HLA-DRB5*0101/DRA, bacteroides integrase antigen₁₈₃₋₁₉₇-HLA-DRB5*0101/DRA, bacteroides integrase antigen₁₈₃₋₁₉₇-HLA-DRB3*0101/DRA, bacteroides integrase antigen₃₀₋₄₄-HLA-DRB5*0101/DRA, bacteroides integrase antigen₇₀₋₈₄-HLA-DRB4*0101/DRA, bacteroides integrase antigen₃₃₇₋₃₅₁-HLA-DRB4*0101/DRA, bacteroides integrase antigen₁₇₁₋₁₈₅-HLA-DRB4*0101/DRA, bacteroides integrase antigen₄₋₁₈-HLA-DRB3*0202/DRA, bacteroides integrase antigen₁₇₁₋₁₈₅-HLA-DRB3*0202/DRA, bacteroides integrase antigen₂₅₆₋₂₇₀-HLA-DRB3*0202/DRA, Fla-2/Fla-X₃₆₆₋₃₈₀-HLA-DRB3*0101/DRA, Fla-2/Fla-X₁₆₄₋₁₇₈-HLA-DRB3*0101/DRA, Fla-2/Fla-X₂₆₁₋₂₇₅-HLA-DRB5*0101/DRA, Fla-2/Fla-X₁₋₁₈-HLA-DRB5*0101/DRA, Fla-2/Fla-X₅₁₋₆₅-HLA-DRB4*0101/DRA, Fla-2/Fla-X₂₆₉₋₂₈₃-HLA-DRB4*0101/DRA, Fla-2/Fla-X₄₋₁₈-HLA-DRB3*0202/DRA, Fla-2/Fla-X₂₆₁₋₂₇₅-HLA-DRB3*0202/DRA, Fla-2/Fla-X₂₇₁₋₂₈₅-HLA-DRB3*0202/DRA, YIDX₇₈₋₉₂-HLA-DRB3*0101/DRA, YIDX₇₈₋₉₂-HLA-DRB4*0101/DRA, YIDX₉₃₋₁₀₇-HLA-DRB3*0101/DRA, YIDX₉₈₋₁₁₂-HLA-DRB5*0101/DRA, YIDX₂₃₋₃₇-HLA-DRB5*0101/DRA, YIDX₇₈₋₉₂-HLA-DRB4*0101/DRA, YIDX₁₉₅₋₂₀₉-HLA-DRB4*0101/DRA, YIDX₂₂₋₃₆-HLA-DRB3*0202/DRA, YIDX₈₀₋₉₄-HLA-DRB3*0202/DRA, or YIDX₁₀₁₋₁₁₅-HLA-DRB3*0202/DRA;
i) COPD and/or emphysema and the pMHC complex is selected from the group of: elastin₈₉₋₁₀₃-HLA-DRB3*0101/DRA, elastin₆₉₈₋₇₁₂-HLA-DRB5*0101/DRA, elastin₈₋₂₂-HLA-DRB5*0101/DRA, elastin₉₄₋₁₀₈-HLA-DRB5*0101/DRA, elastin₁₃₋₂₇-HLA-DRB4*0101/DRA, elastin₆₉₅₋₇₀₉-HLA-DRB4*0101/DRA, elastin₅₆₃₋₅₇₇-HLA-DRB4*0101/DRA, elastin₅₅₈₋₅₇₂-HLA-DRB4*0101/DRA, elastin₆₉₈₋₇₁₂-HLA-DRB5*0101/DRA, elastin₅₆₆₋₅₈₀-HLA-DRB3*0202/DRA, or elastin₆₄₅₋₆₅₉-HLA-DRB3*0202/DRA;
j) psoriasis and the pMHC complex is selected from the group of: Cap18₆₄₋₇₈-HLA-DRB3*0101/DRA, Cap18₃₄₋₄₈-HLA-DRB3*0101/DRA, Cap18₄₇₋₆₁-HLA-DRB3*0101/DRA, Cap18₁₅₁₋₁₆₅-HLA-DRB4*0101/DRA, Cap18₁₄₉₋₁₆₃-HLA-DRB5*0101/DRA, Cap18₁₅₂₋₁₆₆-HLA-DRB5*0101/DRA, Cap18₁₃₁₋₁₄₅-HLA-DRB5*0101/DRA, Cap18₂₄₋₃₈-HLA-DRB3*0202/DRA, ADMTSL5₂₄₅₋₂₅₉-HLA-DRB3*0101/DRA, ADMTSL5₂₆₇₋₂₈₁-HLA-DRB3*0101/DRA, ADMTSL5₃₇₂₋₃₈₆-HLA-DRB3*0101/DRA, ADMTSL5₂₈₉₋₃₀₃-HLA-DRB4*0101/DRA, ADMTSL5₃₉₆₋₄₁₀-HLA-DRB4*0101/DRA, ADMTSL5₄₃₃₋₄₄₇-HLA-DRB4*0101/DRA, ADMTSL5₁₄₂₋₁₅₆-HLA-DRB5*0101/DRA, ADMTSL5₂₃₆₋₂₅₀-HLA-DRB5*0101/DRA, ADMTSL5₃₀₁₋₃₁₅-HLA-DRB5*0101/DRA, ADMTSL5₂₀₃₋₂₁₇-HLA-DRB3*0202/DRA, ADMTSL5₄₀₄₋₄₁₈-HLA-DRB3*0202/DRA, or ADMTSL5₄₃₃₋₄₄₇-HLA-DRB3*0202/DRA;
k) autoimmune hepatitis and the pMHC complex is selected from the group of: CYP2D6₁₉₃₋₂₀₇-HLA-DRB1*0301/DRA, CYP2D6₇₆₋₉₀-HLA-DRB1*0301/DRA, CYP2D6₂₉₃₋₃₀₇-HLA-DRB1*0301/DRA, CYP2D6₃₁₃₋₃₃₂-HLA-DRB1*0301/DRA, CYP2D6₃₉₃₋₄₁₂-HLA-DRB1*0301/DRA, CYP2D6₁₉₉₋₂₁₃-HLA-DRB1*0401/DRA, CYP2D6₄₅₀₋₄₆₄-HLA-DRB1*0401/DRA, CYP2D6₃₀₁₋₃₁₅-HLA-DRB1*0401/DRA, CYP2D6₄₅₂₋₄₆₆-HLA-DRB1*0701/DRA, CYP2D6₅₉₋₇₃-HLA-DRB1*0701/DRA, CYP2D6₁₃₀₋₁₄₄-HLA-DRB1*0701/DRA, CYP2D6₁₉₃₋₂₁₂-HLA-DRB1*0701/DRA, CYP2D6₃₀₅₋₃₂₄-HLA-DRB1*0701/DRA, CYP2D6₁₃₁₋₁₄₅-HLA-DRB3*0202/DRA, CYP2D6₂₁₆₋₂₃₀-HLA-DRB3*0202/DRA, CYP2D6₂₃₈₋₂₅₂-HLA-DRB3*0202/DRA, CYP2D6₁₉₉₋₂₁₃-HLA-DRB4*0101/DRA, CYP2D6₂₃₅₋₂₅₂-HLA-DRB4*0101/DRA, CYP2D6₂₉₃₋₃₀₇-HLA-DRB4*0101/DRA, CYP2D6₂₃₈₋₂₅₂-HLA-DRB5*0101/DRA, CYP2D6₃₈₁₋₃₉₅-HLA-DRB5*0101/DRA, CYP2D6₄₂₉₋₄₄₃-HLA-DRB5*0101/DRA, SLA₃₃₄₋₃₄₈-HLA-DRB1*0301/DRA, SLA₁₉₆₋₂₁₀-HLA-DRB1*0301/DRA, SLA₁₁₅₋₁₂₉-HLA-DRB1*0301/DRA, SLA₃₇₃₋₃₈₆-HLA-DRB1*0301/DRA, SLA₁₈₆₋₁₉₇-HLA-DRB1*0301/DRA, SLA₃₁₇₋₃₃₁-HLA-DRB1*0401/DRA, SLA₁₇₁₋₁₈₅-HLA-DRB1*0401/DRA, SLA₄₁₇₋₄₃₁-HLA-DRB1*0401/DRA, SLA₃₅₉₋₃₇₃-HLA-DRB1*0701/DRA, SLA₂₁₅₋₂₂₉-HLA-DRB1*0701/DRA, SLA₁₁₁₋₁₂₅-HLA-DRB1*0701/DRA, SLA₁₁₀₋₁₂₄-HLA-DRB3*0202/DRA, SLA₂₉₉₋₃₁₃-HLA-DRB3*0202/DRA, SLA₃₄₂₋₃₅₆-HLA-DRB3*0202/DRA, SLA₄₉₋₆₃-HLA-DRB4*0101/DRA, SLA₁₁₉₋₁₃₃-HLA-DRB4*0101/DRA, SLA₂₆₀₋₂₇₄-HLA-DRB4*0101/DRA, SLA₂₆₋₄₀-HLA-DRB5*0101/DRA, SLA₈₆₋₁₀₀-HLA-DRB5*0101/DRA, or SLA₃₃₁₋₃₄₅-HLA-DRB5*0101/DRA;
l) uveitis and the pMHC complex is selected from the group of: arrestin₁₉₉₋₂₁₃-HLA-DRB3*0101/DRA, arrestin₇₇₋₉₁-HLA-DRB3*0101/DRA, arrestin₂₅₀₋₂₆₄-HLA-DRB3*0101/DRA, arrestin₁₇₂₋₁₈₆-HLA-DRB4*0101/DRA, arrestin₃₅₄₋₃₆₈-HLA-DRB4*0101/DRA, arrestin₂₃₉₋₂₅₃-HLA-DRB4*0101/DRA, arrestin₁₀₂₋₁₁₆-HLA-DRB5*0101/DRA, arrestin₅₉₋₇₃-HLA-DRB5*0101, arrestin₂₈₀₋₂₉₄-HLA-DRB5*0101, arrestin₂₉₁₋₃₀₆-HLA-DRB1*0301/DRA, arrestin₁₉₅₋₂₀₉-HLA-DRB3*0202/DRA, arrestin₁₉₉₋₂₁₃-HLA-DRB3*0202/DRA, or arrestin₂₀₀₋₂₁₄-HLA-DRB3*0202/DRA;
m) Sjogren Syndrome and the pMHC complex is selected from the group of: RO60₁₂₇₋₁₄₁-HLA-DRB1*0301/DRA, RO60₅₂₃₋₅₃₇-HLA-DRB1*0301/DRA, RO60₂₄₃₋₂₅₇-HLA-DRB1*0301/DRA, RO60₄₈₄₋₄₉₈-HLA-DRB3*0101/DRA, RO60₃₄₇₋₃₆₁-HLA-DRB3*0101/DRA, RO60₃₆₉₋₃₈₃-HLA-DRB3*0101/DRA, RO60₄₂₆₋₄₄₀-HLA-DRB4*0101/DRA, RO60₂₆₇₋₂₈₁-HLA-DRB4*0101/DRA, RO60₁₇₈₋₁₉₂-HLA-DRB4*0101/DRA, RO60₃₅₈₋₃₇₂-HLA-DRB5*0101/DRA, RO60₃₅₈₋₃₇₂-HLA-DRB4*0101/DRA, RO60₂₂₁₋₂₃₅-HLA-DRB5*0101/DRA, RO60₂₂₁₋₂₃₅-HLA-DRB4*0101/DRA, RO60₃₁₈₋₃₃₂-HLA-DRB5*0101/DRA, RO60₃₁₈₋₃₃₂-HLA-DRB4*0101/DRA, RO60₄₀₇₋₄₂₁-HLA-DRB4*0101/DRA, RO60₄₀₇₋₄₂₁-HLA-DQA1*0501/HLA-DQB1*0201, RO60₄₅₉₋₄₇₃-HLA-DRB4*0101/DRA, RO60₄₅₉₋₄₇₃-HLA-DQA1*0501/HLA-DQB1*0201, RO60₃₁₈₋₃₃₂-HLA-DQA1*0501/HLA-DQB1*0201, RO60₅₁₋₆₅-HLA-DRB3*0202/DRA, RO60₃₁₂₋₃₂₆-HLA-DRB3*0202/DRA, RO60₃₄₇₋₃₆₁-HLA-DRB3*0202/DRA, LA₂₄₁₋₂₅₅-HLA-DRB1*0301/DRA, LA₁₀₁₋₁₁₅-HLA-DRB1*0301/DRA, LA₁₅₃₋₁₆₇-HLA-DRB1*0301/DRA, LA₁₇₈₋₁₉₂-HLA-DRB3*0101/DRA, LA₁₉₋₃₃-HLA-DRB3*0101/DRA, LA₃₇₋₅₁-HLA-DRB3*0101/DRA, LA₁₃₃₋₁₄₇-HLA-DRB4*0101/DRA, LA₅₀₋₆₄-HLA-DRB4*0101/DRA, LA₃₂₄₆-HLA-DRB4*0101/DRA, LA₁₅₃₋₁₆₇-HLA-DRB5*0101/DRA, LA₈₃₋₉₇-HLA-DRB5*0101/DRA, LA₁₃₆₋₁₅₀-HLA-DRB5*0101/DRA, LA₂₉₇₋₃₁₁-HLA-DQA1*0501/HLA-DQB1*0201, LA₅₉₋₇₃-HLA-DQA1*0501/HLA-DQB1*0201, LA₅₉₋₇₃-HLA-DRB4*0101/DRA, LA₁₅₁₋₁₆₅-HLA-DQA1*0501/HLA-DQB1*0201, LA₁₅₁₋₁₆₅-HLA-DRB4*0101/DRA, LA₂₉₇₋₃₁₁-HLA-DRB4*0101/DRA, LA₅₀₋₆₄-HLA-DRB3*0202/DRA, LA₈₆₋₁₀₀-HLA-DRB3*0202/DRA, or LA₁₅₄₋₁₆₈-HLA-DRB3*0202/DRA;
n) scleroderma and the pMHC complex is selected from the group of: TOP1₃₄₆₋₃₆₀-HLA-DRB3*0101/DRA, TOP1₄₂₀₋₄₃₄-HLA-DRB3*0101/DRA, TOP1₇₅₀₋₇₆₄-HLA-DRB3*0101/DRA, TOP1₄₁₉₋₄₃₃-HLA-DRB4*0101/DRA, TOP1₅₉₁₋₆₀₅-HLA-DRB4*0101/DRA, TOP1₆₉₅₋₇₀₉-HLA-DRB4*0101/DRA, TOP1₃₀₅₋₃₁₉-HLA-DRB5*0101/DRA, TOP1₃₄₆₋₃₆₀-HLA-DRB5*0101/DRA, TOP1₄₁₉₋₄₃₃-HLA-DRB5*0101/DRA, TOP1₄₂₀₋₄₃₄-HLA-DRB3*0202/DRA, TOP1₄₂₅₋₄₃₉-HLA-DRB3*0202/DRA, TOP1₆₁₄₋₆₂₈-HLA-DRB3*0202/DRA, CENP-C₂₉₇₋₃₁₁-HLA-DRB3*0101/DRA, CENP-C₈₅₇₋₈₇₁-HLA-DRB3*0101/DRA, CENP-C₈₈₇₋₉₀₁-HLA-DRB3*0101/DRA, CENP-C₂₁₂₋₂₂₆-HLA-DRB4*0101/DRA, CENP-C₆₄₃₋₆₅₇-HLA-DRB4*0101/DRA, CENP-C₈₃₂₋₈₄₆-HLA-DRB4*0101/DRA, CENP-C₁₆₇₋₁₈₁-HLA-DRB5*0101/DRA, CENP-C₂₄₆₋₂₆₀-HLA-DRB5*0101/DRA, CENP-C₈₄₆₋₈₆₀-HLA-DRB5*0101/DRA, CENP-C₁₄₉₋₁₆₃-HLA-DRB3*0202/DRA, CENP-C₈₃₃₋₈₄₇-HLA-DRB3*0202/DRA, or CENP-C₈₄₇₋₈₆₁-HLA-DRB3*0202/DRA;
o) anti-phospholipid syndrome and the pMHC complex is selected from the group of: APOH₂₃₅₋₂₄₉-HLA-DRB3*0101/DRA, APOH₃₀₆₋₃₂₀-HLA-DRB3*0101/DRA, APOH₂₃₇₋₂₅₁-HLA-DRB3*0101/DRA, APOH₂₉₅₋₃₀₉-HLA-DRB3*0101/DRA, APOH₂₈₋₄₂-HLA-DRB4*0101/DRA, APOH₁₇₃₋₁₈₇-HLA-DRB4*0101/DRA, APOH₂₆₄₋₂₇₈-HLA-DRB4*0101/DRA, APOH₂₉₅₋₃₀₉-HLA-DRB4*0101/DRA, APOH₄₉₋₆₃-HLA-DRB5*0101/DRA, APOH₂₆₉₋₂₈₃-HLA-DRB5*0101/DRA, APOH₂₉₅₋₃₀₉-HLA-DRB5*0101/DRA, APOH₃₂₁₋₃₅₅-HLA-DRB3*0202/DRA, APOH₃₂₂₋₃₃₆-HLA-DRB3*0202/DRA, or APOH₃₂₄₋₃₃₈-HLA-DRB3*0202/DRA;
p) ANCA-associated vasculitis and the pMHC complex is selected from the group of: MPO₅₀₆₋₅₂₀-HLA-DRB3*0101/DRA, MPO₃₀₂₋₃₁₆-HLA-DRB3*0101/DRA, MPO₇₋₂₁-HLA-DRB3*0101/DRA, MPO₆₈₉₋₇₀₃-HLA-DRB4*0101/DRA, MPO₂₄₈₋₂₆₂-HLA-DRB4*0101/DRA, MPO₄₄₄₋₄₅₈-HLA-DRB4*0101/DRA, MPO₅₁₃₋₅₂₇-HLA-DRB5*0101/DRA, MPO₉₇₋₁₁₁-HLA-DRB5*0101/DRA, MPO₆₁₆₋₆₃₀-HLA-DRB5*0101/DRA, MPO₄₆₂₋₄₇₆-HLA-DRB3*0202/DRA, MPO₆₁₇₋₆₃₁-HLA-DRB3*0202/DRA, MPO₇₁₄₋₇₂₈-HLA-DRB3*0202/DRA, PRTN3₄₄₋₅₈-HLA-DRB3*0101/DRA, PRTN3₂₃₄₋₂₄₈-HLA-DRB3*0101/DRA, PRTN3₅₉₋₇₃-HLA DRB3*0101/DRA, PRTN3₅₉₋₇₃-HLA-DRB5*0101/DRA, PRTN3₁₁₇₋₁₃₁-HLA-DRB4*0101/DRA, PRTN3₁₆₄₋₁₇₈-HLA-DRB4*0101/DRA, PRTN3₇₁₋₅₅-HLA-DRB4*0101/DRA, PRTN3₂₄₁₋₂₅₅-HLA-DRB5*0101/DRA, PRTN3₁₈₃₋₁₉₇-HLA-DRB5*0101/DRA, PRTN3₆₂₋₇₆-HLA-DRB3*0202/DRA, PRTN3₁₁₈₋₁₃₂-HLA-DRB3*0202/DRA, or PRTN3₂₃₉₋₂₅₃-HLA-DRB3*0202/DRA; or
q) Stiff Man Syndrome and the pMHC complex is selected from the group of: GAD₂₁₂₋₂₂₆-HLA-DRB1*0801/DRA, GAD₅₅₅₋₅₆₉-HLA-DRB1*0801/DRA, or GAD₂₉₇₋₃₁₁-HLA-DRB1*0301/DRA.

70. The complex of any of claims 37-46 and 49-69, wherein the pMHC complex is for the treatment of:
a) type I diabetes and the pMHC complex is selected from the group of: PPI_{76-90(K88S)}-HLA-DRB1*0401/DRA, IGRP₁₃₋₂₅-HLA-DRB1*0301/DRA, GAD₅₅₅₋₅₆₇ HLA-DRB1*0401/DRA, GAD_{555-567(557I)}-HLA-DRB1*0401/DRA, IGRP₂₃₋₃₅-HLA-DRB1*0401/DRA, or PPI₇₆₉₀-HLA-DRB1*0401/DRA;
b) multiple sclerosis and the pMHC complex is selected from the group of: MBP₈₆₋₉₈-HLA-DRB1*1501/DRA, MBP₈₉₋₁₀₁-HLA-DRB5*0101/DRA, MOG₃₈₋₅₂-HLA-DRB4*0101/DRA, MOG_{97-109(E107S)}-HLA-DRB1*0401/DRA, MOG₂₀₃₋₂₁₇-HLA-DRB3*0101/DRA, PLP₅₄₋₆₈-HLA-DRB3*0101/DRA, PLP₉₄₋₁₀₈-HLA-DRB1*0301/DRA, PLP₂₅₀₋₂₆₄-HLA-DRB4*0101/DRA, MPB₁₃₋₃₂-HLA-DRB5*0101/DRA, MPB₈₃₋₉₉-HLA-DRB5*0101/DRA, MPB₁₁₁₋₁₂₉-HLA-DRB5*0101/DRA, MPB₁₄₆₋₁₇₀-HLA-DRB5*0101/DRA, MOG₂₂₃₋₂₃₇-HLA-DRB3*0202/DRA, MOG₆₋₂₀-HLA-DRB5*0101/DRA, PLP₈₈₋₁₀₂-HLA-DRB3*0202/DRA, or PLP₁₃₉₋₁₅₄-HLA-DRB5*0101/DRA;
c) Celiac Disease and the pMHC complex is selected from the group of: aGlia₅₇₋₆₈-HLA-DQA1*0501/HLA-DQB1*0201, aGlia₆₂₋₇₂-HLA-DQA1*0501/HLA-DQB1*0201, or aGlia₂₁₇₋₂₂₉-HLA-DQA1*0501/HLA-DQB1*0302;
d) primary biliary cirrhosis and the pMHC complex is selected from the group of: PDC-E2₁₂₂₋₁₃₅-HLA-DRB4*0101/DRA, PDC-E2₂₄₉₋₂₆₂-HLA-DRB4*0101/DRA, PDC-E2₂₄₉₋₂₆₃-HLA-DRB1*0801/DRA, PDC-E2₆₂₉₋₆₄₃-HLA-DRB1*0801/DRA, PDC-E2₇₂₋₈₆-HLA-DRB3*0202/DRA, PDC-E2₃₅₃₋₃₆₇-HLA-DRB3*0202/DRA, PDC-E2₄₂₂₋₄₃₆-HLA-DRB3*0202/DRA, PDC-E2₆₂₉₋₆₄₃-HLA-DRB4*0101/DRA, PDC-E2₈₀₋₉₄-HLA-DRB5*0101/DRA, PDC-E2₃₅₃₋₃₆₇-HLA-DRB5*0101/DRA, or PDC-E2₅₃₅₋₅₄₉-HLA-DRB5*0101/DRA;
e) pemphigus folliaceus and/or pemphigus vulgaris and the pMHC complex is selected from the group of: DG1₂₁₆₋₂₂₉-HLA-DRB1*0101/DRA, DG3₉₇₋₁₁₁-HLA-DRB1*0402/DRA, DG3₂₅₁₋₂₆₅-HLA-DRB1*0401/DRA, DG3₄₄₁₋₄₅₅-HLA-DRB1*0402/DRA, DG3₃₅₁₋₃₆₅-HLA-DRB3*0202/DRA, DG3₄₅₃₋₄₆₇-HLA-DRB3*0202/DRA, DG3₅₄₀₋₅₅₄-HLA-DRB3*0202/DRA, DG3₂₈₀₋₂₉₄-HLA-DRB4*0101/DRA, DG3₃₂₆₋₃₄₀-HLA-DRB4*0101/DRA, DG3₃₆₇₋₃₈₁-HLA-DRB4*0101/DRA, DG3₁₃₋₂₇-HLA-DRB5*0101/DRA, DG3₃₂₃₋₃₃₇-HLA-DRB5*0101/DRA, DG3₄₃₈₋₄₅₂-HLA-DRB5*0101/DRA, DG1₄₈₋₆₂-HLA-DRB3*0202/DRA, DG1₂₀₆₋₂₂₂-HLA-DRB3*0202/DRA, DG1₃₆₃₋₃₇₇-HLA-DRB3*0202/DRA, DG1₃₋₁₇-HLA-DRB4*0101/DRA, DG1₁₉₂₋₂₀₆-HLA-DRB4*0101/DRA, DG1₃₂₆₋₃₄₀-HLA-DRB4*0101/DRA, DG1₁₋₁₅-HLA-DRB5*0101/DRA, DG1₃₅₋₄₉-HLA-DRB5*0101/DRA, or DG1₃₂₅₋₃₃₉-HLA-DRB5*0101/DRA;
f) neuromyelitis optica spectrum disorder and the pMHC complex is selected from the group of: AQP4₂₈₄₋₂₉₈-HLA-DRB1*0301/DRA, AQP4₆₃₋₇₆-HLA-DRB1*0301/DRA, AQP4₁₂₉₋₁₄₃-HLA-DRB1*0401/DRA, or AQP4₃₉₋₅₃-HLA-DRB1*1501/DRA;
g) allergic asthma and the pMHC complex is selected from the group of: DERP-1₁₆₋₃₀-HLA-DRB1*0101/DRA, DERP-1₁₆₋₃₀-HLA-DRB1*1501/DRA, DERP1₁₇₁₋₁₈₅-HLA-DRB1*1501/DRA, DERP-1₁₁₀₋₁₂₄-HLA-DPB1*0401/DRA, DERP-2₂₆₋₄₀-HLA-DRB1*0101/DRA; DERP-2₂₆₋₄₀-HLA-DRB1*1501/DRA, or DERP-2₁₀₇₋₁₂₁-HLA-DRB1*0301/DRA;
h) inflammatory bowel disease and the pMHC complex is selected from the group of: bacteroides integrase antigen₁₋₁₅-HLA-DRB5*0101/DRA, bacteroides integrase antigen₁₈₃₋₁₉₇-HLA-DRB3*0101/DRA, bacteroides integrase antigen₇₀₋₈₄-HLA-DRB4*0101/DRA, bacteroides integrase antigen₄₋₁₈-HLA-DRB3*0202/DRA, bacteroides integrase antigen₁₇₁₋₁₈₅-HLA-DRB3*0202/DRA, bacteroides integrase antigen₂₅₆₋₂₇₀-HLA-DRB3*0202/DRA, Fla-2/Fla-X₃₆₆₋₃₈₀-HLA-DRB3*0101/DRA, Fla-2/Fla-X₂₆₁₋₂₇₅-HLA-DRB5*0101/DRA, Fla-2/Fla-X₅₁₋₆₅-HLA-DRB4*0101/DRA, Fla-2/Fla-X₄₋₁₈-HLA-DRB3*0202/DRA, Fla-2/Fla-X₂₆₁₋₂₇₅-HLA-DRB3*0202/DRA, Fla-2/Fla-X₂₇₁₋₂₈₅-HLA-DRB3*0202/DRA, YIDX₇₈₋₉₂-HLA-DRB3*0101/DRA, YIDX₇₈₋₉₂-HLA-DRB4*0101/DRA, YIDX₉₈₋₁₁₂-HLA-DRB5*0101/DRA, YIDX₂₂₋₃₆-HLA-DRB3*0202/DRA, YIDX₈₀₋₉₄-HLA-DRB3*0202/DRA, or YIDX₁₀₋₁₁₅-HLA-DRB3*0202/DRA;
i) emphysema and the pMHC complex is selected from the group of: elastin₈₉₋₁₀₃-HLA-DRB3*0101/DRA, elastin₆₉₈₋₇₁₂-HLA-DRB5*0101/DRA, elastin₅₅₈₋₅₇₂-HLA-DRB4*0101/DRA, elastin₅₆₆₋₅₈₀-HLA-DRB3*0202/DRA, or elastin₅₄₅₋₆₅₉-HLA-DRB3*0202/DRA;
j) psoriasis and the pMHC complex is selected from the group of: Cap18₆₄₋₇₈-HLA-DRB3*0101/DRA, Cap18₃₄₋₄₈-HLA-DRB3*0101/DRA, Cap18₄₇₋₆₁-HLA-DRB3*0101/DRA, Cap18₁₅₁₋₁₆₅-HLA-DRB4*0101/DRA, Cap18₁₄₉₋₁₆₃-HLA-DRB5*0101/DRA, Cap18₁₅₂₋₁₆₆-HLA-DRB5*0101/DRA, Cap18₁₃₁₋₁₄₅-HLA-DRB5*0101/DRA, Cap₁₈₂₄₋₃₈-HLA-DRB3*0202/DRA, ADMTSL5₂₄₅₋₂₅₉-HLA-DRB3*0101/DRA, ADMTSL5₂₆₇₋₂₈₁-HLA-DRB3*0101/DRA, ADMTSL5₃₇₂₋₃₈₆-HLA-DRB3*0101/DRA, ADMTSL5₂₈₉₋₃₀₃-HLA-DRB4*0101/DRA, ADMTSL5₃₉₆₋₄₁₀-HLA-DRB4*0101/DRA, ADMTSL5₄₃₃₋₄₄₇-HLA-DRB4*0101/DRA, ADMTSL5₁₄₂₋₁₅₆-HLA-DRB5*0101/DRA, ADMTSL5₂₃₆₋₂₅₀-HLA-DRB5*0101/DRA, ADMTSL5₃₀₁₋₃₁₅-HLA-DRB5*0101/DRA, ADMTSL5₂₀₃₋₂₁₇-HLA-DRB3*0202/DRA, ADMTSL5₄₀₄₋₄₁₈-HLA-DRB3*0202/DRA, or ADMTSL5₄₃₃₋₄₄₇-HLA-DRB3*0202/DRA;
k) autoimmune hepatitis and the pMHC complex is selected from the group of: CYP2D6₁₉₃₋₂₀₇-HLA-DRB1*0301/DRA, CYP2D6₇₆₋₉₀-HLA-DRB1*0301/DRA, CYP2D6₂₉₃₋₃₀₇-HLA-DRB1*0301/DRA, CYP2D6₃₁₃₋₃₃₂-HLA-DRB1*0301/DRA, CYP2D6₃₉₃₋₄₁₂-HLA-DRB1*0301/DRA, CYP2D6₁₉₉₋₂₁₃-HLA-DRB1*0401/DRA, CYP2D6₄₅₀₋₄₆₄-HLA-DRB1*0401/DRA, CYP2D6₃₀₁₋₃₁₅-HLA-DRB1*0401/DRA, CYP2D6₄₅₂₋₄₆₆-HLA-DRB1*0701/DRA, CYP2D6₅₉₋₇₃-HLA-DRB1*0701/DRA, CYP2D6₁₃₀₋₁₄₄-HLA-DRB1*0701/DRA, CYP2D6₁₉₃₋₂₁₂-HLA-DRB1*0701/DRA, CYP2D6₃₀₅₋₃₂₄-HLA-DRB1*0701/DRA, CYP2D6₁₃₁₋₁₄₅-HLA-DRB3*0202/DRA, CYP2D6₂₁₆₋₂₃₀-HLA-DRB3*0202/DRA, CYP2D6₂₃₈₋₂₅₂-HLA-DRB3*0202/DRA, CYP2D6₁₉₉₋₂₁₃-HLA-DRB4*0101/DRA, CYP2D6₂₃₅₋₂₅₂-HLA-DRB4*0101/DRA, CYP2D6₂₉₃₋₃₀₇-HLA-DRB4*0101/DRA, CYP2D6₂₃₈₋₂₅₂-HLA-DRB5*0101/DRA, CYP2D6₃₈₁₋₃₉₅-HLA-DRB5*0101/DRA, CYP2D6₄₂₉₋₄₄₃-HLA-DRB5*0101/DRA, SLA₃₃₄₋₃₄₈-HLA-DRB1*0301/DRA, SLA₁₉₆₋₂₁₀-HLA-DRB1*0301/DRA, SLA₁₁₅₋₁₂₉-HLA-DRB1*0301/DRA, SLA₃₇₃₋₃₈₆-HLA-DRB1*0301/DRA, SLA₁₈₆₋₁₉₇-HLA-DRB1*0301/DRA, SLA₃₁₇₋₃₃₁-HLA-DRB1*0401/DRA, SLA₁₇₁₋₁₈₅-HLA-DRB1*0401/DRA, SLA₄₁₇₋₄₃₁-HLA-DRB1*0401/DRA, SLA₃₅₉₋₃₇₃-HLA-DRB1*0701/DRA, SLA₂₁₅₋₂₂₉-HLA-DRB1*0701/DRA, SLA₁₁₁₋₁₂₅-HLA-DRB1*0701/DRA, SLA₁₁₀₋₁₂₄-HLA-DRB3*0202/DRA, SLA₂₉₉₋₃₁₃-HLA-DRB3*0202/DRA, SLA₃₄₂₋₃₅₆-HLA-DRB3*0202/DRA, SLA₄₉₋₆₃-HLA-DRB4*0101/DRA, SLA₁₁₉₋₁₃₃-HLA-DRB4*0101/DRA, SLA₂₆₀₋₂₇₄-HLA-DRB4*0101/DRA, SLA₂₆₋₄₀-HLA-DRB5*0101/DRA, SLA₈₆₋₁₀₀-HLA-DRB5*0101/DRA, or SLA₃₃₁₋₃₄₅-HLA-DRB5*0101/DRA;
l) uveitis and the pMHC complex is selected from the group of: arrestin₁₉₉₋₂₁₃-HLA-DRB3*0101/DRA, arrestin₇₇₋₉₁-HLA-DRB3*0101/DRA, arrestin₂₅₀₋₂₆₄-HLA-DRB3*0101/DRA, arrestin₁₇₂₋₁₈₆-HLA-DRB4*0101/DRA, arrestin₃₅₄₋₃₆₈-HLA-DRB4*0101/DRA, arrestin₂₃₉₋₂₅₃-HLA-DRB4*0101/DRA, arrestin₁₀₂₋₁₁₆-HLA-DRB5*0101/DRA, arrestin₅₉₋₇₃-HLA-DRB5*0101, arrestin₂₈₀₋₂₉₄-HLA-DRB5*0101, arrestin₂₉₁₋₃₀₆-HLA-DRB1*0301/DRA, arrestin₁₉₅₋₂₀₉-HLA-DRB3*0202/DRA, arrestin₁₉₉₋₂₁₃-HLA-DRB3*0202/DRA, or arrestin₂₀₀₋₂₁₄-HLA-DRB3*0202/DRA;
m) Sjogren Syndrome and the pMHC complex is selected from the group of: RO60₁₂₇₋₁₄₁-HLA-DRB1*0301/DRA, RO60₅₂₃₋₅₃₇-HLA-DRB1*0301/DRA, RO60₂₄₃₋₂₅₇-HLA-DRB1*0301/DRA, RO60₄₈₄₋₄₉₈-HLA-DRB3*0101/DRA, RO60₃₄₇₋₃₆₁-HLA-DRB3*0101/DRA, RO60₃₆₉₋₃₈₃-HLA-DRB3*0101/DRA, RO60₄₂₆₋₄₄₀-HLA-DRB4*0101/DRA, RO60₂₆₇₋₂₈₁-HLA-DRB4*0101/DRA, RO60₁₇₈₋₁₉₂-HLA-DRB4*0101/DRA, RO60₃₅₈₋₃₇₂-HLA-DRB5*0101/DRA, RO60₂₂₁₋₂₃₅-HLA-DRB5*0101/DRA, RO60₃₁₈₋₃₃₂-HLA-DRB5*0101/DRA, RO60₅₁₋₆₅-HLA-DRB3*0202/DRA, RO60₃₁₂₋₃₂₆-HLA-DRB3*0202/DRA, RO60₃₄₇₋₃₆₁-HLA-DRB3*0202/DRA, LA₂₄₁₋₂₅₅-HLA-DRB1*0301/DRA, LA₁₀₁₋₁₁₅-HLA-DRB1*0301/DRA, LA₁₅₃₋₁₆₇-HLA-DRB1*0301/DRA, LA₁₇₈₋₁₉₂-HLA-DRB3*0101/DRA, LA₁₉₋₃₃-HLA-DRB3*0101/DRA, LA₃₇₋₅₁-HLA-DRB3*0101/DRA, LA₁₃₃₋₁₄₇-HLA-DRB4*0101/DRA, LA₅₀₋₆₄-HLA-DRB4*0101/DRA, LA₃₂₋₄₆-HLA-DRB4*0101/DRA, LA₁₅₃₋₁₆₇-HLA-DRB5*0101/DRA, LA₈₃₋₉₇-HLA-DRB5*0101/DRA, LA₁₃₆₋₁₅₀-HLA-DRB5*0101/DRA, LA₅₀₋₆₄-HLA-DRB3*0202/DRA, LA₈₆₋₁₀₀-HLA-DRB3*0202/DRA, or LA₁₅₄₋₁₆₈-HLA-DRB3*0202/DRA;
n) scleroderma and the pMHC complex is selected from the group of: TOP1₃₄₆₋₃₆₀-HLA-DRB3*0101/DRA, TOP1₄₂₀₋₄₃₄-HLA-DRB3*0101/DRA, TOP1₇₅₀₋₇₆₄-HLA-DRB3*0101/DRA, TOP1₄₁₉₋₄₃₃-HLA-DRB4*0101/DRA, TOP1₅₉₁₋₆₀₅-HLA-DRB4*0101/DRA, TOP1₆₉₅₋₇₀₉-HLA-DRB4*0101/DRA, TOP1₃₀₅₋₃₁₉-HLA-DRB5*0101/DRA, TOP1₃₄₆₋₃₆₀-HLA-DRB5*0101/DRA, TOP1₄₁₉₋₄₃₃-HLA-DRB5*0101/DRA, TOP1₄₂₀₋₄₃₄-HLA-DRB3*0202/DRA, TOP1₄₂₅₋₄₃₉-HLA-DRB3*0202/DRA, TOP1₆₁₄₋₆₂₈-HLA-DRB3*0202/DRA, CENP-C₂₉₇₋₃₁₁-HLA-DRB3*0101/DRA, CENP-C₈₅₇₋₈₇₁-HLA-DRB3*0101, CENP-C₈₈₇₋₉₀₁-HLA-DRB3*0101, CENP-C₂₁₂₋₂₂₆-HLA-DRB4*0101/DRA, CENP-C₆₄₃₋₆₅₇-HLA-DRB4*0101/DRA, CENP-C₈₃₂₋₈₄₆-HLA-DRB4*0101/DRA, CENP-C₁₆₇₋₁₈₁-HLA-DRB5*0101/DRA, CENP-C₂₄₆₋₂₆₀-HLA-DRB5*0101/DRA, CENP-C₈₄₆₋₈₆₀-HLA-DRB5*0101/DRA, CENP-C₁₄₉₋₁₆₃-HLA-DRB3*0202/DRA, CENP-C₈₃₃₋₈₄₇-HLA-DRB3*0202/DRA, or CENP-C₈₄₇₋₈₆₁-HLA-DRB3*0202/DRA;
o) anti-phospholipid syndrome and the pMHC complex is selected from the group of: APOH₂₃₅₋₂₄₉-HLA-DRB3*0101/DRA, APOH₃₀₆₋₃₂₀-HLA-DRB3*0101/DRA, APOH₂₃₇₋₂₅₁-HLA-DRB3*0101/DRA, APOH₂₉₅₋₃₀₉-HLA-DRB3*0101/DRA, APOH₂₈₋₄₂-HLA-DRB4*0101/DRA, APOH₁₇₃₋₁₈₇-HLA-DRB4*0101/DRA, APOH₂₆₄₋₂₇₈-HLA-DRB4*0101/DRA, APOH₂₉₅₋₃₀₉-HLA-DRB4*0101/DRA, APOH₄₉₋₆₃-HLA-DRB5*0101/DRA, APOH₂₆₉₋₂₈₃-HLA-DRB5*0101/DRA, APOH₂₉₅₋₃₀₉-HLA-DRB5*0101/DRA, APOH₃₂₁₋₃₅₅-HLA-DRB3*0202/DRA, APOH₃₂₂₋₃₃₆-HLA-DRB3*0202/DRA, or APOH₃₂₄₋₃₃₈-HLA-DRB3*0202/DRA;
p) ANCA-associated vasculitis and the pMHC complex is selected from the group of: MPO₅₀₆₋₅₂₀-HLA-DRB3*0101/DRA, MPO₃₀₂₋₃₁₆-HLA-DRB3*0101/DRA, MPO₇₋₂₁-HLA-DRB3*0101/DRA, MPO₆₈₉₋₇₀₃-HLA-DRB4*0101/DRA, MPO₂₄₈₋₂₆₂-HLA-DRB4*0101/DRA, MPO₄₄₄₋₄₅₈-HLA-DRB4*0101/DRA, MPO₅₁₃₋₅₂₇-HLA-DRB5*0101/DRA, MPO₉₇₋₁₁₁-HLA-DRB5*0101/DRA, MPO₆₁₆₋₆₃₀-HLA-DRB5*0101/DRA, MPO₄₆₂₋₄₇₆-HLA-DRB3*0202/DRA, MPO₆₁₇₋₆₃₁-HLA-DRB3*0202/DRA, MPO₇₁₄₋₇₂₈-HLA-DRB3*0202/DRA, PRTN3₄₄₋₅₈-HLA-DRB3*0101/DRA, PRTN3₂₃₄₋₂₄₈-HLA-DRB3*0101/DRA, PRTN3₅₉₋₇₃-HLA DRB3*0101/DRA, PRTN3₅₉₋₇₃-HLA-DRB5*0101/DRA, PRTN3₁₁₇₋₁₃₁-HLA-DRB4*0101/DRA, PRTN3₁₆₄₋₁₇₈-HLA-DRB4*0101/DRA, PRTN3₇₁₋₈₅-HLA-DRB4*0101/DRA, PRTN3₂₄₁₋₂₅₅-HLA-DRB5*0101/DRA, PRTN3₁₈₃₋₁₉₇-HLA-DRB5*0101/DRA, PRTN3₆₂₋₇₆-HLA-DRB3*0202/DRA, PRTN3₁₁₈₋₁₃₂-HLA-DRB3*0202/DRA, or PRTN3₂₃₉₋₂₅₃-HLA-DRB3*0202/DRA; or
q) Stiff Man Syndrome and the pMHC complex is selected from the group of: GAD₂₁₂₋₂₂₆-HLA-DRB1*0801/DRA, GAD₅₅₅₋₅₆₉-HLA-DRB1*0801/DRA, or GAD₂₉₇₋₃₁₁-HLA-DRB1*0301/DRA.

71. A composition comprising a plurality of complexes of any of claims 37-70 and a carrier.

72. The composition of claim 71, wherein one or more of:
each nanoparticle dendrimer core or polymeric micelle core are the same or different from each other; and/or the diameters of each nanoparticle dendrimer core or polymeric micelle core are the same or different from each other; and/or the valency of the pMHC complexes on each nanoparticle dendrimer core or polymeric micelle core are the same or different from each other; and/or the density of the pMHC complexes on each nanoparticle dendrimer core or polymeric micelle core are the same or different from each other; and/or the valency of the co-stimulatory molecules on each nanoparticle dendrimer core or polymeric micelle core are the same or different from each other; and/or the valency of the cytokines on each nanoparticle dendrimer core or polymeric micelle core are the same or different from each other.

73. The composition of claim 71 or 72, further comprising one or more of a nanoparticle dendrimer core or a polymeric micelle core coupled to one or more cytokines and/or co-stimulatory molecules,
wherein the nanoparticle core has a diameter selected from the group of: from about 1 nm to about 100 nm; from about 1 nm to about 50 nm; from about 1 nm to about 25 nm; from about 5 nm to about 100 nm; from about 5 nm to about 50 nm; or from about 5 nm to about 25 nm; and
wherein the nanoparticle dendrimer core or a polymeric micelle core optionally comprises an outer layer on the surface of the core.

74. A composition comprising a carrier and one or more of a complex of any of claims 37-70 and/or the composition of claims 71-73.

75. The composition of claim 74, wherein the carrier is a pharmaceutically acceptable carrier.

76. A method for differentiating a population of T cells to effector T cells, comprising contacting the T cells with an effective amount of the complex of any of claims 37-70 or the composition of claims 71-75, wherein optionally the pMHC density of the nanoparticle is from about 0.4 pMHC/100 nm² to about 12 pMHC/100 nm².

77. The method of claim 76, wherein the contacting is in vitro or in vivo.

78. A method for differentiating a population of T cells to effector T cells in a subject with cancer, comprising administering an effective amount of the complex of any of claims 37-70 or the composition of claims 71-75, with the proviso that that the disease-relevant antigen of the pMHC complex is a cancer-relevant antigen or a tumor-relevant antigen.

79. A method for differentiating an activated T cell or a memory T cell into a IL-10 producing T_{R}1 cell optionally expressing a marker CD49b and/or Lag3 and/or differentiating a B cell into a regulatory B cell comprising contacting the activated T cell or the memory T cell with an effective amount of the complex of any of claims 37-70 or the composition of claims 71-75, thereby differentiating the activated T cells or the memory T cells, wherein the pMHC density of the complex or the nanoparticle cores of the composition is from about 0.4 pMHC/100 nm² to about 12 pMHC/100 nm².

80. The method of claim 79, wherein the contacting is in vitro or in vivo.

81. A method for differentiating an activated T cell or a memory T cell into a IL-10 producing T_{R}1 cell optionally expressing a marker CD49b and/or LAG3 and/or differentiating a B cell into a regulatory B cell in a subject with an autoimmune disease, comprising administering to the subject an effective amount of the complex of any of claims 37-70 or the composition of claims 71-75, thereby differentiating the activated T cells or the memory T cells, wherein the pMHC density of the complex or the nanoparticle cores of the composition is from about 0.4 pMHC/100 nm² to about 12 pMHC/100 nm², with the proviso that that the disease-relevant antigen is not a cancer-relevant antigen or a tumor-relevant antigen.

82. A complex comprising a nanoparticle core coupled to a plurality of antigen-MHC complexes (pMHC) for use in expanding and/or developing populations of T effector cells in a subject, wherein the core of the nanoparticle has a diameter selected from the group of: from about 1 nm to about 100 nm; from about 1 nm to about 50 nm; from about 1 nm to about 25 nm; from about 5 nm to about 100 nm; from about 5 nm to about 50 nm; or from about 5 nm to about 25 nm, and optionally comprises an outer layer on the core; and wherein the valency of the pMHC complexes per nanoparticle core is from about 10:1 to about 6000:1; wherein the complex has a pMHC density from about 0.05 pMHC/100 nm² to about 25 pMHC/100 nm² of the surface area of the nanoparticle core; and optionally wherein the core further comprises a plurality of co-stimulatory molecules and/or a plurality of cytokines, and wherein the valency of the co-stimulatory and/or cytokine molecules per nanoparticle core is from about 1 to about 6000.

83. The complex of claim 82, wherein the plurality of co-stimulatory molecules per nanoparticle core are the same or different from each other and/or the plurality of cytokines per nanoparticle core are the same or different from each other.

84. The complex of claim 82 or 83, wherein the MHC protein of the pMHC complexes are selected from classical MHC class I protein, non-classical MHC class I protein, classical MHC class II protein, non-classical MHC class II protein, MHC dimers (Fc fusions), MHC tetramers and a polymeric form of a MHC protein, wherein the MHC protein optionally comprises a knob-in-hole based MHC-alpha-Fc/MHC-beta-Fc heterodimer or multimer.

85. The complex of any of claims 82-84, wherein the antigens of the plurality of pMHC complexes per nanoparticle core are the same or different from each other.

86. The complex of any of claims 82-85, wherein the disease-relevant antigen is a tumor or cancer-relevant antigen.

87. The complex of claim 86, wherein the tumor or cancer is a carcinoma, sarcoma, myeloma, leukemia, lymphoma, melanoma, or mixed type or a metastases thereof.

88. The complex of any of claims 82-87, wherein the nanoparticle core is selected from a solid core, a metal core, a dendrimer core, a polymeric micelle nanoparticle core, a nanorod, a fullerene, an nanoshell, a coreshell, a protein-based nanostructure or a lipid-based nanostructure.

89. The complex of any of claims 82-88, wherein the nanoparticle core is non-liposomal.

90. The complex of any of claims 80-89, wherein the pMHC complex is coupled to the nanoparticle core by one or more of covalently, non-covalently, or cross-linked and optionally coupled through a linker to the core or the outer layer.

91. The complex of claim 90, wherein the linker is less than 5 kD in size, that is optionally polyethylene glycol.

92. The complex of claim 90 or 91, wherein the linkers per nanoparticle core are identical or different from each other.

93. The complex of any of claims 82-92, wherein the nanoparticle core and/or the outer layer is bioabsorbable and/or biodegradable.

94. The complex of any of claims 82-93, wherein the outer layer comprises polyethylene glycol.

95. The complex of any of claims 82-94, wherein the valency of the pMHC complex is from about 10:1 to about 1000:1 per nanoparticle core.

96. The complex of any of claims 84-95, wherein the density of the pMHC complexes comprises from about 0.4 pMHC/100 nm² of surface area of the nanoparticle to about 25 pMHC/100 nm² of surface area of the nanoparticle

97. The complex of any of claims 82-96, wherein the MHC protein of the pMHC complex comprises a classical or a non-classical MHC class I protein and/or a classical or non-classical MHC class II protein wherein each optionally is the same or different from each other.

98. The complex of any of claims 82-97, wherein the MHC protein of the pMHC complex comprises HLA DR, HLA DQ, HLA DP, HLA-A, HLA-B, HLA-C, HLA-E, HLA-F, HLA-G, CD1d, or a fragment or a biological equivalent of each thereof.

99. The complex of any of claims 82-98, wherein the pMHC density is from about 0.4 pMHC/100 nm² to about 6 pMHC/100 nm² and wherein the nanoparticle core has a diameter of from about 15 nm to about 25 nm.

100. The complex of any of claims 82-99, wherein on a single nanoparticle core: the pMHC complexes are the same or different from each other; and/or the MHC protein of the pMHC complexes are the same or different from each other; and/or the costimulatory molecules are the same or different from each other; and/or the cytokines are the same or different from each other.

101. The complex of any of claims 82-85 and 88-100, wherein the disease-relevant antigen is:
a) a diabetes-relevant antigen and is derived from an antigen selected from one or more of the group: preproinsulin (PPI), islet-specific glucose-6-phosphatase (IGRP), glutamate decarboxylase (GAD), islet cell autoantigen-2 (ICA2), insulin, proinsulin, or a fragment or an equivalent of each thereof;
b) a multiple sclerosis-relevant antigen and is derived from an antigen selected from one or more of the group: myelin basic protein, myelin associated glycoprotein, myelin oligodendrocyte protein, proteolipid protein, oligodendrocyte myelin oligoprotein, myelin associated oligodendrocyte basic protein, oligodendrocyte specific protein, heat shock proteins, oligodendrocyte specific proteins, NOGO A, glycoprotein Po, peripheral myelin protein 22, 2′3′-cyclic nucleotide 3′-phosphodiesterase, or a fragment or an equivalent of each thereof;
c) a Celiac Disease-relevant antigen and is derived from gliadin or a fragment or an equivalent thereof;
d) a primary biliary cirrhosis-relevant antigen and is derived from PDC-E2 or a fragment or an equivalent thereof;
e) a pemphigus folliaceus-relevant antigen and/or pemphigus vulgaris-relevant antigen and is derived from an antigen selected from one or more of the group: DG1, DG3, or a fragment or an equivalent of each thereof;
f) a neuromyelitis optica spectrum disorder-relevant antigen and is derived from AQP4 or a fragment or an equivalent thereof;
g) an arthritis-relevant antigen and is derived from an antigen selected from one or more of the group: heat shock proteins, immunoglobulin binding protein, heterogeneous nuclear RNPs, annexin V, calpastatin, type II collagen, glucose-6-phosphate isomerase, elongation factor human cartilage gp39, mannose binding lectin, citrullinated vimentin, type II collagen, fibrinogen, alpha enolase, anti-carbamylated protein (anti-CarP), peptidyl arginine deiminase type 4 (PAD4), BRAF, fibrinogen gamma chain, inter-alpha-trypsin inhibitor heavy chain H1, alpha-1-antitrypsin, plasma protease C1 inhibitor, gelsolin, alpha 1-B glycoprotein, ceruloplasmin, inter-alpha-trypsin inhibitor heavy chain H4, complement factor H, alpha 2 macroglobulin, serum amyloid, C-reactive protein, serum albumin, fibrogen beta chain, serotransferin, alpha 2 HS glycoprotein, vimentin, Complement C3, or a fragment or an equivalent of each thereof;
h) an allergic asthma-relevant antigen and is derived from an antigen selected from one or more of the group: DERP1, DERP2, or a fragment or an equivalent of each thereof;
i) an inflammatory bowel disease-relevant antigen and is derived from an antigen selected from one or more of the group: Flagelin, Fla-2, Fla-X, YIDX, bacteroides integrase, or a fragment or an equivalent of each thereof;
j) a systemic lupus erythematosus-relevant antigen and is derived from an antigen selected from one or more of the group: double-stranded (ds)DNA, ribonucleoprotein (RNP), Smith (Sm), Sjögren's-syndrome-related antigen A (SS-A)/Ro, Sjögren's-syndrome-related antigen B (SS-B)/La, RO60, RO52, histones, or a fragment or an equivalent of each thereof;
k) an atherosclerosis-relevant antigen and is derived from an antigen selected from one or more of the group: ApoB, ApoE or a fragment or an equivalent of each thereof;
l) a COPD-relvant antigen and/or emphysema-relevant antigen and is derived from elastin or a fragment or an equivalent thereof;
m) a psoriasis-relevant antigen and is derived from an antigen selected from one or more of the group: Cap18, ADMTSL5, ATL5, or a fragment or an equivalent of each thereof;
n) an autoimmune hepatitis-relevant antigen and is derived from an antigen selected from one or more of the group: CYP2D6, SLA, or a fragment or an equivalent of each thereof;
o) an uveitis-relevant antigen and is derived from arrestin or a fragment or an equivalent thereof;
p) a Sjogren's Syndrome-relevant antigen and is derived from an antigen selected from one or more of the group: (SS-A)/Ro, (SS-B)/La, MR3, RO60, RO52, or a fragment or an equivalent of each thereof;
q) a scleroderma-relevant antigen and is derived from an antigen selected from one or more of the group: CENP-C, TOP 1, RNA polymerase III, or a fragment or an equivalent of each thereof;
r) an anti-phospholipid syndrome-relevant antigen and is derived from APOH or a fragment or an equivalent thereof;
s) an ANCA-associated vasculitis-relevant antigen and is derived from an antigen selected from one or more of the group: MPO, PRTN3, or a fragment or an equivalent of each thereof; or
t) a Stiff Man Syndrome-relevant antigen and is derived from GAD or a fragment or an equivalent thereof.

102. The complex of any of claims 82-101, wherein the MHC protein of the pMHC complex comprises all or part of a classical MHC class I protein, non-classical MHC class I protein, classical MHC class II protein, non-classical MHC class II protein, MHC dimers (Fc fusions), MHC tetramers, or a polymeric form of a MHC protein, wherein the MHC protein optionally comprises a knob-in-hole based MHC-alpha-Fc/MHC-beta-Fc heterodimer or multimer.

103. The complex of any of claims 82-102, wherein the MHC protein of the pMHC complex comprises all or part of a polypeptide of the group: HLA DR, HLA DQ, HLA DP, HLA-A, HLA-B, HLA-C, HLA-E, HLA-F, HLA-G, CD1d, or a fragment or an equivalent of each thereof.

104. The complex of any of claims 82-103, wherein the MHC protein of the pMHC complex comprises all or part of a polypeptide of the group: HLA-DR, HLA-DQ, HLA-DP, or a fragment or an equivalent of each thereof.

105. The complex of any of claims 82-104, wherein the MHC protein of the pMHC complex comprises all or part of a polypeptide of the group: HLA-DRB1/DRA, HLA-DRB3/DRA, HLA-DRB4/DRA, HLA-DRB5/DRA, HLA-DQA1/HLA-DQB1, HLA-DPB1/HLA-DPA1, or a fragment or an equivalent of each thereof.

106. The complex of any of claims 82-85 and 88-105, wherein the pMHC complex comprises:
a) a diabetes-relevant antigen derived from an antigen selected from one or more of the group: hInsB₁₀₋₁₈, hIGRP₂₂₈₋₂₃₆, hIGRP₂₆₅₋₂₇₃, IGRP₂₀₆₋₂₁₄, hIGRP₂₀₆₋₂₁₄, NRP-A7, NRP-I4, NRP-V7, YAI/D^{b}, INS B₁₅₋₂₃, PPI_{76-90 (K88S)}, IGRP₁₃₋₂₅, GAD₅₅₅₋₅₆₇, GAD_{555-567(557I)}, IGRP₂₃₋₃₅, B₂₄-C₃₆, PPI₇₆₋₉₀, INS-I9, TUM, G6Pase, Pro-insulin_{L2-10}, Pro-insulin_{L3-11}, Pro-insulin_{L6-14}, Pro-insulin_{B5-14}, Pro-insulin₁₀₋₁₈, Pro-insulin_{B14-22}, Pro-insulin_{B15-24}, Pro-insulin_{B17-25}, Pro-insulin_{B18-27}, Pro-insulin_{B20-27}, Pro-insulin_{B21-29}, Pro-insulin_{B25-C1}, Pro-insulin_{B27-C5}, Pro-insulin_{C20-28}, Pro-insulin_{C25-33}, Pro-insulin_{C29-A5}, Pro-insulin_{A1-10}, Pro-insulin_{A2-10}, Pro-insulin_{A12-20}, or a fragment or an equivalent of each thereof;
b) a multiple sclerosis-relevant antigen derived from an antigen selected from one or more of the group: MOG₃₅₋₅₅, MOG₃₆₋₅₅, MAG₂₈₇₋₂₉₅, MAG₅₀₉₋₅₁₇, MAG₅₅₆₋₅₆₄, MBP₁₁₀₋₁₁₈, MOG₁₁₄₋₁₂₂, MOG₁₆₆₋₁₇₅, MOG₁₇₂₋₁₈₀, MOG₁₇₉₋₁₈₈, MOG₁₈₈₋₁₉₆, MOG₁₈₁₋₁₈₉, MOG₂₀₅₋₂₁₄, PLP₈₀₋₈₈, MAG₂₈₇₋₂₉₅, MAG₅₀₉₋₅₁₇, MAG₅₅₆₋₅₆₄, MOG₉₇₋₁₀₉ MOG_{97-109(E107S)}, MBP₈₉₋₁₀₁, PLP₁₇₅₋₁₉₂, PLP₉₄₋₁₀₈, MBP₈₆₋₉₈, PLP₅₄₋₆₈, PLP₂₄₉₋₂₆₃, MOG₁₅₆₋₁₇₀, MOG₂₀₁₋₂₁₅, MOG₃₈₋₅₂, MOG₂₀₃₋₂₁₇, PLP₂₅₀₋₂₆₄, MPB₁₃₋₃₂, MPB₈₃₋₉₉, MPB₁₁₁₋₁₂₉, MPB₁₄₆₋₁₇₀, MOG₂₂₃₋₂₃₇, MOG₆₋₂₀, PLP₈₈₋₁₀₂, PLP₁₃₉₋₁₅₄, or a fragment or an equivalent of each thereof;
c) a Celiac Disease-relevant antigen derived from an antigen selected from one or more of the group: aGlia₅₇₋₆₈, aGlia₆₂₋₇₂, aGlia₂₁₇₋₂₂₉, or a fragment or an equivalent of each thereof;
d) a primary biliary cirrhosis-relevant antigen derived from an antigen selected from one or more of the group: PDC-E2₁₂₂₋₁₃₅, PDC-E2₂₄₉₋₂₆₂, PDC-E2₂₄₉₋₂₆₃, PDC-E2₆₂₉₋₆₄₃, PDC-E₂₇₂₋₈₆, PDC-E2₃₅₃₋₃₆₇, PDC-E2₄₂₂₋₄₃₆, PDC-E2₆₂₉₋₆₄₃, PDC-E2₈₀₋₉₄, PDC-E2₃₅₃₋₃₆₇, PDC-E₂₅₃₅₋₅₄₉, or a fragment or an equivalent of each thereof;
e) a pemphigus folliaceus-relevant antigen and/or pemphigus vulgaris-relevant antigen, each of which is derived from an antigen selected from one or more of the group: DG1₂₁₆₋₂₂₉, DG3₉₇₋₁₁₁, DG3₂₅₁₋₂₆₅, DG3₄₄₁₋₄₅₅, DG3₃₅₁₋₃₆₅, DG3₄₅₃₋₄₆₇, DG3₅₄₀₋₅₅₄, DG3₂₈₀₋₂₉₄, DG3₃₂₆₋₃₄₀, DG3₃₆₇₋₃₈₁, DG3₁₃₋₂₇, DG3₃₂₃₋₃₃₇, DG3₄₃₈₋₄₅₂, DG1₄₈₋₆₂, DG1₂₀₆₋₂₂₂, DG1₃₆₃₋₃₇₇, DG1₃₋₁₇, DG1₁₉₂₋₂₀₆, DG1₃₂₆₋₃₄₀, DG1₁₋₁₅, DG1₃₅₋₄₉, DG1₃₂₅₋₃₃₉, or a fragment or an equivalent of each thereof;
f) a neuromyelitis optica spectrum disorder-relevant antigen derived from an antigen selected from one or more of the group: AQP4₁₂₉₋₁₄₃, AQP4₂₈₄₋₂₉₈, AQP4₆₃₋₇₆, AQP4₁₂₉₋₁₄₃, AQP4₃₉₋₅₃, or a fragment or an equivalent of each thereof;
g) an allergic asthma-relevant antigen derived from an antigen selected from one or more of the group: DERP1₁₆₋₃₀, DERP1₁₇₁₋₁₈₅, DERP1₁₁₀₋₁₂₄, DERP-2₂₆₋₄₀, DERP-2₁₀₇₋₁₂₁, or a fragment or an equivalent of each thereof;
h) an inflammatory bowel disease-relevant antigen derived from an antigen selected from one or more of the group: bacteroides integrase antigen₁₈₃₋₁₉₇, bacteroides integrase antigen₁₄₋₁₆₀, bacteroides integrase antigen₁₇₅₋₁₈₉, bacteroides integrase antigen₁₋₁₅, bacteroides integrase antigen₁₈₃₋₁₉₇, bacteroides integrase antigen₃₀₋₄₄, bacteroides integrase antigen₇₀₋₈₄, bacteroides integrase antigen₃₃₇₋₃₅₁, bacteroides integrase antigen₁₇₁₋₁₈₅, bacteroides integrase antigen₄₋₁₈, bacteroides integrase antigen₂₅₆₋₂₇₀, Fla-2/Fla-X₃₆₆₋₃₈₀, Fla-2/Fla-X₁₆₄₋₁₇₈, Fla-2/Fla-X₂₆₁₋₂₇₅, Fla-2/Fla-X₁₋₁₅, Fla-2/Fla-X₅₁₋₆₅, Fla-2/Fla-X₂₆₉₋₂₈₃, Fla-2/Fla-X₄₋₁₈, Fla-2/Fla-X₂₇₁₋₂₈₅, YIDX₇₈₋₉₂, YIDX₉₃₋₁₀₇, YIDX₉₈₋₁₁₂, YIDX₂₃₋₃₇, YIDX₇₈₋₉₂, YIDX₁₉₅₋₂₀₉, YIDX₂₂₋₃₆, YIDX₈₀₋₉₄, YIDX₁₀₁₋₁₁₅, or a fragment or an equivalent of each thereof;
i) a systemic lupus erythematosus-relevant antigen derived from an antigen selected from one or more of the group: H4₇₁₋₉₄, H4₇₄₋₈₈, H4₇₆₋₉₀, H4₇₅₋₈₉, H4₇₈₋₉₂, H4₈₀₋₉₄, H2B₁₀₋₂₄, H2B₁₆₋₃₀, H1′₂₂₋₄₂, H1′₂₇₋₄₁, or a fragment or an equivalent of each thereof;
j) an atherosclerosis-relevant antigen derived from an antigen selected from one or more of the group: ApoB₃₅₀₁₋₃₅₁₆, ApoB₁₉₅₂₋₁₉₆₆, ApoB₉₇₈₋₉₉₃, ApoB₃₄₉₈₋₃₅₁₃, ApoB_{210A}, ApoB_{210B}, ApoB_{210C}, or a fragment or an equivalent of each thereof;
k) a COPD-relvant antigen and/or emphysema-relevant antigen, each of which is derived from an antigen selected from one or more of the group: elastin₈₉₋₁₀₃, elastin₆₉₈₋₇₁₂, elastin₈₋₂₂, elastin₉₄₋₁₀₈, elastin₁₃₋₂₇, elastin₆₉₅₋₇₀₉, elastin₅₆₃₋₅₇₇, elastin₅₅₈₋₅₇₂, elastin₆₉₈₋₇₁₂, elastin₅₆₆₋₅₈₀, elastin₅₄₅₋₆₅₉, or a fragment or an equivalent of each thereof;
l) a psoriasis-relevant antigen derived from an antigen selected from one or more of the group: Cap18₆₄₋₇₈, Cap18₃₄₋₄₈, Cap18₄₇₋₆₁, Cap18₁₅₁₋₁₆₅, Cap18₁₄₉₋₁₆₃, Cap18₁₅₂₋₁₆₆, Cap18₁₃₁₋₁₄₅, Cap₁₈₂₄₋₃₈, ADMTSL5245₋₂₅₉, ADMTSL5₂₆₇₋₂₈₁, ADMTSL5₃₇₂₋₃₈₆, ADMTSL5₂₈₉₋₃₀₃, ADMTSL5₃₉₆₋₄₁₀, ADMTSL5₄₃₃₋₄₄₇, ADMTSL5₁₄₂₋₁₅₆, ADMTSL5₂₃₆₋₂₅₀, ADMTSL5₃₀₁₋₃₁₅, ADMTSL5₂₀₃₋₂₁₇, ADMTSL5₄₀₄₋₄₁₈, or a fragment or an equivalent of each thereof;
m) an autoimmune hepatitis-relevant antigen derived from an antigen selected from one or more of the group: (CYP2D6)₁₉₃₋₂₀₇, CYP2D6₇₆₋₉₀, CYP2D6₂₉₃₋₃₀₇, CYP2D6₃₁₃₋₃₃₂, CYP2D6₃₉₃₋₄₁₂, CYP2D6₁₉₉₋₂₁₃, CYP2D6₄₅₀₋₄₆₄, CYP2D6₃₀₁₋₃₁₅, CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₃₁₋₁₄₅, CYP2D6₂₁₆₋₂₃₀, CYP2D6₂₃₈₋₂₅₂, CYP2D6₁₉₉₋₂₁₃, CYP2D6₂₃₅₋₂₅₂, CYP2D6₂₉₃₋₃₀₇, CYP2D6₃₈₁₋₃₉₅, CYP2D6₄₂₉₋₄₄₃, SLA₃₃₄₋₃₄₈, SLA₁₉₆₋₂₁₀, SLA₁₁₅₋₁₂₉, SLA₃₇₃₋₃₈₆, SLA₁₈₆₋₁₉₇, SLA₃₁₇₋₃₃₁, SLA₁₇₁₋₁₈₅, SLA₄₁₇₋₄₃₁, SLA₃₅₉₋₃₇₃, SLA₂₁₅₋₂₂₉, SLA₁₁₁₋₁₂₅, SLA₁₁₀₋₁₂₄, SLA₂₉₉₋₃₁₃, SLA₃₄₂₋₃₅₆, SLA₄₉₋₆₃, SLA₁₁₉₋₁₃₃, SLA₂₆₀₋₂₇₄, SLA₂₆₋₄₀, SLA₈₆₋₁₀₀, SLA₃₃₁₋₃₄₅, or a fragment or an equivalent of each thereof;
n) an uveitis-relevant antigen derived from an antigen selected from one or more of the group: arrestin₁₉₉₋₂₁₃, arrestin₇₇₋₉₁, arrestin₂₅₀₋₂₆₄, arrestin₁₇₂₋₁₈₆, arrestin₃₅₄₋₃₆₈, arrestin₂₃₉₋₂₅₃, arrestin₁₀₂₋₁₁₆, arrestin₅₉₋₇₃, arrestin₂₈₀₋₂₉₄, arrestin₂₉₁₋₃₀₆, arrestin₁₉₅₋₂₀₉, arrestin₂₀₀₋₂₁₄, or a fragment or an equivalent of each thereof;
o) a Sjogren's Syndrome-relevant antigen derived from an antigen selected from one or more of the group: RO60₁₂₇₋₁₄₁, RO60₅₂₃₋₅₃₇, RO60₂₄₃₋₂₅₇, RO60₄₈₄₋₄₉₈, RO60₃₄₇₋₃₆₁, RO60₃₆₉₋₃₈₃, RO60₄₂₆₋₄₄₀, RO60₂₆₇₋₂₈₁, RO60₁₇₈₋₁₉₂, RO60₃₅₈₋₃₇₂, RO60₂₂₁₋₂₃₅, RO60₃₁₈₋₃₃₂, RO60₄₀₇₋₄₂₁, RO60₄₅₉₋₄₇₃, RO60₅₁₋₆₅, RO60₃₁₂₋₃₂₆, LA₂₄₁₋₂₅₅, LA₁₀₁₋₁₁₅, LA₁₅₃₋₁₆₇, LA₁₇₈₋₁₉₂, LA₁₉₋₃₃, LA₃₇₋₅₁, LA₁₃₃₋₁₄₇, LA₅₀₋₆₄, LA₃₂₋₄₆, LA₁₅₃₋₁₆₇, LA₈₃₋₉₇, LA₁₃₆₋₁₅₀, LA₂₉₇₋₃₁₁, LA₅₉₋₇₃, LA₁₅₁₋₁₆₅, LA₈₆₋₁₀₀, LA₁₅₄₋₁₆₈, or a fragment or an equivalent of each thereof;
p) a scleroderma-relevant antigen derived from an antigen selected from one or more of the group: TOP1₃₄₆₋₃₆₀, TOP1₄₂₀₋₄₃₄, TOP1₇₅₀₋₇₆₄, TOP1₄₁₉₋₄₃₃, TOP1₅₉₁₋₆₀₅, TOP1₆₉₅₋₇₀₉, TOP1₃₀₅₋₃₁₉, TOP1₃₄₆₋₃₆₀, TOP1₄₁₉₋₄₃₃, TOP1₄₂₅₋₄₃₉, TOP1₆₁₄₋₆₂₈, CENP-C₂₉₇₋₃₁₁, CENP-C₈₅₇₋₈₇₁, CENP-C₈₈₇₋₉₀₁, CENP-C₂₁₂₋₂₂₆, CENP-C₆₄₃₋₆₅₇, CENP-C₈₃₂₋₈₄₆, CENP-C₁₆₇₋₁₈₁, CENP-C₂₄₆₋₂₆₀, CENP-C₈₄₆₋₈₆₀, CENP-C₁₄₉₋₁₆₃, CENP-C₈₃₃₋₈₄₇, CENP-C₈₄₇₋₈₆₁, or a fragment or an equivalent of each thereof;
q) an anti-phospholipid syndrome-relevant antigen derived from an antigen selected from one or more of the group: APOH₂₃₅₋₂₄₉, APOH₃₀₆₋₃₂₀, APOH₂₃₇₋₂₅₁, APOH₂₉₅₋₃₀₉, APOH₂₈₄₂, APOH₁₇₃₋₁₈₇, APOH₂₆₄₋₂₇₈, APOH₂₉₅₋₃₀₉, APOH₄₉₋₆₃, APOH₂₆₉₋₂₈₃, APOH₂₉₅₋₃₀₉, APOH₃₂₁₋₃₅₅, APOH₃₂₂₋₃₃₆, APOH₃₂₄₋₃₃₈, or a fragment or an equivalent of each thereof;
r) an ANCA-associated vasculitis-relevant antigen derived from an antigen selected from one or more of the group: MPO₅₀₆₋₅₂₀, MPO₃₀₂₋₃₁₆, MPO₇₋₂₁, MPO₆₈₉₋₇₀₃, MPO₂₄₈₋₂₆₂, MPO₄₄₄₋₄₅₈, MPO₅₁₃₋₅₂₇, MPO₉₇₋₁₁₁, MPO₆₁₆₋₆₃₀, MPO₄₆₂₋₄₇₆, MPO₆₁₇₋₆₃₁, MPO₇₁₄₋₇₂₈, PRTN3₄₄₋₅₈, PRTN3₂₃₄₋₂₄₈, PRTN3₅₉₋₇₃, PRTN3₁₁₇₋₁₃₁, PRTN3₁₆₄₋₁₇₈, PRTN3₇₁₋₈₅, PRTN3₂₄₁₋₂₅₅, PRTN3₅₉₋₇₃, PRTN3₁₈₃₋₁₉₇, PRTN3₆₂₋₇₆, PRTN3₁₁₈₋₁₃₂, PRTN3₂₃₉₋₂₅₃, or a fragment or an equivalent of each thereof; or
s) a Stiff Man Syndrome-relevant antigen derived from an antigen selected from one or more of the group: GAD₂₁₂₋₂₂₆, GAD₅₅₅₋₅₆₉, GAD₂₉₇₋₃₁₁, or a fragment or an equivalent of each thereof.

107. The complex of any of claims 82-85 and 88-106, wherein the pMHC complex comprises:
a) a diabetes-relevant antigen derived from an antigen selected from one or more of the group: hInsB₁₀₋₁₈, hIGRP₂₂₈₋₂₃₆, hIGRP₂₆₅₋₂₇₃, IGRP₂₀₆₋₂₁₄, hIGRP₂₀₆₋₂₁₄, NRP-A7, NRP-I4, NRP-V7, YAI/D^{b}, INS B₁₅₋₂₃, PPI_{76-90 (K88S)}, IGRP₁₃₋₂₅, GAD₅₅₅₋₅₆₇, GAD_{555-567(557I)}, IGRP₂₃₋₃₅, B₂₄-C₃₆, PPI₇₆₋₉₀, INS-I9, TUM, G6Pase, Pro-insulin_{L2-10}, Pro-insulin_{L3-11}, Pro-insulin_{L6-14}, Pro-insulin_{B5-14}, Pro-insulin_{B10-18}, Pro-insulin_{B14-22}, Pro-insulin_{B15-24}, Pro-insulin_{B17-25}, Pro-insulin_{B18-27}, Pro-insulin_{B20-27}, Pro-insulin_{B21-29}, Pro-insulin_{B25}-C₁, Pro-insulin_{B27-C5}, Pro-insulin_{C20-28}, Pro-insulin_{C25-33}, Pro-insulin_{C29-A5}, Pro-insulin_{A1-10}, Pro-insulin_{A2-10}, Pro-insulin_{A12-20}, or a fragment or an equivalent of each thereof, and the MHC protein of the pMHC complex comprises all or part of HLA-DR or a fragment or an equivalent thereof;
b) a multiple sclerosis-relevant antigen derived from an antigen selected from one or more of the group: MOG₃₅₋₅₅, MOG₃₆₋₅₅, MAG₂₈₇₋₂₉₅, MAG₅₀₉₋₅₁₇, MAG₅₅₆₋₅₆₄, MBP₁₁₀₋₁₁₈, MOG₁₁₄₋₁₂₂, MOG₁₆₆₋₁₇₅, MOG₁₇₂₋₁₈₀, MOG₁₇₉₋₁₈₈, MOG₁₈₈₋₁₉₆, MOG₁₈₁₋₁₈₉, MOG₂₀₅₋₂₁₄, PLP₈₀₋₈₈, MAG₂₈₇₋₂₉₅, MAG₅₀₉₋₅₁₇, MAG₅₅₆₋₅₆₄, MOG₉₇₋₁₀₉ MOG_{97-109(E107S)}, MBP₉₋₁₀₁, PLP₁₇₅₋₁₉₂, PLP₉₄₋₁₀₈, MBP₈₆₋₉₈, PLP₅₄₋₆₈, PLP₂₄₉₋₂₆₃, MOG₁₅₆₋₁₇₀, MOG₂₀₁₋₂₁₅, MOG₃₈₋₅₂, MOG₂₀₃₋₂₁₇, PLP₂₅₀₋₂₆₄, MPB₁₃₋₃₂, MPB₈₃₋₉₉, MPB₁₁₁₋₁₂₉, MPB₁₄₆₋₁₇₀, MOG₂₂₃₋₂₃₇, MOG₆₋₂₀, PLP₈₈₋₁₀₂, PLP₁₃₉₋₁₅₄, or a fragment or an equivalent of each thereof, and the MHC protein of the pMHC complex comprises all or part of HLA-DR or a fragment or an equivalent thereof;
c) a Celiac Disease-relevant antigen derived from an antigen selected from one or more of the group: aGlia₅₇₋₆₈, aGlia₆₂₋₇₂, aGlia₂₁₇₋₂₂₉, or a fragment or an equivalent of each thereof, and the MHC protein of the pMHC complex comprises all or part of HLA-DQ or a fragment or an equivalent thereof;
d) a primary biliary cirrhosis-relevant antigen derived from an antigen selected from one or more of the group: PDC-E2₁₂₂₋₁₃₅, PDC-E2₂₄₉₋₂₆₂, PDC-E2₂₄₉₋₂₆₃, PDC-E2₆₂₉₋₆₄₃, PDC-E₂₇₂₋₈₆, PDC-E2₃₅₃₋₃₆₇, PDC-E2₄₂₂₋₄₃₆, PDC-E2₆₂₉₋₆₄₃, PDC-E2₈₀₋₉₄, PDC-E2₃₅₃₋₃₆₇, PDC-E₂₅₃₅₋₅₄₉, or a fragment or an equivalent of each thereof, and the MHC protein of the pMHC complex comprises all or part of HLA-DR or a fragment of an equivalent thereof;
e) a pemphigus folliaceus-relevant antigen and/or pemphigus vulgaris-relevant antigen, each of which is derived from an antigen selected from one or more of the group: DG1₂₁₆₋₂₂₉, DG3₉₇₋₁₁₁, DG3₂₅₁₋₂₆₅, DG3₄₄₁₋₄₅₅, DG3₃₅₁₋₃₆₅, DG3₄₅₃₋₄₆₇, DG3₅₄₀₋₅₅₄, DG3₂₈₀₋₂₉₄, DG3₃₂₆₋₃₄₀, DG3₃₆₇₋₃₈₁, DG3₁₃₋₂₇, DG3₃₂₃₋₃₃₇, DG3₄₃₈₋₄₅₂, DG1₄₈₋₆₂, DG1₂₀₆₋₂₂₂, DG1₃₆₃₋₃₇₇, DG1₃₋₁₇, DG1₁₉₂₋₂₀₆, DG1₃₂₆₋₃₄₀, DG1₁₋₁₅, DG1₃₅₋₄₉, DG1₃₂₅₋₃₃₉, or a fragment or an equivalent of each thereof, and the MHC protein of the pMHC complex comprises all or part of HLA-DR or a fragment or an equivalent thereof;
f) a neuromyelitis optica spectrum disorder-relevant antigen derived from an antigen selected from one or more of the group: AQP4₁₂₉₋₁₄₃, AQP4₂₈₄₋₂₉₈, AQP4₆₃₋₇₆, AQP4₁₂₉₋₁₄₃, AQP4₃₉₋₅₃, or a fragment or an equivalent of each thereof, and the MHC protein of the pMHC complex comprises all or part of HLA-DR or a fragment or an equivalent thereof;
g) an allergic asthma-relevant antigen derived from an antigen selected from one or more of the group: DERP1₁₆₋₃₀, DERP1₁₇₁₋₁₈₅, DERP1₁₁₀₋₁₂₄, DERP-2₂₆₋₄₀, DERP-2₁₀₇₋₁₂₁, or a fragment or an equivalent of each thereof, and the MHC protein of the pMHC complex comprises all or part of a polypeptide of the group: HLA-DR, HLA-DP, or a fragment or an equivalent of each thereof;
h) an inflammatory bowel disease-relevant antigen derived from an antigen selected from one or more of the group: bacteroides integrase antigen₁₈₃₋₁₉₇, bacteroides integrase antigen₁₄₋₁₆₀, bacteroides integrase antigen₁₇₅₋₁₈₉, bacteroides integrase antigen₁₋₁₅, bacteroides integrase antigen₁₈₃₋₁₉₇, bacteroides integrase antigen₃₀₋₄₄, bacteroides integrase antigen₇₀₋₈₄, bacteroides integrase antigen₃₃₇₋₃₅₁, bacteroides integrase antigen₁₇₁₋₁₈₅, bacteroides integrase antigen₄₋₁₈, bacteroides integrase antigen₂₅₆₋₂₇₀, Fla-2/Fla-X₃₆₆₋₃₈₀, Fla-2/Fla-X₁₆₄₋₁₇₈, Fla-2/Fla-X₂₆₁₋₂₇₅, Fla-2/Fla-X₁₋₁₅, Fla-2/Fla-X₅₁₋₆₅, Fla-2/Fla-X₂₆₉₋₂₈₃, Fla-2/Fla-X₄₋₁₈, Fla-2/Fla-X₂₇₁₋₂₈₅, YIDX₇₈₋₉₂, YIDX₉₃₋₁₀₇, YIDX₉₈₋₁₁₂, YIDX₂₃₋₃₇, YIDX₇₈₋₉₂, YIDX₁₉₅₋₂₀₉, YIDX₂₂₋₃₆, YIDX₈₀₋₉₄, YIDX₁₀₁₋₁₁₅, or a fragment or an equivalent of each thereof, and the MHC protein of the pMHC complex comprises all or part of HLA-DR or a fragment or an equivalent thereof;
i) a systemic lupus erythematosus-relevant antigen derived from an antigen selected from one or more of the group: H4₇₁₋₉₄, H4₇₄₋₈₈, H4₇₆₋₉₀, H4₇₅₋₈₉, H4₇₈₋₉₂, H4₈₀₋₉₄, H2B₁₀₋₂₄, H2B₁₆₋₃₀, H1′₂₂₋₄₂, H1′₂₇₋₄₁, or a fragment or an equivalent of each thereof, and the MHC protein of the pMHC complex comprises all or part of a polypeptide of the group: I-A_{d}, HLA-DR, or a fragment or an equivalent of each thereof;
j) an atherosclerosis-relevant antigen derived from an antigen selected from one or more of the group: ApoB₃₅₀₁₋₃₅₁₆, ApoB₁₉₅₂₋₁₉₆₆, ApoB₉₇₈₋₉₉₃, ApoB₃₄₉₈₋₃₅₁₃, ApoB_{210A}, ApoB_{210B}, ApoB_{210C}, or a fragment or an equivalent of each thereof, and the MHC protein of the pMHC complex comprises all or part of I-A_{b} or a fragment or an equivalent thereof;
k) a COPD-relvant antigen and/or emphysema-relevant antigen, each of which is derived from an antigen selected from one or more of the group: elastin₈₉₋₁₀₃, elastin₆₉₈₋₇₁₂, elastin₈₋₂₂, elastin₉₄₋₁₀₈, elastin₁₃₋₂₇, elastin₆₉₅₋₇₀₉, elastin₅₆₃₋₅₇₇, elastin₅₅₈₋₅₇₂, elastin₆₉₈₋₇₁₂, elastin₅₆₆₋₅₈₀, elastin₅₄₅₋₆₅₉, or a fragment or an equivalent of each thereof, and the MHC protein of the pMHC complex comprises all or part of HLA-DR or a fragment or an equivalent thereof;
l) a psoriasis-relevant antigen derived from an antigen selected from one or more of the group: Cap18₆₄₋₇₈, Cap18₃₄₋₄₈, Cap18₄₇₋₆₁, Cap18₁₅₁₋₁₆₅, Cap18₁₄₉₋₁₆₃, Cap18₁₅₂₋₁₆₆, Cap18₁₃₁₋₁₄₅, Cap18₂₄₋₃₈, ADMTSL5245₋₂₅₉, ADMTSL5₂₆₇₋₂₈₁, ADMTSL5₃₇₂₋₃₈₆, ADMTSL5₂₈₉₋₃₀₃, ADMTSL5₃₉₆₋₄₁₀, ADMTSL5₄₃₃₋₄₄₇, ADMTSL5₁₄₂₋₁₅₆, ADMTSL5₂₃₆₋₂₅₀, ADMTSL5₃₀₁₋₃₁₅, ADMTSL5₂₀₃₋₂₁₇, ADMTSL5₄₀₄₋₄₁₈, or a fragment or an equivalent of each thereof, and the MHC protein of the pMHC complex comprises all or part of HLA-DR or a fragment or an equivalent thereof;
m) an autoimmune hepatitis-relevant antigen derived from an antigen selected from one or more of the group: CYP2D6₁₉₃₋₂₀₇, CYP2D6₇₆₋₉₀, CYP2D6₂₉₃₋₃₀₇, CYP2D6₃₁₃₋₃₃₂, CYP2D6₃₉₃₋₄₁₂, CYP2D6₁₉₉₋₂₁₃, CYP2D6₄₅₀₋₄₆₄, CYP2D6₃₀₁₋₃₁₅, CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₃₁₋₁₄₅, CYP2D6₂₁₆₋₂₃₀, CYP2D6₂₃₈₋₂₅₂, CYP2D6₁₉₉₋₂₁₃, CYP2D6₂₃₅₋₂₅₂, CYP2D6₂₉₃₋₃₀₇, CYP2D6₃₈₁₋₃₉₅, CYP2D6₄₂₉₋₄₄₃, SLA₃₃₄₋₃₄₈, SLA₁₉₆₋₂₁₀, SLA₁₁₅₋₁₂₉, SLA₃₇₃₋₃₈₆, SLA₁₈₆₋₁₉₇, SLA₃₁₇₋₃₃₁, SLA₁₇₁₋₁₈₅, SLA₄₁₇₋₄₃₁, SLA₃₅₉₋₃₇₃, SLA₂₁₅₋₂₂₉, SLA₁₁₁₋₁₂₅, SLA₁₁₀₋₁₂₄, SLA₂₉₉₋₃₁₃, SLA₃₄₂₋₃₅₆, SLA₄₉₋₆₃, SLA₁₁₉₋₁₃₃, SLA₂₆₀₋₂₇₄, SLA₂₆₋₄₀, SLA₈₆₋₁₀₀, SLA₃₃₁₋₃₄₅, or a fragment or an equivalent of each thereof, and the MHC protein of the pMHC complex comprises all or part of HLA-DR or a fragment or an equivalent thereof;
n) an uveitis-relevant antigen derived from an antigen selected from one or more of the group: arrestin₁₉₉₋₂₁₃, arrestin₇₇₋₉₁, arrestin₂₅₀₋₂₆₄, arrestin₁₇₂₋₁₈₆, arrestin₃₅₄₋₃₆₈, arrestin₂₃₉₋₂₅₃, arrestin₁₀₂₋₁₁₆, arrestin₅₉₋₇₃, arrestin₂₈₀₋₂₉₄, arrestin₂₉₁₋₃₀₆, arrestin₁₉₅₋₂₀₉, arrestin₂₀₀₋₂₁₄, or a fragment or an equivalent of each thereof, and the MHC protein of the pMHC complex comprises all or part of HLA-DR or a fragment or an equivalent thereof;
o) a Sjogren's Syndrome-relevant antigen derived from an antigen selected from one or more of the group: RO60₁₂₇₋₁₄₁, RO60₅₂₃₋₅₃₇, RO60₂₄₃₋₂₅₇, RO60₄₈₄₋₄₉₈, RO60₃₄₇₋₃₆₁, RO60₃₆₉₋₃₈₃, RO60₄₂₆₋₄₄₀, RO60₂₆₇₋₂₈₁, RO60₁₇₈₋₁₉₂, RO60₃₅₈₋₃₇₂, RO60₂₂₁₋₂₃₅, RO60₃₁₈₋₃₃₂, RO60₄₀₇₋₄₂₁, RO60₄₅₉₋₄₇₃, RO60₅₁₋₆₅, RO60₃₁₂₋₃₂₆, LA₂₄₁₋₂₅₅, LA₁₀₁₋₁₁₅, LA₁₅₃₋₁₆₇, LA₁₇₈₋₁₉₂, LA₁₉₋₃₃, LA₃₇₋₅₁, LA₁₃₃₋₁₄₇, LA₅₀₋₆₄, LA₃₂₋₄₆, LA₁₅₃₋₁₆₇, LA₈₃₋₉₇, LA₁₃₆₋₁₅₀, LA₂₉₇₋₃₁₁, LA₅₉₋₇₃, LA₁₅₁₋₁₆₅, LA₈₆₋₁₀₀, LA₁₅₄₋₁₆₈, or a fragment or an equivalent of each thereof, and the MHC protein of the pMHC complex comprises all or part of a polypeptide of the group: HLA-DR, HLA-DP, or a fragment or an equivalent of each thereof;
p) a scleroderma-relevant antigen derived from an antigen selected from one or more of the group: TOP1₃₄₆₋₃₆₀, TOP1₄₂₀₋₄₃₄, TOP1₇₅₀₋₇₆₄, TOP1₄₁₉₋₄₃₃, TOP1₅₉₁₋₆₀₅, TOP1₆₉₅₋₇₀₉, TOP1₃₀₅₋₃₁₉, TOP1₃₄₆₋₃₆₀, TOP1₄₁₉₋₄₃₃, TOP1₄₂₅₋₄₃₉, TOP1₆₁₄₋₆₂₈, CENP-C₂₉₇₋₃₁₁, CENP-C₈₅₇₋₈₇₁, CENP-C₈₈₇₋₉₀₁, CENP-C₂₁₂₋₂₂₆, CENP-C₆₄₃₋₈₅₇, CENP-C₈₃₂₋₈₄₆, CENP-C₁₆₇₋₁₈₁, CENP-C₂₄₆₋₂₆₀, CENP-C₈₄₆₋₈₆₀, CENP-C₁₄₉₋₁₆₃, CENP-C₈₃₃₋₈₄₇, CENP-C₈₄₇₋₈₆₁, or a fragment or an equivalent of each thereof, and the MHC protein of the pMHC complex comprises all or part of HLA-DR or a fragment or an equivalent thereof;
q) an anti-phospholipid syndrome-relevant antigen derived from an antigen selected from one or more of the group: APOH₂₃₅₋₂₄₉, APOH₃₀₆₋₃₂₀, APOH₂₃₇₋₂₅₁, APOH₂₉₅₋₃₀₉, APOH₂₈₄₂, APOH₁₇₃₋₁₈₇, APOH₂₆₄₋₂₇₈, APOH₂₉₅₋₃₀₉, APOH₄₉₋₆₃, APOH₂₆₉₋₂₈₃, APOH₂₉₅₋₃₀₉, APOH₃₂₁₋₃₅₅, APOH₃₂₂₋₃₃₆, APOH₃₂₄₋₃₃₈, or a fragment or an equivalent of each thereof, and the MHC protein of the pMHC complex comprises all or part of HLA-DR or a fragment or an equivalent thereof;
r) an ANCA-associated vasculitis-relevant antigen derived from an antigen selected from one or more of the group: MPO₅₀₆₋₅₂₀, MPO₃₀₂₋₃₁₆, MPO₇₋₂₁, MPO₆₈₉₋₇₀₃, MPO₂₄₈₋₂₆₂, MPO₄₄₄₋₄₅₈, MPO₅₁₃₋₅₂₇, MPO₉₇₋₁₁₁, MPO₆₁₆₋₆₃₀, MPO₄₆₂₋₄₇₆, MPO₆₁₇₋₆₃₁, MPO₇₁₄₋₇₂₈, PRTN3₄₄₋₅₈, PRTN3₂₃₄₋₂₄₈, PRTN3₅₉₋₇₃, PRTN3₁₁₇₋₁₃₁, PRTN3₁₆₄₋₁₇₈, PRTN3₇₁₋₈₅, PRTN3₂₄₁₋₂₅₅, PRTN3₅₉₋₇₃, PRTN3₁₈₃₋₁₉₇, PRTN3₆₂₋₇₆, PRTN3₁₁₈₋₁₃₂, PRTN3₂₃₉₋₂₅₃, or a fragment or an equivalent of each thereof, and the MHC protein of the pMHC complex comprises all or part of HLA-DR or a fragment or an equivalent thereof; or
s) a Stiff Man Syndrome-relevant antigen derived from an antigen selected from one or more of the group: GAD₂₁₂₋₂₂₆, GAD₅₅₅₋₅₆₉, GAD₂₉₇₋₃₁₁, and the MHC protein of the pMHC complex comprises all or part of a polypeptide of the group: HLA-DR, HLA-DQ, or a fragment or an equivalent of each thereof.

108. The complex of any of claims 82-85 and 88-107, wherein the pMHC complex is for the treatment of:
a) type I diabetes and the pMHC complex is selected from the group of: PPI_{76-90(K88S)}-HLA-DRB1*0401/DRA, IGRP₁₃₋₂₅-HLA-DRB1*0301/DRA, GAD₅₅₅₋₅₆₇ HLA-DRB1*0401/DRA, GAD_{555-567(557I)}-HLA-DRB1*0401/DRA, IGRP₂₃₋₃₅-HLA-DRB1*0401/DRA, B₂₄-C₃₆-HLA-DRB1*0301/DRA, or PPI₇₆₋₉₀-HLA-DRB1*0401/DRA;
b) multiple sclerosis and the pMHC complex is selected from the group of: MBP₈₅₆₋₉₈-HLA-DRB1*1501/DRA, MBP₈₉₋₁₀₁-HLA-DRB5*0101/DRA, MOG₃₈₋₅₂-HLA-DRB4*0101/DRA, MOG_{97-109(E107S)}-HLA-DRB1*0401/DRA, MOG₂₀₃₋₂₁₇-HLA-DRB3*0101/DRA, PLP₅₄₋₆₈-HLA-DRB3*0101/DRA, PLP₉₄₋₁₀₈-HLA-DRB1*0301/DRA, PLP₂₅₀₋₂₆₄-HLA-DRB4*0101/DRA, MPB₁₃₋₃₂-HLA-DRB5*0101/DRA, MPB₈₃₋₉₉-HLA-DRB5*0101/DRA, MPB₁₁₁₋₁₂₉-HLA-DRB5*0101/DRA, MPB₁₄₆₋₁₇₀-HLA-DRB5*0101/DRA, MOG₂₂₃₋₂₃₇-HLA-DRB3*0202/DRA, MOG₆₋₂₀-HLA-DRB5*0101/DRA, PLP₈₈₋₁₀₂-HLA-DRB3*0202/DRA, or PLP₁₃₉₋₁₅₄-HLA-DRB5*0101/DRA;
c) Celiac Disease and the pMHC complex is selected from the group of: aGlia₅₇₋₆₈-HLA-DQA1*0501/HLA-DQB1*0201, aGlia₆₂₋₇₂-HLA-DQA1*0501/HLA-DQB1*0201, aGlia₂₁₇₋₂₂₉-HLA-DQA1*0501/HLA-DQB1*0302, or aGlia₂₁₇₋₂₂₉-HLA-DQA1*03/HLA-DQB1*0302;
d) primary biliary cirrhosis and the pMHC complex is selected from the group of: PDC-E2₁₂₂₋₁₃₅-HLA-DRB4*0101/DRA, PDC-E2₂₄₉₋₂₆₂-HLA-DRB4*0101/DRA, PDC-E2₂₄₉₋₂₆₃-HLA-DRB1*0801/DRA, PDC-E2₆₂₉₋₆₄₃-HLA-DRB1*0801/DRA, PDC-E2₇₂₋₈₆-HLA-DRB3*0202/DRA, PDC-E2₃₅₃₋₃₆₇-HLA-DRB3*0202/DRA, PDC-E2₄₂₂₋₄₃₆-HLA-DRB3*0202/DRA, PDC-E2₆₂₉₋₆₄₃-HLA-DRB4*0101/DRA, PDC-E2₈₀₋₉₄-HLA-DRB5*0101/DRA, PDC-E2₃₅₃₋₃₆₇-HLA-DRB5*0101/DRA, or PDC-E2₅₃₅₋₅₄₉-HLA-DRB5*0101/DRA, mPDC-E2₁₆₆₋₅₁₁-I-A_{g7}, or mPDC-E2₈₂₋₉₆-I-A_{g7};
e) pemphigus folliaceus and/or pemphigus vulgaris and the pMHC complex is selected from the group of: DG1₂₁₆₋₂₂₉-HLA-DRB1*0101/DRA, DG1₂₁₆₋₂₂₉-HLA-DRB1*0102/DRA, DG3₉₇₋₁₁₁-HLA-DRB1*0402/DRA, DG3₂₅₁₋₂₆₅-HLA-DRB1*0402/DRA, DG3₂₅₁₋₂₆₅-HLA-DRB1*0401/DRA, DG3₄₄₁₋₄₅₅-HLA-DRB1*0402/DRA, DG3₃₅₁₋₃₆₅-HLA-DRB3*0202/DRA, DG3₄₅₃₋₄₆₇-HLA-DRB3*0202/DRA, DG3₅₄₀₋₅₅₄-HLA-DRB3*0202/DRA, DG3₂₈₀₋₂₉₄-HLA-DRB4*0101/DRA, DG3₃₂₆₋₃₄₀-HLA-DRB4*0101/DRA, DG3₃₆₇₋₃₈₁-HLA-DRB4*0101/DRA, DG3₁₃₋₂₇-HLA-DRB5*0101/DRA, DG3₃₂₃₋₃₃₇-HLA-DRB5*0101/DRA, DG3₄₃₈₋₄₅₂-HLA-DRB5*0101/DRA, DG1₄₈₋₆₂-HLA-DRB3*0202/DRA, DG1₂₀₆₋₂₂₂-HLA-DRB3*0202/DRA, DG1₃₆₃₋₃₇₇-HLA-DRB3*0202/DRA, DG1₃₋₁₇-HLA-DRB4*0101/DRA, DG1₁₉₂₋₂₀₆-HLA-DRB4*0101/DRA, DG1₃₂₆₋₃₄₀-HLA-DRB4*0101/DRA, DG1₁₋₁₅-HLA-DRB5*0101/DRA, DG1₃₅₋₄₉-HLA-DRB5*0101/DRA, or DG1₃₂₅₋₃₃₉-HLA-DRB5*0101/DRA;
f) neuromyelitis optica spectrum disorder and the pMHC complex is selected from the group of: AQP4₁₂₉₋₁₄₃-HLA-DRB1*0101/DRA, AQP4₂₈₄₋₂₉₈-HLA-DRB1*0301/DRA, AQP4₆₃₋₇₆-HLA-DRB1*0301/DRA, AQP4₁₂₉₋₁₄₃-HLA-DRB1*0401/DRA, or AQP4₃₉₋₅₃-HLA-DRB1*1501/DRA;
g) allergic asthma and the pMHC complex is selected from the group of: DERP-1₁₆₋₃₀-HLA-DRB1*0101/DRA, DERP-1₁₆₋₃₀-HLA-DRB1*1501/DRA, DERP₁₁₇₁₋₁₈₅-HLA-DRB1*1501/DRA, DERP-1₁₁₀₋₁₂₄-HLA-DPB1*0401/DRA, DERP-2₂₆₋₄₀-HLA-DRB1*0101/DRA; DERP-2₂₆₋₄₀-HLA-DRB1*1501/DRA, or DERP-2₁₀₇₋₁₂₁-HLA-DRB1*0301/DRA;
h) inflammatory bowel disease and the pMHC complex is selected from the group of: bacteroides integrase antigen₁₈₃₋₁₉₇-HLA-DRB3*0101/DRA, bacteroides integrase antigen₁₄₆₋₁₆₀-HLA-DRB3*0101/DRA, bacteroides integrase antigen₁₇₅₋₁₈₉-HLA-DRB3*0101/DRA, bacteroides integrase antigen₁₋₁₅-HLA-DRB5*0101/DRA, bacteroides integrase antigen₁₈₃₋₁₉₇-HLA-DRB5*0101/DRA, bacteroides integrase antigen₁₈₃₋₁₉₇-HLA-DRB3*0101/DRA, bacteroides integrase antigen₃₀₋₄₄-HLA-DRB5*0101/DRA, bacteroides integrase antigen₇₀₋₈₄-HLA-DRB4*0101/DRA, bacteroides integrase antigen₃₃₇₋₃₅₁-HLA-DRB4*0101/DRA, bacteroides integrase antigen₁₇₁₋₁₈₅-HLA-DRB4*0101/DRA, bacteroides integrase antigen₄₋₁₈-HLA-DRB3*0202/DRA, bacteroides integrase antigen₁₇₁₋₁₈₅-HLA-DRB3*0202/DRA, bacteroides integrase antigen₂₅₆₋₂₇₀-HLA-DRB3*0202/DRA, Fla-2/Fla-X₃₆₆₋₃₈₀-HLA-DRB3*0101/DRA, Fla-2/Fla-X₁₆₄₋₁₇₈-HLA-DRB3*0101/DRA, Fla-2/Fla-X₂₆₁₋₂₇₅-HLA-DRB5*0101/DRA, Fla-2/Fla-X₁₋₁₈-HLA-DRB5*0101/DRA, Fla-2/Fla-X₅₁₋₆₅-HLA-DRB4*0101/DRA, Fla-2/Fla-X₂₆₉₋₂₈₃-HLA-DRB4*0101/DRA, Fla-2/Fla-X₄₋₁₈-HLA-DRB3*0202/DRA, Fla-2/Fla-X₂₆₁₋₂₇₅-HLA-DRB3*0202/DRA, Fla-2/Fla-X₂₇₁₋₂₈₅-HLA-DRB3*0202/DRA, YIDX₇₈₋₉₂-HLA-DRB3*0101/DRA, YIDX₇₈₋₉₂-HLA-DRB4*0101/DRA, YIDX₉₃₋₁₀₇-HLA-DRB3*0101/DRA, YIDX₉₈₋₁₁₂-HLA-DRB5*0101/DRA, YIDX₂₃₋₃₇-HLA-DRB5*0101/DRA, YIDX₇₈₋₉₂-HLA-DRB4*0101/DRA, YIDX₁₉₅₋₂₀₉-HLA-DRB4*0101/DRA, YIDX₂₂₋₃₆-HLA-DRB3*0202/DRA, YIDX₈₀₋₉₄-HLA-DRB3*0202/DRA, or YIDX₁₀₁₋₁₁₅-HLA-DRB3*0202/DRA;
i) COPD and/or emphysema and the pMHC complex is selected from the group of: elastin₈₉₋₁₀₃-HLA-DRB3*0101/DRA, elastin₆₉₈₋₇₁₂-HLA-DRB5*0101/DRA, elastin₈₋₂₂-HLA-DRB5*0101/DRA, elastin₉₄₋₁₀₈-HLA-DRB5*0101/DRA, elastin₁₃₋₂₇-HLA-DRB4*0101/DRA, elastin₆₉₅₋₇₀₉-HLA-DRB4*0101/DRA, elastin₅₆₃₋₅₇₇-HLA-DRB4*0101/DRA, elastin₅₅₈₋₅₇₂-HLA-DRB4*0101/DRA, elastin₆₉₈₋₇₁₂-HLA-DRB5*0101/DRA, elastin₅₆₆₋₅₈₀-HLA-DRB3*0202/DRA, or elastin₆₄₅₋₆₅₉-HLA-DRB3*0202/DRA;
j) psoriasis and the pMHC complex is selected from the group of: Cap18₆₄₋₇₈-HLA-DRB3*0101/DRA, Cap18₃₄₋₄₈-HLA-DRB3*0101/DRA, Cap18₄₇₋₆₁-HLA-DRB3*0101/DRA, Cap18₁₅₁₋₁₆₅-HLA-DRB4*0101/DRA, Cap18₁₄₉₋₁₆₃-HLA-DRB5*0101/DRA, Cap18₁₅₂₋₁₆₆-HLA-DRB5*0101/DRA, Cap18₁₃₁₋₁₄₅-HLA-DRB5*0101/DRA, Cap18₂₄₋₃₈-HLA-DRB3*0202/DRA, ADMTSL5₂₄₅₋₂₅₉-HLA-DRB3*0101/DRA, ADMTSL5₂₆₇₋₂₈₁-HLA-DRB3*0101/DRA, ADMTSL5₃₇₂₋₃₈₆-HLA-DRB3*0101/DRA, ADMTSL5₂₈₉₋₃₀₃-HLA-DRB4*0101/DRA, ADMTSL5₃₉₆₋₄₁₀-HLA-DRB4*0101/DRA, ADMTSL5₄₃₃₋₄₄₇-HLA-DRB4*0101/DRA, ADMTSL5₁₄₂₋₁₅₆-HLA-DRB5*0101/DRA, ADMTSL5₂₃₆₋₂₅₀-HLA-DRB5*0101/DRA, ADMTSL5₃₀₁₋₃₁₅-HLA-DRB5*0101/DRA, ADMTSL5₂₀₃₋₂₁₇-HLA-DRB3*0202/DRA, ADMTSL5₄₀₄₋₄₁₈-HLA-DRB3*0202/DRA, or ADMTSL5₄₃₃₋₄₄₇-HLA-DRB3*0202/DRA;
k) autoimmune hepatitis and the pMHC complex is selected from the group of: CYP2D6₁₉₃₋₂₀₇-HLA-DRB1*0301/DRA, CYP2D6₇₆₋₉₀-HLA-DRB1*0301/DRA, CYP2D6₂₉₃₋₃₀₇-HLA-DRB1*0301/DRA, CYP2D6₃₁₃₋₃₃₂-HLA-DRB1*0301/DRA, CYP2D6₃₉₃₋₄₁₂-HLA-DRB1*0301/DRA, CYP2D6₁₉₉₋₂₁₃-HLA-DRB1*0401/DRA, CYP2D6₄₅₀₋₄₆₄-HLA-DRB1*0401/DRA, CYP2D6₃₀₁₋₃₁₅-HLA-DRB1*0401/DRA, CYP2D6₄₅₂₋₄₆₆-HLA-DRB1*0701/DRA, CYP2D6₅₉₋₇₃-HLA-DRB1*0701/DRA, CYP2D6₁₃₀₋₁₄₄-HLA-DRB1*0701/DRA, CYP2D6₁₉₃₋₂₁₂-HLA-DRB1*0701/DRA, CYP2D6₃₀₅₋₃₂₄-HLA-DRB1*0701/DRA, CYP2D6₁₃₁₋₁₄₅-HLA-DRB3*0202/DRA, CYP2D6₂₁₆₋₂₃₀-HLA-DRB3*0202/DRA, CYP2D6₂₃₈₋₂₅₂-HLA-DRB3*0202/DRA, CYP2D6₁₉₉₋₂₁₃-HLA-DRB4*0101/DRA, CYP2D6₂₃₅₋₂₅₂-HLA-DRB4*0101/DRA, CYP2D6₂₉₃₋₃₀₇-HLA-DRB4*0101/DRA, CYP2D6₂₃₈₋₂₅₂-HLA-DRB5*0101/DRA, CYP2D6₃₈₁₋₃₉₅-HLA-DRB5*0101/DRA, CYP2D6₄₂₉₋₄₄₃-HLA-DRB5*0101/DRA, SLA₃₃₄₋₃₄₈-HLA-DRB1*0301/DRA, SLA₁₉₆₋₂₁₀-HLA-DRB1*0301/DRA, SLA₁₁₅₋₁₂₉-HLA-DRB1*0301/DRA, SLA₃₇₃₋₃₈₆-HLA-DRB1*0301/DRA, SLA₁₈₆₋₁₉₇-HLA-DRB1*0301/DRA, SLA₃₁₇₋₃₃₁-HLA-DRB1*0401/DRA, SLA₁₇₁₋₁₈₅-HLA-DRB1*0401/DRA, SLA₄₁₇₋₄₃₁-HLA-DRB1*0401/DRA, SLA₃₅₉₋₃₇₃-HLA-DRB1*0701/DRA, SLA₂₁₅₋₂₂₉-HLA-DRB1*0701/DRA, SLA₁₁₁₋₁₂₅-HLA-DRB1*0701/DRA, SLA₁₁₀₋₁₂₄-HLA-DRB3*0202/DRA, SLA₂₉₉₋₃₁₃-HLA-DRB3*0202/DRA, SLA₃₄₂₋₃₅₆-HLA-DRB3*0202/DRA, SLA₄₉₋₆₃-HLA-DRB4*0101/DRA, SLA₁₁₉₋₁₃₃-HLA-DRB4*0101/DRA, SLA₂₆₀₋₂₇₄-HLA-DRB4*0101/DRA, SLA₂₆₋₄₀-HLA-DRB5*0101/DRA, SLA₈₆₋₁₀₀-HLA-DRB5*0101/DRA, or SLA₃₃₁₋₃₄₅-HLA-DRB5*0101/DRA;
l) uveitis and the pMHC complex is selected from the group of: arrestin₁₉₉₋₂₁₃-HLA-DRB3*0101/DRA, arrestin₇₇₋₉₁-HLA-DRB3*0101/DRA, arrestin₂₅₀₋₂₆₄-HLA-DRB3*0101/DRA, arrestin₁₇₂₋₁₈₆-HLA-DRB4*0101/DRA, arrestin₃₅₄₋₃₆₈-HLA-DRB4*0101/DRA, arrestin₂₃₉₋₂₅₃-HLA-DRB4*0101/DRA, arrestin₁₀₂₋₁₁₆-HLA-DRB5*0101/DRA, arrestin₅₉₋₇₃-HLA-DRB5*0101, arrestin₂₈₀₋₂₉₄-HLA-DRB5*0101, arrestin₂₉₁₋₃₀₆-HLA-DRB1*0301/DRA, arrestin₁₉₅₋₂₀₉-HLA-DRB3*0202/DRA, arrestin₁₉₉₋₂₁₃-HLA-DRB3*0202/DRA, or arrestin₂₀₀₋₂₁₄-HLA-DRB3*0202/DRA;
m) Sjogren Syndrome and the pMHC complex is selected from the group of: RO60₁₂₇₋₁₄₁-HLA-DRB1*0301/DRA, RO60₅₂₃₋₅₃₇-HLA-DRB1*0301/DRA, RO60₂₄₃₋₂₅₇-HLA-DRB1*0301/DRA, RO60₄₈₄₋₄₉₈-HLA-DRB3*0101/DRA, RO60₃₄₇₋₃₆₁-HLA-DRB3*0101/DRA, RO60₃₆₉₋₃₈₃-HLA-DRB3*0101/DRA, RO60₄₂₆₋₄₄₀-HLA-DRB4*0101/DRA, RO60₂₆₇₋₂₈₁-HLA-DRB4*0101/DRA, RO60₁₇₈₋₁₉₂-HLA-DRB4*0101/DRA, RO60₃₅₈₋₃₇₂-HLA-DRB5*0101/DRA, RO60₃₅₈₋₃₇₂-HLA-DRB4*0101/DRA, RO60₂₂₁₋₂₃₅-HLA-DRB5*0101/DRA, RO60₂₂₁₋₂₃₅-HLA-DRB4*0101/DRA, RO60₃₁₈₋₃₃₂-HLA-DRB5*0101/DRA, RO60₃₁₈₋₃₃₂-HLA-DRB4*0101/DRA, RO60₄₀₇₋₄₂₁-HLA-DRB4*0101/DRA, RO60₄₀₇₋₄₂₁-HLA-DQA1*0501/HLA-DQB1*0201, RO60₄₅₉₋₄₇₃-HLA-DRB4*0101/DRA, RO60₄₅₉₋₄₇₃-HLA-DQA1*0501/HLA-DQB1*0201, RO60₃₁₈₋₃₃₂-HLA-DQA1*0501/HLA-DQB1*0201, RO60₅₁₋₆₅-HLA-DRB3*0202/DRA, RO60₃₁₂₋₃₂₆-HLA-DRB3*0202/DRA, RO60₃₄₇₋₃₆₁-HLA-DRB3*0202/DRA, LA₂₄₁₋₂₅₅-HLA-DRB1*0301/DRA, LA₁₀₁₋₁₁₅-HLA-DRB1*0301/DRA, LA₁₅₃₋₁₆₇-HLA-DRB1*0301/DRA, LA₁₇₈₋₁₉₂-HLA-DRB3*0101/DRA, LA₁₉₋₃₃-HLA-DRB3*0101/DRA, LA₃₇₋₅₁-HLA-DRB3*0101/DRA, LA₁₃₃₋₁₄₇-HLA-DRB4*0101/DRA, LA₅₀₋₆₄-HLA-DRB4*0101/DRA, LA₃₂₄₆-HLA-DRB4*0101/DRA, LA₁₅₃₋₁₆₇-HLA-DRB5*0101/DRA, LA₈₃₋₉₇-HLA-DRB5*0101/DRA, LA₁₃₆₋₁₅₀-HLA-DRB5*0101/DRA, LA₂₉₇₋₃₁₁-HLA-DQA1*0501/HLA-DQB1*0201, LA₅₉₋₇₃-HLA-DQA1*0501/HLA-DQB1*0201, LA₅₉₋₇₃-HLA-DRB4*0101/DRA, LA₁₅₁₋₁₆₅-HLA-DQA1*0501/HLA-DQB1*0201, LA₁₅₁₋₁₆₅-HLA-DRB4*0101/DRA, LA₂₉₇₋₃₁₁-HLA-DRB4*0101/DRA, LA₅₀₋₆₄-HLA-DRB3*0202/DRA, LA₈₆₋₁₀₀-HLA-DRB3*0202/DRA, or LA₁₅₄₋₁₆₈-HLA-DRB3*0202/DRA;
n) scleroderma and the pMHC complex is selected from the group of: TOP1₃₄₆₋₃₆₀-HLA-DRB3*0101/DRA, TOP1₄₂₀₋₄₃₄-HLA-DRB3*0101/DRA, TOP1₇₅₀₋₇₆₄-HLA-DRB3*0101/DRA, TOP1₄₁₉₋₄₃₃-HLA-DRB4*0101/DRA, TOP1₅₉₁₋₆₀₅-HLA-DRB4*0101/DRA, TOP1₆₉₅₋₇₀₉-HLA-DRB4*0101/DRA, TOP1₃₀₅₋₃₁₉-HLA-DRB5*0101/DRA, TOP1₃₄₆₋₃₆₀-HLA-DRB5*0101/DRA, TOP1₄₁₉₋₄₃₃-HLA-DRB5*0101/DRA, TOP1₄₂₀₋₄₃₄-HLA-DRB3*0202/DRA, TOP1₄₂₅₋₄₃₉-HLA-DRB3*0202/DRA, TOP1₆₁₄₋₆₂₈-HLA-DRB3*0202/DRA, CENP-C₂₉₇₋₃₁₁-HLA-DRB3*0101/DRA, CENP-C₈₅₇₋₈₇₁-HLA-DRB3*0101/DRA, CENP-C₈₈₇₋₉₀₁-HLA-DRB3*0101/DRA, CENP-C₂₁₂₋₂₂₆-HLA-DRB4*0101/DRA, CENP-C₆₄₃₋₆₅₇-HLA-DRB4*0101/DRA, CENP-C₈₃₂₋₈₄₆-HLA-DRB4*0101/DRA, CENP-C₁₆₇₋₁₈₁-HLA-DRB5*0101/DRA, CENP-C₂₄₆₋₂₆₀-HLA-DRB5*0101/DRA, CENP-C₈₄₆₋₈₆₀-HLA-DRB5*0101/DRA, CENP-C₁₄₉₋₁₆₃-HLA-DRB3*0202/DRA, CENP-C₈₃₃₋₈₄₇-HLA-DRB3*0202/DRA, or CENP-C₈₄₇₋₈₆₁-HLA-DRB3*0202/DRA;
o) anti-phospholipid syndrome and the pMHC complex is selected from the group of: APOH₂₃₅₋₂₄₉-HLA-DRB3*0101/DRA, APOH₃₀₆₋₃₂₀-HLA-DRB3*0101/DRA, APOH₂₃₇₋₂₅₁-HLA-DRB3*0101/DRA, APOH₂₉₅₋₃₀₉-HLA-DRB3*0101/DRA, APOH₂₈₋₄₂-HLA-DRB4*0101/DRA, APOH₁₇₃₋₁₈₇-HLA-DRB4*0101/DRA, APOH₂₆₄₋₂₇₈-HLA-DRB4*0101/DRA, APOH₂₉₅₋₃₀₉-HLA-DRB4*0101/DRA, APOH₄₉₋₆₃-HLA-DRB5*0101/DRA, APOH₂₆₉₋₂₈₃-HLA-DRB5*0101/DRA, APOH₂₉₅₋₃₀₉-HLA-DRB5*0101/DRA, APOH₃₂₁₋₃₅₅-HLA-DRB3*0202/DRA, APOH₃₂₂₋₃₃₆-HLA-DRB3*0202/DRA, or APOH₃₂₄₋₃₃₈-HLA-DRB3*0202/DRA;
p) ANCA-associated vasculitis and the pMHC complex is selected from the group of: MPO₅₀₆₋₅₂₀-HLA-DRB3*0101/DRA, MPO₃₀₂₋₃₁₆-HLA-DRB3*0101/DRA, MPO₇₋₂₁-HLA-DRB3*0101/DRA, MPO₆₈₉₋₇₀₃-HLA-DRB4*0101/DRA, MPO₂₄₈₋₂₆₂-HLA-DRB4*0101/DRA, MPO₄₄₄₋₄₅₈-HLA-DRB4*0101/DRA, MPO₅₁₃₋₅₂₇-HLA-DRB5*0101/DRA, MPO₉₇₋₁₁₁-HLA-DRB5*0101/DRA, MPO₆₁₆₋₆₃₀-HLA-DRB5*0101/DRA, MPO₄₆₂₋₄₇₆-HLA-DRB3*0202/DRA, MPO₆₁₇₋₆₃₁-HLA-DRB3*0202/DRA, MPO₇₁₄₋₇₂₈-HLA-DRB3*0202/DRA, PRTN3₄₄₋₅₈-HLA-DRB3*0101/DRA, PRTN3₂₃₄₋₂₄₈-HLA-DRB3*0101/DRA, PRTN3₅₉₋₇₃-HLA DRB3*0101/DRA, PRTN3₅₉₋₇₃-HLA-DRB5*0101/DRA, PRTN3₁₁₇₋₁₃₁-HLA-DRB4*0101/DRA, PRTN3₁₆₄₋₁₇₈-HLA-DRB4*0101/DRA, PRTN3₇₁₋₅₅-HLA-DRB4*0101/DRA, PRTN3₂₄₁₋₂₅₅-HLA-DRB5*0101/DRA, PRTN3₁₈₃₋₁₉₇-HLA-DRB5*0101/DRA, PRTN3₆₂₋₇₆-HLA-DRB3*0202/DRA, PRTN3₁₁₈₋₁₃₂-HLA-DRB3*0202/DRA, or PRTN3₂₃₉₋₂₅₃-HLA-DRB3*0202/DRA; or
q) Stiff Man Syndrome and the pMHC complex is selected from the group of: GAD₂₁₂₋₂₂₆-HLA-DRB1*0801/DRA, GAD₅₅₅₋₅₆₉-HLA-DRB1*0801/DRA, or GAD₂₉₇₋₃₁₁-HLA-DRB1*0301/DRA.

109. The complex of any of claims 82-85 and 88-108, wherein the pMHC complex is for the treatment of:
a) type I diabetes and the pMHC complex is selected from the group of: PPI_{76-90(K88S)}-HLA-DRB1*0401/DRA, IGRP₁₃₋₂₅-HLA-DRB1*0301/DRA, GAD₅₅₅₋₅₆₇ HLA-DRB1*0401/DRA, GAD_{555-567(557I)}-HLA-DRB1*0401/DRA, IGRP₂₃₋₃₅-HLA-DRB1*0401/DRA, or PPI₇₆₉₀-HLA-DRB1*0401/DRA;
b) multiple sclerosis and the pMHC complex is selected from the group of: MBP₈₆₋₉₈-HLA-DRB1*1501/DRA, MBP₈₉₋₁₀₁-HLA-DRB5*0101/DRA, MOG₃₈₋₅₂-HLA-DRB4*0101/DRA, MOG_{97-109(E107S)}-HLA-DRB1*0401/DRA, MOG₂₀₃₋₂₁₇-HLA-DRB3*0101/DRA, PLP₅₄₋₆₈-HLA-DRB3*0101/DRA, PLP₉₄₋₁₀₈-HLA-DRB1*0301/DRA, PLP₂₅₀₋₂₆₄-HLA-DRB4*0101/DRA, MPB₁₃₋₃₂-HLA-DRB5*0101/DRA, MPB₈₃₋₉₉-HLA-DRB5*0101/DRA, MPB₁₁₁₋₁₂₉-HLA-DRB5*0101/DRA, MPB₁₄₆₋₁₇₀-HLA-DRB5*0101/DRA, MOG₂₂₃₋₂₃₇-HLA-DRB3*0202/DRA, MOG₆₋₂₀-HLA-DRB5*0101/DRA, PLP₈₈₋₁₀₂-HLA-DRB3*0202/DRA, or PLP₁₃₉₋₁₅₄-HLA-DRB5*0101/DRA;
c) Celiac Disease and the pMHC complex is selected from the group of: aGlia₅₇₋₆₈-HLA-DQA1*0501/HLA-DQB1*0201, aGlia₆₂₋₇₂-HLA-DQA1*0501/HLA-DQB1*0201, or aGlia₂₁₇₋₂₂₉-HLA-DQA1*0501/HLA-DQB1*0302;
d) primary biliary cirrhosis and the pMHC complex is selected from the group of: PDC-E2₁₂₂₋₁₃₅-HLA-DRB4*0101/DRA, PDC-E2₂₄₉₋₂₆₂-HLA-DRB4*0101/DRA, PDC-E2₂₄₉₋₂₆₃-HLA-DRB1*0801/DRA, PDC-E2₆₂₉₋₆₄₃-HLA-DRB1*0801/DRA, PDC-E2₇₂₋₈₆-HLA-DRB3*0202/DRA, PDC-E2₃₅₃₋₃₆₇-HLA-DRB3*0202/DRA, PDC-E2₄₂₂₋₄₃₆-HLA-DRB3*0202/DRA, PDC-E2₆₂₉₋₆₄₃-HLA-DRB4*0101/DRA, PDC-E2₈₀₋₉₄-HLA-DRB5*0101/DRA, PDC-E2₃₅₃₋₃₆₇-HLA-DRB5*0101/DRA, or PDC-E2₅₃₅₋₅₄₉-HLA-DRB5*0101/DRA;
e) pemphigus folliaceus and/or pemphigus vulgaris and the pMHC complex is selected from the group of: DG1₂₁₆₋₂₂₉-HLA-DRB1*0101/DRA, DG3₉₇₋₁₁₁-HLA-DRB1*0402/DRA, DG3₂₅₁₋₂₆₅-HLA-DRB1*0401/DRA, DG3₄₄₁₋₄₅₅-HLA-DRB1*0402/DRA, DG3₃₅₁₋₃₆₅-HLA-DRB3*0202/DRA, DG3₄₅₃₋₄₆₇-HLA-DRB3*0202/DRA, DG3₅₄₀₋₅₅₄-HLA-DRB3*0202/DRA, DG3₂₈₀₋₂₉₄-HLA-DRB4*0101/DRA, DG3₃₂₆₋₃₄₀-HLA-DRB4*0101/DRA, DG3₃₆₇₋₃₈₁-HLA-DRB4*0101/DRA, DG3₁₃₋₂₇-HLA-DRB5*0101/DRA, DG3₃₂₃₋₃₃₇-HLA-DRB5*0101/DRA, DG3₄₃₈₋₄₅₂-HLA-DRB5*0101/DRA, DG1₄₈₋₆₂-HLA-DRB3*0202/DRA, DG1₂₀₆₋₂₂₂-HLA-DRB3*0202/DRA, DG1₃₆₃₋₃₇₇-HLA-DRB3*0202/DRA, DG1₃₋₁₇-HLA-DRB4*0101/DRA, DG1₁₉₂₋₂₀₆-HLA-DRB4*0101/DRA, DG1₃₂₆₋₃₄₀-HLA-DRB4*0101/DRA, DG1₁₋₁₅-HLA-DRB5*0101/DRA, DG1₃₅₋₄₉-HLA-DRB5*0101/DRA, or DG1₃₂₅₋₃₃₉-HLA-DRB5*0101/DRA;
f) neuromyelitis optica spectrum disorder and the pMHC complex is selected from the group of: AQP4₂₈₄₋₂₉₈-HLA-DRB1*0301/DRA, AQP4₆₃₋₇₆-HLA-DRB1*0301/DRA, AQP4₁₂₉₋₁₄₃-HLA-DRB1*0401/DRA, or AQP4₃₉₋₅₃-HLA-DRB1*1501/DRA;
g) allergic asthma and the pMHC complex is selected from the group of: DERP-1₁₆₋₃₀-HLA-DRB1*0101/DRA, DERP-1₁₆₋₃₀-HLA-DRB1*1501/DRA, DERP1₁₇₁₋₁₈₅-HLA-DRB1*1501/DRA, DERP-1₁₁₀₋₁₂₄-HLA-DPB1*0401/DRA, DERP-2₂₆₋₄₀-HLA-DRB1*0101/DRA; DERP-2₂₆₋₄₀-HLA-DRB1*1501/DRA, or DERP-2₁₀₇₋₁₂₁-HLA-DRB1*0301/DRA;
h) inflammatory bowel disease and the pMHC complex is selected from the group of: bacteroides integrase antigen₁₋₁₅-HLA-DRB5*0101/DRA, bacteroides integrase antigen₁₈₃₋₁₉₇-HLA-DRB3*0101/DRA, bacteroides integrase antigen₇₀₋₈₄-HLA-DRB4*0101/DRA, bacteroides integrase antigen₄₋₁₈-HLA-DRB3*0202/DRA, bacteroides integrase antigen₁₇₁₋₁₈₅-HLA-DRB3*0202/DRA, bacteroides integrase antigen₂₅₆₋₂₇₀-HLA-DRB3*0202/DRA, Fla-2/Fla-X₃₆₆₋₃₈₀-HLA-DRB3*0101/DRA, Fla-2/Fla-X₂₆₁₋₂₇₅-HLA-DRB5*0101/DRA, Fla-2/Fla-X₅₁₋₆₅-HLA-DRB4*0101/DRA, Fla-2/Fla-X₄₋₁₈-HLA-DRB3*0202/DRA, Fla-2/Fla-X₂₆₁₋₂₇₅-HLA-DRB3*0202/DRA, Fla-2/Fla-X₂₇₁₋₂₈₅-HLA-DRB3*0202/DRA, YIDX₇₈₋₉₂-HLA-DRB3*0101/DRA, YIDX₇₈₋₉₂-HLA-DRB4*0101/DRA, YIDX₉₈₋₁₁₂-HLA-DRB5*0101/DRA, YIDX₂₂₋₃₆-HLA-DRB3*0202/DRA, YIDX₈₀₋₉₄-HLA-DRB3*0202/DRA, or YIDX₁₀₋₁₁₅-HLA-DRB3*0202/DRA;
i) emphysema and the pMHC complex is selected from the group of: elastin₈₉₋₁₀₃-HLA-DRB3*0101/DRA, elastin₆₉₈₋₇₁₂-HLA-DRB5*0101/DRA, elastin₅₅₈₋₅₇₂-HLA-DRB4*0101/DRA, elastin₅₆₆₋₅₈₀-HLA-DRB3*0202/DRA, or elastin₅₄₅₋₆₅₉-HLA-DRB3*0202/DRA;
j) psoriasis and the pMHC complex is selected from the group of: Cap18₆₄₋₇₈-HLA-DRB3*0101/DRA, Cap18₃₄₋₄₈-HLA-DRB3*0101/DRA, Cap18₄₇₋₆₁-HLA-DRB3*0101/DRA, Cap18₁₅₁₋₁₆₅-HLA-DRB4*0101/DRA, Cap18₁₄₉₋₁₆₃-HLA-DRB5*0101/DRA, Cap18₁₅₂₋₁₆₆-HLA-DRB5*0101/DRA, Cap18₁₃₁₋₁₄₅-HLA-DRB5*0101/DRA, Cap₁₈₂₄₋₃₈-HLA-DRB3*0202/DRA, ADMTSL5₂₄₅₋₂₅₉-HLA-DRB3*0101/DRA, ADMTSL5₂₆₇₋₂₈₁-HLA-DRB3*0101/DRA, ADMTSL5₃₇₂₋₃₈₆-HLA-DRB3*0101/DRA, ADMTSL5₂₈₉₋₃₀₃-HLA-DRB4*0101/DRA, ADMTSL5₃₉₆₋₄₁₀-HLA-DRB4*0101/DRA, ADMTSL5₄₃₃₋₄₄₇-HLA-DRB4*0101/DRA, ADMTSL5₁₄₂₋₁₅₆-HLA-DRB5*0101/DRA, ADMTSL5₂₃₆₋₂₅₀-HLA-DRB5*0101/DRA, ADMTSL5₃₀₁₋₃₁₅-HLA-DRB5*0101/DRA, ADMTSL5₂₀₃₋₂₁₇-HLA-DRB3*0202/DRA, ADMTSL5₄₀₄₋₄₁₈-HLA-DRB3*0202/DRA, or ADMTSL5₄₃₃₋₄₄₇-HLA-DRB3*0202/DRA;
k) autoimmune hepatitis and the pMHC complex is selected from the group of: CYP2D6₁₉₃₋₂₀₇-HLA-DRB1*0301/DRA, CYP2D6₇₆₋₉₀-HLA-DRB1*0301/DRA, CYP2D6₂₉₃₋₃₀₇-HLA-DRB1*0301/DRA, CYP2D6₃₁₃₋₃₃₂-HLA-DRB1*0301/DRA, CYP2D6₃₉₃₋₄₁₂-HLA-DRB1*0301/DRA, CYP2D6₁₉₉₋₂₁₃-HLA-DRB1*0401/DRA, CYP2D6₄₅₀₋₄₆₄-HLA-DRB1*0401/DRA, CYP2D6₃₀₁₋₃₁₅-HLA-DRB1*0401/DRA, CYP2D6₄₅₂₋₄₆₆-HLA-DRB1*0701/DRA, CYP2D6₅₉₋₇₃-HLA-DRB1*0701/DRA, CYP2D6₁₃₀₋₁₄₄-HLA-DRB1*0701/DRA, CYP2D6₁₉₃₋₂₁₂-HLA-DRB1*0701/DRA, CYP2D6₃₀₅₋₃₂₄-HLA-DRB1*0701/DRA, CYP2D6₁₃₁₋₁₄₅-HLA-DRB3*0202/DRA, CYP2D6₂₁₆₋₂₃₀-HLA-DRB3*0202/DRA, CYP2D6₂₃₈₋₂₅₂-HLA-DRB3*0202/DRA, CYP2D6₁₉₉₋₂₁₃-HLA-DRB4*0101/DRA, CYP2D6₂₃₅₋₂₅₂-HLA-DRB4*0101/DRA, CYP2D6₂₉₃₋₃₀₇-HLA-DRB4*0101/DRA, CYP2D6₂₃₈₋₂₅₂-HLA-DRB5*0101/DRA, CYP2D6₃₈₁₋₃₉₅-HLA-DRB5*0101/DRA, CYP2D6₄₂₉₋₄₄₃-HLA-DRB5*0101/DRA, SLA₃₃₄₋₃₄₈-HLA-DRB1*0301/DRA, SLA₁₉₆₋₂₁₀-HLA-DRB1*0301/DRA, SLA₁₁₅₋₁₂₉-HLA-DRB1*0301/DRA, SLA₃₇₃₋₃₈₆-HLA-DRB1*0301/DRA, SLA₁₈₆₋₁₉₇-HLA-DRB1*0301/DRA, SLA₃₁₇₋₃₃₁-HLA-DRB1*0401/DRA, SLA₁₇₁₋₁₈₅-HLA-DRB1*0401/DRA, SLA₄₁₇₋₄₃₁-HLA-DRB1*0401/DRA, SLA₃₅₉₋₃₇₃-HLA-DRB1*0701/DRA, SLA₂₁₅₋₂₂₉-HLA-DRB1*0701/DRA, SLA₁₁₁₋₁₂₅-HLA-DRB1*0701/DRA, SLA₁₁₀₋₁₂₄-HLA-DRB3*0202/DRA, SLA₂₉₉₋₃₁₃-HLA-DRB3*0202/DRA, SLA₃₄₂₋₃₅₆-HLA-DRB3*0202/DRA, SLA₄₉₋₆₃-HLA-DRB4*0101/DRA, SLA₁₁₉₋₁₃₃-HLA-DRB4*0101/DRA, SLA₂₆₀₋₂₇₄-HLA-DRB4*0101/DRA, SLA₂₆₋₄₀-HLA-DRB5*0101/DRA, SLA₈₆₋₁₀₀-HLA-DRB5*0101/DRA, or SLA₃₃₁₋₃₄₅-HLA-DRB5*0101/DRA;
l) uveitis and the pMHC complex is selected from the group of: arrestin₁₉₉₋₂₁₃-HLA-DRB3*0101/DRA, arrestin₇₇₋₉₁-HLA-DRB3*0101/DRA, arrestin₂₅₀₋₂₆₄-HLA-DRB3*0101/DRA, arrestin₁₇₂₋₁₈₆-HLA-DRB4*0101/DRA, arrestin₃₅₄₋₃₆₈-HLA-DRB4*0101/DRA, arrestin₂₃₉₋₂₅₃-HLA-DRB4*0101/DRA, arrestin₁₀₂₋₁₁₆-HLA-DRB5*0101/DRA, arrestin₅₉₋₇₃-HLA-DRB5*0101, arrestin₂₈₀₋₂₉₄-HLA-DRB5*0101, arrestin₂₉₁₋₃₀₆-HLA-DRB1*0301/DRA, arrestin₁₉₅₋₂₀₉-HLA-DRB3*0202/DRA, arrestin₁₉₉₋₂₁₃-HLA-DRB3*0202/DRA, or arrestin₂₀₀₋₂₁₄-HLA-DRB3*0202/DRA;
m) Sjogren Syndrome and the pMHC complex is selected from the group of: RO60₁₂₇₋₁₄₁-HLA-DRB1*0301/DRA, RO60₅₂₃₋₅₃₇-HLA-DRB1*0301/DRA, RO60₂₄₃₋₂₅₇-HLA-DRB1*0301/DRA, RO60₄₈₄₋₄₉₈-HLA-DRB3*0101/DRA, RO60₃₄₇₋₃₆₁-HLA-DRB3*0101/DRA, RO60₃₆₉₋₃₈₃-HLA-DRB3*0101/DRA, RO60₄₂₆₋₄₄₀-HLA-DRB4*0101/DRA, RO60₂₆₇₋₂₈₁-HLA-DRB4*0101/DRA, RO60₁₇₈₋₁₉₂-HLA-DRB4*0101/DRA, RO60₃₅₈₋₃₇₂-HLA-DRB5*0101/DRA, RO60₂₂₁₋₂₃₅-HLA-DRB5*0101/DRA, RO60₃₁₈₋₃₃₂-HLA-DRB5*0101/DRA, RO60₅₁₋₆₅-HLA-DRB3*0202/DRA, RO60₃₁₂₋₃₂₆-HLA-DRB3*0202/DRA, RO60₃₄₇₋₃₆₁-HLA-DRB3*0202/DRA, LA₂₄₁₋₂₅₅-HLA-DRB1*0301/DRA, LA₁₀₁₋₁₁₅-HLA-DRB1*0301/DRA, LA₁₅₃₋₁₆₇-HLA-DRB1*0301/DRA, LA₁₇₈₋₁₉₂-HLA-DRB3*0101/DRA, LA₁₉₋₃₃-HLA-DRB3*0101/DRA, LA₃₇₋₅₁-HLA-DRB3*0101/DRA, LA₁₃₃₋₁₄₇-HLA-DRB4*0101/DRA, LA₅₀₋₆₄-HLA-DRB4*0101/DRA, LA₃₂₋₄₆-HLA-DRB4*0101/DRA, LA₁₅₃₋₁₆₇-HLA-DRB5*0101/DRA, LA₈₃₋₉₇-HLA-DRB5*0101/DRA, LA₁₃₆₋₁₅₀-HLA-DRB5*0101/DRA, LA₅₀₋₆₄-HLA-DRB3*0202/DRA, LA₈₆₋₁₀₀-HLA-DRB3*0202/DRA, or LA₁₅₄₋₁₆₈-HLA-DRB3*0202/DRA;
n) scleroderma and the pMHC complex is selected from the group of: TOP1₃₄₆₋₃₆₀-HLA-DRB3*0101/DRA, TOP1₄₂₀₋₄₃₄-HLA-DRB3*0101/DRA, TOP1₇₅₀₋₇₆₄-HLA-DRB3*0101/DRA, TOP1₄₁₉₋₄₃₃-HLA-DRB4*0101/DRA, TOP1₅₉₁₋₆₀₅-HLA-DRB4*0101/DRA, TOP1₆₉₅₋₇₀₉-HLA-DRB4*0101/DRA, TOP1₃₀₅₋₃₁₉-HLA-DRB5*0101/DRA, TOP1₃₄₆₋₃₆₀-HLA-DRB5*0101/DRA, TOP1₄₁₉₋₄₃₃-HLA-DRB5*0101/DRA, TOP1₄₂₀₋₄₃₄-HLA-DRB3*0202/DRA, TOP1₄₂₅₋₄₃₉-HLA-DRB3*0202/DRA, TOP1₆₁₄₋₆₂₈-HLA-DRB3*0202/DRA, CENP-C₂₉₇₋₃₁₁-HLA-DRB3*0101/DRA, CENP-C₈₅₇₋₈₇₁-HLA-DRB3*0101, CENP-C₈₈₇₋₉₀₁-HLA-DRB3*0101, CENP-C₂₁₂₋₂₂₆-HLA-DRB4*0101/DRA, CENP-C₆₄₃₋₆₅₇-HLA-DRB4*0101/DRA, CENP-C₈₃₂₋₈₄₆-HLA-DRB4*0101/DRA, CENP-C₁₆₇₋₁₈₁-HLA-DRB5*0101/DRA, CENP-C₂₄₆₋₂₆₀-HLA-DRB5*0101/DRA, CENP-C₈₄₆₋₈₆₀-HLA-DRB5*0101/DRA, CENP-C₁₄₉₋₁₆₃-HLA-DRB3*0202/DRA, CENP-C₈₃₃₋₈₄₇-HLA-DRB3*0202/DRA, or CENP-C₈₄₇₋₈₆₁-HLA-DRB3*0202/DRA;
o) anti-phospholipid syndrome and the pMHC complex is selected from the group of: APOH₂₃₅₋₂₄₉-HLA-DRB3*0101/DRA, APOH₃₀₆₋₃₂₀-HLA-DRB3*0101/DRA, APOH₂₃₇₋₂₅₁-HLA-DRB3*0101/DRA, APOH₂₉₅₋₃₀₉-HLA-DRB3*0101/DRA, APOH₂₈₋₄₂-HLA-DRB4*0101/DRA, APOH₁₇₃₋₁₈₇-HLA-DRB4*0101/DRA, APOH₂₆₄₋₂₇₈-HLA-DRB4*0101/DRA, APOH₂₉₅₋₃₀₉-HLA-DRB4*0101/DRA, APOH₄₉₋₆₃-HLA-DRB5*0101/DRA, APOH₂₆₉₋₂₈₃-HLA-DRB5*0101/DRA, APOH₂₉₅₋₃₀₉-HLA-DRB5*0101/DRA, APOH₃₂₁₋₃₅₅-HLA-DRB3*0202/DRA, APOH₃₂₂₋₃₃₆-HLA-DRB3*0202/DRA, or APOH₃₂₄₋₃₃₈-HLA-DRB3*0202/DRA;
p) ANCA-associated vasculitis and the pMHC complex is selected from the group of: MPO₅₀₆₋₅₂₀-HLA-DRB3*0101/DRA, MPO₃₀₂₋₃₁₆-HLA-DRB3*0101/DRA, MPO₇₋₂₁-HLA-DRB3*0101/DRA, MPO₆₈₉₋₇₀₃-HLA-DRB4*0101/DRA, MPO₂₄₈₋₂₆₂-HLA-DRB4*0101/DRA, MPO₄₄₄₋₄₅₈-HLA-DRB4*0101/DRA, MPO₅₁₃₋₅₂₇-HLA-DRB5*0101/DRA, MPO₉₇₋₁₁₁-HLA-DRB5*0101/DRA, MPO₆₁₆₋₆₃₀-HLA-DRB5*0101/DRA, MPO₄₆₂₋₄₇₆-HLA-DRB3*0202/DRA, MPO₆₁₇₋₆₃₁-HLA-DRB3*0202/DRA, MPO₇₁₄₋₇₂₈-HLA-DRB3*0202/DRA, PRTN3₄₄₋₅₈-HLA-DRB3*0101/DRA, PRTN3₂₃₄₋₂₄₈-HLA-DRB3*0101/DRA, PRTN3₅₉₋₇₃-HLA DRB3*0101/DRA, PRTN3₅₉₋₇₃-HLA-DRB5*0101/DRA, PRTN3₁₁₇₋₁₃₁-HLA-DRB4*0101/DRA, PRTN3₁₆₄₋₁₇₈-HLA-DRB4*0101/DRA, PRTN3₇₁₋₈₅-HLA-DRB4*0101/DRA, PRTN3₂₄₁₋₂₅₅-HLA-DRB5*0101/DRA, PRTN3₁₈₃₋₁₉₇-HLA-DRB5*0101/DRA, PRTN3₆₂₋₇₆-HLA-DRB3*0202/DRA, PRTN3₁₁₈₋₁₃₂-HLA-DRB3*0202/DRA, or PRTN3₂₃₉₋₂₅₃-HLA-DRB3*0202/DRA; or
q) Stiff Man Syndrome and the pMHC complex is selected from the group of: GAD₂₁₂₋₂₂₆-HLA-DRB1*0801/DRA, GAD₅₅₅₋₅₆₉-HLA-DRB1*0801/DRA, or GAD₂₉₇₋₃₁₁-HLA-DRB1*0301/DRA

110. A composition comprising a plurality of complexes of any of claims 82-109.

111. The composition of claim 110, wherein one or more of: the diameters of each of the nanoparticle cores are the same or different from each other; and/or the valency of the pMHC complexes on each nanoparaticle core are the same or different from each other; and/or the density of the pMHC complexes on each nanoparticle core are the same or different from each other; and/or the density of the co-stimulatory molecules on each nanoparticle core are the same or different from each other; and/or the density of the cytokines on each nanoparticle core are the same or different from each other.

112. The composition of claim 110 or 111, further comprising further comprising one or more of a nanoparticle core coupled to one or more cytokines and/or co-stimulatory molecules,
wherein the nanoparticle core has a diameter selected from the group of: from about 1 nm to about 100 nm; from about 1 nm to about 50 nm; from about 1 nm to about 25 nm; from about 5 nm to about 100 nm; from about 5 nm to about 50 nm; or from about 5 nm to about 25 nm; and
optionally wherein the nanoparticle core further comprises an outer layer on the nanoparticle core.

113. A composition comprising a carrier and one or more of a complex of any of claims 82-109 and/or the composition of claims 110-112.

114. The composition of claim 113, wherein the carrier is a pharmaceutically acceptable carrier.

115. A method for differentiating an activated T cell or a memory T cell into a IL-10 producing T_{R}1 cell optionally expressing a marker CD49b and/or Lag3 and/or differentiating a B cell into a regulatory B cell, comprising contacting the T cells with an effective amount of the complex of any of claims 82-109 or the composition of claims 110-114, thereby differentiating the population of T cells, wherein the pMHC density of the nanoparticle is from about 0.4 pMHC/100 nm² to about 12 pMHC/100 nm².

116. The method of claim 115, wherein the contacting is in vitro or in vivo.

117. A method for differentiating an activated T cell or a memory T cell into a IL-10 producing T_{R}1 cell optionally expressing a marker CD49b and/or Lag3 and/or differentiating a B cell into a regulatory B cell in a subject in need thereof, comprising administering an effective amount of the complex of any of claims 82-109 or the composition of claims 110-114, wherein the pMHC density of the nanoparticle is from about 0.4 pMHC/100 nm² to about 12 pMHC/100 nm².

118. A dosage unit for the treatment of immune inflammation, an autoimmune disease, cancer, or an allergic disease comprising an effective amount of the complex comprising a disease-relevant pMHCmprising of any of the preceding claims and a pharmaceutically acceptable carrier.

119. A method for making, preparing, or obtaining the complex of any of claims 1-28, 37-70, and 82-109, comprising coating or complexing pMHC complexes onto the nanoparticle core, the nanoparticle dendrimer core or the polymeric micelle core, wherein the nanoparticle core, the nanoparticle dendrimer core or the polymeric micelle core optionally comprises an outer layer.

120. A method for promoting the formation of, expansion, and recruitment of a population of IL-10 producing T_{R}1 cell optionally expressing a marker CD49b and/or LAG3 in an antigen specific manner in a subject, comprising administering to the subject an effective amount of the complex of any of claims 1-28, 37-70, and 82-109.

121. A method for preventing or treating a disease or condition in a subject in need thereof, comprising administering to the subject in need thereof an effective amount of the complex of any of claims 1-28, 37-70, and 82-109 or the composition of any of claims 29-33, 71-75, and 110-114, thereby preventing or treating the disease, with the proviso that the disease-relevant antigen of the pMHC complex is relevant to the disease prevented or treated in the subject.

122. The method of claim 121, wherein the disease or condition is selected from the group of pre-diabetes, multiple sclerosis, allergic asthma, primary biliary cirrhosis, cirrhosis, Neuromyelitis optica spectrum disorder (Devic's disease, NMO), autoimmune encephalitis, autoantibody-mediated neurological syndromes, a Stiff Man syndrome, paraneoplastic disease, other diseases of the central and peripheral nervous systems, Pemphigus vulgaris, inflammatory bowel disease, Crohn's disease, Ulcerative Colitis, arthritis, Rheumatoid Arthritis, systemic lupus erythematosus (SLE), Celiac Disease, psoriasis, Alopecia Areata, Acquired Thrombocytopenic Purpura, autoimmune cardiomyopathy, idiopathic dilated cardiomyopathy (IDCM), Myasthyenia Gravis, Uveitis, Ankylosing Spondylitis, Grave's Disease, Hashimoto's thyroiditis, Immune Mediated Myopathies, anti-phospholipid syndrome (ANCA+), atherosclerosis, scleroderma, autoimmune hepatitis, dermatomyositis, chronic obstructive pulmonary disease, a spinal cord injury, traumatic injury, tobacco-induced lung destruction, Chronic Obstructive Pulmonary Disease (COPD), lung emphysema, sclerosing cholangitis, peripheral neuropathy, narcolepsy, Goodpasture Syndrome, Kawasaki's Disease, autoimmune uveitis, colitis, emphysema, pemphigus, pemphigus folliaceus, Sjogren's Syndrome, ANCA-associated vasculitis, primary sclerosing cholangitis-relevant antigen, adipose tissue inflammation/diabetes type II, or obesity associated adipose tissue inflammation/insulin resistance.

123. The method of any of claims 34-36, 76-81, and 115-117, wherein the subject is a mammal or a human patient.

124. A method to detect a population of T_{R}1 cells and/or effector T cells in an antigen specific manner in a subject that has received the complex of any one of claims 1-28, 37-70, and 82-109 or the composition of any of claims 29-33, 71-75, and 110-114, the method comprising contacting a sample suspected of comprising the T_{R}1 cells with an effective amount of labeled pMHC complex to form a multimer complex, and detecting any multimer complex, thereby detecting the population of T_{R}1 cells.

125. The method of claim 124, further comprising staining any T cell population using a labeled multimer complex.

126. The method of claim 124 or 125, wherein the step of detecting the population of T_{R}1 cells comprises flow cytometry to detect any multimer complex.

127. The method of any of claims 124-126, further comprising administering the complex of any one of claims 1-28, 37-70, and 82-109 or the composition of any of claims 29-33, 71-75, and 110-114 to the subject.

128. A method to detect a population of T_{R}1 cells and/or effector T cells in an antigen specific manner in a subject that has received the complex of any one of claims 1-28, 37-70, and 82-109 or the composition of any of claims 29-33, 71-75, and 110-114, the method comprising any one of the following assays: cytokine ELISPOT assay, a multimer-guided epitope analysis, or a multimer-pull-down assay.

129. The method of claim 128, further comprising administering the complex of any one of claims 1-28, 37-70, and 82-109 or the composition of any of claims 29-33, 71-75, and 110-114 to the subject.

130. A method to monitor the expansion of a population of antigen-specific T_{R}1 and/or effector T cells in a subject, the method comprising:
a) administering to a subject an effective amount of the complex of any of claims 1-28, 37-70, and 82-109 or the composition of any of claims 29-33, 71-75, and 110-114, wherein the disease-relevant antigen of the pMHC complex is selected to expand the antigen-specific T regulatory and/or effector T cells;
b) isolating a suitable sample from the subject suspected of containing the population;
c) contacting the sample with an effective amount of labeled pMHC complex to form a multimer complex, and detecting any multimer complex; and
d) quantifying the number of antigen-specific T_{R}1 and/or effector T cells in the population.

131. The method of claim 130, further comprising staining any multimer complex.

132. The method of claim 130 or 131, wherein the step of quantifying the number of antigen-specific T_{R}1 and/or effector T cells comprises flow cytometry.

133. The method of claim 132, wherein the step of quantifying the number of antigen-specific T_{R}1 and/or effector T cells comprises ELISA.

134. The method of any of claims 130-133, further comprising administering the complex of any one of claims 1-28, 37-70, and 82-109 or the composition of any of claims 29-33, 71-75, and 110-114.

135. A kit comprising the complex of any of claims 1-28, 37-70, and 82-109 or the composition of any of claims 29-33, 71-75, and 110-114.

136. The kit of claim 135 further comprising instructions for use in the methods of claims any of claims 34-36, 76-81, and 115-117.


SEQUENCE LISTING (504 sequences)

SEQ ID NO: 1 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 355 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 3 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 4 - protein, 470 aa, Homo sapiens
MSHHPSGLRAGFSSTSYRRTFGPPPSLSPGAFSYSSSSRFSSSRLLGSASPSSSVRLGSFRSPRAGAGALLRLPSERLDFSMAEALNQEFLATRSNEKQELQELNDRFANFIEKVRFLEQQNAALRGELSQARGQEPARADQLCQQELRELRRELELLGRERDRVQVERDGLAEDLAALKQRLEEETRKREDAEHNLVLFRKDVDDATLSRLELERKIESLMDEIEFLKKLHEEELRDLQVSVESQQVQQVEVEATVKPELTAALRDIRAQYESIAAKNLQEAEEWYKSKYADLSDAANRNHEALRQAKQEMNESRRQIQSLTCEVDGLRGTNEALLRQLRELEEQFALEAGGYQAGAARLEEELRQLKEEMARHLREYQELLNVKMALDIEIATYRKLLEGEESRISVPVHSFASLNIKTTVPEVEPPQDSHSRKTVLIKTIETRNGEVVTESQKEQRSELDKSSAHSY

SEQ ID NO: 5 - protein, 288 aa, Homo sapiens
MKTFLILALLAIVATTATTAVRVPVPQLQPQNPSQQQPQEQVPLVQQQQFLGQQQPFPPQQPYPQPQPFPSQQPYLQLQPFPQPQLPYSQPQPFRPQQPYPQPQPQYSQPQQPISQQQQQQQQQQQQQQQQQQQQILQQILQQQLIPCMDVVLQQHNIAHGRSQVLQQSTYQLLQELCCQHLWQIPEQSQCQAIHKVVHAIILHQQQKQQQQPSSQVSFQQPLQQYPLGQGSFRPSQQNPQAQGSVQPQQLPQFEEIRNLALQTLPAMCNVYIPPYCTITPFGIFGTN

SEQ ID NO: 6 - protein, 268 aa, Homo sapiens
VRVPVPQLQPQNPSQQQPQEQVPLVQQQQFLGQQQPFPPQQPYPQPQPFPSQQPYLQLQPFPQPQLPYSQPQPFRPQQPYPQPQPQYSQPQQPISQQQQQQQQQQQQQQQQQQQQILQQILQQQLIPCMDVVLQQHNIAHGRSQVLQQSTYQLLQELCCQHLWQIPEQSQCQAIHKVVHAIILHQQQKQQQQPSSQVSFQQPLQQYPLGQGSFRPSQQNPQAQGSVQPQQLPQFEEIRNLALQTLPAMCNVYIPPYCTITPFGIFGTN

SEQ ID NO: 7 - protein, 647 aa, Homo sapiens
MWRVCARRAQNVAPWAGLEARWTALQEVPGTPRVTSRSGPAPARRNSVTTGYGGVRALCGWTPSSGATPRNRLLLQLLGSPGRRYYSLPPHQKVPLPSLSPTMQAGTIARWEKKEGDKINEGDLIAEVETDKATVGFESLEECYMAKILVAEGTRDVPIGAIICITVGKPEDIEAFKNYTLDSSAAPTPQAAPAPTPAATASPPTPSAQAPGSSYPPHMQVLLPALSPTMTMGTVQRWEKKVGEKLSEGDLLAEIETDKATIGFEVQEEGYLAKILVPEGTRDVPLGTPLCIIVEKEADISAFADYRPTEVTDLKPQVPPPTPPPVAAVPPTPQPLAPTPSAPCPATPAGPKGRVFVSPLAKKLAVEKGIDLTQVKGTGPDGRITKKDIDSFVPSKVAPAPAAVVPPTGPGMAPVPTGVFTDIPISNIRRVIAQRLMQSKQTIPHYYLSIDVNMGEVLLVRKELNKILEGRSKISVNDFIIKASALACLKVPEANSSWMDTVIRQNHVVDVSVAVSTPAGLITPIVFNAHIKGVETIANDVVSLATKAREGKLQPHEFQGGTFTISNLGMFGIKNFSAIINPPQACILAIGASEDKLVPADNEKGFDVASMMSVTLSCDHRVVDGAVGAQWLAEFRKYLEKPITMLL

SEQ ID NO: 8 - protein, 910 aa, Homo sapiens
MNWHFLRTATVLLIFLVVVEINSEFRIQVRDYNTKNGTIKWHSIRRQKREWIKFAAACREGEDNSKRNPIAKIHSDCAANQQVTYRISGVGIDQPPYGIFIINQKTGEINITSIVDREITPFFIIYCRALNSLGQDLERPLELRVRVLDINDNPPVFSMSTFVGQIEENSNANTLVMRLNATGADEPNNLNSKIAFKIIRQEPSDSPMFIINRNTGEIRTMNNFLDREQYSQYSLAVRGSDRDGGADGMSAECECNIKILDVNDNIPYMEPSSHMVRIEENALSQNLVEIRVIDLDEEFSANWMAVIFFISGNEGGWFDIEMNERTNVGILKVIKPLDYEAVQNLQLSLGVRNKADFHHSIMSQYKVTATAISVTVLNVIEGSVFRPGSKTYVVRSDMGQNYKVGDFVATDLDTGLASTTVRYVMGNNPANLLNVDSKTGVITLRNKVTMEQYEMLNGKYQGTILSIDDALQRTCTGTINIDLQGSGWEKDSEKVTSSQNSGSSTGDSSGGTGGGGRENPSEGDTTTNTGGKTSTDYEDGETQTQSNNNHQELGSNNLSDNVHFGPAGIGLLIMGFLVLGLVPFLLMCCDCGGAPGAGAGFEPVPECSDGAIHSWAVEGPQPLPTDATTVCVPPIPSNNANVIECIDTSGVYTNEYGGREMQDLGGGERTTGFELTEGVKTSGVPEICQEYSGTLRRNSMRECREGGLNMNFMESYFCQKAYAYADEDEGRPSNDCLLIYDIEGVGSPAGSVGCCSFIGEDLDDSFLDTLGPKFKKLADISLGKEVEPDPSWPPESTEPICPQQGTEPIIGGHPPISPHFGTTTVISENTYPSGPGVQHPMPIPDPLGYGNVTVTESYTTSGTLKPTVHVHDNRHASNVVVTERVVGPISGTDLHGMLEMPDLRDGSNVI

SEQ ID NO: 9 - protein, 999 aa, Homo sapiens
MMGLFPRTTGALAIFVVVILVHGELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKDAVCSSSPSVVVSARTLNNRYTGPYTFALEDQPVKLPAVWSITTLNATSALLRAQEQIPPGVYHISLVLTDSQNNRCEMPRSLTLEVCQCDNRGICGTSYPTTSPGTRYGRPHSGRLGPAAIGLLLLGLLLLLLAPLLLLTCDCGAGSTGGVTGGFIPVPDGSEGTIHQWGIEGAHPEDKEITNICVPPVTANGADFMESSEVCTNTYARGTAVEGTSGMEMTTKLGAATESGGAAGFATGTVSGAASGFGAATGVGICSSGQSGTMRTRHSTGGTNKDYADGAISMNFLDSYFSQKAFACAEEDDGQEANDCLLIYDNEGADATGSPVGSVGCCSFIADDLDDSFLDSLGPKFKKLAEISLGVDGEGKEVQPPSKDSGYGIESCGHPIEVQQTGFVKCQTLSGSQGASALSTSGSVQPAVSIPDPLQHGNYLVTETYSASGSLVQPSTAGFDPLLTQNVIVTERVICPISSVPGNLAGPTQLRGSHTMLCTEDPCSRLI

SEQ ID NO: 10 - protein, 323 aa, Homo sapiens
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 11 - protein, 277 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 12 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 13 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 14 - protein, 963 aa, Homo sapiens
MRGASVTVAAVRCGDVAGSCVDGRYNDKDVWKCRCVCDTGTVCDDCDVKDCSGCCCTDATASGGKGKGGDKDVGKGGGAGGRGDRGDKGKGAGRGRDGGTGNGGGGGGGNAAMAGGDKAGGAGVMGMGMGRGGAGAGGGNGGGVSGMGRGGGKGDDGAGKGKAGRGGGARGGTGGVKGHRGYGDGAKGAGAGVKGSGSGNGSGMGRGGRGRTGAGAAGARGNDGGAGGVGAGGGGAGAKGAGTGARGGAGRGGTGSGAGASGNGTDGGAKGSAGAGAGAGGRGGGATGGKGTGGAGKGGKGGAGGAGAGGKRGARGGGVGGGRGAGNRGGDGAGKGAGRGSGAGKGANGDGRGGGARGTGRGDAGGKVGSGAGDGRGGGARGGVMGGKGANGGKAGKGGAGRGGKDGTGAAGGAGAGRGGAGSGGGGGGGKGDGVGAGAGVGRGRGGRGSGAGGRGGTGTDGKGASGAGGAGGGMGRGAAGAGKGDRGDVGKGGAGKDGGRGTGGGAGANGKGVGGAGSAGARGAGRGTGGAGAGGADGGAKGGAGKGDAGAGGSGAGGTGVTGKGARGAGGATGGAAGRVGGSNGNGGGSGKDGKGARGDSGGRAGGGAGGKGGDDGSGAGGGAGRGVGGRGRGGGSGGKGAGASGDRGGVGGTGAGGRGSGADGGRDGAAGVKGDRGTGAVGAGAGGSGAGTGKGDRGAGAGMGSGAGARGGGRGDKGAGGRGKGHRGTGGGGSGDGASGAGSGRGGVGSGKDGANGGGGRGRSGTGAGGNGGGGGDMSAAGGRKGDYMRADAAGGRHDAVDATKSNNSRSGSRKNARTCRDKCHWKSGDYWDNGCTDAMKVCNMTGTCVYNANVKKNWWSSKSKKKHWGTNGGHSYGDDNANTANVMTRSTGSNTYHCKNSAYDAAGNKKAGSNDVRAGNSRTYTAKDGCTKHTGKWGKTVYRSKTSRDAMDGGGVDGVC

SEQ ID NO: 15 - protein, 1419 aa, Homo sapiens
MIRLGAPQSLVLLTLLIAAVLRCQGQDARKLGPKGQKGEPGDIRDIIGPRGPPGPQGPAGEQGPRGDRGDKGEKGAPGPRGRDGEPGTPGNPGPAGPPGPPGPPGLSAGNFAAQMAGGYDEKAGGAQMGVMQGPMGPMGPRGPPGPAGAPGPQGFQGNPGEPGEPGVSGPMGPRGPPGPAGKPGDDGEAGKPGKSGERGLPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGSRGEPGNPGSPGPAGASGNPGTDGIPGAKGSAGAPGIAGAPGFPGPRGPPGPQGATGPLGPKGQAGEPGIAGFKGDQGPKGETGPAGPQGAPGPAGEEGKRGARGEPGGAGPIGPPGERGAPGNRGFPGQDGLAGPKGAPGERGPSGLTGPKGANGDPGRPGEPGLPGARGLTGRPGDAGPQGKVGPSGAPGEDGRPGPPGPQGARGQPGVMGFPGPKGANGEPGKAGEKGLAGAPGLRGLPGKDGETGAAGPPGPSGPAGERGEQGAPGPSGFQGLPGPPGPPGEGGKQGDQGIPGEAGAPGLVGPRGERGFPGERGSPGAQGLQGPRGLPGTPGTDGPKGAAGPDGPPGAQGPPGLQGMPGERGAAGIAGPKGDRGDVGEKGPEGAPGKDGGRGLTGPIGPPGPAGANGEKGEVGPPGPSGSTGARGAPGERGETGPPGPAGFAGPPGADGQPGAKGDQGEAGQKGDAGAPGPQGPSGAPGPQGPTGVTGPKGARGAQGPPGATGFPGAAGRVGPPGANGNPGPAGPPGPAGKDGPKGVRGDSGPPGRAGDPGLQGPAGAPGEKGEPGDDGPSGLDGPPGPQGLAGQRGIVGLPGQRGERGFPGLPGPSGEPGKQGAPGASGDRGPPGPVGPPGLTGPAGEPGREGSPGADGPPGRDGAAGVKGDRGETGALGAPGAPGPPGSPGPAGPTGKQGDRGEAGAQGPMGPSGPAGARGIAGPQGPRGDKGESGEQGERGLKGHRGFTGLQGLPGPPGPSGDQGASGPAGPSGPRGPPGPVGPSGKDGSNGIPGPIGPPGPRGRSGETGPVGPPGSPGPPGPPGPPGPGIDMSAFAGLGQREKGPDPMQYMRADEADSTLRQHDVEVDATLKSLNNQIESIRSPDGSRKNPARTCQDLKLCHPEWKSGDYWIDPNQGCTLDAMKVFCNMETGETCVYPNPATVPRKNWWSSKSKEKKHIWFGETMNGGFHFSYGDGNLAPNTANVQMTFLRLLSTEGSQNITYHCKNSIAYLDEAAGNLKKALLIQGSNDVEMRAEGNSRFTYTALKDGCTKHTGKWGKTVIEYRSQKTSRLPIIDIAPMDIGGAEQEFGVDIGPVCFL

SEQ ID NO: 16 - protein, 216 aa, Homo sapiens
  MOD_RES 5: note = Any amino acid
  MOD_RES 20: note = Any amino acid
  MOD_RES 47: note = Any amino acid
NEIAXAKIDLRQMRTVTPIXMQGGCGSCWALSGVAATESAYLAYGNXSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 17 - protein, 146 aa, Homo sapiens
MMYKILCLSLLVAAVARDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 18 - protein, 406 aa, Homo sapiens
MDKIRYRLVYNRQNTLNRQGTALVQVEAYLNQRKIYLKTNVYLKPECWSREGAQVINHPQSNELNIMLYEYILYLQGIELGYWKRGIPATLSLLKDAVKKKSAVNISFSTFAKSAIDNSDKKQSTKDNLHSTLAVLHDFRSGLDFKDLTYTFLRDFEQYLREKGNAVNTIAKHMRQLRTLVNEAINQGYMHADAYPFRKYKIKQEKGRHEFLTPDELKKLETVEVEEESMRHVLDAFLFCCYTGLRYSDFCQLTPENFIRINGKRWLYFKSVKTGVEIRLPLHLLFESRALGILDRYPDIGSFAALPCNSEVNKQLRKLAGLCGIKKRITYHVSRHTCATLLIHQGVAITTVQKLLGHTSVKTTQIYSEVLSSTIVRDLKNVQKGKRKVKMFPDKGLRTSDFIDNR

SEQ ID NO: 19 - protein, 459 aa, Homo sapiens
MVVQHNLRAMNSNRMLGITQGSLNKSTEKLSSGYKVNRAADDAAGLSISEKMRKQIRGLSQASLNAEDGISAVQTAEGALTEVHDMLQRMNELAVKAANGTNSTSDRQTIQDEVDQLLTEIDRVAETTKFNELYTLKGDEDKVTRYLSAHDAGIEGTLTQGATNATFSMDQLKFGDTIMIAGREYHISGTKAEQAAIITASVKIGQQVTIDGIMYTCSSVSNADKFELKSEDLIAKLDTSSLSIMSVNGKTYYGAGITDDRTVVSSIGAYKLIQKELGLASSIGADGATQASVNAGVDGKTLMKPSFEGKWVFSIDKGSVQVREDIDFSLHVGADADMNNKIAVKIGALDTKGLGIQGLNVKDTTGAAATYAIDSIADAVARISAQRSLLGAVQNRLEHTINNLDNVVENTTAAESQIRDTDMATEMVKYSNNNVLAQAGQSMLAQSNQANQGVLQLLQ

SEQ ID NO: 20 - protein, 460 aa, Homo sapiens
MVVQHNLRAMNSNRMLGITQGSLNKSTEKLSSGYKVNRAADDAAGLSISEKMRKQIRGLSQASLNAEDGISAVQTAEGALTEVHDMLQRMNELAVKAANGTNSTSDRQTIQDEVDQLLTEIDRVAETTKFNELYTLKGDEDKVTRYLSAHDAGIEGTLTQGATNATFSMDQLKFGDTIMIAGREYHISGTQKQQGEIITSSVKIGQQVTIDGIMYTCTATVSNADKFELTKDDLIAKLDTSSLSIMSVNGKTYYGAGITDDRTVVSSIGAYKLIQKELGLASSIGADGSTQASVNAGVDGKTLKKPSFEGKWVFSIDKGSVQVREDIDFSLHVGADADMNNKIAVKIGALDTKGLGIQGLNVKDTTGAAATYAIDSIADAVARISAQRSLLGAVQNRLEHTINNLDNVVENTTAAESQIRDTDMATEMVKYSNNNVLAQAGQSMLAQSNQANQGVLSLLG

SEQ ID NO: 21 - protein, 218 aa, Homo sapiens
MKLNFKGFFKAAGLFPLALMLSGCISYALVSHTAKGSSGKYQSQSDTITGLSQAKDSNGTKGYVFVGESLDYLITDGADDIVKMLNDPALNRHNIQVADDARFVLNAGKKKFTGTISLYYYWNNEEEKALATHYGFACGVQHCTRSLENLKGTIHEKNKNMDYSKVMAFYHPFKVRFYEYYSPRGIPDGVSAALLPVTVTLDIITAPLQFLVVYAVNQ

SEQ ID NO: 22 - protein, 412 aa, Homo sapiens
MGQDQTKQQIEKGLQLYQSNQTEKALQVWTKVLEKSSDLMGRFRVLGCLVTAHSEMGRYKEMLKFAVVQIDTARELEDADFLLESYLNLARSNEKLCEFHKTISYCKTCLGLPGTRAGAQLGGQVSLSMGNAFLGLSVFQKALESFEKALRYAHNNDDAMLECRVCCSLGSFYAQVKDYEKALFFPCKAAELVNNYGKGWSLKYRAMSQYHMAVAYRLLGRLGSAMECCEESMKIALQHGDRPLQALCLLCFADIHRSRGDLETAFPRYDSAMSIMTEIGNRLGQVQALLGVAKCWVARKALDKALDAIERAQDLAEEVGNKLSQLKLHCLSESIYRSKGLQRELRAHVVRFHECVEETELYCGLCGESIGEKNSRLQALPCSHIFHLRCLQNNGTRSCPNCRRSSMKPGFV

SEQ ID NO: 23 - protein, 493 aa, Homo sapiens
MARAPLGVLLLLGLLGRGVGKNEELRLYHHLFNNYDPGSRPVREPEDTVTISLKVTLTNLISLNEKEETLTTSVWIGIDWQDYRLNYSKDDFGGIETLRVPSELVWLPEIVLENNIDGQFGVAYDANVLVYEGGSVTWLPPAIYRSVCAVEVTYFPFDWQNCSLIFRSQTYNAEEVEFTFAVDNDGKTINKIDIDTEAYTENGEWAIDFCPGVIRRHHGGATDGPGETDVIYSLIIRRKPLFYVINIIVPCVLISGLVLLAYFLPAQAGGQKCTVSINVLLAQTVFLFLIAQKIPETSLSVPLLGRFLIFVMVVATLIVMNCVIVLNVSQRTPTTHAMSPRLRHVLLELLPRLLGSPPPPEAPRAASPPRRASSVGLLLRAEELILKKPRSELVFEGQRHRQGTWTAAFCQSLGAAAPEVRCCVDAVNFVAESTRDQEATGEEVSDWVRMGNALDNICFWAALVLFSVGSSLIFLGAYFNRVPDLPYAPCIQP

SEQ ID NO: 24 - protein, 482 aa, Homo sapiens
MEPWPLLLLFSLCSAGLVLGSEHETRLVAKLFKDYSSVVRPVEDHRQVVEVTVGLQLIQLINVDEVNQIVTTNVRLKQGDMVDLPRPSCVTLGVPLFSHLQNEQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPLYFIVNVIIPCLLFSFLTGLVFYLPTDSGEKMTLSISVLLSLTVFLLVIVELIPSTSSAVPLIGKYMLFTMVFVIASIIITVIVINTHHRSPSTHVMPNWVRKVFIDTIPNIMFFSTMKRPSREKQDKKIFTEDIDISDISGKPGPPPMGFHSPLIKHPEVKSAIEGIKYIAETMKSDQESNNAAAEWKYVAMVMDHILLGVFMLVCIIGTLAVFAGRLIELNQQG

SEQ ID NO: 25 - protein, 517 aa, Homo sapiens
MHGGQGPLLLLLLLAVCLGAQGRNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIYWLPPAIFRSACSISVTYFPFDWQNCSLIFQSQTYSTNEIDLQLSQEDGQTIEWIFIDPEAFTENGEWAIQHRPAKMLLDPAAPAQEAGHQKVVFYLLIQRKPLFYVINIIAPCVLISSVAILIHFLPAKAGGQKCTVAINVLLAQTVFLFLVAKKVPETSQAVPLISKYLTFLLVVTILIVVNAVVVLNVSLRSPHTHSMARGVRKVFLRLLPQLLRMHVRPLAPAAVQDTQSRLQNGSSGWSITTGEEVALCLPRSELLFQQWQRQGLVAAALEKLEKGPELGLSQFCGSLKQAAPAIQACVEACNLIACARHQQSHFDNGNEEWFLVGRVLDRVCFLAMLSLFICGTAGIFLMAHYNRVPALPFPGDPRPYLPSPD

SEQ ID NO: 26 - protein, 501 aa, Homo sapiens
MTPGALLMLLGALGAPLAPGVRGSEAEGRLREKLFSGYDSSVRPAREVGDRVRVSVGLILAQLISLNEKDEEMSTKVYLDLEWTDYRLSWDPAEHDGIDSLRITAESVWLPDVVLLNNNDGNFDVALDISVVVSSDGSVRWQPPGIYRSSCSIQVTYFPFDWQNCTMVFSSYSYDSSEVSLQTGLGPDGQGHQEIHIHEGTFIENGQWEIIHKPSRLIQPPGDPRGGREGQRQEVIFYLIIRRKPLFYLVNVIAPCILITLLAIFVFYLPPDAGEKMGLSIFALLTLTVFLLLLADKVPETSLSVPIIIKYLMFTMVLVTFSVILSVVVLNLHHRSPHTHQMPLWVRQIFIHKLPLYLRLKRPKPERDLMPEPPHCSSPGSGWGRGTDEYFIRKPPSDFLFPKPNRFQPELSAPDLRRFIDGPNRAVALLPELREVVSSISYIARQLQEQEDHDALKEDWQFVAMVVDRLFLWTFIIFTSVGTLVIFLDATYHLPPPDPFP

SEQ ID NO: 27 - protein, 517 aa, Homo sapiens
MEGPVLTLGLLAALAVCGSWGLNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSCNVLVYHYGFVYWLPPAIFRSSCPISVTYFPFDWQNCSLKFSSLKYTAKEITLSLKQDAKENRTYPVEWIIIDPEGFTENGEWEIVHRPARVNVDPRAPLDSPSRQDITFYLIIRRKPLFYIINILVPCVLISFMVNLVFYLPADSGEKTSVAISVLLAQSVFLLLISKRLPATSMAIPLIGKFLLFGMVLVTMVVVICVIVLNIHFRTPSTHVLSEGVKKLFLETLPELLHMSRPAEDGPSPGALVRRSSSLGYISKAEEYFLLKSRSDLMFEKQSERHGLARRLTTARRPPASSEQAQQELFNELKPAVDGANFIVNHMRDQNNYNEEKDSWNRVARTVDRLCLFVVTPVMVVGTAWIFLQGVYNQPPPQPFPGDPYSYNVQDKRFI

SEQ ID NO: 28 - protein, 933 aa, Homo sapiens
MRALAVLSVTLVMACTEAFFPFISRGKELLWGKPEESRVSSVLEESKRLVDTAMYATMQRNLKKRGILSPAQLLSFSKLPEPTSGVIARAAEIMETSIQAMKRKVNLKTQQSQHPTDALSEDLLSIIANMSGCLPYMLPPKCPNTCLANKYRPITGACNNRDHPRWGASNTALARWLPPVYEDGFSQPRGWNPGFLYNGFPLPPVREVTRHVIQVSNEVVTDDDRYSDLLMAWGQYIDHDIAFTPQSTSKAAFGGGADCQMTCENQNPCFPIQLPEEARPAAGTACLPFYRSSAACGTGDQGALFGNLSTANPRQQMNGLTSFLDASTVYGSSPALERQLRNWTSAEGLLRVHARLRDSGRAYLPFVPPRAPAACAPEPGIPGETRGPCFLAGDGRASEVPSLTALHTLWLREHNRLAAALKALNAHWSADAVYQEARKVVGALHQIITLRDYIPRILGPEAFQQYVGPYEGYDSTANPTVSNVFSTAAFRFGHATIHPLVRRLDASFQEHPDLPGLWLHQAFFSPWTLLRGGGLDPLIRGLLARPAKLQVQDQLMNEELTERLFVLSNSSTLDLASINLQRGRDHGLPGYNEWREFCGLPRLETPADLSTAIASRSVADKILDLYKHPDNIDVWLGGLAENFLPRARTGPLFACLIGKQMKALRDGDWFWWENSHVFTDAQRRELEKHSLSRVICDNTGLTRVPMDAFQVGKFPEDFESCDSITGMNLEAWRETFPQDDKCGFPESVENGDFVHCEESGRRVLVYSCRHGYELQGREQLTCTQEGWDFQPPLCKDVNECADGAHPPCHASARCRNTKGGFQCLCADPYELGDDGRTCVDSGRLPRVTWISMSLAALLIGGFAGLTSTVICRWTRTGTKSTLPISETGGGTPELRCGKHQAVGTSPQRAAAQDSEQESAGMEGRDTHRLPRAL

SEQ ID NO: 29 - protein, 764 aa, Homo sapiens
MRPADLLQLVLLLDLPRDLGGMGCSSPPCECHQEEDFRVTCKDIQRIPSLPPSTQTLKLIETHLRTIPSHAFSNLPNISRIYVSIDVTLQQLESHSFYNLSKVTHIEIRNTRNLTYIDPDALKELPLLKFLGIFNTGLKMFPDLTKVYSTDIFFILEITDNPYMTSIPVNAFQGLCNETLTLKLYNNGFTSVQGYAFNGTKLDAVYLNKNKYLTVIDKDAFGGVYSGPSLLDVSQTSVTALPSKGLEHLKELIARNTWTLKKLPLSLSFLHLTRADLSYPSHCCAFKNQKKIRGILESLMCNESSMQSLRQRKSVNALNSPLHQEYEENLGDSIVGYKEKSKFQDTHNNAHYYVFFEEQEDEIIGFGQELKNPQEETLQAFDSHYDYTICGDSEDMVCTPKSDEFNPCEDIMGYKFLRIVVWFVSLLALLGNVFVLLILLTSHYKLNVPRFLMCNLAFADFCMGMYLLLIASVDLYTHSEYYNHAIDWQTGPGCNTAGFFTVFASELSVYTLTVITLERWYAITFAMRLDRKIRLRHACAIMVGGWVCCFLLALLPLVGISSYAKVSICLPMDTETPLALAYIVFVLTLNIVAFVIVCCCYVKIYITVRNPQYNPGDKDTKIAKRMAVLIFTDFICMAPISFYALSAILNKPLITVSNSKILLVLFYPLNSCANPFLYAIFTKAFQRDVFILLSKFGICKRQAQAYRGQRVPPKNSTDIQVQKVTHDMRQGLHNMEDVYELIENSHLTPKKQGQISEEYMQTVL

SEQ ID NO: 30 - protein, 770 aa, Homo sapiens
PRVPWKMRPADLLQLVLLLDLPRDLGGMGCSSPPCECHQEEDFRVTCKDIQRIPSLPPSTQTLKLIETHLRTIPSHAFSNLPNISRIYVSIDVTLQQLESHSFYNLSKVTHIEIRNTRNLTYIDPDALKELPLLKFLGIFNTGLKMFPDLTKVYSTDIFFILEITDNPYMTSIPVNAFQGLCNETLTLKLYNNGFTSVQGYAFNGTKLDAVYLNKNKYLTVIDKDAFGGVYSGPSLLDVSQTSVTALPSKGLEHLKELIARNTWTLKKLPLSLSFLHLTRADLSYPSHCCAFKNQKKIRGILESLMCNESSMQSLRQRKSVNALNSPLHQEYEENLGDSIVGYKEKSKFQDTHNNAHYYVFFEEQEDEIIGFGQELKNPQEETLQAFDSHYDYTICGDSEDMVCTPKSDEFNPCEDIMGYKFLRIVVWFVSLLALLGNVFVLLILLTSHYKLNVPRFLMCNLAFADFCMGMYLLLIASVDLYTHSEYYNHAIDWQTGPGCNTAGFFTVFASELSVYTLTVITLERWYAITFAMRLDRKIRLRHACAIMVGGWVCCFLLALLPLVGISSYAKVSICLPMDTETPLALAYIVFVLTLNIVAFVIVCCCYVKIYITVRNPQYNPGDKDTKIAKRMAVLIFTDFICMAPISFYALSAILNKPLITVSNSKILLVLFYPLNSCANPFLYAIFTKAFQRDVFILLSKFGICKRQAQAYRGQRVPPKNSTDIQVQKVTHDMRQGLHNMEDVYELIENSHLTPKKQGQISEEYMQTVL

SEQ ID NO: 31 - protein, 411 aa, Homo sapiens
MRPADLLQLVLLLDLPRDLGGMGCSSPPCECHQEEDFRVTCKDIQRIPSLPPSTQTLKLIETHLRIVVWFVSLLALLGNVFVLLILLTSHYKLNVPRFLMCNLAFADFCMGMYLLLIASVDLYTHSEYYNHAIDWQTGPGCNTAGFFTVFASELSVYTLTVITLERWYAITFAMRLDRKIRLRHACAIMVGGWVCCFLLALLPLVGISSYAKVSICLPMDTETPLALAYIVFVLTLNIVAFVIVCCCYVKIYITVRNPQYNPGDKDTKIAKRMAVLIFTDFICMAPISFYALSAILNKPLITVSNSKILLVLFYPLNSCANPFLYAIFTKAFQRDVFILLSKFGICKRQAQAYRGQRVPPKNSTDIQVQKVTHEMRQGLHNMEDVYELIENSHLTPKKQGQISEEYMQTVL

SEQ ID NO: 32 - protein, 345 aa, Homo sapiens
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 33 - protein, 102 aa, Homo sapiens
SGRGKGGKGLGKGGAKRHRKVLRDNIQGITKPAIRRLARRGGVKRISGLIYEETRGVLKVFLENVIRDAVTYTEHAKRKTVTAMDVVYALKRQGRTLYGFGG

SEQ ID NO: 34 - protein, 125 aa, Homo sapiens
PEPAKSAPAPKKGSKKAVTKAQKKDGKKRKRSRKESYSVYVYKVLKQVHPDTGISSKAMGIMNSFVNDIFERIASEASRLAHYNKRSTITSREIQTAVRLLLPGELAKHAVSEGTKAVTKYTSSK

SEQ ID NO: 35 - protein, 194 aa, Homo sapiens
MTENSTSAPAAKPKRAKASKKSTDHPKYSDMIVAAIQAEKNRAGSSRQSIQKYIKSHYKVGENADSQIKLSIKRLVTTGVLKQTKGVGASGSFRLAKGDEPKRSVAFKKTKKEVKKVATPKKAAKPKKAASKAPSKKPKATPVKKAKKKPAATPKKAKKPKVVKVKPVKASKPKKAKTVKPKAKSSAKRASKKK

SEQ ID NO: 36 - protein, 4505 aa, Homo sapiens
MGPRKPALRTPLLLLFLLLFLDTSVWAQDEVLENLSFSCPKDATRFKHLRKYVYNYEAESSSGVQGTADSRSATKINCKVELEVPQICGFIMRTNQCTLKEVYGFNPEGKALMKKTKNSEEFAAAMSRYELKLAIPEGKQIVLYPDKDEPKYILNIKRGIISALLVPPETEEDQQELFLDTVYGNCSTQVTVNSRKGTVPTEMSTERNLQQCDGFQPISTSVSPLALIKGLVHPLSTLISSSQTCQYTLDPKRKHVSEAVCDEQHLFLPFSYKNKYGIMTRVTQKLSLEDTPKINSRFFSEGTNRMGLAFESTKSTSSPKQADAVLKTLQELKKLSISEQNAQRANLFNKLVTELRGLTGEAITSLLPQLIEVSSPITLQALVQCGQPQCYTHILQWLKTEKAHPLLVDIVTYLMALIPNPSTQRLQEIFNTAKEQQSRATLYALSHAVNSYFDVDHSRSPVLQDIAGYLLKQIDNECTGNEDHTFLILRVIGNMGRTMEQVMPALKSSVLSCVRSTKPSLLIQKAALQALRKMELEDEVRTILFDTFVNGVAPVEKRLAAYLLLMKNPSSSDINKIAQLLQWEQSEQVKNFVASHIANILNSEELYVQDLKVLIKNALENSQFPTIMDFRKFSRNYQISKSASLPMFDPVSVKIEGNLIFDPSSYLPRESLLKTTLTVFGLASLDLFEIGLEGKGFEPTLEALFGKQGFFPDSVNKALYWVNGRVPDGVSKVLVDHFGYTTDGKHEQDMVNGIMPIVDKLIKDLKSKEIPEARAYLRILGKELSFVRLQDLQVLGKLLLSGAQTLQGIPQMVVQAIREGSKNDLFLHYIFMDNAFELPTGAGLQLQVSSSGVFTPGIKAGVRLELANIQAELVAKPSVSLEFVTNMGIIIPDFAKSSVQMNTNFFHESGLEARVALKAGQLKVIIPSPKRPVKLFSGSNTLHLVSTTKTEVIPPLVENRQSWSTCKPLFTGMNYCTTGAYSNASSTESASYYPLTGDTRYELELRPTGEVEQYSATATYELLKEDKSLVDTLKFLVQAEGVQQSEATVLFKYNRRSRTLSSEVLIPGFDVNFGTILRVNDESAKDKNTYKLILDIQNKKITEVSLVGHLSYDKKGDGKIKGVVSIPRLQAEARSEVHTHWSSTKLLFQMDSSATAYGSTISKRVTWRYDNEIIEFDWNTGTNVDTKKVASNFPVDLSHYPRMLHEYANGLLDHRVPQTDVTFRDMGSKLIVATNTWLQMATRGLPYPQTLQDHLNSLSELNLLKMGLSDFHIPDNLFLKTDGRVKYTMNRNKINIDIPLPLGGKSSKDLKMPESVRTPALNFKSVGFHLPSREVQVPTFTIPKTHQLQVPLLGVLDLSTNVYSNLYNWSASYTGGNTSRDHFSLQAQYRMKTDSVVDLFSYSVQGSGETTYDSKNTFTLSCDGSLHHKFLDSKFKVSHVEKFGNSPVSKGLLTFETSSALGPQMSATVHLDSKKKQHLYVKDIKVDGQFRASSFYAQGKYGLSCERDVTTGQLSGESNMRFNSTYFQGTNQIVGMYQDGALSITSTSDLQDGIFKNTASLKYENYELTLKSDSSGQYENFAASNKLDVTFSTQSALLRSEHQANYKSLRLVTLLSGSLTSQGVELNADILGTDKINTGAHKATLKIARDGLSTSATTNLKYSPLLLENELNAELGLSGASMKLSTNGRFKEHHAKFSLDGRAALTEVSLGSIYQAMILGADSKNIFNFKLSREGLRLSNDLMGSYAEMKLDHTHSLNIAGLSLDFFSKMDNIYSGDKFYKQNFNLQLQPYSFITTLSNDLRYGALDLTNNGRFRLEPLKLNVGGNFKGTYQNNELKHIYTISYTDLVVASYRADTVAKVQGVEFSHRLNADIEGLTSSVDVTTSYNSDPLHFNNVFHFSLAPFTLGIDTHTSGDGKLSFWGEHTGQLYSKFLLKAEPLALIVSHDYKGSTSHSLPYESSISTALEHTVSALLTPAEQTSTWKFKTKLNDKVYSQDFEAYNTKDKIGVELSGRADLSGLYSPIKLPFFYSEPVNVLNGLEVNDAVDKPQEFTIIAVVKYDKNQDVHTINLPFFKSLPDYLERNRRGMISLLEAMRGELQRLSVDQFVRKYRAALSRLPQQIHHYLNASDWERQVAGAKEKITSFMENYRITDNDVLIAIDSAKINFNEKLSQLETYAIQFDQYIKDNYDPHDLKRTIAEIIDRIIEKLKILDEQYHIRVNLAKSIHNLYLFVENVDLNQVSSSNTSWIQNVDSNYQVRIQIQEKLQQLRTQIQNIDIQQLAAEVKRQMDAIDVTMHLDQLRTAILFQRISDIIDRVKYFVMNLIEDFKVTEKINTFRVIVRELIEKYEVDQHIQVLMDKSVELAHRYSLSEPLQKLSNVLQRIEIKDYYEKLVGFIDDTVEWLKALSFKNTIEELNRLTDMLVKKLKAFDYHQFVDKTNSKIREMTQRINAEIQALKLPQKMEALKLLVEDFKTTVSNSLERLKDTKVTVVIDWLQDILTQMKDHFQDTLEDVRDRIYQMDIQRELEHFLSLVNQVYSTLVTYMSDWWTLTAKNITDFAEQYSIQNWAESIKVLVEQGFIVPEMQTFLWTMPAFEVSLRALQEGNFQTPVFIVPLTDLRIPSIRINFKMLKNIKIPLRFSTPEFTLLNTFHVHSFTIDLLEIKAKIIRTIDQILSSELQWPLPEMYLRDLDVVNIPLARLTLPDFHVPEITIPEFTIPNVNLKDLHVPDLHIPEFQLPHLSHTIEIPAFGKLHSILKIQSPLFILDANANIQNVTTSGNKAEIVASVTAKGESQFEALNFDFQAQAQFLELNPHPPVLKESMNFSSKHVRMEHEGEIVFDGKAIEGKSDTVASLHTEKNEVEFNNGMTVKVNNQLTLDSHTKYFHKLSVPRLDFSSKASLNNEIKTLLEAGHVALTSSGTGSWNWACPNFSDEGIHSSQISFTVDGPIAFVGLSNNINGKHLRVIQKLTYESGFLNYSKFEVESKVESQHVGSSILTANGRALLKDAKAEMTGEHNANLNGKVIGTLKNSLFFSAQPFEITASTNNEGNLKVGFPLKLTGKIDFLNNYALFLSPRAQQASWQASTRFNQYKYNQNFSAINNEHNIEASIGMNGDANLDFLNIPLTIPEINLPYTEFKTPLLKDFSIWEETGLKEFLKTTKQSFDLSVKAQYKKNSDKHSIVVPLGMFYEFILNNVNSWDRKFEKVRNNALHFLTTSYNEAKIKVDKYKTENSLNQPSGTFQNHGYTIPVVNIEVSPFAVETLASSHVIPTAISTPSVTIPGPNIMVPSYKLVLPPLELPVFHGPGNLFKFFLPDFKGFNTIDNIYIPAMGNFTYDFSFKSSVITLNTNAGLYNQSDIVAHFLSSSSFVTDALQYKLEGTSRLMRKRGLKLATAVSLTNKFVKGSHDSTISLTKKNMEASVRTTANLHAPIFSMNFKQELNGNTKSKPTVSSSIELNYDFNSSKLHSTATGGIDHKFSLESLTSYFSIESFTKGNIKSSFLSQEYSGSVANEANVYLNSKGTRSSVRLQGASKVDGIWNVEVGENFAGEATLQRIYTTWEHNMKNHLQVYSYFFTKGKQTCRATLELSPWTMSTLLQVHVSQLSSLLDLHHFDQEVILKANTKNQKISWKGGVQVESRVLQHNAQFSNDQEEIRLDLAGSLDGQLWDLEAIFLPVYGKSLQELLQMDGKRQYLQASTSLLYTKNPNGYLLSLPVQELADRFIIPGIKLNDFSGVKIYKKLSTSPFALNLTMLPKVKFPGIDLLTQYSTPEGSSVPIFEATIPEIHLTVSQFTLPKSLPVGNTVFDLNKLANMIADVDLPSVTLPEQTIVIPPLEFSVPAGIFIPFFGELTARAGMASPLYNVTWSAGWKTKADHVETFLDSMCTSTLQFLEYALKVVETHKIEEDLLTYNIKGTLQHCDFNVEYNEDGLFKGLWDWQGEAHLDITSPALTDFHLYYKEDKTSLSASAASSTIGTVGLDSSTDDQSVELNVYFHPQSPPEKKLSIFKTEWRYKESDGERYIKINWEEEAASRLLGSLKSNVPKASKAIYDYANKYHLEYVSSELRKSLQVNAEHARRMVDEMNMSFQRVARDTYQNLYEEMLAQKSLSIPENLKKRVLDSIVHVTQKYHMAVMWLMDSFIHFLKFNRVQFPGYAGTYTVDELYTIVMKETKKSLSQLFNGLGNLLSYVQNQVEKSRLINDITFKCPFFSKPCKLKDLILIFREELNILSNIGQQDIKFTTILSSLQGFLERVLDIIEEQIKCLKDNESTCVADHINMVFKIQVPYAFKSLREDIYFVLGEFNDFLQSILQEGSYKLQQVHQYMKALREEYFDPSMVGWTVKYYEIEENMVELIKTLLVSFRDVYSEYSVTAADFASKMSTQVEQFVSRDIREYLSMLTDINGKWMEKIAELSIVAKETMKSWVTAVAKIMSDYPQQFHSNLQDFSDQLSSYYEKFVGESTRLIDLSIQNYHVFLRYITELLRKLQVATANNVSPYIKLAQGELMITF

SEQ ID NO: 37 - protein, 317 aa, Homo sapiens [sequence omitted]

SEQ ID NO: 38 - protein, 1480 aa, Homo sapiens
MKPRAECCSPKFWLVLAVLAVSGSRARSQKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQEDDHLSIVTLEEAPFVIVESVDPLSGTCMRNTVPCQKRIVTENKTDEEPGYIKKCCKGFCIDILKKISKSVKFTYDLYLVTNGKHGKKINGTWNGMIGEVVMKRAYMAVGSLTINEERSEVVDFSVPFIETGISVMVSRSNGTVSPSAFLEPFSADVWVMMFVMLLIVSAVAVFVFEYFSPVGYNRCLADGREPGGPSFTIGKAIWLLWGLVFNNSVPVQNPKGTTSKIMVSVWAFFAVIFLASYTANLAAFMIQEEYVDQVSGLSDKKFQRPNDFSPPFRFGTVPNGSTERNIRNNYAEMHAYMGKFNQRGVDDALLSLKTGKLDAFIYDAAVLNYMAGRDEGCKLVTIGSGKVFASTGYGIAIQKDSGWKRQVDLAILQLFGDGEMEELEALWLTGICHNEKNEVMSSQLDIDNMAGVFYMLGAAMALSLITFICEHLFYWQFRHCFMGVCSGKPGMVFSISRGIYSCIHGVAIEERQSVMNSPTATMNNTHSNILRLLRTAKNMANLSGVNGSPQSALDFIRRESSVYDISEHRRSFTHSDCKSYNNPPCEENLFSDYISEVERTFGNLQLKDSNVYQDHYHHHHRPHSIGSASSIDGLYDCDNPPFTTQSRSISKKPLDIGLPSSKHSQLSDLYGKFSFKSDRYSGHDDLIRSDVSDISTHTVTYGNIEGNAAKRRKQQYKDSLKKRPASAKSRREFDEIELAYRRRPPRSPDHKRYFRDKEGLRDFYLDQFRTENSPHWEHVDLTDIYKERSDDFKRDSVSGGGPCTNRSHIKHGTGDKHGVVSGVPAPWEKNLTNVEWEDRSGGNFCRSCPSKLHNYSTTVTGQNSGRQACIRCEACKKAGNLYDISEDNSLQELDQPAAPVAVTSNASTTKYPQSPTNSKAQKKNRNKLRRQHSYDTFVDLQKEEALAPRSVSLKDKGRFMDGSPYAHMFEMSAGESTFANNKSSVPTAGHHHHNNPGGGYLSKSLYPDRVTQNPFIPTFGDDQCLLHGSKSYFFRQPTVAGASKARPDFRALVTNKPVSALHGAVPARFQKDICIGNQSNPCVPNNKNPRAFNGSSNGHVYEKLSSIESDV

SEQ ID NO: 39 - protein, 653 aa, Homo sapiens
MEVAMVSAESSGCNSHMPYGYAAQARARERERLAHSRAAAAAAVAAATAAVEGSGGSGGGSHHHHQSRGACTSHDPQSSRGSRRRRRQRSEKKKAHYRQSSFPHCSDLMPSGSEEKILRELSEEEEDEEEEEEEEEEGRFYYSEDDHGDECSYTDLLPQDEGGGGYSSVRYSDCCERVVINVSGLRFETQMKTLAQFPETLLGDPEKRTQYFDPLRNEYFFDRNRPSFDAILYYYQSGGRLKRPVNVPFDIFTEEVKFYQLGEEALLKFREDEGFVREEEDRALPENEFKKQIWLLFEYPESSSPARGIAIVSVLVILISIVIFCLETLPEFRDDRDLVMALSAGGHGGLLNDTSAPHLENSGHTIFNDPFFIVETVCIVWFSFEFVVRCFACPSQALFFKNIMNIIDIVSILPYFITLGTDLAQQQGGGNGQQQQAMSFAILRIIRLVRVFRIFKLSRHSKGLQILGHTLRASMRELGLLIFFLFIGVILFSSAVYFAEADEPTTHFQSIPDAFWWAVVTMTTVGYGDMKPITVGGKIVGSLCAIAGVLTIALPVPVIVSNFNYFYHRETENEEQTQLTQNAVSCPYLPSNLLKKFRSSTSSSLGDKSEYLEMEEGVKESLCAKEEKCQGKGDDSETDKNNCSNAKAVETDV

SEQ ID NO: 40 - protein, 786 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 41 - protein, 405 aa, Homo sapiens
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESYQDANLVFEEFARHNLKDAGEAEEGKRDKNDVDE

SEQ ID NO: 42 - protein, 1976 aa, Homo sapiens
MAQRTGLEDPERYLFVDRAVIYNPATQADWTAKKLVWIPSERHGFEAASIKEERGDEVMVELAENGKKAMVNKDDIQKMNPPKFSKVEDMAELTCLNEASVLHNLKDRYYSGLIYTYSGLFCVVINPYKNLPIYSENIIEMYRGKKRHEMPPHIYAISESAYRCMLQDREDQSILCTGESGAGKTENTKKVIQYLAHVASSHKGRKDHNIPGELERQLLQANPILESFGNAKTVKNDNSSRFGKFIRINFDVTGYIVGANIETYLLEKSRAVRQAKDERTFHIFYQLLSGAGEHLKSDLLLEGFNNYRFLSNGYIPIPGQQDKDNFQETMEAMHIMGFSHEEILSMLKVVSSVLQFGNISFKKERNTDQASMPENTVAQKLCHLLGMNVMEFTRAILTPRIKVGRDYVQKAQTKEQADFAVEALAKATYERLFRWLVHRINKALDRTKRQGASFIGILDIAGFEIFELNSFEQLCINYTNEKLQQLFNHTMFILEQEEYQREGIEWNFIDFGLDLQPCIDLIERPANPPGVLALLDEECWFPKATDKTFVEKLVQEQGSHSKFQKPRQLKDKADFCIIHYAGKVDYKADEWLMKNMDPLNDNVATLLHQSSDRFVAELWKDVDRIVGLDQVTGMTETAFGSAYKTKKGMFRTVGQLYKESLTKLMATLRNTNPNFVRCIIPNHEKRAGKLDPHLVLDQLRCNGVLEGIRICRQGFPNRIVFQEFRQRYEILTPNAIPKGFMDGKQACERMIRALELDPNLYRIGQSKIFFRAGVLAHLEEERDLKITDIIIFFQAVCRGYLARKAFAKKQQQLSALKVLQRNCAAYLKLRHWQWWRVFTKVKPLLQVTRQEEELQAKDEELLKVKEKQTKVEGELEEMERKHQQLLEEKNILAEQLQAETELFAEAEEMRARLAAKKQELEEILHDLESRVEEEEERNQILQNEKKKMQAHIQDLEEQLDEEEGARQKLQLEKVTAEAKIKKMEEEILLLEDQNSKFIKEKKLMEDRIAECSSQLAEEEEKAKNLAKIRNKQEVMISDLEERLKKEEKTRQELEKAKRKLDGETTDLQDQIAELQAQIDELKLQLAKKEEELQGALARGDDETLHKNNALKVVRELQAQIAELQEDFESEKASRNKAEKQKRDLSEELEALKTELEDTLDTTAAQQELRTKREQEVAELKKALEEETKNHEAQIQDMRQRHATALEELSEQLEQAKRFKANLEKNKQGLETDNKELACEVKVLQQVKAESEHKRKKLDAQVQELHAKVSEGDRLRVELAEKASKLQNELDNVSTLLEEAEKKGIKFAKDAASLESQLQDTQELLQEETRQKLNLSSRIRQLEEEKNSLQEQQEEEEEARKNLEKQVLALQSQLADTKKKVDDDLGTIESLEEAKKKLLKDAEALSQRLEEKALAYDKLEKTKNRLQQELDDLTVDLDHQRQVASNLEKKQKKFDQLLAEEKSISARYAEERDRAEAEAREKETKALSLARALEEALEAKEEFERQNKQLRADMEDLMSSKDDVGKNVHELEKSKRALEQQVEEMRTQLEELEDELQATEDAKLRLEVNMQAMKAQFERDLQTRDEQNEEKKRLLIKQVRELEAELEDERKQRALAVASKKKMEIDLKDLEAQIEAANKARDEVIKQLRKLQAQMKDYQRELEEARASRDEIFAQSKESEKKLKSLEAEILQLQEELASSERARRHAEQERDELADEITNSASGKSALLDEKRRLEARIAQLEEELEEEQSNMELLNDRFRKTTLQVDTLNAELAAERSAAQKSDNARQQLERQNKELKAKLQELEGAVKSKFKATISALEAKIGQLEEQLEQEAKERAAANKLVRRTEKKLKEIFMQVEDERRHADQYKEQMEKANARMKQLKRQLEEAEEEATRANASRRKLQRELDDATEANEGLSREVSTLKNRLRRGGPISFSSSRSGRRQLHLEGASLELSDDDTESKTSDVNETQPPQSE

SEQ ID NO: 43 - protein, 1939 aa, Homo sapiens [sequence omitted]

SEQ ID NO: 44 - protein, 573 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 45 - protein, 15 aa, Homo sapiens
KISVSLPLSLSQSVC

SEQ ID NO: 46 - protein, 14 aa, Homo sapiens
QLSKDTSVLTFTFC

SEQ ID NO: 47 - protein, 17 aa, Homo sapiens
CSDAHPGDSSGDSSGLN

SEQ ID NO: 48 - protein, 15 aa, Homo sapiens
RGEVRQFTLRHWLKV

SEQ ID NO: 49 - protein, 13 aa, Homo sapiens
GDYLNDEALWNKC

SEQ ID NO: 50 - protein, 15 aa, Homo sapiens
GKVIDDNDHLSQEIC

SEQ ID NO: 51 - protein, 14 aa, Homo sapiens
LMANSTWGYPFHDG

SEQ ID NO: 52 - protein, 14 aa, Homo sapiens
LNVVPWNLTLFSIL

SEQ ID NO: 53 - protein, 12 aa, Homo sapiens
THSFTAFKRHVC

SEQ ID NO: 54 - protein, 13 aa, Homo sapiens
NLSLPPSLSLSIC

SEQ ID NO: 55 - protein, 16 aa, Homo sapiens
ERPSSVLTIYDIGIQC

SEQ ID NO: 56 - protein, 15 aa, Homo sapiens
CYQQYTNLQERPSSV

SEQ ID NO: 57 - protein, 15 aa, Homo sapiens [sequence omitted]

SEQ ID NO: 58 - protein, 17 aa, Homo sapiens
CSRKKRADKKENGTKLL

SEQ ID NO: 59 - protein, 9 aa, Homo sapiens
FLLVLGFII

SEQ ID NO: 60 - protein, 9 aa, Homo sapiens
VLPSVAMFL

SEQ ID NO: 61 - protein, 9 aa, Homo sapiens
LVLGFIIAL

SEQ ID NO: 62 - protein, 9 aa, Homo sapiens
KVVTSSFVV

SEQ ID NO: 63 - protein, 9 aa, Homo sapiens
LVPGTKFYI

SEQ ID NO: 64 - protein, 9 aa, Homo sapiens
LLPIRTLPL

SEQ ID NO: 65 - protein, 9 aa, Homo sapiens
YLVKKGTAT

SEQ ID NO: 66 - protein, 9 aa, Homo sapiens
SLFAETIWV

SEQ ID NO: 67 - protein, 9 aa, Homo sapiens
MLIAMYFYT

SEQ ID NO: 68 - protein, 9 aa, Homo sapiens
LMWTLPVML

SEQ ID NO: 69 - protein, 9 aa, Homo sapiens
MLIVYIFEC

SEQ ID NO: 70 - protein, 9 aa, Homo sapiens
YIFECASCI

SEQ ID NO: 71 - protein, 9 aa, Homo sapiens
LVLMLIVYI

SEQ ID NO: 72 - protein, 9 aa, Homo sapiens
ALCRRRSMV

SEQ ID NO: 73 - protein, 9 aa, Homo sapiens
LLSGLSLFA

SEQ ID NO: 74 - protein, 9 aa, Homo sapiens
FLLVVGLIV

SEQ ID NO: 75 - protein, 9 aa, Homo sapiens
LVVGLIVAL

SEQ ID NO: 76 - protein, 9 aa, Homo sapiens
KVVKSDFVV

SEQ ID NO: 77 - protein, 9 aa, Homo sapiens
TLPVQTLPL

SEQ ID NO: 78 - protein, 9 aa, Homo sapiens
DLHVISNDV

SEQ ID NO: 79 - protein, 9 aa, Homo sapiens
VLVHPQWVL

SEQ ID NO: 80 - protein, 9 aa, Homo sapiens
FLRPGDDSS

SEQ ID NO: 81 - protein, 9 aa, Homo sapiens
ALGTTCYAS

SEQ ID NO: 82 - protein, 9 aa, Homo sapiens
KLQCVDLHV

SEQ ID NO: 83 - protein, 9 aa, Homo sapiens
ELAHYDVLL

SEQ ID NO: 84 - protein, 9 aa, Homo sapiens
NLNGAGDPL

SEQ ID NO: 85 - protein, 9 aa, Homo sapiens
TLRVDCTPL

SEQ ID NO: 86 - protein, 9 aa, Homo sapiens
MMNDQLMFL

SEQ ID NO: 87 - protein, 9 aa, Homo sapiens
ALFDIESKV

SEQ ID NO: 88 - protein, 9 aa, Homo sapiens
LLHETDSAV

SEQ ID NO: 89 - protein, 9 aa, Homo sapiens
VLAKELKFV

SEQ ID NO: 90 - protein, 9 aa, Homo sapiens
ILLWQPIPV

SEQ ID NO: 91 - protein, 9 aa, Homo sapiens
DLFGIWSKV

SEQ ID NO: 92 - protein, 9 aa, Homo sapiens
PLERFAELV

SEQ ID NO: 93 - protein, 9 aa, Homo sapiens
KQGNFNAWV

SEQ ID NO: 94 - protein, 9 aa, Homo sapiens
NLLRRMWVT

SEQ ID NO: 95 - protein, 9 aa, Homo sapiens
NLFETPILA

SEQ ID NO: 96 - protein, 9 aa, Homo sapiens
NLFETPVEA

SEQ ID NO: 97 - protein, 9 aa, Homo sapiens
GLQHWVPEL

SEQ ID NO: 98 - protein, 9 aa, Homo sapiens
VQFVASYKV

SEQ ID NO: 99 - protein, 9 aa, Homo sapiens
RLLAALCGA

SEQ ID NO: 100 - protein, 9 aa, Homo sapiens
LLLLTVLTV

SEQ ID NO: 101 - protein, 9 aa, Homo sapiens
LLLTVLTVV

SEQ ID NO: 102 - protein, 9 aa, Homo sapiens
FLSFHISNL

SEQ ID NO: 103 - protein, 9 aa, Homo sapiens
LLVLVCVLV

SEQ ID NO: 104 - protein, 9 aa, Homo sapiens
ALLVLVCVL

SEQ ID NO: 105 - protein, 9 aa, Homo sapiens
SLSYTNPAV

SEQ ID NO: 106 - protein, 9 aa, Homo sapiens
NLTISDVSV

SEQ ID NO: 107 - protein, 9 aa, Homo sapiens
ALASTAPPV

SEQ ID NO: 108 - protein, 9 aa, Homo sapiens
AILCWTFWV

SEQ ID NO: 109 - protein, 9 aa, Homo sapiens
FILMFIVYA

SEQ ID NO: 110 - protein, 9 aa, Homo sapiens
LTAECIFFV

SEQ ID NO: 111 - protein, 9 aa, Homo sapiens
MLQDNCCGV

SEQ ID NO: 112 - protein, 9 aa, Homo sapiens
ILCWTFWVL

SEQ ID NO: 113 - protein, 9 aa, Homo sapiens
KILLAYFIL

SEQ ID NO: 114 - protein, 9 aa, Homo sapiens
FVGICLFCL

SEQ ID NO: 115 - protein, 9 aa, Homo sapiens
VLLSVAMFL

SEQ ID NO: 116 - protein, 9 aa, Homo sapiens
LLSVAMFLL

SEQ ID NO: 117 - protein, 9 aa, Homo sapiens
ILGSLPFFL

SEQ ID NO: 118 - protein, 9 aa, Homo sapiens
ILNAYLVRV

SEQ ID NO: 119 - protein, 9 aa, Homo sapiens
FLLVGFAGA

SEQ ID NO: 120 - protein, 9 aa, Homo sapiens
NLQPQLASV

SEQ ID NO: 121 - protein, 9 aa, Homo sapiens
CMFDSKEAL

SEQ ID NO: 122 - protein, 9 aa, Homo sapiens
YLYVLVDSA

SEQ ID NO: 123 - protein, 9 aa, Homo sapiens
YMDGTMSQV

SEQ ID NO: 124 - protein, 9 aa, Homo sapiens
KMARFSYSV

SEQ ID NO: 125 - protein, 9 aa, Homo sapiens
GLVMDEHLV

SEQ ID NO: 126 - protein, 9 aa, Homo sapiens
FLPGCDGLV

SEQ ID NO: 127 - protein, 9 aa, Homo sapiens
CMLGSFCAC

SEQ ID NO: 128 - protein, 9 aa, Homo sapiens
YLAFRDDSI

SEQ ID NO: 129 - protein, 9 aa, Homo sapiens
WLPKKCSLC

SEQ ID NO: 130 - protein, 9 aa, Homo sapiens
CLNGGTCML

SEQ ID NO: 131 - protein, 9 aa, Homo sapiens
MLVGICLSI

SEQ ID NO: 132 - protein, 9 aa, Homo sapiens
FELGLVAGL

SEQ ID NO: 133 - protein, 9 aa, Homo sapiens
KMVRFSYSV

SEQ ID NO: 134 - protein, 9 aa, Homo sapiens
CLNEGTCML

SEQ ID NO: 135 - protein, 9 aa, Homo sapiens
MLAGICLSI

SEQ ID NO: 136 - protein, 9 aa, Homo sapiens
RLLFFLLFL

SEQ ID NO: 137 - protein, 9 aa, Homo sapiens
TLAYLIFCL

SEQ ID NO: 138 - protein, 9 aa, Homo sapiens
LLFLTPMEV

SEQ ID NO: 139 - protein, 9 aa, Homo sapiens
KLMSPKLYV

SEQ ID NO: 140 - protein, 9 aa, Homo sapiens
LLFFLLFLV

SEQ ID NO: 141 - protein, 9 aa, Homo sapiens
SLFLGILSV

SEQ ID NO: 142 - protein, 9 aa, Homo sapiens
AISGMILSI

SEQ ID NO: 143 - protein, 9 aa, Homo sapiens
FIRAHTPYI

SEQ ID NO: 144 - protein, 9 aa, Homo sapiens
SLNFIRAHT

SEQ ID NO: 145 - protein, 9 aa, Homo sapiens
LKMESLNFI

SEQ ID NO: 146 - protein, 9 aa, Homo sapiens
SHFLKMESL

SEQ ID NO: 147 - protein, 9 aa, Homo sapiens
YLFLGILSV

SEQ ID NO: 148 - protein, 9 aa, Homo sapiens
HLVEALYLV

SEQ ID NO: 149 - protein, 9 aa, Homo sapiens
LNIDLLWSV

SEQ ID NO: 150 - protein, 9 aa, Homo sapiens
VLFGLGFAI

SEQ ID NO: 151 - protein, 9 aa, Homo sapiens
VYLKTNVFL

SEQ ID NO: 152 - protein, 9 aa, Homo sapiens
VYLKTNLFL

SEQ ID NO: 153 - protein, 9 aa, Homo sapiens
KYNKANAFL

SEQ ID NO: 154 - protein, 9 aa, Homo sapiens
KYNIANVFL

SEQ ID NO: 155 - protein, 9 aa, Homo sapiens
KYNKANVFL

SEQ ID NO: 156 - protein, 9 aa, Homo sapiens
FQDENYLYL

SEQ ID NO: 157 - protein, 9 aa, Homo sapiens
LYLVCGERG

SEQ ID NO: 158 - protein, 15 aa, Homo sapiens
SLQPLALEGSLQSRG

SEQ ID NO: 159 - protein, 13 aa, Homo sapiens
QHLQKDYRAYYTF

SEQ ID NO: 160 - protein, 13 aa, Homo sapiens
NFFRMVISNPAAT

SEQ ID NO: 161 - protein, 13 aa, Homo sapiens
NFIRMVISNPAAT

SEQ ID NO: 162 - protein, 13 aa, Homo sapiens
YTFLNFMSNVGDP

SEQ ID NO: 163 - protein, 13 aa, Homo sapiens
FFYTPKTRREAED

SEQ ID NO: 164 - protein, 15 aa, Homo sapiens
SLQPLALEGSLQKRG

SEQ ID NO: 165 - protein, 9 aa, Homo sapiens
LYLVCGERI

SEQ ID NO: 166 - protein, 9 aa, Homo sapiens
KYQAVTTTL

SEQ ID NO: 167 - protein, 9 aa, Homo sapiens
KYCLITIFL

SEQ ID NO: 168 - protein, 9 aa, Homo sapiens
ALWMRLLPL

SEQ ID NO: 169 - protein, 9 aa, Homo sapiens
LWMRLLPLL

SEQ ID NO: 170 - protein, 9 aa, Homo sapiens
RLLPLLALL

SEQ ID NO: 171 - protein, 10 aa, Homo sapiens
HLCGSHLVEA

SEQ ID NO: 172 - protein, 9 aa, Homo sapiens
ALYLVCGER

SEQ ID NO: 173 - protein, 10 aa, Homo sapiens
LYLVCGERGF

SEQ ID NO: 174 - protein, 9 aa, Homo sapiens
LVCGERGFF

SEQ ID NO: 175 - protein, 10 aa, Homo sapiens
VCGERGFFYT

SEQ ID NO: 176 - protein, 8 aa, Homo sapiens
GERGFFYT

SEQ ID NO: 177 - protein, 9 aa, Homo sapiens
ERGFFYTPK

SEQ ID NO: 178 - protein, 9 aa, Homo sapiens
FYTPKTRRE

SEQ ID NO: 179 - protein, 11 aa, Homo sapiens
TPKTRREAEDL

SEQ ID NO: 180 - protein, 9 aa, Homo sapiens
SLQPLALEG

SEQ ID NO: 181 - protein, 9 aa, Homo sapiens
ALEGSLQKR

SEQ ID NO: 182 - protein, 10 aa, Homo sapiens
SLQKRGIVEQ

SEQ ID NO: 183 - protein, 10 aa, Homo sapiens
GIVEQCCTSI

SEQ ID NO: 184 - protein, 9 aa, Homo sapiens
IVEQCCTSI

SEQ ID NO: 185 - protein, 9 aa, Homo sapiens
SLYQLENYC

SEQ ID NO: 186 - protein, 21 aa, Homo sapiens
MEVGWYRSPFSRVVHLYRNGK

SEQ ID NO: 187 - protein, 20 aa, Homo sapiens
EVGWYRSPFSRVVHLYRNGK

SEQ ID NO: 188 - protein, 9 aa, Homo sapiens
SLLLELEEV

SEQ ID NO: 189 - protein, 9 aa, Homo sapiens
LMWAKIGPV

SEQ ID NO: 190 - protein, 9 aa, Homo sapiens
VLFSSDFRI

SEQ ID NO: 191 - protein, 9 aa, Homo sapiens
SLSRFSWGA

SEQ ID NO: 192 - protein, 9 aa, Homo sapiens
KVEDPFYWV

SEQ ID NO: 193 - protein, 10 aa, Homo sapiens
RTFDPHFLRV

SEQ ID NO: 194 - protein, 9 aa, Homo sapiens
FLRVPCWKI

SEQ ID NO: 195 - protein, 10 aa, Homo sapiens
KITLFVIVPV

SEQ ID NO: 196 - protein, 9 aa, Homo sapiens
VLGPLVALI

SEQ ID NO: 197 - protein, 9 aa, Homo sapiens
TLFVIVPVL

SEQ ID NO: 198 - protein, 10 aa, Homo sapiens
RLAGQFLEEL

SEQ ID NO: 199 - protein, 9 aa, Homo sapiens
FLYGALLLA

SEQ ID NO: 200 - protein, 13 aa, Homo sapiens
TCFFRDHSYQEEA

SEQ ID NO: 201 - protein, 13 aa, Homo sapiens
TCFFRDHSYQSEA

SEQ ID NO: 202 - protein, 13 aa, Homo sapiens
VHFFKNIVTPRTP

SEQ ID NO: 203 - protein, 18 aa, Homo sapiens
YIYFNTWTTCQSIAFPSK

SEQ ID NO: 204 - protein, 15 aa, Homo sapiens
GAVRQIFGDYKTTIC

SEQ ID NO: 205 - protein, 13 aa, Homo sapiens
PVVHFFKNIVTPR

SEQ ID NO: 206 - protein, 15 aa, Homo sapiens
NYQDYEYLINVIHAF

SEQ ID NO: 207 - protein, 15 aa, Homo sapiens
ATLVSLLTFMIAATY

SEQ ID NO: 208 - protein, 15 aa, Homo sapiens
LVLLAVLPVLLLQIT

SEQ ID NO: 209 - protein, 15 aa, Homo sapiens
FLRVPCWKITLFVIV

SEQ ID NO: 210 - protein, 15 aa, Homo sapiens
RHPIRALVGDEVELP

SEQ ID NO: 211 - protein, 15 aa, Homo sapiens
RVPCWKITLFVIVPV

SEQ ID NO: 212 - protein, 15 aa, Homo sapiens
TLVSLLTFMIAATYN

SEQ ID NO: 213 - protein, 20 aa, Homo sapiens
KYLATASTMDHARHGFLPRH

SEQ ID NO: 214 - protein, 17 aa, Homo sapiens
ENPVVHFFKNIVTPRTP

SEQ ID NO: 215 - protein, 19 aa, Homo sapiens
LSRFSWGAEGQRPGFGYGG

SEQ ID NO: 216 - protein, 25 aa, Homo sapiens
AQGTLSKIFKLGGRDSRSGSPMARR

SEQ ID NO: 217 - protein, 15 aa, Homo sapiens
ALIICYNWLHRRLAG

SEQ ID NO: 218 - protein, 15 aa, Homo sapiens
IGPRHPIRALVGDEV

SEQ ID NO: 219 - protein, 15 aa, Homo sapiens
AEGFYTTGAVRQIFG

SEQ ID NO: 220 - protein, 16 aa, Homo sapiens
HCLGKWLGHPDKFVGI

SEQ ID NO: 221 - protein, 12 aa, Homo sapiens
QLQPFPQPELPY

SEQ ID NO: 222 - protein, 11 aa, Homo sapiens
PQPELPYPQPE

SEQ ID NO: 223 - protein, 13 aa, Homo sapiens
SGEGSFQPSQQNP

SEQ ID NO: 224 - protein, 14 aa, Homo sapiens
GDLIAEVETDKATV

SEQ ID NO: 225 - protein, 14 aa, Homo sapiens
GDLLAEIETDKATI

SEQ ID NO: 226 - protein, 15 aa, Homo sapiens
GDLLAEIETDKATIG

SEQ ID NO: 227 - protein, 15 aa, Homo sapiens
AQWLAEFRKYLEKPI

SEQ ID NO: 228 - protein, 15 aa, Homo sapiens
RLLLQLLGSPGRRYY

SEQ ID NO: 229 - protein, 15 aa, Homo sapiens
GRVFVSPLAKKLAVE

SEQ ID NO: 230 - protein, 15 aa, Homo sapiens
DIPISNIRRVIAQRL

SEQ ID NO: 231 - protein, 15 aa, Homo sapiens
SPGRRYYSLPPHQKV

SEQ ID NO: 232 - protein, 15 aa, Homo sapiens
ETIANDVVSLATKAR

SEQ ID NO: 233 - protein, 14 aa, Homo sapiens
GEIRTMNNFLDREI

SEQ ID NO: 234 - protein, 15 aa, Homo sapiens
FGIFVVDKNTGDINI

SEQ ID NO: 235 - protein, 15 aa, Homo sapiens
CECNIKVKDVNDNFP

SEQ ID NO: 236 - protein, 15 aa, Homo sapiens
NKAEFHQSVISRYRV

SEQ ID NO: 237 - protein, 15 aa, Homo sapiens
DSTFIVNKTITAEVL

SEQ ID NO: 238 - protein, 15 aa, Homo sapiens
SITTLNATSALLRAQ

SEQ ID NO: 239 - protein, 15 aa, Homo sapiens
ILSSELLRFQVTDLD

SEQ ID NO: 240 - protein, 15 aa, Homo sapiens
EGILKVVKALDYEQL

SEQ ID NO: 241 - protein, 15 aa, Homo sapiens
STPVTIQVINVREGI

SEQ ID NO: 242 - protein, 15 aa, Homo sapiens
AIFVVVILVHGELRI

SEQ ID NO: 243 - protein, 15 aa, Homo sapiens
RTNEGILKVVKALDY

SEQ ID NO: 244 - protein, 15 aa, Homo sapiens
DSKTAEIKFVKNMNR

SEQ ID NO: 245 - protein, 15 aa, Homo sapiens
KREWIKFAAACREGE

SEQ ID NO: 246 - protein, 15 aa, Homo sapiens
MFIINRNTGEIRTMN

SEQ ID NO: 247 - protein, 15 aa, Homo sapiens
SQYKLKASAISVTVL

SEQ ID NO: 248 - protein, 15 aa, Homo sapiens
WSFFRVVAMLFIFLV

SEQ ID NO: 249 - protein, 15 aa, Homo sapiens
SKIAFKIIRQEPSDS

SEQ ID NO: 250 - protein, 15 aa, Homo sapiens
TNVGILKVVKPLDYE

SEQ ID NO: 251 - protein, 15 aa, Homo sapiens
MDWSFFRVVAMLFIF

SEQ ID NO: 252 - protein, 15 aa, Homo sapiens
KNGTIKWHSIRRQKR

SEQ ID NO: 253 - protein, 15 aa, Homo sapiens
RTNVGILKVVKPLDY

SEQ ID NO: 254 - protein, 15 aa, Homo sapiens
GAGILYLVTPPSVVG

SEQ ID NO: 255 - protein, 15 aa, Homo sapiens
RSQVETDDLILKPGV

SEQ ID NO: 256 - protein, 14 aa, Homo sapiens
EKPLPVDMVLISLC

SEQ ID NO: 257 - protein, 15 aa, Homo sapiens
TAEFLAMLIFVLLSL

SEQ ID NO: 258 - protein, 15 aa, Homo sapiens
LRQMRTVTPIRMQGG

SEQ ID NO: 259 - protein, 15 aa, Homo sapiens
AVNIVGYSNAQGVDY

SEQ ID NO: 260 - protein, 15 aa, Homo sapiens
RFGISNYCQIYPPNV

SEQ ID NO: 261 - protein, 15 aa, Homo sapiens
PCIIHRGKPFQLEAV

SEQ ID NO: 262 - protein, 15 aa, Homo sapiens
TVKVMGDDGVLACAI

SEQ ID NO: 263 - protein, 15 aa, Homo sapiens
EAINQGYMHADAYPF

SEQ ID NO: 264 - protein, 15 aa, Homo sapiens
KDLTYTFLRDFEQYL

SEQ ID NO: 265 - protein, 15 aa, Homo sapiens
RQLRTLVNEAINQGY

SEQ ID NO: 266 - protein, 15 aa, Homo sapiens
MDKIRYRLVYNRQNT

SEQ ID NO: 267 - protein, 15 aa, Homo sapiens
LNQRKIYLKTNVYLK

SEQ ID NO: 268 - protein, 15 aa, Homo sapiens
EYILYLQGIELGYWK

SEQ ID NO: 269 - protein, 15 aa, Homo sapiens
TCATLLIHQGVAITT

SEQ ID NO: 270 - protein, 15 aa, Homo sapiens
AKHMRQLRTLVNEAI

SEQ ID NO: 271 - protein, 15 aa, Homo sapiens
IRYRLVYNRQNTLNR

SEQ ID NO: 272 - protein, 15 aa, Homo sapiens
ENFIRINGKRWLYFK

SEQ ID NO: 273 - protein, 15 aa, Homo sapiens
TGAAATYAIDSIADA

SEQ ID NO: 274 - protein, 15 aa, Homo sapiens
NATFSMDQLKFGDTI

SEQ ID NO: 275 - protein, 15 aa, Homo sapiens
DRTVVSSIGAYKLIQ

SEQ ID NO: 276 - protein, 15 aa, Homo sapiens
MVVQHNLRAMNSNRM

SEQ ID NO: 277 - protein, 15 aa, Homo sapiens
KMRKQIRGLSQASLN

SEQ ID NO: 278 - protein, 15 aa, Homo sapiens
GAYKLIQKELGLASS

SEQ ID NO: 279 - protein, 15 aa, Homo sapiens
QHNLRAMNSNRMLGI

SEQ ID NO: 280 - protein, 15 aa, Homo sapiens
YKLIQKELGLASSIG

SEQ ID NO: 281 - protein, 15 aa, Homo sapiens
ADDIVKMLNDPALNR

SEQ ID NO: 282 - protein, 15 aa, Homo sapiens
HNIQVADDARFVLNA

SEQ ID NO: 283 - protein, 15 aa, Homo sapiens
ADDARFVLNAGKKKF

SEQ ID NO: 284 - protein, 15 aa, Homo sapiens
GCISYALVSHTAKGS

SEQ ID NO: 285 - protein, 15 aa, Homo sapiens
LPVTVTLDIITAPLQ

SEQ ID NO: 286 - protein, 15 aa, Homo sapiens
SGCISYALVSHTAKG

SEQ ID NO: 287 - protein, 15 aa, Homo sapiens
DIVKMLNDPALNRHN

SEQ ID NO: 288 - protein, 15 aa, Homo sapiens
ARFVLNAGKKKFTGT

SEQ ID NO: 289 - protein, 24 aa, Homo sapiens
TYTEHAKRKTVTAMDVVYALKRQG

SEQ ID NO: 290 - protein, 15 aa, Homo sapiens
EHAKRKTVTAMDVVY

SEQ ID NO: 291 - protein, 15 aa, Homo sapiens
AKRKTVTAMDVVYAL

SEQ ID NO: 292 - protein, 15 aa, Homo sapiens
HAKRKTVTAMDVVYA

SEQ ID NO: 293 - protein, 15 aa, Homo sapiens
RKTVTAMDVVYALKR

SEQ ID NO: 294 - protein, 14 aa, Homo sapiens
TVTAMDVVYALKRQ

SEQ ID NO: 295 - protein, 15 aa, Homo sapiens
PKKGSKKAVTKAQKK

SEQ ID NO: 296 - protein, 15 aa, Homo sapiens
KAVTKAQKKDGKKRK

SEQ ID NO: 297 - protein, 21 aa, Homo sapiens
STDHPKYSDMIVAAIQAEKNR

SEQ ID NO: 298 - protein, 15 aa, Homo sapiens
KYSDMIVAAIQAEKN

SEQ ID NO: 299 - protein, 15 aa, Homo sapiens
SQEYSGSVANEANVY

SEQ ID NO: 300 - protein, 15 aa, Homo sapiens
SHSLPYESSISTALE

SEQ ID NO: 301 - protein, 15 aa, Homo sapiens
TGAYSNASSTESASY

SEQ ID NO: 302 - protein, 15 aa, Homo sapiens
SFLSQEYSGSVANEA

SEQ ID NO: 303 - protein, 20 aa, Homo sapiens
KTTKQSFDLSVKAQYKKNKH

SEQ ID NO: 304 - protein, 15 aa, Homo sapiens
KTTKQSFDLSVKAQY

SEQ ID NO: 305 - protein, 15 aa, Homo sapiens
TTKQSFDLSVKAQYK

SEQ ID NO: 306 - protein, 15 aa, Homo sapiens
GALVPGGVADAAAAY

SEQ ID NO: 307 - protein, 15 aa, Homo sapiens
AAQFGLVGAAGLGGL

SEQ ID NO: 308 - protein, 15 aa, Homo sapiens
APRPGVLLLLLSILH

SEQ ID NO: 309 - protein, 15 aa, Homo sapiens
GGVADAAAAYKAAKA

SEQ ID NO: 310 - protein, 15 aa, Homo sapiens
VLLLLLSILHPSRPG

SEQ ID NO: 311 - protein, 15 aa, Homo sapiens
AAKAAQFGLVGAAGL

SEQ ID NO: 312 - protein, 15 aa, Homo sapiens
VAAKAQLRAAAGLGA

SEQ ID NO: 313 - protein, 15 aa, Homo sapiens
KSAAKVAAKAQLRAA

SEQ ID NO: 314 - protein, 15 aa, Homo sapiens
KAQLRAAAGLGAGIP

SEQ ID NO: 315 - protein, 15 aa, Homo sapiens
VPGALAAAKAAKYGA

SEQ ID NO: 316 - protein, 15 aa, Homo sapiens
RPTMDGDPDTPKPVS

SEQ ID NO: 317 - protein, 15 aa, Homo sapiens
SYKEAVLRAIDGINQ

SEQ ID NO: 318 - protein, 15 aa, Homo sapiens
NQRSSDANLYRLLDL

SEQ ID NO: 319 - protein, 15 aa, Homo sapiens
KRIVQRIKDFLRNLV

SEQ ID NO: 320 - protein, 15 aa, Homo sapiens
EFKRIVQRIKDFLRN

SEQ ID NO: 321 - protein, 15 aa, Homo sapiens
RIVQRIKDFLRNLVP

SEQ ID NO: 322 - protein, 15 aa, Homo sapiens
RFALLGDFFRKSKEK

SEQ ID NO: 323 - protein, 15 aa, Homo sapiens
QRIKDFLRNLVPRTE

SEQ ID NO: 324 - protein, 15 aa, Homo sapiens
DGRYVLNGHWVVSPP

SEQ ID NO: 325 - protein, 15 aa, Homo sapiens
THVVYTRDTGPQETL

SEQ ID NO: 326 - protein, 15 aa, Homo sapiens
RLLHYCGSDFVFQAR

SEQ ID NO: 327 - protein, 15 aa, Homo sapiens
HDLLLQVLLQEPNPG

SEQ ID NO: 328 - protein, 15 aa, Homo sapiens
ETRYEVRIQLVYKNR

SEQ ID NO: 329 - protein, 15 aa, Homo sapiens
HRDYLMAVQRLVSPD

SEQ ID NO: 330 - protein, 15 aa, Homo sapiens
EGHAFYHSFGRVLDG

SEQ ID NO: 331 - protein, 15 aa, Homo sapiens
RNHLALMGGDGRYVL

SEQ ID NO: 332 - protein, 15 aa, Homo sapiens
NPGIEFEFWLPRERY

SEQ ID NO: 333 - protein, 15 aa, Homo sapiens
VQRVFRDAGAFAGYW

SEQ ID NO: 334 - protein, 15 aa, Homo sapiens
QLVYKNRSPLRAREY

SEQ ID NO: 335 - protein, 15 aa, Homo sapiens
RRFEYDDPRFLRLLD

SEQ ID NO: 336 - protein, 15 aa, Homo sapiens
TPVVVLNGLAAVREA

SEQ ID NO: 337 - protein, 15 aa, Homo sapiens
ENLRIVVADLFSAGM

SEQ ID NO: 338 - protein, 20 aa, Homo sapiens
TLAWGLLLMILHPDVQRRVQ

SEQ ID NO: 339 - protein, 20 aa, Homo sapiens
TTLITNLSSVLKDEAVWEKP

SEQ ID NO: 340 - protein, 15 aa, Homo sapiens
DPRFLRLLDLAQEGL

SEQ ID NO: 341 - protein, 15 aa, Homo sapiens
RMELFLFFTSLLQHF

SEQ ID NO: 342 - protein, 15 aa, Homo sapiens
DLFSAGMVTTSTTLA

SEQ ID NO: 343 - protein, 15 aa, Homo sapiens
ELFLFFTSLLQHFSF

SEQ ID NO: 344 - protein, 15 aa, Homo sapiens
DQLRRRFGDVFSLQL

SEQ ID NO: 345 - protein, 15 aa, Homo sapiens
EQRRFSVSTLRNLGL

SEQ ID NO: 346 - protein, 20 aa, Homo sapiens
RRFEYDDPRFLRLLDLAQEG

SEQ ID NO: 347 - protein, 20 aa, Homo sapiens
AGMVTTSTTLAWGLLLMILH

SEQ ID NO: 348 - protein, 15 aa, Homo sapiens
QRRFSVSTLRNLGLG

SEQ ID NO: 349 - protein, 15 aa, Homo sapiens
ESGFLREVLNAVPVL

SEQ ID NO: 350 - protein, 15 aa, Homo sapiens
GKVLRFQKAFLTQLD

SEQ ID NO: 351 - protein, 15 aa, Homo sapiens
DIEVQGFRIPKGTTL

SEQ ID NO: 352 - protein, 15 aa, Homo sapiens
KPEAFLPFSAGRRAC

SEQ ID NO: 353 - protein, 15 aa, Homo sapiens
YKKLLKERKEMFSYL

SEQ ID NO: 354 - protein, 15 aa, Homo sapiens
DELRTDLKAVEAKVQ

SEQ ID NO: 355 - protein, 15 aa, Homo sapiens
NKITNSLVLDIIKLA

SEQ ID NO: 356 - protein, 14 aa, Homo sapiens
NRLDRCLKAVRKER

SEQ ID NO: 357 - protein, 12 aa, Homo sapiens
LIQQGARVGRID

SEQ ID NO: 358 - protein, 15 aa, Homo sapiens
SPSLDVLITLLSLGS

SEQ ID NO: 359 - protein, 15 aa, Homo sapiens
DQKSCFKSMITAGFE

SEQ ID NO: 360 - protein, 15 aa, Homo sapiens
YTFRGFMSHTNNYPC

SEQ ID NO: 361 - protein, 15 aa, Homo sapiens
YNERLLHTPHNPISL

SEQ ID NO: 362 - protein, 15 aa, Homo sapiens
DCILCIHSTTSCFAP

SEQ ID NO: 363 - protein, 15 aa, Homo sapiens
SSLLNKITNSLVLDI

SEQ ID NO: 364 - protein, 15 aa, Homo sapiens
GSSLLNKITNSLVLD

SEQ ID NO: 365 - protein, 15 aa, Homo sapiens
NDSFIQEISKMYPGR

SEQ ID NO: 366 - protein, 15 aa, Homo sapiens
KEMFSYLSNQIKKLS

SEQ ID NO: 367 - protein, 15 aa, Homo sapiens
STLELFLHELAIMDS

SEQ ID NO: 368 - protein, 15 aa, Homo sapiens
NSLVLDIIKLAGVHT

SEQ ID NO: 369 - protein, 15 aa, Homo sapiens
SKCMHLIQQGARVGR

SEQ ID NO: 370 - protein, 15 aa, Homo sapiens
RSHEHLIRLLLEKGK

SEQ ID NO: 371 - protein, 15 aa, Homo sapiens
RRHYRFIHGIGRSGD

SEQ ID NO: 372 - protein, 15 aa, Homo sapiens
SNGYKKLLKERKEMF

SEQ ID NO: 373 - protein, 16 aa, Homo sapiens
QFFMSDKPLHLAVSLN

SEQ ID NO: 374 - protein, 15 aa, Homo sapiens
DVIGLTFRRDLYFSR

SEQ ID NO: 375 - protein, 15 aa, Homo sapiens
NVVLYSSDYYVKPVA

SEQ ID NO: 376 - protein, 15 aa, Homo sapiens
SSVRLLIRKVQHAPL

SEQ ID NO: 377 - protein, 15 aa, Homo sapiens
EVPFRLMHPQPEDPA

SEQ ID NO: 378 - protein, 15 aa, Homo sapiens
KKIKAFVEQVANVVL

SEQ ID NO: 379 - protein, 15 aa, Homo sapiens
STPTKLQESLLKKLG

SEQ ID NO: 380 - protein, 15 aa, Homo sapiens
KKVYVTLTCAFRYGQ

SEQ ID NO: 381 - protein, 15 aa, Homo sapiens
KTLTLLPLLANNRER

SEQ ID NO: 382 - protein, 16 aa, Homo sapiens
NRERRGIALDGKIKHE

SEQ ID NO: 383 - protein, 15 aa, Homo sapiens
EAAWQFFMSDKPLHL

SEQ ID NO: 384 - protein, 15 aa, Homo sapiens
QFFMSDKPLHLAVSL

SEQ ID NO: 385 - protein, 15 aa, Homo sapiens
TFIQFKKDLKESMKC

SEQ ID NO: 386 - protein, 15 aa, Homo sapiens
DTGALDVIRNFTLDM

SEQ ID NO: 387 - protein, 15 aa, Homo sapiens
EVIHLIEEHRLVREH

SEQ ID NO: 388 - protein, 15 aa, Homo sapiens
REYRKKMDIPAKLIV

SEQ ID NO: 389 - protein, 15 aa, Homo sapiens
EEILKALDAAFYKTF

SEQ ID NO: 390 - protein, 15 aa, Homo sapiens
KRFLLAVDVSASMNQ

SEQ ID NO: 391 - protein, 15 aa, Homo sapiens
TDMTLQQVLMAMSQI

SEQ ID NO: 392 - protein, 15 aa, Homo sapiens
EVWKALLQEMPLTAL

SEQ ID NO: 393 - protein, 15 aa, Homo sapiens
SHKDLLRLSHLKPSS

SEQ ID NO: 394 - protein, 15 aa, Homo sapiens
YKTFKTVEPTGKRFL

SEQ ID NO: 395 - protein, 15 aa, Homo sapiens
ETEKLLKYLEAVEKV

SEQ ID NO: 396 - protein, 15 aa, Homo sapiens
RIHPFHILIALETYK

SEQ ID NO: 397 - protein, 15 aa, Homo sapiens
EKDSYVVAFSDEMVP

SEQ ID NO: 398 - protein, 15 aa, Homo sapiens
TPADVFIVFTDNETF

SEQ ID NO: 399 - protein, 15 aa, Homo sapiens
QKLGLENAEALIRLI

SEQ ID NO: 400 - protein, 15 aa, Homo sapiens
KLLKKARIHPFHILI

SEQ ID NO: 401 - protein, 15 aa, Homo sapiens
DDQTCREDLHILFSN

SEQ ID NO: 402 - protein, 15 aa, Homo sapiens
TDEYKNDVKNRSVYI

SEQ ID NO: 403 - protein, 15 aa, Homo sapiens
SIFVVFDSIESAKKF

SEQ ID NO: 404 - protein, 15 aa, Homo sapiens
TDLLILFKDDYFAKK

SEQ ID NO: 405 - protein, 15 aa, Homo sapiens
HQIEYYFGDFNLPRD

SEQ ID NO: 406 - protein, 15 aa, Homo sapiens
KEQIKLDEGWVPLEI

SEQ ID NO: 407 - protein, 15 aa, Homo sapiens
DKGQVLNIQMRRTLH

SEQ ID NO: 408 - protein, 15 aa, Homo sapiens
EIMIKFNRLNRLTTD

SEQ ID NO: 409 - protein, 15 aa, Homo sapiens
RDKFLKEQIKLDEGW

SEQ ID NO: 410 - protein, 15 aa, Homo sapiens
SEDKTKIRRSPSKPL

SEQ ID NO: 411 - protein, 15 aa, Homo sapiens
QVLNIQMRRTLHKAF

SEQ ID NO: 412 - protein, 15 aa, Homo sapiens
RNKEVTWEVLEGEVE

SEQ ID NO: 413 - protein, 15 aa, Homo sapiens
NRLTTDFNVIVEALS

SEQ ID NO: 414 - protein, 15 aa, Homo sapiens
KGSIFVVFDSIESAK

SEQ ID NO: 415 - protein, 15 aa, Homo sapiens
KTKIRRSPSKPLPEV

SEQ ID NO: 416 - protein, 15 aa, Homo sapiens
IFVVFDSIESAKKFV

SEQ ID NO: 417 - protein, 15 aa, Homo sapiens
KERIANFKIEPPGLF

SEQ ID NO: 418 - protein, 15 aa, Homo sapiens
QGSIKYIMLNPSSRI

SEQ ID NO: 419 - protein, 15 aa, Homo sapiens
QREKFAWAIDMADED

SEQ ID NO: 420 - protein, 15 aa, Homo sapiens
IQGSIKYIMLNPSSR

SEQ ID NO: 421 - protein, 15 aa, Homo sapiens
YNASITLQQQLKELT

SEQ ID NO: 422 - protein, 15 aa, Homo sapiens
EQLMKLEVQATDREE

SEQ ID NO: 423 - protein, 15 aa, Homo sapiens
SQYFKAQTEARKQMS

SEQ ID NO: 424 - protein, 15 aa, Homo sapiens
YIMLNPSSRIKGEKD

SEQ ID NO: 425 - protein, 15 aa, Homo sapiens
KILSYNRANRAVAIL

SEQ ID NO: 426 - protein, 15 aa, Homo sapiens
KLIEDEFIIDESDQS

SEQ ID NO: 427 - protein, 15 aa, Homo sapiens
KVYKTLDTPFFSTGK

SEQ ID NO: 428 - protein, 15 aa, Homo sapiens
QDILVFYVNFGDLLC

SEQ ID NO: 429 - protein, 15 aa, Homo sapiens
KVMLKKIEIDNKVSD

SEQ ID NO: 430 - protein, 15 aa, Homo sapiens
EDNIMTAQNVPLKPQ

SEQ ID NO: 431 - protein, 15 aa, Homo sapiens
TREIILMDLVRPQDT

SEQ ID NO: 432 - protein, 15 aa, Homo sapiens
TSVSQNVIPSSAQKR

SEQ ID NO: 433 - protein, 15 aa, Homo sapiens
RIRDSEYEIQRQAKK

SEQ ID NO: 434 - protein, 15 aa, Homo sapiens
TYQFFVKHGELKVYK

SEQ ID NO: 435 - protein, 15 aa, Homo sapiens
DEEFYLSVGSPSVLL

SEQ ID NO: 436 - protein, 15 aa, Homo sapiens
REIILMDLVRPQDTY

SEQ ID NO: 437 - protein, 15 aa, Homo sapiens
YQFFVKHGELKVYKT

SEQ ID NO: 438 - protein, 15 aa, Homo sapiens
HDGYSLDGPEEIECT

SEQ ID NO: 439 - protein, 15 aa, Homo sapiens
KCSYTEDAQCIDGTI

SEQ ID NO: 440 - protein, 15 aa, Homo sapiens
GYSLDGPEEIECTKL

SEQ ID NO: 441 - protein, 15 aa, Homo sapiens
KVSFFCKNKEKKCSY

SEQ ID NO: 442 - protein, 15 aa, Homo sapiens
DLPFSTVVPLKTFYE

SEQ ID NO: 443 - protein, 15 aa, Homo sapiens
ECLPQHAMFGNDTIT

SEQ ID NO: 444 - protein, 15 aa, Homo sapiens
CKVPVKKATVVYQGE

SEQ ID NO: 445 - protein, 15 aa, Homo sapiens
YSCKPGYVSRGGMRK

SEQ ID NO: 446 - protein, 15 aa, Homo sapiens
KKATVVYQGERVKIQ

SEQ ID NO: 447 - protein, 15 aa, Homo sapiens
EVPKCFKEHSSLAFW

SEQ ID NO: 448 - protein, 15 aa, Homo sapiens
VPKCFKEHSSLAFWK

SEQ ID NO: 449 - protein, 15 aa, Homo sapiens
KCFKEHSSLAFWKTD

SEQ ID NO: 450 - protein, 15 aa, Homo sapiens
QPFMFRLDNRYQPME

SEQ ID NO: 451 - protein, 15 aa, Homo sapiens
RIKNQADCIPFFRSC

SEQ ID NO: 452 - protein, 15 aa, Homo sapiens
SSLRCMVDLGPCWAG

SEQ ID NO: 453 - protein, 15 aa, Homo sapiens
QQRQALAQISLPRII

SEQ ID NO: 454 - protein, 15 aa, Homo sapiens
RSLMFMQWGQLLDHD

SEQ ID NO: 455 - protein, 15 aa, Homo sapiens
QEARKIVGAMVQIIT

SEQ ID NO: 456 - protein, 15 aa, Homo sapiens
DNRYQPMEPNPRVPL

SEQ ID NO: 457 - protein, 15 aa, Homo sapiens
ELLSYFKQPVAATRT

SEQ ID NO: 458 - protein, 15 aa, Homo sapiens
QLGTVLRNLKLARKL

SEQ ID NO: 459 - protein, 15 aa, Homo sapiens
YLPLVLGPTAMRKYL

SEQ ID NO: 460 - protein, 15 aa, Homo sapiens
LGTVLRNLKLARKLM

SEQ ID NO: 461 - protein, 15 aa, Homo sapiens
KNNIFMSNSYPRDFV

SEQ ID NO: 462 - protein, 15 aa, Homo sapiens
SLQMRGNPGSHFCGG

SEQ ID NO: 463 - protein, 15 aa, Homo sapiens
TRVALYVDWIRSTLR

SEQ ID NO: 464 - protein, 15 aa, Homo sapiens
TLIHPSFVLTAAHCL

SEQ ID NO: 465 - protein, 15 aa, Homo sapiens
NDVLLIQLSSPANLS

SEQ ID NO: 466 - protein, 15 aa, Homo sapiens
DPPAQVLQELNVTVV

SEQ ID NO: 467 - protein, 15 aa, Homo sapiens
HCLRDIPQRLVNVVL

SEQ ID NO: 468 - protein, 15 aa, Homo sapiens
DWIRSTLRRVEAKGR

SEQ ID NO: 469 - protein, 15 aa, Homo sapiens
RPHNICTFVPRRKAG

SEQ ID NO: 470 - protein, 15 aa, Homo sapiens
HPSFVLTAAHCLRDI

SEQ ID NO: 471 - protein, 15 aa, Homo sapiens
DVLLIQLSSPANLSA

SEQ ID NO: 472 - protein, 15 aa, Homo sapiens
YVDWIRSTLRRVEAK

SEQ ID NO: 473 - protein, 15 aa, Homo sapiens
EYVTLKKMREIIGWP

SEQ ID NO: 474 - protein, 15 aa, Homo sapiens
NFFRMVISNPAATHQ

SEQ ID NO: 475 - protein, 15 aa, Homo sapiens
DSVILIKCDERGKMI

SEQ ID NO: 476 - protein, 11 aa, Homo sapiens
AHHPIWARMDA

SEQ ID NO: 477 - protein, 18 aa, Homo sapiens
TAALSYTISRMEESSVTL

SEQ ID NO: 478 - protein, 19 aa, Homo sapiens
LHRSGVLIIHHLQEDYRTY

SEQ ID NO: 479 - protein, 9 aa, Homo sapiens
RHGLDNYRG

SEQ ID NO: 480 - protein, 12 aa, Homo sapiens
GWYRSPFSRVVH

SEQ ID NO: 481 - protein, 12 aa, Homo sapiens
TCFFRDHSYQEE

SEQ ID NO: 482 - protein, 15 aa, Homo sapiens
GIAGFKGDQGPKGET

SEQ ID NO: 483 - protein, 11 aa, Homo sapiens
LSIALHVGFDH

SEQ ID NO: 484 - DNA, 21 bp, synthetic construct
  misc_feature 1..21: note = source = /note="Description of Artificial Sequence: Syntheticprimer"
  source 1..21: mol_type = other DNA
tcatcattgacctcgtggccc

SEQ ID NO: 485 - DNA, 24 bp, synthetic construct
  misc_feature 1..24: note = source = /note="Description of Artificial Sequence: Syntheticprimer"
  source 1..24: mol_type = other DNA
atcgtacttctccacttgcaatcc

SEQ ID NO: 486 - DNA, 20 bp, synthetic construct
  misc_feature 1..20: note = source = /note="Description of Artificial Sequence: Syntheticprimer"
  source 1..20: mol_type = other DNA
cttgcactaccaaagccaca

SEQ ID NO: 487 - DNA, 20 bp, synthetic construct
  misc_feature 1..20: note = source = /note="Description of Artificial Sequence: Syntheticprimer"
  source 1..20: mol_type = other DNA
gttattgtcttcccggctgt

SEQ ID NO: 488 - DNA, 20 bp, synthetic construct
  misc_feature 1..20: note = source = /note="Description of Artificial Sequence: Syntheticprimer"
  source 1..20: mol_type = other DNA
agcagttggtgaccatgtcg

SEQ ID NO: 489 - DNA, 20 bp, synthetic construct
  misc_feature 1..20: note = source = /note="Description of Artificial Sequence: Syntheticprimer"
  source 1..20: mol_type = other DNA
tggagatctcctgcttgagg

SEQ ID NO: 490 - DNA, 24 bp, synthetic construct
  misc_feature 1..24: note = source = /note="Description of Artificial Sequence: Syntheticprimer"
  source 1..24: mol_type = other DNA
tgaacgctacacactgcatcttgg

SEQ ID NO: 491 - DNA, 23 bp, synthetic construct
  misc_feature 1..23: note = source = /note="Description of Artificial Sequence: Syntheticprimer"
  source 1..23: mol_type = other DNA
cgactccttttccgcttcctgag

SEQ ID NO: 492 - DNA, 20 bp, synthetic construct
  misc_feature 1..20: note = source = /note="Description of Artificial Sequence: Syntheticprimer"
  source 1..20: mol_type = other DNA
tcccaaatccttcgggttac

SEQ ID NO: 493 - DNA, 20 bp, synthetic construct
  misc_feature 1..20: note = source = /note="Description of Artificial Sequence: Syntheticprimer"
  source 1..20: mol_type = other DNA
gagctagactctgcggcgta

SEQ ID NO: 494 - DNA, 20 bp, synthetic construct
  misc_feature 1..20: note = source = /note="Description of Artificial Sequence: Syntheticprimer"
  source 1..20: mol_type = other DNA
ccgggtgctacaaaagtcat

SEQ ID NO: 495 - DNA, 20 bp, synthetic construct
  misc_feature 1..20: note = source = /note="Description of Artificial Sequence: Syntheticprimer"
  source 1..20: mol_type = other DNA
gtcggccacattgaaaaagt

SEQ ID NO: 496 - DNA, 21 bp, synthetic construct
  misc_feature 1..21: note = source = /note="Description of Artificial Sequence: Syntheticprimer"
  source 1..21: mol_type = other DNA
cgtccctgcatcccactactt

SEQ ID NO: 497 - DNA, 19 bp, synthetic construct
  misc_feature 1..19: note = source = /note="Description of Artificial Sequence: Syntheticprimer"
  source 1..19: mol_type = other DNA
ggacatggccccagcatag

SEQ ID NO: 498 - DNA, 20 bp, synthetic construct
  misc_feature 1..20: note = source = /note="Description of Artificial Sequence: Syntheticprimer"
  source 1..20: mol_type = other DNA
tgacccacctccttttcaag

SEQ ID NO: 499 - DNA, 20 bp, synthetic construct
  misc_feature 1..20: note = source = /note="Description of Artificial Sequence: Syntheticprimer"
  source 1..20: mol_type = other DNA
ttagggtcatgcacactgga

SEQ ID NO: 500 - DNA, 20 bp, synthetic construct
  misc_feature 1..20: note = source = /note="Description of Artificial Sequence: Syntheticprimer"
  source 1..20: mol_type = other DNA
ctggaacggtgaaggtgaca

SEQ ID NO: 501 - DNA, 23 bp, synthetic construct
  misc_feature 1..23: note = source = /note="Description of Artificial Sequence: Syntheticprimer"
  source 1..23: mol_type = other DNA
aagggacttcctgtaacaatgca

SEQ ID NO: 502 - DNA, 20 bp, synthetic construct
  misc_feature 1..20: note = source = /note="Description of Artificial Sequence: Syntheticprimer"
  source 1..20: mol_type = other DNA
aagacccagacatcaaggcg

SEQ ID NO: 503 - DNA, 20 bp, synthetic construct
  misc_feature 1..20: note = source = /note="Description of Artificial Sequence: Syntheticprimer"
  source 1..20: mol_type = other DNA
aatcgatgacagcgccgtag

SEQ ID NO: 504 - protein, 6 aa, synthetic construct
  REGION 1..6: note = source = /note="Description of Artificial Sequence: Synthetic6xHis tag"
HHHHHH